(19) United States
(12) Patent Application Publication
(10) Pub. No.: US 2004/0010119 A1
(43) Pub. Date: Jan. 15, 2004

(54) Novel proteins and nucleic acids encoding same

(76) Inventors: Xiaojia Guo, Branford, CT (US); Elma Fernandes, Branford, CT (US); Li Li, Branford, CT (US); Ramesh Kekuda, Stamford, CT (US); Yi Liu, New Haven, CT (US); Mario Leite, Milford, CT (US); Kimberly A. Spytek, New Haven, CT (US); Weizhen Ji, Branford, CT (US); Stacie J. Casman, North Haven, CT (US); Ference L. Boldog, North Haven, CT (US); Meera Patturajan, Branford, CT (US); Corine A. M. Vernet, Branford, CT (US); Robert A. Ballinger, Newington, CT (US); Uriel M. Malyankar, Branford, CT (US); Velizar T. Tchernev, Branford, CT (US); Angela D. Blalock, Branford, CT (US); Vladimir Y. Gusev, Madison, CT (US); Luca Rastelli, Guilford, CT (US); Peter D. Mezes, Old Lyme, CT (US); Karen Ellerman, Branford, CT (US); Melvyn Heyes, New Haven, CT (US); John L. Herrmann, Guilford, CT (US); Richard A. Shimkets, Guilford, CT (US); Noelle Ioime, Hamden, CT (US); Carol E. A. Pena, New Haven, CT (US); Suresh G. Shenoy, Branford, CT (US); Raymond J. Taupier JR., East Haven, CT (US); Valerie Gerlach, Branford, CT (US); Linda Gorman, East Haven, CT (US)

Correspondence Address: Ivor R. Elrifi, Mintz, Levin, Cohn, Ferris, Glovsky and Popeo, P.C., One Financial Center, Boston, MA, 02111, US

(21) Appl. No.: 10/074,978

(22) Filed: Feb. 12, 2002

Related U.S. Application Data
(60) Provisional application No. 60/268,221, filed on Feb. 12, 2001.
(60) Provisional application No. 60/268,496, filed on Feb. 13, 2001.
(60) Provisional application No. 60/268,665, filed on Feb. 14, 2001.
(60) Provisional application No. 60/268,646, filed on Feb. 14, 2001.
(60) Provisional application No. 60/269,136, filed on Feb. 15, 2001.
(60) Provisional application No. 60/269,310, filed on Feb. 16, 2001.
(60) Provisional application No. 60/269,530, filed on Feb. 16, 2001.
(60) Provisional application No. 60/276,405, filed on Mar. 15, 2001.
(60) Provisional application No. 60/276,703, filed on Mar. 16, 2001.
(60) Provisional application No. 60/276,399, filed on Mar. 16, 2001.
(60) Provisional application No. 60/278,199, filed on Mar. 23, 2001.
(60) Provisional application No. 60/279,274, filed on Mar. 28, 2001.
(60) Provisional application No. 60/280,238, filed on Mar. 30, 2001.
(60) Provisional application No. 60/280,899, filed on Apr. 2, 2001.
(60) Provisional application No. 60/310,797, filed on Aug. 8, 2001.
(60) Provisional application No. 60/312,284, filed on Aug. 14, 2001.
(60) Provisional application No. 60/322,294, filed on Sep. 14, 2001.
(60) Provisional application No. 60/322,295, filed on Sep. 14, 2001.
(60) Provisional application No. 60/330,293, filed on Oct. 18, 2001.
(60) Provisional application No. 60/335,104, filed on Oct. 31, 2001.
(60) Provisional application No. 60/335,109, filed on Oct. 31, 2001.
(60) Provisional application No. 60/332,127, filed on Nov. 21, 2001.
(60) Provisional application No. 60/331,772, filed on Nov. 21, 2001.

(51) Int. Cl. C12Q 1/68; C07H 21/04; A61K 38/17; C07K 14/435; C07K 14/47; C12P 21/02; C12N 5/06

(52) U.S. Cl. 530/350; 514/12; 435/6; 435/69.1; 435/320.1; 435/325; 536/23.2

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This is a request for filing a new nonprovisional application under 37 C.F.R. §1.53(b). This application claims priority to U.S. S. No. 60/268,221 filed on Feb. 12, 2001 (Cura 569); U.S. S. No. 60/335,109 filed on Oct. 31, 2001 (Cura 569 F1); U.S. S. No. 60/312,284 filed on Aug. 14, 2001 (Cura 569 IFC-01); U.S. S. No. 60/268,496 filed on Feb. 13, 2001 (Cura 270); U.S. S. No. 60/276,703 filed on Mar. 16, 2001 (Cura 570 B1); U.S. S. No. 60/330,293 filed on Oct. 18, 2001 (Cura 570 C1); U.S. S. No. 60/322,127 filed on Nov. 21, 2001 (Cura 570 F IFC-02); U.S. S. No. 60/280,899 filed on Apr. 2, 2001 (Cura 570 G1); U.S. S. No. 60/310,797 filed on Aug. 8, 2001 (Cura 570 IFC-01); U.S. S. No. 60/268,646 field on Feb. 14, 2001 (Cura 571); U.S. S. No. 60/276,399 filed on Mar. 16, 2001 (Cura 571 A); U.S. S. No. 60/268,665 filed on Feb. 14, 2001 (Cura 572); U.S. S. No. 60/269,530 filed on Feb. 16, 2001 (Cura 572 A); U.S. S. No. 60/269,136 filed on Feb. 15, 2001 (Cura 573); U.S. S. No. 60/279,274 filed on Mar. 28, 2001 (Cura 573 Al); U.S. S. No. 60/331,772 filed on Nov. 21, 2001 (Cura 573 D IFC-01); U.S. S. No. 60/322,295 filed on Sep. 14, 2001 (Cura 573 I1); U.S. S. No. 60/278,199 filed on Mar. 23, 2001 (Cura 573 J1); U.S. S. No. 60/276,405 filed on Mar. 15, 2001 (Cura 573 J11); U.S. S. No. 60/269,310 filed on Feb. 16, 2001 (Cura 574); U.S. S. No. 60/280,238 filed on Mar. 30, 2001 (Cura 574 A1); U.S. S. No. 60/322,294 filed on Sep. 14, 2001 (Cura 574 B1); U.S. S. No. 60/335,104 filed on Oct. 31, 2001 (Cura 574 D1); each of which is incorporated by reference in its entirety.

### FIELD OF THE INVENTION

The invention relates to polynucleotides and the polypeptides encoded by such polynucleotides, as well as vectors, host cells, antibodies and recombinant methods for producing the polypeptides and polynucleotides, as well as methods for using the same.

### BACKGROUND OF THE INVENTION

The present invention is based in part on nucleic acids encoding proteins that are new members of the following protein families: Zinc Finger-like proteins, Pepsin A Precursor-like proteins, Ribonuclease Pancreatic-like proteins, Ser/Thr Protein Kinase-like proteins, Glycodelin-like proteins, Neuropathy Target Esterase/Swiss Cheese Protein-like proteins, Acid-Sensitive Potassium Channel Protein Task-like protein, Novel Ribosomal Protein L8-like proteins, Prostaglandin Omega Hydroxylase-like proteins, Myeloid Upregulated Protein-like proteins, Testicular Serine Protease-like proteins, Hepatitis B Virus (HBV) Associated Factor-like proteins, Apolipoprotein L-like proteins, Rh Type C Glycoprotein-like proteins, Copine III-like protiens, Carboxypeptidase B Pancreatic-like proteins, Ribosomal Protein L29-like proteins, Ser/Thr kinase-like proteins, Metallaproteinase-Disintegrin (ADAM30)-like proteins, Bone Morphogenetic Protein 11-like proteins, Protein Tyrosine Phosphatase-like proteins, Aldo-Keto Reductase Family 7, Member A3-like proteins, Ral Guanine Nucleotide Exchange Factor 3-like proteins, Endolyn-like proteins, Arylacetamide Deacetylase-like proteins, GPCR-like proteins, PB39-like proteins, Oxytocin-like proteins, Thymosin beta-4-like proteins, beta Thymosin-like proteins, Thymosin Beta-4-like proteins, Mylein P2-like proteins, Testis Lipid-Binding Protein-like proteins, Intracellular Thrombospondin Domain Containing Protein-like protein, Ornithine Decarboxylase-like protein, Short-Chain Dehydrogenase/Reductase-like protein, Protocadherin Beta 3-like protein and Adrenomedullin Receptor-like protein. More particularly, the invention relates to nucleic acids encoding novel polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, NOV10, NOV11, NOV12, NOV13, NOV14, NOV15, NOV16, NOV17, NOV18, NOV19, NOV20, NOV21, NOV22, NOV23, NOV24, NOV25, NOV26, NOV27, NOV28, NOV29, NOV30, NOV31, NOV32, NOV33, NOV34, NOV35, NOV36, and NOV37 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, and 111. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112. The nucleic acid can be, for example, a genomic DNA fragment or a cDNA molecule that includes the nucleic acid sequence of any of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, and 111.

Also included in the invention is an oligonucleotide, e.g., an oligonucleotide which includes at least 6 contiguous nucleotides of a NOVX nucleic acid (e.g., SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, and 111) or a complement of said oligonucleotide. Also included in the invention are substantially purified NOVX polypeptides (SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., trauma, regeneration (in vitro and in vivo); Von Hippel-Lindau (VHL) syndrome; Alzheimer's disease; stroke; Tuberous sclerosis; hypercalceimia; Parkinson's disease, Huntington's disease; Cerebral palsy; Epilepsy; Lesch-Nyhan syndrome; multiple sclerosis; Ataxia-telangiectasia; leukodystrophies; behavioral disorders; addiction, anxiety, pain; actinic keratosis; acne; hair growth diseases; allopecia; pigmentation disorders; endocrine disorders; connective tissue disorders (such as severe neonatal Marfan syndrome dominant ectopia lentis, familial ascending aortic aneurysm and isolated skeletal features of Marfan syndrome); Shprintzen-Goldberg syndrome; genodermatoses; contractural arachnodactyly; inflammatory disorders such as osteo- and rheumatoid-arthritis; inflammatory bowel disease; Crohn's disease; immunological disorders; AIDS; cancers including but not limited to lung cancer, colon cancer, neoplasm, adenocarcinoma, lymphoma, prostate cancer, uterus cancer, leukemia or pancreatic cancer; blood disorders; asthma; psoriasis; vascular disorders, hypertension, skin disorders, renal disorders including Alport syndrome; immunological disorders; tissue injury; fibrosis disorders; bone diseases; Ehlers-Danlos syndrome type VI, VII, type IV, S-linked cutis laxa and Ehlers-Danlos syndrome type V; osteogenesis imperfecta; neurologic diseases; brain disorders like encephalomyelitis; neurodegenerative disorders; immune disorders; hematopoietic disorders; muscle disorders; inflammation and wound repair; parasitic, bacterial, fungal, protozoal and viral infections (particularly infections caused by HIV-1 or HIV-2), acute heart failure; hypotension; hypertension; urinary retention; osteoporosis; treatment of Albright hereditary ostoeodystrophy; angina pectoris; myocardial infarction; ulcers; benign prostatic hypertrophy; arthrogryposis multiplex congenita; osteogenesis imperfecta; keratoconus; scoliosis; duodenal atresia; esophageal atresia; intestinal malrotation; pancreatitis; obesity; systemic lupus erythematosus; autoimmune disease; emphysema; scleroderma; allergy; ARDS; neuroprotection; fertility; Myasthenia gravis; diabetes; growth and reproductive disorders; hemophilia; hypercoagulation; idiopathic thrombocytopenic purpura; immunodeficiencies; graft versus host; adrenoleukodystrophy; congenital adrenal hyperplasia; endometriosis; xerostomia; ulcers; cirrhosis; transplantation; diverticular disease; Hirschsprung's disease; appendicitis; arthritis; ankylosing spondylitis; tendinitis; renal artery stenosis; interstitial nephritis; glomerulonephritis; polycystic kidney disease; erythematosus; renal tubular acidosis; IgA nephropathy; anorexia; bulimia; psychotic disorders; including schizophrenia, manic depression, delirium, and dementia; severe mental retardation and dyskinesias, and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g. a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

NOVX nucleic acids and polypeptides are further useful in the generation of antibodies that bind immuno-specifically to the novel NOVX substances for use in therapeutic or diagnostic methods. These NOVX antibodies may be generated according to methods known in the art, using prediction from hydrophobicity charts, as described in the “Anti-NOVX Antibodies” section below. The disclosed NOVX proteins have multiple hydrophilic regions, each of which can be used as an immunogen. These NOVX proteins can be used in assay systems for functional analysis of various human disorders, which will help in understanding of pathology of the disease and development of new drug targets for various disorders.

The NOVX nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 depicts an electrophoresis profile for angiopoietin related protein (ARP), panel A and vascular endothelial growth factor (VEGF), panel B; and a TaqMan expression profile for VEGF (panel C) and for ARP (panel D).

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE 1
Sequences and Corresponding SEQ ID Numbers

| | | | Nucleic | Amino |
|---|---|---|---|---|
| | | | Acid | Acid |
| NOVX | | | SEQ ID | SEQ ID |
| No. | Internal Acc. No. | Homology | NO. | NO. |
| 1 | CG56920-01 | Zinc Finger Protein-like Proteins | 1 | 2 |
| 2 | CG57107-01 | Pepsin A Precursor-like Protein | 3, 5, 7, | 4, 6, 8, |
| | | | 9, 11 | 10, 12 |
| 3 | CG56936-01 | Ribonuclease Pancreatic-like | 13 | 14 |
| | | Proteins | | |
| 4 | CG51707-02 | Ser/Thr Protein Kinase-like | 15 | 16 |
| | | Proteins | | |
| 5 | CG57081-01 | Ser/Thr Protein Kinase-like | 17 | 18 |
| | | Proteins | | |
| 6 | CG56684-02 | Glycodelin-like Proteins | 19 | 20 |
| 7 | CG56977-01 | Neuropathy Target Esterase/Swiss | 21 | 22 |
| | | Cheese Protein-like Proteins | | |
| 8 | CG57119-01 | Acid-Sensitive potassium Channel | 23 | 24 |
| | | Protein Task-like Proteins | | |
| 9 | CG57143-01 | Novel Ribosomal Protein L8-like | 25 | 26 |
| | | Proteins | | |
| 10 | CG56860-01 | Prostaglandin Omega Hydroxylase- | 27 | 28 |
| | | like Proteins | | |
| 11 | CG57024-01 | Myeloid Upregulated Protein-like | 29 | 30 |
| | | Proteins | | |
| 12 | CG57083-01 | Testicular Serine Protease-like | 31 | 32 |
| | | Proteins | | |
| 13a | CG56961-01 | Hepatitis B Virus (HBV) | 33 | 34 |
| | | Associated Factor-like Proteins | | |
| 13b | CG56961-02 | Hepatitis B Virus (HBV) | 35 | 36 |
| | | Associated Factor-like Proteins | | |
| 14 | CG57104-01 | Apolipoprotein L-like Proteins | 37 | 38 |
| 14b | CG57104-02 | Apolipoprotein L-like Proteins | 39 | 40 |
| 15 | CG57146-01 | Rh Type C Glycoprotein-like | 41 | 42 |
| | | Protein | | |
| 16 | CG57169-01 | Copine III-like Protein | 43 | 44 |
| 17 | CG57177-01 | Carboxypeptidase B, Pancreatic- | 45, 47, | 46, 48, |
| | | like Proteins | 49, 51, 53 | 50, 52, |
| | | | | 54 |
| 18a | CG57113-01 | Ribosomal Protein L29-like | 55 | 56 |
| | | Proteins | | |
| 18b | CG57113-02 | Ribosomal Protein L29-like | 57 | 58 |
| | | Proteins | | |
| 19 | CG57211-01 | Metalloproteinase-Disintegrin | 59 | 60 |
| | | (ADAM30)-like Proteins | | |
| 20 | CG57222-01 | Bone Morphongenetic Protein 11- | 61 | 62 |
| | | like Proteins | | |
| 21a | CG56477-01 | Adrenomedullin Receptor-like | 63 | 64 |
| | | Protein | | |
| 21b | CG56477-02 | Adrenomedullin Receptor-like | 65 | 66 |
| | | Protein | | |
| 21c | CG56477-03 | Adrenomedullin Receptor-like | 67 | 68 |
| | | Protein | | |
| 22a | CG57256-01 | Protein Tyrosine Phosphatase-like | 69 | 70 |
| | | Proteins | | |
| 22b | CG57256-02 | Protein Tyrosine Phosphatase-like | 71 | 72 |
| | | Proteins | | |
| 23 | CG57228-01 | Aldo-Keto Reductase Family 7, | 73 | 74 |
| | | Member A3 like | | |
| 24 | CG57274-01 | Ral Guanine NucleotideExchange | 75 | 76 |
| | | Factor 3-like Proteins | | |
| 25 | CG57276-01 | Endolyn-like Proteins | 77 | 78 |
| 26 | CG57224-01 | Arylacetamide Deacetylase-like | 79 | 80 |
| | | Proteins | | |
| 27 | CG57288-01 | GPCR-like Proteins | 81 | 82 |
| 28 | CG57213-01 | PB39-like Proteins | 83 | 84 |
| 29 | CG56990-02 | Oxytocin-like Proteins | 85 | 86 |
| 30a | CG57330-01 | Thymosin beta-4-like Proteins | 87 | 88 |
| 30b | CG57330-03 | Beta Thymosin-like Proteins | 89 | 90 |
| 30c | CG57330-02 | Thymosin Beta-4-like Proteins | 91 | 92 |
| 31 | CG57344-01 | Myelin P2-like Proteins | 93 | 94 |
| 32a | CG57346-01 | Testis Lipid-binding Protein-like | 95 | 96 |
| | | Proteins | | |
| 32b | CG57346-02 | Testis Lipid-binding Protein-like | 97 | 98 |
| | | Proteins | | |
| 33 | CG57356-01 | Intracellular Thrombospondin | 99 | 100 |
| | | Domain Containing Protein-like | | |
| | | Protein | | |
| 34a | CG57258-01 | Ornithine Decarboxylase-like | 101 | 102 |
| | | Protein | | |
| 34b | CG57258-02 | Ornithine Decarboxylase-like | 103 | 104 |
| | | Protein | | |
| 34c | CG57258-03 | Ornithine Decarboxylase-like | 105 | 106 |
| | | Protein | | |
| 35 | CG57339-01 | Short-chain | 107 | 108 |
| | | Dehydrogenase/Reductase-like | | |
| | | Protein | | |
| 36 | CG57341-01 | Short-chain | 109 | 110 |
| | | Dehydrogenase/Reductase-like | | |
| | | Protein | | |
| 37 | CG57335-01 | Protocadherin Beta 3-like Protein | 111 | 112 |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1 is homologous to the Fibromodulin family of proteins. Thus, the NOV1 nucleic acids, polypeptides, antibodies and related compounds according to the invention will be useful in therapeutic and diagnostic applications implicated in, for example, the treatment of patients suffering from: repair of damage to cartilage and ligaments; therapeutic applications to joint repair, and other diseases, disorders and conditions of the like.

It has been suggested that fibromodulin participates in the assembly of the extracellular matrix by virtue of its ability to interact with type I and type II collagen fibrils and to inhibit fibrillogenesis in vitro. Additional utilities for the NOVX nucleic acids and polypeptides according to the invention are disclosed herein.

NOV1

A disclosed NOV1a (designated CuraGen Acc. No. CG56290-01) encodes a novel Zinc Finger Protein-like protein and includes the 1319 nucleotide sequence (SEQ ID NO:1) is shown in Table 1A. An open reading frame for the mature protein was identified beginning with an ATG initiation codon at nucleotides 445-447 and ending with a TAA stop codon at nucleotides 1228-1230. Putative untranslated regions are underlined in Table 1A, and the start and stop codons are in bold letters.

TABLE 1A
NOV1 Nucleotide Sequence

(SEQ ID NO: 1)

ACAGCCACAGTGATTTCATCCTTCGATACAGGGGATATACTGTACAGTCCTTTTTCTAGAAGTGAGACATACAAGA
TTACTCTACAAGAGGAAGATTCCAGGGGCTCAAAAACGCAAAGGTTTGCACTTTGAGAGCCCCTTGGAATGTTGAC
AACTCAGGATCTAAAACAAAGTTCTGTGTTAATGAGTTACAGAATTCACGTGGAAGTCAATGTCACTTTATAATCG
ATAATAATACTGAGTGAGGAACACTATGCAGGAAGAAACCTTCCGTAGAAAGACAGGCAGGGAAAAGCTTAGGCTG
ACCTTAAACTTACCTAATAGAGCAAGCCTGAGATAGACTGCCAAAATGGCCAAATAAGAGACTCTATGAAATAACA
GTCTTGTAACTGTAGTAATCATAAGGAAATTTTCTCCTTGAAATCACGATACCAAATAGGAAAAATGATCTACAAG
TGCCCCATGTGTAGGGAATTTTTCTCTGAGAGAGCAGATCTTTTTATGCATCAGAAAATTCACACAGCTGAGAAGC
CCCATAAATGTGACAAGTGTGATAAGGGTTTCTTTCATATATCAGAACTTCATATTCATTGGAGAGACCATACAGG
AGAGAAGGTCTATAAATGTGATGATTGTGGTAAGGATTTTAGCACTACAACAAAACTTAATAGACATAAGAAAATC
CACACAGTGGAGAAGCCCTATAAATGTTACGAGTGTGGCAAAGCCTTCAATTGGAGCTCCCATCTTCAAATTCATA
TGAGAGTTCATACAGGTGAGAAACCGTATGTCTGTAGTGAGTGTGGAAGGGGCTTTAGTAATAGTTCAAACCTTTG
CATGCATCAGAGAGTCCACACCGGAGAGAAGCCCTTTAAATGTGAAGAGTGTGGGAAGGCCTTCAGGCACACCTCC
AGCCTCTGCATGCATCAAAGAGTCCACACAGGAGAGAAACCCTATAAATGTTATGAGTGTGGGAAGGCGTTCAGTC
AGAGTTCGAGCCTCTGCATCCACCAGAGAGTCCACACTGGAGAGAAACCCTATAGATGTTGTGGATGTGGGAAGGC
CTTCAGTCAGAGTTCGGGCCTGTGCATCCACCAGAGAGTCCACACAGGAGAGAAACCTTTCAAATGTGATGAGTGC
GGAAAGGCCTTCAGTCAGAGTACGAGCCTCTGCATCCACCAGAGAGTCCACACAAAGGAGAGAAACCATCTCAAAA
TATCAGTTATATAAAACGTTTTGCTAAGAGTTTAAAATCTTAAAACCCATAAGTGCCACTAGGAAGGAAACCCTGT
ATCGAAGAATGAAATCACTGTGGCTGT

For all BLAST data described herein, public nucleotide databases include all GenBank databases and the GeneSeq patent database; and public amino acid databases include the GenBank databases, SwissProt, PDB and PIR.

The disclosed NOV1 nucleic acid sequence maps to chromosome 12q24.3 and invention has 901 of 1057 bases (85%) identical to a gb:GENBANK-ID:GPIZFPA|acc:L26335.1 mRNA from Cavia porcellus (Cavia porcellus zinc finger protein (zfoC1) mRNA, complete cds) (E=1.2e⁻¹⁶⁶).

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbict”) retrieved from the NOV1 BLAST analysis, e.g., Cavia porcellus zinc finger protein mRNA, matched the Query NOV1 sequence purely by chance is 10.2×10⁻¹⁶⁶. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNN”) or the letter “X” in protein sequences (e.g., “XXXXXXXXX”). Low-complexity regions can result in high scores that reflect compositional bias rather than significant position-by-position alignment. Wootton and Federhen, Methods Enzymol 266:554-571, 1996. Other BLAST results include sequences from the Patp database, which is a proprietary database that contains sequences published in patents and patent publications.

A disclosed NOV1 polypeptide (SEQ ID NO:2) is 261 amino acid residues in length and is presented using the one-letter amino acid code in Table 1B. The SignalP, Psort and/or Hydropathy results predict that NOV1 does not have a signal peptide and is likely to be localized to the mitochondrial matrix space with a certainty of 0.4401. In alternative embodiments, a NOV1 polypeptide is located to the microbody (peroxisome) with a certainty of 0.4294, the nucleus with a certainty of 0.3000, or in the mitochondrial inner membrane with a certainty of 0.1252.

TABLE 1B
Encoded NOV1 Protein Sequence

(SEQ ID NO: 2)

MIYKCPMCREFFSERADLFMHQKIHTAEKPHKCDKCDKGFFHISELHIHWRDHTGEKVYKCDDCGKDFSTTTKLN
RHKKIHTVEKPYKCYECGKAFNWSSHLQIHMRVHTGEKPYVCSECGRGFSNSSNLCMHQRVHTGEKPFKCEECGK
AFRHTSSLCMHQRVHTGEKPYKCYECGKAFSQSSSLCIHQRVHTGEKPYRCCGCGKAFSQSSGLCIHQRVHTGEK
PFKCDECGKAFSQSTSLCIHQRVHTKERNHLKISVI

The NOV1 amino acid sequence was found to have 258 of 261 amino acid residues (98%) identical to, and 259 of 261 amino acid residues (99%) similar to, the 261 amino acid residue ptnr:SPTREMBL-ACC:Q60493 protein from Cavia porcellus (Guinea pig) (ZINC FINGER PROTEIN) (E=1.9e⁻¹⁵²).

The Zinc Finger Protein-like gene disclosed in this invention is expressed in at least the following tissues: retina, and organ of Corti. Expression information was derived from the tissue sources of the sequences that were included in the derivation of the sequence of NOV1.

Possible small nucleotide polymorphisms (SNPS) found for NOV1 are listed in Tables 1C and 1D, where “PAF” is putative allelic frequency, the “>” sign means is changed to, “N/A” refers to a silent mutation, and “Depth” represents the number of clones covering the region of the SNP.

TABLE 1C
SNPs

| Consensus Position | Depth | Base Change | PAF |
|---|---|---|---|
| 1084 | 7 | G > A | N/A |

TABLE 1D
SNPs

| | Nucleotide | | Amino Acid | |
|---|---|---|---|---|
| Variant ID | Position | Base Change | Position | Base Change |
| 13376980 | 69 | A > G | NA | NA |
| 13376981 | 1081 | G > T | 213 | Gly > Ser |

Homologies to any of the above NOV1 proteins will be shared by other NOV1 proteins insofar as they are homologous to each other as shown above. Any reference to NOV1 is assumed to refer to both of the NOV1 proteins in general, unless otherwise noted.

NOV1 also has homology to the amino acid sequences shown in the BLASTP data listed in Table 1E.

TABLE 1E
BLAST results for NOV1

| Gene Index/ | | Length | Identity | Positives | |
|---|---|---|---|---|---|
| Identifier | Protein/Organism | (aa) | (%) | (%) | Expect |
| gi\|2144127\|pir\|\| | finger protein | 261 | 258/261 | 259/261 | e−123 |
| S70006 | zfOC1 - guinea pig | | (98%) | (98%) | |
| gi\|1196461\|gb\| | ZFOC1 gene product | 184 | 181/184 | 183/184 | 6e−84 |
| AAC41997.1\| | [Homo sapiens] | | (98%) | (99%) | |
| (L41669) | | | | | |
| gi\|2135119\| | finger protein | 183 | 180/183 | 182/183 | 2e−83 |
| pir\|\| | zfOC1 - human | | (98%) | (99%) | |
| S70007 | (fragment) | | | | |
| gi\|117445052\|ref\| | similar to zinc | 1147 | 151/253 | 187/253 | 1e−78 |
| XP_060551.1\| | finger protein 85 | | (59%) | (73%) | |
| (XM_060551) | (HPF4, HTF1) [Homo | | | | |
| | sapiens] | | | | |
| gi\|7019581\| | zinc finger | 606 | 155/246 | 184/246 | 1e−76 |
| ref\|NP_037381.1\| | protein 214 | | (63%) | (74%) | |
| (NM_013249) | [Homo | | | | |
| | sapiens] | | | | |

The homology of these sequences is shown graphically in the ClustalW analysis shown in Table 1F.

[image]

[image]

[image]

[image]

Tables 1G and 1H list the domain description from DOMAIN analysis results against NOV1. This indicates that the NOV1 sequence has properties similar to those of other proteins known to contain these domains. The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results may be collected from the Conserved Domain Database (CDD) with Reverse Position Specific BLAST analyses. This BLAST analysis software samples domains found in the Smart and Pfam collections. Sequences may also be analyzed according to a hmmpfam search against the HMM database (HMMER 2.1.1 (December 1998), Copyright (C) 1992-1998 Washington University School of Medicine). HMMER is freely distributed under the GNU General Public License.

For Table 1G and all successive DOMAIN sequence alignments, aligned residues are displayed in uppercase, residues identical (conserved) in the alignment between query (NOVX) and representative are shown in the extra line (I) between the two sequences, similar residues (“strong,” semionserved, with a positive score in the BLOSUM62 matrix) are indicated with a “+”. Regions masked out due to composition-bias are displayed in italics. The “strong” group of conserved amino acid residues may be any one of the following groups of amino acids: STA, NEQK, NHQK, NDEQ, QHRK, MILV, MILF, HY, FYW.

TABLE 1G
Domain Analysis of NOV1
HMM file: pfamHMMs
Scores for sequence family classification
(score includes all domains):

| Model | Description | Score | E-value | N |
|---|---|---|---|---|
| zf-C2H2 | Zinc finger, | 227.3 | 2.2e−64 | 9 |
| (InterPro) | C2H2 type | | | |

Parsed for domains:

| | | seq- | seq- | hmm- | hmm- | | |
|---|---|---|---|---|---|---|---|
| Model | Domain | from | to | from | to | score | E-value |
| zf-C2H2 | 1/9 | 3 | 25 . . . | 1 | 24 [ ] | 28.5 | 0.00016 |
| zf-C2H2 | 2/9 | 31 | 53 . . . | 1 | 24 [ ] | 21.4 | 0.021 |
| zf-C2H2 | 3/9 | 59 | 81 . . . | 1 | 24 [ ] | 32.4 | 1e−05 |
| zf-C2H2 | 4/9 | 87 | 109 . . . | 1 | 24 [ ] | 35.6 | 1.1e−06 |
| zf-C2H2 | 5/9 | 115 | 137 . . . | 1 | 24 [ ] | 35.4 | 1.3e−06 |
| zf-C2H2 | 6/9 | 143 | 165 . . . | 1 | 24 [ ] | 32.8 | 8e−06 |
| zf-C2H2 | 7/9 | 171 | 193 . . . | 1 | 24 [ ] | 34.1 | 3.3e−06 |
| zf-C2H2 | 8/9 | 199 | 221 . . . | 1 | 24 [ ] | 32.3 | 1.1e−05 |
| zf-C2H2 | 9/9 | 227 | 249 . . . | 1 | 24 [ ] | 34.1 | 3.2e−06 |

For example, Table 1H depicts the alignment of several regions of NOV1 with the zinc finger C2H2 consensus pattern YKCPFDCGKSFSRKSNLKRHLRTH (SEQ ID NO:118).

TABLE 1H
Alignments of top-scoring domains for NOV1

zf-C2H2: domain 1 of 9, from 3 to 25: score 28.5, E = 0.00016

| | | | | |
|---|---|---|---|---|
| | | *->ykCpfdCgksFsrksnLkrHlrtH<-* | | |
| | | \|\|\|\| \| + \|\| +++\| +\|+++\| | | |
| NOV1 | 3 | YKCP-MCREFFSERADLFMHQKIH | 25 | (SEQ ID NO: 119) |

zf-C2H2: domain 2 of 9, from 31 to 53: score 21.4, E = 0.021

| | | | | |
|---|---|---|---|---|
| | | *->ykCpfdCgksFsrksnLkrHlrtH<-* | | |
| | | +\|\|+ +\|+\| \| + \|+\|+ \|+\| \| | | |
| NOV1 | 31 | HKCD-KCDKGFFHISELHIHWRDH | 53 | (SEQ ID NO: 120) |

zf-C2H2: domain 3 of 9, from 59 to 81: score 32.4, E = 1e−05

| | | | | |
|---|---|---|---|---|
| | | *->ykCpfdCgksFsrksnLkrHlrtH<-* | | |
| | | \|\|\|+ \|\|\|\| \|\|+ \|+\|\|+++\| | | |
| NOV1 | 59 | YKCD-DCGKDFSTTTKLNRHKKIH | 81 | (SEQ ID NO: 121) |

zf-C2H2: domain 4 of 9, from 87 to 109: score 35.6, E = 1.1e−06

| | | | | |
|---|---|---|---|---|
| | | *->ykCpfdCgksFsrksnLkrHlrtH<-* | | |
| | | \|\|\| +\|\|\|+\|+ +\|+\|+ \|+\|+\| | | |
| NOV1 | 87 | YKCY-ECGKAFNWSSHLQIHMRVH | 109 | (SEQ ID NO: 122) |

zf-C2H2: domain 5 of 9, from 115 to 137: score 35.4, E = 1.3e−06

| | | | | |
|---|---|---|---|---|
| | | *->ykCpfdCgksFsrksnLkrHlrtH<-* | | |
| | | \|+\|+ +\|\|+ \|\|++\|\|\| +\|+\|+\| | | |
| NOV1 | 115 | YVCS-ECGRGFSNSSNLCMHQRVH | 137 | (SEQ ID NO: 123) |

zf-C2H2: domain 6 of 9, from 143 to 165: score 32.8, E = 8e−06

| | | | | |
|---|---|---|---|---|
| | | *->ykCpfdCgksFsrksnLkrHlrtH<-* | | |
| | | +\|\|+ +\|\|\|+\|++ \|+\| +\|+\|+\| | | |
| NOV1 | 143 | FKCE-ECGKAFRHTSSLCMHQRVH | 165 | (SEQ ID NO: 124) |

zf-C2H2: domain 7 of 9, from 171 to 193: score 34.1, E = 3.3e−06

| | | | | |
|---|---|---|---|---|
| | | *->ykCpfdCgksFsrksnLkrHlrtH<-* | | |
| | | \|\|\| +\|\|\|+\|\|++\|+\| \|+\|+\| | | |
| NOV1 | 171 | YKCY-ECGKAFSQSSSLCIHQRVH | 193 | (SEQ ID NO: 125) |

zf-C2H2: domain 8 of 9, from 199 to 221: score 32.3, E = 1.1e−05

| | | | | |
|---|---|---|---|---|
| | | *->ykCpfdCgksFsrksnLkrHlrtH+21 * | | |
| | | \|+\| +\|\|\|+\|\|++\| \| \|+\|+\| | | |
| NOV1 | 199 | YRCC-GCGKAFSQSSGLCIHQRVH | 221 | (SEQ ID NO: 126) |

zf-C2H2: domain 9 of 9, from 227 to 249: score 34.1, E = 3.2e−06

| | | | | |
|---|---|---|---|---|
| | | *->ykCpfdCgksFsrksnLkrHlrtH+21 -* | | |
| | | +\|\|+ +\|\|\|+\|\|++ +\| \|+\|+\| | | |
| NOV1 | 227 | FKCD-ECGKAFSQSTSLCIHQRVH | 249 | (SEQ ID NO: 127) |

Zinc finger domains are nucleic acid-binding protein structures first identified in the Xenopus transcription factor TFIIIA. These domains have since been found in numerous nucleic acid-binding proteins. A zinc finger domain is composed of 25 to 30 amino-acid residues. There are two cysteine or histidine residues at both extremities of the domain, which are involved in the tetrahedral coordination of a zinc atom. It has been proposed that such a domain interacts with about five nucleotides.

Many classes of zinc fingers are characterized according to the number and positions of the histidine and cysteine residues involved in the zinc atom coordination. In the first class to be characterized, called C2H2, the first pair of zinc coordinating residues are cysteines, while the second pair are histidines. A number of experimental reports have demonstrated the zinc-dependent DNA or RNA binding property of some members of this class.

A cDNA encoding a novel member of the zinc finger gene family, designated zfOC1, has been cloned from the organ of Corti. This cDNA is the first transcriptional regulator cloned from this sensory epithelium. This transcript encodes a peculiar protein composed of 9 zinc finger domains and a few additional amino acids. The deduced polypeptide shares 66% amino acid similarity with MOK-2, another protein of only zinc finger motifs and preferentially expressed in transformed cell lines. Northern blot hybridization analysis reveals that zfOC1 transcripts are predominantly expressed in the retina and the organ of Corti and at lower levels in the stria vascularis, auditory nerve, tongue, cerebellum, small intestine and kidney. Because of its relative abundance in sensorineural structures (retina and organ of Corti), this regulatory gene should be considered a candidate for hereditary disorders involving hearing and visual impairments that link to 12q24.3.

The protein similarity information, expression pattern, cellular localization, and map location for the NOV1 protein and nucleic acid disclosed herein suggest that this zinc finger protein-like protein may have important structural and/or physiological functions characteristic of the zinc finger protein family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: deafness, blindness as well as other diseases, disorders and conditions.

The novel nucleic acid encoding the Zinc Finger Protein-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV1 protein has multiple hydrophilic regions, each of which can be used as an immunogen. In one embodiment, a contemplated NOV1 epitope is from about amino acids 20 to 22 In another embodiment, a contemplated NOV1 epitope is from about amino acids 30 to 40. In other specific embodiments, contemplated NOV1 epitopes are from about amino acids 52 to 57, 70 to 80, 90 to 92, 105 to 120, 130 to 150, 160 to 180, 190 to 210, 220 to 240, and 245 to 248.

NOV2

A disclosed NOV2 nucleic acid (designated as CuraGen Acc. No. CG57107-01), which encodes a novel Pepsin A Precursor-like protein includes the 1688 nucleotide sequence (SEQ ID NO:3) shown in Table 2A. An open reading frame for the mature protein was identified beginning with and ATG codon at nucleotides 306-308 and ending with a TAA codon at nucleotides 1518-1520. Putative untranslated regions are underlined in Table 2A, and the start and stop codons are in bold letters.

TABLE 2A
NOV2 Nucleotide Sequence

(SEQ ID NO: 3)

TGCCTGTAGAGTTCAGCTGGTCAGGTQCGAGCACTGTCAAGCTAGCAGGGGCCTCCACTTGACCAGGGCATTGCGG
CCAAGGCAGCGGTAAGTGCCCTCATCACTGGGACGCACAGCCTGGATCTGCAGCCAGCCAGTCACCTCAAACCTCT
GGGGTCCACCCCTAAACTGCACAGAGATGTGGGGGTCATCCCCTGGCAGCTGGATGTCCAAGCCATCCTTCCTCCA
CTCGATGGAGGCCATGGGGTAGGCAAACACTTCACAGCCAAAGATCACATCCTGCCCTGTCACATTCCAAGTGTCA
TATGGATGTGACACGATCTTCTCCCTCGAGTTGGGACCCGGGAAGAAGCATGAAGTGGCTGCTGCTGCTGGGTCTG
GTGGCGCTCTCTGAGTGCATCATGTACAAGGTCCCCCTCATCAGAAAGAAGTCCTTGAGGCGCACCCTGTCCGAGC
GTGGCCTGCTGAAGGACTTCCTGAAGAAGCACAACCTCAACCCAGCCAGAAAGTACTTCCCCCAGTGGGAGGCTCC
CACCCTGGTAGATGAACAGCCCCTGGAGAACTACCTGGATATGGAGTACTTCGGCACTATCGGCATCGGAACTCCT
GCCCAGGATTTCACTGTCCTCTTTGACACCGGCTCCTCCAACCTGTGGGTGCCCTCAGTCTACTGCTCCAGTCTTG
CCTGCACCAACCACAACCGCTTCAACCCTGAGGATTCTTCCACCTACCAGGCCACCAGCGAGACAGTCTCCATCAC
CTACGGCACCGGCAGCATGACAGGCATCCTCGGATACGACACTGTCCAGGTTGGAGGCATCTCTGACACCAATCAG
ATCTTCGGCCTGAGCGAGACGGAACCTGGCTCCTTCCTGTATTATGCTCCCTTCGATGGCATCCTGGGOCTGGCCT
ACCCCAGCATTTCCTCCTCCGGGGCCACACCCGTCTTTCACAACATCTGGAACCAGGGCCTGGTTTCTCAGGACCT
CTTCTCTGTCTACCTCAGCGCCGATGACCAGAGTGGCAGCGTGGTGATCTTTGGTGGCATTGACTCTTCTTACTAC
ACTGGAAGTCTGAACTGGGTGCCTGTTACCGTCGAGGGTTACTGGCAGATCACCGTGGACAGCATCACCATGAACG
GAGAGGCCATCGCCTGCGCTGAGGGCTGCCAGGCCATTGTTGACACCGGCACCTCTCTGCTGACCGGCCCAACCAG
CCCCATTGCCAACATCCAGAGCGACATCGGAGCCAGCGAGAACTCAGATGGCGACATGGTGGTCAGCTGCTCAGCC
ATCAGCAGCCTGCCCGACATCGTCTTCACCATCAATGGAGTCCAGTACCCCGTGCCACCCAGTGCCTACATCCTGC
AGAGCGAGGGGAGCTGCATCAGTGGCTTCCAGGGCATGAACCTCCCCACCGAATCTGGAGAGCTTTGGATCCTGGG
TGATGTCTTCATCCGCCAGTACTTTACCGTCTTCGACAGGGCAAACAACCAGGTCAGCCTGGCCCCCGTGGCTTAA
GCCTAAGTCTCTTCAGCCACCTCCCAGGAAGATCTGGCCTCTGTCCTGTGCCCACTTTAGATGTATCTAATTCTCC
TGACTGTTCTTCCCAGGGGAGTGTGGAGGTCTTGGCCCTGTTCCCTGTCCTACCAATAACGTAGAATAAAAACATA
ACCCACCAAAAAAAAA

The nucleic acid sequence of NOV2 maps to chromosome 10q24 has 1285 of 1352 bases (95%) identical to a gb:GENBANK-ID:MFPEPA23|acc:X59755.1 mRNA from Macaca fuscata (M. fuscata mRNA for pepsinogen A-2/3) (E=5.6e⁻²⁷²).

A disclosed NOV2 polypeptide (SEQ ID NO:4) is 404 amino acid residues in length and is presented using the one-letter amino acid code in Table 2B. The SignalP, Psort and/or Hydropathy results predict that NOV2 is likely to be localized at the endoplasmic reticulum (membrane) with a certainty of 0.6000. In alternative embodiments, a NOV2 polypeptide is located to the microbody (peroxisome) with a certainty of 0.3788, the mitochondrial inner membrane with a certainty of 0.2567, or the plasma membrane with a certainty of 0.1000. The SignalP predicts a likely cleavage site for a NOV2 peptide between amino acid positions 31 and 32, i.e. at the sequence SEC-IM.

TABLE 2B
Encoded NOV2 Protein Sequence

(SEQ ID NO: 4)

MDVTRSSPSSWDPGRSMKWLLLLGLVALSECIMYKVPLIRKKSLRRTLSERGLLKDFLKKHNLNPARKYFPQ
WEAPTLVDEQPLENYLDMEYFGTIGIGTPAQDFTVLFDTGSSNLWVPSVYCSSLACTNHNRFNPEDSSTYQA
TSETVSITYGTGSMTGILGYDTVQVGGISDTNQIFGLSETEPGSFLYYAPFDGILGLAYPSISSSGATPVFD
NIWNQGLVSQDLFSVYLSADDQSGSVVIFGGIDSSYYTGSLNWVPVTVEGYWQITVDSITMNGEAIACAEGC
QAIVDTGTSLLTGPTSPIANIQSDIGASENSDGDMVVSCSAISSLPDIVFTINGVQYPVPPSAYILQSEGSC
ISGFQGMNLPTESGELWILGDVFIRQYFTVFDRANNQVSLAPVA

The NOV2 amino acid sequence was found to 385 of 388 amino acid residues (99%) identical to, and 387 of 388 amino acid residues (99%) similar to, the 388 amino acid residue ptnr:SWISSNEW-ACC:P00790 protein from Homo sapiens (Human) (PEPSIN A PRECURSOR (EC 3.4.23.1)) (E=1.0e⁻²⁰⁸).

NOV2 is expressed in at least the following tissues: stomach and testis. Expression information was derived from the tissue sources of the sequences that were included in the derivation of the sequence of NOV2.

Possible small nucleotide polymorphisms (SNPs) found for NOV2 are listed in Tables 2C.

TABLE 2C
SNPs

| | Nucleotide | | Amino Acid | |
|---|---|---|---|---|
| Variant | Position | Base Change | Position | Base Change |
| 13374720 | 386 | G > A | NA | NA |
| 13374721 | 525 | G > A | 74 | Glu > Lys |

Also included in the invention are four variants of NOV2: NOV2a (designated as CuraGen Acc. No. 175069704), NOV2b (designated as CuraGen Ace. No. 175069720), NOV2c (designated as CuraGen Ace. No. 175069724), and NOV2d (designated as CuraGen Acc. No. 175069728). An alignment of these sequences is given in Table 2D.

[image]

[image]

[image]

The proteins associated with NOV2a, NOV2b, NOV2c, and NOV2d are encoded in negative reading frames. An alignment of all NOV2 proteins is shown in Table 2E.

[image]

[image]

Homologies to any of the above NOV2 proteins will be shared by the other NOV2 proteins insofar as they are homologous to each other as shown above. Any reference to NOV2 is assumed to refer to the NOV2 proteins in general, unless otherwise noted.

NOV2 also has homology to the amino acid sequences shown in the BLASTP data listed in Table 2F.

TABLE 2F
BLAST results for NOV2

| Gene Index/ | | Length | Identity | Positives | |
|---|---|---|---|---|---|
| Identifier | Protein/Organism | (aa) | (%) | (%) | Expect |
| gi\|129792\|sp\| | Pepsin A precursor | 388 | 385/388 | 387/388 | 0.0 |
| P00790\|PEPA_{—} | | | (99%) | (99%) | |
| HUMAN | | | | | |
| gi\|625423\|pir\|\| | pepsin A (EC | 388 | 384/388 | 387/388 | 0.0 |
| A30142 | 3.4.23.1) 5 | | (98%) | (98%) | |
| | precursor - human | | | | |
| gi\|387013\|gb\| | pepsinogen A [Homo | 388 | 383/388 | 386/388 | 0.0 |
| AAA60061.1\| | sapiens] | | (98%) | (98%) | |
| (M26032) | | | | | |
| gi\|625424\|pir\|\| | pepsin A (EC | 388 | 382/388 | 386/388 | 0.0 |
| B30142 | 3.4.23.1) 4 | | (98%) | (99%) | |
| | precursor - human | | | | |
| gi\|129780\|sp\| | PEPSIN A-2/A-3 | 388 | 367/388 | 381/388 | 0.0 |
| P27677\|PEP2_MACFU | PRECURSOR (PEPSIN | | (94%) | (97%) | |
| | III-2/III-1) | | | | |

The homology of these sequences is shown graphically in the ClustalW analysis shown in Table 2G.

[image]

Table 2H lists the domain description from DOMAIN analysis results against NOV2. This indicates that the NOV2 sequence has properties similar to those of other proteins known to contain these domains.

TABLE 2H
Domain Analysis of NOV2

gnl|Pfam|pfam00026, asp, Eukaryotic aspartyl protease. Aspartyl (acid)
proteases include pepsins, cathepsins, and renins. Two-domain structure,
probably arising from ancestral duplication. This family does not include the
retroviral nor retrotransposon proteases (pfam00077), which are much smaller
and appear to be homologous to a single domain of the eukaryotic asp
proteases.
CD-Length = 376 residues, 99.5% aligned Score = 462 bits
1189), Expect = 2e−131

| | | | | |
|---|---|---|---|---|
| NOV 2: | 35 | KVPLIRKKSLRRTLSERGLLKDFLKKHNLNPARKYFPQWEAPTLVDEQPLENYLDMEYFG | 94 | (SEQ ID NO: 133) |
| | | ++\|\| + \|\|\| \|\|\|+\|+\| \|\|\| \| \| +\| + \| \|\| \|\|\|\| \|\|+\| | | |
| Sbjct: | 3 | RIPLKKVPSLREKLSEKGVLLDFLVKRKYEPTKKLTGGASSSRSAVE-PLLNYLDAEYYG | 61 | (SEQ ID NO: 134) |
| NOV 2: | 95 | TIGIGTPAQDFTVLFDTGSSNLWVPSVYCSSL-ACTNHNRFNPEDSSTYQATSETVSITY | 153 | |
| | | \|\| \|\|\|\| \| \|\|\|+\|\|\|\|\|\|+\|\|\|\|\|\|\|\|+\| \|\| \| \|+\| \|\|\|\|+ \| \|\|+\| | | |
| Sbjct: | 62 | TISIGTPPQKFTVVFDTGSSDLWVPSVYCTSSYACKGHGTFDPSKSSTYKNLGTTFSISY | 121 | |
| NOV 2: | 154 | GTGS-MTGILGYDTVQVGGISDTNQIFGLSETEPGSFLYYAPFDGILGLAYPSISSSGA- | 211 | |
| | | \| \|\| +\| \|\| \|\|\| \|\|\|\|+ \|\|\| \|\|\|+ \|\|\|\|\| \| \|\|\|\|\|\|\| +\|\|\| + \| | | |
| Sbjct: | 122 | GDGSSASGFLGQDTVTVGGITVTNQQFGLATKEPGSFFATAVFDGILGLGFPSIEAGGPY | 181 | |
| NOV 2: | 212 | TPVFDNINNQGLVSQDLFSVYLSADDQSGSVVIFGGIDSSYYTGSLNWVPVTVEGYWQIT | 271 | |
| | | \|\|\|\|\|\|+ +\|\|\|+ \|\|\|\|\|++\| +\| +\|\|\|\|+\| \| \|\|\|\|\| \|\|\|\|\| +\|\|\|\|\|\| | | |
| Sbjct: | 182 | TPVFDNLKSQGLIDSPAFSVYLNSDSGAGGEIIFGGVDPSKYTGSLTWVPVTSQGYWQIT | 241 | |
| NOV 2: | 272 | VDSITMNGEAIACAEGCQAIVDTGTSLLTGPTSPIANIQSDIGASENSD-GDMVVSCSAI | 330 | |
| | | +\|\|\|\|+ \| \|+ \|\|\|\|\|+\|\|\|\|\|\|\| \|\|\|\| ++ \| +\|\|\| + \|+ \|+ \| +\| | | |
| Sbjct: | 242 | LDSITVGGSTTFCSSGCQAILDTGTSLLYGPTSIVSKIAKAVGASLSEYSGEYVIDCDSI | 301 | |
| NOV 2: | 331 | SSLPDIVFTINGVQYPVPPSAYILQSEGS----CISGFQGMNLPTESGELWILGDVFIRQ | 386 | |
| | | \|\|\|\|\|\| \| \| \| + \|\|\|\|\|\|+\|\| \|+\|\|\|\| ++\| \| \|\|\|\|\|\|\|\|+\| | | |
| Sbjct: | 302 | SSLPDITFFIGGAKITVPPSAYVLQPSSGGSDICLSGFQSDDIPG--GPLWILGDVFLRS | 359 | |
| NOV 2: | 387 | YFTVFDRANNQVSLAPV 403 | | |
| | | + \|\|\|\| \|\|++ \|\|\| | | |
| Sbjct: | 360 | AYVVFDRDNNRIGLAPA 376 | | |

Pepsin is one of the main proteolytic enzymes secreted by the gastric mucosa. It consists of a single polypeptide chain and arises from its precursor, pepsinogen, by removal of a 41-amino acid segment from the amino end. Pepsin is particularly effective in cleaving peptide bonds involving aromatic amino acids. Samloff and Townes (1970) showed that the pepsinogen-5 derived from the stomach and excreted in the urine is absent in some persons. Family and population data supported the view that absence of PG-5 is recessive, i.e., persons with the PG-5 band on electrophoresis are either homozygous or heterozygous for a particular allele. Samloffet al. (1973) found no instance of absent PG-5 among Japanese, Chinese and Filipinos. Among American whites and blacks a frequency of 14% was found. Data, suggestive but not conclusive, of linkage of Kell (110900) and pepsinogen were reported by Weitkamp et al. (1975). Data of Gedde-Dahl et al. (1978) cast doubt on the linkage of PG and HLA. Whittington et al. (1980) excluded linkage of PG with either HLA or glyoxalase I. Korsnes et al. (1980) found no clear evidence of linkage between PG5 and 28 marker loci. Linkage below 25% recombination for HLA and GPT was ruled out. Linkage below 20% recombination was ruled out for Rh, PGM-1, and several others. The possibility of loose linkages included PgS-C6 and Pg5-MNSs. In the mouse, Szymura and Klein (1981) found linkage of urinary pepsinogen with the major histocompatibility complex. Arguing from homology, one might take this as suggestive evidence that a pepsinogen gene is on chromosome 6. See duodenal ulcer, hyperpepsinogenemic I (126850).

Sogawa et al. (1983) isolated a recombinant clone for the human pepsinogen gene by screening the Maniatis library of human genomic DNA with a swine pepsinogen cDNA as a probe. They concluded that the pepsinogen gene occupies about 9.4 kb pairs of genomic DNA and is separated into 9 exons by 8 introns of variable lengths. The predicted amino acid sequence of human pepsinogen consists of 373 residues and is 82% homologous with that of swine pepsinogen. The predicted sequence contains 15 amino acid residues at the NH₂ end, showing that the protein is synthesized as a prepepsinogen. In human gastric mucosa, 2 immunologically distinct classes of pepsinogen are synthesized. PG1 is restricted to the corpus, while PG2 is found throughout the stomach as well as in the proximal duodenum. PG1 is found in serum and urine in a ratio of about 1 to 10. PG2 is present in serum and seminal fluid but only trace amounts are found in urine. Serum PG1 and PG2 apparently originate from the stomach in the main, because the levels are very low after gastrectomy. PG2 in seminal fluid probably originates from the prostate. Frants et al. (1984) proposed a new genetic model to explain the inheritance of the urinary pepsinogen (PGI) polymorphism. They proposed that each main fraction—3, 4, and 5—in the multibanded electrophoretic pattern is determined by its own specific gene, B, C and D, respectively. The relative intensities of the fractions are determined by gene copy numbers. According to this model the PG1 system is inherited as autosomal codominant haplotypes. Some critical families not explained by previous models were presented in support of the hypothesis. In a note added in proof, the authors reported the resolution of a workshop to use PGA and PGC in place of PG1 and PG2, respectively. In man, there are 2 related pepsinogen systems: PGA, formerly PG I, precursor of pepsin A (EC 3.4.23.1), and PGC, formerly PG II, precursor of pepsin C (EC 3.4.23.3).

Except for the autosomal inheritance of the PGA polymorphism, no definite data on the chromosomal localization of these genes were available until the mapping of pepsinogen A to chromosome 11 (Frants et al., 1985; Taggart et al., 1985). The polymorphism of PGA is due to variation in the number of genes in the centromere region of chromosome 11. Taggart et al. (1985) proposed that the PG1 isozymogens, Pg3, Pg4, and Pg5, are encoded by closely linked genes, PGA3 (169710), PGA4 (169720), and PGA5 (169730), and that their presence or absence in different haplotype combinations determines phenotypic variation of PG1. Taggart et al. (1985) used a pepsinogen cDNA probe with man-rodent somatic cell hybrids to show that the complex is on chromosome 11. By means of 3 different X; 11 translocations, they narrowed the assignment to 11p12-11q13. Frants et al. (1985) likewise mapped PGA to chromosome 11 (11pter-11q12). Nakai et al. (1986) assigned the pepsinogen genes to 11q13 by in situ hybridization. Kidd (1986) found that the pepsinogen cluster is about 20 cM on the centromeric side of the CAT locus (115500). Hayano et al. (1986) obtained a cosmid clone containing 2 PGA genes in a single insert. Restriction endonuclease mapping showed that the two have very similar but distinct structures and that they are closely linked. The close situation of genes of very similar structure probably facilitates unequal crossing-over, which accounts for a high frequency of haplotype variation in copy number of PGA genes (Taggart et al., 1985). Taggart et al. (1987) analyzed by Southern blot analysis of DNA from somatic cell hybrids the 3 most common PGA haplotypes and demonstrated the presence of 3 genes in the PGA-A haplotype (PGA3, PGA4, and PGA5); 2 genes in the B haplotype (PGA3 and PGA4); and 1 gene in the C haplotype (PGA4). This unusual polymorphism of genomic DNA encoding very similar proteins probably reflects recent evolution by gene duplication. Kishi and Yasuda (1987) identified a ‘new’ polymorphism. Evers et al. (1987) contributed to the understanding of the molecular basis for the heterogeneity of the PGA isozymogen pattern by studies at the DNA level in a pair of pepsinogen genes. They demonstrated a single nucleotide difference giving rise to a glu-to-lys substitution of the 43rd amino acid residue of the activation peptide, leading to a charge difference of the corresponding isozymogens. The substitution was in 1 of 2 tandem genes. Zelle et al. (1988) amplified on the hypothesis that the heterogeneity in pepsinogen A resides in the existence of a variable number of copies of PGA genes and different combinations of these genes. From restriction enzyme analysis of the cluster, they developed hypotheses for the creation of the variety of haplotypes through unequal but homologous crossing over. In the PGA gene quadruplet, for example, 4 genes are arranged in a highly ordered fashion in a head-to-tail orientation. Using the length in kilobases of the large polymorphic EcoRI fragment of the PGA genes, this quadruplet could be described as 15.0—12.0—12.0—16.6.

See, for example, Evers, et a., Hum. Genet. 77: 182-187, 1987. PubMed ID: 3115885; Frants, et al., Hum. Genet. 65: 385-390, 1984. PubMed ID: 6693125; Frants, et al., Cytogenet. Cell Genet. 40: 632 only, 1985; Gedde-Dahl, et al., Cytogenet. Cell Genet. 22: 301-303, 1978. PubMed ID: 752491; Hayano, et al., Biochem. Biophys. Res. Commun. 138: 289-296, 1986. PubMed ID: 3017318; Korsnes, et al. L.; Ann. Hum. Genet. 44: 185-194, 1980. PubMed ID: 7316469; Nakai, et al., Cytogenet. Cell Genet. 43: 215-217, 1986. PubMed ID: 3467902; Samloff, et al., Am. J. Hum. Genet. 25: 178-180, 1973. PubMed ID: 4689038; Sogawa, et al., J. Biol. Chem. 258: 5306-5311, 1983. PubMed ID: 6300126; Szymura and Klein, Immunogenetics 13: 267-271, 1981. PubMed ID: 7275224; Taggart, et al., Somat. Cell Molec. Genet. 13: 167-172, 1987. PubMed ID: 3031827; Taggart, et al., Proc. Nat. Acad. Sci. 82: 6240-6244, 1985. PubMed ID: 3862130; Weitkamp, et al., Cytogenet. Cell Genet. 14: 451-452, 1975; Weitkamp, et al., Am. J. Hum. Genet. 27: 486-491, 1975. PubMed ID: 1155457; Whittington, et al., Cytogenet. Cell Genet. 28: 145-150, 1980. PubMed ID: 7438789; and Zelle, et al., Hum. Genet. 78: 79-82, 1988. PubMed ID: 2892778.

The protein similarity information, expression pattern, cellular localization, and map location for the NOV2 proteins and nucleic acids disclosed herein suggest that these Pepsin A Precursor-like proteins may have important structural and/or physiological functions characteristic of the Pepsin A Precursor family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The novel nucleic acids and proteins of the invention have applications in the diagnosis and/or treatment of various diseases and disorders. For example, the compositions of the present invention will have efficacy for the treatment of patients suffering from: hypercalceimia, ulcers, cancer, as well as other diseases, disorders and conditions.

The novel NOV2 nucleic acids encoding the Pepsin A Precursor-like proteins of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 protein has multiple hydrophilic regions, each of which can be used as an immunogen. In one embodiment, a contemplated NOV2 epitope is from about amino acids 2 to 4. In another embodiment, a contemplated NOV2 epitope is from about amino acids 40 to 70. In alternative embodiments, contemplated NOV2 epitopes include from about amino acids 140 to 145, 160 to 163, 210 to 215, 240 to 245, 290 to 305, 340 to 342, 350 to 353 and 380 to 385.

NOV3

A disclosed NOV3 nucleic acid (designated as CuraGen Acc. No. CG56936-01), which encodes a novel Ribonuclease Pancreatic-like protein and includes the 479 nucleotide sequence (SEQ ID NO:13) shown in Table 3A. An open reading frame for the mature protein was identified beginning with an GGC codon at nucleotides 13-15 and ending with a TAG codon at nucleotides 474-476. Putative untranslated regions downstream from the termination codon and upstream from the initiation codon are underlined in Table 3A, and the start and stop codons are in bold letters.

TABLE 3A
NOV3 Nucleotide Sequence

(SEQ ID NO: 13)

AGGAAACTATCTGGCCTCAAGTCATCACAAGTGACAAGAACAAACCCCTCTGTGGGGGAATAGTGGTACCTGCAG
GCAGGGTATCTTGTGCCTTCAATGAGCTGACAGACTGTCATTTTGAACTTTGTCTCACTCTGAAAGCAGAAAATG
GCCGAAAGGTTTTGGCAAGCAACCTTCTTGGGAGAAATGCAAATACCATTGATTTTTCGAGGCCTCTCATGGATG
AAGACATGCTCCTTTTTACAAGTGTGGTCAGGTTCCCTGATAACTCTTTGTATGATCATGTGGTTGCAGTACCTT
GCAGGAACGGGAACGTCATTCTGAGGGTAGTCCACATGCAAGTGTTCTAAAGTTGACATCACTGCTTCATCATTC
ACCTCATTTTCCCAGAACAGAAGCACCAAGAAAATTATCACCATTGCCATTGAGAGAAGAGATCTCAGACTCGGG
AGCTGATCTTGAGTTATTTAACATAGCCA

The nucleic acid sequence of NOV3 maps to chromosome 14 and has no similarity on the DNA level to any known sequence.

A disclosed NOV3 polypeptide (SEQ ID NO:14) is 141 amino acid residues in length and is presented using the one-letter amino acid code in Table 3B. The SignalP, Psort and/or Hydropathy results predict that NOV3 has a signal peptide and is likely to be localized to the endoplasmic reticulum (membrane) with a certainty of 0.5500. In alternative embodiments, a NOV3 polypeptide is located to the lysosome (lumen) with a certainty of 0.1900, the endoplasmic reticulum (lumen) with a certainty of 0.1000, or the outside of the cell with a certainty of 0.1000. The SignalP predicts a likely cleavage site for a NOV3 peptide between amino acid positions 19 and 20, i.e. at the dash in the sequence VND-EA.

TABLE 3B
Encoded NOV3 Protein Sequence

(SEQ ID NO: 14)

MAMVIIFLVLLFWENEVNDEAVMSTLEHLHVDYPQNDVPVPARYCNHMIIQRVIREPDHTCKKEHVFIHERPRKI
NGICISPKKVACQNLSAIFCFQSETKFKMTVCQLIEGTRYPACRYHYSPTEGFVLVTCDDLRPDSF

The NOV3 amino acid sequence was found to have 39 of 134 amino acid residues (29%) identical to, and 69 of 134 amino acid residues (51%) similar to, the 156 amino acid residue ptnr:SWISSNEW-ACC:P07998 protein from Homo sapiens (Human) (RIBONUCLEASE PANCREATIC PRECURSOR (EC 3.1.27.5) (RNASE 1) (RNASE A) (RNASE UPI-1) (RIB-1)) (E=1.3e⁻¹³).

NOV3 is expressed in at least the following tissues: pancreas, lung, testis, and b-cell. Expression information was derived from the tissue sources of the sequences that were included in the derivation of the sequence of CuraGen Acc. No. CG56936-01.

Possible small nucleotide polymorphisms (SNPs) found for NOV3 are listed in Table 3C.

TABLE 3C
SNPs

| | Nucleotide | | Amino Acid | |
|---|---|---|---|---|
| Variant | Position | Base Change | Position | Base Change |
| 13376210 | 117 | T > C | NA | NA |
| 13376983 | 164 | C > T | 55 | Pro > Leu |
| 13376211 | 205 | A > G | 69 | Arg > Gly |
| 13376985 | 338 | A > G | 113 | Tyr > Cys |
| 13376986 | 354 | C > T | NA | NA |
| 13376987 | 371 | A > G | 124 | Glu > Gly |

NOV3 has homology to the amino acid sequences shown in the BLASTP data listed in Table 3D.

TABLE 3D
BLAST results for NOV3

| Gene Index/ | | Length | Identity | Positives | |
|---|---|---|---|---|---|
| Identifier | Protein/Organism | (aa) | (%) | (%) | Expect |
| gi\|12853968\| | Pancreatic | 208 | 37/107 | 59/107 | 6e−09 |
| dbjBAB29898.1\| | ribonucleases | | (34%) | (54%) | |
| (AK015573) | containing | | | | |
| | protein: Pfam, | | | | |
| | source | | | | |
| | key: PF00074, | | | | |
| | evidence: | | | | |
| | ISS-putative [Mus | | | | |
| | musculus] | | | | |
| gi\|13124491\|sp\| | Ribonuclease | 149 | 37/130 | 66/130 | 1e−08 |
| Q9QYX3\|RNP_{—} | pancreatic | | (28%) | (50%) | |
| MUSPA | precursor (RNase | | | | |
| | 1) (RNase A) | | | | |
| gi\|13399882\|pdb\| | Chain A, 3-D | 129 | 35/115 | 58/115 | 1e−08 |
| 1DZA\|A | Structure Of A | | (30%) | (50%) | |
| | Hp-Rnase | | | | |
| gi\|133226\|sp\| | RIBONUCLEASE | 128 | 31/91 | 51/91 | 1e−08 |
| P19644\|RNP_PREEN | PANCREATIC (RNASE | | (34%) | (55%) | |
| | 1) (RNASE A) | | | | |
| gi\|464659\|sp\| | RIBONUCLEASE | 119 | 32/118 | 58/118 | 1e−08 |
| P80287\|RNP_{—} | PANCREATIC (RNASE | | (27%) | (49%) | |
| IGUIG | 1) (RNASE A) | | | | |

The homology of these sequences is shown graphically in the ClustalW analysis shown in Table 3E.

[image]

[image]

Table 3F lists the domain description from DOMAIN analysis results against NOV3.

This indicates that the NOV3 sequence has properties similar to those of other proteins known to contain these domains.

TABLE 3F
Domain Analysis of NOV3

gnl|Smart|smart00092, RNAse_Pc, Pancreatic ribonuclease
CD-Length 123 residues, 80.5% aligned
Score = 68.2 bits (165), Expect = 3e−13

| | | | |
|---|---|---|---|
| NOV 3: | 30 | HVDYPQNDVPVPARYCNHMIIQRVIREPDHTCKKEHVFIHERPRKINGICISPKKVACQN 89 | (SEQ ID NO: 140) |
| | | \|+\| + \|\|\| \|+ +\|+ +\| + + \|\|+ \|+\|\| + +\| \| \| \| \|+\| | |
| Sbjct: | 12 | HIDSTPS--SASDNYCNQMMKRRNMTQ--GRCKPVNTFVHESLADVKAVC-SQKNVTCKN 66 | (SEQ ID NO: 141) |
| NOV 3: | 90 | LSAIFCFQSETKFKMTVCQLIEGTRYPACRYHYSPTEGFVLVTCD 134 | |
| | | \| \|\| ++\|++\| \|+\| \|++\|\| \|\|\| + ++\| \|+ | |
| Sbjct: | 67 | -GRTNCHQSNSRFQLTDCRLTGGSKYPNCRYKTTQANKHIIVACE 110 | |

gnl|Pfam|pfam00074, rnaseA, Pancreatic ribonuclease. Ribonucleases. Members
include pancreatic RNAase A and angiogenins. Structure is an alpha + beta fold-
long curved beta sheet and three helices.
CD-Length = 122 residues, 73.0% aligned
Score = 64.3 bits (155), Expect = 4e−12

| | | | |
|---|---|---|---|
| NOV 3: | 42 | ARYCNHMIIQRVIREPDHTCKKEHVFIHERPRKINGICISPKKVACQNLSAIFCFQSETK 101 | (SEQ ID NO: 142) |
| | | \|\|\| \|+ +\| + + \|\| + \|+\|\| + +\| \| \| \| \|+\| \|+\|\| + | |
| Sbjct: | 22 | DNYCNQMMKRRNMTQG--RCKPVNTFVHESLADVKAVC-SQKNVTCKNGQKN-CYQSTSS 77 | (SEQ ID NO: 143) |
| NOV 3: | 102 | FKMTVCQLIEGTRYPACRYNYSPTEGFVLVTCD 134 | |
| | | \|++\| \|+\| \|++\|\| \|\|\| +\| ++\| \|+ | |
| Sbjct: | 78 | FQLTDCRLTGGSKYPNCRYRTTPGNKRIIVACE 110 | |

Pancreatic ribonuclease (EC 3.1.27.5) is one of the digestive enzymes secreted in abundance by the pancreas. Elliott et al. (Cytogenet. Cell Genet. 42: 110-112, 1986) mapped the mouse gene to chromosome 14 by Southern blot analysis of genomic DNA from recombinant inbred strains of mice, using a probe isolated from a pancreatic cDNA library with the rat cDNA. The assignment to mouse 14 and the close linkage to the other 2 loci was confirmed by study of one of Snell's congenic strains: the 3 loci went together. Elliott et al. (Cytogenet. Cell Genet. 42: 110-112, 1986) predicted that the homologous human gene RIB1 is on chromosome 14.

Human pancreatic RNase is monomeric and is devoid of any biologic activity other than its RNA degrading ability. Piccoli et al. (Proc. Nat. Acad. Sci. 96: 7768-7773, 1999) engineered the monomeric form into a dimeric protein with cytotoxic action on mouse and human tumor cells, but lacking any appreciable toxicity on human and mouse normal cells. The dimeric variant of human pancreatic RNase selectively sensitized cells derived from a human thyroid tumor to apoptotic death. Because of its selectivity for tumor cells, and because of its human origin, this protein was thought to represent an attractive tool for anticancer therapy.

The protein similarity information, expression pattern, cellular localization, and map location for the NOV3 protein and nucleic acid disclosed herein suggest that this ribonuclease pancreatic-like protein may have important structural and/or physiological functions characteristic of the Ribonuclease Pancreatic family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from cancer as well as other diseases, disorders and conditions.

The novel nucleic acid encoding the Ribonuclease Pancreatic-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV3 protein has multiple hydrophilic regions, each of which can be used as an immunogen. In one embodiment, a contemplated NOV3 epitope is from about amino acids 20 to 30. In another embodiment, a contemplated NOV3 epitope is from about amino acids 35 to 42. In other specific embodiments, contemplated NOV3 epitopes are from about amino acids 52 to 55, 60 to 70, 70 to 72, 110 to 115, 118 to 124 and 130 to 135.

NOV4 and NOV5

This invention includes two novel Ser/Tbr kinase-like proteins. The disclosed proteins have been named NOV4 and NOV5.

NOV4

A disclosed NOV4 nucleic acid (designated as CG51707-02), encodes a novel Serfrhr Kinase-like protein and includes the 1037 nucleotide sequence (SEQ ID NO:15) shown in Table 4A. An open reading frame for the mature protein was identified beginning with an ATG codon at nucleotides 41-43 and ending with a TGA codon at nucleotides 1019-1021. Putative untranslated regions downstream from the termination codon and upstream from the initiation codon are underlined in Table 4A, and the start and stop codons are in bold letters.

TABLE 4A
NOV4 Nucleotide Sequence

(SEQ ID NO: 15)

GCGCCGCGTGGGGGACGGAAGTGAAACTCTAAGAAATGAGATGGAGAAGTACGAGCGGATCCGAGTGGTGGGGAGA
GGTGCCTTCGGGATTGTGCACCTGTGCCTGCGAAAGGCTGACCAGAAGCTGGTGATCATCAAGCAGATTCCAGTGG
AACAGATGACCAAGGAAGAGCGGCAGGCAGCCCAGAATGAGTGCCAGGTCCTCAAGCTGCTCAACCACCCCAATGT
CATTGAGTACTACGAGAACTTCCTGGAAGACAAAGCCCTTATGACCGCCATGGAATATGCACCAGGCGGCACTCTG
GCTGAGTTCATCCAAAAGCGCTGTAATTCCCTGCTGGAGGAGGAGACCATCCTGCACTTCTTCGTGCAGATCCTGC
TTGCACTGCATCATGTGCACACCCACCTCATCCTGCACCGAGACCTCAAGACCCAGAACATCCTGCTTGACAAACA
CCGCATGGTCGTCAAGATCGGTGATTTCGGCATCTCCAAGATCCTTAGCAGCAAGAGCAAGGCCTACACGGTGGTG
GGTACCCCATGCTATATCTCCCCTGAGCTGTGTGAGGGCAAGCCCTACAACCAGAAGAGTGACATCTGGGCCCTGG
GCTGTGTCCTCTACGAGCTGGCCAGCCTCAAGAGGGCTTTCGAGGCTGCGAACTTGCCAGCACTGGTGCTGAAGAT
CATGAGTGGCACCTTTGCACCTATCTCTGACCGGTACAGCCCTGAGCTTCGCCAGCTGGTCCTGAGTCTACTCAGC
CTGGAGCCTGCCCAGCGGCCACCACTCAGCCACATCATGGCACAGCCCCTCTGCATCCGTGCCCTCCTCAACCTCC
ACACCGACGTGGGCAGTGTCCGCATGCGGAGGCCTGTGCAGGGACAGCGAGCGGTCCTGGGCGGCAGGGTGTGGGC
ACCCAGTGGGAGCACACTTTCGCCTCTGACTGTGTCCGCCACAGCCTGCACCTACACTCTGTCATCTTTTACCATT
GACACCTTGCACCATGATCTGAAAACACAATGACTTAGTCATCTGCCAA

The nucleic acid sequence of NOV4 maps to chromosome 17 has 463 of 759 bases (61%) identical to a gb:GENBANK-ID:AF087909|acc:AF087909.1 mRNA from Homo sapiens (Homo sapiens NIMA-related kinase 6 (NEK6) mRNA, complete cds) (E=1.9e⁻²³).

The NOV4 polypeptide (SEQ ID NO:16) is 326 amino acid residues in length and is presented using the one-letter amino acid code in Table 4B. The SignalP, Psort and/or Hydropathy results predict that NOV4 does not have a signal peptide and is likely to be localized to the cytoplasm with a certainty of 0.6500. In alternative embodiments, a NOV4 polypeptide is located to the lysosome (lumen) with a certainty of 0.1866 or the mitochondrial matrix space with a certainty of 0.1000.

TABLE 4B
Encoded NOV4 Protein Sequence

(SEQ ID NO:16)

MEKYERIRVVGRGAFGIVHLCLRKADQKLVIIKQIPVEQMTKEERQAAQNECQVLKLLNHPNVIEYYENFLEDK
ALMTAMEYAPGGTLAEFIQKRCNSLLEEETILHFFVQILLALHHVHTHLILHRDLKTQNILLDKHRMVVKIGDF
GISKILSSKSKAYTVVGTPCYISPELCEGKPYNQKSDIWALGCVLYELASLKRAFEAANLPALVLKIMSGTFAP
ISDRYSPELRQLVLSLLSLEPAQRPPLSHIMAQPLCIRALLNLHTDVGSVRMRRPVQGQRAVLGGRVWAPSGST
LSPLTVSATACTYTLSSFTIDTLHHDLKTQ

The NOV4 amino acid sequence was found to have 152 of 333 amino acid residues (45%) identical to, and 218 of 333 amino acid residues (65%) similar to, the 357 amino acid residue ptnr:SPTREMBL-ACC:001775 protein from Caenorhabditis elegans (SIMILARITY TO THE CDC2/CDX SUBFAMILY OF SER/THR PROTEIN KINASES) (E=1.6e⁻⁶⁸).

NOV4 is expressed in at least the following tissues: fetal lung, other developmental tissues, germ cells and sex tissues. Expression information was derived from the tissue sources of the sequences that were included in the derivation of the sequence of NOV4.

Possible small nucleotide polymorphisms (SNPs) found for NOV4 are listed in Table 4C.

TABLE 4C
SNPs

| | Nucleotide | | Amino Acid | |
|---|---|---|---|---|
| Variant | Position | Base Change | Position | Base Change |
| 13376988 | 105 | T > G | 22 | Leu > Arg |
| 13376989 | 204 | T > C | 55 | Leu > Pro |
| 13376990 | 368 | G > A | 110 | Val > Met |
| 13376991 | 712 | T > C | NA | NA |

NOV4 also has homology to the amino acid sequences shown in the BLASTP data listed in Table 4D.

TABLE 4D
BLAST results for NOV4

| Gene Index/ | | Length | Identity | Positives | |
|---|---|---|---|---|---|
| Identifier | Protein/Organism | (aa) | (%) | (%) | Expect |
| gi\|15825377\|gb\| | NIMA-related | 698 | 273/276 | 275/276 | e−157 |
| AAL09675.1\| | kinase 8 [Mus | | (98%) | (98%) | |
| AF407579_1 | musculus] | | | | |
| (AF407579) | | | | | |
| gi\|12852471\|dbj\| | data source: SPTR, | 291 | 275/280 | 277/280 | e−155 |
| BAB29424.1\| | source key: P51954, | | (98%) | (98%) | |
| (AK014546) | evidence: ISS˜puta- | | | | |
| | tive˜similar to | | | | |
| | SERINE/THREONINE- | | | | |
| | PROTEIN KINASE | | | | |
| | NEK1 (EC 2.7.1.-) | | | | |
| | (NIMA-RELATED | | | | |
| | PROTEIN KINASE 1) | | | | |
| | [Mus musculus] | | | | |
| gi\|15825379\|gb\| | NIMA-related | 697 | 242/323 | 276/323 | e−138 |
| AAL09676.1\| | kinase 8 [Danio | | (74%) | (84%) | |
| AF407580_1 | rerio] | | | | |
| (AF407580) | | | | | |
| gi\|17511015\|ref\| | ser/thr-protein | 357 | 148/335 | 212/335 | 3e−71 |
| NP_491914.1\| | kinase | | (44%) | (63%) | |
| (NM_059513) | [Caenorhabditis | | | | |
| | elegans] | | | | |
| gi\|7301213\|gb\| | CG10951 gene | 841 | 125/265 | 177/265 | 2e−64 |
| AAF56344.1\| | product | | (47%) | (66%) | |
| (AE003749) | [Drosophila | | | | |
| | melanogaster] | | | | |

The homology of these sequences is shown graphically in the ClustalW analysis shown in Table 4E.

[image]

[image]

[image]

Tables 4F-G list the domain description from DOMAIN analysis results against NOV4. This indicates that the NOV4 sequence has properties similar to those of other proteins known to contain these domains.

TABLE 4F

Domain Analysis of NOV4

gnl|Smart|smart00220, S_TKc, Serine/Threonine protein kinases, catalytic
domain; Phosphotransferases. Serine or threonine-specific kinase subfamily.
CD-Length = 256 residues, 99.2% aligned
Score = 223 bits (567), Expect = 2e−59

| | | | | |
|---|---|---|---|---|
| NOV 4: | 4 | YERIRVVGRGAFGIVHLCLRKADQKLVIIKQIPVEQMTKEERQAAQNECQVLKLLNHPNV | 63 | (SEQ ID NO:149) |
| | | \|\| + \|+\|+\|\|\|\| \|+\| \| \|\|\| \|\| \| \|++ \|++\|+ \| ++\|\| \|+\|\|\|+ | | |
| Sbjct: | 1 | YELLEVLGKGAFGKVYLARDKKTGKLVAIKVIKKEKLKKKKRERILREIKILKKLDHPNI | 60 | (SEQ ID NO:150) |
| NOV 4: | 64 | IEYYENFLEDKALMTAMEYAPGGTLAEFIQKRCNSLLEEETILHFFVQILLALHHVHTHL | 123 | |
| | | ++ \|+ \| +\| \| \|\|\| \|\| \| + ++\|\| \| \|+ + \|\|\| \|\| ++\|+ | | |
| Sbjct: | 61 | VKLYDVFEDDDKLYLVMEYCEGGDLFDLLKKRGR--LSEDEARFYARQILSALEYLHSQG | 118 | |
| NOV 4: | 124 | ILHRDLKTQNILLDKHRMVVKIGDFGISKILSSKS-KAYTVVGTPCYISPELCEGKPYNQ | 182 | |
| | | \|+\|\|\|\|\| +\|\|\|\|\| \|\|+ \|\|\|++\| \| \| \| \|\|\|\| \|++\|\|+ \|\| \| + | | |
| Sbjct: | 119 | IIHRDLKPENILLDSD-GHVKLADFGLAKQLDSGGTLLTTFVGTPEYMAPEVLLGKGYGK | 177 | |
| NOV 4: | 183 | KSDIWALGCVLYELASLKRAFEAANLPALVLKIMSG---TFAPISDRYSPELRQLVLSLL | 239 | |
| | | \|\|\|+\|\| +\|\|\|\| + \| \| + + \| + \| \| + \|\|\| + \|+ \|\| | | |
| Sbjct: | 178 | AVDIWSLGVILYELLTGKPPFPGDDQLLALFKKIGKPPPPFPPPEWKISPEAKDLIKKLL | 237 | |
| NOV 4: | 240 | SLEPAQRPPLSHIMAQP 256 | | |
| | | +\| +\| + \| | | |
| Sbjct: | 238 | VKDPEKRLTAEEALEHP 254 | | |

TABLE 4G

Domain Analysis of NOV4

gnl|Pfam|pfam00069, pkinase, Protein kinase domain.
CD-Length = 256 residues, 99.2% aligned
Score = 209 bits (533), Expect = 2e−55

| | | | | |
|---|---|---|---|---|
| NOV 4: | 4 | YERIRVVGRGAFGIVHLCLRKADQKLVIIKQIPVEQMTKEERQAAQNECQVLKLLNHPNV | 63 | (SEQ ID NO:149) |
| | | \|\| +\| \|\|\|\| \|+ \| ++\| \|\| + + \|+++ \| \|+\|+ \|+\|\|\|+ | | |
| Sbjct: | 1 | YELGEKLGSGAFGKVYKGKHKDTGEIVAIKILKKRSL-SEKKKRFLREIQILRRLSHPNI | 59 | (SEQ ID NO:151) |
| NOV 4: | 64 | IEYYENFLEDKALMTAMEYAPGGTLAEFIQKRCNSLLEEETILHFFVQILLALHHVHTHL | 123 | |
| | | + \| \|\| \| \|\|\| \|\| \| ++++ \| \|\| \|+ +\|\|\| \| ++\|+ | | |
| Sbjct: | 60 | VRLLGVFEEDDHLYLVMEYMEGGDLFDYLR-RNGLLLSEKEAKKIALQILRGLEYLHSRG | 118 | |
| NOV4: | 124 | ILHRDLKTQNILLDKHRMVVKIGDFGISKILSSK--SKAYTVVGTPCYISPELCEGKPYN | 181 | |
| | | \|+\|\|\|\|\| +\|\|\|\|\|++ \|\|\| \|\|\|+++ \| \| \| \| \|\|\|\| \|++\|\|+ \|\|+ \|+ | | |
| Sbjct: | 119 | IVHRDLKPENILLDEN-GTVKIADFGLARKLESSSYEKLTTFVGTPEYMAPEVLEGRGYS | 177 | |
| NOV 4: | 182 | QKSDIWALGCVLYELASLKRAFEAANLPALVLKIMSGTF--APISDRYSPELRQLVLSLL | 239 | |
| | | \| \|+\|+\|\| +\|\|\|\| + \| \| + + +\| \|+ \| \|\|+ \|+ \| | | |
| Sbjct: | 178 | SKVDVWSLGVILYELLTGKLPFPGIDPLEELFRIKERPRLRLPLPPNCSEELKDLIKKCL | 237 | |
| NOV 4: | 240 | SLEPAQRPPLSHIMAQP 256 | | |
| | | + +\| +\|\| \|+ \| | | |
| Sbjct: | 238 | NKDPEKRPTAKEILNHP 254 | | |

TABLE 4H

Domain Analysis of NOV4

gnl|Smart|smart00219, TyrKc, Tyrosine kinase, catalytic domain;
Phosphotransferases. Tyrosine-specific kinase subfamily.
CD-Length = 258 residues, 96.9% aligned
Score = 136 bits (343), Expect = 2e−33

| | | | | |
|---|---|---|---|---|
| NOV 4: | 8 | RVVGRGAFGIVHLCL---RKADQKLVIIKQIPVEQMTKEERQAAQNECQVLKLLNHPNVI | 64 | (SEQ ID NO:152) |
| | | + +\| \|\|\|\| \|+ + + \| +\| + + ++ + \| ++++ \|+\|\|\|++ | | |
| Sbjct: | 5 | KKLGEGAFGEVYKGTLKGKGGVEVEVAVKTLKEDASEQQ-IEEFLREARLMRKLDHPNIV | 63 | (SEQ ID NO:153) |
| NOV 4: | 65 | EYYENFLEDKALMTAMEYAPGGTLAEFIQKRCNSLLEEETILHFFVQILLALHHVHTHLI | 124 | |
| | | + \|++ \|\| \|\|\| \|\| \| ++++\| \| +\| \| +\|\| + ++ + | | |
| Sbjct: | 64 | KLLGVCTEEEPLMIVMEYMEGGDLLDYLRKNRPKELSLSDLLSFALQIARGMEYLESKNF | 123 | |
| NOV 4: | 125 | LHRDLKTQNILLDKHRMVVKIGDFGISKILSSKSKAYTVVGTPC----YISPELCEGKPY | 180 | |
| | | +\|\|\|\| +\| \|+ +++ \|\|\| \|\|\|+++ \| \| +\| +++\|\| + + | | |
| Sbjct: | 124 | VHRDLAARNCLVGENK-TVKIADFGLARDLYDD-DYYRKKKSPRLPIRWMAPESLKDGKF | 181 | |
| NOV 4: | 181 | NQKSDIWALGCVLYELASL-KRAFEAANLPALVLKIMSGTFAPISDRYSPELRQLVLSLL | 239 | |
| | | \|\|\|+\|+ \| +\|+\|+ +\| + + + ++ + \| \| \|+ \|+\| | | |
| Sbjct: | 182 | TSKSDVWSFGVLLWEIFTLGESPYPGMSNEEVLEYLKKGYRLPQPPNCPDEIYDLMLQCW | 241 | |
| NOV 4: | 240 | SLEPAQRPPLSHI 252 | | |
| | | + +\| \|\| \| + | | |
| Sbjct: | 242 | AEDPEDRPTFSEL 254 | | |

NOV5

A disclosed NOV5 nucleic acid (designated as CG57081-01) includes the 1591 nucleotide sequence (SEQ ID NO:17) shown in Table 5A. An open reading frame for the mature protein was identified beginning with an ATG codon at nucleotides 31-33 and ending with a TAG codon at nucleotides 1495-1497. The start and stop codons of the open reading frame are highlighted in bold type. Putative untranslated regions are underlined and found upstream from the initiation codon and downstream from the termination codon.

TABLE 5A
NOV5 Nucleotide Sequence

(SEQ ID NO:17)

TCCGGCTGCCGCGCGCACCCAGACCCGGCGATGAGGAGTGGCGCCGAGCGCAGGGGCAGCAGCGCCGCGGCGTC
CCCGGGCTCGCCGCCCCCCGGCCGCGCGCGCCCCGCCGGCTCCGACGCGCCCTCGGCCCTGCCGCCGCCCGCTG
CTGGCCAGCCCCGGGCCCGGGACTCGGGCGATGTCCGCTCGCAGCCGCGCCCCCTGTTTCAGTGGAGCAAGTGG
AAGAAGAGGATGGGCTCGTCCATGTCGGCGGCCACCGCGCGGAGGCCGGTGTTTGACGACAAGGAGGACGTGAA
CTTCGACCACTTCCAGATCCTTCGGGCCATTGGGAAGGGCAGCTTTGGCAAGGTAGTGTGCATTGTGCAGAAGC
GGGACACGGAGAAGATGTACGCCATGAAGTACATGAACAAGCAGCAGTGCATCGAGCGCGACGAGGTCCGGAAT
GTCTTCCGGGAGCTGGAGATCCTGCAGGAGATCGAGCATGTCTTCCTGGTGAACCTCTGGTATTCATTCCAAGA
TGAGGAGGACATGTTCATGGTGGTAGACCTGCTTCTGGGTGGAGACCTACGTTACCACCTGCAGCAGAACGTGC
AGTTCTCCGAGGACACAGTGAGGCTGTACATCTGCGAGATGGCACTGGCTCTGGACTACCTGCGCGGCCAGCAC
ATCATCCACAGAGATGTCAAGCCTGACAACATTCTCCTGGATGAGAGAGGACATGCACACCTGACCGACTTCAA
CATTGCCACCATCATCAAGGACGGGGAGCGGGCGACGGCATTAGCAGGCACCAAGCCGTACATGGCTCCGGAGA
TCTTCCACTCTTTTGTCAACGGCGGGACCGGCTACTCCTTCGAGGTGGACTGGTGGTCGGTGGGGGTGATGGCC
TATGAGCTGCTGCGAGGATGGAGGCCCTATGACATCCACTCCAGCAACGCCGTGGAGTCCCTGGTGCAGCTGTT
CAGCACCGTGAGCGTCCAGTATGTCCCCACGTGGTCCAAGGAGATGGTGGGCTTGCTGCGGAAGGTGCTCCTCA
CTGTGAACCCCGAGCACCGGCTCTCCAGCCTCCAGGACGTGCAGGCAGCCCCGGCGCTGGCCGGCGTGCTGTGG
GACCACCTGAGCGAGAAGAGGGTGGAGCCGGGCTTCGTGCCCAACAAAGGCCGTCTGCACTGCGACCCCACCTT
TGAGCTGGAGGAGATGATCCTGGAGTCCAGGCCCCTGCACAAGAAGAAGAAGCGCCTGGCCAAGAACAAGTCCC
GGGACAACAGCAGGGACAGCTCCCAGTCCGAGAATGACTATCTTCAAGACTGCCTCGATGCCATCCAGCAAGAC
TTCGTGATTTTTAACAGAGAAAAGCTGAAGAGGAGCCAGGACCTCCCGAGGGAGCCTCTCCCCGCCCCTGAGTC
CAGGGATGCTGCGGAGCCTGTGGAGGACGAGGCGGAACGCTCCGCCCTGCCCATGTGCGGCCCCATTTGCCCCT
CGGCCGGGAGCGGCTAGGCCGGGACGCCCGTGGTCCTCACCCCTTGAGCTGCTTTGGAGACTCGGCTGCCAGAG
GGAGGGCCATGGGCCGAGGCCTGGCATTCACGTTCCC

The nucleic acid sequence of NOV5 maps to chromosome 10 and has 1338 of 1549 bases (86%) identical to a gb:GENBANK-ID:AB041542|acc:AB041542.1 mRNA from Mus musculus (Mus musculus brain cDNA, clone MNCb-1563, similar to AJ250840 serine/threonine protein kinase (Mus musculus)) (E=1.9e⁻²⁵¹).

A disclosed NOV5 polypeptide (SEQ ID NO:18) is 488 amino acid residues and is presented using the one letter code in Table 5B. Signal P, Psort and/or Hydropathy results predict that NOV5 does not have a signal peptide and is likely to be localized to the nucleus with a certainty of 0.7000. In other embodiments, NOV5 is localized to the microbody (peroxisome) with a certainty of 0.3058, the mitochondrial matrix space with a certainty of 0.1000 or the lysosome (lumen) with a certainty of 0.1000.

TABLE 5B
Encoded NOV5 Protein Sequence

(SEQ ID NO:18)

MRSGAERRGSSAAASPGSPPPGRARPAGSDAPSALPPPAAGQPRARDSGDVRSQPRPLFQWSKWKKRMGSSMSA
ATARRPVFDDKEDVNFDHFQILRAIGKGSFGKVVCIVQKRDTEKMYAMKYMNKQQCIERDEVRNVFRELEILQE
IEHVFLVNLWYSFQDEEDMFMVVDLLLGGDLRYHLQQNVQFSEDTVRLYICEMALALDYLRGQHIIHRDVKPDN
ILLDERGHAHLTDFNIATIIKDGERATALAGTKPYMAPEIFHSFVNGGTGYSFEVDWWSVGVMAYELLRGWRPY
DIHSSNAVESLVQLFSTVSVQYVPTWSKEMVGLLRKVLLTVNPEHRLSSLQDVQAAPALAGVLWDHLSEKRVEP
GFVPNKGRLHCDPTFELEEMILESRPLHKKKKRLAKNKSRDNSRDSSQSENDYLQDCLDAIQQDFVIFNREKLK
RSQDLPREPLPAPESRDAAEPVEDEAERSALPMCGPICPSAGSG

The NOV5 amino acid sequence was found to have 442 of 487 amino acid residues (90%) identical to, and 458 of 487 amino acid residues (94%) similar to, the 488 amino acid residue ptnr:SPTREMBL-ACC:Q9JJG4 protein from Mus musculus (Mouse) (BRAIN cDNA, CLONE MNCB-1563, SIMILAR TO AJ250840 SERINE/THREONINE PROTEIN

KINASE (MUS MUSCULUS)) (E=1.1e⁻²³⁸).

NOV5 is expressed in at least the following tissues: brain, kidney, liver, pancreas, peripheral blood, prostate, testis, thalamus, thymus, uterus, lymph node, lymphoid tissue, bone marrow, and spleen. Expression information was derived from the tissue sources of the sequences that were included in the derivation of the sequence of NOV5. The sequence is predicted to be expressed in the following tissues because of the expression pattern of (GENBANK-ID: gb:GENBANK-ID:AB041542|acc:AB041542.1) a closely related Mus musculus brain cDNA, clone MNCb-1563, similar to AJ250840 serine/threonine protein kinase (Mus musculus) homolog in species Mus musculus: brain.

NOV5 also has homology to the amino acid sequences shown in the BLASTP data listed in Table 5C.

TABLE 5C
BLAST results for NOV5

| Gene Index/ | | Length | Identity | Positives | |
|---|---|---|---|---|---|
| Identifier | Protein/Organism | (aa) | (%) | (%) | Expect |
| gi\|10946600\|ref\| | hypothetical | 488 | 441/489 | 457/489 | 0.0 |
| NP_067277.1\| | serine/threonine | | (90%) | (93%) | |
| (NM_021302) | protein kinase | | | | |
| | [Mus musculus] | | | | |
| gi\|17453579\|ref\| | similar to | 369 | 368/370 | 368/370 | 0.0 |
| XP_058348.1\| | Unknown (protein | | (99%) | (99%) | |
| (XM_058348) | for MGC: 23665) | | | | |
| | (H. sapiens) | | | | |
| | [Homo sapiens] | | | | |
| gi\|13358640\|dbj\| | hypothetical | 368 | 357/370 | 360/370 | 0.0 |
| BAB33045.1\|(AB056389) | protein [Macaca | | (96%) | (96%) | |
| | fascicularis] | | | | |
| gi\|8923754\|ref\| | gene for | 414 | 261/368 | 314/368 | e−161 |
| NP_060871.1\| | serine/threonine | | (70%) | (84%) | |
| (NM_018401) | protein kinase | | | | |
| | [Homo sapiens] | | | | |
| gi\|7161864\|emb\| | serine/threonine | 414 | 260/368 | 317/368 | e−160 |
| CAB76566.1\|(AJ250840) | protein kinase | | (70%) | (85%) | |
| | [Mus musculus] | | | | |

The homology of these sequences is shown graphically in the ClustalW analysis shown in Table 5H.

[image]

[image]

Tables 5E-G list the domain description from DOMAIN analysis results against NOV5. This indicates that the NOV5 sequence has properties similar to those of other proteins known to contain these domains.

TABLE 5E

Domain Analysis of NOV5

gnl|Smart|smart00220, S_TKc, Serine/Threonine protein kinases, catalytic
domain; Phosphotransferases. Serine or threonine-specific kinase subfamily.
CD-Length = 256 residues, 98.4% aligned
Score = 230 bits (587), Expect = 1e−61

| | | | | |
|---|---|---|---|---|
| NOV 5: | 93 | FQILRAIGKGSFGKVVCIVQKRDTEKMYAMKYMNKQQCIERDEVRNVFRELEILQEIEHV | 152 | (SEQ ID NO:159) |
| | | +++\| +\|\|\|+\|\|\|\| + + \| \|+ \|+\| + \|++ +++ + + \|\|++\|\|++++\| | | |
| Sbjct: | 1 | YELLEVLGKGAFGKVYL-ARDKKTGKLVAIKVIKKEK-LKKKKRERILREIKILKKLDHP | 58 | (SEQ ID NO:160) |
| NOV 5: | 153 | FLVNLWYSFQDEEDMFMVVDLLLGGDLRYHLQQNVQFSEDTVRLYICEMALALDYLRGQH | 212 | |
| | | +\| \|+ \|+\|++ +++\|++ \|\|\|\| \|++ + \|\|\| \| \| ++ \|\|+\|\| \| | | |
| Sbjct: | 59 | NIVKLYDVFEDDDKLYLVMEYCEGGDLFDLLKKRGRLSEDEARFYARQILSALEYLHSQG | 118 | |
| NOV 5: | 213 | IIHRDVKPDNILLDERGHAHLTDFNIATIIKDG-ERATALAGTKPYMAPEIFHSFVNGGT | 271 | |
| | | \|\|\|\|\|+\|\|+\|\|\|\|\| \|\| \| \|\| +\| + \| \| \|\| \|\|\|\|\|+ \| | | |
| Sbjct: | 119 | IIHRDLKPENILLDSDGHVKLADFGLAKQLDSGGTLLTTFVGTPEYMAPEVLL-----GK | 173 | |
| NOV 5: | 272 | GYSFEVDWWSVGVMAYELLRGWRPYDIHSS-NAVESLVQLFSTVSVQYVPTWSKEMVGLL | 330 | |
| | | \|\| \|\| \|\|+\|\|+ \|\|\|\| \| \|+ \|+ + \| \| \|+ | | |
| Sbjct: | 174 | GYGKAVDIWSLGVILYELLTGKPPFPGDDQLLALFKKIGKPPPPFPPPEWKISPEAKDLI | 233 | |
| NOV 5: | 331 | RKVLLTVNPEHRLSSLQDVQ 350 | | |
| | | +\| \|\| +\|\| \|\|++ + ++ | | |
| Sbjct: | 234 | KK-LLVKDPEKRLTAEEALE 252 | | |

TABLE 5F

Domain Analysis of NOV5

gnl|Pfam|pfam00069, pkinase, Protein kinase domain.
CD-Length = 256 residues, 97.3% aligned
Score = 200 bits (508), Expect = 2e−52

| | | | | |
|---|---|---|---|---|
| NOV 5: | 93 | FQILRAIGKGSFGKVVCIVQKRDTEKMYAMKYMNKQQCIERDEVRNVFRELEILQEIEHV | 152 | (SEQ ID NO:161) |
| | | +++ +\| \|+\|\|\|\| + +\|\| ++ \|+\| + \|+ \| + + \|\|++\|\|+ + \| | | |
| Sbjct: | 1 | YELGEKLGSGAFGKVY-KGKHKDTGEIVAIKILKKRSLSE--KKKRFLREIQILRRLSHP | 57 | (SEQ ID NO:162) |
| NOV 5: | 153 | FLVNLWYSFQDEEDMFMVVDLLLGGDLRYHLQQN-VQFSEDTVRLYICEMALALDYLRGQ | 211 | |
| | | +\| \| \|++++ +++\|++ + \|\|\|\| +\|++\| + \|\| + ++ \|+\|\| + | | |
| Sbjct: | 58 | NIVRLLGVFEEDDHLYLVMEYMEGGDLFDYLRRNGLLLSEKEAKKIALQILRGLEYLHSR | 117 | |
| NOV 5: | 212 | HIIHRDVKPDNILLDERGHAHLTDFNIATIIK--DGERATALAGTKPYMAPEIFHSFVNG | 269 | |
| | | \|+\|\|\|+\|\|+\|\|\|\|\|\| \| + \|\| +\| ++ \|+ \| \|\| \|\|\|\|\|+ | | |
| Sbjct: | 118 | GIVHRDLKPENILLDENGTVKIADFGLARKLESSSYEKLTTFVGTPEYMAPEVL-----E | 172 | |
| NOV 5: | 270 | GTGYSFEVDWWSVGVMAYELLRGWRPY-DIHSSNAVESLVQLFSTVSVQYVPTWSKEMVG | 328 | |
| | | \| \|\|\| +\|\| \|\|+\|\|+ \|\|\|\| \| \|+ \| + + + + + \| \|+\|+ | | |
| Sbjct: | 173 | GRGYSSKVDVWSLGVILYELLTGKLPFPGIDPLEELFRIKE-RPRLRLPLPPNCSEELKD | 231 | |
| NOV 5: | 329 | LLRKVLLTVNPEHRLSSLQ 347 | | |
| | | \|++\| \| +\|\| \| ++ + | | |
| Sbjct: | 232 | LIKK-CLNKDPEKRPTAKE 249 | | |

TABLE 5G

Domain Analysis of NOV5

gnl|Smart|smart00219, TyrKc, Tyrosine kinase, catalytic domain;
Phosphotransferases. Tyrosine-specific kinase subfamily.
CD-Length = 258 residues, 83.7% aligned
Score = 100 bits (250), Expect = 1e−22

| | | | | |
|---|---|---|---|---|
| NOV 5: | 95 | ILRAIGKGSFGKVV--CIVQKRDTEKMYAMKYMNKQQCIERDEVRNVFRELEILQEIEHV | 152 | (SEQ ID NO:163) |
| | | + + +\|+\|+\|\|+\| + \| \| \|+\| + + + ++ \|\| ++++++\| | | |
| Sbjct: | 3 | LGKKLGEGAFGEVYKGTLKGKGGVEVEVAVKTLKEDASEQ--QIEEFLREARLMRKLDHP | 60 | (SEQ ID NO:164) |
| NOV 5: | 153 | FLVNLWYSFQDEEDMFMVVDLLLGGDLRYHLQQN--VQFSEDTVRLYICEMALALDYLRG | 210 | |
| | | +\| \| +\|\| + +\|++ + \|\|\|\| +\|++\| + \| + + ++\| ++\|\| | | |
| Sbjct: | 61 | NIVKLLGVCTEEEPLMIVMEYMEGGDLLDYLRKNRPKELSLSDLLSFALQIARGMEYLES | 120 | |
| NOV 5: | 211 | QHIIHRDVKPDNILLDERGHAHLTDFNIATIIKDGE-RATALAGTKP--YMAPEIFHSFV | 267 | |
| | | ++ +\|\|\|+ \| \|+ \| + \|\| +\| + \| + + \| +\|\|\|\| | | |
| Sbjct: | 121 | KNFVHRDLAARNCLVGENKTVKIADFGLARDLYDDDYYRKKKSPRLPIRWMAPESLKDGK | 180 | |
| NOV 5: | 268 | NGGTGYSFEVDWWSVGVMAYELL-RGWRPYDIHSSNAVESLVQ 309 | | |
| | | ++ + \| \|\| \|\|+ +\|+ \| \|\| \|+ \| ++ | | |
| Sbjct: | 181 | -----FTSKSDVWSFGVLLWEIFTLGESPYPGMSNEEVLEYLK 218 | | |

Eukaryotic protein kinases are enzymes that belong to a very extensive family of proteins which share a conserved catalytic core common with both serine/threonine and tyrosine protein kinases. Protein phosphorylation is a fundamental process for the regulation of cellular functions. The coordinated action of both protein kinases and phosphatases controls the levels of phosphorylation and, hence, the activity of specific target proteins. One of the predominant roles of protein phosphorylation is in signal transduction, where extracellular signals are amplified and propagated by a cascade of protein phosphorylation and dephosphorylation events. Two of the best characterized signal transduction pathways involve the cAMP-dependent protein kinase and protein kinase C (PKC). Each pathway uses a different second-messenger molecule to activate the protein kinase, which, in turn, phosphorylates specific target molecules. Extensive comparisons of kinase sequences defined a common catalytic domain, ranging from 250 to 300 amino acids. This domain contains key amino acids conserved between kinases and are thought to play an essential role in catalysis. In the N-terminal extremity of the catalytic domain there is a glycine-rich stretch of residues in the vicinity of a lysine residue, which has been shown to be involved in ATP binding. In the central part of the catalytic domain there is a conserved aspartic acid residue which is important for the catalytic activity of the enzyme.

Protein kinases and phosphatases regulate cell-cycle progression, transcription, translation, protein sorting and cell adhesion events that are critical to the inflammatory process. Two of the best-characterized immunosuppressants, cyclosporin and rapamycin, are also effective anti-inflammatory drugs. They act directly on protein phosphorylation and, as such, validate the concept that small-molecule modulators of phosphorylation cascades possess anti-inflammatory properties. Some examples of the role of serine/threonine protein kinases that are important in cell proliferation and disease include AKT, RAF1 and PIM1. Dudek et al. demonstrated that AKT is important for the survival of cerebellar neurons. Thus, the ‘orphan’ kinase moved center stage as a crucial regulator of life and death decisions emanating from the cell membrane. Holland et al. transferred, in a tissue-specific manner, genes encoding activated forms of Ras and Akt to astrocytes and neural progenitors in mice. These authors found that although neither activated Ras nor Akt alone was sufficient to induce glioblastoma multiforme (GBM) formation, the combination of activated Ras and Akt induced high-grade gliomas with the histologic features of human GBMs. These tumors appeared to arise after gene transfer to neural progenitors, but not after transfer to differentiated astrocytes. Increased activity of Ras is found in many human GBMs and Akt activity is increased in most of these tumors, implying that combined activation of these 2 pathways accurately models the biology of this disease. Another disease that involves yet another serine/threonine kinase is Peutz-Jeghers syndrome (PJS), an autosomal dominant disorder characterized by melanocytic macules of the lips, buccal mucosa, and digits, multiple gastrointestinal hamartomatous polyps, and an increased risk of various neoplasms. Jenne et al. identified and characterized the serine/threonine kinase STK11 and identified mutations in PJS patients. All 5 germline mutations were predicted to disrupt the function of the kinase domain. They concluded that germline mutations in STK11, probably in conjunction with acquired genetic defects of the second allele in somatic cells according to the Knudson model, caused the manifestations of PJS. These authors commented that PJS was the first cancer susceptibility syndrome identified that is due to inactivating mutations in a protein kinase and found mutations in the STK11 gene in 11 of 12 unrelated families with PJS. Ten of the 11 were truncating mutations. All were heterozygous in the germline. Su et al. found that of 53 PJS patients with cancer reported to that time, 6 (11%) were diagnosed with pancreatic adenocarcinoma. Su et al. presented evidence that the STK11 gene plays a role in the development of both sporadic and familial (PJS) pancreatic and biliary cancers. They found that in sporadic cancers, the STK11 gene was somatically mutated in 5% of pancreatic cancers and in at least 6% of biliary cancers examined. In the patient with pancreatic cancer associated with PJS, there was inheritance of a mutated copy of the STK11 gene and somatic loss of the remaining wild type allele. See: Hunter, (1991) Meth. Enzymol. 200: 3-37; Taylor et al., (1991) Science 253: 407-414; Bhagwat et al., (1999) October;4(10):472-479; Dudek et al., (1997) Science 275: 661-663; Holland et al., (2000) Nature Genet. 25: 55-57; Jenne et al., (1998) Nature Genet. 18: 38-43; and Su et al., (1996) J. Biol. Chem. 271: 14430-14437.

The novel human serine/threonine protein kinase of the invention contains a protein kinase domain. Therefore it is anticipated that this novel protein has a role in the regulation of essentially all cellular functions and could be a potentially important target for drugs. Such drugs may have important therapeutic applications, such as treating numerous inflammatory diseases.

The protein similarity information, expression pattern, cellular localization, and map location for the NOV4 and NOV5 proteins and nucleic acids disclosed herein suggest that these Ser/Thr Protein Kinase-like proteins may have important structural and/or physiological functions characteristic of the Protein Kinase family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: Systemic lupus erythematosus, Autoimmune disease, Asthma, Emphysema, Scleroderma, Cancer, Fertility disorders, Reproductive disorders, Tissue/Cell growth regulation disorders, Developmental disorders as well as other diseases, disorders and conditions.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example, the disclosed NOV4 and NOV5 proteins have multiple hydrophilic regions, each of which can be used as an immunogen. In one embodiment, a contemplated NOV4 epitope is from about amino acids 40 to 52. In another embodiment, a contemplated NOV4 epitope is from about amino acids 60 to 65. In other specific embodiments, contemplated NOV4 epitopes are from about amino acids 90 to 110, 120 to 135, 160 to 168, 210 to 212, 260 to 275 and 310 to 315. In one embodiment, a contemplated NOV5 epitope is from about amino acids 45 to 55. In another embodiment, a contemplated NOV5 epitope is from about amino acids 120 to 150. In other specific embodiments, contemplated NOV5 epitopes are from about amino acids 160 to 170, 215 to 225, 280 to 310, 350 to 375, 390 to 420 and 440 to 455.

NOV6

A disclosed NOV6 nucleic acid (designated as CuraGen Acc. No. CG56684-02), encodes a novel Glycodelin-like protein and includes the 581 nucleotide sequence (SEQ ID NO:19) shown in Table 6A. An open reading frame for the mature protein was identified beginning with an ATG codon at nucleotides 36-38 and ending with a TAG codon at nucleotides 549-551. Putative untranslated regions downstream from the termination codon and upstream from the initiation codon are underlined in Table 6A, and the start and stop codons are in bold letters.

TABLE 6A
NOV6 Nucleotide Sequence

(SEQ ID NO:19)

CACTCCAGAGCTCAGAGCCACCCACAGCCACAGCTATGCAGTGCCTCCTGCTCACCCTGAGCATGGCCCTGGTC
TGTGCCATCCAGGCCAGGGACATCCCCCAGACCAAGCAGGACGTGGAGCTCCCAAAGTTGGCAGGGACCTGGTA
CTCCATGGCCATGGTGGCCAGTGACTTCTCCCTCCTGGAGACCGTGGAGGCCCCTCTGAGGGTCAACATCACCT
CGCTGTGGCCCACCCCCGAGGGCAACCTGGAGATCATTCTGCACAGATGGGAACACCACAGATGCGTTGAGAGG
ACCGTCCTCGCCCAGAAGACTGAGGACCCGGCTGTGTTCATGGTCGACCGTAGCAGGAGCTACGTGTTCTTCTG
CATGGGGACCACCACACCCAGTGCTGACCACCACACGATGTGCCAGTACCTGGGGATGACAGCCAGGACCCTAG
AGGCAGACGACAAGGTCATGGAGGAATTCATCAGCTTTCTCAGGACCCTGCCCGTGCACATGTGGATCTTCCTG
GACGTTACCCAGGCGGAACAGTGCCGCGTCTAGATGAGCTCCTGCTCAGTCCTGCCTCCTGGG

The nucleic acid sequence of NOV6 maps to chromosome 9 has 293 of 346 bases (84%) identical to a gb:GENBANK-ID:HUMENDOA21acc:M61886.1 mRNA from Homo sapiens (Human pregnancy-associated endometrial alpha2-globulin mRNA, complete cds) (E=1.4e⁻⁶⁶).

A disclosed NOV6 polypeptide (SEQ ID NO:20) is 171 amino acid residues in length and is presented using the one-letter amino acid code in Table 6B. The SignalP, Psort and/or Hydropathy results predict that NOV6 has a signal peptide and is likely to be localized outside of the cell with a certainty of 0.5899. In alternative embodiments, a NOV6 polypeptide is located to the microbody (peroxisome) with a certainty of 0.1391, the endoplasmic reticulum (membrane) with a certainty of 0.1000, or the endoplasmic reticulum (lumen) with a certainty of 0.1000. The SignalP predicts a likely cleavage site for a NOV6 peptide between amino acid positions 18 and 19, ie. at the sequence IQA-RD.

TABLE 6B
Encoded NOV6 Protein Sequence

(SEQ ID NO:20)

MQCLLLTLSMALVCAIQARDIPQTKQDVELPKLAGTWYSMAMVASDFSLLETVEAPLRVNITSLWPTPEGNLEIIL
HRWEHHRCVERTVLAQKTEDPAVFMVDRSRSYVFFCMGTTTPSADHHTMCQYLGMTARTLEADDKVMEEFISFLRT
LPVHMWIFLDVTQAEQCRV

The NOV6 amino acid sequence was found to have 110 of 186 amino acid residues (59%) identical to, and 132 of 186 amino acid residues (70%) similar to, the 186 amino acid residue ptnr:SPTREMBL-ACC:O77511 protein from Papio cynocephalus (Yellow baboon) (BETA-LACTOGLOBULIN 1) (E=3.2e⁻⁴⁷).

NOV6 is expressed in at least the following tissues because of the expression pattern of (GENBANK-ID: gb:GENBANK-ID:HUMENDOA2|acc:M61886.1) a closely related Human pregnancy-associated endometrial alpha2-globulin mRNA, complete cds homolog in species Homo sapiens: endometrium, amnion, and in semen.

NOV6 has homology to the amino acid sequences shown in the BLASTP data listed in Table 6C.

TABLE 6C
BLAST results for NOV6

| Gene Index/ | | Length | Identity | Positives | |
|---|---|---|---|---|---|
| Identifier | Protein/Organism | (aa) | (%) | (%) | Expect |
| gi\|17468008\| | similar to | 187 | 131/180 | 131/180 | 2e−63 |
| ref\|XP_070794.1\| | hypothetical protein | | (72%) | (72%) | |
| (XM_070794) | (H. sapiens) [Homo | | | | |
| | sapiens] | | | | |
| gi\|3483096\|gb\| | beta-lactoglobulin I | 186 | 112/192 | 136/192 | 2e−49 |
| AAC33251.1\| | [Papio cynocephalus] | | (58%) | (70%) | |
| (AF021261) | | | | | |
| gi\|130701\|sp\| | Glycodelin precursor | 180 | 98/184 | 127/184 | 7e−44 |
| P09466\|PAEP_{—} | (GD) (Pregnancy- | | (53%) | (68%) | |
| HUMAN | associated | | | | |
| | endometrial alpha-2 | | | | |
| | globulin) (PEG) | | | | |
| | (PAEG) (Placental | | | | |
| | protein | | | | |
| | 14) (Progesterone- | | | | |
| | associated | | | | |
| | endometrial | | | | |
| | protein) (Progestagen- | | | | |
| | associated | | | | |
| | endometrial protein) | | | | |
| gi\|4884164\|emb\| | hypothetical protein | 188 | 98/184 | 127/184 | 1e−43 |
| CAB43305.1\| | [Homo sapiens] | | (53%) | (68%) | |
| (AL050169) | | | | | |
| gi\|125905\|sp\| | BETA-LACTOGLOBULIN | 163 | 85/164 | 112/164 | 2e−37 |
| P21664\|LACA_{—} | II | | (51%) | (67%) | |
| FELCA | | | | | |

The homology of these sequences is shown graphically in the ClustalW analysis shown in Table 6D.

[image]

Table 6E list the domain description from DOMAIN analysis results against NOV6. This indicates that the NOV5 sequence has properties similar to those of other proteins known to contain these domains.

TABLE 6E
Domain Analysis of NOV6

gnl|Pfam|pfam00061, lipocalin, Lipocalin/cytosolic fatty-acid binding
protein family. Lipocalins are transporters for small hydrophobic molecules,
such as lipids, steroid hormones, bilins, and retinoids. Alignment subsumes
both the lipocalin and fatty acid binding protein signatures from PROSITE.
This is supported on structural and functional grounds. Structure is an eight-
stranded beta barrel.
CD-Length = 145 residues, 100.0% aligned
Score = 87.8 bits (216), Expect = 5e-19

| | | | | |
|---|---|---|---|---|
| NOV 6: | 32 | KLAGTWYSMAMVASDFSLLETVEAPLRVNITSLWPTPEGNLEIILHRWEHHRCVERTVLA | 91 | (SEQ ID NO:170) |
| | | \| \|\| \|\| +\| \| \| \| + \| + \| \|\|\|\|\|\|+ ++ \| \| | | |
| Sbjct: | 1 | KFAGKWYLVASANFDPELKEEL-GVLEATRKEITPLKEGNLEIVFDGDKNGICEETFGKL | 59 | (SEQ ID NO:171) |
| NOV 6: | 92 | QKTEDPAVFMVDRSR------------SYVFFCMGTTTPSADHHTMCQYLGMTARTLEAD | 139 | |
| | | +\|\|+ \| + +\|+ \|+ + + \|\| \| | | |
| Sbjct: | 60 | EKTKKLGVEFDYYTGDNRFVVLDTDYDNYLLVCVQ-KGDGNETSRTAELY---GRTPELS | 115 | |
| NOV 6: | 140 | DKVMEEFISFLRTLPVHMWIFLDVTQAEQC | 169 | |
| | | + +\| \| + + \| + + \| \|+\| | | |
| Sbjct: | 116 | PEALELFETATKELGIPEDNVVCTRQTERC | 145 | |

The protein of the invention exhibits sequence similarity to glycodelin and members of the lipocalin family, whose properties are described below. Based on the similarity to these proteins, the invention is likely to possess similar expression pattern, properties, or physiological function or role in disease. Placental protein-14 is synthesized by the human secretory endometrium and decidua. It is abundantly secreted by the human endometrium under the influence of progesterone. Julkunen et al. (1988) isolated cDNA clones corresponding to PP14 is encoded by a 1-kilobase mRNA that is expressed in secretory endometrium and decidua but not in postmenopausal endometrium, placenta, liver, kidney, and adrenals. The 162-residue-long sequence of PP14 is highly homologous to beta-lactoglobulin, the main component of equine, bovine, and ovine milk whey. Morris et al. (1996) reported that PP14, which they called glycodelin (Gd), exists as 2 gender-specific forms that differ in their glycosylation patterns. GdA, found in amniotic fluid, inhibits sperm-zona pellucida binding in an established sperm-egg binding system; GdS, found in seminal plasma, does not. Both forms suppress responses by a variety of immune effector cell types.

Lipocalins are a group of extracellular proteins, first described by Pervaiz and Brew (1987), that are able to bind lipophiles by enclosure within their structures, minimizing solvent contact. Based on the known 3-dimensional structure of 5 members of the lipocalin family, ie., retinol binding protein, beta-lactoglobulin, bilin binding protein, mouse major urinary protein, and rat urinary alpha-2-globulin, the general architecture appears to be highly appropriate for binding a variety of hydrophobic ligands. On the basis of highly conserved amino acid sequences and of a size around 18 to 20 kD, about 20 proteins have been designated as lipocalins. In tear fluid, a group of 6 proteins with molecular weights ranging from 15 to 20 kD and various isoelectric points are abundant. The N-terminal sequences of these proteins led Lassagne and Gachon (1993) to hypothesize that they are isoforms and belong to the lipocalin family. Tear prealbumin cDNA (Redl et al. (1992)) from lacrimal gland encodes a 176-amino acid protein that shares 58% identity to the von Ebner gland protein of the rat and significant homology with other lipocalins including beta lactoglobulin. From genetic and biochemical data, tear prealbumin is considered a member of the lipophilic-ligand carrier protein superfamily. Though tear prealbumin was originally described as a tear-specific protein, Redl et al. (1992) showed that tear prealbumin-specific antiserum reacted with human saliva, sweat, and nasal mucus proteins.

Von Ebner glands (VEG) are small lingual salivary glands. Their ducts open into trenches of circumvallate and foliate papillae, and their secretions influence the milieu where the interaction between taste receptor cells and sapid molecules (‘sapid’ means ‘possessing taste’) takes place. The major secretion of human VEG is a protein with a molecular mass of 18 kD. This VEG protein is identical to lipocalin-1. Blaker et al. (1993) isolated a cDNA clone from a human VEG library and showed that it contained an insert of 735 bp, including an open reading frame that encodes the human VEG protein of 176 amino acids. The VEG proteins are members of the lipocalin protein superfamily; together with odorant-binding protein, they constitute a new subfamily. Sequence similarity to proteins such as retinol binding protein and odorant binding protein suggests a possible function for the human VEG protein in taste perception.

Other members of the lipocalin family include: orosomucoid, alpha-1-microglobulin, progestagen-associated endometrial protein, the gamma chain of C8, and prostaglandin D2 synthase.

Using Northern blotting and immunohistology, Holzfeind et al. (1996) found that LCN1 is expressed in the human prostate. Cloning and sequencing showed that the transcript is identical to that found in tears. This finding suggested to Holzfeind et al. (1996) that the lipocalin-1 protein is not specific to tears and saliva, as was previously believed, but is multifunctional.

Van't Hof et al. (1997) showed that LCN1 inhibits the cysteine-protease papain in vitro, similar to cystatins (see 123857). They suggested that LCN1 plays a role in the nonimmunologic defense and in the control of inflammatory processes in oral and ocular tissues.

Redl et al. (1998) found enhanced LCN1 secretion in the airways of patients with cystic fibrosis (CF; 219700). Northern blot analysis of RNA from normal trachea and RNA isolated from tracheal biopsies of patients with CF indicated that the enhanced secretion was due to an upregulated expression of the LCN1 gene. Thus, the investigations presented the first clear evidence that LCN1 is induced in infection or inflammation and supported the idea that this lipocalin functions as a physiologic protection factor of epithelia in vivo.

The protein similarity information, expression pattern, and map location for the Glycodelin-like protein and nucleic acid disclosed herein suggest that this Glycodelin may have important structural and/or physiological functions characteristic of the Lipocalin family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The NOV6 nucleic acids and proteins of the invention are useful in potential diagnostic and therapeutic applications implicated in various diseases and disorders described below and/or other pathologies. For example, the compositions of the present invention will have efficacy for treatment of patients suffering from: infertility, endometriosis, other reproductive health disorders, lachrymal disorders, cancer, inflammation, autoimmune diseases and other diseases, disorders and conditions of the like.

The novel NOV6 nucleic acid encoding the Glycodelin-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV6 protein has multiple hydrophilic regions, each of which can be used as an immunogen. In one embodiment, a contemplated NOV6 epitope is from about amino acids 25 to 35. In another embodiment, a contemplated NOV6 epitope is from about amino acids 70 to 75. In other specific embodiments, contemplated NOV6 epitopes are from about amino acids 85 to 90, 92 to 98, 110 to 115, 130 to 139 and 148 to 150.

NOV7

A disclosed NOV7 nucleic acid (alternatively referred to herein as CG56977-01) encodes a novel Neuropathy Target Esterase/Swiss Cheese Protein-like protein and includes the 4718 nucleotide sequence (SEQ ID NO:21) shown in Table 7A. An open reading frame for the mature protein was identified beginning with an ATG codon at nucleotides 1-3 and ending with a ATC codon at nucleotides 4258-4260. Putative untranslated regions are underlined in Table 7A, and the start and stop codons are in bold letters.

TABLE 7A
NOV7 Nucleotide Sequence

(SEQ ID NO:21)

ATGGAGGAAGAGAAAGATGACAGCCCACAGCTGACGGGGATTGCAGTTGGAGCCCTCCTGGCCCTGGCCTTGGTTGG
TGTCCTCATCCTTTTCATGTTCAGAAGGCTTAGACAATTTCGACAAGCACAGCCCACTCCTCAGTACCGGTTCCGGA
AGAGAGACAAAGTGATGTTTTACGGCCGGAAGATCATGAGGAAGGTGACCACACTCCCCAACACCCTTGTGGAGAAC
ACTGCCCTGCCCCGGCAGCGGGCCACGAAGAGGACCAAGGTGCTGTCTTTGGCCAAGAGGATTCTGCGTTTCAAGAA
GGAATACCCGGCCCTGCAGCCCAAGGAGCCCCCGCCCTCCCTGCTGGAGGCCGACCTCACGGAGTTTGACGTGAAGA
ATTCTCACCTGCCATCGGAAGTTCTGTACATGCTGAAAAACGTTCGGGTCCTGGGCCACTTTGACAAGCCGCTGTTC
CTGGAGCTTTGCAAACACATCGTCTTTGTGCAGCTGCAGGAAGGGGAGCACGTCTTCCAGCCCAGGGAGCCGGACCC
CAGCATCTGTGTGGTGCAGGACGGGCGGGTGGAGGTCTGCATCCAGGACACTGACGGCACCGAGGTGGTGGTGAAAG
AGGTTCTGGCGGGAGACAGCGTCCACAGCCTGCTCAGCATCCTGGACATCATCACCGGCCATGCTGCACCTTACAAA
ACGGTCTCCGTCCGCGCGGCCATCCCGTCCACCATCCTCCGGCTTCCAGCTGCGGCTTTTCATGGAGTTTTTGAGAA
ATATCCGGAAACTCTGGTGAGGGTGGTGCAGTTGCAGATCATCATGGTGCGGCTGCAGAGGGTGACCTTTCTGGCTC
TGCACAACTACCTCGGCCTGACCACAGAGCTCTTCAACGCTGAGAGCCAGGCCATCCCTCTCGTGTCTGTAGCCAGT
GTGGCTGCCGGGAAGGCCAAGAAGCAGGTGTTCTATGGCGAAGAAGAGCGGCTTAAAAAGCCACCGCGGCTCCATGA
GTCCTGTGACTCAGCAGATCACGGGGGCGGCCGCCCGGCAGCTGCTGGGCCCCTGCTGAAGAGGAGCCACTCCGTCC
CCGCGCCTTCCATTCGGAAACAGATCTTGGAGGAGCTGGAGAAGCCCGGGGCAGGTGACCCTGACCCTTCGGCCCCA
CAAGGGGGCCCAGGCAGTGCCACTTCTGATCTGGGGATGGCATGTGACCGTGCCAGGGTCTTCCTGCACTCGGACGA
GCACCCCGGGAGCTCCGTGGCCAGCAAGTCCAGGAAAAGCGTGATGGTTGCAGAGATACCCTCCACGGTCTCCCAGC
ACTCAGAGAGTCACACGGATGAGACCCTGGCCAGCAGGAAGTCGGATGCCATCTTCAGAGCTGCCAAGAAGGACCTG
CTCACCCTGATGAAGCTGGAAGACTCATCTCTGTTGGATGGCCGGGTGGCGCTTCTGCACGTTCCTGCATGCACGGT
GGTGTCAATGCAGGGAGACCAAGACGCCAGCATCCTGTTCGTTGTCTTGGGGCTGCTGCACGTGTACCAGCGGAAGA
TCTGCAGCCAGGAGGACACCTGCTTGTTCTCACGCGCACCCGGGGACTCATCTCTGTTGGATGGCCGGGTGGCGCTT
CTGCACGTTCCTGCAGGCACGGTGGTGTCAAGGCAGGGAGACCAGGACGCCAGCATCCTGTTCGTGGTCTCGGGGCT
GCTGCACGTGTACCAGCGGAAGATCGGCAGCCAGGAGGACACCTGCTTGTTCCTCACGCGCCCCGGGGAGATGGTGG
GCCAGCTGGCCGTGCTCACCGGGGAGCCTCTCATCTTCACCGTCAAGGCCAACAGGGACTGCAGCTTCCTGTCCATC
TCCAAGGCCCACTTCTATGAAATCATGCGGAAGCAGCCGACCGTCGTCCTGGGTGTGGCGCACACTGTGGTGAAGAG
GATGTCGTCCTTCGTGCGGCAAATCGACTTTGCCCTGGACTGGGTGGAGGTGGAGGCCGGGCGAGCAATATACAGGC
AGGGGGACAAGTCCGACTGCACGTACATCATGCTCAGCGGCCGGCTGCGCTCTGTGATCCGGAAGGATGATGGGAAG
AAGCGCCTGGCCGGGGAGTACGGCCGAGGAGACCTCGTCGGCGTGGTGGAGACACTGACCCACCAGGCCCGGGCGAC
CACGGTGCATGCCGTTCGGGACTCAGAATTGGCCAAGCTGCCGGCAGGAGCCCTCACGTGCATCAAGCGCAGGTACC
CACAGGTGGTGACTCGGCTGATTCATCTCTTCGGTGAGAAGATCCTGGGCAGCCTCCAGCAGGGACCTGTGACAGGC
CACCAGCTTGGGCTCCCCACGGAGGGCAGCAAGTGGGACTTGGGGAACCCGGCTGTCAACCTGTCCACGGTGGCAGT
GATGCCCGTGTCAGAGGAAGTGCCCCTCACCGCCTTCGCCCTGGAGCTGGAGCATGCCCTCAGCGCCATCGGCCCGC
CCCTGCTGCTGACTAGTGACAACATAAAACGGCGCCTTGGCTCCGCTGCCCTGGACAGTGTTCACGAGTACCGGCTG
TCCAGCTGGCTGGGGCAGCAGGAGGACACCCACAGGATCGTGCTCTACCAGGTAGATGGCACGCTCACACCCTGGAC
CCAGCGCTGCGTGCGCCAGGCCGACTGCATCCTCATCGTGGGCCTGGGTGACCAGGAGCCCACAGTGGGCGAGCTGG
AGCGGATGCTGGAGAGCACAGCTGTGCGTGCCCAGAAGCAGCTGATCCTGCTGCACAGGGAGGAGCGCCCGGCGCCA
GCGCGCACCGTGGAGTGGCTCAACATGCGGAGCTGGTGCTCCGGCCACCTGCACCTCTGCTGCCCGCGCCGCGTCTT
CTCCAGGAGGAGCCTGCCCAAGCTGGTGGAGATGTACAAGCATGTCTTCCAGCGGCCCCCGCACCGACACTCAGACT
TCTCCCGCCTGGCGAGGGTGCTGACGGGCAACGCCATTGCCCTGGTGCTTGCGGGAGGGGGAGCAAGCATGACGTCC
TTGATGAAGGCCGCGCTGGACCTCACCTACCCCATCACGTCCATGTTCTCCGGAGCCGGCTTCAACAGCAGCATCTT
CAGCGTCTTCAAGGACCAGCAGATCGAGGACCTGTGGATTCCTTATTTCGCCATCACCACCGACATCACAGCCTCGG
CCATGCGGGTCCACACCGACGGCTCCCTGTGGTGGTACGTGCGTGCCAGCATGTCCCTGTCCGGTTACATGCCCCCT
CTCTGTGACCCGAAGGACGGACACCTGCTGATGGACGGGGGCTACATCAACAACCTCCCAGCTGCCTCCGCTCCAAG
AAGCCTGGGCTGGAACACGTTTTCCTTAGAGTATGCCAAGGGAAAATGTCAGGCTGGCATCAGAGCTCCGAGAACAT
GCACACGCGTGTACATGCACACGCAGGCACCGGCAGCATGTGCTCCAGCATATGGCCCTGTTTGTCAGCTCAGCAGC
ATGCAGAACAAAGGCCAAGTCGAGGAACTGGGAGCAATTAAGCCCCATCTGTGCCCACAGTCAGAAACTAACAGCCT
GCAGGGGGTAACCAGGGCTGGCTTCTCCCTAGCGGATGTGGCCCGGTCCATGGGGGCAAAAGTGGTGATCGCCATTG
ACGTGGGCAGCCGAGATGAGACGGACCTCACCAACTATGGGGATGCGCTGTCTGGGTGGTGGCTGCTGTGGAAACGC
TGGAACCCCTTGGCCACGAAAGTCAAGGTGTTGAACATGGCAGAGATTCAGACGCGCCTGGCCTACGTGTGTTGCGT
GCGGCAGCTGGAGGTGGTGAAGAGCAGTGACTACTGCGAGTACCTGCGCCCCCCCATCGACAGCTACAGCACCCTGG
ACTTCGGCAAGTTCAACGAGATCTGCGAAGTGGGCTACCAGCACGGGCGCACGGTGTTTGACATCTGGGGCCGCAGC
GGCGTGCTGGAGAAGATGCTCCGCGACCAGCAGGGGCCGAGCAAGAAGCCCGCGAGTGCGGTCCTCACCTGTCCCAA
CGCCTCCTTCACGGACCTTGCCGAAATTGTGTCTCGCATTGAGCCCGCCAAGCCCGCCATGGTGGATGACGAATCTG
ACTACCAGACGGAGTACGAGGAGGAGCTGCTGGACGTCCCCAGGGATGCATACGCAGACTTCCAGAGCACCTCAGCC
CAGCAGGGCTCAGACTTGGAGGACGAGTCCTCACTGCGGCATCGACACCCCAGTCTGGCTTTCCCAAAACTGTCTGA
GGGCTCCTCTGACCAGGACGGGTAGAGGCCTCTGCTAAAGAGCCCGGATGCAGCGTCTTCCGTGGGACTGTCCCCAA
GGCTGAGGCTCCTGCCAAGTCCTAGGGGCCTCTGTACCTGCCCTGCTGGAAGCCCTGACTTCCCCGGGGCCCCAGGC
TGTGTTAGGGTTCTCTGGGCCTCTTCTTTGTACCAGCAGCCCTGCATACAGGGCCCTGTGAGCCCCCCTGCAGTCCT
GTGAGGCCCCTGAAGCTCTGTGAGGCCCCTGAAGCTCTGTGAACCCCCTGCAGCCCTGTGAGGCCCCCCGAAGCCCT
GTGAGGCCCCCCGAAGCCCTGTGAACCACCTGCTGCCCTGTGAGGCCCCCAAAGCCCTGTGAACTGCCTGCTGTCCT
GTGAACTGCCTGCTGCCCTGTGAGGTGTGGGAGCCCTGATGCTGCCGTGTGATGTTTCAATAAAGGTGGATCTCACT
GTTGAAAAAAAAAAAAAAAAA

The nucleic acid sequence of NOV7 maps to chromosome 9 and invention has 1104 of 1504 bases (73%) identical to a gb:GENBANK-ID:HSAJ4832|acc:AJ004832.1 mRNA from Homo sapiens (Homo sapiens mRNA for neuropathy target esterase) (E 0.0).

A disclosed NOV7 polypeptide (SEQ ID NO:22) is 1419 amino acid residues in length and is presented using the one-letter amino acid code in Table 7B. The SignalP, Psort and/or Hydropathy results predict that NOV7 has a signal peptide and is likely to be localized to the endoplasmic reticulum (membrane) with a certainty of 0.8200. In alternative embodiments, a NOV7 polypeptide is located to the nucleus with a certainty of 0.2400, the plasma membrane with a certainty of 0.1900, or the endoplasmic reticulum (lumen) with a certainty of 0.1000. The SignalP predicts a likely cleavage site for a NOV7 peptide between amino acid positions 38 and 39, i.e. at the sequence LRQ-FR.

TABLE 7B
Encoded NOV7 Protein Sequence

(SEQ ID NO:22)

MEEEKDDSTQLTGIAVGALLALALVGVLILFMFRRLRQFRQAQPTPQYRFRKRDKVMFYGRKIMRKVTTLPNTLV
ENTALPRQRARKRTKVLSLAKRILRFKKEYPALQPKEPPPSLLEADLTEFDVKNSHLPSEVLYMLKNVRVLGHFE
KPLFLELCKHIVFVQLQEGEHVFQPREPDPSICVVQDGRLEVCIQDTDGTEVVVKEVLAGDSVHSLLSILDIITG
HAAPYKTVSVRAAIPSTILRLPAAAFHGVFEKYPETLVRVVQLQIIMVRLQRVTFLALHNYLGLTTELFNAESQA
IPLVSVASVAAGKAKKQVFYGEEERLKKPPRLHESCDSADHGGGRPAAAGPLLKRSHSVPAPSIRKQILEELEKP
GAGDPDPSAPQGGPGSATSDLGMACDRARVFLHSDEHPGSSVASKSRKSVMVAEIPSTVSQHSESHTDETLASRK
SDAIFRAAKKDLLTLMKLEDSSLLDGRVALLHVPACTVVSMQGDQDASILFVVLGLLHVYQRKICSQEDTCLFSR
APGDSSLLDGRVALLHVPAGTVVSRQGDQDASILFVVSGLLHVYQRKIGSQEDTCLFLTRPGEMVGQLAVLTGEP
LIFTVKANRDCSFLSISKAHFYEIMRKQPTVVLGVAHTVVKRMSSFVRQIDFALDQVEVEAGRAIYRQGDKSDCT
YIMLSGRLRSVIRKDDGKKRLAGEYGRGDLVGVVETLTHQARATTVHAVRDSELAKLPAGALTCIKRRYPQVVTR
LIHLLGEKILGSLQQGPVTGHQLGLPTEGSKWDLGNPAVNLSTVAVMPVSEEVPLTAFALELEHALSAIGPPLLL
TSDNIKRRLGSAALDSVHEYRLSSWLGQQEDTHRIVLYQVDGTLTPWTQRCVRQADCILIVGLGDQEPTVGESER
MLESTAVRAQKQLILLHREEGPAPARTVEWLNMRSWCSGHLHLCCPRRVFSRRSLPKLVEMYKHVFQRPPDRHSD
FSRLARVLTGNAIALVLGGGGASMTSLMKAADLTYPITSMFSGAGFNSSIFSVFKDQQIEDLLWIPYFAITTDIT
ASAMRVHTDGSLWWYVRASMSLSGYMPPLCDPKDGHLLMDGGYINNLPAASAPRSLGWNTFSLEYAKGKCQAGIR
APRTCTRVYMHTQAPAACAPAYGPVCQLSSMQNKGQVEELGAIKPHLCPQSETNSLQGVTRAGFSLADVARSMGA
KVVIAIDVGSRDETDLTNYGDALSGWWLLWWRWNPLATKVKVLNMAEIQTRLAYVCCVRQLEVVKSSDYCEYLRP
PIDSYSTLDFGKFNEICEVGYQNGRTVFDIWGRSGVLEKMLRDQQGPSKKPASAVLTCPNASFTDLAEIVSRIEP
AKPAMVDDESDYQTEYEEELLDVPRDAYADFQSTSAQQGSDLEDESSLRHRHPSLAFPKLSEGSSDQDG

The NOV7 amino acid sequence was found to have 349 of 507 amino acid residues (68%) identical to, and 407 of 507 amino acid residues (80%) similar to, the 1327 amino acid residue ptnr:SPREMBL-ACC:Q9R114 protein from Mus musculus (Mouse) (NEUROPATHY TARGET ESTERASE HOMOLOG) (E=0.0).

NOV7 is expressed in at least the following tissues: blood, tonsil, lung tumor, and prostate (normal). Expression information was derived from the tissue sources of the sequences that were included in the derivation of the sequence of NOV7. The sequence is predicted to be expressed in the following tissues because of the expression pattern of (GENBANK-ID: gb:GENBANK-ID:HSAJ4832|acc:AJ004832.1) a closely related Homo sapiens mRNA for neuropathy target esterase homolog in species Homo sapiens: bone, brain, breast, germ cell, heart, kidney, lung, pancreas, pooled, prostate, testis, tonsil, uterus, whole embryo, amnion-normal, brain, breast, colon, head, neck, kidney, lung, placenta, prostate-normal, skin, and uterus.

Possible small nucleotide polymorphisms (SNPs) found for NOV7 are listed in Table 7C.

TABLE 7C
SNPs

| | Nucleotide | | Amino Acid | |
|---|---|---|---|---|
| Variant | Position | Base Change | Position | Base Change |
| 13375546 | 707 | G > A | 236 | Arg > His |
| 13376992 | 3984 | C > G | NA | NA |

NOV7 also has homology to the amino acid sequences shown in the BLASTP data listed in Table 7D.

TABLE 7D
BLAST results for NOV7

| Gene Index/ | Protein/ | Length | Identity | Positives | |
|---|---|---|---|---|---|
| Identifier | Organism | (aa) | (%) | (%) | Expect |
| gi\|7657401\|ref\|NP_{—} | neuropathy | 1327 | 650/1174 | 779/1174 | 0.0 |
| 056616.1\| | target | | (55%) | (65%) | |
| (NM_015801) | esterase; Swiss | | | | |
| | cheese [Mus | | | | |
| | musculus] | | | | |
| gi\|16550716\|dbj\| | unnamed protein | 702 | 420/483 | 421/483 | 0.0 |
| BAB71033.1\|(AK055880) | product [Homo | | (86%) | (86%) | |
| | sapiens] | | | | |
| gi\|17530839\|ref\|NP_{—} | swiss cheese; | 1425 | 447/1112 | 624/1112 | 0.0 |
| 511075.1\|(NM_078520) | olfactory E | | (40%) | (55%) | |
| | [Drosophila | | | | |
| | melanogaster] | | | | |
| gi\|7290863\|gb\| | sws gene | 1389 | 446/1111 | 623/1111 | 0.0 |
| AAF46305.1\| | product | | (40%) | (55%) | |
| (AE003442) | [Drosophila | | | | |
| | melanogaster] | | | | |
| gi\|5729951\|ref\|NP_{—} | neuropathy | 1327 | 272/548 | 351/548 | e−122 |
| 006693.1 | target esterase | | (49%) | (63%) | |
| (NM_006702) | [Homo sapiens] | | | | |

The homology of these sequences is shown graphically in the ClustalW analysis shown in Table 7E.

[image]

[image]

[image]

[image]

Tables 7F and 7G list the domain description from DOMAIN analysis results against NOV7. NOV7 shows similarity to an uncharacterized protein family and, at several positions, to a cyclic nucleotide binding domain/cyclic nucleotide monophosphate binding domain. This indicates that the NOV7 sequence has properties similar to those of other proteins known to contain these domains.

TABLE 7F
Domain Analysis of NOV7

gnl|Pfam|pfam01173, UPF0028, Uncharacterized protein family UPF0028.
CD-Length = 317 residues, 91.2% aligned
Score = 164 bits (416), Expect = 2e-41

| | | | | |
|---|---|---|---|---|
| NOV 7: | 970 | PDRHSDFSRLARVLTGNAIALVLGGGGA---SMTSLMKAALDLTYPITSMFSGAGFNSSI | 1026 | (SEQ ID NO:177) |
| | | \|\|\|\|\|\|\|\|+\|\|\|\|\|\| \|\|\|\|\|\|\|\| + +++\| ++ \|\| + \| \| + | | |
| Sbjct: | 4 | IAFQSDFSRLARILTGNAIGLVLGGGGARGAARIGVIQALKEVGIPI-DIVGGTSIGSLV | 62 | (SEQ ID NO:178) |
| NOV7: | 1027 | FSVFKDQQIEDLWIPYFAITTDITASAMRVHTDGSLWWYVRASNSLSGYMPPLCDPKDGH | 1086 | |
| | | +++ \| \| \| \|+ \| \|+ + \| \|+ \| | | |
| Sbjct: | 63 | GALY-------------ACDPDSVLV------DARAKWFFSGSSSIWDELMDLTWPRSG- | 102 | |
| NOV7: | 1087 | LLMDGGYINNLPAASAPRSLGWNTFSLEYAKGKCQAGIRAPRTCTRVYMHTQAPAACA-P | 1145 | |
| | | \|+ \| \| +\| + + \| + \|+ + | | |
| Sbjct: | 103 | -LLTGERPNRQVQEIFGETLIED-CWRSFFCVSTDLSTSRQRIHREGDLWLAIRASMSIA | 160 | |
| NOV7: | 1146 | AY-GPVCQLSSMQNKGQVEELGAIKPELCPQSETNSLQGVTRAGFSLADVARSMGAKVVI | 1204 | |
| | | \|\|\|\| + \| \|+\| \|\|\| \|++\|\| +\|\| | | |
| Sbjct: | 161 | GLLPPVCQNGHLLLDGGY---------------VNNLP---------ADVMRALGADIVI | 196 | |
| NOV7: | 1205 | AIDVGSRDETDLTNYGDALSGWWLLWKRWNPLATKVKVLNMAEIQTRLAYVCCVRQLEVV | 1264 | |
| | | \|+\|\|\|\| \| \|+\| \|\| +\|\|\| \|+\|+\|\|\|\|\| ++++\|\|\|+\|\|\| \|\|\|\|\| \|\|\| \|\| | | |
| Sbjct: | 197 | AVDVGSADLTNLDLYGFSLSGEWILFKRWNPFGARLRILNMSEIQRRLAYVPCVRALETA | 256 | |
| NOV 7: | 1265 | KSSDYCEYLRPPIDSYSTLDPGKFNEICEVGYQHGR | 1300 | |
| | | \|++ \|\| \|\|+ \|\|+++ \|\|\|\| \|\| \|\| ++\| + + | | |
| Sbjct: | 257 | KNTVYCRYLKRPIEAFDTLDFSKFPEIPQIGVLYFK | 292 | |

TABLE 7G
Domain Analysis of NOV7

gnl|Pfam|pfam00027, cNMP binding, Cyclic nucleotide-binding domain.
CD-Length = 94 residues, 100.0% aligned
Score = 78.6 bits (192), Expect = 2e-15

| | | | | |
|---|---|---|---|---|
| NOV 7: | 653 | ALDWVEVEAGPAIYRQGDKSDCTYIMLSGRLRSVIRKDDGKKRLAGEYGRGDLVGVVETL | 712 | (SEQ ID NO:179) |
| | | \|\|+ \|\| \| \|\|\|\| \| \|\|++\|\| + +\|\|++++ \| \| \|\|\| \| + \| | | |
| Sbj Ct: | 1 | ALEERSYPAGEVIIRQGDPGDSLYIVVSGSVEVYRLLEDGREQIVGTLGPGDLFGELALL | 60 | (SEQ ID NO:180) |
| NOV 7: | 713 | THQARATTVHAVRDSELAKLPAGALTCIKRRYPQ | 746 | |
| | | \|+ \| \|\| \|+ \| \|\| +\| + +\|\|+ | | |
| Sbjct: | 61 | TNPPRTATVRALTDCELLRLDREDFERLLEQYPE | 94 | |

gnl|Pfam|pfam00027, cNMP_binding, Cyclic nucleotide-binding domain.
CD-Length = 94 residues, 93.6% aligned
Score = 76.6 bits (187), Expect = 9e-15

| | | | | |
|---|---|---|---|---|
| NOV 7: | 541 | HVPAGTVVSRQGDQDASILFVVSGLLHVYQRKIGSQEDTCLFLTRPGEMVGQLAVLTGEP | 600 | (SEQ ID NO:181) |
| | | \|\|\| \|+ \|\|\|\| \|+ \|\|\|\| + \|\|+ +\| \| \|\|++ \|+\|\|+\|\| \| | | |
| Sbj Ct: | 6 | SYPAGEVIIRQGDPGDSLYIVVSGSVEVYRLLEDGREQIVGTL-GPGDLFGELALLTNPP | 64 | (SEQ ID NO:182) |
| NOV 7: | 601 | LIFTVKANRDCSFLSISKAHFYEIMRKQP | 629 | |
| | | \|\|+\| \|\| \| + + \| ++ + \| | | |
| Sbjct: | 65 | RTATVRALTDCELLRLDREDFERLLEQYP | 93 | |

gnl|Pfam|pfam00027, cNMP_binding, Cyclic nucleotide-binding domain.
CD-Length = 94 residues, 100.0% aligned
Score = 64.3 bits (155), Expect = 4e-11

| | | | | |
|---|---|---|---|---|
| NOV 7: | 160 | HIVFVQLQEGEHVFQPREPDPSICVVQDGRLEVCIQDTDGTEVVVKEVLAGDSVHSLLSI | 219 | (SEQ ID NO:183) |
| | | + \|\| + + +\| \|+ +\| \| +\|\| \|\| \| +\| + \|\| \| + | | |
| Sbjet: | 1 | ALEERSYPAGEVIIRQGDPGDSLYIVVSGSVEVYRLLEDGREQIVGTLGPGDLFGELALL | 60 | (SEQ ID NO:184) |
| NOV 7: | 220 | LDIITGHAAPYKTVSVRAAIPSTILRLPAAAFHGVFEKYPE | 260 | |
| | | + \| +\| +\|\|\| +\|\|\| \| + \|+\|\|\| | | |
| Sbjct: | 61 | TN-------PPRTATVRALTDCELLRLDREDFERLLEQYPE | 94 | |

gnl|Smart|smart00100, cNMP, Cyclic nucleotide-monophosphate binding domain;
Catabolite gene activator protein (CAP) is a prokaryotic homologue of eukaryotic
cNMP-binding domains, present in ion channels, and cNMP-dependent kinases.
CD-Length = 121 residues, 94.2% aligned
Score = 66.2 bits (160), Expect = 1e-11

| | | | | |
|---|---|---|---|---|
| NOV 7: | 645 | SFVRQIDFALDWVEVEAGRAIYRQGDKSDCTYIMLSGRLRSVIRKDDGKKRLAGEYGRGD | 704 | (SEQ ID NO:185) |
| | | +\|++ \|\|+ \| \|\| \| \|\|\|\| \| \|\|++\|\| + +\|\|++++ \| \| \|\| | | |
| Sbjct: | 8 | EELRELADALEPVRYPAGEVIIRQGDVGDSFYIIVSGEVEVYKTLEDGREQILGTLGPGD | 67 | (SEQ ID NO:186) |
| NOV 7: | 705 | LVGVVETLTHQARATTVHAVRDSELAKLPAGALTCIKRRYPQVVTRLIHLLGEKI | 759 | |
| | | \| + \|\|++ \|\| + \| \|\|\|\|\| + \|++ \|+ \|\| \| | | |
| Sbjct: | 68 | FFGELALLTNRRRARSA-AAVALELAKLLRIDFRDFLQLLPEIPQLLLELLLELA | 121 | |

gnl|Smart|smart00100, cNMP, Cyclic nucleotide-monophosphate binding domain;
Catabolite gene activator protein (CAP) is a prokaryotic homologue of eukaryotic
cNMP-binding domains, present in ion channels, and cNMP-dependent kinases.
CD-Length = 121 residues, 97.5% aligned
Score = 63.9 bits (154), Expect = 6e-11

| | | | | |
|---|---|---|---|---|
| NOV 7: | 145 | VLGHFEKPLFLELCKHIVFVQLQEGEHVFQPREPDPSICVVQDGRLEVCIQDTDGTEVVV | 204 | (SEQ ID NO:187) |
| | | + + \|\| + \|+ \|\| + + + \| ++ \| +\|\| \|\| \| ++ | | |
| Sbjct: | 1 | LFKALDAEELRELADALEPVRYPAGEVIIRQGDVGDSPYIIVSGEVEVYKTLEDGREQIL | 60 | (SEQ ID NO:188) |
| NOV 7: | 205 | KEVLAGDSVHSLLSILDIITGHAAPYKTVSVRAAIPSTILRLPAAAFHGVFEKYPETLVR | 264 | |
| | | + \|\| \| ++\| + + \| + +\|\|+ \| + + \|+ \|+ | | |
| Sbjct: | 61 | GTLGPGDFF----GELALLTNRRRAR-SAAAVALELAKLLRIDFRDFLQLLPEIPQLLLE | 115 | |
| NOV 7: | 265 | VVQ | 267 | |
| | | ++ | | |
| Sbjct: | 116 | LLL | 118 | |

gnl|Smart|smart00100, cNMP, Cyclic nucleotide-monophosphate binding domain;
Catabolite gene activator protein (CAP) is a prokaryotic homologue of eukaryotic
cNMP-binding domains, present in ion channels, and cNMP-dependent kinases.
CD-Length = 121 residues, 74.4% aligned
Score = 55.1 bits (131), Expect = 3e-08

| | | | | |
|---|---|---|---|---|
| NOV 7: | 541 | HVPAGTVVSRQGDQDASILFVVSGLLHVYQRKIGSQEDTCLFLTRPGEMVGQLAVLTGE- | 599 | (SEQ ID NO:189) |
| | | \|\|\| \|+ \|\|\|\| \| +\|\|\| + \|\|+ + + \| \|\|+ \|+\|\|+\|\| | | |
| Sbjct: | 21 | RYPAGEVIIRQGDVGDSFYIIVSGEVEVYKT-LEDGREQILGTLGPGDFFGELALLTNRR | 79 | (SEQ ID NO:190) |
| NOV 7: | 600 | -PLIFTVKANRDCSFLSISKAHFYEIMRKQP | 629 | |
| | | \| \| \| \| +++ + \| | | |
| Sbjct: | 80 | RARSAAAVALELAKLLRIDFRDFLQLLPEIP | 110 | |

Uncharacterized protein family UPF0028 (Interpro IPR001423): A number of prokaryotic and eukaryotic uncharacterized proteins belong to this family. These proteins are of variable size and share a glycine-rich domain of about 200 residues that is located at the C-terminus of the eukaryotic members of this family.

Cyclic nucleotide-binding domain (Interpro IPR000595): Proteins that bind cyclic nucleotides (cAMP or cGMP) share a structural domain of about 120 residues. The best studied of these proteins is the prokaryotic catabolite gene activator (also known as the cAMP receptor protein) (gene crp) where such a domain is known to be composed of three alpha-helices and a distinctive eight-stranded, antiparallel beta-barrel structure. There are six invariant amino acids in this domain, three of which are glycine residues that are thought to be essential for maintenance of the structural integrity of the beta-barrel. cAMP- and cGMP-dependent protein kinases (CAPK and cGPK) contain two tandem copies of the cyclic nucleotide-binding domain. The cAPK's are composed of two different subunits, a catalytic chain and a regulatory chain, which contains both copies of the domain. The cGPK's are single chain enzymes that include the two copies of the domain in their N-terminal section. Vertebrate cyclic nucleotide-gated ion-channels also contain this domain. Two such cations channels have been fully characterized, one is found in rod cells where it plays a role in visual signal transduction.

The novel protein of the invention is similar to Neuropathy Target Esterases and Swiss Cheese proteins and therefore is likely to share some of their properties which are described below. Covalent modification of Neuropathy Target Esterase (human NTE) by certain organophosphorus esters (OPs) leads, after a delay of several days, to a degeneration of long axons in the spinal cord and peripheral nerves (organophosphate-induced neuropathy). The active-site serine of NTE lies in the center of a predicted hydrophobic helix within a 200-amino-acid C-terminal domain with marked similarity to conceptual proteins in bacteria, yeast and nematodes; these proteins may comprise a novel family of potential serine hydrolases.

NTE shares 41% amino acid sequence identity with the Drosophila ‘Swiss Cheese’ (Sws) protein, which is involved in the regulation of interactions between neurons and glia in the developing fly brain. Swiss cheese (sws) mutant flies develop normally during larval life but show age-dependent neurodegeneration in the pupa and adult and have reduced life span. In late pupae, glial processes form abnormal, multilayered wrappings around neurons and axons. Degeneration first becomes evident in young flies as apoptosis in single scattered cells in the CNS, but later it becomes severe and widespread. In the adult, the number of glial wrappings increases with age. The sws gene is expressed in neurons in the brain cortex. It is suggested that the novel SWS protein plays a role in a signaling mechanism between neurons and glia that regulates glial wrapping during development of the adult brain.

The observation that the Swiss Cheese protein when mutated, leads to widespread cell death in Drosophila brain, suggests that genetically altered NTE, because of its homology to swiss cheese protein may be involved in human neurodegenerative disease. The murine sws/NTE gene is 96% identical to NTE. During development the Msws transcript is expressed in the embryonic respiratory system, different epithelial structures and strongly in the spinal ganglia. Postnatally, Msws mRNA is expressed in all brain areas, with an increasingly restrictive pattern. In adult mice expression is most prominent in Purkinje cells, granule cells and pyramidal neurons of the hippocampus and some large neurons in the medulla oblongata, nucleus dentatus and pons.

The novel Neuropathy Target Esterase/Swiss Cheese protein family member described in this invention is therefore anticipated to have similar biochemical and physiological roles as described above for family members.

The protein similarity information, expression pattern, cellular localization, and map location for the NOV7 protein and nucleic acid disclosed herein suggest that this Neuropathy target esterase/Swiss Cheese protein-like protein may have important structural and/or physiological functions characteristic of the Neuropathy target esterase/Swiss Cheese protein family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: cancer, trauma, regeneration (in vitro and in vivo), viral/bacterial/parasitic infections, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, aneurysm, hypertension, fibromuscular dysplasia, stroke, scleroderma, obesity, transplantation, myocardial infarction, embolism, cardiovascular disorders, bypass surgery, anemia, bleeding disorders, scleroderma, transplantation, adrenoleukodystrophy, congenital adrenal hyperplasia, diabetes, Von Hippel-Lindau (VHL) syndrome, pancreatitis, hyperparathyroidism, hypoparathyroidism, hyperthyroidism, hypothyroidism, SIDS, endometriosis, fertility, xerostomia, sclerodermna, hypercalceimia, ulcers, cirrhosis, inflammatory bowel disease, diverticular disease, Hirschsprung's disease, Crohn's Disease, appendicitis, hemophilia, hypercoagulation, idiopathic thrombocytopenic purpura, autoimmune disease, allergies, immunodeficiencies, transplantation, graft versus host disease, anemia, ataxia-telangiectasia, autoimmune disease, immunodeficiencies, hemophilia, hypercoagulation, idiopathic thrombocytopenic purpura, allergies, immunodeficiencies, transplantation, graft versus host disease (GVHD), lymphaedema, tonsilitis, hypogonadism, osteoporosis, hypercalcemia, arthritis, ankylosing spondylitis, scoliosis, arthritis, tendinitis, muscular dystrophy, Lesch-Nyhan syndrome, myasthenia gravis, dental disease, Alzheimer's disease, stroke, tuberous sclerosis, hypercalceimia, Parkinson's disease, Huntington's disease, cerebral palsy, epilepsy, multiple sclerosis, leukodystrophies, behavioral disorders, addiction, anxiety, pain, neurodegeneration, endocrine dysfunctions, diabetes, obesity, growth and reproductive disorders, multiple sclerosis, leukodystrophies, pain, neuroprotection, systemic lupus erythematosus, autoimmune disease, asthma, emphysema, scleroderma, allergy, ARDS, pharyngitis, laryngitis, diabetes, tuberous sclerosis, hearing loss, tinnitus, psoriasis, actinic keratosis, tuberous sclerosis, acne, hair growth/loss, allopecia, pigmentation disorders, endocrine disorders, psoriasis, actinic keratosis, tuberous sclerosis, acne, hair growth/loss, allopecia, pigmentation disorders, endocrine disorders, cystitis, incontinence, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, vesicoureteral refluxas well as other diseases, disorders and conditions.

The novel nucleic acid encoding the novel Neuropathy Target Esterase/Swiss Cheese protein-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV7 protein has multiple hydrophilic regions, each of which can be used as an immunogen. In one embodiment, a contemplated NOV7 epitope is from about amino acids 10 to 100. In another embodiment, a contemplated NOV7 epitope is from about amino acids 205 to 220. In other specific embodiments, contemplated NOV7 epitopes are from about amino acids 310 to 415, 510 to 520, 570 to 580, 700 to 800, 820 to 970, 1030 to 1210 and 1370 to 1410.

NOV8

A disclosed NOV8 nucleic acid (alternatively referred to herein as CG57119-01) encodes a novel Acid-Sensitive Potassium Channel Protein Task-like protein and includes the 815 nucleotide sequence (SEQ ID NO:23) shown in Table 8A. An open reading frame for the mature protein was identified beginning with an GTG codon at nucleotides 2-4 and ending with a TGA codon at nucleotides 638-640. Putative untranslated regions are underlined in Table 7A, and the start and stop codons are in bold letters.

TABLE 8A
NOV8 Nucleotide Sequence

(SEQ ID NO:23)

GGTGGGCGCTGCTGTCTTCGACGCGCTCGAGTCCGAGGCGGAAAGCGGCCGCCAGCGACTGCTGGTCCAGAAGCGG
GGCGCTCTCCGGAGGAAGTTCGGCTTCTCGGCCGAGGACTACCGCGAGCTGGAGCGCCTGGCGCTCCAGGCTGAGC
CCCACCGCGCCGGCCGCCAGTGGAAGTTCCCCGGCTCCTTCTACTTCGCCATCACCGTCATCACTACCATCGAGTA
CGGCCACGCCGCGCCGGGTACGGACTCCGGCAAGGTCTTCTGCATGTTCTACGCGCTCCTGGGCATCCCCCTGACG
CTGGTCACTTTCCAGAGCCTGGGCGAACGGCTGAACGCGGTCGTGCGGCGCCTCCTGTTGGCGGCCAAGTGCTGCC
TGGGCCTGCGGTGGACGTGCGTGTCCACGGAGAACCTGGTGGTGGCCGGGCTGCTGGCGTGTGCCGCCACCCTGGC
CCTCGGGGCCGTCGCCTTCTCGCACTTCGACGOCTGGACCTTCTTCCACGCCTACTACTACTGCTTCATCACCCTC
ACCACCATCGGCTTCGGCGACAACCTGGGCTTTTCGCCCCCCTCGAGCCCGGGGGTCGTGCGTGGCGGGCAGGCTC
CCAGGCTTGGGGCCCGGTGGAAGTCCATCTGACAACCCCACCCAGGCCAGGGTCGAATCTGGAATGGGAGGGTCTG
GCTTCAGCTATCAGGGCACCCTCCCCAGGGATTGGAAACGGATGACGGGCCTCTAGGCGGTCTTCTGCCACGAGCA
GTTTCTCATTACTGTCTGTGGCTAAGTCCCCTCCCTCCTTTCCAAAAATATATTA

The nucleic acid sequence of NOV8 has 556 of 560 bases (99%) identical to a gb:GENBANK-ID:AF257081|acc:AF257081.1 mRNA from Homo sapiens (Homo sapiens two pore potassium channel KT3.3 mRNA, complete cds) (E=5.6e⁻¹¹⁹).

A disclosed NOV8 polypeptide (SEQ ID NO:24) is 212 amino acid residues in length and is presented using the one-letter amino acid code in Table 8B. The SignalP, Psort and/or Hydropathy results predict that NOV8 does not have a signal peptide and is likely to be plasma membrane with a certainty of 0.6000. In alternative embodiments, a NOV8 polypeptide is located to the Golgi body with a certainty of 0.4000, the endoplasmic reticulum (membrane) with a certainty of 0.3000 or the mitochondrial inner membrane with a certainty of 0.1000.

TABLE 8B
Encoded NOV8 Protein Sequence

(SEQ ID NO:24)

VGAAVFDALESEAESGRQRLLVQKRGALRRKFGFSAEDYRELERLALQAEPHRAGRQWKFPGSFYFAITVITTI
EYGHAAPGTDSGKVFCMFYALLGIPLTLVTFQSLGERLNAVVRRLLLAAKCCLGLRWTCVSTENLVVAGLLACA
ATLALGAVAFSHFEGWTFFHAYYYCFITLTTIGFGDNLGFSPPSSPGVVRGGQAPRLGARWKSI

The NOV8 amino acid sequence was found to have 184 of 184 amino acid residues (100%) identical to, and 184 of 184 amino acid residues (100%) similar to, the 330 amino acid residue ptnr:TREMBLNEW-ACC:CAC14068 protein from Homo sapiens (Human) (DJ781B1.1 (A NOVEL PROTEIN SIMILAR TO THE ACID-SENSITIVE POTASSIUM CHANNEL PROTEIN TASK (KCNK3))) (E=8.8e⁻¹¹¹).

NOV8 is expressed in at least the following tissues: pancreas, placenta, brain, lung, prostate, heart, kidney, uterus, small intestine and colon. Expression information was derived from the tissue sources of the sequences that were included in the derivation of the sequence of NOV8.

Possible small nucleotide polymorphisms (SNPs) found for NOV8 are listed in Table 8C.

TABLE 8C
SNPs

| | Nucleotide | | Amino Acid | |
|---|---|---|---|---|
| Variant | Position | Base Change | Position | Base Change |
| 13376993 | 225 | A > G | 75 | Glu > Gly |
| 13376995 | 605 | G > A | 202 | Ala > Thr |
| 13376995 | 615 | T > C | 205 | Leu > Pro |

NOV8 also has homology to the amino acid sequences shown in the BLASTP data listed in Table 8D.

TABLE 8D
BLAST results for NOV8

| Gene Index/ | | Length | Identity | Positives | |
|---|---|---|---|---|---|
| Identifier | Protein/Organism | (aa) | (%) | (%) | Expect |
| gi\|10944275\|emb\|CAC14068.1\| | Two pore | 330 | 184/184 | 184/184 | 2e−88 |
| (AL118522) | potassium channel | | (100%) | (100%) | |
| dJ781B1.1 | KT3.3 (LOC64181) | | | | |
| | [Homo sapiens] | | | | |
| gi\|11641275\|ref\| | potassium family, | 330 | 183/184 | 183/184 | 1e−87 |
| NP_071753.1\| | subfamily K, | | (99%) | (99%) | |
| (NM_022358) | member 15; two | | | | |
| | pore potassium | | | | |
| | channel KT3.3; | | | | |
| | potassium | | | | |
| | channel, | | | | |
| | subfamily K, | | | | |
| | member 14 [Homo | | | | |
| | sapiens] | | | | |
| gi\|14771013\|ref\| | potassium | 330 | 183/184 | 183/184 | 2e−87 |
| XP_029815.1\| | channel, | | (99%) | (99%) | |
| (XM_029815) | subfamily K, | | | | |
| | member 14 [Homo | | | | |
| | sapiens] | | | | |
| gi\|7706135\|ref\| | potassium | 374 | 123/184 | 141/184 | 2e−65 |
| NP_057685.1\| | channel, | | (66%) | (75%) | |
| (NM_016601) | subfamily K, | | | | |
| | member 9; | | | | |
| | potassium channel | | | | |
| | TASK3; acid- | | | | |
| | sensitive | | | | |
| | potassium channel | | | | |
| | protein TASK-3; | | | | |
| | TWIK-related | | | | |
| | acid-sensitive K + | | | | |
| | channel 3 [Homo | | | | |
| | sapiens] | | | | |
| gi\|13431425\|sp\|Q | Potassium channel | 365 | 124/184 | 140/184 | 1e−64 |
| 9JL58\|CIW9_CAVPO | subfamily K | | (67%) | (75%) | |
| | member 9 (Acid- | | | | |
| | sensitive | | | | |
| | potassium channel | | | | |
| | protein TASK-3) | | | | |
| | (TWIK-related | | | | |
| | acid-sensitive K + | | | | |
| | channel 3) | | | | |

The homology of these sequences is shown graphically in the ClustalW analysis shown in Table 8E.

[image]

[image]

Duprat et al. (EMBO J. 1997;16:5464-71) identified TASK as a new member of the recently recognized TWIK K+ channel family. This 395 amino acid polypeptide has four transmembrane segments and two P domains. In adult human, TASK transcripts are found in pancreas<placenta<brain<lung, prostate<heart, kidney<uterus, small intestine and colon. Electrophysiological properties of TASK were determined after expression in Xenopus oocytes and COS cells. TASK currents are K+-selective, instantaneous and non-inactivating. They show an outward rectification when external [K+] is low ([K+]out=2 mM) which is not observed for high [K+]out (98 mM). The rectification can be approximated by the Goldman-Hodgkin-Katz current equation that predicts a curvature of the current-voltage plot in asymmetric K+ conditions. This strongly suggests that TASK lacks intrinsic voltage sensitivity. The absence of activation and inactivation kinetics as well as voltage independence are characteristic of conductances referred to as leak or background conductances. For this reason, TASK is designated as a background K+ channel. TASK is very sensitive to variations of extracellular pH in a narrow physiological range; as much as 90% of the maximum current is recorded at pH 7.7 and only 10% at pH 6.7. This property is probably essential for its physiological function, and suggests that small pH variations may serve a communication role in the nervous system.

Lesage et al. (EMBO J. 1996;15:1004-11) isolated a new human weakly inward rectifying K+ channel, TWIK-1. This channel is 336 amino acids long and has four transmembrane domains. Unlike other mammalian K+ channels, it contains two pore-forming regions called P domains. Genes encoding structural homologues are present in the genome of Caenorhabditis elegans. TWIK-1 currents expressed in Xenopus oocytes are time-independent and present a nearly linear I-V relationship that saturated for depolarizations positive to 0 mV in the presence of internal Mg2+. This inward rectification is abolished in the absence of internal Mg2+. TWIK-1 has a unitary conductance of 34 pS and a kinetic behavior that is dependent on the membrane potential. In the presence of internal Mg2+, the mean open times are 0.3 and 1.9 ms at −80 and +80 mV, respectively. The channel activity is up-regulated by activation of protein kinase C and down-regulated by internal acidification. Both types of regulation are indirect. TWIK-1 channel activity is blocked by Ba2+(IC50=100 microM), quinine (IC50=50 microM) and quinidine (IC50=95 microM). This channel is of particular interest because its mRNA is widely distributed in human tissues, and is particularly abundant in brain and heart. TWIK-1 channels are probably involved in the control of background K+ membrane conductances.

The first member of this family (TOK1) cloned from S.cerevisiae is predicted to have eight potential transmembrane (TM) helices. However, subsequently-cloned two P-domain family members from Drosophila and mammalian species are predicted to have only four TM segments. They are usually referred to as TWIK-related channels (Tandem of P-domains in a Weakly Inward rectifing K+ channel). Functional characterization of these channels has revealed a diversity of properties in that they may show inward or outward rectification, their activity may be modulated in different directions by protein phosphorylation, and their sensitivity to changes in intracellular or extracellular pH varies. Despite these disparate properties, they are all thought to share the same topology of four TM segments, including two P-domains. That TWIK-related K+ channels all produce instantaneous and non-inactivating K+ currents, which do not display a voltage-dependent activation threshold, suggests that they are background (leak) K+ channels involved in the generation and modulation of the resting membrane potential in various cell types. Further studies have revealed that they may be found in many species, including: plants, invertebrates and mammals.

TASK is a member of the TWIK-related (two P-domain) K+ channel family identified in human tissues. It is widely distributed, being particularly abundant in the pancreas and placenta, but it is also found in the brain, heart, lung and kidney. Its amino acid identity to TWIK-1 and TREK-1 is rather low, being about 25-28%. However, it is thought to share the same topology of four TM segments, with two P-domains. TASK is very sensitive to variations in extracellular pH in the physiological range, changing from fully-open to closed in approximately 0.5 pH units around pH 7.4. Thus, it may well be a biological sensor of external pH variations.

The protein similarity information, expression pattern, cellular localization, and map location for the protein and nucleic acid disclosed herein suggest that this Acid-Sensitive Potassium Channel Protein Task-like protein may have important structural and/or physiological functions characteristic of the Ion Channel family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: diabetes, Von Hippel-Lindau (VHL) syndrome, pancreatitis, obesity, fertility, Alzheimer's disease, stroke, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, systemic lupus erythematosus, autoimmune disease, asthma, emphysema, scleroderma, allergies, ARDS, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, transplantation, renal artery stenosis, interstitial nephritis, glomerulonephritis, polycystic kidney disease, renal tubular acidosis, IgA nephropathy, endometriosis, inflammatory bowel disease, diverticular disease, as well as other diseases, disorders and conditions.

The novel nucleic acid encoding the novel protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOVS protein has multiple hydrophilic regions, each of which can be used as an immunogen. In one embodiment, a contemplated NOV8 epitope is from about amino acids 20 to 30. In another embodiment, a contemplated NOV8 epitope is from about amino acids 41 to 45. In other specific embodiments, contemplated NOV8 epitopes are from about amino acids 49 to 55, 70 to 75 and 190 to 205.

NOV9

A disclosed NOV9 nucleic acid (designated as CuraGen Acc. No. CG57143-01), encodes a novel Ribosomal protein-like protein and includes the 711 nucleotide sequence (SEQ ID NO:25) shown in Table 9A. An open reading frame for the mature protein was identified beginning with an ATG codon at nucleotides 44-46 and ending with a TAG codon at nucleotides 674-676. The start and stop codons are in bold letters in Table 9A.

TABLE 9A
NOV9 Nucleotide Sequence

(SEQ ID NO:25)

TCTCTCTCTCTCTCTCTCTCTCTGGTGAACAGGACCCGTCGCCATGGGCCGTGTGATCCGTGGACAGAGGAAGGG
CGCCGGGTCTGTGTTCCGCGCGCACGTGAAGCACCGTAAAGGCGCTGCGCGCCTGCGCGCCGTGGATTTCGCTGA
GCGGCACGGCTACATCAAGGGCATCGTCAAGGCCCAGCTCAACATTGGCAATGTGCTCCCTGTGGGCACCATGCC
TGAGGGTACAATCGTGTGCTGCCTGGAGGAGAAGCCTGGAGACCGTGGCAAGCTGGCCCGGGCATCAGGGAACTA
TGCCACCGTTATCTCCCACAACCCTGAGACCAAGAAGACCCGTGTGAAGCTGCCCTCCGGCTCCAAGAAGGTTAT
CTCCTCAGCCAACAGAGCTGTGGTTGGTGTGGTGGCTGGAGGTGGCCGAATTGACAAACCCATCTTGAAGGCTGG
CCGGGCGTACCACAAATATAAGGCAAAGAGGAACTGCTGGCCACGAGTACGGGGTGTGGCCATGAATCCTGTGGA
GCATCCTTTTGGAGGTGGCAACCACCAGCACATCGGCAAGCCCTCCACCATCCGCAGAGATGCCCCTGCTGGCCG
CAAAGTGGGTCTCATTGCTGCCCGCCGGACTGGACGTCTCCGGGGAACCAAGACTGTGCAGGAGAAAGAGAACTA
GTGCTGAGGGCCTCAATAAAGTTTGTGTTTATGCCA

The nucleic acid sequence of NOV9 maps to chromosome 8 and has invention has 574 of 610 bases (94%) identical to a gb:GENBANK-ID:HSRBPL8|acc:Z28407.1 mRNA from Homo sapiens (H.sapiens mRNA for ribosomal protein L8) (E=9.9e⁻¹¹⁵)

The NOV9 polypeptide (SEQ ID NO:26) is 210 amino acid residues in length and is presented using the one-letter amino acid code in Table 9B. The SignalP, Psort and/or Hydropathy results predict that NOV9 does not have a signal peptide and is likely to be localized to the nucleus with a certainty of 0.9749. In alternative embodiments, a NOV9 polypeptide is located to the mitochondrial matrix space with a certainty of 0.4248, the 110 microbody (peroxisome) with a certainty of 0.3000, or the lysosome (lumen) with a certainty of 0.2783.

TABLE 9B
Encoded NOV9 Protein Sequence

(SEQ ID NO:26)

MGRVIRGQRKGAGSVFRAHVKHRKGAARLRAVDFAERHGYIKGIVKAQLNIGNVLPVGTMPEGTIVCCLEEKPG
DRGKLARASGNYATVISHNPETKKTRVKLPSGSKKVISSANRAVVGVVAGGGRIDKPILKAGRAYHKYKAKRNC
WPRVRGVAMNPVEHPFGGGNHQHIGKPSTIRRDAPAGRKVGLIAARRTGRLRGTKTVQEKEN

The NOV9 amino acid sequence was found to have 170 of 196 amino acid residues (86%) identical to, and 175 of 196 amino acid residues (89%) similar to, the 257 amino acid residue ptnr:SWISSNEW-ACC:P25120 protein from Homo sapiens (Human), Rattus norvegicus (Rat), and (60S RIBOSOMAL PROTEIN L8) (E=12e⁻⁸⁶).

NOV9 is expressed in at least the following tissues: granulosa cells, white blood cells, bone marrow, liver, lung, placenta and whole organism. Expression information was derived from the tissue sources of the sequences that were included in the derivation of the sequence of NOV9.

Possible small nucleotide polymorphisms (SNPs) found for NOV9 are listed in Table 9C.

TABLE 9C
SNPs

| | Nucleotide | | Amino Acid | |
|---|---|---|---|---|
| Variant | Position | Base Change | Position | Base Change |
| 13376997 | 152 | C > T | 37 | Arg > Trp |
| 13376996 | 611 | C > T | 190 | Leu > Phe |

NOV9 also has homology to the amino acid sequences shown in the BLASTP data listed in Table 9D.

TABLE 9D
BLAST results for NOV9

| Gene Index/ | Protein/ | Length | Identity | Positives | |
|---|---|---|---|---|---|
| Identifier | Organism | (aa) | (%) | (%) | Expect |
| gi\|730576\|sp\| | 60S RIBOSOMAL | 257 | 204/257 | 210/257 | 2e−92 |
| P41116\|RL8_{—} | PROTEIN L8 | | (79%) | (81%) | |
| XENLA | | | | | |
| gi\|4506663\|ref\| | ribosomal | 257 | 210/257 | 210/257 | 2e−89 |
| NP_000964.1\| | protein L8; | | (81%) | (81%) | |
| (NM_000973) | 60S ribosomal | | | | |
| | protein L8 | | | | |
| | [Homo | | | | |
| | sapiens] | | | | |
| gi\|15082586\|gb\| | Similar to | 257 | 209/257 | 210/257 | 3e−89 |
| AAH12197.1\| | ribosomal | | (81%) | (81%) | |
| AAH12197 | protein L8 | | | | |
| (BC012197) | [Homo | | | | |
| | sapiens] | | | | |
| gi\|15293881\|gb\| | ribosomal | 257 | 198/257 | 204/257 | 3e−86 |
| AAK95133.1\| | protein L8 | | (77%) | (79%) | |
| AF401561_1 | [Ictalurus | | | | |
| (AF401561) | punctatus] | | | | |
| gi\|12652605\|gb\| | Similar to | 214 | 170/196 | 175/196 | 3e−75 |
| AAH00047.1\| | ribosomal | | (86%) | (88%) | |
| AAH00047 | protein L8 | | | | |
| (BC000047) | [Homo | | | | |
| | sapiens] | | | | |

The homology of these sequences is shown graphically in the ClustalW analysis shown in Table 9E.

[image]

[image]

Table 9F lists the domain description from DOMAIN analysis results against NOV9. This indicates that the NOV9 sequence has properties similar to those of other proteins known to contain these domains.

TABLE 9F
Domain Analysis of NOV9

gnl|Pfam|pfam00181, Ribosoinal_L2, Ribosomal Proteins L2.
CD-Length = 229 residues, 100.0% aligned
Score = 177 bits (450), Expect = 4e-46

| | | | | |
|---|---|---|---|---|
| NOV9: | 13 | GSVFRAHVKHRKGAA----RLRAVDFAERHGYIKGIVK---------------------- | 46 | (SEQ ID NO:201) |
| | | \| \| \|+ \| \|\|+\|\| \| \|\|\|\|\| \|\| | | |
| Sbj | 1 | GRNNRGHITRRHRGGGHKRLYRAIDFKRRKGYIKGTVKRIEYDPNRSAPIALVVYSDPGE | 60 | (SEQ ID NO:202) |
| NOV9: | 47 | ---------------------AQLNIGNVLPVGTMPEGTIVCCLEEKPGDRGKLARASGN | 85 | |
| | | \| + \|\|\|\|\|\|+\| +\|\|\|\|\|+ +\|\|\|\|\|\| \|+\|\|\|\|+\| | | |
| Sbj | 61 | KRYILAPEGLHVGDTIYSGKNATIKIGNVLPLGEIPEGTIIHNVEEKPGDGGQLARAAGT | 120 | |
| NOV9: | 86 | YATVISHNPETKKTRVKLPSGSKKVISSANRAVVGVVAGGGRIDKPILKAGRAYHKYKAK | 145 | |
| | | \|\| +++\|+ + \|\|\|\|\|\|\|\|\|\| \|+ +\|\| \|\| +\|\|\|\| \|\|\|\|\|\|\|+ \|\|\|\|\| ++ \| | | |
| Sbj: | 121 | YAQILAHDGD-KKTRVKLPSGEKRRVSSECRATIGVVANGGRIDKPLGKAGRA--RWLGK | 177 | |
| NOV9: | 146 | RNCWPRVRGVAMNPVEHPFGGGNHQHIGKPSTIRRDAPAGRKVGLIAARRTGRLRGT | 202 | |
| | | \| \|\|\|\|\|\|\|\|\|\|\|+\|\| \|\|\| +\| \| \|+\| \|\| \|\|\| \|\| | | |
| Sbj: | 178 | R---PRVRGVAMNPVDHPHGGGEGRHP--IGRKSPVTPWGKKALGIATRRTKRLSDK | 229 | |

The mammalian ribosome is composed of 4 RNA species (see 180450) and approximately 80 different proteins (see 180466).

The rat ribosomal protein L8 (Rp18) associates with 5.8S rRNA, very likely participates in the binding of aminoacyl-tRNA, and has been identified as a constituent of the EF2 (130610)-binding site at the ribosomal subunit interface. By screening a human ovarian granulosa cell cDNA expression library with antibodies against human follicular fluid glycoproteins, Hanes et al. (1993) isolated a partial RPL8 cDNA. They completed the full-length cDNA sequence using PCR. The deduced 257-amino acid human RPL8 protein is identical to rat Rp18. Northern blot analysis detected a 900-bp RPL8 transcript in human granulosa cells and white blood cells. By somatic cell hybrid and radiation hybrid mapping analyses, Kenmochi et al. (1998) mapped the human RPL8 gene to 8q.

Ribosomal_L2 (Ribosomal Proteins L2), amino acid 13 to 46 and 47 to 210.

Ribosomal protein L2 is one of the proteins from the large ribosomal subunit. In Escherichia coli, L2 is known to bind to the 23S rRNA and to have peptidyltransferase activity. It belongs to a family of ribosomal proteins which, on the basis of sequence similarities, groups: Eubacterial L2, Algal and plant chloroplast L2, Cyanelle L2, Archaebacterial L2, Plant L2, Slime mold L2, Marchantia polymorpha mitochondrial L2, Paramecium tetraurelia mitochondrial L2, Fission yeast K5, K37 and KD4, Yeast YL6, Vertebrate L8. See Interpro IPR002171:

The protein similarity information, expression pattern, cellular localization, and map location for the protein and nucleic acid disclosed herein suggest that this Ribosomal Protein-like protein may have important structural and/or physiological functions characteristic of the Ribosomal Proteins family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: hemophilia, hypercoagulation, idiopathic thrombocytopenic purpura, autoimmune disease, allergies, asthma, immunodeficiencies, transplantation, graft versus host disease, Von Hippel-Lindau (VHL) syndrome, cirrhosis, systemic lupus erythematosus, emphysema, scleroderma, ARDS, fertility as well as other diseases, disorders and conditions.

The novel nucleic acid encoding the novel Ribosomal Protein-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV9 protein has multiple hydrophilic regions, each of which can be used as an immunogen. In one embodiment, a contemplated NOV9 epitope is from about amino acids 10 to 15. In another embodiment, a contemplated NOV9 epitope is from about amino acids 40 to 42. In other specific embodiments, contemplated NOV9 epitopes are from about amino acids 55 to 57, 70 to 75, 90 to 95, 99 to 110, 135 to 150, 155 to 175, 180 to 183, 190 to 193 and 199 to 201.

NOV10

A disclosed NOV10 is nucleic acid (designated as CuraGen Acc. No. CG56860-01, encodes a novel Prostaglandin Omega Hydroxylase-like protein and includes the 1503 nucleotide sequence (SEQ ID NO:27) shown in Table 10A. An open reading frame for the mature protein was identified beginning with an ATG codon at nucleotides 11-14 and ending with a TAG codon at nucleotides 1493-1495. Putative untranslated regions downstream from the termination codon are underlined in Table 10A, and the stop codon is in bold letters.

TABLE 10A
NOV10 Nucleotide Sequence

(SEQ ID NO:27)

GTGCTGCGGCATGAGTGTCTCTGTGCTGAACCCCAACAGACTCCCAGATGGTGTCTCAGGGCTCCTCCAAGGAGC
CTCACTGCTGAGCCTGCTTCTGTTACTATTGAAGGCAGCCCAGCCCTACCTGCGGAGGCAGCGGCTGCTGCGGGA
CCTGCGCCCCTTCCCAGCGCCCCCCACCCACTGGTTCCTTGGGCACAAGCTGATGGAAAAATACCCATGTGCTGT
TCCCTTGTGGGTTGGACCCTTTACGATGTTCTTCAGTGTCCATGACCCAGACTATGCCAAGATTCTCCTGAAAAG
ACAAGGTAAAAACCAAGAGGGGTTTCTGCCTTTTATTTCTCAAGGAAAAGGACTAGCGGCTCTAGACGGAGGGAA
GTGGTTCCAGCATCGTCGCCTACTAACTCCTGGATTCCATTTTAACATCCTGAAAGCATACATTGAGGTGATGGC
TCATTCTGTGAAAATGATGCTGAACAAATGGGAGGAACACATTGCCCAAAACTCACGTCTGGAGCTCTTTCAACA
TGTCTCCCTGATGACCCTGGACAGCATCATGAAGTGTGCCTTCAGCCACCAGGGCAGCATCCAGTTGGACAGGTC
ATCATACCTGAAAGCAGTGTTCAACCTTAGCAAAATCTCCAACCAGCGCATGAACAATTTTCTACATCACAACGA
CCTGGTTTTCAAATTCAGCTCTCAAGGCCAAATCTTTTCTAAATTTAACCAAGAACTTCATCAGCATCTAGAGAA
AGTAATCCAGGACCGGAAGGAGTCTCTTAAGGATAAGCTAAAACAAGATACTACTCAGAAAAGGCGCTGGGATTT
TCTGGACATACTTTTGAGTGCCAAAGTAGAAAACACCAAAGATTTCTCTGAAGCAGATCTCCAGGCTGAAGTGAA
AACGTTCATGTTTGCAGGACATGACACCACATCCAGTGCTATCTCCTGGATCCTTTACTGCTTGGCAAAGTACCC
TGAGCATCAGCAGAGATGCCGAGATGAAATCAGGGAACTCCTAGGGGATGGGTCTTCTATTACCTGGCACCTGAG
CCAGATGCCTTACACCACGATGTGCATCAAGGAATGCCTCCGCCTCTACGCACCGGTAGTAAACATATCCCGGTT
ACTCGACAAACCCATCACCTTTCCAGATGGACGCTCCTTACCTGCAGGGATCACCGTGGTTCTTAGTATTTGGGG
TCTTCACCACAACCCTGCTGTCTGGAAAAACGTACAGGTCTTTGACCCCTTGAGGTTCTCTCAGGAGAATTCTGA
TCAGAGACACCCCTATGCCTACTTACCATTCTCAGCTGGATCAAGGAACTGCATTGGGCAGGAGTTTGCCATGAT
TGAGTTAAAGGTAACCATTGCCTTGATTCTGCTCCACTTCAGAGTGACTCCAGACCCCACCAGGCCTCTTACTTT
CCCCAACCATTTTATCCTCAAGCCCAAGAATGGGATGTATTTGCACCTGAAGAAACTCTCTGAATGTTAGATCTC
AGG

The nucleic acid sequence of NOV10 maps to chromosome 1 and has 525 of 755 bases (69%) identical to a gb:GENBANK-ID:HUMCYTFAOH|acc:L04751.1 mRNA from Homo sapiens (Human cytochrome p-4504A (CYP4A) mRNA, complete cds) (E=1.6e⁻¹¹⁶).

A disclosed NOV10 polypeptide (SEQ ID NO:28) is 494 amino acid residues in length and is presented using the one-letter amino acid code in Table 10B. The SignalP, Psort and/or Hydropathy results predict that NOV10 has a signal peptide and is likely to be localized to the plasma membrane with a certainty of 0.6000. In alternative embodiments, a NOV10 polypeptide is located to the Golgi body with a certainty of 0.4000, the endoplasmic reticulum (membrane) with a certainty of 0.3000, or the microbody (peroxisome) with a certainty of 0.3000. The SignalP predicts a likely cleavage site for a NOV10 peptide between amino acid positions 35 and 36, ie. at the sequence KAA-QP.

TABLE 10B
Encoded NOV10 Protein Sequence

(SEQ ID NO:28)

MSVSVLNPNRLPDGVSGLLQGASLLSLLLLLLKAAQPYLRRQRLLRDLRPFPAPPTHWFLGHKLMEKYPCAVP
LWVGPFTMFFSVHDPDYAKILLKRQGKNQEGFLPFISQGKGLAALDGPKWFQHRRLLTPGFHFNILKAYIEVM
AHSVKMMLNKWEEHIAQNSRLELFQHVSLMTLDSIMKCAFSHQGSIQLDRSSYLKAVFNLSKISNQRMNNFLH
HNDLVFKFSSQGQIFSKFNQELHQHLEKVIQDRKESLKDKLKQDTTQKRRWDFLDILLSAKVENTKDFSEADL
QAEVKTFMFAGHDTTSSAISWILYCLAKYPEHQQRCRDEIRELLGDGSSITWHLSQMPYTTMCIKECLRLYAP
VVNISRLLDKPITFPDGRSLPAGITVVLSIWGLHHNPAVWKNVQVFDPLRFSQENSDQRHPYAYLPFSAGSRN
CIGQEFAMIELKVTIALILLHFRVTPDPTRPLTFPNHFILKPKNGMYLHLKKLSEC

The NOV10 amino acid sequence was found to have 281 of 509 amino acid residues (55%) identical to, and 369 of 509 amino acid residues (72%) similar to, the 510 amino acid residue ptnr:pir-id:A29368 protein from rabbit (prostaglandin omega-hydroxylase (EC 1.14.15.-) cytochrome P4504A4) (E=1.7e⁻¹⁴⁴).

NOV10 is expressed in at least the following tissues: Brain, Substantia Nigra, Hippocampus, Hypothalamus, Kidney, Lung, Mammary gland/Breast, Parietal Lobe, Prostate, and Uterus. Expression information was derived from the tissue sources of the sequences that were included in the derivation of the sequence of NOV10.

NOV10 also has homology to the amino acid sequences shown in the BLASTP data listed in Table 10C.

TABLE 10C
BLAST results for NOV10

| Gene Index/ | Protein/ | Length | Identity | Positives | |
|---|---|---|---|---|---|
| Identifier | Organism | (aa) | (%) | (%) | Expect |
| gi\|2493371\|sp\|Q02928\| | (FATTY ACID | 519 | 282/511 | 358/511 | e−146 |
| CP4Y_HUMAN | OMEGA- | | (55%) | (69%) | |
| CYTOCHROME P450 | HYDROXYLASE) (P- | | | | |
| 4A11 PRECURSOR | 450 HK OMEGA) | | | | |
| (CYPIVA11) | (LAURIC ACID | | | | |
| | OMEGA- | | | | |
| | HYDROXYLASE) | | | | |
| | (CYP4AII) (P450- | | | | |
| | HL-OMEGA) | | | | |
| gi\|203787\|gb\|AAA41038.1\| | cytochrome P-450 | 509 | 269/511 | 357/511 | e−145 |
| (M57718) | IVA1 [Rattus | | (52%) | (69%) | |
| | norvegicus] | | | | |
| gi\|12832576\|dbj\| | cytochrome P450, | 509 | 271/512 | 357/512 | e−145 |
| BAB22165.1\| | 4a10˜data | | (52%) | (68%) | |
| (AK002528) | source: MGD, | | | | |
| | source | | | | |
| | key: MGI: 88611, | | | | |
| | evidence: ISS˜put | | | | |
| | ative [Mus | | | | |
| | musculus] | | | | |
| gi\|3738263\|dbj\|BAA33804.1\| | cytochrome P-450 | 509 | 271/512 | 357/512 | e−145 |
| (AB018421) | [Mus musculus] | | (52%) | (68%) | |
| gi\|4503235\|ref\|NP_000769.1\| | cytochrome P450, | 519 | 282/511 | 358/511 | e−145 |
| (NM_000778) | subfamily IVA, | | (55%) | (69%) | |
| | polypeptide 11; | | | | |
| | fatty acid | | | | |
| | omega- | | | | |
| | hydroxylase; | | | | |
| | P450HL-omega; | | | | |
| | alkane-1 | | | | |
| | monooxygenase; | | | | |
| | lauric acid | | | | |
| | omega- | | | | |
| | hydroxylase | | | | |
| | [Homo sapiens] | | | | |

The homology of these sequences is shown graphically in the ClustalW analysis shown in Table 10D.

[image]

[image]

Table 10E lists the domain description from DOMAIN analysis results against NOV110. This indicates that the NOV110 sequence has properties similar to those of other proteins known to contain these domains.

TABLE 9E
Domain Analysis of NOV10

gnl|Pfam|pfam00067, p450, Cytochrome P450. Cytochrome P450s are involved in
the oxidative degradation of various compounds. Particularly well known for
their role in the degradation of environmental toxins and mutagens. Structure
is mostly alpha, and binds a heme cofactor.
CD-Length = 445 residues, 98~9% aligned
Score = 304 bits (778), Expect = 9e-84

| | | | | |
|---|---|---|---|---|
| NOV10: | 52 | PAPPTHWFLGH-------------KLMEKYPCAVPLWVGPFTMFFSVHDPDYAKILLKRQ | 98 | (SEQ ID NO:208) |
| | | \| \|\| +\|+ +\| +\|\| \|++\|\| + \| \|+ \| +\| + | | |
| Sbjct: | 2 | PGPPPLPLIGNLLQLGRGPIHSLTELRKKYGPVFTLYLGPRPVVV-VTGPEAVKEVLIDK | 60 | (SEQ ID NO:209) |
| NOV10: | 99 | GKNQEGFLPFISQ---GKGLAALDGPKWFQHRRLLTPGFHFNILKAYIEVMAHSVKMMLN | 155 | |
| | | \|+ \| \| \| \|+ +\|\|+\| \| \|\|\|\|\| \| \| + \| ++ + + | | |
| Sbjct: | 61 | GEEFAGRGDFPVFPWLGYGILFSNGPRWRQLRRLLTLRF-FGMGKRS-KLEERIQEEARD | 118 | |
| NOV10: | 156 | KWEE-HIAQNSRLELFQHVSLMTLDSIMKCAFSHQGSIQLDRSSYLKAVFNLSKISNQRM | 214 | |
| | | \| \| \| +++ + ++ \|+ \| \| + +\|\| + \|++ + | | |
| Sbjct: | 119 | LVERLRKEQGSPIDITELLAPAPLNVICSLLFGV--RFDYEDPEFLKLIDKLNE-LFFLV | 175 | |
| NOV10: | 215 | NNFLHHNDLVFKFSSQGQIFSKFNQELHQHLEKVIQDRKESLKDKLKQDTTQKRRWDFLD | 274 | |
| | | + + \| + \| ++\| +\|+\|+\|++\|+\|+\|+ \| \|\|\|\| | | |
| Sbjct: | 176 | SPWGQLLDFFRYLPGSHRKAFKAAKDLKDYLDKLIEERRETLE---PGDPR-----DFLD | 227 | |
| NOV10: | 275 | ILL-SAKVENTKDFSEADLQAEVKTFMFAGHDTTSSAISWILYCLAKYPEHQQRCRDEIR | 333 | |
| | | \|\| \|\| \| + ++ +\|+\| \| +\|\|\| \|\|\|\|\| +\|\| \|\| \|\|\|+\|\| \| + \|+\|\| | | |
| Sbjct: | 228 | SLLIEAKREGGSELTDEELKATVLDLLFAGTDTTSSTLSWALYLLAKHPEVQAXLREEID | 287 | |
| NOV10: | 334 | ELLGDGSSITW-HLSQMPYTTMCIKECLRLYAPVV-NISRLLDKPITFPDGRSLPAGITV | 391 | |
| | | \|++\| \| \|+ + \|\|\| \|\|\| \|\|\|+ \| + \|+ + \|\| +\| \| \| | | |
| Sbjct: | 288 | EVIGRDRSPTYDDRANMPYLDAVIKETLRLHPVVPLLLPRVATEDTEI-DGYLIPKGTLV | 346 | |
| NOV10: | 392 | VLSIWGLHHNPAVWKNVQNFDPLRFSQENSDQRHPYAYLPFSAGSRNCIGQEFAMIELKV | 451 | |
| | | +++++ \|\| +\| \|+ \| + \|\|\| \|\| \|\| + \|\|+\|\|\| \|\| \|\|\|+\|+ \| +\|\| + | | |
| Sbjct: | 347 | IVNLYSLHRDPKVFPNPEEFDPERFLDENGKFKKSYAFLPFGAGPRNCLGERLARMELFL | 406 | |
| NOV10: | 452 | TIALILLHFRV-TPDPTRPLTFPNHFILKPKNGMY | 485 | |
| | | +\| +\| \| + \| \| \| \| +\| | | |
| Sbjct: | 407 | FLATLLQRFELELVPPGDIPLTPKPLGLPSKPPLY | 441 | |

P4504A4 is a cytochrome P450 that is elevated during pregnancy. This P-450 isozyme regiospecifically hydroxylates PGE1, PGA1, and PGF2 alpha at carbon-20 (the omega position). This enzyme catalyzes the hydroxylation of PGA1 in the presence of NADPH.

The protein similarity information, expression pattern, cellular localization, and map location for the NOV10 protein and nucleic acid disclosed herein suggest that this prostaglandin omega-hydroxylase-like protein may have important structural and/or physiological functions characteristic of the PG omega/omega-1 hydroxylase family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Systemic lupus erythematosus, Autoimmune disease, Asthma, Emphysema, Scleroderma, allergy, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia as well as other diseases, disorders and conditions.

The novel nucleic acid encoding the Prostaglandin Omega Hydroxylase-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV10 protein has multiple hydrophilic regions, each of which can be used as an immunogen. In one embodiment, a contemplated NOV10 epitope is from about amino acids 40 to 50. In another embodiment, a contemplated NOV10 epitope is from about amino acids 51 to 55. In other specific embodiments, contemplated NOV10 epitopes are from about amino acids 100 to 102, 105 to 106, 130 to 132, 140 to 143, 160 to 165, 190 to 215, 240 to 265, 290 to 295, 330 to 340, 370 to 373, 410 to 440 and 470 to 490.

NOV11

The disclosed NOV11 nucleic acid (designated as CuraGen Acc. No. CG57024-01), encodes a novel Myeloid Upregulated Protein-like protein and includes the 1408 nucleotide sequence (SEQ ID NO:29) shown in Table 11A. An open reading frame for the mature protein was identified beginning with an ATG codon at nucleotides 153-155 and ending with a TGA codon at nucleotides 1185-1187. Putative untranslated regions downstream from the termination codon and upstream from the initiation codon are underlined in Table 11 A, and the start and stop codons are in bold letters.

TABLE 11A
NOV11 Nucleotide Sequence

(SEQ ID NO:29)

AGCAGAGAGGCTGCCCTGCTGCAATGTCACCGTCGTCACTGCCTCTGCAGGCTGCAGGCACCTGCCACTACCGCAG
AGGACTGAGGGGCCTTGGCCCAGCAGGGACCCCAGGGCCTTGGGGGACTGTGTGAGCTGGAAACGTGGCTGGCCAG
ATGGGCAGCACCATGGAGCCCCCTGGGGGTGCGTACCTGCACCTGGGCGCCGTGACATCCCCTGTGTGCACAGCCC
GCGTGCTGCAGCTGGCCTTTGGCTGCACTACCTTCAGCCTGGTGGCCCACCGGGGTGGCTTTGCGGGCGTCCAGGG
CACCTTCTGCATGGACGCCTGGGGCTTCTGCTTCGCCGTCTCTGCGCTGGTGGTGGCCTGTGAGTTCACACGGCTC
CACGGCTGCCTGCGGCTCTCCTGGGGCAACTTCACCGCCGCCTTCGCCATGCTGGCCACCCTGCTATGCGCGACGG
CTGCGGTCCTGTATCCGCTGTACTTTGCCCGGCGGGAGTGTTCCCCCGAGCCCGCCGGCTGTGCTGCCAGGGACTT
CCGCCTGGCAGCCAGTGTCTTCGCCGGGCTCCTCTTCCTGGCCTACGCTGTGGAGGTGGCCCTGACGCGGGCCCGG
CCCGGCCAGGTGAGCAGCTATATGGCCACGGTGTCGGGGCTCCTCAAGATCGTCCAGGCCTTCGTGGCCTGCATCA
TCTTCGGGGCGCTGGTCCATGACAGCCGCTACGGGCGCTACGTGGCCACCCAGTGGTGCGTGGCCGTCTACAGCCT
GTGCTTCCTGGCCACAGTGGCCGTGGTGGCCCTGAGTGTGATGGGCCACACAGGGGGCCTGGGCTGCCCCTTTGAC
CGGCTGGTGGTGGTGTACACCTTCCTGGCTGTGCTCCTGTACCTCAGCGCCGCCGTGATCTGGCCAGTCTTCTGTT
TCGATCCCAAGTACGGTGAGCCCAAACGGCCCCCCAACTGTGCTCGGGGCAGCTGTCCCTGGGACACCAGCTGGTG
GTGGCCATCTTCACCTACGTCAACCTGCTCCTGTACGTCGTTGACCTCGCCTACTCCCAGCTTCAGCAGTGCCCGG
CGGGCATCTGTGCACTGTGGGCATCTGTGGCACTGGGAGGGAGCCCGGCTGAGGGCGGCCGCTGGACACAGAATCT
GGGTACTGCTTGCCTCTGCTCAAGGGTCCAGTTGCCGAAACTCCTGACGCCGGGGCCATCATCCTCCAGGCTCCAG
CCAGCTTCTCCTGCACAGAAGCCCAGCCTGGTCCAGCCAGGAGCTGACCCACTGGCCACCCCTGAGTCCAAGCCGG
GTGGGCAGTGGCACAACAGCCCCTCAGCCCATTGACTGGGCCCCATTGACGTCCTTGAGCAGGAAATAAATGCTGA
CATTTATACGTACCCTGCCTCTGGACCAGCAGTCTCTTCT

The nucleic acid sequence of NOV11 maps to chromosome 2. A disclosed NOV11 polypeptide (SEQ ID NO:30) is 344 amino acid residues in length and is presented using the one-letter amino acid code in Table 11B. The SignalP, Psort and/or Hydropathy results predict that NOV11 is likely to be localized with a certainty of 0.7480. In alternative embodiments, a NOV11 polypeptide is located to the plasma membrane with a certainty of 0.7000, the endoplasmic reticulum (membrane) with a certainty of 0.2000, or the mitochondrial inner membrane with a certainty of 0.1000. The SignalP predicts a likely cleavage site for a NOV9 peptide between amino acid positions 33 and 34, i.e. at the sequence AFG-CT.

TABLE 11B
Encoded NOV11 Protein Sequence

(SEQ ID NO:30)

MGSTMEPPGGAYLHLGAVTSPVCTARVLQLAFGCTTFSLVAHRGGFAGVQGTFCMDAWGFCFAVSALVVACEFTRL
HGCLRLSWGNFTAAFAMLATLLCATAAVLYPLYFARRECSPEPAGCAARDFRLAASVFAGLLFLAYAVEVALTRAR
PGQVSSYMATVSGLLKIVQAFVACIIFGALVHDSRYGRYVATQWCVAVYSLCFLATVAVVALSVMGHTGGLGCPFD
RLVVVYTFLAVLLYLSAAVIWPVFCFDPKYGEPKRPPNCARGSCPWDTSWWWPSSPTSTCSCTSLTSPTPSFSSAR
RASVHCGHLWHWEGARLRAAAGHRIWVLLASAQGSSCRNS

The NOV11 amino acid sequence was found to have 92 of 226 amino acid residues (40%) identical to, and 127 of 226 amino acid residues (56%) similar to, the 296 amino acid residue ptnr:SWISSPROT-ACC:035682 protein from Mus musculus (Mouse) (MYELOID UPREGULATED PROTEIN) E=1.6e⁻³⁸).

NOV11 is expressed in at least the lung. Expression information was derived from the tissue sources of the sequences that were included in the derivation of the sequence of NOV11.

NOV11 also has homology to the amino acid sequences shown in the BLASTP data listed in Table 11C.

TABLE 11C
BLAST results for NOV11

| Gene Index/ | Protein/ | Length | Identity | Positives | |
|---|---|---|---|---|---|
| Identifier | Organism | (aa) | (%) | (%) | Expect |
| gi\|12834438\|dbj\| | evidence: NAS˜ | 153 | 110/122 | 113/122 | 4e−51 |
| BAB22911.1\|(AK003645) | hypothetical | | (90%) | (92%) | |
| | protein˜putative | | | | |
| | [Mus | | | | |
| | musculus] | | | | |
| gi\|17482569\|ref\| | hypothetical | 322 | 106/266 | 153/266 | 5e−38 |
| XP_039907.2\|(XM_039907) | protein | | (39%) | (56%) | |
| | XP_039907 | | | | |
| | [Homo | | | | |
| | sapiens] | | | | |
| gi\|8393800\|ref\| | myeloid- | 296 | 92/226 | 127/226 | 1e−29 |
| NP_058665.1\|(NM_016969) | associated | | (40%) | (55%) | |
| | differentiation | | | | |
| | marker [Mus | | | | |
| | musculus] | | | | |
| gi\|16553192\|dbj\| | unnamed protein | 245 | 74/178 | 106/178 | 2e−24 |
| BAB71502.1\|(AK057470) | product [Homo | | (41%) | (58%) | |
| | sapiens] | | | | |
| gi\|17445253\|ref\| | similar to | 331 | 86/243 | 127/243 | 1e−23 |
| XP_065813.1\|(XM_065813) | hypothetical | | (35%) | (51%) | |
| | protein SB135 | | | | |
| | [Homo | | | | |
| | sapiens] | | | | |

The homology of these sequences is shown graphically in the ClustalW analysis shown in Table 11D.

[image]

[image]

The protein encoded by NOV11 has high homology to mouse myeloid upregulated protein. It is a multipass trans-membrane protein. Since myeloid cells are critical players in inflammation and immune responses, this invention is an excellent antibody target to treat inflammation and immune disorders or as a diagnostic marker.

The protein similarity information, expression pattern, cellular localization, and map location for the NOV11 protein and nucleic acid disclosed herein suggest that this Myeloid Upregulated Protein-like protein may have important structural and/or physiological functions characteristic of the Mal family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: systemic lupus erythematosus, autoimmune disease, asthma, emphysema, scleroderma, allergy, ARDS, as well as other diseases, disorders and conditions.

The novel nucleic acid encoding Myeloid Upregulated Protein-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV11 protein has multiple hydrophilic regions, each of which can be used as an immunogen. In one embodiment, a contemplated NOV11 epitope is from about amino acids 5 to 90. In another embodiment, a contemplated NOV11 epitope is from about amino acids 105 to 110. In other specific embodiments, contemplated NOV11 epitopes are from about amino acids 170 to 180, 230 to 310, 370 to 400, 420 to 430, 450 to 455, 460 to 465, 480 to 485, 510 to 515, 570 to 580 and 680 to 690.

NOV12

A disclosed NOV12 nucleic acid (designated CuraGen Acc. No. CG57083-01) encodes a novel Testicular Serine Protease-like protein and includes the 1113 nucleotide sequence (SEQ ID NO: 31) which is shown in Table 12A. An open reading frame was identified beginning with an ATG initiation codon at nucleotides 1-3 and ending with a TGA codon at nucleotides 1069-1071. The start and stop codons are in bold letters and the untranslated regions are underlined in Table 12A.

TABLE 12A
NOV12 Nucleotide Sequence

(SEQ ID NO:31)

ATGGCCGAAGGTGAAGGGGAAGCAAGCACATCTTCACATGGTGACGGGAGAGAGAAAGCGAAGAGGGAAG
TGCTACACACTTTCAAACAACCAGATCTCGACATGGGCTACTGCCAGGGTGTGAGCCAGGTCGCTGTTGT
CCTGCTGATGTTCCCCAAGGAGAAAGAGGCCTTCTTGGCACTAGCTCAGCTGCTGACCAGCAAAAACCTG
CCAGACACTGTAGATGGACAGCTGCCTATGGGGCCTCACAGCCGGGCCAGCCAGGTGGCTCCAGAGACGA
CATCAAGCAAGGTGGACCGGGGTGTCTCCACAGTGTGTGGGAAGCCTAAGGTGGTGGGGAAGATCTATGG
TGGCCGGGACGCAGCAGCTGGCCAGTGGCCATGGCAGGCCAGCCTGCTCTACTGGGGCTCGCACCTCTGT
GGAGCTGTCCTCATCGACTCCTGCTGGCTGGTATCAACTACCCACTGCTTTAAATCCCAGGCCCCGAAGA
ACTATCAGGTTCTGTTGGGAAACATCCAACTGTATCATCAAACCCAGCACACCCAGAAGATGTCTGTGCA
CCGGATCATCACCCATCCAGACTTTGAGAAGCTCCACCCCTTTGGGAGTGACATTGCCATGTTGCAGCTG
CACCTGCCTATGAACTTCACTTCCTACATTGTCCCTGTCTGCCTCCCATCCCGGGACATGCAGCTGCCCA
GTAACGTGTCCTGTTGGATAACCGGCTGGGGAATGCTCACCGAAGACCTTTGTTCTCAGGGCGATTCTGG
GGGGCCTCTAGTCTGCTACCTCCCCAGTGCCTGGGTCCTGGTGGGGCTGGCCAGCTGGGGCCTGGACTGC
CGGCATCCTGCCTACCCCAGCATCTTCACCAGGGTCACCTACTTCATCAACTGGATTGACAAAATCATGA
GGCTCACTCCTCTTTCTGACCCCGCGCTGGCTCCTCACACCTGCTCTCCACCCAAGCCTCTGAGGGCTGC
TGGCCTGCCTGGGCCCTGCGCAGCCCTTGTGCTGCCACAGACCTGGCTCCTGCTGCCACTTACCCTCAGG
GCCCCATGGCAGACCCTGTGATGACCGCAGAGCCCCTCGACCCCTTCTCTCTGCTCGGCCTAG

The nucleic acid sequence of NOV12 maps to chromosome 9 and has 354 of 536 bases (66%) identical to a gb:GENBANK-ID:AB0089101acc:AB008910.1 mRNA from Mus musculus (Mus musculus mRNA for TESP1, complete cds) (E=10.4e⁻⁴³).

A disclosed NOV12 polypeptide (SEQ ID NO:32) is 356 amino acid residues and is presented using the one letter code in Table 12B. The SignalP, Psort and/or Hydropathy results predict that NOV12 does not have a signal peptide and is likely to be localized to the microbody (peroxisome) with a certainty of 0.5783. In alternative embodiments, a NOV12 polypeptide is located to the lysosome (lumen) with a certainty of 0.2299 or the mitochondrial matrix space with a certainty of 0.1000.

TABLE 12B
NOV12 protein sequence

(SEQ ID NO:32)

MAEGEGEASTSSHGDGREKAKREVLHTFKQPDLDMGYCQGVSQVAVVLLMFPKEKEAFLALAQLLTSKNLPD
TVDGQLPMGPHSRASQVAPETTSSKVDRGVSTVCGKPKVVGKIYGGRDAAAGQWPWQASLLYWGSHLCGAVL
IDSCWLVSTTHCFKSQAPKNYQVLLGNIQLYHQTQHTQKMSVHRIITHPDFEKLHPFGSDIAMLQLHLPMNF
TSYIVPVCLPSRDMQLPSNVSCWITGWGMLTEDLCSQGDSGGPLVCYLPSAWVLVGLASWGLDCRHPAYPSI
FTRVTYFINWIDKIMRLTPLSDPALAPHTCSPPKPLRAAGLPGPCAALVLPQTWLLLPLTLRAPWQTL

The NOV12 amino acid sequence was found to have 140 of 142 amino acid residues (98%) identical to, and 140 of 142 amino acid residues (98%) similar to, the 148 amino acid residue ptnr:TREMBLNEW-ACC:CAC12709 protein from Homo sapiens (Human) (BA62C3.1 (SIMILAR TO TESTICULAR SERINE PROTEASE)) (E=1.4e⁻⁴³). NOV12 is expressed in at least in Testis. Expression information was derived from the tissue sources of the sequences that were included in the derivation of the sequence of NOV12.

NOV12 also has homology to the amino acid sequences shown in the BLASTP data listed in Table 12C.

TABLE 12C
BLAST results for NOV12

| Gene Index/ | Protein/ | Length | Identity | Positives | |
|---|---|---|---|---|---|
| Identifier | Organism | (aa) | (%) | (%) | Expect |
| gi\|17469644\|ref\| | similar to | 365 | 305/372 | 307/372 | e−161 |
| XP_071013.1\|(XM_071013) | bA62C3.1 | | (81%) | (81%) | |
| | (similar to | | | | |
| | testicular | | | | |
| | serine protease) | | | | |
| | [Homo sapiens] | | | | |
| gi\|12314133\|emb\| | bA62C3.1 | 148 | 140/142 | 140/142 | 3e−77 |
| CAC12709.1\|(AL136097) | (similar to | | (98%) | (98%) | |
| | testicular | | | | |
| | serine protease) | | | | |
| | [Homo sapiens] | | | | |
| gi\|6678293\|ref\| | testicular | 367 | 108/287 | 160/287 | 3e−49 |
| NP_033381.1\|(NM_009355) | serine protease | | | (55%) | |
| | 1 [Mus musculus] | | | | |
| gi\|6678295\|ref\| | testicular | 366 | 95/276 | 135/276 | 2e−41 |
| NP_033382.1\|(NM_009356) | serine protease | | (34%) | (48%) | |
| | 2 [Mus musculus] | | | | |
| gi\|6009515\|dbj\| | epidermis | 389 | 86/265 | 123/265 | 1e−37 |
| BAA84941.1\|(AB018694) | specific serine | | (32%) | (45%) | |
| | protease | | | | |
| | [Xenopus laevis] | | | | |

The homology of these sequences is shown graphically in the ClustalW analysis shown in Table 12D.

[image]

[image]

[image]

Tables 12E and 12F list the domain descriptions from DOMAIN analysis results against NOV12. This indicates that the NOV12 sequence has properties similar to those of other proteins known to contain these domains.

TABLE 12E
Domain Analysis of NOV12

gnl|Smart|smart00020, Tryp_SPc, Trypsin-like serine protease; Many of these are
synthesised as inactive precursor zymogens that are cleaved during limited
proteolysis to generate their active forms. A few, however, are active as single
chain molecules, and others are inactive due to substitutions of the catalytic
triad residues.
CD-Length = 230 residues, 100.0% aligned
Score = 174 bits (442), Expect = 6e-45

| | | | | |
|---|---|---|---|---|
| NOV12: | 114 | KIYGGRDAAAGQWPWQASLLY-WGSHLCGAVLIDSCWLVSTTHCFKSQAPKNYQVLLGNI | 172 | (SEQ ID NO:220) |
| | | +\| \|\| +\| \| +\|\|\| \|\| \| \| \| \|\| \|\| \|+++ \|\| \|\| + +\| \|\|+ | | |
| Sbjct: | 1 | RIVGGSEANIGSFPWQVSLQYRGGRHFCGGSLISPRWVLTAAHCVYGSAPSSIRVRLGSH | 60 | (SEQ ID NO:221) |
| NOV12: | 173 | QLYHQTQHTQKMSVHRIITHPDFEKLHPFGSDIAMLQLHLPMNFTSYIVPVCLPSRDMQL | 232 | |
| | | \| + \|\| + \| ++\| \|\|++ + +\|\|\|+\|+\| \|+ + + \|+\|\|\|\| + | | |
| Sbjct: | 61 | DLS-SGEETQTVKVSKVIVHPNYNP-STYDNDIALLKLSEPVTLSDTVRPICLPSSGYNV | 118 | |
| NOV12: | 233 | PSNVSCWITGWG-------------------MLTEDLCS--------------------- | 252 | |
| | | \|+ +\| ++\|\|\| +++ \| | | |
| Sbjct: | 119 | PAGTTCTVSGWGRTSESSGSLPDTLQEVNVPIVSNATCRRAYSGGPAITDNMLCAGGLEG | 178 | |
| NOV12: | 253 | -----QGDSGGPLVCYLPSAWVLVGLASWGLD-CRHPAYPSIFTRVTYFINWI | 299 | |
| | | \|\|\|\|\|\|\|\|\|\| \| \|\|\|\|\|+ \|\|\| \| \| \| ++\|\|\|+ +++\|\| | | |
| Sbjct: | 179 | GKDACQGDSGGPLVCNDPR-WVLVGIVSWGSYGCARPNKPGVYTRVSSYLDWI | 230 | |

TABLE 12F
Domain Analysis of NOV12

gnl|Pfam|pfam00089, trypsin, Trypsin. Proteins recognized include all proteins
in families Si, S2A, S2B, S2C, and SS in the classification of peptidases.
Also included are proteins that are clearly members, but that lack peptidase
activity, such as haptoglobin and protein Z (PRTZ*).
CD-Length = 217 residues, 100.0% aligned
Score = 153 bits (386), Expect = 2e-38

| | | | | |
|---|---|---|---|---|
| NOV12: | 115 | IYGGRDAAAGQWPWQASLLYWGSHLCGAVLIDSCWLVSTTHCFKSQAPKNYQVLLGNIQL | 174 | (SEQ ID NO:222) |
| | | \| \|\|\|+\| \|\| +\|\|\| \|\| \| \|\| \|\| \|+++ \|\| + +\|+\|\| \| | | |
| Sbjct: | 1 | IVGGREAQAGSFPWQVSLQVSSGHFCGGSLISENWVLTAAHCVSG--ASSVRVVLGEHNL | 58 | (SEQ ID NO:223) |
| NOV12: | 175 | YHQTQHTQKMSVHRIITHPDFEKLHPFGSDIAMLQLHLPMNFTSYIVPVCLPSRDMQLPS | 234 | |
| | | \|\| \| +\|\| \|\|++ \| +\|\|\|+\|+\| \|+ + \|+\|\|\|\| \|\| | | |
| Sbjct: | 59 | GTTEGTEQKFDVKKIIVHPNYN---PDTNDIALLKLKSPVTLGDTVRPICLPSASSDLPV | 115 | |
| NOV12: | 235 | NVSCWITGWG-----------------MLTEDLCS-----------------------QG | 254 | |
| | | +\| ++\|\|\| +++ + \| \|\| | | |
| Sbjct: | 116 | GTTCSVSGWGRTKNLGTSDTLQEVVVPIVSRETCRSAYGGTVTDTMICAGALGGKDACQG | 175 | |
| NOV12: | 255 | DSGGPLVCYLPSAWVLVGLASWGLDCRHPAYPSIFTRVTYFINWI | 299 | |
| | | \|\|\|\|\|\|\|\| \| \|\|\|+ \|\|\| \| \|\| ++\|\|\|+ +++\|\| | | |
| Sbjct: | 176 | DSGGPLVC---SDGELVGIVSWGYGCAVGNYPGVYTRVSRYLDWI | 217 | |

Proteolytic enzymes that exploit serine in their catalytic activity are ubiquitous, being found in viruses, bacteria and eukaryotes. They include a wide range of peptidase activity, including exopeptidase, endopeptidase, oligopeptidase and omega-peptidase activity. Over 20 families (denoted S1-S27) of serine protease have been identified, these being grouped into 6 clans (SA, SB, SC, SE, SF and SG) on the basis of structural similarity and other functional evidence. Structures are known for four of the clans (SA, SB, SC and SE): these appear to be totally unrelated, suggesting at least four evolutionary origins of serine peptidases and possibly many more. See Interpro (IPR001254).

Notwithstanding their different evolutionary origins, there are similarities in the reaction mechanisms of several peptidases. Chymotrypsin, subtilisin and carboxypeptidase C clans have a catalytic triad of serine, aspartate and histidine in common: serine acts as a nucleophile, aspartate as an electrophile, and histidine as a base. The geometric orientations of the catalytic residues are similar between families, despite different protein folds. The linear arrangements of the catalytic residues commonly reflect clan relationships. For example the catalytic triad in the chymotrypsin clan (SA) is ordered HDS, but is ordered DHS in the subtilisin clan (SB) and SDH in the carboxypeptidase clan (SC).

The trypsin family is almost totally confined to animals, although trypsin-like enzymes are found in actinomycetes of the genera Streptomyces and Saccharopolyspora, and in the fungus Fusarium oxysporum. The enzymes are inherently secreted, being synthesised with a signal peptide that targets them to the secretory pathway. Animal enzymes are either secreted directly, packaged into vesicles for regulated secretion, or are retained in leukocyte granules.

The protein similarity information, expression pattern, cellular localization, and map location for the NOV12 protein and nucleic acid disclosed herein suggest that this Testicular Serine Protease-like protein may have important structural and/or physiological functions characteristic of the trypsin family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from prostate cancer or infertility as well as other diseases, disorders and conditions.

The novel nucleic acid encoding the Testicular Serine Protease-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV12 protein has multiple hydrophilic regions, each of which can be used as an immunogen. In one embodiment, a contemplated NOV12 epitope is from about amino acids 10 to 25. In another embodiment, a contemplated NOV12 epitope is from about amino acids 70 to 85. In other specific embodiments, contemplated NOV12 epitopes are from about amino acids 101 to 104, 120 to 140, 155 to 205, 240 to 245, 260 to 265, 290 to 298 and 310 to 320.

NOV13

One NOVX protein of the invention, referred to herein as NOV13, includes two Hepatitis B Virus (HBV) Associated Factor-like proteins. The disclosed proteins have been named NOV13a and NOV13b.

NOV13a

A disclosed NOV13a (designated CuraGen Acc. No. CG56961-01), which encodes a novel Hepatitis B (HBV) Associated Factor-like protein and includes the 2393 nucleotide sequence (SEQ ID NO:33) is shown in Table 13A. An open reading frame for the mature protein was identified beginning with an ATG initiation codon at nucleotides 157-159 and ending with a TGA stop codon at nucleotides 1687-1689. Putative untranslated regions are underlined in Table 13A, and the start and stop codons are in bold letters.

TABLE 13A
NOV13a Nucleotide Sequence

(SEQ ID NO:33)

ACAGCATAATATCAAAACACACAGGGCTCGBGCCGCGCCGGAGGCCACACGGCCTGGCTGAGTTGCTCCTGGT
CTCCCGCCTCTCCCAGGCGACCCGGAGGTAGCATTTCCCAGGAGGCACGGTCCCCCCCAGGGGGATGGGCACA
GCCACGCCAGATGGACGAGAAGACCAAGAAAGCAGAGGAAATGGCCCTGAGCCTCACCCGAGCAGTGGCGGGC
GGGATGAACAGGTGGCAATGAAGTGTGCCATCTGGCTGGCAGAGCAACGGGTGCCCCTGAGTGTGCAACTGA
AGCCTGAGGTCTCCCCAACGCAGGACATCAGGCTGTGGGTGAGCGTGGAGGATGCTCAGATGCACACCGTCAC
CATCTGGCTCACAGTGCGCCCTGATATGACCGTGGCGTCTCTCAAGGACATGGTTTTTCTGGACTATGGCTTC
CCACCAGTCTTGCAGCAGTGGGTGATTGGGCAGCGGCTGGCACGAGACCAGGAGACCCTGCACTCCCATGGGG
TGCGGCAGAATGGGGACAGTGCCTACCTCTATCTGCTGTCAGCCCGCAACACCTCCCTCAACCCTCAGGAGCT
GCAGCGGGAGCGGCAGCTGCGGATGCTGGAAGATCTGGGCTTCAAGGACCTCACGCTGCAGCCGCGGGGCCCT
CTGGAGCCAGGCCCCCCAAAGCCCGGGGTCCCCCAGGAACCCGGACGGGGGCAGCCAGATGCAGTGCCTGAGC
CCCCACCGGTGGGCTGGCAGTGCCCCGGGTGCACCTTCATCAACAAGCCCACGCGGCCTGGCTGTGAGATGTG
CTGCCGGGCGCGCCCCGAGGCCTACCAGGTCCCCGCCTCATACCAGCCCGACGAGGACGAGCGAGCGCGCCTG
GCGGGCGAGGAGGAGGCGCTGCGTCAGTACCAGCAGCGGAAGCAGCAGCAGCAGGAGGGGAACTACCTGCAGC
ACGTCCAGCTGGACCAGAGGAGCCTGGTGCTGAACACGGAGCCCGCCGAGTGCCCCGTGTGCTACTCGGTGCT
GGCGCCCGGCGAGGCCGTGGTGCTGCGTGAGTGTCTGCACACCTTCTGCAGGGAGTGCCTGCAGGGCACCATC
CGCAACAGCCAGGAGGCGGAGGTCTCCTGCCCCTTCATTGACAACACCTACTCGTGCTCGGGCAAGCTGCTGG
AGAGGGAGATCAAGGCGCTCCTGACCCCTGAGGATTACCAGCGATTTCTAGACCTGGGCATCTCCATTGCTGA
AAACCGCAGTGCCTTCAGCTACCATTGCAAGACCCCAGATTGCAAGGGATGGTGCTTCTTTGAGGATGATGTC
AATGAGTTCACCTGCCCTGTGTGTTTCCACGTCAACTGCCTGCTCTGCAAGGCCATCCATGAGCAGATGAACT
GCAAGGAGTATCAGGAGGACCTGGGCCTGCCGGCTCAGAACGATGTGGCTGCCCGGCAGACGACAGAGATGCT
GAAGGTGATGCTGCAGCAGGGCGAGGCCATGCGCTGCCCCCAGTGCCAGAGTCGTGGTACAGAAGAAGGACGGC
TGCGACTGGATCCGCTGCACCGTCTGCCACACCGAGATCTGCTGGGTCACCAAGGGCCCACGCTGGGGCCCTG
GGGGCCCAGGAGACACCAGCGGGGGCTGCCGCTGTAGGGTAAATGGGATTCCTTGCCACCCAAGCTGTCAGAA
CTGCCACTGAGCTAAAGATGGTGGGGCCACATGCTGACCCAGCCCCACATCCACATTCTGTTAGAATGTAGCT
CAGGGAGCTTCGTGGACGGCCTTGCTTGCTGTAGCGTTGTAGGGGTCCTGCCTGCACTGCGGTTGTCCACGGT
CACATCTGCCCCAGTGCCTTTGTCCTTCCCTTGGGGCTTGCCGGCCAGACTTCTCTCCCCTGCGGCTCCCACC
TCTGCCTGACCCCAGCCTTAAACATAGCCCCTGGCTAGAGGCCTTGCTGGGTGGAGCCTCTGTGTGACTCCAT
ACTCCTCCCACCACAACACTCATCTGTCAAACACCAAGCACTCTCAGCCTCCCCGCCTTCAGCTGTCAGCTTT
CTGGGGCTAACTTCTCTGCCTTTGTGGTTGGAGGCCTGAGGCCTCTTGGAACTCTTGCTAACCTGTTCAGAGC
CAGGAAGGAGACTGCACAGTTTTGAAAGCACAGCCCGTCAGGTCCGGCTCTGCGTCTCCCTCTCTGCAACCTG
TGTAAGCTATTATAATTAAAATGGTTTTCCGGGAAGGGATGAGTGTGATGTCCTTGAGAGGAAATGAATGCCC
TGGCCTGGGACTCTACACACAGGCAGGATCCTGAGGTCTCTGGGAACTGCATCAGAAAGTTGACTTGTCAGTC
CATCTGTGGTAGAATGAGGCTGTGACTGAGCACTGGGACCTTTCTACCAGATGTGGC

The disclosed NOV13a nucleic acid sequence maps to chromosome 20 and 1894 of 1900 bases (99%) identical to a gb:GENBANK-ID:HSU67322|acc:U67322.1 mRNA from Homo sapiens (Human HBV associated factor (XAP4) mRNA, complete cds) (E=0.0).

A disclosed NOV13a polypeptide (SEQ ID NO:34) is 510 amino acid residues in length and is presented using the one-letter amino acid code in Table 13B. The SignalP, Psort and/or Hydropathy results predict that NOV13a does not have a signal peptide and is likely to be localized to the cytoplasm with a certainty of 0.4500. In alternative embodiments, a NOV13a polypeptide is located to the microbody (peroxisome) with a certainty of 0.3000, the mitochondrial matrix space with a certainty of 0.1000, or in the lysosome (lumen) with a certainty of 0.1000.

TABLE 13B
Encoded NOV13a Protein Sequence

(SEQ ID NO:34)

MDEKTKKAEEMALSLTRAVAGGDEQVAMKCAIWLAEQRPVLSVQLKPEVSPTQDIRLWVSVEDAQMHTVTIWLTV
RPDMTVASLKDMVFLDYGFPPVLQQWVIGQRLARDQETLHSHGVRQNGDSAYLYLLSARNTSLNPQELQRERQLR
MLEDLGFKDLTLQPRGPLEPGPPKPGVPQEPGRGQPDAVPEPPPVGWQCPGCTFINKPTRPGCEMCCRARPEAYQ
VPASYQPDEEERARLAGEEEALRQYQQRKQQQQEGNYLQHVQLDQRSLVLNTEPAECPVCYSVLAPGEAVVLREC
LHTFCRECLQGTIRNSQEAEVSCPFIDNTYSCSGKLLEREIKALLTPEDYQRFLDLGISIAENRSAFSYHCKTPD
CKGWCFFEDDVNEFTCPVCFHVNCLLCKAIHEQMNCKEYQEDLALRAQNDVAARQTTEMLKVMLQQGEAMRCPQC
QIVVQKKDGCDWIRCTVCHTEICWVTKGPRWGPGGPGDTSGGCRCRVNGIPCHPSCQNCH

The NOV13a amino acid sequence was found to have 457 of 464 amino acid residues (98%) identical to, and 459 of 464 amino acid residues (98%) similar to, the 468 amino acid residue ptnr:SPTREMBL-ACC:O95623 protein from Homo sapiens (Human) (HBV ASSOCIATED FACTOR) (E=9.4e⁻²⁶³).

NOV13a is expressed in at least the liver. Expression information was derived from the tissue sources of the sequences that were included in the derivation of the sequence of NOV13a.

Possible small nucleotide polymorphisms (SNPs) found for NOV13a are listed in Tables 13C and 13D.

TABLE 13C
SNPs

| Consensus Position | Depth | Base Change | PAF |
|---|---|---|---|
| 1000 | 9 | T > G | 0.444 |

TABLE 13D
SNPs

| | Nucleotide | | Amino Acid | |
|---|---|---|---|---|
| Variant | Position | Base Change | Position | Base Change |
| 13376998 | 1249 | A > G | 365 | Ser > Gly |

NOV13b

A disclosed NOV13b (designated CuraGen Acc. No. CG56961-02), which includes the 2372 nucleotide sequence (SEQ ID NO:35) shown in Table 13E. An open reading frame for the mature protein was identified beginning with an ATG codon at nucleotides 1-3 and ending with a TGA codon at nucleotides 1666-1668. The start and stop codons of the open reading frame are highlighted in bold type. Putative untranslated regions are underlined.

TABLE 13E
NOV13b Nucleotide Sequence

(SEQ ID NO:35)

ATGGGCTCGGGGCGCGTCGGAGGCCACACGGCTGGCTGAGTTGCTCCTGGTCTCCCGCCTCTCCCAGGCGACC
CGGAGGTAGCATTTCCCAGGAGGCACGGTCCCCCCAGGGGGATGGGCACAGCCACGCCAGATGGACGAGAAGA
CCAAGAAAGCAGAGGAAATGGCCCTGAGCCTCACCCGAGCAGTGGCGGGCGGGGATGAACAGGTGGCAATGAAG
TGTGCCATCTGGCTGGCAGAGCAACGGGTGCCCCCGAGTGTGCAACTGAAGCCTGAGGTCTCCCCAACGCAAGA
CATCAGGCTGTGGGTGAGCGTGGAGGATGCTCAGATGCACACCGTCACCATCTGGCTCACAGTGCGCCCTGATA
TGACCGTGGCGTCTCTCAAGGACATGGTTTTTCTGGACTATGGCTTCCCACCAGTCTTGCAGCAGTGGGTGATT
GGGCAGCGGCTGGCACGAGACCAGGAGACCCTGCACTCCCATGGGGTGCGGCAGAATGGGGACAGTGCCTACCT
CTATCTGCTGTCAGCCCGCAACACCTCCCTCAACCCTCAGGAGCTGCAGCGGGAGCGGCAGCTGCGGATGCTGG
AAGATCTGGGCTTCAAGGACCTCACGCTGCAGCCGCGGGGCCCTCTGGAGCCAGGCCCCCCAAAGCCCGGGGTC
CCCCAGGAACCGGACGGGGGCAGCCAGATGCAGTGCCTGAGCCCCCACCGGTGGGCTGGCAGTGCCCCGGGTG
CACCTTCATCAACAAGCCCACGCGGCCTGGCTGTGAGATGTGCTGCCGGGCGCGCCCCGAGGCCTACCAGGTCC
CCGCCTCATACCAGCCCGACGAGGAGGAGCGAGCGCGCCTGGCGGGCGAGGAGGAGGCGCTGCGTCAGTACCAG
CAGCGGAAGCAGCAGCAGCAGGAGGGGAACTACCTGCAGCACGTCCAGCTGGACCAGAGGAGCCTGGTGCTGAA
CACGGAGCCCGCCGAGTGCCCCGTGTGCTACTCGGTGCTGGCGCCCGGCGAGGCCGTGGTGCTGCGTGAGTGTC
TGCACACCTTCTGCAGGGAGTGCCTGCAGGGCACCATCCGCAACAGCCAGGAGGCGGAGGTCTCCTGCCCCTTC
ATTGACAACACCTACTCGTGCTCGGGCAAGCTGCTGGAGAGGGAGATCAAGGCGCTCCTGACCCCTGAGGATTA
CCAGCGATTTCTAGACCTGGGCATCTCCATTGCTGAAAACCGCAGTGCCTTCAGCTACCATTGCAAGACCCCAG
ATTGCAAGGGATGGTGCTTCTTTGAGGATGATGTCAATGAGTTCACCTGCCCTGTGTGTTTCCACGTCAACTGC
CTGCTCTGCAAGGCCATCCATGAGCAGATGAACTGCAAGGAGTATCAGGAGGACCTGGCCCTGCGGGCTCAGAA
CGATGTGGCTGCCCGGCAGACGACAGAGATGCTGAAGGTGATGCTGCAGCAGGGCGAGGCATGCGCTGCCCCC
AGTGCCAGATCGTGGTACAGAAGAAGGACGGCTGCGACGGGATCCGCTGCACCGTCTGCCACAGGGAGATCTGC
TGGGTCACCAAGGGCCCACGCTGGGGCCCTGGGGGCCCAGGAGACACCAGCGGGGGCTGCCGCTGTAGGGTAAA
TGGGATTCCTTGCCACCCAAGCTGTCAGAACTGCCACTGAGCTAAAGATGGTGGGGCCACATGCTGACCCAGCC
CCACATCCACATTCTGTTAGAATGTAGCTCAGGGAGCTTCGTGGACGGCCTTCGTTGCTTGCTGTAGCGTTGTAGGGG
TCCTGCCTGCACTGCGGTTGTCCACGGTCACATCTGCCCCAGTGCCTTTGTCCTTCCCTTGGGGCTTGCCGGCC
AGACTTCTTCTCCCCTGCGGCTCCCACCTCTGCCTGACCCCAGCCTTAAACATAGCCCCTGGCTAGAGGCCTTGC
TGGGTGGAGCCTCTGTGTGACTCCATACTCCTCCCACCACAACACTCATCTGTCAAACACCAAGCACTCTCAGC
CTCCCCGCCTTCAGCTGTCAGCTTTCTGGGCTAACTTCTCTGCCCTTTGTGGTTGGAGGCCTGAGGCCTCTTGG
AACTCTTGCTAACCTGTTCAGAGCCAGGAAGGAGACTGCACAGTTTTGAAAGCACAGCCCGTCAGGTCCGGCTC
TGCGTCTCCCTCTCTGCAACCTGTGTAAGCTATTATAATTAAAATGGTTTTCCGGGAAGGGATGAGTGTGATGT
CCTTGAGAGGAAATGAATGCCCTGGCCTGGGACTCTACACACAGGCAGGATCCTGAGGTCTCTGGGAACTGCAT
CAGAAAGTTGACTTGTCAGTCCATCTGTGGTAGAATGAGGCTGTGACTGAGCACTGGGACCTTTCTACCAGATG
TGGC

The disclosed NOV13b nucleic acid sequence maps to chromosome 20 and has 1949 of 1993 bases (97%) identical to a gb:GENBANK-ID:HSU67322|acc:U67322.1 mRNA from Homo sapiens (Human HBV associated factor (XAP4) mRNA, complete cds) (E=0.0).

A disclosed NOV13b polypeptide (SEQ ID NO:36) is 555 amino acid residues in length and is presented using the one-letter amino acid code in Table 13F. The SignalP, Psort and/or Hydropathy results predict that NOV13b does not have a signal peptide and is likely to be localized to the cytoplasm with a certainty of 0.4500. In alternative embodiments, a NOV13b polypeptide is located to the microbody (peroxisome) with a certainty of 0.3000, the mitochondrial matrix space with a certainty of 0.1000, or the lysosome (lumen) with a certainty of 0.1000.

TABLE 13F
Encoded NOV13b Protein Sequence

(SEQ ID NO:36)

MGSGRVGGHTAWLSCSWSPASPRRPGGSISQEARSPPGGWAQPRQMDEKTKKAEEMALSLRTAVAGGDEQVAMKC
AIWLAEQRVPPSVQLKPEVSPTQDIRLWVSVEDAQMHTVTIWLTVRPDMTVASLKDMVFLDYGFPPVLQQWVIGQ
RLARDQETLHSHGVRQNGDSAYLYLLSARNTSLNPQELQRERQLRMLEDLGFKDLTLQPRGPLEPGPPKPGVPQE
PGRGQPDAVPEPPPVGWQCPGCTFINKPTRPGCEMCCRARPEAYQVPASYQPDEEERARLAGEEEALRQYQQRKQ
QQQEGNYLQHVQLDQRSLVLNTEPAECPVCYSVLAPGEAVVLRECLHTFCRECLQGTIRNSQEAEVSCPFIDNTY
SCSGKLLEREIKALLTPEDYQRFLDLGISIAENRSAFSYHCKTPDCKGWCFFEDDVNEFTCPVCFHVNCLLCKAI
HEQMNCKEYQEDLLRAQNDVAARQTTEMLKVMLQQGEAMRCPQCQIVVQKKDGCDWIRCTVCHTEICWVTKGPR
WGPGGPGDTSGGCRCRVNGIPCHPSCQNCH

The NOV13b amino acid sequence was found to have 499 of 500 amino acid residues (99%) identical to, and 499 of 500 amino acid residues (99%) similar to, the 500 amino acid residue ptnr:TREMBLNEW-ACC:CAC28312 protein from Homo sapiens (Human) (DJ852M4.1.2 (HBV ASSOCIATED FACTOR (ISOFORM 2))) (E=1.3e⁻²⁸⁵).

NOV13b is expressed in at least the following tissues: adrenal gland, bone marrow, brain—amygdala, brain—cerebellum, brain—hippocampus, brain—substantia nigra, brain—thalamus, brain-whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the sequence of NOV13b.

NOV13a and NOV13b are very closely homologous as is shown in the amino acid alignment in Table 13G.

[image]

[image]

Homologies to any of the above NOV13 proteins will be shared by the other NOV13 proteins insofar as they are homologous to each other as shown above. Any reference to NOV13 is assumed to refer to both of the NOV13 proteins in general, unless otherwise noted.

NOV13a also has homology to the amino acid sequences shown in the BLASTP data listed in Table 13H.

TABLE 13H
BLAST results for NOV13a

| Gene Index/ | | Length | Identity | Positives | |
|---|---|---|---|---|---|
| Identifier | Protein/Organism | (aa) | (%) | (%) | Expect |
| gi\|15929590\|gb\| | HBV associated | 510 | 510/510 | 510/510 | 0.0 |
| AAH15219.1\|AAH15219 | factor [Homo | | (100%) | (100%) | |
| (BC015219) | sapiens] | | | | |
| gi\|14043036\|ref\| | chromosome 20 | 500 | 500/500 | 500/500 | 0.0 |
| NP_112506.1\|(NM_031229) | open reading | | (100%) | (100%) | |
| | frame 18, isoform | | | | |
| | 2; HBV associated | | | | |
| | factor [Homo | | | | |
| | sapiens] | | | | |
| gi\|5454168\|ref\| | chromosome 20 | 468 | 455/455 | 455/455 | 0.0 |
| NP_006453.1\|(NM_006462) | open reading | | (100%) | (100%) | |
| | frame 18, isoform | | | | |
| | 1; HBV associated | | | | |
| | factor [Homo | | | | |
| | sapiens] | | | | |
| gi\|9790279\|ref\| | ubiquitin | 498 | 455/500 | 472/500 | 0.0 |
| NP_062679.1\|(NM_019705) | conjugating | | (91%) | (94%) | |
| | enzyme 7 | | | | |
| | interacting | | | | |
| | protein 3 [Mus | | | | |
| | musculus] | | | | |
| gi\|11120718\|ref\| | protein kinase C- | 498 | 453/500 | 474/500 | 0.0 |
| NP_068532.1\|(NM_021764) | binding protein | | (90%) | (94%) | |
| | Beta15 [Rattus | | | | |
| | norvegicus] | | | | |

The homology of these sequences is shown graphically in the ClustalW analysis shown in Table 13I.

[image]

[image]

Tables 13J-K lists the domain description from DOMAIN analysis results against NOV13. This indicates that the NOV13 sequence has properties similar to those of other proteins known to contain these domains, including the gnl|Load|LOAD_little_fing, little_fing, Zinc coordinating RNA binding domain.

TABLE 13J
Domain Analysis of NOV13

HMM file: pfamHMMs
Scores for sequence family classification (score includes all domains):

| | | | | |
|---|---|---|---|---|
| Model | Description | Score | E-value | N |
| -------- | ----------- | ----- | ------- | --- |
| zf-RanBP | Zn-finger in Ran bind prot & others. | 24.3 | 0.0028 | 1 |
| zf-C3HC4 | Zinc finger, C3HC4 type (RING finger) | 22.3 | 1.5e−05 | 2 |
| IBR | IBR domain | −19.1 | 8.3 | 1 |

Parsed for domains:

| | | | | | | | |
|---|---|---|---|---|---|---|---|
| Model | Domain | seq | seq | hmm | hmm | score | E-value |
| | | from | to | from | to | | |
| -------- | ------- | ----- | ----- | ----- | ----- | ----- | ------- |
| zf-RanBP | 1/1 | 194 | 222 .. | 1 | 32 [ ] | 24.3 | 0.0028 |
| zf-C3HC4 | 1/2 | 282 | 325 .. | 1 | 53 [ . | 26.7 | 6.3e−07 |
| zf-C3HC4 | 2/2 | 387 | 394 .. | 46 | 54 . ] | 0.7 | 63 |
| IBR | 1/1 | 351 | 411 .. | 1 | 72 [ ] | −19.1 | 8.3 |

Alignments of top-scoring domains:
zf-RanBP: domain 1 of 1, from 194 to 222: score 24.3, E = 0.0028

| | | | | |
|---|---|---|---|---|
| | | *−>ragsdWdCissClvqNfatskCvaCqapkps<−* | | (SEQ ID NO:229) |
| | | ++\| \|+\| + \|++ \|++\|+ \| +\| ++\| | | |
| NOV13 | 194 | PVG--WQC-PGCTFINKPTRPGCEMCCRARPE | 222 | (SEQ ID NO:230) |

zf-C3HC4: domain 1 of 2, fron 282 to 325: score 26.7, E = 6.3e−07

| | | | | |
|---|---|---|---|---|
| | | *−>CpICltTFdldepkpfkepvllpCgHsFCskCivellrlsqnsknnsvvkCPl<−* | | (SEQ ID NO:231) |
| | | \|\|+\| ++\| ++ \|\| +\|+\|+\|\|+ \|++ +\|+\|\|+ + \| \|\|+ | | |
| NOV13 | 282 | CPVC-----YSVLAPGEAVVLRECLHTFCRECLQGTIRNSQEAE---VS-CPF | 325 | (SEQ ID NO:232) |

zf-C3HC4: domain 2 of 2, from 387 to 394: score 0.7, E = 63

| | | | | |
|---|---|---|---|---|
| | | *−>nsvykCplC<−* | | (SEQ ID NO:233) |
| | | \| ++ \|\|+\| | | |
| NOV13 | 387 | NEFT-CPVC | 394 | (SEQ ID NO:234) |

IBR: domain 1 of 1, from 351 to 411: score −19.1, E = 8.3

| | | | | |
|---|---|---|---|---|
| | | eKYekfmvrsyveknpdlkwCPgpdCsyavrltevssstelaepprVeCkkPaCgtsFCfkCgaeWHapvsC | | (SEQ ID NO:235) |
| | | +++ ++ + + ++ + + \| \|\|\|+ + + + + + +\|++ \|+++ \| \|+\| \|+ ++\| | | |
| NOV | 351 | QRFLDLGISIAENRSAFSYHCKTPDCKGWCFFED--------DVNEF TCPV--CFHVNCLLCKAI-HEQMNC | 411 | (SEQ ID NO:236) |

TABLE 13K
Domain Analysis of NOV13

gnl|Smart|smart00213, UBQ, Ubiquitin homologies; Ubiquitin-mediated
proteolysis is involved in the regulated turnover of proteins required for
controlling cell cycle progression
CD-Length = 72 residues, 83.3% aligned
score = 36.2 bits (82), Expected = 0.005

| | | | | |
|---|---|---|---|---|
| NOV13: | 70 | TIWLTVRPDMTVASLKDMVFLDYGFPPVLQQWVI--GRQLARDQETLHSHGVRQNGDSAY | 127 | (SEQ ID NO:237) |
| | | \| \| \|+\| \|\|+ \|\|+ + \| \|\| \|\| +\| \|+ \| \| \|\| +\|+ \|+\| + + | | |
| Sbjct: | 12 | TITLEVKPSDTVSELKEKIADLEGIPPE-QQRLIYKGKVL-EDDRTLAEYGI-QDGSTIH | 68 | (SEQ ID NO:238) |
| NOV13: | 128 | LYL 130 | | |
| | | \| \| | | |
| Sbjct: | 69 | LVL 71 | | |

Ran binding-proteins (RanBPs) are putative nuclear-export terminators, and importin-beta-like molecules, they are known to bind RanGTP and RanGDP. The RanBP zinc finger found mainly in these proteins bind exclusively RanGDP (Blobel G., Yaseen N. R., 1999, Proc. Natl. Acad. Sci. U.S.A. 96: 5516-5521).

The RING-finger is a specialized type of Zn-finger of 40 to 60 residues that binds two atoms of zinc, and is probably involved in mediating protein-protein interactions. There are two different variants, the C3HC4-type and a C3H2C3-type, which is clearly related despite the different cysteine/histidine pattern. The latter type is sometimes referred to as ‘RING-H2 finger’.

E3 ubiquitin-protein ligase activity is intrinsic to the RING domain of c-Cbl and is likely to be a general function of this domain; Various RING fingers exhibit binding to E2 ubiquitin-conjugating enzymes (Ubc's). Several 3D-structures for RING-fingers are known [2, 3]. The 3D structure of the zinc ligation system is unique to the RING domain and is referred to as the ‘cross-brace’ motif. The spacing of the cysteines in such a domain is C-x(2)-C-x(9 to 39)-C-x(1 to 3)—H-x(2 to 3)-C-x(2)-C-x(4 to 48)-C-x(2)-C. The way the ‘cross-brace’ motif is binding two atoms of zinc is illustrated in the following schematic representation:

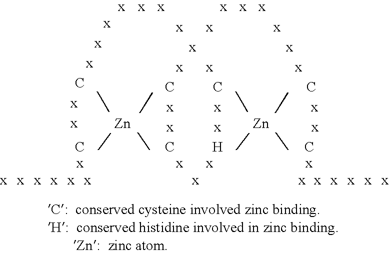

Note that in the older literature, some RING-fingers are denoted as LIM-domains. The LIM-domain Zn-finger is a fundamentally different family, albeit with similar Cys-spacing (see INTERPRO IPR001781, Freemont, 1993, Ann. N.Y. Acad. Sci. 684: 174-192; Freemont and Borden, 1996, Curr. Opin. Struct. Biol. 6: 395-401; Freemont et al., 1996, Trends Biochem. Sci. 21: 208-214; Freemont, 2000, Curr. Biol. volume:10 issue:2; Hunter et al., 1999, Science 286: 309-312; Barinaga, 1999, Science first page:223 volume:286 issue:5438).

Primary cancer of the liver in three brothers was described by Kaplan and Cole (1965) and by Hagstrom and Baker (1968). In these patients there was no recognized preexisting liver disease. Denison et al. (1971) described two adult brothers who died of primary hepatocellular carcinoma. Both had micronodular cirrhosis with features of subacute progressive viral hepatitis. Australia antigen was demonstrated in the brother in whom it was sought. Their father had died much earlier of hepatocellular carcinoma. Familial LCC might also have its explanation in alpha-1-antitrypsin deficiency, hemochromatosis, and tyrosinemia. Integration of the hepatitis B virus (HBV) into cellular DNA occurs during long-term persistent infection in man. Hepatocellular carcinomas isolated from carriers of virus often contain clonally propagated viral DNA. Shen et al. (1991) presented evidence for the interaction of inherited susceptibility and hepatitis B viral infection in cases of primary hepatocellular carcinoma in eastern China. Complex segregation analysis of 490 extended families supported the existence of a recessive allele with population frequency approximately 0.25, which results in a lifetime risk of HCC in the presence of both HBV infection and genetic susceptibility, of 0.84 for males and 0.46 for females. The model further predicted that, in the absence of genetic susceptibility, lifetime risk of HCC is 0.09 for HBV-infected males and 0.01 for HBV-infected females and that regardless of genotype the risk is virtually zero for uninfected persons.

The finding of small deletions in retinoblastoma and Wilms tumor prompted Rogler et al. (1985) to look for the same in association with HBV integration in hepatocellular carcinoma. They demonstrated a deletion of at least 13.5 kb of cellular sequences in a liver cancer. The HBV integration and the deletion occurred on the short arm of chromosome 11 at location 11p14-p13. The deleted sequences were lost in tumor cells leaving only a single copy. Clones of the DNA flanking the deleted segment were used for the mapping of the deletion in somatic cell hybrids and by in situ hybridization. Cellular sequences homologous to the deleted region were cloned and used to exclude the possibility that this DNA had been moved to other positions in the genome. Fisher et al. (1987) extended the observations of Rogler et al. (1985). Using somatic cell hybrids that contained defined 11p deletions, 2 cloned DNA sequences that flank the deletion generated by a hepatocellular carcinoma (as a consequence of hepatitis B virus integration) were mapped to 11p13. Wilms tumor and the tumors of Beckwith-Wiedemann syndrome are also determined by changes on 11p.

Henderson et al. (1988) found that unique cellular DNA to the left of an HBY DNA integration site cloned from a primary tumor mapped to chromosome 18q (18q 11.1-q11.2), whereas right-hand flanking DNA mapped to chromosome 17 at a subterminal region of the long arm. In a hepatoma specimen from Shanghai, Zhou et al. (1988) identified integration of hepatitis B virus into 17p12-p11.2, which is near the human protooncogene p53. Furthermore, the sequence of flanking cellular DNA showed highly significant homology with a conserved region of a number of functional mammalian DNAs, including the human autonomously replicated sequence-1 (ARS1). ARS1 is a sequence of human DNA that allows replication of Saccharomyces cerevisiae integrative plasmids as autonomously replicating elements in S. cerevisiae cells. Since integration of viral DNA is not a required step in the replicative cycle of the hepatitis virus, the presence of integrated HBV sequences in many human hepatocellular carcinomas suggests a causal relationship. Since any one of several integration sites may lead to the same result, the crucial cellular targets involved in triggering liver cell malignant transformation may differ from tumor to tumor. Smith et al. (1989) gave evidence for microdeletions of chromosome 4q involving the alcohol dehydrogenase isoenzyme gene ADH3 and hepatomas from 3 of 5 individuals heterozygous for an XbaI RFLP detectable by the ADH probe. Two of 7 individuals heterozygous for an epidermal growth factor RFLP had lost 1 EGF allele in their hepatoma tissue.

Agarwal et al. (1998) reported a case of severe gynecomastia in a seventeen and one-half-year-old boy due to high levels of aromatase expression in a large fibrolamellar hepatocellular carcinoma, which caused extremely elevated serum levels of estrone (1200 pg/mL) and estradiol-17 (312 pg/mL) that suppressed follicle-stimulating hormone (FSH) and luteinizing hormone (LH) (1.3 and 2.8 IU/L, respectively) and consequently testosterone (1.53 ng/mL). After removal of the 1.5-kg tumor, gynecomastia partially regressed, and normal hormone levels were restored. By immunohistochemistry, diffuse intracytoplasmic aromatase expression was detected in the liver cancer cells. Northern blot analysis showed P450 aromatase transcripts in total RNA from the hepatocellular cancer but not in the adjacent liver nor in disease-free adult liver samples. Promoters I.3 and II were used for P450 aromatase transcription in the cancer.

Primary hepatocellular carcinoma occurs at high frequencies in east Asia and sub-Saharan Africa. In these areas of the world, chronic infection with the hepatitis B virus is the best documented risk factor; however, only 20 to 25% of HBV carriers develop HCC. Exposure to the fungal toxin aflatoxin B1 (AFB1) has been suggested to increase HCC risk, in part because in vitro experiments demonstrated that AFB1 mutagenic metabolites bind to DNA and are capable of inducing G-to-T transversions. In certain areas of the HCC endemic regions, a mutational hotspot has been reported in the p53 tumor suppressor gene (TP53): an AGG-to-AGT transversion (arginine to serine) of codon 249 in exon 7. Microsomal epoxide hydrolase (EPHX) and glutathione-S-transferase M1 (GSTM1) are both involved in AFB1 detoxification in hepatocytes. Polymorphism of both genes has been identified. In Ghana and China, McGlynn et al. (1995) conducted studies to determine whether mutant alleles at one or both of these loci are associated with increased levels of serum AFB1-albumin adducts, with HCC, and with mutations at codon 249 of p53. In a cross-sectional study, they found that mutant alleles at both loci were significantly over-represented in individuals with serum AFB1 albumin adducts. Additionally, in a case-control study, mutant alleles of EPHX were significantly over-represented in persons with HCC. The relationship of EPHX to HCC varied by hepatitis B surface antigen status, indicating that a synergistic effect may exist. Mutations at codon 249 of p53 were observed only among HCC patients with one or both high-risk genotypes. These findings by McGlynn et al. (1995) supported the existence of genetic susceptibility in humans to the environmental carcinogen AFB1 and indicated that there is a synergistic increase in risk of HCC with the combination of hepatitis B virus infection and susceptible genotype.

Schwienbacher et al. (2000) analyzed DNA and RNA from 52 human hepatocarcinoma samples and found abnormal imprinting of genes located at 11p15 in 51% of 37 informative samples. The most frequently detected abnormality was gain of imprinting, which led to loss of expression of genes present on the maternal chromosome. As compared with matched normal liver tissue, hepatocellular carcinoma showed extinction or significant reduction of expression of one of the alleles of the CDKNI C, SLC22A1L, and IGF2 genes. Loss of maternal-specific methylation of the KvDMR1 gene in hepatocarcinoma correlated with abnormal expression of CDKN1C and IGF2, suggesting a function for KvDMR1 as a long-range imprinting center active in adult tissues. These results pointed to the role of epigenetic mechanisms leading to loss of expression of imprinted genes at 11 p15 in human tumors.

See: Agarwal, et al., J. Clin. Endocr. Metab. 83: 1797-1800, 1998. PubMed ID: 9589695; Chang, et al., Cancer 53: 1807-1810, 1984. PubMed ID: 6321015; Denison, et al., Ann. Intern. Med. 74: 391-394, 1971. PubMed ID: 4324021; Fisher, et al., Hum. Genet. 75: 66-69, 1987. PubMed ID: 3026949; Hagstrom and Baker, Cancer 22: 142-150, 1968. PubMed ID: 4298178; Henderson, et al., Cancer Genet. Cytogenet. 30: 269-275, 1988. PubMed ID: 2830013; Kaplan, and Cole, Am. J. Med. 39: 305-311, 1965; Lynch, et al., Cancer Genet. Cytogenet. 11: 11-18, 1984. PubMed ID: 6317164; McGlynn, et al., Proc. Nat. Acad. Sci. 92: 2384-2387, 1995. PubMed ID: 7892276; Rogler, et al., Science 230: 319-322, 1985. PubMed ID: 2996131; Schwienbacher, et al., Proc. Nat. Acad. Sci. 97: 5445-5449, 2000. PubMed ID: 10779553; Shen, et al., Am. J. Hum. Genet. 49: 88-93, 1991. PubMed ID: 1648308; Smith, et al., (Abstract) Cytogenet. Cell Genet. 51: 1081 only, 1989; and Zhou, et al., J. Virol. 62: 42244231, 1988. PubMed ID: 2845134.

The protein similarity information, expression pattern, cellular localization, and map location for the NOV13 protein and nucleic acid disclosed herein suggest that this HBV Associated Factor-like protein may have important structural and/or physiological functions characteristic of the intracellular family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV13 nucleic acids and proteins of the invention have applications in the diagnosis and/or treatment of various diseases and disorders. For example, the compositions of the present invention will have efficacy for the treatment of patients suffering from: Von Hippel-Lindau (VHL) syndrome, cirrhosis, transplantation, cancer, hepatitis B as well as other diseases, disorders and conditions.

The novel nucleic acid encoding the HBV Associate Factor-like protein of the invention, or fragments thereof, are useful in diagnostic applications, wherein the presence or amount of the nucleic acid or the protein are to be assessed.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV13 protein has multiple hydrophilic regions, each of which can be used as an immunogen. In one embodiment, a contemplated NOV13 epitope is from about amino acids 2 to 3. In another embodiment, a contemplated NOV13 epitope is from about amino acids 60 to 70. In other specific embodiments, contemplated NOV13 epitopes are from about amino acids 90 to 92, 110 to 120, 125 to 130, 180 to 195, 200 to 300, 310 to 390, 400 to 410 and 420 to 490.

NOV14

One NOVX protein of the invention, referred to herein as NOV14, includes two Apolipoprotein L-like proteins. The disclosed proteins have been named NOV14a and NOV14b.

NOV14a

A disclosed NOV14a (designated CuraGen Acc. No. CG57104-01), which encodes a novel Apolipoprotein L-like protein and includes the 1233 nucleotide sequence (SEQ ID NO:37) is shown in Table 14A. An open reading frame for the mature protein was identified beginning with an ATG initiation codon at nucleotides 10-12 and ending with a TGA stop codon at nucleotides 1213-1215. Putative untranslated regions are underlined in Table 14A, and the start and stop codons are in bold letters.

TABLE 14A
NOV14a Nucleotide Sequence

(SEQ ID NO:37)

AGACGTGGGATGCACACAGCTCAGAACAGTTGGATCTTGCTCAGTCTCTGTCAGAGGAAGATCCCTTGGA
CAAGAGGACCCTGCCTTGGTGTGAGAGTGAGGGAAGAGGAAGCTGGAACGAGGGTTAAGGAAAACCTTCC
AGTCTGGACAGTGACTGGAGAGCTCCAAGGAAAGCCCCTCGGTAACCCAGCCGCTGGCACCATGAACCCA
GAGAGCAGTATCTTTATTGAGGATTACCTTAAGTATTTCCAGGACCAAGTGAGCAGAGAGAATCTGCTAC
AACTGCTGACTGATGATGAAGCCTGGAATGGATTCGTGGCTGCTGCTGAACTGCCCAGGGATGAGGCAGA
TGAGCTCCGTAAAGCTCTGAACAAGCTTGCAAGTCACATGGTCATGAAGGACAAAAACCGCCACGATAAA
GACCAGCAGCACAGGCAGTGGTTTTTGAAAGAGTTTCCTCGGTTGAAAAGGGAGCTTGAGGATCACATAA
GGAAGCTCCGTGCCCTTGCAGAGGAGGTTGAGCAGGTCCACAGAGGCACCACCATTGCCAATGTGGTGTC
CAACTCTGTTGGCACTACCTCTGGCATTCTGACCCTCCTCGGCCTGGGTCTGGCACCCTTCACAGAAGGA
ATCAGTTTTGTGCTCTTGGACACTGGCATGGGTCTGGGAGCAGCAGCTGCTGTGGCTGGGATTACCTGCA
GTGTGGTAGAACTAGTAAACAAATTGCGGGCACGAGCCCAAGCCCGCAACTTGGACCAAAGCGGCACCAA
TGTAGCAAAGGTGATGAAGGAGTTTGTGGGTGGGAACACACCCAATGTTCTTACCTTAGTTGACAATTGG
TACCAAGTCACACAAGGGATTGGGAGGAACATCCGTGCCATCAGACGAGCCAGAGCCAACCCTCAGTTAG
GAGCGTATGCCCCACCCCCGCATGTCATTGGGCGAATCTCAGCTGAAGGCGGTGAACAGGTTGAGAGGGT
TGTTGAAGGCCCCGCCCAGGCAATGAGCAGAGGAACCATGATCGTGGGTGCAGCCACTGGAGGCATCTTG
CTTCTGCTGGATGTGGTCAGCCTTGCATATGAGTCAAAGCACTTGCTTGAGGGGGCAAAGTCAGAGTCAG
CTGAGGAGCTGAAGAAGCGGGCTCAGCAGCTGGAGGGGAAGCTCAACTTTCTCACCAAGATCCATCAGAT
GCTGCAGCCAGGCCAAGACCAATGACCCCAGAGCAGTGCAGCC

The disclosed NOV14a nucleic acid sequence maps to chromosome 22q12 and has 949 of 1167 bases (81%) identical to a gb:GENBANK-ID:AFO 19225|acc:AF019225.1 mRNA from Homo sapiens (Homo sapiens apolipoprotein L mRNA, complete cds) (E=1.2e⁻¹⁶¹).

A disclosed NOV14a polypeptide (SEQ ID NO:38) is 401 amino acid residues in length and is presented using the one-letter amino acid code in Table 14B. The SignalP, Psort and/or Hydropathy results predict that NOV14a has a signal peptide and is likely to be localized to the endoplasmic reticulum (membrane) with a certainty of 0.6850. In alternative embodiments, a NOV14a polypeptide is located to the plasma membrane with a certainty of 0.6400, the Golgi body with a certainty of 0.4600, or the endoplasmic reticulum (lumen) with a certainty of 0.1000. The SignalP predicts a likely cleavage site for a NOV14a peptide between amino acid positions 16 and 17, i.e. at the sequence CQR-KI.

TABLE 14B
Encoded NOV14a Protein Sequence

(SEQ ID NO:38)

MHTAQNSWILLSLCQRKIPWTRGPCLGVRVREEEAGTRVXTPVWTTGELQGKPLGNPGTPESSIFIEDT
KYFQDQVSRLLQLLTDDEANGFVAAAELPRDEADELRKLNKLASVMKDKQQHRQWFPRI
ELEDHIRXLRALAEEVEQV1RGTTIVVSNSVGTTSGILTLLGLGLAPFTEGISELGMGLGVAGITCS
VVELVLRARAQALDQSGTVAVMKEFVCGNTPNVLTLVDNWYQVTQGIGRNIRAIRNPQLGAYPPH
VIGRISAEGGEQVERVEGPAQAMSRGTMIVGAATCGILLLLDVVSLAYESKHLLEGSESELKQELEG
NFLTKIHEMLQPGQDQ

The NOV14a amino acid sequence was found to have 235 of 377 amino acid residues (62%) identical to, and 284 of 377 amino acid residues (75%) similar to, the 383 amino acid residue ptnr:TREMBLNEW-ACC:AAB81218 protein from Homo sapiens (Human) (APOLIPOPROTEIN L-I) (E 4.6e⁻¹¹²)

NOV14a is expressed in at least the following tissues: adrenal gland, bone marrow, brain—amygdala, brain—cerebellum, brain—hippocampus, brain—substantia nigra, brain—thalamus, brain-whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the sequence of NOV14a. The sequence is predicted to be expressed in the following tissues because of the expression pattern of (GENBANK-ID: gb:GENBANK-ID:AF019225|acc:AF019225.1) a closely related Homo sapiens apolipoprotein L mRNA, complete cds homolog in species Homo sapiens:pancreas. Possible small nucleotide polymorphisms (SNPs) found for NOV14a are listed in Table 14C.

TABLE 14D
SNPs

| | Nucleotide | | Amino Acid | |
|---|---|---|---|---|
| Variant | Position | Base Change | Position | Base Change |
| 13376999 | 746 | C > T | 246 | Arg > Cys |

NOV14b

A disclosed NOV14b (designated CuraGen Acc. No. CG57104-02), which includes the 1232 nucleotide sequence (SEQ ID NO:39) shown in Table 14D. An open reading frame for the mature protein was identified beginning with an ATG codon at nucleotides 9-11 and ending with a TGA codon at nucleotides 1212-1214. The start and stop codons of the open reading frame are highlighted in bold type. Putative untranslated regions are underlined.

TABLE 14D
NOV14b Nucleotide Sequence

(SEQ ID NO:39)
GACGTGGGATGCACATAGCTCAGAACAGTTGGATCTTGCTCAGTCTCTGTCAGAGGAAGATCCCTTGGACAAGAG
GACCCTGCCTTGGTGTGAGAGTGAGGGAAGAGGAAGCTGGAACGAGGGTTAAGGAAAACCTTCCAGTCTGGACAG
TGACTGGAGAGCTCCAAGGAAAGCCCCTCGGTAACCCAGCCGCTGGCACCATGAACCCAGAGAGCAGTATCTTTA
TTGAGGATTACCTTAAGTATTTCCAGGACCAAGTGAGCAGAGAGAATCTGCTACAACTGCTGACTGATGATGAAG
CCTGGAATGGATTCGTGGCTGCTGCTGAACTGCCCAGGGATGAGGCAGATGAGCTCCGTAAAGCTCTGAACAAGC
TTGCAAGTCACATGGTCATGAAGGACAAAAACCGCCACGATAAAGACCAGCAGCACAGGCAGTGGTTTTTGAAAG
AGTTTCCTCGGTTGAAAAGGGAGCTTGAGGATCACATAAGGAAGCTCCGTGCCCTTGCAGAGGAGGTTGAGCAGG
TCCACAGAGGCACCACCATTGCCAATGTGGTGTCCAACTCTGTTGGCACTACCTCTGGCATCCTGACCCTCCTCG
GCCTGGGTCTGGCACCCTTCACAGAAGGAATCAGTTTTGTGCTCTTGGACACTGGCATGGGTCTGGGAGCAGCAG
CTGCTGTGGCTGGGATTACCTGCAGTGTGGTAGAACTAGTAAACAAATTGCGGGCACGAGCCCAAGCCCGCAACT
TGGACCAAAGCGGCACCAATGTAGCAAAGGTGATGAAGGAGTTTGTGGGTGGGAACACACCCAATGTTCTTACCT
TAGTTGACAATTGGTACCAAGTCACACAAGGGATTGGGAGGAACATCCGTGCCATCAGACGAGCCAGAGCCAACC
CTCAGTTAGGAGCGTATGCCCCACCCCCGCATGTCATTGGGCGAATCTCAGCTGAAGGCGGTGAACAGGTTGAGA
GGGTTGTTGAAGGCCCCGCCCAGGCAATGAGCAGAGGAACCATGATCGTGGGTGCAGCCACTGGAGGCATCTTGC
TTCTGCTGGATGTGGTCAGCCTTGCATATGAGTCAAAGCACTTGCTTGAGGGGGCAAAGTCAGAGTCAGCTGAGG
AGCTGAAGAAGCGGGCTCAGGAGCTGGAGGGGAAGCTCAACTTTCTCACCAAGATCCATGAGATGCTGCAGCCAG
GCCAAGACCAATGACCCCAGAGCAGTGCAGCC

The disclosed NOV14b nucleic acid sequence maps to chromosome 22q12 and has 975 of 1200 bases (81%) identical to a gb:GENBANK-ID:AF019225|acc:AF019225.2 mRNA from Homo sapiens (Homo sapiens apolipoprotein L-1 mRNA, complete cds) (E=136e⁻¹⁷⁵).

A disclosed NOV14b polypeptide (SEQ ID NO:40) is 401 amino acid residues in length and is presented using the one-letter amino acid code in Table 14E. The SignalP, Psort and/or Hydropathy results predict that NOV14b has a signal peptide and is likely to be localized to the endoplasmic reticulum (membrane) with a certainty of 0.6850. In alternative embodiments, a NOV14b polypeptide is located to the plasma membrane with a certainty of 0.6400, the Golgi body with a certainty of 0.4600, or the endoplasmic reticulum (lumen) with a certainty of 0.1000. The SignalP predicts a likely cleavage site for a NOV14b peptide between amino acid positions 14 and 15, ie. at the sequence SLC-QR.

TABLE 14E
Encoded NOV14b Protein Sequence

(SEQ ID NO:40)

MIIAQNSWILLSLCQRKIPWTRGPCLGVRVREEEAGTRVKLPVWVTGELQGKPLGNPGThNPESSIIEDY
KRELEDHIRKLRALAEEVEQVHRGTTINVVSNSVGTTSGILTLLGLGLAPFTEGISFVLLDTGMGLGVAGI
TCSVVELVNKLRARAQARNLDQSGTVACVKEFVGGTPNLTLVDNWYQQGIGIIRNPQLGAYA
PPPHVIGRISAEGGEQVERVVEGPAQMSRGTMIVGAATGGILLVVSLAYESKLEGSESELRAQE
LEGKLNFLTKIHEMLQPGQDQ

The NOV14b amino acid sequence was found to have 336 of 337 amino acid residues (99%) identical to, and 337 of 337 amino acid residues (100%) similar to, the 337 amino acid residue ptnr:SWISSNEW-ACC:Q9BQE5 protein from Homo sapiens (Human) (Apolipoprotein L2 (Apolipoprotein L-II) (ApoL-II)) (E=1.3e⁻¹⁷⁴).

NOV14b is expressed in at least the following tissues: adrenal gland, bone marrow, brain—amygdala, brain—cerebellum, brain—hippocampus, brain—substantia nigra, brain—thalamus, brain-whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the sequence of NOV14b. The sequence is predicted to be expressed in the following tissues because of the expression pattern of (GENBANK-ID: gb:GENBANK-ID:AF019225|acc:AF019225.2) a closely related Homo sapiens apolipoprotein L-I mRNA, complete cds homolog in species Homo sapiens:pancreas.

NOV14a and NOV14b are very closely homologous as is shown in the amino acid alignment in Table 14F.

[image]

[image]

Homologies to any of the above NOV14 proteins will be shared by the other NOV14 proteins insofar as they are homologous to each other as shown above. Any reference to NOV14 is assumed to refer to both of the NOV14 proteins in general, unless otherwise noted.

NOV14a also has homology to the amino acid sequences shown in the BLASTP data listed in Table 14G.

TABLE 14G
BLAST results for NOV14a

| Gene Index/ | Protein/ | Length | Identity | Positives | |
|---|---|---|---|---|---|
| Identifier | Organism | (aa) | (%) | (%) | Expect |
| gi\|13325156\| | Similar to | 337 | 337/337 | 337/337 | e−167 |
| gb\|AAH04395.1\| | apolipoprotein L | | (100%) | (100%) | |
| AAH04395 (BC004395) | [Homo sapiens] | | | | |
| gi\|13562090\| | apolipoprotein | 337 | 336/337 | 337/337 | e−167 |
| ref\|NP_112092.1\| | L, 2 [Homo | | (99%) | (99%) | |
| (NM_030882) | sapiens] | | | | |
| gi\|5725224\| | (apolipoprotein | 279 | 278/279 | 279/279 | e−131 |
| emb\|CAB52401.1\| | L, 2) [Homo | | (99%) | (99%) | |
| (Z95114) bK212A2.2 | sapiens] | | | | |
| gi\|12408013\| | apolipoprotein | 414 | 236/383 | 285/383 | e−115 |
| gb\|AAG53690.1\| | L-I [Homo | | (61%) | (73%) | |
| AF323540_1 (AF323540) | sapiens] | | | | |
| gi\|15824471\| | apolipoprotein | 398 | 237/383 | 285/383 | e−115 |
| gb\|AAL09358.1\| | L1 precursor | | (61%) | (73%) | |
| AF305428_1\|(AF305428) | [Homo sapiens] | | | | |

The homology of these sequences is shown graphically in the ClustalW analysis shown in Table 14H.

[image]

[image]

The protein similarity information, expression pattern, cellular localization, and map location for the NOV14 protein and nucleic acid disclosed herein suggest that this Apolipoprotein L-like protein may have important structural and/or physiological functions characteristic of the Apolipoprotein family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

Epidemiological studies have demonstrated a strong inverse correlation between the levels of plasma high density lipoproteins (HDL) and risk of premature coronary heart disease (Miller, G. J., and Miller, N. E., 1975, Lancet 1,16-19, Gordon, et al., 1977, J. Am. Med. Assoc. 238, 497499). However, the mechanisms by which HDL protect against atherosclerosis need further exploration. One proposed protective role of HDL involves reverse cholesterol transport, a process in which HDL acquire cholesterol from peripheral cells and facilitate its esterification and delivery to the liver. In this process, small, relatively lipid-poor HDL particles, termed pre-1-HDL, have been postulated to be the first acceptors of cholesterol from the cells. An additional mechanism may involve the ability of HDL to impede the oxidation of other plasma lipoproteins (Glomset, J. A., 1968, J. Lipid Res. 9, 155-167; Kunitake, et al., 1987, National Institutes of Health Workshop on Lipoprotein Heterogeneity, NIH Publication 87, Vol. 2646, pp. 419-427, National Institutes of Health, Rockville, Md.; Fielding, C. J., and Fielding, P. E. (I 995) J. Lipid Res. 36, 211-228; Castro, G. R., and Fielding, C. J. (1988) Biochemistry 27, 25-29; Francone, et al., 1989, J. Biol. Chem. 264, 7066-7072; Parthasarathy, et al., 1990, Biochim. Biophys. Acta 1044, 275-283; Kunitake et al., 1992, Proc. Natl. Acad. Sci. U.S.A. 89, 6993-6997; Ohta, T., Takata, K., Horiuchi, S., Morino, Y., and Matsuda, I., 1989, FEBS Lett. 257, 435-438).

Recently, Duchateau et al. (1997, J Biol Chem 272: 25576-82) identified and characterized a new protein present in human high density lipoprotein, apolipoprotein L. Expression of apolipoprotein L was only detected in the pancreas. The cDNA sequence encoding the full-length protein was cloned using reverse transcription-polymerase chain reaction. The deduced amino acid sequence contains 383 residues, including a typical signal peptide of 12 amino acids. No significant homology was found with known sequences. The plasma protein is a single chain polypeptide with an apparent molecular mass of 42 kDa. Antibodies raised against this protein detected a truncated form with a molecular mass of 39 kDa. Both forms were predominantly associated with immunoaffinity-isolated apoA-I-containing lipoproteins and detected mainly in the density range 1.123<d<1.21 g/ml. Free apoL was not detected in plasma. ApoL-containing lipoproteins (Lp(L)) showed two major molecular species with apparent diameters of 12.2-17 and 10.4-12.2 nm in the plasma. Moreover, Lp(L) exhibited both pre- and electromobility.

Mainly associated with apoA-I-containing lipoproteins, apo L is a marker of distinct HDL subpopulations. In an effort to gain inference as to its as yet unknown function, Duchateau et al. (2000, J Lipid Res 41:1231-6) studied the biological determinants of apoL levels in human plasma. The distribution of apoL in normal subjects is asymmetric, with marked skewing toward higher values. No difference was found in apoL concentrations between males and females, but they observed an elevation of apoL in primary hypercholesterolemia (10.1 vs. 8.5 microgram/mL in control), in endogenous hypertriglyceridemia (13.8 microgram/mL, P<0.001), combined hyperlipidemia phenotype (18.7 g/mL, P<0.0001), and in patients with type II diabetes (16.2 microgram/mL, P<0.02) who were hyperlipidemic. Significant positive correlations were observed between apoL and the log of plasma triglycerides in normolipidemia (0.446, P<0.0001), endogenous hypertriglyceridemia (0.435, P<0.01), primary hypercholesterolemia (0.66, P<0.02), combined hyperlipidemia (0.396, P<0.04), hypo-alphalipoproteinemia (0.701, P<0.005), and type II diabetes with hyperlipidemia (0.602, P<0.01). Apolipoprotein L levels were also correlated with total cholesterol in normolipidemia (0.257, P<0.004), endogenous hypertriglyceridemia (0.446, P=0.001), and non-insulin-dependent diabetes mellitus (NIDDM) (0.548, P<0.02). No significant correlation was found between apoL and body mass index, age, sex, HDL-cholesterol or fasting glucose and glycohemoglobin levels. ApoL levels in plasma of patients with primary cholesteryl ester transfer protein deficiency significantly increased (7.1+/−0.5 vs. 5.47+/−0.27, P<0.006).

The NOV14 nucleic acids and proteins of the invention have applications in the diagnosis and/or treatment of various diseases and disorders. For example, the compositions of the present invention will have efficacy for the treatment of patients suffering from: premature coronary heart disease, hypercholesterolemia, endogenous hypertriglyceridemia, hyperlipidemia, type II diabetes, Alzheimer's, dysbetalipoproteinemia, hyperlipoproteinemia type III, atherosclerosis, xanthomatosis, premature coronary and/or peripheral vascular disease, hypothyroidism, systemic lupus erythematosus, diabetic acidosis, familial amyloidotic polyneuropathy, Down syndrome as well as other diseases, disorders and conditions.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV14 protein has multiple hydrophilic regions, each of which can be used as an immunogen. In one embodiment, a contemplated NOV14 epitope is from about amino acids 2 to 4. In another embodiment, a contemplated NOV14 epitope is from about amino acids 30 to 40. In other specific embodiments, contemplated NOV14 epitopes are from about amino acids 60 to 80, 105 to 145, 250 to 260, 270 to 290, 305 to 330 and 360 to 380.

NOV15

A disclosed NOV15 (designated CuraGen Acc. No. CG57146-01), which encodes a novel Rh type C Glycoprotein-like protein and includes the ¹³⁵I nucleotide sequence (SEQ ID NO:41) is shown in Table 15A. An open reading frame for the mature protein was identified beginning with an CAG initiation codon at nucleotides 1-3 and ending with a TGG stop codon at nucleotides 1336-1338. Putative untranslated regions are underlined in Table 15A, and the start and stop codons are in bold letters.

TABLE 15A
NOV15 Nucleotide Sequence

(SEQ ID NO:41)

CAGCTGCCCTCCTTCAGGGGGCCAAGTCCCTGGAACTCACCTCCCAGTAGACCGCATCCTCAAAGCAG
TTCTCATCTGAAGGTTGTCCCCAGAATGGTAATCTCAAAATGAGCCCCACAATGATGCCACCCATCAG
GGCCATGGCCAGGGTCACCAAGAGACCATAAATCTGGAACTTTCCCTGTGTTCTTGCGGTCCAGTCCC
CGTTGAAACCTTGAAAGTCAAAGGAATGGACAAGCCCTTCTTTTCCATAGACTTCAAGGCTGGCGGAG
GCCGCTGTCACAGCACCCACGATGCCGCCTATGATGCCAGGAATGCCATGCAGATTGTTAATGCCACA
TGTGTCCTGGATGTGCAGCCGGGACTCCAGGAATGGGGTCAGGTATACAAAACCCAGGGTGGAGATGA
TGCCGCAGACGAAGCCGATGATGAGGGCACCGTAAGGCATGAGCATCATCTCAGCAGCGGTACCCACG
GCCACCCCTCCTGCGAGCGTGGCATTCTGGATGTGCACCATGTCCAGCTTGCCCTTCTTGTGCAGGGC
ACTGGATATTGCCACCGAGGTAAGCACGCAGGCTGCCAAGGAGCAGTAGGTGTTGATGGCGGCTCGGT
GCTGGCTGTCCCCATGGTAGGATATGGCTGAGTTGAAGCTGGGCCAGTACATCCACAGGAAGAGGGTG
CCAATCATGGCAAAGAGGTCCGACTGGTACACAGAATTCTGTCTCTCCTTGCTCTGCTCTAGGTTGCG
TCGGTAGAGGATCCGGGTCACTGTGAGCCCAAAGTAGGCGCCAAATGTGTGGATGGTCATGGAGCCTC
CTGCATCCTTCACCTTTAGCAGGTTAAGGAGAATGAACTCATTCACAGCGAAGAGGGTCACTTGGAAG
AAAGTCATGATGAGCAGCTGAATGGGGCTGACTTTACCCAGAACTGCCCCAAAGGCCACGCAGACAGA
GGCCACGCAGAAGTCAGCGTTGATGAGGTTCTCCACGCCCACGACGATGTAGCGGTCTTGTAAGAAGT
GGAACCAGCCCTGCATGAGCAGCGCCCACTGGATGCCGAAGGCTGCCAACAGGAAGTTGAAGCCCACG
GCGCTGAAGCCGTAGCGCTGCAGGAAAGTCATGAGGAAGCCGAAGCCCACGAAGACCATCACGTGCAC
GTCCTGGAAGCTTGGGTAGCGATAGTAGAATTCGTTCTCCATGTCGCTCAAGTTCTTGTGCGTCCTCT
CTGACCACCAGTGGGCGTCGGCCTCGAAGTCGTAGCGCACGAACACCCCGAAGAGAATCACCATAATC
ACCTGCAGGAGCAGGCAGGTGAGCGGCAGCCGCCAGCGGAGGTTGGTGTTCCAGGCCAT

The disclosed NOV15 nucleic acid sequence maps to chromosome 15q25 and has 1319 of 1325 bases (99%) identical to a gb:GENBANK-ID:AF193809|acc:AF193809.1 mRNA from Homo sapiens (Homo sapiens Rh type C glycoprotein (RHCG) mRNA, complete cds) (E=7.8e⁻²⁹¹).

The disclosed NOV15 polypeptide (SEQ ID NO:42) is 445 amino acid residues in length and is presented using the one-letter amino acid code in Table 51. The SignalP, Psort and/or Hydropathy results predict that NOV15 has a signal peptide and is likely to be localized to the endoplasmic reticulum (membrane) with a certainty of 0.6850. In alternative embodiments, a NOV15 polypeptide is located to the plasma membrane with a certainty of 0.6400, the Golgi body with a certainty of 0.4600, or the endoplasmic reticulum (lumen) with a certainty of 0.1000. The SignalP predicts a likely cleavage site for a NOV15 peptide between amino acid positions 32 and 33, i.e. at the sequence VRY-DF.

TABLE 15B
Encoded NOV15 Protein Sequence

(SEQ ID NO:42)

MAWNTNLRWRLPLTCLLLQVIMVILFGVFVRYDFEADAHWWSERTHKNLSDMENEFYYRYPSFQDVHVMVFV
GFGFLMTFLQRYGFSAVGFNFLLAAFGIQWALLMQGWFHFLQDRYIVVGVENLINADFCVASVCVAFGAVLGK
VSPIQLLIMTFFQVTLFAVNEFILLNLLKVKDAGGSMTIHTFGAYFGLTVTRILYRRNLEQSKERQNSVYQSD
LFAMIGTLFLWMYWPSFNSAISYHGDSQHRAAINTYCSLAACVLTSVAISSALHKKGKLDMVHIQNATLAGGV
AVGTAAEMMLMPYGALIIGFVCGIISTLGFVYLTPFLESRLHIQDTCGINNLHGIPGIIGGIVGAVTAASASL
EVYGKEGLVHSFDFQGFNGDWTARTQGKFQIYGLLVTLAMALMGGIIVGLILRLPFWGQPSDENCFEDAVYWE
VSSRDLAP

The NOV15 amino acid sequence was found to have 437 of 438 amino acid residues (99%) identical to, and 438 of 438 amino acid residues (100%) similar to, the 479 amino acid residue ptnr:SPTREMBL-ACC:Q9UBD6 protein from Homo sapiens (Human) (RH TYPE C GLYCOPROTEIN) (E=8.3e⁻²³⁹).

NOV15 is expressed in at least the following tissues: mammary gland, brain, kidney, testis. Expression information was derived from the tissue sources of the sequences that were included in the derivation of the sequence of NOV15.

Possible small nucleotide polymorphisms (SNPs) found for NOV15 are listed in Table 15C.

TABLE 15C
SNPs

| | Nucleotide | | Amino Acid | |
|---|---|---|---|---|
| Variant | Position | Base Change | Position | Base Change |
| 13377000 | 215 | T > G | 72 | Val > Gly |
| 13377001 | 497 | A > G | 166 | Glu > Gly |
| 13377002 | 1205 | T > C | 402 | Leu > Pro |

NOV15 also has homology to the amino acid sequences shown in the BLASTP data listed in Table 15D.

TABLE 15D
BLAST results for NOV15

| Gene Index/ | | Length | Identity | Positives | |
|---|---|---|---|---|---|
| Identifier | Protein/Organism | (aa) | (%) | (%) | Expect |
| gi\|7706683\|ref\| | Rh type C | 479 | 437/438 | 438/438 | 0.0 |
| NP_057405.1\|(NM_016321) | glycoprotein [Homo | | (99%) | (99%) | |
| | sapiens] | | | | |
| gi\|9790197\|ref\| | Rh type C | 498 | 354/439 | 397/439 | 0.0 |
| NP_062773.1\|(NM_019799) | glycoprotein [Mus | | (80%) | (89%) | |
| | musculus] | | | | |
| gi\|14486157\|gb\| | Rh type C | 459 | 342/439 | 390/439 | 0.0 |
| AAK14650.1\|(AY013260) | glycoprotein [Bos | | (77%) | (87%) | |
| | taurus] | | | | |
| gi\|14486163\|gb\| | Rh type C | 467 | 327/439 | 389/439 | 0.0 |
| AAK14653.1\|(AY013263) | glycoprotein | | (74%) | (88%) | |
| | [Oryctolagus | | | | |
| | cuniculus] | | | | |
| gi\|10039355\|dbj\| | 50 kD glycoprotein | 488 | 272/441 | 349/441 | e−159 |
| BAB13346.1\|(AB036511) | [Oryzias latipes] | | (61%) | (78%) | |

The homology of these sequences is shown graphically in the ClustalW analysis shown in Table 15E

[image]

[image]

Table 15F lists the domain description from DOMAIN analysis results against NOV15. This indicates that the NOV15 sequence has properties similar to those of other proteins known to contain this domain.

TABLE 15F
Domain Analysis of NOV15

gnl|Pfam|pfam00909, Ammonium_transp, Ammonium Transporter Family.
CD-Length = 395 residues, 94.4% aligned
Score = 166 bits (419), Expect = 3e−42

| | | | | |
|---|---|---|---|---|
| NOV15: | 48 | NLSDMENEFYYRYPSFQDVH--VMVFVGFGFLMTFLQRYGFSAVGFNFLLAAFGIQWALL | 105 | (SEQ ID NO:249) |
| | | \| +\| \| +\|\|\|\| \|+ + \|\|\|+ + \| \| \|\| \| \| \| \|\|\|\| \| | | |
| Sbjct: | 23 | GLVRSKNVLNILYKNFQDVAIGVLAYWGFGYSLAFGDSY-FSGFIGNLGLLAAGIQWGTL | 81 | (SEQ ID NO:250) |
| NOV15: | 106 | MQGWFHFLQDRY--IVVGVENLINADFCVASVCVAFGAVLGKVSPIQLLIMTFFQVTLFA | 163 | |
| | | \| \| \| + + + + \|+ \| + \| \|+\|\| + \| + + \| | | |
| Sbjct: | 82 | PDGLFFLFQLMFAATAITIISGAVAERIKFSAYLLFSALLGTLVYPPVAHWVWGEGGWLA | 141 | |
| NOV15: | 164 | VNEFILLNLLKVKDAGGSMTIHTFGAYFGLTVTRILYRRNLEQSKERQNSVYQSDLFAMI | 223 | |
| | | ++ \| \|\| +\| \|\| \| \|\| +\| \| +\| + + \|\|++ | | |
| Sbjct: | 142 | KLGVLV-------DFAGSTVVHIFGGYAGLAAALVLGPRIGRFTKN-EAITPHNLPFAVL | 193 | |
| NOV15: | 224 | GTLFLWMYWPSFNSAISYHGDSQHR-AAINTYCSLAACVLTSVAISSALHKKGKLDMVHI | 282 | |
| | | \|\|\| \|\| \| \|\|+ + \| + \| \|\|+\|\| + \| \|\|++ \|\| \| \|\| +\|+ + | | |
| Sbjct: | 194 | GTLLLWFGWFGFNAGSALTADGRARAAAVNTNLAAAGGALTALLISR--LKTGKPNMLGL | 251 | |
| NOV15: | 283 | QNATLAGGVAVGTAAEMMLMPYGALIIGFVCGIISTLGFVYLTPFLESRLHIQDTCGINN | 342 | |
| | | \| \|\|\| \|\|+ \| ++ \|+\|\|\|\|\|\| + \|++\| \|\|+ \|+ +\| \| \| + | | |
| Sbjct: | 252 | ANGALAGLVAITPAC-GVVSPWGALIIGLIAGVLSVLGY-----KLKEKLGIDDPLDVFP | 305 | |
| NOV15: | 343 | LHGIPGIIGGIVGAVTAASASLEVYGKEGLVHSFDFQGFNGDWTARTQGKFQIYGLLVTL | 402 | |
| | | +\|\|+ \|\| \|\|\| + \|\| \|\|+ + \| \|+ \|+ \| \| | | |
| Sbjct: | 306 | VHGVGGIWGGIAVGIFAALYVNTSGIYGGLL-----------YGNSKQLGVQLIGIAVIL | 354 | |
| NOV15: | 403 | AMALMGGIIVGLI------LRLPFWGQ--PSDENCFEDAVY | 435 | |
| | | \| \| \|+\|\|+ \|\|+ + \| + \| | | |
| Sbjct: | 355 | AYAFGVTFILGLLLGLTLGLRVSEEEEKVGLDLAEHGETAY | 395 | |

A number of evolutionarily-related proteins have been found to be involved in the transport of ammonium ions across membranes. See InterPro IPR001905. Members of this family include Yeast ammonium transporters MEP1, MEP2 and MEP3, Arabidopsis thaliana high affinity ammonium transporter (gene AMTI), Corynebacterium glutamicum ammonium and methylammonium transport system, Escherichia coli putative ammonium transporter amtB. Bacillus subtilis nrgA, Mycobacterium tuberculosis hypothetical protein MtCY338.09c, Synechocystis strain PCC 6803 hypothetical proteins sll0108, sll0537 and sll1017, Methanococcus jannaschii hypothetical proteins MJ0058 and MJ1343, and Caenorhabditis elegans hypothetical proteins C05E11.4, F49E11.3 and M195.3.

As expected by their transport function, these proteins are highly hydrophobic and seem to contain from 10 to 12 transmembrane domains.

The protein similarity information, expression pattern, cellular localization, and map location for the NOV15 protein and nucleic acid disclosed herein suggest that this Rh type C Glycoprotein-like protein may have important structural and/or physiological functions characteristic of the Rh type C Glycoprotein family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The Rh blood group antigens are associated with human erythrocyte membrane proteins of approximately 30 kD, the so-called Rh30 polypeptides. Heterogeneously glycosylated membrane proteins of 50 and 45 kD, the Rh50 glycoproteins, are coprecipitated with the Rh30 polypeptides on immunoprecipitation with anti-Rh-specific mono- and polyclonal antibodies. The Rh antigens appear to exist as a multisubunit complex of CD47, LW, glycophorin B, and play a critical role in the Rh50 glycoprotein.

Ridgwell et al. (1992) isolated cDNA clones representing a member of the Rh50 glycoprotein family, the Rh50A glycoprotein. The cDNA clones containing the full coding sequence of the Rh50A glycoprotein predicted a 409-amino acid N-glycosylated membrane protein with up to 12 transmembrane domains. It showed clear similarity to the Rh30A protein in both amino acid sequence and predicted topology. The findings were considered consistent with the possibility that the Rh30 and Rh50 groups of proteins are different subunits of an oligomeric complex which is likely to have a transport or channel function in the erythrocyte membrane. By analysis of somatic cell hybrids, they mapped the Rh50A gene to 6p21-qter, indicating that genetic differences in the genes for the Rh30 polypeptide, rather than the Rh50 genes, specify the major polymorphic forms of the Rh antigens, because the Rh blood group maps to chromosome 1, not chromosome 6. Cherif-Zahar et al. (1996) carried out 5 regional assignments of the Rh50 gene by isotopic in situ hybridization and concluded that it maps to 6p21.1-p11, probably 6p12.

The Rh(null) types, Rh(null) regulator and Rh(mod) (in which trace amounts of Rh antigens are found), exhibit the same clinical abnormalities associated with chronic hemolytic anemia, stomatocytosis and spherocytosis, reduced osmotic fragility, and increased cation permeability. In addition, Rh(null) membranes characteristically have hyperactive membrane ATPases and reduced red cell cation and water content. Cherif-Zahar et al. (1996) proposed that mutant alleles of Rh50 are suppressors of the RH locus and account for most cases of Rh-deficiency. They analyzed the genes and transcripts encoding Rh, CD47, and Rh50 proteins in 5 unrelated Rh(null) cases and identified 3 types of Rh50 mutations in the transcripts and genomic DNA from them. The first mutation was observed in homozygous state in 2 apparently unrelated individuals originating from South Africa and involved a 2-bp transversion and a 2-bp deletion, introducing a frameshift after the codon for tyrosine-51 (180297.0001). They stated that, since the Rh50 glycoprotein was not detectable by flow cytometry or Western blot analysis on the red cells of these 2 individuals, it is likely that the predicted truncated Rh50 polypeptide (107 residues instead of 409) from these variants was degraded and not inserted into the membrane. The second mutation consisted of a single base deletion at nucleotide 1086, resulting in a frameshift after the codon for alanine-362 (180297.0002). The deduced Rh50 protein was 376 amino acids long (instead of 409) and included 14 novel residues at its C terminus. Surprisingly, this mutation was found in the heterozygous state by RFLP analysis. Attempts to amplify the product of the second Rh50 allele were unsuccessful, strongly suggesting that this transcript was either absent or poorly represented in reticulocytes. Cherif-Zahar et al. (1996) assumed that this allele was transcriptionally silent and that the subjects erythrocytes should carry half the normal dose of a truncated Rh50 protein. Interestingly, flow cytometry and Western blot analysis indicated a complete absence of the protein. They noted that RH and Rh50 proteins interact with each other and suggested that the C terminus of Rh50 may stabilize this interaction or may represent a site of protein-protein interaction critical for cell surface expression.

The third Rh50 mutation identified by Cherif-Zahar et al. (1996) was a missense mutation caused by a G236A transition (180297.0003). Flow cytometry and Western blot analysis indicated that the mutant protein was expressed at the cell surface at only 20% of the wild type level. Cherif-Zahar et al. (1996) provided a diagram of the implication of the 3 mutations in 4 patients with the Rh(null) phenotype of the regulator type. In the fifth subject with Rh(null) phenotype studied by Cherif-Zahar et al. (1996), all attempts to amplify the Rh50 transcript were unsuccessful, although Rh, CD47, and LW sequences were easily amplified and sequenced from reticulocyte RNAs. This suggested that the Rh50 gene was transcriptionally silent in this variant, as had been observed in 1 allele of the subject with the deletion of nucleotide 1086. Findings in these cases indicated to the authors that Rh antigens are significantly expressed only when Rh50 proteins are present. Cherif-Zahar et al. (1996) stated, however, that the converse is not true; a small amount of Rh50 may reach the cell surface in the absence of Rh proteins as indicated by the Rh(null) variant of the silent type. The identification of different Rh50 mutations may account for the well known heterogeneity of Rh(null) individuals classified as regulator and Rh(mod) types.

Huang et al. (1998) described compound heterozygosity for 2 mutations in the Rh50 glycoprotein gene. An 836G-A mutation in exon 6 resulted in a gly279-to-glu substitution, changing a central amino acid of the transmembrane segment 9. While cDNA analysis showed expression of the 836A allele only, genomic studies showed the presence of both 836A and 836G alleles. A detailed analysis of gene organization led to the identification in the 836G allele of a defective donor splice site, caused by a G-to-A mutation in the invariant GT element of the splice donor site of intron 1.

The Rh(mod) syndrome is a rare genetic disorder thought to result from mutations at a ‘modifier’ separate from the suppressor underlying the regulator type of Rh(null) disease, ie., 110 the RHAG gene. Huang et al. (1999) studied this disorder in a Jewish family with a consanguineous background and analyzed RH and RHAG, the 2 loci that control Rh-antigen expression and Rh-complex assembly. Despite the presence of a d (D-negative) haplotype, no other gross alteration was found at the RH locus, and cDNA sequencing showed a normal structure of D, Ce, and ce Rh transcripts in family members. However, analysis of the RHAG transcript identified a single G-to-T transversion in the initiation codon, causing a missense amino acid change: ATG (met) to ATT (ile) (180297.0007).

Huang (1998) determined the intron/exon structure of the Rh50 gene. The structure of the Rh50 gene is nearly identical to that of the Rh30 gene. Of the 10 exons assigned, conservation of size and sequence was confined mainly to the region from exons 2 to 9, suggesting that RH50 and RH30 were formed as 2 separate genetic loci from a common ancestor via a transchromosomal insertion event.

The absence of the RhAG and Rh proteins in Rh(null) individuals leads to morphologic and functional abnormalities of erythrocytes, known as the Rh-deficiency syndrome. The RhAG and Rh polypeptides are erythroid-specific transmembrane proteins belonging to the same family (36% identity). Marini et al. (1997) and Matassi et al. (1998) found significant sequence similarity between the Rh family proteins, especially RhAG, and Mep/Amt ammonium transporters. Marini et al. (2000) showed that RhAG and also RhGK (605381), a human homolog expressed in kidney cells only, function as ammonium transport proteins when expressed in yeast. Both specifically complement the growth defect of a yeast mutant deficient in ammonium uptake. Moreover, ammonium efflux assays and growth tests in the presence of toxic concentrations of the analog methylammonium indicated that RhAG and RhGK also promote ammonium export. The results provided the first experimental evidence for a direct role of RhAG and RhGK in ammonium transport and were of high interest, because no specific ammonium transport system had been previously characterized in human.

Heitman and Agre (2000) diagrammed the phylogenetic tree of multiple sequences from human Rh blood group antigens, human Rh glycoproteins, nonhuman sequences with Rh homology, and ammonium transporters from yeast, bacteria, plants, and worms. In 2 apparently unrelated subjects originating from South Africa and showing the Rh(null) phenotype of the regulator type (268150), Cherif-Zahar et al. (1996) found that nucleotide 154-157 was changed from CCTC to GA (a 2-bp transversion and a 2-bp deletion), introducing a frameshift after the codon for tyrosine-51 and resulting in a premature stop codon at codon 107.

In a subject with Rh(null) of the regulator type (268150), Cherif-Zahar et al. (1996) found heterozygosity for a deletion of adenine-1086 which introduced a frameshift after the codon for alanine-362 and resulted in a premature stop codon at codon 376. In a subject with Rh(null) of the ‘mod’ type (268150), Cherif-Zahar et al. (1996) found a missense mutation, ser79 to asn, caused by a G-to-A transition at nucleotide 236. The other allele was apparently silent.

Hyland et al. (1998) reported molecular findings in the case of an Rh(null) (268150) individual, Y.T., for whom the regulator or amorph type had never been formally documented, although the donor's cells were used in several biochemical studies. Preliminary family studies showed that functional D and C antigens were transmitted from Y.T. to 3 children, suggesting that Y.T. belonged to the regulator type. Molecular studies showed that Y.T. inherited the mutation from her mother and was a compound heterozygote (composite heterozygote in the terminology of Hyland et al., 1998), carrying 1 mutant Rh50 allele and 1 transcriptionally silent Rh50 allele. The Rh50 mRNA was found to contain an 836G-A transition yielding a missense and nonconservative gly279-to-glu (G279E) amino acid substitution within a predicted hydrophobic domain of the membrane protein. Y.T. was found by study of genomic DNA to be carrying both an 836A allele and an 836G allele but only the 836A sequence was represented in cDNA, indicating that the 836G allele was silent.

Huang et al. (1998) demonstrated compound heterozygosity of the Rh50 gene as the basis of the Rh(null) phenotype. One mutation was an 836G-A mutation resulting in a missense change, gly279 to glu, in exon 6. The other mutation was a change of the invariant GT element of the splice donor site of intron 1 to AT. The blood sample in this case was from a female proband (Y.T.) of Australian origin. Serologic tests confirmed the null status of Rh antigens (D-C-E-c-e- and Rh17-). See 180297.0004 and Huang et al. (1998). The same mutation was found by Cherif-Zahar et al. (1998) in homozygous state in a patient in California with Rh(null) of the regulator type (268150). Cherif-Zahar et al. (1998) described splicing mutations in the Rh50 gene in 2 unrelated patients with the ‘typical Rh(null) syndrome’ (268150). The first mutation affected the invariant G residue of the 3-prime acceptor splice site of intron 6, causing the skipping of the downstream exon and the premature termination of translation. The second mutation occurred at the first base of the 5-prime donor splice site of intron 1 (180297.0005). Both of these mutations were found in homozygous state.

In a Jewish family of Russian origin with a consanguineous background, Huang et al. (1999) found that the basis of the Rh(mod) syndrome was a met-to-ile mutation in the initiation codon of the RHAG transcript. This point mutation occurred in the genomic region spanning exon 1 of RHAG. The presence of the mutation in the mother and 2 children was confirmed by SSCP analysis. Although blood typing showed a very weak expression of Rh antigens, immunoblotting barely detected the Rh proteins in Rh(mod) membrane. In vitro transcription-coupled translation assays showed that the initiator mutants of Rh(mod), but not those of the wild type, could be translated from ATG codons downstream. The findings pointed to incomplete penentrance of the Rh(mod) mutation, in the form of ‘leaky’ translation, leading to some posttranslational defects affecting the structure, interaction, and processing of Rh50 glycoprotein. The mother in this pedigree (S.M.) and her brother (S.S.) were first described as cases of Rh(null). S.M. had a well-compensated hemolytic anemia, whereas S.S. had a normal hematologic count with numerous spherocytes and stomatocytes after splenectomy. S.M. was found to be homozygous for the mutation; SS was deceased at the time of study. The 2 children of S.M. were heterozygotes.

In 1 patient with Rh-null disease of the regulator type (268150), Huang (1998) detected a shortened Rh50 transcript lacking the sequence of exon 7. They identified a G-to-A transition at the +1 site of IVS7 in homozygosity in this patient. This splicing mutation caused not only a total skipping of exon 7 but also a frameshift and premature chain termination. Thus, the deduced translation product contained 351 instead of 409 amino acids, with an entirely different C-terminal sequence following thr315. Huang et al. (1999) demonstrated that a Japanese patient with Rh-null hemolytic anemia of the regulator type (268150) was homozygous for 2 cis mutations in the RHAG gene: in exon 6, G-to-A transitions, GTT to ATT and GGA to AGA, which caused val270-to-ile and gly280-to-arg substitutions, respectively. In a Japanese patient with Rh-null hemolytic anemia of the regulator type (268150), Huang et al. (1999) identified a G-to-T transversion in exon 9 of the RHAG gene, converting GGT (gly) to GTT (val) at codon 380 in the transmembrane-12 segment. The transversion, which was located at the +1 position of exon 9, had also affected pre-mRNA splicing and caused partial exon skipping. Despite a structurally normal Rh antigen locus, hemagglutination and immunoblotting showed no expression of Rh antigens or proteins.

See: Cherif-Zahar, et al., Blood 92: 2535-2540, 1998. PubMed ID: 9746795; Cherif-Zahar, et al., Nature Genet. 12: 168-173, 1996. PubMed ID: 8563755; Heitman and Agre, Nature Genet. 26: 258-259, 2000. PubMed ID: 11062455; Huang, C.-H., J. Biol. Chem. 273: 2207-2213, 1998. PubMed ID: 9442063.1; Huang, et al., Am. J. Hemat. 62: 25-32, 1999. PubMed ID: 10467273; Huang, et al., Am. J. Hum. Genet. 64: 108-117, 1999. PubMed ID: 9915949; Huang, et al., Blood 92: 1776-1784, 1998. PubMed ID: 9716608; Hyland, et al., Blood 91: 1458-1463, 1998. PubMed ID: 9454778; Marini, et al., Nature Genet. 26: 341-344, 2000. PubMed ID: 11062476; Marini, et al., Trends Biochem. Sci. 22: 460461, 1997. PubMed ID: 9433124; Matassi, et al., Genomics 47: 286-293, 1998. PubMed ID: 9479501; and Ridgwell, et al., Biochem. J. 287: 223-228, 1992. PubMed ID: 1417776.

The NOV15 nucleic acids and proteins of the invention have applications in the diagnosis and/or treatment of various diseases and disorders. For example, the compositions of the present invention will have efficacy for the treatment of patients suffering from: hemolytic anemia, stomatocytosis and spherocytosis, reduced osmotic fragility, and increased cation permeability; Rh(mod) syndrome, Rh(null)disease; Rh deficiency syndrome; ammonium transport; Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration; fertility, hypogonadism; diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome; Glutaricaciduria, type IIA; Hypercholesterolemia, familial, autosomal recessive; Tyrosinemia, type I as well as other diseases, disorders and conditions.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV15 protein has multiple hydrophilic regions, each of which can be used as an immunogen. In one embodiment, a contemplated NOV15 epitope is from about amino acids 40 to 55. In another embodiment, a contemplated NOV15 epitope is from about amino acids 195 to 215. In other specific embodiments, contemplated NOV15 epitopes are from about amino acids 240 to 255, 290 to 295, 340 to 345 and 360 to 365.

NOV16

A disclosed NOV16 (designated CuraGen Acc. No. CG57169-01), which encodes a novel Copine III-like protein and includes the 1763 nucleotide sequence (SEQ ID NO:43) is shown in Table 16A. An open reading frame for the mature protein was identified beginning with an CTG initiation codon at nucleotides 111-113 and ending with a TAT stop codon at nucleotides 1758-1760. Putative untranslated regions are underlined in Table 16A, and the start and stop codons are in bold letters.

TABLE 16A
NOV16 Nucleotide Sequence

(SEQ ID NO:43)

AGCTCAGGTCGGGTTCTCGTAGCTGGTGGGGGGCAGGTTTTTATGCTTGAAATACTGCACAACTTGTTGGGGCAGCTC
CGCCAGCACAGCTTTGGCCAAGGTCTCTTTTGCTGCGTTGCGGAACTCTCGAAAGGGAACGAACTGCACAATATCGCG
GGCTGCCTCCTCCCCCGTGTGGGAGCGCAGCATGCGGCTGTCCCCATCCAGGAACTCCATGGCAGCGAAGTCCGCATT
GCCCACGCCCACGATGATGATGGACATGGGCAGCTTGGAAGCCTGCACCACGGCATGCCGTGTCTCCTCCATGTCACT
GATGACCCCGTCCGTGATGATGAGGAGGATGAAGTACTGCGTGGCCGTCCGCTGTTGTGTGGCCTGGGCCGCAAACCG
GGCCACGTGGTTGACGATGGGGGAGAAATTGGTAGGACCGTAGAAGCGGATGTGGGGCAGGCAAGCTGAGTACGCCTG
GGCAATACCATCCACACCTGAGCAGAAGGGGTTGGTGGGGTTGAAGTTGATGGCAAACTCATGGGAGACCTTCCAGTC
TGGGGGTAACTGGGCCCCGAATCCCAGAGCTGGAAACATCTTATCACTGTCGTAGTCCTGAATGATCTGCCCAACAGC
CCAGATGGCCGACAGATATTCGTTGGTGCCCATAGGGTTGATATAGTGCAAAGAGGAAGGGTCGAGGGGATTCCCGTT
GGAGGCTGTAAAGTCTATTCCAACGGTGAACATGAGCTGGCAGCCTCCCAGGATGTAGTCAAGGAAGGAGTAGTCTCG
GTTTATCTTGCAGGATCGCAGGATGATGATGCCCGAGTTTTTATAGTTCTTCTTCTTCCTCTGCTTCTTGGGGTTGAT
GCACTCGAACTCCAGCGGGACGCTGTCTCGAGCCTCACACATCTGTGACACTGAGGTCTGGAACTCGCCGATGAAGTC
ATGGCCCCCGTCATTGTCATAGTCGTAGCACATGACCTGGATGGGCTTCTCCATGTCCCCATCACACAGGGACACCAA
GGGCACTGTGAATGGCTTCCACACAGGGTCCAGTGTGTACTTGATCACCTCAGTCCTGTGGACCAGCATCCACTTGCC
ATCGTCTCCTGGCTTATAAAACTCCAGAAAGGGGTCTGACTTCCCAAAGAGGTCCTTCTTGTCCAGCCTCCTGCCCGC
CAGGCTTAGTGTGATGACGCGGTTGTCGGACAGCTCCTGGGCAGCGATCGTAATCAAGCCCTTCCCCGCAGGCTTGTC
ATTCAGCAGCAGCAGAGGCCTAGTGATCTTCTTGCTGGAGACGATCGTGCCCAGGCTGCAGGAGAACTGGCCCAGGAA
GTCATGCTCGTCCAGCCGCATACTGGACTTGTCCTGGTCAAAGAGCGCGAACTTGAGCTTCTGTACCTCCTCGAAGTG
GTAGTCAAGCACGAACTTCTTGGAGAAGGCGGGGTTGAGGTTGTTGATCGCGGTTTCTGTCCTGTCCTACTCGATCCA
TCTGCCATTGTTCTCTGTAAAGAGGACACAGAAGGGGTCGGACTTGGAGGTAACATCCCGGTCCAGTAGGTTCTGGCC
ACTCACTGACAGCTCCACCTTGCACACGCAATACTGGGGGCCCATGGGGGCTGCCCCCGCTGCTGGGGCACCCCCACT
GGGTATGTGGGCCATGGGAGCCGGTGGCGGTGGCAGGAGTTCCTGGCAGTCGCAGGTCCCGCGGGCGCCACCGCCCTC
ACCGCACGGCTGCCGCTGCCCGCGCTCCGAGCCACCCGGGGTATCCT

The disclosed NOV16 nucleic acid sequence maps to chromosome 16 and has 924 of 1344 bases (68%) identical to a gb:GENBANK-ID:HSA133798|acc:AJ133798.1 mRNA from Homo sapiens (Homo sapiens mRNA for copine VI protein) (E=1.5e⁻¹²⁴).

A disclosed NOV16 polypeptide (SEQ ID NO:44) is 549 amino acid residues in length and is presented using the one-letter amino acid code in Table 16E. The SignalP, Psort and/or Hydropathy results predict that NOV16 does not have a signal peptide and is likely to be localized to the endoplasmic reticulum (membrane) with a certainty of 0.6850. In alternative embodiments, a NOV16 polypeptide is located to the plasma membrane with a certainty of 0.6400, the Golgi body with a certainty of 0.4600, or the endoplasmic reticulum (lumen) with a certainty of 0.1000.

TABLE 16B
Encoded NOV16 Protein Sequence

(SEQ ID NO:44)

MAHIPSGGAPAAGAAPMGPQYCVCKVELSVSGQNLLDRDVTSKSDPFCVLFTENNGRWIEYDRTETAINNLNPAFSKK
FVLDYHFEEVQKLKFALFDQDKSSMRLDEHDFLGQFSCSLGTIVSSKKITRPLLLLNDKPAGKGLITIAAQELSDNRV
ITLSLAGRRLDKKDLFGKSDPFLEFYKPGDDGKWMLVHRTEVIKYTLDPVWKPFTVPLVSLCDGDMEKPIQVMCYDYD
NDGGHDFIGEFQTSVSQMCEARDSVPLEFECINPKKQRKKKNYKNSGIIILRSCKINRDYSFLDYILGGCQLMFTVGI
DFTASNGNPLDPSSLHYINPMGTNEYLSAIWAVGQIIQDYDSDKMFPALGFGAQLPPDWKVSHEFAINFNPTNPFCSG
VDGIAQAYSACLPHIRFYGPTNFSPIVNHVARFAAQATQQRTATQYFILLIITDGVISDMEETRHAVVQASKLPMSII
IVGVGNADFAAMEFLDGDSRMLRSHTGEEAARDIVQFVPFREFRNAAKETLAKAVLAELPQQVVQYFKHKNLPPTSYE
NPT

The NOV16 amino acid sequence was found to have 341 of 527 amino acid residues (64%) identical to, and 427 of 527 amino acid residues (81%) similar to, the 537 amino acid residue ptnr:SWISSNEW-ACC:075131 protein from Homo sapiens (Human) (COPINE III) (E=5.1e⁻¹⁹³).

NOV16 is expressed in at least the following tissues: Bone, Brain, Ovary, Spinal Chord, and Uterus. Expression information was derived from the tissue sources of the sequences that were included in the derivation of the sequence of NOV16.

NOV16 also has homology to the amino acid sequences shown in the BLASTP data listed in Table 16C.

TABLE 16C
BLAST results for NOV16

| Gene Index/ | | Length | Identity | Positives | |
|---|---|---|---|---|---|
| Identifier | Protein/Organism | (aa) | (%) | (%) | Expect |
| gi\|14714939\|gb\| | Unknown (protein | 446 | 442/444 | 443/444 | 0.0 |
| AAH10627.1\|AAH10627 (BC010627) | for MGC: 16924) | | (99%) | (99%) | |
| | [Homo sapiens] | | | | |
| gi\|15318878\|ref\| | hypothetical | 358 | 354/356 | 355/356 | 0.0 |
| XP_053605.1\|(XM_053605) | protein XP_053605 | | (99%) | (99%) | |
| | [Homo sapiens] | | | | |
| gi\|4503015\|ref\| | copine III [Homo | 537 | 339/523 | 424/523 | 0.0 |
| NP_003900.1\|(NM_003909) | sapiens] | | (64%) | (80%) | |
| gi\|4503013\|ref\| | copine I [Homo | 537 | 311/531 | 400/531 | 0.0 |
| NP_003906.1\|(NM_003915) | sapiens] | | (58%) | (74%) | |
| gi\|14193684\|gb\| | copine 1 protein | 454 | 267/453 | 351/453 | e−162 |
| AAK56087.1\|AF332058_1 | [Mus musculus] | | (58%) | (76%) | |
| (AF332058) | | | | | |

The homology of these sequences is shown graphically in the ClustalW analysis shown in Table 16D. [image] [image] Table 16E lists the domain description from DOMAIN analysis results against NOV16. This indicates that the NOV16 sequence has properties similar to those of other proteins known to contain these domains.

TABLE 16E
Domain Analysis of NOV16

gnl|Smart|smart00239, C2, Protein kinase C conserved region 2 (CalB); Ca2+-
binding motif present in phospholipases, protein kinases C, and
synaptotamins (among others). Some do not appear to contain Ca2+-binding
sites. Particular C2s appear to bind phospholipids, inositol polyphosphates,
and intracellular proteins. Unusual occurrence in perform. Synaptotagmin
and PLC C2s are permuted in sequence with respect to N- and C-terminal beta
strands. SMART detects C2 domains using one or both of two profiles.
CD-Length = 101 residues, 87.1% aligned
Score = 64.7 bits (156), Expect = 1e−11

| | | | | |
|---|---|---|---|---|
| NOV16: | 161 | LAGRRLDKKDLFGKSDPFLEFYKPGDDGKWMLVHRTEVIKYTLDPVW-KPFTVPLVSLCD | 219 | (SEQ ID NO:256) |
| | | ++ \| \| \|\| \|\|\|\|\|+++ \|\| + +\|+ +\| \|\|+\|\|\| + \| + | | |
| Sbjct: | 7 | ISARNLPPKDRGGKSDPYVKVSLDGDPRE---KKKTKVVKNTLNPVWNETFEFEVPPPEL | 63 | (SEQ ID NO:257) |
| NOV16: | 220 | GDMEKPIQVMCYDYDNDGGHDFIGEFQTSVSQMCE | 254 | |
| | | +++ \|\| \| \|\|\|\| +\| + | | |
| Sbjct: | 64 | ----SELEIEVYDKDRFSRDDFIGRVTIPLSDLLL | 94 | |

CD-Length = 101 residues, 93.1% aligned
Score = 62.4 bits (150), Expect = 7e-11

| | | | | |
|---|---|---|---|---|
| NOV16: | 30 | VSGQNLLDRDVTSKSDPFCVLFTENNGRWIEYDRTETAINNLNPAFSKKFVLDYHFEEVQ | 89 | (SEQ ID NO:258) |
| | | +\| +\|\| +\| \|\|\|\|+ + + + \| \| +\|+ \| \|\|\| +++ \| + \|+ | | |
| Sbjct: | 7 | ISARNLPPKDKGGKSDPYVKVSLDGDPR--EKKKTKVVKNTLNPVWNETFEFEVPPPELS | 64 | (SEQ ID NO:259) |
| NOV16: | 90 | KLKFALFDQDKSSMRLDEHDFLGQFSCSLGTIVSSKKITR | 129 | |
| | | +\|+ ++\|+\|+ \| \|\|+\|+ + \| ++ + + | | |
| Sbjct: | 65 | ELEIEVYDKDRFS----RDDFIGRVTIPLSDLLLGGRHEK | 100 | |

gnl|Pfam|pfam00168, C2, C2 domain.
CD-Length = 88 residues, 93.2% aligned
Score = 56.6 bits (135), Expect = 4e−09

| | | | | |
|---|---|---|---|---|
| NOV16: | 30 | VSGQNLLDRDVTSKSDPFCVLFTENNGRWIEYDRTETAINNLNPAFSKKFVLD-YHFEEV | 88 | (SEQ ID NO:260) |
| | | +\| +\|\| \|+ \|\|\|+ + + + + + +\|+\| \|\|\| +++ \|\| + ++ | | |
| Sbjct: | 6 | ISARNLPKMDMNGLSDPYVKVDLDGDPKDTKKFKTKTVKKTLNPVWNETFVFEKVPLPDL | 65 | (SEQ ID NO:261) |
| NOV16: | 89 | QKLKFALFDQDKSSMRLDEHDFLGQF | 114 | |
| | | \|+\|\|++\|+\|+ \| \|\|+\|\| | | |
| Sbjct: | 66 | ASLRFAVYDEDRFS----RDDFIGQV | 87 | |

CD-Length = 88 residues, 93.2% aligned
Score = 56.6 bits (135), Expect = 4e−09

| | | | | |
|---|---|---|---|---|
| NOV16: | 161 | LAGRRLDKKDLFGKSDPFLEFYKPGDDGKWMLVHRTEVIKYTLDPVW-KPFTVPLVSLCD | 219 | (SEQ ID NO:262) |
| | | ++ \| \| \| \|+ \| \|\|\|+++ \| \| +\|+ +\| \|\|+\|\|\| + \| \| \| \| | | |
| Sbjct: | 6 | ISARNLPKMDMNGLSDPYVKV-DLDGDPKDTKKFKTKTVKKTLNPVWNETFVFEKVPLPD | 64 | (SEQ ID NO:263) |
| NOV16: | 220 | GDMEKPIQVMCYDYDNDGGHDFIGEF | 245 | |
| | | ++ \|\| \| \|\|\|\|+ | | |
| Sbjct: | 65 | L---ASLRFAVYDEDRFSRDDFIGQV | 87 | |

gnl|Smart|smart00327, VWA, von Willebrand factor (vWF) type A domain; VWA
domains in extracellular eukaryotic proteins mediate adhesion via metal ion-
dependent adhesion sites (MIDAS). Intracellular VWA domains and homologues
in prokaryotes have recently been identified. The proposed VWA domains in
integrin beta subunits have recently been substantiated using sequence-based
methods (Pointing et al. Adv Prot Chem (2000) in press).
CD-Length = 180 residues, 92.2% aligned
Score = 40.8 bits (94), Expect = 2e−04

| | | | | |
|---|---|---|---|---|
| NOV16: | 333 | MGTNEYLSAIWAVGQIIQDYDSDKMFPALGFGAQLPPDWKVSHEFAINFNPTNPFCSGVD | 392 | (SEQ ID NO:264) |
| | | \|\| \| + \| \| ++++ \| +\| \| + + \| + + | | |
| Sbjct: | 14 | MGGNRFELAKEFVLKLVEQLDIGPDGDRVGL-------VTFSSDARVLFPLND--SQSKD | 64 | (SEQ ID NO:265) |
| NOV16: | 393 | GIAQAYSACLPHIRFYGPTNFSPIVNHVARFAAQATQQRTATQYFILLIITDGVISD-ME | 451 | |
| | | + +\| ++ \| \|\|\| + + + +\|++\|\|\|\| +\| \| | | |
| Sbjct: | 65 | ALLEALASLSYS--LGGGTNLGAALEYALENLFSESAGSRRGAPKVLILITDGESNDGGE | 122 | |
| NOV16: | 452 | ETRHAVVQASKLPMSIIIVGVGNA-DFAAMEFLDGDSRMLRS-HTGEEAARDIVQFV | 506 | |
| | | + \| + + + + +\|\|\|\|\| \| ++ \| + ++ + | | |
| Sbjct: | 123 | DILKAAKELKRSGVKVFVVGVGNDVDEEELKKLASAPGGVFVVEDLPSLLDLLIDLL | 179 | |

Some isozymes of protein kinase C (PKC) contain a domain, known as C2, of about 116 amino-acid residues which is located between the two copies of the C1 domain (that bind phorbol esters and diacylglycerol) (see PROSITEDOC PDOC00379) and the protein kinase catalytic domain (see PROSITEDOC PDOC00100). Regions with significant homology to the C2-domain have been found in many proteins. The C2 domain is thought to be involved in calcium-dependent phospholipid binding. Since domains related to the C2 domain are also found in proteins that do not bind calcium, other putative functions for the C2 domain like e.g. binding to inositol-1,3,4,5-tetraphosphate have been suggested. The 3D structure of the C2 domain of synaptotagmin has been reported, the domain forms an eight-stranded beta sandwich constructed around a conserved 4-stranded motif, designated a C2 key. Calcium binds in a cup-shaped depression formed by the N- and C-terminal loops of the C2-key motif. The domain information provided in Table 16E indicates that the sequence of the invention has properties similar to those of other proteins known to contain this/these domain(s) and similar to the properties of these domains.

Molecular events at the interface of the cell membrane and cytoplasm may be regulated by proteins that attach to and detach from the membrane surface in response to signals. Calcium-dependent membrane-binding proteins may play such a role. To identify proteins that may underlie membrane trafficking processes in ciliates, Creutz et al. (1998) isolated calcium-dependent phospholipid-binding proteins from Paramecium. They named the major protein that they obtained ‘copine’ (pronounced ‘ko-peen’), the French feminine noun meaning ‘fiend,’ because it associates like a ‘companion’ with lipid membranes. The 55-kD copine protein bound phosphatidylserine in a calcium—but not magnesium-dependent manner, but it did not bind phosphatidylcholine. Copine promoted calcium-dependent aggregation of lipid vesicles. The authors cloned partial cDNAs representing 2 distinct Paramecium copine genes. By searching sequence databases for genes with sequence similarity to the Paramecium copine genes, Creutz et al. (1998) identified human ESTs corresponding to 5 copine genes, named copine I to V. Two overlapping ESTs contained the complete copine I (CPNE1) coding sequence. The deduced 537-amino acid CPNE1 protein contains 2 type II C2 domains in its N-terminal half and a domain similar to the A domain, which is present in a number of extracellular proteins or the extracellular portions of membrane proteins, in its C-terminal half; it does not have a predicted signal sequence or transmembrane domains. C2 domains mediate calcium-dependent interactions with phospholipids, and the A domain of integrins appears to mediate the binding of the integrin to extracellular ligands. CPNE1 has a broad tissue distribution. Recombinant CPNE1 expressed in bacteria exhibited calcium-dependent binding to phosphatidylserine vesicles. Antibody against CPNE1 reacted with bovine chromobindin-17, which is a 55-kD calcium-dependent chromaffin vesicle-binding protein, and the authors concluded that chromobindin-17 is a copine. They suggested that copines function in membrane trafficking. See Creutz, et al., J. Biol. Chem. 273:1393-1402, 1998. PubMed ID: 9430674.2. Ishikawa, et al., DNA Res. 5: 169-176, 1998. PubMed ID: 9734811.

The protein similarity information, expression pattern, cellular localization, and map location for the NOV16 protein and nucleic acid disclosed herein suggest that this Copine III-like protein may have important structural and/or physiological functions characteristic of the Copine III family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV16 nucleic acids and proteins of the invention have applications in the diagnosis and/or treatment of various diseases and disorders. For example, the compositions of the present invention will have efficacy for the treatment of patients suffering from: Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, cancer, trauma, tissue regeneration (in vitro and in vivo), viral/bacterial/parasitic infections, immunological disease, respiratory disease, gastro-intestinal diseases, reproductive health, neurological and neurodegenerative diseases, bone marrow transplantation, metabolic and endocrine diseases, allergy and inflammation, nephrological disorders, cardiovascular diseases, muscle, bone, joint and skeletal disorders, hematopoietic disorders, urinary system disorders, systemic lupus erythematosus, autoimmune disease, asthma, emphysema, scleroderma, allergy, ARDS, fertility, as well as other diseases, disorders and conditions.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV16 protein has multiple hydrophilic regions, each of which can be used as an immunogen. In one embodiment, a contemplated NOV16 epitope is from about amino acids 30 to 90. In another embodiment, a contemplated NOV16 epitope is from about amino acids 95 to 98. In other specific embodiments, contemplated NOV16 epitopes are from about amino acids 99 to 105, 120 to 122, 130 to 132, 140 to 190, 210 to 220, 260 to 290, 320 to 330, 340 to 375, 400 to 410, 420 to 440 and 490 to 550.

NOV17

A disclosed NOV17 (designated CuraGen Acc. No. CG57177-01), which encodes a novel Carboxypeptidase B, Pancreatic-like protein and includes the 1070 nucleotide sequence (SEQ ID NO:45) is shown in Table 17A. An open reading frame for the mature protein was identified beginning with an ATG initiation codon at nucleotides 1-3 and ending with a TAG stop codon at nucleotides 1048-1050. Putative untranslated regions are underlined in Table 17A, and the start and stop codons are in bold letters.

TABLE 17A
NOV17 Nucleotide Sequence

(SEQ ID NO:45)

ATGTTGGCACTCTTGGTTCTGGTGACTGTGGCCCTGGCATCTGCTCATCATGGTGGTGAGCACTTTGAA
GGGGAGAAGGTGTTCCGTGTTAACGTTGAAGATGAAAATCACATTAACATAATCCGCGAGTTGGCCACC
TTTATTCAGATTGACTTCTGGAAGCCAGATTCTGTCACACAAATCAAACCTCACAGTACAGTTGACTTC
CGTGTTAAAGCAGAAGATACTGTCACTGTGGAGAATGTTCTAAAGCAGAATGAACTACAATACAAGGTA
CTGATAAGCAACCTGAGAAATGTGGTGGAGGCTCAGTTTGATAGCCGGGTTCGTGCAACAGGACACAGT
TATGAGAAGTACAACAAGTGGGAAACGATAGAGGCTTGGACTCAACAAGTCGCCACTGAGAATCCAGCC
CTCATCTCTCGCAGTGTTATCGGAACCACATTTGAGGGACGCGCTATTTACCTCCTGAAGGTTGGCAAA
GCTGGACAAAATAAGCCTGCCATTTTCATGGAATGTGGTTTCCATGCCAGAGAGTGGATTTCTCCTGCA
TTCTGCCAGTGGTTTGTAAGAGAGGCTGTTCGTACCTATGGACGTGAGATCCAAGTGACAGAGCTTCTC
GACAAGTTAGACTTTTATGTCCTGCCTGTGCTCAATATTGATGGCTACATCTACACCTGGACCAAGAGC
CGATTTTGGAGAAAGACTTCGCTCCACCCATACTGGATCTACCCTTACTCATATGCTTACAAACTCGGT
GAGAACAATGCTGAGTTGAATGCCCTGGCTAAAGCTACTGTGAAAGAACTTGCCTCACTGCACGGCACC
AAGTACACATATGGCCCGGGAGCTACAACAATCTATCCTGCTGCTGGGGGCTCTGACGACTGGGCTTAT
GACCAAGGAATCAGATATTCCTTCACCTTTGAACTTCGAGATACAGGCAGATATGGCTTTCTCCTTCCA
GAATCCCAGATCCGGGCTACCTGCGAGGAGACCTTCCTGGCAATCAAGTATGTTGCCAGCTACGTCCTG
GAACACCTGTACTAGTTGAGAAAGCTGATGGCCTT

The disclosed NOV17 nucleic acid sequence maps to chromosome 3 and has 626 of 729 bases (85%) identical to a gb:GENBANK-ID:DOGZAP47|acc:D78348.1 mRNA from Canis familiaris (Dog mRNA for zymogen granule membrane associated protein (ZAP47), complete cds) (E=4.0e⁻¹⁷¹).

A disclosed NOV17 polypeptide (SEQ ID NO:46) is 349 amino acid residues in length and is presented using the one-letter amino acid code in Table 17B. The SignalP, Psort and/or Hydropathy results predict that NOV17 does not have a signal peptide and is likely to be localized to the outside of the cell with a certainty of 0.5422. In alternative embodiments, a NOV17 polypeptide is located to the microbody (peroxisome) with a certainty of 0.2456, the endoplasmic reticulum (membrane) with a certainty of 0.1000, or the endoplasmic reticulum (lumen) with a certainty of 0.1000.

TABLE 17B
Encoded NOV17 Protein Sequence

(SEQ ID NO:46)

MLALLVLVTVALASAHHGGEHFEGEKVFRVNVEDENHINIIRELATFIQIDFWKPDSVTQIKPHSTVDFRVKAEDTV
TVENVLKQNELQYKVLISNLRNVVEAQFDSRVRATGHSYEKYNKWETIEAWTQQVATENPALISRSVIGTTFEGRAIY
LLKVGKAGQNKPAIFMECGFHAREWISPAFCQWFVREAVRTYGREIQVTELLDKLDFYVLPVLNIDGYIYTWTKSRFW
RKTSLHPYWIYPYSYAYKLGENNAELNALAKATVKELASLHGTKYTYGPGATTIYPAAGGSDDWAYDQGIRYSFTFEL
RDTGRYGFLLPESQIRATCEETFLAIKYVASYVLEHLY

The NOV17 amino acid sequence was found to have 234 of 240 amino acid residues (97%) identical to, and 236 of 240 amino acid residues (98%) similar to, the 416 amino acid residue ptnr:pir-id:A42332 protein from human (carboxypeptidase B (EC 3.4.17.2) precursor, pancreatic) (E=5.4e⁻⁸²).

NOV17 is expressed in at least the following tissues: pancreas, blood, stomach. Expression information was derived from the tissue sources of the sequences that were included in the derivation of the sequence of NOV17.

Possible small nucleotide polymorphisms (SNPs) found for NOV17 are listed in Table 17C.

TABLE 17C
SNPs

| | Nucleotide | | Amino Acid | |
|---|---|---|---|---|
| Variant | Position | Base Change | Position | Base Change |
| 13374719 | 516 | A > C | 172 | Glu > Asp |

Other NOV17 variants include the nucleic acids depicted in Table 17D and the proteins depicted in Table 17E.

[image]

[image]

[image]

[image]

[image]

[image]

[image]

[image]

[image]

[image]

NOV17 also has homology to the amino acid sequences shown in the BLASTP data listed in Table 17F.

TABLE 17F
BLAST results for NOV17

| Gene Index/ | | Length | Identity | Positives | |
|---|---|---|---|---|---|
| Identifier | Protein/Organism | (aa) | (%) | (%) | Expect |
| gi\|4503003\|ref\| | pancreatic | 416 | 292/416 | 303/416 | e−150 |
| NP_001862.1\|(NM_001871) | carboxypeptidase B1 | | (70%) | (72%) | |
| | precursor; | | | | |
| | pancreas-specific | | | | |
| | protein [Homo | | | | |
| | sapiens] | | | | |
| gi\|15929839\|gb\| | Unknown (protein | 417 | 291/417 | 303/417 | e−150 |
| AAH15338.1\|AAH15338 (BC015338) | for MGC: 21282) | | (69%) | (71%) | |
| | [Homo sapiens] | | | | |
| gi\|3915628\|sp\| | HUMAN | 417 | 290/417 | 303/417 | e−150 |
| P15086\|CBPB_{—} | CARBOXYPEPTIDASE B | | (69%) | (72%) | |
| | PRECURSOR | | | | |
| | (PANCREAS-SPECIFIC | | | | |
| | PROTEIN) (PASP) | | | | |
| gi\|5457422\|emb\| | procarboxypeptidase | 416 | 239/416 | 272/416 | e−122 |
| CAB46991.1\|(AJ133775) | B [Sus scrofa] | | (57%) | (64%) | |
| gi\|1705666\|sp\| | Carboxypeptidase B | 416 | 237/416 | 272/416 | e−122 |
| P55261\|CBPB_CANFA | precursor (47 kDa | | (56%) | (64%) | |
| | zymogen granule | | | | |
| | membrane associated | | | | |
| | protein) (ZAP47) | | | | |

The homology of these sequences is shown graphically in the ClustalW analysis shown in Table 17G.

[image]

[image]

Table 17H lists the domain description from DOMAIN analysis results against NOV17. This indicates that the NOV17 sequence has properties similar to those of other proteins known to contain these domains.

TABLE 17H
Domain Analysis of NOV17

HMM file: pfamHMMs
Scores for sequence family classification (score includes all domains):

| Model | Description | Score | E-value | N |
|---|---|---|---|---|
| Zn_carbOpept | (Interpro) Zinc carboxypeptidase | 357.0 | 2e−103 | 2 |
| Propep_M14 | (InterPro) Carboxypeptid activation pept | 138.1 | 1.6e−37 | 1 |

Parsed for domains:

| | | seq | seq | hmm | hmm | | |
|---|---|---|---|---|---|---|---|
| Model | Domain | from | to | from | to | score | E-value |
| Propep_M14 | 1/1 | 26 | 105 . . . | 1 | 82 [] | 138.1 | 1.6e−37 |
| Zn_carbOpept | 1/2 | 119 | 236 . . . | 1 | 125 [. | 206.6 | 3.8e−58 |
| Zn_carbOpept | 2/2 | 242 | 332 . . . | 204 | 304 .] | 149.5 | 6e−41 |

Alignments of top-scoring domains:
Propep_M14: domain 1 of 1, from 26 to 105: score 138.1, E=1.6e=37

| | | | |
|---|---|---|---|
| | *->qVlrvkvadedQvkllkdLentehleLDFWkpdsatpikpgstvDfr | | (SEQ ID NO: 271) |
| | +\|+\|\|+\|+\|\|++++++++\|++ + ++\|\|\|\|\|\|\|+\|+\|\|\|+\|\|\|\|\|\| | | |
| NOV17 | 26 KVFRVNVEDENHINIIRELATFI--QIDFWKPDSVTQIKPHSTVDFR | 70 | (SEQ ID NO:272) |
| | VpaediqavksfLeqsgIhYevlIeDVqelLeeqf<-* | | |
| | \|+\|\|\| +\|+++\|+\|++++\|+\|\|\|++++++ \| \|\| | | |
| NOV17 | 71 VKAEDTVTVENVLKQNELQYKVLISNLRNVVEAQF | 105 | |

Zn_carbOpept: domain 1 of 2, from 119 to 236: score 206.6, E=3.8e−58

| | | | |
|---|---|---|---|
| | *->YhnleeiyawlDllvsnfPdLvskvsiGksyeGRdlkvLKisdnpat | | (SEQ ID NO:273) |
| | \|+++\|+\|+\|\|++++++++\| \|+\|+++\|\|+++\|\|\| +++\|\|+++ | | |
| NOV17 | 119 YNKWETIEAWTQQVATENPALISRSVIGTTFEGRAIYLLKVGKA-- | 162 | (SEQ ID NO:274) |
| | genePevfavagWiHAREwvtsAtllwllkelvanYgsDktitklldgld | | |
| | \|+\|+\|++\|+++\| +\|\|\|\|\|+++\|+++\|+++\|+\|++\|\|++++ \|+\|\|\|+\|\| | | |
| NOV17 | 163 GQNKPAIFMECG-FHAREWISPAFCQWFVREAVRTYGREIQVTELLDKLD | 211 | |
| | lfyilpvfNpDGyaYsittdSyRmWRKt<-* | | |
| | \|\|+\|\|\| \|+\|\|\|+\|++\|+ \| \|\|\|\| | | |
| NOV17 | 212 -FYVLPVLNIDGYIYTWTKS--RFWRKT | 236 | |

Zn_carbOpept: domain 2 of 2, from 242 to 332: score 149.5, E=6e−41

| | | | |
|---|---|---|---|
| | *->llyPYgydynlnpdandldelsdlkiaadalsarhgtyYtlglpgss | | (SEQ ID NO:275) |
| | ++\|\|\|+\| \|+\| +++ +\|++\|+ \|+ ++ \|+++\|\|\|+\|\|+\| \|\|++ | | |
| NOV17 | 242 WIYPYSYAYKLGENNAELNALA--KATVKELASLHGTKYTYG-PGAT | 285 | (SEQ ID NO:276) |
| | tIYpasAGGsdDwaydvgiikyaftfElrpdtgsyGnPCFllPeeqIipt | | |
| | \|\|\|\|\|+ \|\|\|\|\|\|\|\|\|+\| \| \|+\|\|\|\|\|\| \|\|\|+\|\| \|\|\|\|\|+\|\|+ \| | | |
| NOV17 | 286 TIYPAA-GGSDDWAYDQG-IRYSFTFELR-DTGRYG---FLLPESQIRAT | 329 | |
| | gsee<-* | | |
| | ++ \| | | |
| NOV17 | 330 CE-E 332 | | |

The carboxypeptidase A family (M14) can be divided into two subfamilies: carboxypeptidase H (regulatory) and carboxypeptidase A (digestive). Members of the H family have longer C-termini than those of family A, and carboxypeptidase M (a member of the H family) is bound to the membrane by a glycosylphosphatidylinositol anchor, unlike the majority of the M14 family, which are soluble. See, InterPro IPR000834.

The zinc ligands have been determined as two histidines and a glutamate, and the catalytic residue has been identified as a C-terminal glutamate, but these do not form the characteristic metalloprotease HEXXH motif. Members of the carboxypeptidase A family are synthesised as inactive molecules with propeptides that must be cleaved to activate the enzyme. Structural studies of carboxypeptidases A and B reveal the propeptide to exist as a globular domain, followed by an extended alpha-helix; this shields the catalytic site, without specifically binding to it, while the substrate-binding site is blocked by making specific contacts.

The domain information indicates that the NOV17 sequence of the invention has properties similar to those of other proteins known to contain this/these domain(s) and similar to the properties of these domains.

A human pancreas-specific protein (PASP), previously characterized as a serum marker for acute pancreatitis and pancreatic graft rejection, has been identified as pancreatic procarboxypeptidase B (PCPB). cDNAs encoding PASPIPCPB were isolated from a human pancreas cDNA library using a combination of nucleic acid hybridization screening and immunoscreening with antisera raised against native PASP. The deduced amino acid sequence of PASPIPCPB cDNA predicts the translation of a 416-amino acid preproenzyme with a 15-amino acid signal/leader peptide and a 95-amino acid activation peptide. The proenzyme portion of this protein has 76% identity with rat PCPB and 84% identity with bovine carboxypeptidase B. DNA and RNA blot analyses indicate that human PCPB mRNA (1,400 nucleotides) is transcribed from a single locus in the human genome in a tissue-specific fashion. N-terminal sequencing of native PASP and the specific immunoreactivity of bacterially expressed PASP/PCPB with native PASP antibodies confirm the identification of PASP as human pancreatic PCPB. PMID: 1370825

In contrast to procarboxypeptidase B which has always been reported to be secreted by the pancreas as a monomer, procarboxypeptidase A occurs as a monomer and/or associated to one or two functionally different proteins, depending on the species. Recent studies showed that, in the human pancreatic secretion, procarboxypeptidase A is mainly secreted as a 44 kDa protein involved in at least three different binary complexes. As previously reported, two of these complexes associated procarhoxypeptidase A to either a glycosylated truncated protease E or zymogen E. In this paper, we identified proelastase 2 as the partner of procarboxypeptidase A in the third complex, thus reporting for the first time the occurrence of a proelastase 2/procarboxypeptidase A binary complex in vertebrates. Moreover, from N-terminal sequence analyses, the 44 kDa procarboxypeptidase A involved in these complexes was identified as being of the Al type. Only one type of procarboxypeptidase B, the B1 type, has been detected in the analyzed pancreatic juices, thus emphasizing the previously observed genetic differences between individuals. PMID: 2307232

Carboxypeptidase B1 is a highly tissue-specific protein and is a useful serum marker for acute pancreatitis and dysfunction of pancreatic transplants. It is not elevated in pancreatic carcinoma. The protein, referred to as pancreas-specific protein (PSAP) by Yamamoto et al. (1992), has a molecular mass of 44,500 Da and constitutes about 2% of total pancreatic cytosolic proteins. A computer search of protein sequence data using the first 25 amino acids from the N-terminal end suggested that PASP is pancreatic procarboxypeptidase B. Yamamoto et al. (1992) isolated a cDNA for PASP/PCPB and demonstrated that the deduced amino acid sequence represented a 416-amino acid preproenzyme with a 15-amino acid signal/leader peptide and a 95-amino acid activation peptide. RNA blot analyses indicated that the human PCPB mRNA, with 1,400 nucleotides, is transcribed from a single locus in the human genome in a tissue-specific fashion. See Yamamoto, et al., J. Biol. Chem. 267: 2575-2581, 1992. PubMed ID: 1370825.

The protein similarity information, expression pattern, cellular localization, and map location for the NOV17 protein and nucleic acid disclosed herein suggest that this Carboxypeptidase B, Pancreatic-like protein may have important structural and/or physiological functions characteristic of the Carboxypeptidase B, Pancreatic family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV17 nucleic acids and proteins of the invention have applications in the diagnosis and/or treatment of various diseases and disorders. For example, the compositions of the present invention will have efficacy for the treatment of patients suffering from: diabetes, Von Hippel-Lindau (VHL) syndrome, pancreatitis, obesity, ulcers, digestive disorders as well as other diseases, disorders and conditions.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV17 protein has multiple hydrophilic regions, each of which can be used as an immunogen. In one embodiment, a contemplated NOV17 epitope is from about amino acids 25 to 45. In another embodiment, a contemplated NOV17 epitope is from about amino acids 60 to 80. In other specific embodiments, contemplated NOV17 epitopes are from about amino acids 80 to 85, 110 to 130, 160 to 162, 170 to 172, 180 to 202, 240 to 260, 265 to 268, 290 to 305 and 310 to 320.

NOV18

One NOVX protein of the invention, referred to herein as NOV18, includes two Ribosomal Protein L29-like proteins. The disclosed proteins have been named NOV18a and NOV18b.

NOV18a

A disclosed NOV18a (designated CuraGen Acc. No. CG57113-01), which encodes a novel Ribosomal Protein L29-like protein and includes the 649 nucleotide sequence (SEQ ID NO:55) is shown in Table 18A. An open reading frame for the mature protein was identified beginning with an ATG initiation codon at nucleotides 43-45 and ending with a TAG stop codon at nucleotides 526-528. Putative untranslated regions are underlined in Table 18A, and the start and stop codons are in bold letters.

TABLE 18A
NOV18a Nucleotide Sequence

(SEQ ID NO:55)

ACTCACTATAGGGCTCGAGCGGCCGCCCGGGCAGGTGCAGACATGGCCAAGTCCAAGAACCACACCAC
ACACAACCAGTCCCGAAAATGGCACAGAAATGGTATCAAGAAACCCCGATCACAAAGATACGAATCTC
TTAAGGGGGTGGACCCCAAGTTCCTGAGGAACATGCGCTTTGCCAAGAAGCACAACAAAAAGGGCCTA
AAGAAGATGCAGGCCAACAATGCCAAGGCCATGAGTGCACGTGCCGAGGCTATCAAGGCCCTCGTAAA
GCCCAAGGAGGTTAAGCCCAAGATCCCAAAGGGTGTCAGCCGCAAGCTCGATCGACTTGCCTACATTG
CCCACCCCAAGCTTGGGAAGCGTGCTCGTGCCCGTATTGCCAAGGGGCTCAGGCTGTGCCGGCCAAAG
GCCAAGGCCAAGGCCAAGGCCAAGGCCAAGGATCAAACCAAGGCCCAGGCTGCAGCCCCAGCTTCAGT
TCCAGCTCAGGCTCCCAAACGTACCCAGGCCCCTACAAAGGCTTCAGAGTAGATATCTCTGCCAACAT
GAGGACAGAAGGACTGGTGCGACCCCCCACCCCCGCCCCTGGGCTACCATCTGCATGGGGCTGGGGTC
CTCCTGTGCTACTGGTACAAATAAACCTGAGGCAGGA

The disclosed NOV18a nucleic acid sequence maps to chromosome 3q29-qter and has 620 of 630 bases (98%) identical to a gb:GENBANK-ID:HSU10248|acc:U10248.1 mRNA from Homo sapiens (Human ribosomal protein L29 (humrpl29) mRNA, complete cds) (E=4.7e⁻¹²⁹).

A disclosed NOV18a polypeptide (SEQ ID NO:56) is 161 amino acid residues in length and is presented using the one-letter amino acid code in Table 18B. The SignalP, Psort and/or Hydropathy results predict that NOV18a does not have a signal peptide and is likely to be localized to the nucleus with a certainty of 0.9840. In alternative embodiments, a NOV18a polypeptide is located to the mitochondrial matrix space with a certainty of 0.1000 or the lysosome (lumen) with a certainty of 0.1000.

TABLE 18B
Encoded NOV18 a Protein Sequence

(SEQ ID NO:56)

MAKSKNHTTHNQSRKWHRNGIKKPRSQRYESLKGVDPKFLRNMRFAKKHNKKGLKKMQANNAKAMSARAEAIKALVK
PKEVKPKIPKGVSRKLDRLAYIAHPKLGKRARARIAKGLRLCRPKAKAKAKAKAKDQTKAQAAAPASVPAQAPKRTQ
APTKASE

The NOV18a amino acid sequence was found to have 159 of 161 amino acid residues (98%) identical to, and 159 of 161 amino acid residues (98%) similar to, the 159 amino acid residue ptnr:pir-id:S65784 protein from human (ribosomal protein L29, cytosolic) (E=2.56⁻⁷⁹).

NOV18a is expressed in at least the following tissues: adrenal gland, bone marrow, brain—amygdala, brain—cerebellum, brain—hippocampus, brain—substantia nigra, brain—thalamus, brain-whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus, Adipose, Amnion, Aorta, Appendix, Artery, Ascending Colon, Bone, Bronchus, Brown adipose, Buccal mucosa, Cartilage, Cerebral Medulla/Cerebral white matter, Cervix, Chorionic Villus, Colon, Coronary Artery, Dermis, Epidermis, Foreskin, Frontal Lobe, Gall Bladder, Gastro-intestinal/Digestive System, Hair Follicles, Hypothalamus, Kidney Cortex, Larynx, Left cerebellum, Liver, Lung, Lung Pleura, Lymph node, Lymphoid tissue, Muscle, Ovary, Oviduct/Uterine Tube/Fallopian tube, Parathyroid Gland, Parietal Lobe, Parotid Salivary glands, Peripheral Blood, Pineal Gland, Pituitary Gland, Respiratory Bronchiole, Retina, Right Cerebellum, Skin, Spongy Bone/Cancellous bone, Synovium/Synovial membrane, Temporal Lobe, Thymus, TonsilsUmbilical Vein, Urinary Bladder, Vein, Vulva, White adipose, and Whole Organism. Expression information was derived from the tissue sources of the sequences that were included in the derivation of the sequence of NOV18a.

NOV18b

A disclosed NOV18b (designated CuraGen Acc. No. CG57113-02), which includes the 580 nucleotide sequence (SEQ ID NO:57) shown in Table 18C. An open reading frame for the mature protein was identified beginning with an ATG codon at nucleotides 54-56 and ending with a TAG codon at nucleotides 537-539. The start and stop codons of the open reading frame are highlighted in bold type. Putative untranslated regions are underlined.

TABLE 18C
NOV18 b Nucleotide Sequence

(SEQ ID NO:57)

ACTCACTATAGGGCTCGAGCGGCGCTTCGGGAGCCGCGGCTTATGGTGCAGACATGGCCAAGTCCAAGAACCACA
CCACACACAACCAGTCCCGAAAATGGCACAGAAATGGTATCAAGAAACCCCGATCACAAAGATACGAATCTCTTA
AGGGGGTGGACCCCAAGTTCCTGAGGAACATGCGCTTTGCCAAGAAGCACAACAAAAAGGGCCTAAAGAAGATGC
AGGCCAACAATGCCAAGGCCATGAGTGCACGTGCCGAGGCTATCAAGGCCCTCGTAAAGCCCAAGGAGGTTAAGC
CCAAGATCCCAAAGGGTGTCAGCCGCAAGCTCGATCGACTTGCCTACATTGCCCACCCCAAGCTTGGGAAGCGTG
CTCGTGCCCGTATTGCCAAGGGGCTCAGGCTGTGCCGGCCAAAGGCCAAGGCCAAGGCCAAAGCCAAGGCCAAGG
ATCAAACCAAGGCCCAGGCTGCAGCCCCAGCTTCAGTTCCAGCTCAGGCTCCCAAACGTACCCAGGCCCCTACAA
AGGCTTCAGAGTAGATATCTCTGCCAACATGAGGACAGAAAGACTGGTGCGACCC

The disclosed NOV18b nucleic acid sequence maps to chromosome 3q29-qter and has 548 of 555 bases (98%) identical to a gb:GENBANK-ID:HSU10248|acc:U10248.1 mRNA from Homo sapiens (Human ribosomal protein L29 (humrp129) mRNA, complete cds) (E=1.2e⁻¹¹⁴).

The NOV18b polypeptide (SEQ ID NO:58) is 161 amino acid residues in length and is presented using the one-letter amino acid code in Table 18D. The SignalP, Psort and/or Hydropathy results predict that NOV18b has a signal peptide and is likely to be localized to the nucleus with a certainty of 0.9840. In alternative embodiments, a NOV18b polypeptide is located to the mitochondrial matrix space with a certainty of 0.1000 or the lysosome (lumen) with a certainty of 0.4600.

TABLE 18D
Encoded NOV18b Protein Sequence

(SEQ ID NO:58)

MAKSKNHTTHNQSRKWHRNGIKKPRSQRYESLKGVDPKFLRNMRFAKKHNKKGLKKMQANNAKAMSARAEAIKALV
KPKEVKPKIPKGVSRKLDRLAYIAHPKLGKRARARIAKGLRLCRPKAKAKAKAKAKDQTKAQAAAPASVPAQAPKR
TQAPTKASE

The NOV18b amino acid sequence was found to have 159 of 161 amino acid residues (98%) identical to, and 159 of 161 amino acid residues (98%) similar to, the 159 amino acid residue ptnr:pir-id:S65784 protein from human (ribosomal protein L29, cytosolic) (E=2.7e⁻⁷⁹).

NOV18b is expressed in at least the following tissues: adrenal gland, bone marrow, brain—amygdala, brain—cerebellum, brain—hippocampus, brain—substantia nigra, brain—thalamus, brain-whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus, Adipose, Amnion, Aorta, Appendix, Artery, Ascending Colon, Bone, Bronchus, Brown adipose, Buccal mucosa, Cartilage, Cerebral Medulla/Cerebral white matter, Cervix, Chronic Villus, Colon, Coronary Artery, Dermis, Epidermis, Foreskin, Frontal Lobe, Gall Bladder, Gastrointestinal/Digestive System, Hair Follicles, Hypothalamus, Kidney Cortex, Larynx Left cerebellum, Liver, Lung, Lung Pleura, Lymph node, Lymphoid tissue, Muscle, Ovary, Oviduct/Uterine Tube/Fallopian tube, Parathyroid Gland, Parietal Lobe, Parotid Salivary glands, Peripheral Blood, Pineal Gland, Pituitary Gland, Respiratory Bronchiole, Retina, Right Cerebellum, Skin, Spongy Bone/Cancellous bone, Synovium/Synovial membrane, Temporal Lobe, Thymus, TonsilsUmbilical Vein, Urinary Bladder, Vein, Vulva, White adipose, and Whole Organism. Expression information was derived from the tissue sources of the sequences that were included in the derivation of the sequence of NOV18b.

The sequence is predicted to be exxpressed in heart because of the expression pattern of (GENBANK-ID: gb:GENBANK-ID:HSU10248|acc:U10248.1) a closely related Human ribosomal protein L29 (humrpl29) mRNA, complete cds homolog in species Homo sapiens.

The nucleic acids for NOV18a and NOV18b are very closely homologous as is shown in the alignment in Table 18E. The disclosed NOV18a and NOV18b proteins are identical.

[image]

[image]

Homologies to any of the above NOV18 proteins will be shared by the other NOV18 proteins insofar as they are homologous to each other as shown above. Any reference to NOV18 is assumed to refer to both of the NOV18 proteins in general, unless otherwise noted.

NOV18 also has homology to the amino acid sequences shown in the BLASTP data listed in Table 18F.

TABLE 18F
BLAST results for NOV18

| Gene Index/ | | Length | Identity | Positives | |
|---|---|---|---|---|---|
| Identifier | Protein/Organism | (aa) | (%) | (%) | Expect |
| gi\|4506629\|ref\| | ribosomal protein | 159 | 159/161 | 159/161 | 2e−39 |
| NP_000983.1\|(NM_000992) | L29; 60S ribosomal | | (98%) | (98%) | |
| | protein L29; | | | | |
| | heparin/heparan | | | | |
| | sulfate- | | | | |
| | interacting | | | | |
| | protein; HP/HS- | | | | |
| | interacting | | | | |
| | protein; | | | | |
| | heparin/heparan | | | | |
| | sulfate-binding | | | | |
| | protein; cell | | | | |
| | surface heparin- | | | | |
| | binding protein | | | | |
| | HIP [Homo sapiens] | | | | |
| gi\|13642818\|ref\| | hypothetical | 157 | 152/161 | 153/161 | 2e−38 |
| XP_018182.1\|(XM_018182) | protein XP_018182 | | (94%) | (94%) | |
| | [Homo sapiens] | | | | |
| gi\|13648543\|ref\| | hypothetical | 155 | 151/161 | 151/161 | 4e−38 |
| XP_017364.1\|(XM_017364) | protein XP_017364 | | (93%) | (93%) | |
| | [Homo sapiens] | | | | |
| gi\|1082766\|pir\|\| | ribosomal protein | 159 | 157/161 | 157/161 | 6e−37 |
| S54204 | L29 - human | | (97%) | (97%) | |
| gi\|17456336\|ref\| | similar to | 189 | 128/158 | 138/158 | 7e−37 |
| XP_063630.1\|(XM_063630) | ribosomal protein | | (81%) | (87%) | |
| | L29; | | | | |
| | heparin/heparan | | | | |
| | sulfate | | | | |
| | interacting | | | | |
| | protein (H. | | | | |
| | sapiens) [Homo | | | | |
| | sapiens] | | | | |

The homology of these sequences is shown graphically in the ClustalW analysis shown in Table 18G.

[image]

[image]

Table 18H lists the domain description from DOMAIN analysis results against NOV18. This indicates that the NOV18 sequence has properties similar to those of other proteins known to contain these domains.

TABLE 18H
Domain Analysis of NOV18

gnl|Pfam|pfam01779, Ribosomal_L29e, Ribosomal L29e protein family.
CD-Length = 40 residues, 100.0% aligned
Score = 48.1 bits (113), Expect = 4e−07
NOV18: 3 KSKNHTTHNQSRKWHRNGIKEPRSQRYESLKGVDPKFLRN 42 (SEQ ID NO:282)
|||||||||++|||||||||++||+||||||||||
Sbjct: 1 KSKNHTNHNQNKKAHRNGIKKPQKKRYLSLKGVDAKFRRN 40 (SEQ ID NO:283)

Ribosomal protein L29e forms part of the 60S ribosomal subunit. This family is found in eukaryotes. There are there are 20 to 22 copies of the L29 gene in rat. Rat L29 is related to yeast ribosomal protein YL43. See InterPro IPR002673. Human ribosomal protein L29 has been shown to have the same nucleotide sequence as that of cell surface heparin/beparan sulfate-binding protein (Genomics Nov. 15, 1997;46(1):148-51). Heparan sulfate proteoglycans and their corresponding binding sites have been suggested to play an important role during the initial attachment of murine blastocysts to uterine epithelium and human trophoblastic cell lines to uterine epithelial cell lines (J Biol Chem May 17, 1996;271 (20): 11817-23). Heparin/heparan sulfate interacting protein (HIP) has been shown to be up-regulated in colorectal carcinoma. HIP is a candidate marker of abnormal cell growth in the colon and a prognostic marker for colorectal carcinoma. (Cancer Res Jun. 15, 1999;59(12):2989-94). Therefore it is likely that this novel ribosomal protein L29-like protein may play roles in blastocyst attachment and in tumorigenesis.

The protein synthesis reactions require a complex catalytic machinery to guide them. The growing end of the polypeptide chain, for example, must be kept in register with the mRNA molecule to ensure that each successive codon in the mRNA engages precisely with the anticodon of a tRNA molecule and does not slip by one nucleotide, thereby changing the reading frame. This precise movement and the other events in protein synthesis are catalyzed by ribosomes, which are large complexes of RNA and protein molecules. Eucaryotic and procaryotic ribosomes are very similar in design and function. Both are composed of one large and one small subunit that fit together to form a complex with a mass of several million daltons. The small subunit binds the mRNA and tRNAs, while the large subunit catalyzes peptide bond formation. More than half of the weight of a ribosome is RNA, and there is increasing evidence that the ribosomal RNA (rRNA) molecules play a central part in its catalytic activities. Ribosomes contain a large number of proteins, but many of these have been relatively poorly conserved in sequence during evolution.

During the large scale partial sequencing of human heart cDNA clones, a novel clone which is very similar to the rat ribosomal protein L29 in both DNA and amino acid sequences has been found. The cDNA encodes a protein with a deduced molecular weight of 17751 (159 aa). It shows 80.4% homology to protein L29 from the large ribosomal subunit of rat and is related to yeast YL43. The putative protein has been named human ribosomal protein L29 (hRPL29). hRPL29 has a large excess of basic residues over acidic ones. The large amount of charged residues makes the protein very hydrophilic and the protein has a deduced p1 of 12.16. Internal repeats have been characterized in many ribosomal proteins and a tandem repeat of KAKAKAKA (SEQ ID NO:284) was found to be unique to hRPL29. Northern analysis indicated that the mRNA that encodes human L29 is approx. 800 base pairs in length. An intron of hrpL29 has also been cloned and sequenced by polymerase chain reaction using human genomic DNA as the template.

By somatic cell hybrid analysis, radiation hybrid mapping, and fluorescence in situ hybridization, hRPL29 has been located on the telomeric region of the q arm of chromosome 3. hRPL29 is the most distal marker of the long arm of chromosome 3. Of the human ribosomal protein genes mapped, hRPL29 is the shortest distance from another ribosomal protein gene marker, hRPL35 a which has also been mapped to the 3q29-qter region. The human ribosomal protein L29 has been subsequently shown to have the same nucleotide sequence as that of cell surface heparin/heparan sulfate-binding protein, designated HP/HS interacting protein (HIP). Transfection of HIP full-length cDNA into N1H-3T3 cells demonstrates cell surface expression and a size similar to that of HIP expressed by human cells. Predicted amino acid sequence indicates that HIP lacks a membrane spanning region and has no consensus sites for glycosylation. Northern blot analysis detects a single transcript of 1.3 kilobases in both total RNA and poly(A+) RNA. Examination of human cell lines and normal tissues using both Northern blot and Western blot analyses reveals that HIP is expressed at different levels in a variety of human cell lines and normal tissues but absent in some cell lines and some cell types of normal tissues examined. Thus, members of the L29 family may be displayed on cell surfaces where they may participate in HP/HS binding events. Heparan sulfate proteoglycans and their corresponding binding sites have been suggested to play an important role during the initial attachment of murine blastocysts to uterine epithelium and human trophoblastic cell lines to uterine epithelial cell lines.

The protein similarity information, expression pattern, cellular localization, and map location for the protein and nucleic acid disclosed herein suggest that this ribosomal protein L29-like protein may have important structural and/or physiological functions characteristic of the ribosomal L29e proteins family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention may have efficacy for the treatment of patients suffering from cancer, especially colorectal carcinoma as well as other diseases, disorders and conditions.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV18 protein has multiple hydrophilic regions, each of which can be used as an immunogen. In one embodiment, a contemplated NOV18 epitope is from about amino acids 10 to 25. In another embodiment, a contemplated NOV18 epitope is from about amino acids 45 to 62. In other specific embodiments, contemplated NOV18 epitopes are from about amino acids 70 to 75, 78 to 82, 90 to 95, 110 to 112, 118 to 125 and 140 to 145

NOV19

A disclosed NOV19 (designated CuraGen Acc. No. CG57211-01), which encodes a novel Metalloproteinase-Disintegrin (ADAM30)-like protein and includes the 1143 nucleotide sequence (SEQ ID NO:59) is shown in Table 19A. An open reading frame for the mature protein was identified beginning with an ATG initiation codon at nucleotides 1-3 and ending with a TAA stop codon at nucleotides 1141-1143. The start and stop codons are in bold letters in Table 19A.

TABLE 19A
NOV19 Nucleotide Sequence

(SEQ ID NO:59)

ATGAGGTCAGTGCAGATCTTCCTCTCCCAATGCCGTTTGCTCCTTCTACTAGTTCCGACAATGCTCC
TTAAGTCTCTTGGCGAAGATGTAATTTTTCACCCTGAAGGGGAGTTTGACTCGTATGAAGTCACCAT
TCCTGAGAAGCTGAGCTTCCGGGGAGAGGTGCAGGGTGTGGTCAGTCCCGTGTCCTACCTACTGCAG
TTAAAAGGCAAGAAGCACGTCCTCCATTTGTGGCCCAAGAGACTTCTGTTGCCCCGACATCTGCGCG
TTTTCTCCTTCACAGAACATGGGGAACTGCTGGAGGATCATCCTTACATACCAAAGGACTGCAACTA
CATGGGCTCCGTGAAAGAGTCTCTGGACTCTAAAGCTACTATAAGCACATGCATGGGGGGTCTCCGA
GGTGTATTTAACATTGATGCCAAACATTACCAAATTGAGCCCCTCAAGGCCTCTCCCAGTTTTGAAC
ATGTCGTCTATCTCCTGAAGAAAGAGCAGTTTGGGAATCAGGCAGAAAATCTCATGTGCTGGGGCAC
AGGCTATCATCTATCCATGAAACCCATGGGAATACCTGACCTAGGTATGATAAATGATGGCACCTCC
TGTGGAGAAGGCCGGGTATGTTTTAAAAAAAATTGCGTCAATAGCTCAGTCCTGCAGTTTGACTGTT
TGCCTGAGAAATGCAATACCCGGGGTGTTTGCAACAACAGAAAAAGCTGCCACTGCATGTATGGGTG
GGCACCTCCATTCTGTGAGGAAGTGGGGTATGGAGGAAGCATTGACAGTGGGCCTCCAGGACTGCTC
AGAGGGGCGATTCCCTCGTCAATTTGGGTTGTGTCCATCATAATGTTTCGCCTTATTTTATTAATCC
TTTCAGTGGTTTTTGTGTTTTTCCGGCAAGTGATAGGAAACCACTTAAAACCCAAACAGGAAAAAAT
GCCACTATCCAAAGCAAAAACTGAACAGGAAGAATCTAAAACAAAAACTGTACAGGAAGAATCTAAA
ACAAAAACTGGACAGGAAGAATCTGAAGCAAAAACTGGACAGGAAGAATCTAAAGCAAAAACTGGAC
AGGAAGAATCTAAAGCAAACATTGAAAGTAAACGACCCAAAGCAAAGAGTGTCAAGAAACAAAAAAA
GTAA

The disclosed NOV19 nucleic acid sequence maps to chromosome 1 and has 635 of 636 bases (99%) identical to a gb:GENBANK-ID:AF171932.1 acc:AF171932.1 mRNA from Homo sapiens (Homo sapiens metallaproteinase-disintegrin (ADAM30) mRNA, complete cds) (E=1.5e⁻²⁵⁰).

A disclosed NOV19 polypeptide (SEQ ID NO:60) is 380 amino acid residues in length and is presented using the one-letter amino acid code in Table 19B. The SignalP, Psort and/or Hydropathy results predict that NOV19 has a signal peptide and is likely to be localized to the plasma membrane with a certainty of 04600. In alternative embodiments, a NOV19a polypeptide is located to the endoplasmic reticulum (membrane) with a certainty of 0.1000, the endoplasmic reticulum (lumen) with a certainty of 0.1000, or the outside of the cell with a certainty of 0.1000. The SignalP predicts a likely cleavage site for a NOV19 peptide between amino acid positions 27 and 28, i.e. at the sequence SLG-ED.

TABLE 19B
Encoded NOV19 Protein Sequence

(SEQ ID NO:60)

MRSVQIFLSQCRLLLLLVPTMLLKSLGEDVIFHPEGEFDSYEVTIPEKLSFRGEVQGVVSPVSYLLQLKGKKHVL
HLWPKRLLLPRHLRVFSFTEHGELLEDHPYIPKDCNYMGSVKESLDSKATISTCMGGLRGVFNIDAKHYQIEPLK
ASPSFEHVVYLLKKEQFGNQAENLMCWGTGYHLSMKPMGIPDLGMINDGTSCGEGRVCFKKNCVNSSVLQFDCLP
EKCNTRGVCNNRKSCHCMYGWAPPFCEEVGYGGSIDSGPPGLLRGAIPSSIWVVSIIMFRLILLILSVVFVFFRQ
VIGNHLKPKQEKMPLSKAKTEQEESKTKTVQEESKTKTGQEESEAKTGQEESKAKTGQEESKANIESKRPKAKSV
KKQKK

The NOV19 amino acid sequence was found to have 210 of 211 amino acid residues (99%) identical to, and 211 of 211 amino acid residues (100%) similar to, the 790 amino acid residue ptnr:SPTREMBL-ACC:Q9UKF2 protein from Homo sapiens (Human) (METALLAPROTEINASE-DISINTEGRIN) (E=2.3e⁻²⁰⁵).

NOV19 expressed in at least the following tissues: Adrenal Gland/Suprarenal gland, Prostate, Testis, and Whole Organism. Expression information was derived from the tissue sources of the sequences that were included in the derivation of the sequence of CuraGen Acc. No. CG57211-01. The sequence is predicted to be expressed in testis because of the expression pattern of (GENBANK-ID: gb:GENBANK-ID:AF171932|acc:AF171932.1), a closely related Homo sapiens metallaproteinase-disintegrin (ADAM30) mRNA, complete cds homolog in species Homo sapiens.

Homologies to any of the above NOV19 proteins will be shared by the other NOV19 proteins insofar as they are homologous to each other as shown above. Any reference to NOV19 is assumed to refer to both of the NOV19 proteins in general, unless otherwise noted.

Possible small nucleotide polymorphisms (SNPs) found for NOV19 are listed in Table 19C.

TABLE 19C
SNPs

| | Nucleotide | | Amino Acid | |
|---|---|---|---|---|
| Variant | Position | Base Change | Position | Base Change |
| 13376670 | 166 | C > T | 56 | Gln > End |
| 13376669 | 167 | A > G | 56 | Gln > Arg |
| 13376668 | 353 | A > G | 118 | Glu > Gly |
| 13376667 | 440 | A > G | 147 | Glu > Gly |
| 13376662 | 701 | G > A | 234 | Cys > Tyr |
| 13376661 | 736 | T > C | 246 | Trp > Arg |
| 13376660 | 979 | A > G | 327 | Thr > Ala |
| 13376659 | 989 | T > A | 330 | Val > Glu |

NOV19 also has homology to the amino acid sequences shown in the BLASTP data listed in Table 19D.

TABLE 19D
BLAST results for NOV19

| Gene Index/ | | Length | Identity | Positives | |
|---|---|---|---|---|---|
| Identifier | Protein/Organism | (aa) | (%) | (%) | Expect |
| gi\|11497609\|ref\| | a disintegrin and | 790 | 200/201 | 201/201 | e−118 |
| NP_068566.1\|(NM_021794) | metalloproteinase | | (99%) | (99%) | |
| | domain 30, | | | | |
| | isoform 1 | | | | |
| | preproprotein | | | | |
| | [Homo sapiens] | | | | |
| gi\|9966785\|ref\| | a disintegrin and | 781 | 191/201 | 191/201 | e−111 |
| NP_065067.1\|(NM_020334) | metalloproteinase | | (95%) | (95%) | |
| | domain 30, isoform | | | | |
| | 2 preproprotein | | | | |
| | [Homo sapiens] | | | | |
| gi\|9966766\|ref\| | a disintegrin and | 729 | 68/142 | 87/142 | 2e−31 |
| NP_065063.1\|(NM_020330) | metalloprotease | | (47%) | (60%) | |
| | domain 21; a | | | | |
| | disintegrin and | | | | |
| | metalloprotease | | | | |
| | domain (ADAM) 21 | | | | |
| | [Mus musculus] | | | | |
| gi\|14749466\|ref\| | a disintegrin and | 722 | 64/137 | 82/137 | 2e−31 |
| XP_016158.2\|(XM_016158) | metalloproteinase | | (46%) | (59%) | |
| | domain 21 | | | | |
| | preproprotein | | | | |
| | [Homo sapiens] | | | | |
| gi\|11497040\|ref\| | a disintegrin and | 722 | 64/137 | 82/137 | 2e−31 |
| NP_003804.1\|(NM_003813) | metalloproteinase | | (46%) | (59%) | |
| | domain 21 | | | | |
| | preproprotein | | | | |
| | [Homo sapiens] | | | | |

The homology of these sequences is shown graphically in the ClustalW analysis shown in Table 19E.

[image]

[image]

[image]

Table 19F lists the domain description from DOMAIN analysis results against NOV19. This indicates that the NOV19 sequence has properties similar to those of other proteins known to contain these domains.

TABLE 19F
Domain Analysis of NOV19

gnl|Pfam|pfam01562, Pep_M12B_propep, Reprolysin family propeptide. This region
is the propeptide for members of peptidase family M12B. The propeptide contains
a sequence motif similar to the “cysteine switch” of the matrixins. This motif
is found at the C terminus of the alignment but is not well aligned.
CD-Length=117 residues, only 71.8% aligned
Score = 90.1 bits (222), Expect = 2e−19

| | |
|---|---|
| NOV19: 76 HLWPKRLLLPRHLRVFSFTEHGELLEDHPYIPKDCNYMGSVKESLDSKATISTCMGGLRG 135 | (SEQ ID NO:290) |
| \|\| \|\|\| \|+++\|\|++\|\|\| \| + +\| ++\|\|\| \|\|\|\| | |
| Sbjct: 1 HLEKNRSLLAPDFTVTTYDDDGTLVTEHPLIQDHCYYQGYVEGYPNSAVSLSTC-SGLRG 59 | (SEQ ID NO:291) |
| NOV19: 136 VFNIDAKHYQIEPLKASPSFEHVVY 160 | |
| + ++ \|\|\|\|\|++\| \|\|\|++\| | |
| Sbjct: 60 ILQLENLSYGIEPLESSDGFEHIIY 84 | |

gn1|Smart|smart00608, ACR, ADAM Cysteine-Rich Domain
CD-Length+32 139 residues, 29.5% aligned
Score = 55.5 bits (132), Expect = 6e−09

| | |
|---|---|
| NOV19: 173 NLMCWGTGYHLSMKPMGIPDLGMINDGTSCGEGRVCFKKNCVNS 216 | (SEQ ID NO:292) |
| \|+\|\| \|\|\| \|\|\|\|\|\|+\|\|\|\|\|\|+\|\| \|\|+ | |
| Sbjct: 99 GLVCWSLDYHLGSD---IPDLGMVKDGTKCGPGKVCINGQCVDV 139 | (SEQ ID NO:293) |

A sequence of about thirty to forty amino-acid residues long found in the sequence of epidermal growth factor (EGF) has been shown, to be present, in a more or less conserved form, in a large number of other, mostly animal proteins. The list of proteins currently known to contain one or more copies of an EGF-like pattern is large and varied. The functional significance of EGF domains in what appear to be unrelated proteins is not yet clear. However, a common feature is that these repeats are found in the extracellular domain of membrane-bound proteins or in proteins known to be secreted (exception: prostaglandin G/H synthase). The EGF domain includes six cysteine residues which have been shown (in EGF) to be involved in disulfide bonds. The main structure is a two-stranded beta-sheet followed by a loop to a C-terminal short two-stranded sheet. Subdomains between the conserved cysteines vary in length. See InterPro IPR000561: EGF.

This indicates that the sequence of the invention has properties similar to those of other proteins known to contain this/these domain(s) and similar to the properties of these domains.

ADAMs are a family of cell surface proteins with a domain structure composed of a signal sequence, a prodomain with a cysteine switch, a metalloproteinase-like domain, a disintegrin-like domain, a cysteine-rich domain, a transmembrane domain, and a C-terminal cytoplasmic domain. Members of this family have been implicated in a variety of biologic processes involving cell-cell and cell-matrix interactions, including fertilization, muscle development, and neurogenesis.

By searching a DNA sequence database, Cerretti et al. (1999) identified 2 ESTs representing the novel ADAMs ADAM29 (604778) and ADAM30. The ADAM30 EST encodes a polypeptide with sequence similarity to the cysteine-rich region of ADAM21 (603713). Cerretti et al. (1999) screened a human testis cDNA library with the ADAM30 EST and isolated cDNAs encoding 2 forms of ADAM30 that differ in the cytoplasmic domain. The first predicted ADAM30 protein has 790 amino acids and contains all of the domains characteristic of ADAMs. The metalloproteinase domain of ADAM30 has a consensus zinc-binding motif, suggesting that ADAM30 is proteolytically active. The second form of ADAM30, which the authors called ADAM30-beta, has a deletion of 9 amino acids in its cytoplasmic domain compared to the first form, resulting in a protein with 781 amino acids. Northern blot analysis of a variety of human tissues detected an approximately 3.0-kb ADAM30 transcript only in testis.

The protein similarity information, expression pattern, cellular localization, and map location for the NOV19 protein and nucleic acid disclosed herein suggest that this Metallaproteinase-disintegrin (ADAM30)-like protein may have important structural and/or physiological functions characteristic of the ADAM family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV19 nucleic acids and proteins of the invention have applications in the diagnosis and/or treatment of various diseases and disorders. For example, the compositions of the present invention will have efficacy for the treatment of patients suffering from: fertility problems, adrenoleukodystrophy, congenital adrenal hyperplasia as well as other diseases, disorders and conditions.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV19 protein has multiple hydrophilic regions, each of which can be used as an immunogen. In one embodiment, a contemplated NOV19 epitope is from about amino acids 40 to 50. In another embodiment, a contemplated NOV19 epitope is from about amino acids 60 to 65. In other specific embodiments, contemplated NOV19 epitopes are from about amino acids 90 to 120, 140 to 152, 160 to 190, 195 to 205, 220 to 245, 249 to 252 and 310 to 370.

NOV20

A disclosed NOV20 (designated CuraGen Acc. No. CG57222-01), which encodes a novel Bone Morphogenetic Protein-like protein and includes the 1207 nucleotide sequence (SEQ ID NO:61) is shown in Table 20A. An open reading frame for the mature protein was identified beginning with an ATG initiation codon at nucleotides 54-56 and ending with a TAA stop codon at nucleotides 1089-1091. Putative untranslated regions are underlined in Table 20A, and the start and stop codons are in bold letters.

TABLE 20A
NOV20 Nucleotide Sequence

(SEQ ID NO:61)

CCGCGGGACTCCGGCGTCCCCGCCCCCCAGTCCTCCCTCCCCTCCCCTCCAGCATGGTGCTCGCGGCC
CCGCTGCTGCTGGGCTTCCTGCTCCTCGCCCTGGAGCTGCGGCCCCGGGGGGAGGCGGCCGAGGGCCC
CGCGGCGGCGGCGGCGGCGGCGGCGGCGGCGGCAGCGGCGGGGGTCGGGGGGGAGCGCTCCAGCCGGC
CAGCCCCGTCCGTGGCGCCCGAGCCGGACGGCTGCCCCGTGTGCGTATGGCGGCAGCACAGCCGCGAG
CTGCGCCTAGAGAGCATCAAGTCGCAGATCTTGAGCAAACTGCGGCTCAAGGAGGCGCCCAACATCAG
CCGCGAGGTGGTGAAGCAGCTGCTGCCCAAGGCGCCGCCGCTGCAGCAGATCCTGGACCTACACGACT
TCCAGGGCGACGCGCTGCAGCCCGAGGACTTCCTGGAGGAGGACGAGTACCACGCCACCACCGAGACC
GTCATTAGCATGGCCCAGGAGACGGACCCAGCAGTACAGACAGATGGCAGCCCTCTCTGCTGCCATTT
TCACTTCAGCCCCAAGGTGATGTTCACAAAGAGCATCGACTTCAAGCAAGTGCTACACAGCTGGTTCC
GCCAGCCACAGAGCAACTGGGGCATCGAGATCAACGCCTTTGATCCCAGTGGCACAGACCTGGCTGTC
ACCTCCCTGGGGCCGGGAGCCGAGGGGCTGCATCCATTCATGGAGCTTCGAGTCCTAGAGAACACAAA
ACGTTCCCGGCGGAACCTGGGTCTGGACTGCGACGAGCACTCAAGCGAGTCCCGCTGCTGCCGATATC
CCCTCACAGTGGACTTTGAGGCTTTCGGCTGGGACTGGATCATCGCACCTAAGCGCTACAAGGCCAAC
TACTGCTCCGGCCAGTGCGAGTACATGTTCATGCAAAAATATCCGCATACCCATTTGGTGCAGCAGGC
CAATCCAAGAGGCTCTGCTGGGCCCTGTTGTACCCCCACCAAGATGTCCCCAATCAACATGCTCTACT
TCAATGACAAGCAGCAGATTATCTACGGCAAGATACCTGGCATGGTGGTGGATCGCTGTGGCTGCTCT
TAAGTGGGTCACTACAAGCTGCTGGAGCAAAGACTTGGTGGGTGGGTAACTTAACCTCTTCACAGAGG
ATAAAAAATGCTTGTGAGTATGACAGAAGGGAATAAACAGGCTTAAAGGGT

The disclosed NOV20 nucleic acid sequence maps to chromosome 12 and has 597 of 629 bases (94%) identical to a gb:GENBANK-ID:AF100907|acc:AF100907.1 mRNA from Homo sapiens (Homo sapiens bone morphogenetic protein 11 (BMP11) mRNA, complete cds) (E=2.3e⁻²³⁵).

A disclosed NOV20 polypeptide (SEQ ID NO:62) is 345 amino acid residues in length and is presented using the one-letter amino acid code in Table 20B. The SignalP, Psort and/or Hydropathy results predict that NOV20 has a signal peptide and is likely to be localized to the outside of the cell with a certainty of 0.8200. In alternative embodiments, a NOV20a polypeptide is located to the endoplasmic reticulum (membrane) with a certainty of 0.1000, the endoplasmic reticulum (lumen) with a certainty of 0.1000, or the microbody (peroxisome) with a certainty of 0.1000. The SignalP predicts a likely cleavage site for a NOV20 peptide between amino acid positions 24 and 25, Le. at the sequence GEA-AE.

TABLE 20B
Encoded NOV20 Protein Sequence

(SEQ ID NO:62)

MVLAAPLLLGFLLLALELRPRGEAAEGPAAAAAAAAAAAAAGVGGERSSRPAPSVAPEPDGCPVCVWRQHSRELRL
ESIKSQILSKLRLKEAPNISREVVKQLLPKAPPLQQILDLHDFQGDALQPEDFLEEDEYHATTETVISMAQETDPA
VQTDGSPLCCHFHFSPKVMFTKSIDFKQVLHSWFRQPQSNWGIEINAFDPSGTDLAVTSLGPGAEGLHPFMELRVL
ENTKRSRRNLGLDCDEHSSESRCCRYPLTVDFEAFGWDWIIAPKRYKANYCSGQCEYMFMQKYPHTHLVQQANPRG
SAGPCCTPTKMSPINMLYFNDKQQIIYGKIPGKIPGMVVDRCGCS

The NOV20 amino acid sequence was found to have 171 of 172 amino acid residues (99%) identical to, and 172 of 172 amino acid residues (100%) similar to, the 407 amino acid residue ptnr:SWISSNEW-ACC:O95390 protein from Homo sapiens (Human) (GROWTH/DIFFERENTIATION FACTOR-11 PRECURSOR (BONE MORPHOGENETIC=2.5e⁻¹⁸⁸).

NOV20 is expressed in at least the following tissues: muscle, neural and uterine cells. Expression information was derived from the tissue sources of the sequences that were included in the derivation of the sequence of NOV20.

Possible small nucleotide polymorphisms (SNPs) found for NOV20 are listed in Table 20C.

TABLE 20C
SNPs

| | Nucleotide | | Amino Acid | |
|---|---|---|---|---|
| Variant | Position | Base Change | Position | Base Change |
| 13377014 | 460 | A > G | 136 | His > Arg |
| 13374718 | 591 | C > T | 180 | Gln > End |
| 13377008 | 702 | G > A | 217 | Glu > Lys |
| 13377013 | 725 | G > A | NA | NA |
| 13377012 | 747 | A > G | 232 | Lys > Glu |
| 13377011 | 870 | C > T | 273 | Arg > Cys |
| 13377009 | 1013 | G > A | 320 | Met > Ile |
| 13377010 | 896 | C > T | NA | NA |

Homologies to any of the above NOV20 proteins will be shared by the other NOV20 proteins insofar as they are homologous to each other as shown above. Any reference to NOV20 is assumed to refer to all of the NOV20 proteins in general, unless otherwise noted.

NOV20 also has homology to the amino acid sequences shown in the BLASTP data listed in Table 20D.

TABLE 20D
BLAST results for NOV20

| Gene Index/ | | Length | Identity | Positives | |
|---|---|---|---|---|---|
| Identifier | Protein/Organism | (aa) | (%) | (%) | Expect |
| gi\|6649914\|gb\| | growth/differentia | 379 | 306/379 | 309/379 | e−162 |
| AAF21630.1\|AF028333_{—} | tion factor-11 | | (80%) | (80%) | |
| 1 (AF028333) | [Homo sapiens] | | | | |
| gi\|5031613\|ref\| | growth | 407 | 334/407 | 337/407 | e−158 |
| NP_005802.1\| | differentiation | | (82%) | (82%) | |
| (NM_005811) | factor 11; bone | | | | |
| | morphogenetic | | | | |
| | protein 11 [Homo | | | | |
| | sapiens] | | | | |
| gi\|13124273\|sp\| | GROWTH/DIFFERENTIA | 405 | 323/407 | 326/407 | e−155 |
| Q9Z1W4\|GDFB_{—} | TION FACTOR 11 | | (79%) | (79%) | |
| MOUSE | PRECURSOR (BONE | | | | |
| | MORPHOGENETIC | | | | |
| | PROTEIN 11) | | | | |
| gi\|6649923\|gb\| | growth/differentia | 405 | 322/407 | 325/407 | e−155 |
| AAF21633.1\| | tion factor-11; | | (79%) | (79%) | |
| (AF028337) | GDF-11 [Mus | | | | |
| | musculus] | | | | |
| gi\|13124255\|sp\| | Growth/differentia | 345 | 267/345 | 271/345 | e−146 |
| Q9Z217\|GDFB_RAT | tion factor 11 | | (77%) | (78%) | |
| | precursor (Bone | | | | |
| | morphogenetic | | | | |
| | protein 11) | | | | |

The homology of these sequences is shown graphically in the ClustalW analysis shown in Table 20B.

[image]

[image]

Table 20F lists the domain description from DOMAIN analysis results against NOV20. This indicates that the NOV20 sequence has properties similar to those of other proteins known to contain these domains.

TABLE 20F
Domain Analysis of NOV20

gnl|Smart|smart002O4, TGFB, Transforming growth factor-beta (TGF-beta) family;
Family members are active as disulphide-linked homo- or heterodimers. TGFB is a
multifunctional peptide that controls proliferation, differentiation, and other
functions in many cell types.
CD-Length = 102 residues, 100.0% aligned
Score = 131 bits (329), Expect = 7e−432

| NOV20: 251 CCRYPLTVDFEAFGWD-WIIAPKRYKANYCSGQCEYMFMQKYPHTH------LVQQANPR 303 | (SEQ ID NO:299X) |
|---|---|
| \| \|+ \| \|\|\|+ \|\|\| \|\|\|\|\|\| \| \| \|\| \|+\| + ++ \|+ \|\| +\| | |
| Sbjct: 1 CRRHDLYVDFKDLGWDDWIIAPKGYNAYYCEGECPFPLSERLNATNHAIVQSLVHALDPG 60 | (SEQ ID NO:300) |
| NOV20: 304 GSAGPCCTPTKMSPINMLYFNDKQQIIYGKIPGMVVDRCGCS 345 | |
| \|\|\| \|\|\|+\|\|++\|\|\|++\| ++ \| \|\|\|+ \|\|\| | |
| Sbjct: 61 AVPKPCCVPTKLSPLSMLYYDDDGNVVLRNYPNMVVEECGCR 102 | |
| gnl\|Pfam\|pfam00019, TGF-beta, Transforming growth factor beta like domain. | |
| CD-Length = 105 residues, 97.1% aligned | |
| Score = 103 bits (256), Expect = 2e−23 | |
| NOV20: 251 CCRYPLTVDFEAFGW-DWIIAPKRYKANYCSGQCEYMFMQKYPHTH------LVQQANPR 303 | (SEQ ID NO:299) |
| \| \| \|\|\| \|\| \|\|\|\|\|\|+ \| \|\|\|\|\|\| \| + ++ \|\|+ \|\|\| | |
| Sbjct: 4CRLRSLYVDFRDLGWGDWIIAPEGYIANYCSGSCPFPLRDDLNLSNHAILQTLVRLRNPR 63 | (SEQ ID NO:300) |
| NOV20: 304 GSAGPCCTPTKMSPINMLYFNDKQQIIYGKIPGMVVDRCGCS 345 | |
| \|\|\| \|\|\|+\|\|++\|\|\| +\| ++ \| \| \| \|\|\| | |
| Sbjct: 64 AVPQPCCVPTKLSPLSMLYLDDNSNVVLRLYPNMSVKECGCR 105 | |

gn1|Pfam|pfam00688, TGF_propeptide, TGF-beta propeptide. This propeptide is
known as latency associated peptide (LAP) in TGF-beta. LAP is a homodimer which
is disulfide linked to TGF-beta binding protein.
CD-Length = 227 residues, 46.3% aligned
Score = 48.1 bits (113), Expect = 8e−07

| | |
|---|---|
| NOV20: 62 CPVCVWRQHSRELRLESIKSQILSKLRLKEAPNISREVVKQLLPKAPPLQQILDLHDFQG 121 | (SEQ ID NO:302) |
| \| \|+ ++ \|\|\|+\|+ \|\|\|\|\|\| \|+ \| \|+\| + +\|\|\|++ | |
| Sbjct: 1 CRPLDLRRSQKQDRLEAIEGQILSKLGLRRRPRPSKE-------PMVVPEYMLDLYNALS 53 | (SEQ ID NO:303) |
| NOV20: 122 DALQ--PEDFLEEDEYHATTETVISMAQ-----ETDPAVQTDGSPLCCHFHF 166 | |
| + + \| +\| + + \|+\| ++ \| \| | |
| Sbjct: 54 ELEEGKVGRVPEISDYDGREAGRANTIRSFSHLESDDFEESTPESHRKRFRF 105 | |

The homology and domain information indicate that the sequence of the invention has properties similar to those of other proteins known to contain this/these domain(s) and similar to the properties of these domains.

Transforming growth factor-beta (TGF-beta) is a multifunctional peptide that controls proliferation, differentiation and other functions in many cell types. TGF-beta-1 is a peptide of 112 amino acid residues derived by proteolytic cleavage from the C-terminal of a precursor protein. See IPR001839. A number of proteins are known to be related to TGF-beta-1. Proteins from the TGF-beta family are only active as homo- or heterodimer; the two chains being linked by a single disulfide bond. From X-ray studies of TGF-beta-2, it is known that all the other cysteines are involved in intrachain disulfide bonds. As shown in the following schematic representation, there are four disulfide bonds in the TGF-betas and in inhibin beta chains, while the other members of this family lack the first bond.

```
                                                                                                interchain
                                                                                                |
          +------------------------------------------|+
          | ||
xxxxcxxxxxCcxxxxxxxxxxxxxxxxxxCxxCxxxxxxxxxxxxxxxxxxxCCxxxxxxxxxxxxxxxxxxxCxCx
| | | | |
+------+##################+--|----------------------------------------+ |
                                                        +------------------------------------------+
‘C’: conserved cysteine involved in a disulfide bond
```

The transforming growth factor beta, N-terminus (TGFb) domain is present in a variety of proteins which include the transforming growth factor beta, decapentaplegic proteins and bone morphogenetic proteins. Transforming growth factor beta is a multifunctional peptide that controls proliferation, differentiation and other functions in many cell types. The decapentaplegic protein acts as an extracellular morphogen responsible for the proper development of the embryonic dorsal hypoderm, for viability of larvae and for cell viability of the epithelial cells in the imaginal disks. Bone morphogenetic protein induces cartilage and bone formation and may be responsible for epithelial osteogenesis in some organisms. See IPR001111.

The bones that comprise the axial skeleton have distinct morphologic features characteristic of their positions along the anterior/posterior axis. McPherron et al. (1997) described a novel mouse TGF-beta family member, myostatin, encoded by the gene Mstn (601788), that has an essential role in regulating skeletal muscle mass. By low-stringency screening, McPherron et al. (1997) also identified a gene related to Mstn. The cloning of this gene, designated Gdf11 (also called Bmp11), was also reported by Gamer et al. (1999) and Nakashima et al. (1999). McPherron et al. (1999) showed that Gdf11, a transforming growth factor-beta (TGF-beta) superfamily member, has an important role in establishing the patterning of the axial skeleton. They found that during early mouse embryogenesis Gdf11 is expressed in the primitive streak and tail bud regions, which are sites where new mesodermal cells are generated. Homozygous mutant mice carrying a targeted deletion of Gdf11 exhibited anteriorly directed homeotic transformations throughout the axial skeleton and posterior displacement of the hindlimbs. The effect of the mutation was dose dependent, as Gdf11+/− mice had a milder phenotype than Gdf11−/− mice. Mutant embryos showed alterations in patterns of Hox (see 142950) gene expression, suggesting that Gdf11 acts upstream of the Hox genes. McPherron et al. (1999) interpreted their findings to indicate that Gdf11 is a secreted signal that acts globally to specify positional identity along the anterior/posterior axis. To their knowledge, Gdf11 was the first secreted protein to be discovered that functions globally to regulate anterior/posterior axial patterning. The homeotic transformations observed in Gdf11 mutant mice were more extensive than those seen either by genetic manipulation of presumed patterning genes or by administration of retinoic acid. The question was raised of whether Gdf11 and retinoic acid interact to regulate Hox gene expression and anterior/posterior patterning and whether Gdf11 regulates the patterning of tissues other than those studied by McPherron et al. (1999).

The protein similarity information, expression pattern, cellular localization, and map location for the NOV20 protein and nucleic acid disclosed herein suggest that this Bone Morphogenetic Protein 11-like protein may have important structural and/or physiological functions characteristic of the TGF-beta family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV20 nucleic acids and proteins of the invention have applications in the diagnosis and/or treatment of various diseases and disorders. For example, the compositions of the present invention will have efficacy for the treatment of patients suffering from: muscle wasting disease, a neuromuscular disorder, muscle atrophy, obesity or other adipocyte cell disorders, and aging as well as other diseases, disorders and conditions.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV20 protein has multiple hydrophilic regions, each of which can be used as an immunogen. In one embodiment, a contemplated NOV20 epitope is from about amino acids 55 to 57. In another embodiment, a contemplated NOV20 epitope is from about amino acids 60 to 62. In other specific embodiments, contemplated NOV20 epitopes are from about amino acids 67 to 70, 90 to 99, 110 to 112, 115 to 117, 130 to 145, 148 to 149, 150 to 152, 158 to 161, 180 to 200, 230 to 250, 260 to 310 and 320 to 325.

NOV21

One NOVX protein of the invention, referred to herein as NOV21, includes three Adrenomedullin Receptor-like proteins. The disclosed proteins have been named NOV21 a, NOV21b and NOV21c.

NOV21a

A disclosed NOV21a (designated CuraGen Acc. No. CG56477-01), which encodes a novel Adrenomedullin Receptor-like protein and includes the 1341 nucleotide sequence (SEQ ID NO:63) is shown in Table 21A. An open reading frame for the mature protein was identified beginning with an ATG initiation codon at nucleotides 51-53 and ending with a TGA stop codon at nucleotides 1413-1415.

TABLE 21A
NOV21a Nucleotide Sequence

(SEQ ID NO:63)

CAGCCTCCTCACAGCTCCCCATAGCCTGGACCTGCCGGCCCTCCCTCCAGGACCGAGGGGCTCCCAAGGGAAAC
TCAGGCGTGTGCTGGTCCCAATGTCAGTGAAACCCAGCTGGGGGCCTGGCCCCTCGGAGGGGGTCACCGCAGTG
CCTACCAGTGACCTTGGAGAGATCCACAACTGGACCGAGCTGCTTGACCTCTTCAACCACACTTTGTCTGAGTG
CCACGTGGAGCTCAGCCAGAGCACCAAGCGCGTGGTCCTCTTTGCCCTCTACCTGGCCATGTTTGTGGTTGGGC
TGGTGGAGAACCTCCTGGTGATATGCGTCAACTGGCGCGGCTCAGGCCGGGCAGGGCTGATGAACCTCTACATC
CTCAACATGGCCATCGCGGACCTGGGCATTGTCCTGTCTCTGCCCGTGTGGATGCTGGAGGTCACGCTGGACTA
CACCTGGCTCTGGGGCAGCTTCTCCTGCCGCTTCACTCACTACTTCTACTTTGTCAACATGTATAGCAGCATCT
TCTTCCTGGTGTGCCTCAGTGTCGACCGCTATGTCACCCTCACCAGCGCCTCCCCCTCCTGGCAGCGTTACCAG
CACCGAGTGCGGCGGGCCATGTGTGCAGGCATCTGGGTCCTCTCGGCCATCATCCCGCTGCCTGAGGTGGTCCA
CATCCAGCTGGTGGAGGGCCCTGAGCCCATGTGCCTCTTCATGGCACCTTTTGAAACGTACAGCACCTGGGCCC
TGGCGGTGGCCCTGTCCACCACCATCCTGGGCTTCCTGCTGCCCTTCCCTCTCATCACAGTCTTCAATGTGCTG
ACAGCCTGCCGGCTGCGGCAGCCAGGACAACCCAAGAGCCGGCGCCACTGCTTGCTGCTGTGCGCCTACGTGGC
CGTCTTTGTCATGTGCTGGCTGCCCTATCATGTGACCCTGCTGCTGCTCACACTGCATGGGACCCACATCTCCC
TCCACTGCCACCTGGTCCACCTGCTCTACTTCTTCTATGATGTCATTGACTGCTTCTCCATGCTGCACTGTGTC
ATCAACCCCATCCTTTACAACTTTCTCAGCCCACACTTCCGGGGCCGGCTCCTGAATGCTGTAGTCCATTACCT
TCCTAAGGACCAGACCAAGGCGGGCACATGCGCCTCCTCTTCCTCCTGTTCCACCCAGCATTCCATCATCATCA
CCAAGGGTGATAGCCAGCCTGCTGCAGCAGCCCCCCACCCTGAGCCAAGCCTGAGCTTTCAGGCACACCATTTG
CTTCCAAATACTTCCCCCATCTCTCCCACTCAGCCTCTTACACCCAGCTGAGGTACTAGAATTCAGCGGCCGCT
GAATTCTAG

The NOV21 polypeptide (SEQ ID NO:64) is 404 amino acid residues in length and is presented using the one-letter amino acid code in Table 21B.

TABLE 21B
Encoded NOV21a Protein Sequence

(SEQ ID NO:64)

MSVKPSWGPGPSEGVTAVPTSDLGEIHNWTELLDLFNHTLSECHVELSQSTKRVVLGALYLAMFVVGLVENLLVIC
VNWRGSGRAGLMNLYILNMAIADLGIVLSLPVWMLEVTLDYTWLWGSFSCRFTHYFYFVNMYSSIFFLVCLSVDRY
VTLTSASPSWQRYQHRVRRAMCAGIWVLSAIIPLPEVVHIQLVEGPEPMCLFMAPFETYSTWALAVALSTTILGFL
LPFPLITVFNVLTACRLRQPGQPKSRRHCLLLCAYVAVFVMCWLPYHVTLLLLTLHGTHISLHCHLVHLLYFFYDV
IDCFSMLHCVINPILYNFLSPHFRGRLLNAVVHYLPKDQTKAGTCASSSSCSTQHSIIITKGDSQPAAAAPHPEPS
LSFQAHHLLPNTSPISPTQPLTPS

Possible small nucleotide polymorphisms (SNPs) found for NOV21 are listed in Table 21C.

TABLE 21C
SNPs

| | Nucleotide | | Amino Acid | |
|---|---|---|---|---|
| Variant | Position | Base Change | Position | Base Change |
| 13377037 | 363 | T > C | 90 | Leu > Pro |
| 13377038 | 604 | G > A | 170 | Arg > Arg |
| 13377039 | 685 | C > T | 197 | Gly > Gly |
| 13377040 | 1139 | T > C | 349 | Cys > Arg |

NOV21b

A disclosed NOV21b (designated CuraGen Acc. No. CG56477-02), which encodes a novel Adrenomedullin Receptor-like protein and includes the 945 nucleotide sequence (SEQ ID NO:65) is shown in Table 21 b. An open reading frame for the mature protein was identified beginning with an ATG initiation codon at nucleotides 1-3 and ending with a TGA stop codon at nucleotides 943-945. The start and stop codons are in bold letters in Table 21D.

TABLE 21D
NOV21b Nucleotide Sequence

(SEQ ID NO:65)

ATGTCAGTGAAACCCAGCTGGGGGCCTGGCCCCTCGGAGGGGGTCACCGCAGTGCCTACCAGTGACCTTGGAGA
GATCCACAACTGGACCGAGCTGCTTGACCACCTCTTCAACCACACTTTGTCTGAGTGCCACGTGGAGCTCAGCC
AGAGCACCAAGCGCGTGGTCCTCTTTGCCCTCTACCTGGCCATGTTTGTGGTTGGGCTGGTGGAGAACCTCCTG
GTGATATGCGTCAACTGGCGCGGCTCAGGCCGGGCAGGGCTGATGAACCTCTACATCCTCAACATGGCCATCGC
GGACCTGGGCATTGTCCTGTCTCTGCCCGTGTGGATGCCGGAGGTCACGCTGGACTACACCTGGCTCTGGGGCA
GCTTCTCCTGCCGCTTCACTCACTACTTCTACTTTGTCAACATGTATAGCAGCATCTTCTTCCTGGTGTGCCTC
AGTGTCGACCGCTATGTCACCCTCACAGGACAACCCAAGAGCCGGCGCCACTGCCTGCTGCTGTGCGCCTACGT
GGCCGTCTTTGTCATGTGCTGGCTGCCCTATCATGTGACCCTGCTGCTGCTCACACTGCATGGGACCCACATCT
CCCTCCACTGCCACCTGGTCCACCTGCTCTACTTCTTCTATGATGTCATTGACTGCTTCTCCATGCTGCACTGT
GTCATCAACCCCATCCTTTACAACTTTCTCAGCCCACACTTCCGGGGCCGGCTCCTGAATGCTGTAGTCCATTA
CCTTCCTAAGGACCAGACCAAGGCGGGCACATGCGCCTCCTCTTCCTCCTGTTCCACCCAGCATTCCATCATCA
TCACCAAGGGTGATAGCCAGCCTGCTGCAGCAGCAGCCCCCCACCCTGAGCCAAGCCTGAGCTTTCAGGCACAC
CATTTGCTTCCAAATACTTCCCCCATCTCTCCCACTCAGCCTCTTACACCCAGCTGA

The disclosed NOV21b nucleic acid sequence maps to chromosome 12 and has 473 of 476 bases (99%) identical to a gb:GENBANK-ID:AR012140|acc:AR012140.1 mRNA from Unknown (Sequence 1 from patent U.S. Pat. No. 5,763,218) (E=3.3e⁻²⁰²).

A disclosed NOV21b polypeptide (SEQ ID NO:66) is 314 amino acid residues in length and is presented using the one-letter amino acid code in Table 21 E. The SignalP, Psort and/or Hydropathy results predict that NOV21b has a signal peptide and is likely to be localized to the plasma membrane with a certainty of 0.6000. In alternative embodiments, a NOV21b polypeptide is located to the Golgi body with a certainty of 0.4000, the endoplasmic reticulum (membrane) with a certainty of 0.3000 or the mitochondrial inner membrane with a certainty of 0.0300. The SignalP predicts a likely cleavage site for a NOV37 peptide between amino acid positions 17 and 18, i.e. at the sequence VTA-VP.

TABLE 21E
Encoded NOV21b Protein Sequence

| | |
|---|---|
| MSVKPSWGPGPSEGVTAVPTSDLGEIHNWTELLDHLFNHTLSECHVELSQSTKRVVLFALYLAMFVV | (SEQ ID NO:66) |
| GLVENLLVICVNWRGSGRAGLMNLYILNMAIADLGIVLSLPVWMPEVTLDYTWLWGSFSCRFTHYFY | |
| FVNMYSSIFFLVCLSVDRYVTLTGQPKSRRHCLLLCAYVAVFVMCWLPYHVTLLLLTLHGTHISLHC | |
| HLVHLLYFFYDVIDCFSMLHCVINPILYNFLSPHFRGRLLNAVVHYLPKDQTKAGTCASSSSCSTQH | |
| SIIITKGDSQPAAAAAPHPEPSLSFQAHHLLPNTSPISPTQPLTPS | |

The NOV21b amino acid sequence was found to have 156 of 157 amino acid residues (99%) identical to, and 156 of 157 amino acid residues (99%) similar to, the 404 amino acid residue ptnr:SWISSNEW-ACC:015218 protein from Homo sapiens (Human) (ADRENOMEDULLIN RECEPTOR (AM-R)) (E=1.4e⁻¹⁶⁸).

NOV21b is expressed in at least the following tissues: heart, skeletal muscle, liver, pancreas, stomach, spleen, lymph node, bone marrow, adrenal gland, and thyroid.

Expression information was derived from the tissue sources of the sequences that were included in the derivation of the sequence of NOV21b.

NOV21c

A disclosed NOV21c (designated CuraGen Acc. No. CG56477-03), which encodes a novel Adrenomedullin Receptor-like protein and includes the 965 nucleotide sequence (SEQ ID NO:67) is shown in Table 21F. An open reading frame for the mature protein was identified beginning with an ATG initiation codon at nucleotides 3-5 and ending with a TGA stop codon at nucleotides 963-965. Putative untranslated regions are underlined in Table 21F, and the start and stop codons are in bold letters.

TABLE 21F
NOV21c Nucleotide Sequence

(SEQ ID NO:67)

CGATGTCAGTGAAACCCAGCTGGGGGCCTGGCCCCTCGGAGGGGGTCACCGCAGTGCCTACCAGTGACCTTGGAGA
GATCCACAACTGGACCGAGCTGCTTGACCTCTTCAACCACACTTTGTCTGAGTGCCACGTGGAGCTCAGCCAGAGC
ACCAAGCGCGTGGTCCTCTTTGCCCTCTACCTGGCCATGTTTGTGGTTGGGCTGGTGGAGAACCTCCTGGTGATAT
GCGTCAACTGGCGCGGCTCAGGCCGGGCAGGGCTGATGAACCTCTACATCCTCAACATGGCCATCGCGGACCTGGG
CATTGTCCTGTCTCTGCCCGTGTGGATGCTGGAGGTCACGCTGGACTACACCTGGCTCTGGGGCAGCTTCTCCTGC
CGCTTCACTCACTACTTCTACTTTGTCAACATGTATAGCAGCATCTTCTTCCTGCTGCCCTTCCCTCTCATCACAG
TCTTCAATGTGCTGACAGCCTGCCGGCTGCGGCAGCCAGGACAACCCAAGAGCCGGCGCCACTGCCTGCTGCTGTG
CGCCTACGTGGCCGTCTTTGTCATGTGCTGGCTGCCCTATCATGTGACCCTGCTGCTGCTCACACTGCATGGGACC
CACATCTCCCTCCACTGCCACCTGGTCCACCTGCTCTACTTCTTCTATGATGTCATTGACTGCTTCTCCATGCTGC
ACTGTGTCATCAACCCCATCCTTTACAACTTTCTCAGCCCACACTTCCGGGGCCGGCTCCTGAATGCTGTAGTCCA
TTACCTTCCTAAGGACCAGACCAAGGGCGGGCACATGCGCCTCCTCTTCCTCCTGTTCCACCCAGCATTCCATCAT
CATCACCAAGGTGATAGCCAGCCTGCTGCAGCAGCCCCCCACCCTGAGCCAAGCCTGAGCTTTCAGGCACACCATT
TGCTTCCAAATACTTCCCCCATCTCTCCCACTCAGCCTCTTACACCCAGCTGA

The disclosed NOV21c nucleic acid sequence maps to chromosome 12 and has 549 of 559 bases (98%) identical to a gb:GENEBANK-ID:AR012140|acc:AR12140.1 mRNA from Unknown. (Sequence 1 from patent U.S. Pat. No. 5,763,218) (B=9.3e⁻¹¹⁵).

A disclosed NOV21c polypeptide (SEQ ID NO:58) is 320 amino acid residues in length and is presented using the one-letter amino acid code in Table 21 G. The SignalP, Psort and/or Hydropathy results predict that NOV21c has a signal peptide and is likely to be localized to the plasma membrane with a certainty of 0.6000. In alternative embodiments, a NOV21c polypeptide is located to the Golgi body with a certainty of 0.4000, the endoplasm ic reticulum (membrane) with a certainty of 0.3000, or the mitochondrial inner membrane with a certainty of 0300. The SignaIP predicts a likely cleavage site for a NOV21c peptide between amino acid positions 14 and 15, i.e. at the sequence SEG3-VT.

TABLE 21G
Encoded NOV21c Protein Sequence

(SEQ ID NO:58)

MSVKPSWGPGPSEGVTAVPTSDLGEIHNWTELLDLFNHTLSECHVELSQSTKRVVLFALYLAMFVVGLVENLLVI
CVNWRGSGRAGLMNLYILNMAIADLGIVLSLPVWMLEVTLDYTWLWGSFSCRFTHYFYFVNMYSSIFFLLPFPLI
TVFNVLTACRLRQPGQPKSRRHCLLLCAYVAVFVMCWLPYHVTLLLLTLHGTHISLHCHLVHLLYFFYDVIDCFS
MLHCVINPILYNFLSPHFRGRLLNAVVHYLPKDQTKGGHMRLLFLLFHPAFHHHHQGDSQPAAAAPHPEPSLSFQ
AHHLLPNTSPISPTQPLTPS

The NOV21c amino acid sequence was found to have 159 of 178 amino acid residues (89%) identical to, and 160 of 178 amino acid residues (89%) similar to, the 404 amino acid residue ptnr:SWISSNEW-ACC:O15218 protein from Homo sapiens (Human) (ADRENOMEDULLIN RECEPTOR (AM-R)) (E=7.1e⁻⁸⁴).

NOV21c is expressed in at least the following tissues: heart, skeletal muscle, liver, pancreas, stomach, spleen, lymph node, bone marrow, adrenal gland, and thyroid. Expression information was derived from the tissue sources of the sequences that were included in the derivation of the sequence of NOV21c.

Homologies to any of the above NOV21a, NOV21b and NOV21c proteins will be shared by the other NOV21 proteins insofar as they are homologous to each other as shown above. Any reference to NOV21 is assumed to refer to NOV21a, NOV21b and NOV21c proteins in general, unless otherwise noted.

NOV21a, NOV21b and NOV21c are very closely homologous as is shown in the amino acid alignment in Table 21H.

[image]

[image]

NOV21a also has homology to the amino acid sequences shown in the BLASTP data listed in Table 21I.

TABLE 21I
BLAST results for NOV21a

| Gene Index/ | | Length | Identity | Positives | |
|---|---|---|---|---|---|
| Identifier | Protein/Organism | (aa) | (%) | (%) | Expect |
| gi\|6005705\|ref\| | adrenomedullin | 404 | 404/404 | 404/404 | 0.0 |
| NP_009195.1\| | receptor; G- | | (100%) | (100%) | |
| (NM_007264) | protein-coupled | | | | |
| | receptor similar | | | | |
| | to the | | | | |
| | adrenomedullin | | | | |
| | receptor [Homo | | | | |
| | sapiens] | | | | |
| gi\|6680654\|ref\| | adrenomedullin | 395 | 278/376 | 317/376 | e−148 |
| NP_031438.1\| | receptor [Mus | | (73%) | (83%) | |
| (NM_007412) | musculus] | | | | |
| gi\|16757998\|ref\| | adrenomedullin | 398 | 287/384 | 327/394 | e−145 |
| NP_445754.1\| | receptor [Rattus | | (72%) | (82%) | |
| (NM_053302) | norvegicus] | | | | |
| gi\|543446\|pir\|\| | probable G | 395 | 285/381 | 324/381 | e−143 |
| S40685 | protein-coupled | | (74%) | (84%) | |
| | receptor G10d - | | | | |
| | rat | | | | |
| gi\|12643978\|sp\| | ADRENOMEDULLIN | 395 | 282/380 | 321/380 | e−142 |
| P31392\|ADMR_{—} | RECEPTOR (AM-R) | | (74%) | (84%) | |
| RAT | (G10D) (NOW) | | | | |

The homology of these sequences is shown graphically in the ClustalW analysis shown in Table 21J.

[image]

[image]

Tables 21K and 21L list the domain description from DOMAIN analysis results against NOV21. This indicates that the NOV21 sequence has properties similar to those of other proteins known to contain these domains.

TABLE 21K
Domain Analysis of NOV21c

hmmpfam - search a single seq against 11MM database
HMM file: pfamHMMs
Scores for sequence family classification (score includes all domains):

| Model | Description | Score | E-value | N |
|---|---|---|---|---|
| 7tm_1 | 7 transmembrane receptor (rhodopsin family) 157.3 | 8e−49 | 2 | |

Parsed for domains:

| | | | | | | | |
|---|---|---|---|---|---|---|---|
| Model | Domain | seq seq | hmm | hmm | score | E-value | |
| | | from to | from to | | | | |
| 7tm_1 | 1/2 | 70 | 142 .. | 1 | 75 [. | 74.6 | 8.1e−23 |
| 7tm_1 | 2/2 | 143 | 236 .. | 173 | 259 .] | 86.7 | 1.3e−26 |

Alignments of top-scoring domains:
7tm_1: domain 1 of 2, from 70 to 142: score 74.6, E = 8.1e-23

| | | | | |
|---|---|---|---|---|
| | | *->GNlLVilvilrtkklr.tptnifilNLAvADLLflltlppwalyylv | | |
| | | +\|\|\|\|\|+\| +\| +\| +\|++\|\|\|+\|+\|\|\| ++\|+\|\|+\|+\| + | | |
| NOV21C | 70 | ENLLVICVNWR-GSGRaGLMNLYILNMAIADLGIVLSLPVWMLEVTL | 115 | |
| | | ggsedWpfGsalCklvtaldvvnmyaSil<-* | | (SEQ ID NO:309X) |
| | | ++\|++\|\|+ \|++++++++\|\|\|\|+\|\|+ | | |
| NOV21C | | D--YTWLWGSFSCRFTHYFYFVNMYSSIF | 142 | (SEQ ID NO:310) |

7tm_1: domain 2 of 2, from 143 to 236: score 86.7, E = 13e-26

| | | | | |
|---|---|---|---|---|
| | | *->FllPllvilvcYtrIlrtlr........kaaktllvvvvvFvlCWlP | | |
| | | \|\|\|\|+ +\|+\|++ +++++\|\|+++++++++ + +\|+++\| \|\|\| \|\|\|\| | | |
| NOV21C | 143 | FLLPFPLITVFNVLTACRLRqpgqpksrRHCLLLCAYVAVFVMCWLP | 189 | |
| | | yfivllldtlc.lsiimsstCelervlptallvtlwLayvNsclNPilY< | | (SEQ ID NO:311) |
| | | \|+++\|\|\| \|\|++++\| \|++\| \| ++\| ++++\|+ +++++++++\|\|\|+\| | | |
| NOV21C | | YHVTLLLLTLHgTHI--SLHCHLVHLLYFFYDVIDCFSMLHCVINPILY | 236 | (SEQ ID NO:312) |

TABLE 21L
Domain Analysis of NOV21a

gnl|pfam|pfam00001, 7tm_1, 7 transmembrane receptor (rhodopsin family)
CD-Length = 254 residues, 100.0% aligned
Score = 147 bits (371), Expect = 1e−36

| | | | | |
|---|---|---|---|---|
| NOV21: | 70 | ENLLVICVNWRGSGRAGLMNLYILNMAIADLGIVLSLPVWMLEVTLDYTWLWGSFSCRFT | 129 | (SEQ ID NO:313) |
| | | \|\|\|\|\| \| \| \|+++\|\|+\|++\|\|\| +\|+\|\| \| \| + \|++\| \|+ | | |
| Sbjct: | 1 | GNLLVILVILRTKKLRTPTNIFLLNLAVADLLFLLTLPPWALYYLVGGDWVFGDALCKLV | 60 | (SEQ ID NO:314) |
| NOV21: | 130 | HYFYFVNMYSSIFFLVCLSVDRYVTLTSASPSWORYQHRVRRAMCAGIWVLSAIIPLPEV | 189 | |
| | | + \|\| \|+\|\| \| +\|+\|\|\|+ + + \| + + +\|\|\|+ ++ \|\| + | | |
| Sbjct: | 61 | GALFVVNGYASILLLTAISIDRYIAIVHPLRYRRIRTPRRAKVLILLVWVLALLLSLPPL | 120 | |
| NOV21: | 190 | VHIQLVEGPEPMCLFMAPFETYSTWALAVALSTTILGFLLPFPLITVFNVLTACRLRQPG | 249 | |
| | | + \| \| + + \| +\|++\|\|+\|\| +\| \| \|\|+ | | |
| Sbjct: | 121 | LFSWLRTVEEGNTTVCLIDFPEESVKRSYVLLSTLVGFVLPLLVILVCYTRILRTLRKEA | 180 | |
| NOV21: | 250 | QP---------KSRRHCLLLCAYVAVFVMCWLPYHVTLLLLTLHGTHISLHCHLVHLLYF | 300 | |
| | | + \|+ +\| \| \|\|\|+\|\| \| + + +\| | | |
| Sbjct: | 181 | RSQRSLKRRSSSERKAAKMLLVVVVVFVLCW------LPYHIVLLLDSLCLLSIWRVLPT | 234 | |
| NOV21: | 301 | FYDVIDCFSMLHCVINPILY | 320 | |
| | | + + ++ +\|\|\|+\| | | |
| Sbjct: | 235 | ALLITLWLAYVNSCLNPIIY | 254 | |

The rhodopsin-like GPCRs themselves represent a widespread protein family that includes hormone, neurotransmitter and light receptors, all of which transduce extracellular signals through interaction with guanine nucleotide-binding (G) proteins. Although their activating ligands vary widely in structure and character, the amino acid sequences of the receptors are very similar and are believed to adopt a common structural framework comprising 7 transmembrane (TM) helices. See InterPro IPR000276.

G-protein-coupled receptors (GPCRs) constitute a vast protein family that encompasses a wide range of functions (including various autocrine, paracrine and endocrine processes). They show considerable diversity at the sequence level, on the basis of which they can be separated into distinct groups. The term clan is used to describe the GPCRs, as they embrace a group of families for which there are indications of evolutionary relationship, but between which there is no statistically significant similarity in sequence. The currently known clan members include the rhodopsin-like GPCRs, the secretin-like GPCRs, the cAMP receptors, the fungal mating pheromone receptors, and the metabotropic glutamate receptor family.

Adrenomedullin (AM, or ADM; 103275) is a 52-amino acid peptide involved in vasodilation and body fluid homeostasis. By PCR on human genomic DNA using primers based on the rat ADM receptor (Admr), Hanze et al. (1997) isolated a cDNA encoding human ADMR, which they called AMR. Sequence analysis predicted that the 404-amino acid, 7-transmembrane ADMR protein, which is 73% identical to the rat ADM receptor, contains 2 potential N-terminal N-linked glycosylation sites and several potential ser and thr C-terminal cytoplasmic phosphorylation sites. Northern blot analysis detected highest expression of a major 1.8-kb ADMR transcript in heart, skeletal muscle, liver, pancreas, stomach, spleen, lymph node, bone marrow, adrenal gland, and thyroid, with lower expression in brain, lung, placenta, small intestine, thymus, and leukocytes. Southern blot analysis indicated that ADMR is a single-copy gene. See Hanze, et al., Biochem. Biophys. Res. Commun. 240:183-188, 1997, PubMed ID: 9367907.

The protein similarity information, expression pattern, cellular localization, and map location for the NOV21 protein and nucleic acid disclosed herein suggest that this Adrenomedullin Receptor-like protein may have important structural and/or physiological functions characteristic of the Adrenomedullin Receptor family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV21 nucleic acids and proteins of the invention have applications in the diagnosis and/or treatment of various diseases and disorders. For example, the compositions of the present invention will have efficacy for the treatment of patients suffering from: developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders; control of feeding; potential obesity due to over-eating; potential disorders due to starvation (lack of appetite), non-insulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome and/or other pathologies and disorders of the like. The polypeptides can be used as immunogens to produce antibodies specific for the invention, and as vaccines. They can also be used to screen for potential agonist and antagonist compounds. For example, a cDNA encoding the adrenomedullin-like protein may be useful in gene therapy, and the adrenomedullin-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding adrenomedullin-like protein, and the adrenomedullin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation; Colon cancer, Colorectal cancer; Colorectal cancer; familial nonpolyposis, type 6; Esophageal cancer; Hepatoblastoma; Hypobetalipoproteinemia, familial, 2; Lung cancer; Metaphyseal chondrodysplasia, Murk Jansen type; Ovarian carcinoma, endometrioid type; Pilomatricoma; Pseudo-Zellweger syndrome as well as other diseases, disorders and conditions.

These antibodies may be generated according to methods known in the art, using prediction from hydrophobicity charts, as described in the “Anti-NOVX Antibodies” section below. The disclosed NOV21 protein has multiple hydrophilic regions, each of which can be used as an immunogen. In one embodiment, a contemplated NOV21 epitope is from about amino acids 10 to 40. In another embodiment, a contemplated NOV21 epitope is from about amino acids 160 to 165. In other specific embodiments, contemplated NOV21 epitopes are from about amino acids 250 to 265, 270 to 280 and 300 to 320.

NOV22

One NOVX protein of the invention, referred to herein as NOV22, includes two Tyrosine Phosphatase-like proteins. The disclosed proteins have been named NOV22a, and NOV22b.

NOV22a

A disclosed NOV22a (designated CuraGen Acc. No. CG57256-01), which encodes a novel Protein Tyrosine Phosphatase-like protein and includes the 549 nucleotide sequence (SEQ ID NO:69) is shown in Table 22A. An open reading frame for the mature protein was identified beginning with an ATG initiation codon at nucleotides 30-32 and ending with a TAA stop codon at nucleotides 540-542. Putative untranslated regions are underlined in Table 22A, and the start and stop codons are in bold letters.

TABLE 22A
NOV22a Nucleotide Sequence

(SEQ ID NO:69)

TATTTTTTAACTAAATTAATACACCTCGAATGAACCACCCAGCTCCTGTGAAAGTCACATACAAGAACATGAGA
TTTCCTATTACACACAATCCAACCAATGTGACCTTAAATAAATTTATAGAGGAGCTTAAGAAGTATGGAGCTAC
CACAATAGTAAGAGTATGTGAAGCAACTTATGACACTACTCTTGTGGAGAAAGAAGGTATCCATGTTCTCAATT
GGCCTTTTGGTGATGGTGCACCACCATCCAACCAGATTGTTGCTGATTGGTTACATTTTGTAAAAATTAAGTTT
TGTGAAGAACCTGGTTGTTATATTGCTGTTAATTGCATTGTAGGCCTTGGGAAAGCTCCAGTACTTGTTGCCCT
AGCATCAGTTGAAGGTGGAATGAAACATGAAGATGCAGTACAATTCATAGGACAAAAGCGGAGTGGAGCTTTTA
AAAGCAAGCAACTTTTGTATTTGGAGAAGTATCATCCTAAAATGCGGCTGCGCTTCAAAGATTCCAATAGTCAT
ATAAACAACTGTTGCATTCAATAAAACTGGG

The disclosed NOV22a nucleic acid sequence maps to chromosome 1 and has 505 of 546 bases (92%) identical to a gb:GENBANK-ID:HSU48296|acc:U48296.1 mRNA from Homo sapiens (Homo sapiens protein tyrosine phosphatase PTPCAAX1 (hPTPCAAX1) mRNA, complete cds) (E=9.8e⁻¹⁰¹).

A disclosed NOV22a polypeptide (SEQ ID NO:70) is 170 amino acid residues in length and is presented using the one-letter amino acid code in Table 22B. The SignalP, Psort and/or Hydropathy results predict that NOV22a does not have a signal peptide and is likely to be localized to the endoplasmic reticulum (membrane) with a certainty of 0.8500. In alternative embodiments, a NOV22a polypeptide is located to the plasma membrane with a certainty of 0.4400, the mitochondrial inner membrane with a certainty of 0.1000, or the Golgi body with a certainty of 0.1000.

TABLE 22B
Encoded NOV22a Protein Sequence

(SEQ ID NO:70)

MNHPAPVKVTYKNMRFPITHNPTNVTLNKFIEELKKYGATTIVRVCEATYDTTLVEKEGIHVLNWPFGDGAPPSNQ
IVADWLHFVKIKFCEEPGCYIAVNCIVGLGKAPVLVALASVEGGMKHEDAVQFIGQKRSGAFKSKQLLYLEKYHPK
MRLRFKDSNSHINNCCIQ

The NOV22a amino acid sequence was found to have 145 of 170 amino acid residues (85%) identical to, and 152 of 170 amino acid residues (89%) similar to, the 173 amino acid residue ptnr:SPTREMBL-ACC:000648 protein from Homo sapiens (Human) (PROTEIN TYROSINE PHOSPHATASE PTPCAAX1) (E=1.9e⁻⁷⁶).

NOV22a is predicted to be expressed in the liver because of the expression pattern of (GENBANK-ID: gb:GENBANK-ID:HSU48296|acc:48296.1), a closely related Homo sapiens protein tyrosine phosphatase PTPCAAX1 (hPTPCAAX1) mRNA, complete cds homolog in species Homo sapiens.

NOV22b

A disclosed NOV22b (designated CuraGen Acc. No. CG57256-02), which encodes a novel Protein Tyrosine Phosphatase-like protein and includes the 850 nucleotide sequence (SEQ ID NO:71) is shown in Table 22C. An open reading frame for the mature protein was identified beginning with an ATG initiation codon at nucleotides 1-3 and ending with a TAG stop codon at nucleotides 529-531. Putative untranslated regions are underlined in Table 22C, and the start and stop codons are in bold letters.

TABLE 22C
NOV22b Nucleotide Sequence

(SEQ ID NO:71)

ATGAACCACCCAGCTCCTGTGATGAACCACCCAGCTCCTGTGAAAGTCACATACAAGAACATGAGATTTCCTATTAC
ACACAATCCAACCAATGTGACCTTAAATAAATTTATAGAGGAGCTTAAGAAGTATGGAGCTACCACAATAGTAAGAG
TATGTGAAGCAACTTATGACACTACTCTTGTGGAGAAAGAAGGTATCCATGTTCTCAATTGGCCTTTTGGTGATGGT
GCACCACCATCCAACCAGATTGTTGCTGATTGGTTACATTTTGTAAAAATTAAGTTTTGTGAAGAACCTGGTTGTTA
TATTGCTGTTAATTGCATTGTAGGCCTTGGGAAAGCTCCAGTACTTGTTGCCCTAGCATCAGTTGAAGGTGGAATGA
AACATGAAGATGCAGTACAATTCATAGGACAAAAGCGGAGTGGAGCTTTTAAAAGCAAGCAACTTTTGTATTTGGAG
AAGTATCATCCTAAAATGCGGCTGCGCTTCAAAGATTCCAATAGTGCTGCGCTTCAAAGATTCCAATAGTGCTGCGC
TTCAAAGATTCCAATAGTGCTGCGCTTCAAAGATTCCAATAGTGCTGCGCTTCAAAGATTCCAATAGTGCTGCGCTT
CAAAGATTCCAATAGTGCTGCGCTTCAAAGATTCCAATAGTGCTGCGCTTCAAAGATTCCAATAGTGCTGCGCTTCA
AAGATTCCAATAGTGCTGCGCTTCAAAGATTCCAATAGTGCTGCGCTTCAAAGATTCCAATAGTGCTGCGCTTCAAA
GATTCCAATAGTGCTGCGCTTCAAAGATTCCAATAGTGCTGCGCTTCAAAGATTCCAATAGTGCTGCGCTTCAAAGA
TTC

The disclosed NOV22b nucleic acid sequence maps to chromosome 6q12 and has 452 of 486 bases (93%) identical to a gb:GENBANK-ID:HSU48296|acc:U48296.1 mRNA from Homo sapiens (Homo sapiens protein tyrosine phosphatase PTPCAAX1 (hPTPCAAX1) mRNA, complete cds) (E=2.8e⁻⁹⁰).

A disclosed NOV22b polypeptide (SEQ ID NO:72) is 176 amino acid residues in length and is presented using the one-letter amino acid code in Table 22D. The SignalP, Psort and/or Hydropathy results predict that NOV22b does not have a signal peptide and is likely to be localized to the endoplasmic reticulum (membrane) with a certainty of 0.8500. In alternative embodiments, a NOV22b polypeptide is located to the plasma membrane with a certainty of 0.8500, the microbody (peroxisome) with a certainty of 0.4400, or the mitochondrial inner membrane with a certainty of 0.1000.

TABLE 22D
Encoded NOV22b Protein Sequence

(SEQ ID NO:72)

MNHPAPVMNHPAPVKVTYKNMRFPITHNPTNVTLNKFIEELKKYGATTIVRVCEATYDTTLVEDEGIHVLNWPFGDG
APPSNQIVADWLHFVKIKFCEEPGCYIAVNCIVGLGKAPVLVALASVEGGMKHEDAVQFIGQKRSGAFKSKQLLYLE
KYHPKMRLRFKDSNSAALQRFQ

The NOV22b amino acid sequence was found to have 138 of 161 amino acid residues (85%) identical to, and 145 of 161 amino acid residues (90%) similar to, the 173 amino acid residue ptnr:SPTREMBL-ACC:000648 protein from Homo sapiens (Human) (PROTEIN TYROSINE PHOSPHATASE PTPCAAX1)(E=8.2e⁻⁷²).

NOV22b is expressed in at least the brain. Expression information was derived from the tissue sources of the sequences that were included in the derivation of the sequence of NOV22b. The sequence is also predicted to be expressed in the liver because of the expression pattern of (GENBANK-ID: gb:GENBANK-ID:HSU48296|acc:U48296.1), a closely related Homo sapiens protein tyrosine phosphatase PTPCAAX1 (hPTPCAAX1) mRNA, complete cds homolog in species Homo sapiens.

Homologies to any of the above NOV22a proteins will be shared by the other NOV22 proteins insofar as they are homologous to each other as shown above. Any reference to NOV22 is assumed to refer to both of the NOV22 proteins in general, unless otherwise noted.

NOV22a and NOV22b are very closely homologous as is shown in the amino acid alignment in Table 22E.

[image]

NOV22a also has homology to the amino acid sequences shown in the BLASTP data listed in Table 22F.

TABLE 22F
BLAST results for NOV22a

| Gene Index/ | | Length | Identity | Positives | |
|---|---|---|---|---|---|
| Identifier | Protein/Organism | (aa) | (%) | (%) | Expect |
| gi\|4506283\|ref\| | protein tyrosine | 173 | 145/170 | 152/170 | 3e−83 |
| NP_003454.1\| | phosphatase type | | (85%) | (89%) | |
| (NM_003463) | IVA, member 1; | | | | |
| | Protein tyrosine | | | | |
| | phosphatase IVA1 | | | | |
| | [Homo sapiens] | | | | |
| gi\|17528929\|gb\| | protein tyrosine | 173 | 144/170 | 151/170 | 5e−82 |
| AAL38661.1\| | phosphatase 4a1 | | (84%) | (88%) | |
| (AY062269) | [Rattus | | | | |
| | norvegicus] | | | | |
| gi\|4506285\|ref\| | protein tyrosine | 167 | 126/170 | 144/170 | 2e−72 |
| NP_003470.1\| | phosphatase type | | (74%) | (84%) | |
| (NM_003479) | IVA, member 2, | | | | |
| | isoform 1; protein | | | | |
| | tyrosine | | | | |
| | phosphatase IVA; | | | | |
| | protein tyrosine | | | | |
| | phosphatase IVA2; | | | | |
| | phosphatase of | | | | |
| | regenerating liver | | | | |
| | 2 [Homo sapiens] | | | | |
| gi\|1246236\|gb\| | ptp-IV1b, PTP-IV1 | 167 | 125/170 | 144/170 | 4e−72 |
| AB39331.1\| | gene product [Homo | | (73%) | (84%) | |
| (L48937) | sapiens] | | | | |
| gi\|7513774\|pir\| | prenylated protein | 167 | 124/170 | 143/170 | 2e−71 |
| \|JC5981 | tyrosine | | (72%) | (83%) | |
| | phosphatase (EC | | | | |
| | 3.1.3.-) 2 - mouse | | | | |

The homology of these sequences is shown graphically in the ClustalW analysis shown in Table 22G.

[image]

[image]

Table 22H lists the domain description from DOMAIN analysis results against NOV22. This indicates that the NOV22 sequence has properties similar to those of other proteins known to contain the protein tyrosine phosphatase domain and the protein tyrosine phosphatase catalytic domain motif.

TABLE 22H
Domain Analysis of NOV22

gnl|Pfam|pfam00102, Y_phosphatase, Protein-tyrosine phosphatase.
CD-Length = 235 residues,
Score = 44.3 bits (103), Expect = 6e−06

| | | | | |
|---|---|---|---|---|
| NOV22: | 17 | PITHNPTNVTLNKFIEELKKYGATTIVRVCEATYDTTLVEKEG--IHVLNWPFGDGAPPS | 74 | (SEQ ID NO:320) |
| | | +\|+ \|\| ++ \| \|\| \| \| + \| \|\| \| \| \| | | |
| Sbjct: | 96 | SLTYGDFTVTCVSVEKKKDDY----TVRTLELTNSGDDETRTVKHYHYTGWP-DHGVPES | 150 | (SEQ ID NO:321) |
| NOV22: | 75 | NQIVADWLHFVKIKFCEEPGCYIAVNCIVGLGKAPVLVALASV------EGGMKHEDAVQ | 128 | |
| | | + + \| \| \|+ \| \|+\| \|+\|+ +\|+ + \|\| + \| \|+ | | |
| Sbjct: | 151 | PKSILDLLRKVRKSKGTPDDGPIVVHCSAGIGRTGTFIAIDILLQQLEKEGVVDVFDTVK | 210 | |
| NOV22: | 129 | FIGQKRSGAFKS-KQLLYL | 146 | |
| | | + +\| \| ++ +\| +++ | | |
| Sbjct: | 211 | KLRSQRPGMVQTEEQYIFI | 229 | |

gnl|Smart|smart00404, PTPc_motif, Protein tyrosine phosphatase, catalytic
domain motif
CD-Length = 105 residues, 93.3% aligned
Score = 39.7 bits (91), Expect = 1e−04

| | | | | |
|---|---|---|---|---|
| NOV22: | 61 | HVLNWPFGDGAPPSNQIVADWLHFVKIKFCEEPGCY-IAVNCIVGLGKAPVLVALASV-- | 117 | (SEQ ID NO:322) |
| | | \| \|\| \| \| \| + ++\| \|\| + + \|+\| \|+\|+ \|\|+ + | | |
| Sbjct: | 6 | HYTGWPD-HGVPESPDSILEFLRAVKKSLNKSANNGPVVVHCSAGVGRTGTFVAIDILLQ | 64 | (SEQ ID NO:323) |
| NOV22: | 118 | -----EGGMKHEDAVQFIGQKRSGAFKSK-QLLYLEKYH | 150 | |
| | | \| + \| \|+ + +\| \|\| ++ \| \|+\| + | | |
| Sbjct: | 65 | QLEAGTGEVDIFDIVKELRSQRPGAVQTLEQYLFLYRAL | 103 | |

Cellular processes involving growth, differentiation, transformation and metabolism are often regulated in part by protein phosphorylation and dephosphorylation. The protein tyrosine phosphatases (PTPs), which hydrolyze the phosphate monoesters of tyrosine residues, all share a common active site motif and are classified into 3 groups. These include the receptor-like PTPs, the intracellular PTPs, and the dual-specificity PTPs, which can dephosphorylate at serine and threonine residues as well as at tyrosines. Diamond et al. (1994) described a PTP from regenerating rat liver that is a member of a fourth class. The gene, which they designated Prl1, was one of many immediate-early genes. Overexpression of Prl1 in stably transfected cells resulted in a transformed phenotype, which suggested that it may play some role in tumorigenesis. By using an in vitro prenylation screen, Cates et al. (1996) isolated 2 human cDNAs encoding PRL1 homologs, designated PTP(CAAX1) and PTP(CAAX2)(PRL2), that are farnesylated in vitro by mammalian farnesyl:protein transferase. Overexpression of these PTPs in epithelial cells caused a transformed phenotype in cultured cells and tumor growth in nude mice. The authors concluded that PTP(CAAX1) and PTP(CAAX2) represent a novel class of isoprenylated, oncogenic PTPs. Peng et al. (1998) reported that the human PTP(CAAX1) gene, or PRL1, is composed of 6 exons and contains 2 promoters. The predicted mouse, rat, and human PRL1 proteins are identical. Zeng et al. (1998)determined that the human PRL1 and PRL2 proteins share 87% amino acid sequence identity.

The protein similarity information, expression pattern, cellular localization, and map location for the protein and nucleic acid disclosed herein suggest that this Protein Tyrosine Phosphatase-like protein may have important structural and/or physiological functions characteristic of the Protein Tyrosin Phosphatase family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: Cardiomyopathy, dilated, 1K; cancer; on Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration; Von Hippel-Lindau (VHL) syndrome, cirrhosis, transplantation as well as other diseases, disorders and conditions. These materials are further useful in the generation of antibodies that bind immunospecifically to the novel substances of the invention for use in therapeutic or diagnostic methods.

These antibodies may be generated according to methods known in the art, using prediction from hydrophobicity charts, as described in the “Anti-NOVX Antibodies” section below. The disclosed NOV22 protein has multiple hydrophilic regions, each of which can be used as an immunogen. In one embodiment, a contemplated NOV22 epitope is from about amino acids 10-22. In another embodiment, a contemplated NOV22 epitope is from about amino acids 25-32. In other specific embodiments, contemplated NOV22 epitopes are from about amino acids 38 to 39, 40 to 43, 50 to 52, 53 to 55, 57 to 60, 65 to 70, 75 to 80, 82 to 83, 125 to 127, 128 to 132, 140 to 145 and 150 to 160.

NOV23

A disclosed NOV23 (designated CuraGen Acc. No. CG57228-01), which encodes a novel Aldo-Keto Reductase Family 7, member A3-like protein and includes the 1144 nucleotide sequence (SEQ ID NO:73) is shown in Table 23A. An open reading frame for the mature protein was identified beginning with an ATG initiation codon at nucleotides 55-57 and ending with a TAA stop codon at nucleotides 1120-1122. Putative untranslated regions are underlined in Table 23A, and the start and stop codons are in bold letters.

TABLE 23A
NOV23 Nucleotide Sequence

(SEQ ID NO:73)

TTCCGACCGCTGCGCGCGGCTCCTGGGCTGTCACAGTCTCCCGTTGCCGCCGTCATGTCCCGGCAGCTGTCGCGG
GCCCGGCCAGCCACGGTGCTGGGCGCCATGGAGATGGGGCGCCGCATGGACGCGCCCACCAGCGCCGCAGTCACG
CGCGCCTTCCTGGAGCGCGGCCACACCGAGATAGACACGGCCTTCCTGTACAGCGACGGCCAGTCCGAGACCATC
CTTGGCGGCCTGGGGCTCCGAATGGGCAGCAGCGACTGCAGAGTGAAAATTGCTACCAAGGCCAATCCATGGATT
GGGAACTCCCTGAAGCCTGACAGTGTCCGATCCCAGCTGGAGACGTCACTGAAGCGGCTGCAGTGTCCCAGAGTG
GACCTCTTCTATCTACATGCACCTGACCACAGCGCCCCGGTGGAAGAGACACTGCGTGCCTGCCACCAGCTGCAC
CAGGAGGGCAAGTTCGTGGAGCTTGGCCTCTCCAACTATGCCGCCTGGGAAGTGGCCGAGATCTGTACCCTCTGC
AAGAGCAACGGCTGGATCCTGCCCACTGTGTACCAGGGCATGTACAGCGCCACCACCCGGCAGGTGGAAACGGAG
CTCTTCCCCTGCCTCAGGCACTTTGGACTGAGGTTCTATGCCTACAACCCTCTGGCTGACCAGAGCCCTGAGGGA
TGTGGCAGCTTCTGGGGCACTCTGGGCCCGGGGGCTGATTGCTGCCTTCCCGCAGGGGGCCTGCTGACCGGCAAG
TACAAGTATGAGGACAAGGACGGGAAACAGCCCGTGGGCCGCTTCTTTGGGACTCAGTGGGCAGAGATCTACAGG
AATCAGTTCTGGAAGGAGCACCACTTCGAGGGCATTGCCCTGGTGGAGAAGGCCCTGCAGGCCGCGTATGGCGCC
AGCGCTCCCAGCATGACCTCGGCCGCCCTCCGGTGGATGTACCACCACTCACAGCTGCAGGGTGCCCACGGGGAC
GCGGTCATCCTGGGCATGTCCAGCCTGGAGCAGCTGGAGCAGAACTTGGCAGCGGCAGAGGAAGGGCCCCTGGAG
CCGGCTGTCGTGGACGCCTTTAATCAAGCCTGGCATTTGTTTGCCCACGAATGTCCCAACTACTTCATCTAAGCT
CATTGTGGCTCAGGCTGCC

The disclosed NOV23 nucleic acid sequence maps to chromosome 1 and has 632 of 658 bases (96%) identical to a gb:GENBANK-ID:AF040639|acc:AF040639.1 mRNA from Homo sapiens (Homo sapiens aflatoxin B 1-aldehyde reductase mRNA, complete cds) (E=5.2e⁻²¹⁶).

A disclosed NOV23 polypeptide (SEQ ID NO:74) is 355 amino acid residues in length and is presented using the one-letter amino acid code in Table 23B. The SignalP, Psort and/or Hydropathy results predict that NOV23 has a signal peptide and is likely to be localized to the microbody (peroxisome) with a certainty of 0.5268. In alternative embodiments, a NOV23 polypeptide is located to the mitochondrial matrix space with a certainty of 0.5048, the mitochondrial inner membrane with a certainty of 0.2262, or the mitochondrial intermembrane space with a certainty of 0.2262. The SignalP predicts a likely cleavage site for a NOV23 peptide between amino acid positions 8 and 9, i.e. at the sequence SRA-RP.

TABLE 23B
Encoded NOV23 Protein Sequence

(SEQ ID NO:74)

MSRQLSRARPARVLGAMEMGRRMDAPTSAAVTRAFLERGHTEIDTAFLYSDGQSETILGGLGLRMGSSDCRVKIAT
KANPWIGNSLKPDSVRSQLETSLKRLQCPRVKLFYLHAPDHSAPVEETLRACHQLHQEGKFVELGLSNYAAWEVAE
ICTLCKSNGWILPTVYQGMYSATTRQVETELFPCLRHFGLRFYAYNPLADQSPEGCGSFWGTLGPGADCCLPAGGL
LTGKYKYEDKDGKQPVGRFFGTQWAEIYRNQFWKEHHFEGIALVEKALQAAYGASAPSMTSAALRWMYHHSQLQGA
HGDAVILGMSSLEQLEQNLAAAEEGPLEPAVVDAFNQAWHLFAHECPNYFI

The NOV23 amino acid sequence was found to have 328 of 354 amino acid residues (92%) identical to, and 339 of 354 amino acid residues (95%) similar to, the 355 amino acid residue ptnr:SPTREMBL-ACC:Q9NUC3 protein from Homo sapiens (Human) (DJ657E11.3 (ALDO-KETO REDUCTASE FAMILY 7, MEMBER A3 (AFLATOXIN ALDEHYDE REDUCTASE))) (E=3.6e⁻¹⁸³).

NOV23 is predicted to be expressed in the following tissues because of the expression pattern of (GENBANK-ID: gb:GENBANK-ID:AF040639|acc:AF040639.1) a closely related Homo sapiens aflatoxin B1-aldehyde reductase mRNA, complete cds homolog in species Homo sapiens: pancreas, exocrine, adrenal gland, colon, ovary, uterus, prostate, stomach, eye, lymph, parathyroid, marrow, hepatocellular carcinoma.

NOV23 has homology to the amino acid sequences shown in the BLASTP data listed in Table 23C.

TABLE 23C
BLAST results for NOV23

| Gene Index/ | | Length | Identity | Positives | |
|---|---|---|---|---|---|
| Identifier | Protein/Organism | (aa) | (%) | (%) | Expect |
| gi\|6941683\|emb\| | dJ657E11.3 (aldo- | 355 | 328/354 | 339/354 | 0.0 |
| CAB72322.1\| | keto reductase | | (92%) | (95%) | |
| (AL035413) | family 7, member | | | | |
| | A3 (aflatoxin | | | | |
| | aldehyde | | | | |
| | reductase)) [Homo | | | | |
| | sapiens] | | | | |
| gi\|6912234\|ref\| | aldo-keto reductase | 331 | 308/354 | 317/354 | e−173 |
| NP_036199.1\| | family 7, member A3 | | (87%) | (89%) | |
| (NM_012067) | (aflatoxin aldehyde | | | | |
| | reductase) [Homo | | | | |
| | sapiens] | | | | |
| gi\|13627233\|ref\| | aldo-keto reductase | 331 | 306/354 | 316/354 | e−172 |
| XP_001439.2\| | family 7, member A3 | | (86%) | (88%) | |
| (XM_001439) | (aflatoxin aldehyde | | | | |
| | reductase) [Homo | | | | |
| | sapiens] | | | | |
| gi\|13627237\|ref\| | similar to | 330 | 292/346 | 302/346 | e−160 |
| XP_001438.2\| | AFLATOXIN B1 | | (84%) | (86%) | |
| (XM_001438) | ALDEHYDE REDUCTASE | | | | |
| | 1 (AFB1-AR 1) | | | | |
| | (ALDOKETOREDTUCTASE | | | | |
| | 7) (H. sapiens) | | | | |
| | [Homo sapiens] | | | | |
| gi\|4502021\|ref\| | aldo-keto reductase | 330 | 291/346 | 301/346 | e−159 |
| NP_003680.1\| | family 7, member A2 | | (84%) | (86%) | |
| (NM_003689) | (aflatoxin aldehyde | | | | |
| | reductase); | | | | |
| | aflatoxin betal | | | | |
| | aldehyde reductase | | | | |
| | [Homo sapiens] | | | | |

The homology of these sequences is shown graphically in the ClustalW analysis shown in Table 23D.

[image]

[image]

Table 23E lists the domain description from DOMAIN analysis results against NOV23. This indicates that the NOV23 sequence has properties similar to those of other proteins known to contain these domains.

TABLE 23E
Domain Analysis of NOV23

gnl|Pfam|pfam00248, aldo_ket_red, Aldo/keto reductase family. This family
includes a number of K+ ion channel beta chain regulatory domains - these
are reported to have oxidoreductase activity.
CD-Length = 282 residues, 86.9% aligned
Score = 143 bits (360), Expect = 2e−35

| | | | | |
|---|---|---|---|---|
| NOV23: | 10 | PATVLGAMEMGRRMDAPTSAAVTRAFLERGHTEIDTAFLYSDGQSETILGGL---GLRMG | 66 | (SEQ ID NO:329) |
| | | \| \|\| + \|+\| + +\| \|+ \|+ \|\|\| +\| +\| +\| \| \| | | |
| Sbjct: | 8 | PLLGLGTWKTPGRVDDEEAFEAVKAALDAGYRHFDTAEIY---GNEEEVGEAIKEALFEG | 64 | (SEQ ID NO:330) |
| NOV23: | 67 | SSDCRVKIATKANPWIGNSLKPDSVRSQLETSLKRLQCPRVDLFYLHAPDHS-----APV | 121 | |
| | | \| \| \| + \| \| \|\| \|\| \|\|\|\|\| \|\|\|+ +\| \|\| \|+ | | |
| Sbjct: | 65 | SGVVREDIFITSKLW-NTFHSPKHVREALEKSLKRLGLDYVDLYLIHWPDPLKPGDDVPI | 123 | |
| NOV23: | 122 | EETLRACHQLHQEGKFVELGLSNYAAWEVAEICTLCKSNGWILPTVYQGMYSATTRQVET | 181 | |
| | | \|\|\| +\| +\| \|\|\| +\|+\|\|++\| ++ \| + \| \| \| \| \| \|\| | | |
| Sbjct: | 124 | EETWKALEKLVDEGKVRSIGVSNFSAEQLEEALSEAGK---EPPVVNQVEYHPYLRQ--D | 178 | |
| NOV23: | 182 | ELFPCLRHFGLRFYAYNPLADQSPEGCGSFWGTLGPGADCCLPAGGLLTGKYKYEDKDGK | 241 | |
| | | \|\| + \|+ \|\|+\|\| \|\|\| | | |
| Sbjct: | 179 | ELRKFCKKHGIGVTAYSPL------------------------GSGLL------------ | 202 | |
| NOV23: | 242 | QPVGRFFGTQWAEIYRNQFWKEHHFEGIALVEKALQAAYGASAPSMTSAALRWMYHHSQL | 301 | |
| | | ++\|\| \| + \| + \|\|+ + \|\|\|\|+ | | |
| Sbjct: | 203 | ----------------DKFWSELGSPEL-LEDPALKKIAEKYGKTPAQVALRWVLQ---- | 241 | |
| NOV23: | 302 | QGAHGDAVILGMSS | 315 | |
| | | \| +\|\| \|+ | | |
| Sbjct: | 242 | ---RGVSVIPKSST | 252 | |

The masking of charged amino or carboxy groups by N-phthalidylation and O-phthalidylation has been used to improve the absorption of many drugs, including ampicillin and 5-fluorouracil. Following absorption of such prodrugs, the phthalidyl group is hydrolyzed to release 2-carboxybenzaldehyde (2-CBA) and the pharmaceutically active compound; in humans, 2-CBA is further metabolized to 2-hydroxymethylbenzoic acid by reduction of the aldehyde group. The enzyme responsible for the reduction of 2-CBA in humans is identified as human aldo-keto reductase (AKR), a homologue of rat aflatoxin B1-aldehyde reductase (rAFAR). Ireland et al. cloned human aldo-keto reductase (AKR) from a liver cDNA library, and together with the rat protein, establishes the AKR7 family of the AKR superfamily. Unlike its rat homologue, human AFAR (hAFAR) appears to be constitutively expressed in human liver, and is widely expressed in extrahepatic tissues. The deduced human and rat protein sequences share 78% identity and 87% similarity. Although the two AKR7 proteins are predicted to possess distinct secondary structural features which distinguish them from the prototypic AKR1 family of AKRs, the catalytic- and NADPH-binding residues appear to be conserved in both families. Certain of the predicted structural features of the AKR7 family members are shared with the AKR6 beta-subunits of voltage-gated K⁺-channels. In addition to reducing the dialdehydic form of aflatoxin B1-8,9-dihydrodiol, hAFAR shows high affinity for the gamma-aminobutyric acid metabolite succinic semialdehyde (SSA) which is structurally related to 2-CBA, suggesting that hAFAR could function as both a SSA reductase and a 2-CBA reductase in vivo. This hypothesis is supported in part by the finding that the major peak of 2-CBA reductase activity in human liver co-purifies with HAFAR protein.

Alterations of the distal portion of the short arm of chromosome 1 (1p) are among the earliest abnormalities of human colorectal tumors. Loss of heterozygosity analysis has previously revealed a smallest region of overlapping deletion (SRO) B, at 1p35-36.1, deleted in 48% of sporadic tumors. From this region Nishi et al. have cloned a gene encoding a protein of 330 amino acids that is 78% identical with the Rattus norvegicus aflatoxin B1 aldehyde reductase (Afar) and, therefore, likely represents its human homologue. In rat liver, Afar is strongly inducible by the antioxidants ethoxyquin and butylated hydroxyanisole, which protect the rat against aflatoxin B1-induced liver tumorigenesis by detoxifying its genotoxic and cytotoxic dialdehyde. Human AFAR is expressed in a broad range of tissues and, therefore, is likely involved in endogenous detoxication pathways. Impaired detoxication of genotoxic aldehydes and ketones, which are involved in tumorigenesis of the colon and breast, may be a crucial factor both for tumor initiation and progression.

The novel human Aldo-Keto Reductase Family 7, member A3-like Proteins of the invention contains aldo/keto reductase family domain and share 96% homology to human Aldo-Keto Reductase Family 7, member A3. Therefore it is anticipated that this novel protein has a role in the regulation of essentially all cellular functions and could be a potentially important target for drugs. Such drugs may have important therapeutic applications, such as treating numerous tumors. See, generally, Kelly et al., Endocrinology September 2000;141(9):3194-9; and Praml et al., Cancer Res Nov. 15, 1998;58(22):5014-8.

The protein similarity information, expression pattern, cellular localization, and map location for the NOV23 protein and nucleic acid disclosed herein suggest that this Aldo-Keto Reductase Family 7, member A3 like protein-like protein may have important structural and/or physiological functions characteristic of the Aldo-Keto Reductase Family 7 family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV23 nucleic acids and proteins of the invention have applications in the diagnosis and/or treatment of various diseases and disorders. For example, the compositions of the present invention will have efficacy for the treatment of patients suffering from: hemophilia, hypercoagulation, idiopathic thrombocytopenic purpura, autoimmune disease, allergies, immunodeficiencies, transplantation, graft versus host disease, allergies, lymphaedema, hypercalceimia, ulcers, fertility, endometriosis, diabetes, Von Hippel-Lindau (VHL) syndrome, pancreatitis, obesity, hypoparathyroidism, adrenoleukodystrophy, congenital adrenal hyperplasia, diabetes, tuberous sclerosis as well as other diseases, disorders and conditions.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV23 protein has multiple hydrophilic regions, each of which can be used as an immunogen. In one embodiment, a contemplated NOV23 epitope is from about amino acids 5 to 10. In another embodiment, a contemplated NOV23 epitope is from about amino acids 20 to 35. In other specific embodiments, contemplated NOV23 epitopes are from about amino acids 40 to 48, 60 to 62, 75 to 100, 110 to 140, 170 to 190, 195 to 215, 235 to 260, 292 to 305, 320 to 325, 340 to 342 and 348 to 349.

NOV24

A disclosed NOV24 (designated CuraGen Acc. No. CG57274-01), which encodes a novel Ral Guanine Nucleotide Exchange Factor 3-like protein and includes the 2171 nucleotide sequence (SEQ ID NO:75) is shown in Table 24A. An open reading frame for the mature protein was identified beginning with an ATG initiation codon at nucleotides 26-28 and ending with a TGA stop codon at nucleotides 2150-2152. Putative untranslated regions are underlined in Table 24A, and the start and stop codons are in bold letters.

TABLE 24A
NOV24 Nucleotide Sequence

(SEQ ID NO:75)

CCACTGAGAGGGACGGGCGCCGGCCATGGAGCGCACAGCAGGCAAAGAGCTGGCCGCACCGCTGCAGGACTGGGGT
GAAGAGACCGAGGACGGCGCGGTGTACAGTGTCTCCCTGCGGCGGCAGCGCAGTCAGCGCTCAGATCACCAGAGGT
CAGGAGTTGGACAGGCTCCCAGCCCCATTGCCAATACCTTCCTCCACTATCGAACCAGCAAGGTGAGGGTGCTGAG
GGCAGCGCGCCTGGAGCGGCTGGTGGGAGAGTTGGTGTTTGGAGACCGTGAGCAGGACCCCAGCTTCATGCCCGCC
TTCCTGGCCACCTACCGGACCTTTGTACCCACTGCCTGCCTGCTGGGCTTTCTGCTGCCACCAATGCCACCGCCCC
CACCTCCCGGGGTAGAGATCAAGAAGACAGCGGTACAAGATCTGAGCTTCAACAAGAACCTGAGGGCTGTGGTGTC
AGTGCTGGGCTCCTGGCTGCAGGACCACCCTCAGGATTTCCGAGACCCCCCTGCCCATTCGGACCTGGGCAGTGTC
CGAACCTTTCTGGGCTGGGCGGCCCCAGGGAGTGCTGAGGCTCAAAAAGCAGAGAAGCTTCTGGAAGATTTTTTGG
AGGAGGCTGAGCGAGAGCAGGAAGAGGAGCCGCCTCAGGTGTGGTCAGGACCTCCCAGAGTTGCCCAAACTTCTGA
CCCAGACTCTTCAGAGGCCTGCGCGGAGGAAGAGGAAGGGCTCATGCCTCAAGGTCCCCAGCTCCTGGACTTCAGC
GTGGACGAGGTGGCCGAGCAGCTGACCCTCATAGACTTGGAGCTCTTCTCCAAGGTGAGGCTCTACGAGTGCTTGG
GCTCCGTGTGGTCGCAGAGGGACCGGCCGGGGGCTGCAGGCGCCTCCCCCACTGTGCGCGCCACCGTGGCCCAGTT
CAACACCGTGACCGGCTGTGTGCTGGGTTCCGTGCTCGGAGCACCGGGCTTGGCCGCCCCGCAGAGGGCGCAGCGG
CTGGAGAAGTGGATCCGCATCGCCCAGCGCTGCCGAGAACTGCGGAACTTCTCCTCCTTGCGCGCCATCCTGTCCG
CCCTGCAATCTAACCCCATCTACCGGCTCAAGCGCAGCTGGGGGGCAGTGAGCCGGGAACCGCTATCTACTTTCAG
GAAACTTTCGCAGATTTTCTCCGATGAGAACAACCACCTCAGCAGCAGAGAGATTCTTTTCCAGGAGGAGGCCACT
GAGGGATCCCAAGAAGAGGACAACACCCCAGGCAGCCTGCCCTCAAAACCACCCCCAGGCCCTGTCCCCTACCTTG
GCACCTTCCTTACGGACCTGGTTATGCTGGACACAGCCCTGCCGGATATGTTGGAGGGGGATCTCATTAACTTTGA
GAAGAGGAGGAAGGAGTGGGAGATCCTGGCCCGCATCCAGCAGCTGCAGAGGCGCTGTCAGAGCTACACCCTGAGC
CCCCACCCGCCCATCCTGGCTGCCCTGCATGCCCAGAACCAGCTCACCGAGGAGCAGAGCTACCGGCTCTCCCGGG
TCATTGAGCCACCAGCTGCCTCCTGCCCCAGCTCCCCACGCATCCGACGGCGGATCAGCCTCACCAAGCGTCTCAG
TGCGAAGCTTGCCCGAGAGAAAAGCTCATCACCTAGTGGGAGTCCCGGGGACCCCTCATCCCCCACCTCCAGTGTG
TCCCCAGGGTCACCCCCCTCAAGTCCTAGAAGCAGAGATGCTCCTGCTGGCAGTCCCCCGGCCTCTCCAGGGCCCC
AGGGCCCCAGCACCAAGCTGCCCCTGAGCCTGGACCTGCCCAGCCCCCGGTCCCCCGTAACCCTAGACCCCTTTAG
CGCCCGGGTCCCTCTACCGGCGCAGCAGAGCTCGGAGGCCCGTGTCATCCGCGTCAGCATCGACAATGACCACGGG
AACCTGTATCGAAGCATCTTGCTGACCAGTCAGGACAAAGCCCCCAGCGTGGTCCGGCGAGCCTTGCAGAAGCACA
ATGTGCCCCAGCCCTGGGCCTGTGACTATCAGCTCTTTCAAGTCCTTCCTGGGGACCGGCTCCTGATTCCTGACAA
TGCCAACGTCTTCTATGCCATGAGTCCAGTCGCCCCCAGAGACTTCATGCTGCGGCGGAAAGAGGGGACCCGGAAC
ACTCTGTCTGTCTCCCCAAGCTGAGGCAGCCCTGTCCTCTCCA

The disclosed NOV24 nucleic acid sequence maps to chromosome 19 and has 1552 of 2159 bases (71%) identical to a gb:GENBANK-ID:AF237669|acc:AF237669.1 mRNA from Mus musculus (Mus musculus RaIGDS-like protein 3 mRNA, complete cds) (E=4.8e⁻¹⁸⁹).

A disclosed NOV24 polypeptide (SEQ ID NO:76) is 708 amino acid residues in length and is presented using the one-letter amino acid code in Table 24B. The SignalP, Psort and/or Hydropathy results predict that NOV24 does not have a signal peptide and is likely to be localized to the microbody (peroxisome) with a certainty of 0.3000. In alternative embodiments, a NOV24 polypeptide is located to the nucleus with a certainty of 0.3000, the mitochondrial matrix space with a certainty of 0.1000, or the lysosome (lumen) with a certainty of 0.1000.

TABLE 24B
Encoded NOV24 Protein Sequence

(SEQ ID NO:76)

MERTAGKELAAPLQDWGEETEDGAVYSVSLRRQRSQRSDHQRSGVGQAPSPIANTFLHYRTSKVRVLRAARLERL
VGELVFGDREQDPSFMPAFLATYRTFVPTACLLGFLLPPMPPPPPPGVEIKKTAVQDLSFNKNLRAVVSVLGSWL
QDHPQDFRDPPAHSDLGSVRTFLGWAAPGSAEAQKAEKLLEDFLEEAEREQEEEPPQVWSGPPRVAQTSDPDSSE
ACAEEEEGLMPQGPQLLDFSVDEVAEQLTLIDLELFSKVRLYECLGSVWSQRDRPGAAGASPTVRATVAQFNTVT
GCVLGSVLGAPGLAAPQRAQRLEKWIRIAQRCRELRNFSSLRAILSALQSNPIYRLKRSWGAVSREPLSTFRKLS
QIFSDENNHLSSREILFQEEATEGSQEEDNTPGSLPSKPPPGPVPYLGTFLTDLVMLDTALPDMLEGDLINFEKR
RKEWEILARIQQLQRRCQSYTLSPHPPILAALHAQNQLTEEQSYRLSRVIEPPAASCPSSPRIRRRISLTKRLSA
KLAREKSSSPSGSPGDPSSPTSSVSPGSPPSSPRSRDAPAGSPPASPGPQGPSTKLPLSLDLPSPRSPVTLDPFS
ARVPLPAQQSSEARVIRVSIDNDHGNLYRSILLTSQDKAPSVVRRALQKHNVPQPWACDYQLFQVLPGDRLLIPD
NANVFYAMSPVAPRDFMLRRKEGTRNTLSVSPS

The NOV24 amino acid sequence was found to have 577 of 709 amino acid residues (81%) identical to, and 629 of 709 amino acid residues (88%) similar to, the 709 amino acid residue ptnr:SPTREMBL-ACC:Q9JID4 protein from Mus musculus (Mouse) (RALGDS-LIKE PROTEIN 3) (E=5.9e⁻³⁰²).

NOV24 is expressed in at least the following tissues: Mammary gland/Breast, Uterus, Thyroid, Cartilage, Adrenal Gland/Suprarenal gland, Kidney, Liver, Lymph node, Pancreas, Substantia Nigra, Epidermiis, Cervix, Colon, Lung, Parathyroid Gland, and Whole Organism. Expression information was derived from the tissue sources of the sequences that were included in the derivation of the sequence of NOV24.

NOV24 has homology to the amino acid sequences shown in the BLASTP data listed in Table 24C.

TABLE 24C
BLAST results for NOV24

| Gene Index/ | | Length | Identity | Positives | |
|---|---|---|---|---|---|
| Identifier | Protein/Organism | (aa) | (%) | (%) | Expect |
| gi\|15186754\|gb\| | RalGDS-related | 709 | 577/714 | 629/714 | 0.0 |
| AAK91126.1\| | effector protein | | (80%) | (87%) | |
| AF239661_1 | of M-Ras [Mus | | | | |
| (AF239661) | musculus] | | | | |
| gi\|12963751\|ref\| | RalGDS-like | 709 | 576/714 | 628/714 | 0.0 |
| NP_076111.1\| | protein 3; Ral | | (80%) | (87%) | |
| (NM_023622) | guanine-nucleotide | | | | |
| | exchange factor | | | | |
| | [Mus musculus] | | | | |
| gi\|12836390\|dbj\| | RALGDS-LIKE | 343 | 251/320 | 279/320 | e−127 |
| BAB23634.1\| | PROTEIN 3˜data | | (78%) | (86%) | |
| (AK004876) | source: SPTR, | | | | |
| | source key: Q9JID4, | | | | |
| | evidence: ISS˜putative | | | | |
| | [Mus musculus] | | | | |
| gi\|14717390\|ref\| | RalGDS-like | 768 | 285/739 | 409/739 | e−120 |
| NP_055964.1\| | protein [Homo | | (38%) | (54%) | |
| (NM_015149) | sapiens] | | | | |
| gi\|10185686\|gb\| | RalGDS-like [Homo | 768 | 285/739 | 409/739 | e−120 |
| AAG14400.1\| | sapiens] | | (38%) | (54%) | |
| AF186798_1 | | | | | |
| (AF186798) | | | | | |

The homology of these sequences is shown graphically in the ClustalW analysis shown in Table 24D.

[image]

[image]

[image]

Table 24E lists the domain description from DOMAIN analysis results against NOV24. This indicates that the NOV24 sequence has properties similar to those of other proteins known to contain these Ras-related domains.

TABLE 24E
Domain Analysis of NOV24

gnl|Smart|smart00147, RasGEF, Guanine nucleotide exchange factor for Ras-
like small GTPases
CD-Length = 242 residues, 98.8% aligned
Score = 216 bits (551), Expect = 3e−57

| | | | | |
|---|---|---|---|---|
| NOV24: | 241 | LLDFSVDEVAEQLTLIDLELFSKVRLYECLGSVWSQRDRPGAAGASPTVRATVAQFNTVT | 300 | (SEQ ID NO:336) |
| | | \|\| \|+\|\|\|\|\|\|+\| \|\|\| \|+ \| \|\|\|\|\| +\| + + + + +\|\| \|+ | | |
| Sbjct: | 1 | LLLLDPKELAEQLTLLDFELFRKIDPSELLGSVWGKRSKKS--PSPLNLERFIERFNEVS | 58 | (SEQ ID NO:337) |
| NOV24: | 301 | GCVLGSVLGAPGLAAPQRAQRLEKWIRIAQRCRELRNFSSLRAILSALQSNPIYRLKRSW | 360 | |
| | | \| +\| \|\|+ \| \|+\|++\|+ \|\|\|\| \|\|+\|\| \|\|+\|\|\| \|+\|\| \|\|\|++\| | | |
| Sbjct: | 59 | NWVATEILKQTTP--KDRAELLSKFIQVAKHCRELNNFNSLMAIVSALSSSPISRLKKTW | 116 | |
| NOV24: | 361 | GAVSREPLSTFRKLSQIFSDENNHLSSREILFQEEATEGSQEEDNTPGSLPSKPPPGPVP | 420 | |
| | | + + \| +\| ++ \| + \|\| \| \| +\| | | |
| Sbjct: | 117 | EKLPSKYKKLFEELEELLDPSRNFKNYREALSSCN-------------------LPPCIP | 157 | |
| NOV24: | 421 | YLGTFLTDLVMLDTALPDMLEGDLINFEKRRKEWEILARIQQLQRRCQSYTLSPHPPILA | 480 | |
| | | +\|\| \| \|\| +\| \|\| \|+ \|+\|\|\|\|\|\|\| +\|\| \|+\|\|\| \| \| \| \|+ + | | |
| Sbjct: | 158 | FLGVLLKDLTFIDEGNPDFLKNGLVNFEKRRKIAKILREIRQLQS--QPYNLRPNRSDIQ | 215 | |
| NOV24: | 481 | AL--HAQNQLTEEQ-SYRLSRVIE 501 | | |
| | | +\| + + \| \|\| \| \|\| \|\| | | |
| Sbjct: | 216 | SLLQQSLDSLPEENELYELSLRIE 239 | | |

gnl|Pfam|pfam00617, RasGEF, RasGEF domain. Guanine nucleotide exchange
factor for Ras-like small GTPases.
CD-Length = 188 residues, 100.0% aligned
Score = 181 bits (459), Expect = 1e−46

| | | | | |
|---|---|---|---|---|
| NOV24: | 242 | LDFSVDEVAEQLTLIDLELFSKVRLYECLGSVWSQRDRPGAAGASPTVRATVAQFNTVTG | 301 | (SEQ ID NO:338) |
| | | \| \|+\|+\|\|\|\|++ \|\|\| \|+ +\|\|\|\| \|\| ++ \| \|\| + \|+ \|\| +\| | | |
| Sbjct: | 1 | LLLDPLELAKQLTLLEHELFKKIDPFECLGQVWGKKY--GKNERSPNIDKTIKNFNQLTN | 58 | (SEQ ID NO:339) |
| NOV24: | 302 | CVLGSVLGAPGLAAPQRAQRLEKWIRIAQRCRELRNFSSLRAILSALQSNPIYRLKRSWG | 361 | |
| | | \| ++\| +\|\|+ ++\|+\|++\| \|\|\|\| \|\|+\|\| \|\|+\|\|\| \|+\|\|\|\|\|\|++\| | | |
| Sbjct: | 59 | FVGTTILLQ--TDPKKRAELIQKFIQVADHCRELNNFNSLLAIISALYSSPIYRLKKTWQ | 116 | |
| NOV24: | 362 | AVSREPLSTFRKLSQIFSDENNHLSSREILFQEEATEGSQEEDNTPGSLPSKPPPGPVPY | 421 | |
| | | \| + \| \| +\|+++ + \| + \|\|+\| \| \| \|\|+ | | |
| Sbjct: | 117 | YVPPQSLKLFEELNKLMDSDRNFSNYRELL-------------------KSIFPLPCVPF | 157 | |
| NOV24: | 422 | LGTFLTDLVMLDTALPDMLEGDLINFEKRRK 452 | | |
| | | \| +\|+\|\| \|+ \|\| \|\| +\|+\|\| \|\|\|\| | | |
| Sbjct: | 158 | FGVYLSDLTFLEEGNPDFLETNLVNFSKRRK 188 | | |

gnl|Pfam|pfam00788, RA, Ras association (RalGDS/AF-6) domain. RasGTP
effectors (in cases of AF6, canoe and RalGDS); putative RasGTP effectors in
other cases. Recent evidence (not yet in MEDLINE) shows that some RA domains
do NOT bind RasGTP. Predicted structure similar to that determined, and that
of the RasGTP-binding domain of Raf kinase.
CD-Length = 92 residues, 96.7% aligned
Score = 62.4 bits (150), Expect = 8e−11

| | | | | |
|---|---|---|---|---|
| NOV24: | 615 | VIRVSIDNDH-GNLYRSILLTSQDKAPSVVRRALQKHNVPQPWACDYQLFQVLPGDRLL- | 672 | (SEQ ID NO:340) |
| | | \|+\|\| + \| \|++\| ++\|+\| \|\| \|\|+ \|\|+\| + +\| \| +\|\| \|+ | | |
| Sbjct: | 4 | VLRVYFQDLKPGVAYKTIRVSSEDTAPDVVQLALEKFRLDDEDPEEYALVEVLSGDKERK | 63 | (SEQ ID NO:341) |
| NOV24: | 673 | IPDNANVFYAM----SPVAPRDFMLRRKE 697 | | |
| | | +\|\|+ \| \|+\|+\|++ | | |
| Sbjct: | 64 | LPDDENPLQLRLNLPRDGLSLRFLLKRRD 92 | | |

gnl|Smart|smart00314, RA, Ras association (RaLGDS/AF-6) domain; RasGTP
effectors (in cases of AF6, canoe and RalGDS); putative RasGTP effectors in
other cases. Kalhammer et al. have shown that not all RA domains bind
RasGTP. Predicted structure similar to that determined, and that of the
RasGTP-binding domain of Raf kinase. Predicted RA domains in PLC210 and
norel found to bind RasGTP. Included outliers (Grb7, Grb14, adenylyl
cyclases etc.)
CD-Length = 90 residues, 95.6% aligned
Score = 56.2 bits (134), Expect = 6e−09

| | | | | |
|---|---|---|---|---|
| NOV24: | 615 | VIRVSIDNDHGNLYRSILLTSQDKAPSVVRRALQKHNVPQPWACDYQLFQVLPGDRLL-I | 673 | (SEQ ID NO:342) |
| | | \|+\|\| \| \| \| \|+++ ++ + \| \|+++ \|+\| ++ +\| \| +\| \| + + | | |
| Sbjct: | 4 | VLRVYFD-DPGGTYKTLRVSKRTTARDVIQQLLEKFHLTDDPE-EYVLVEVKEGGKERVL | 61 | (SEQ ID NO:343) |
| NOV24: | 674 | PDNANVFYAM----SPVAPRDFMLRRKE 697 | | |
| | | + + \|+\|\|+++ | | |
| Sbjct: | 62 | LPDEKPLQLQKLWPRQGSNLRFVLRKRD 89 | | |

gnl|Smart|smart00229, RasGEFN, Guanine nucleotide exchange factor for Ras-
like GTPases; N-terminal motif; A subset of guanine nucleotide exchange
factor for Ras-like small GTPases appear to possess this domain N-terminal
to the RasGef (Cdc25-like) domain. The recent crystal structure of Sos shows
that this domain is alpha-helical and plays a “purely structural role”
(Nature 394, 337-343).
CD-Length = 132 residues, 56.1% aligned
Score = 47.8 bits (112). Expect = 2e−06

| | | | | |
|---|---|---|---|---|
| NOV24: | 87 | DPSFMPAFLATYRTFVPTACLLGFLLPPMPPPPPPGVEIKKTAVQDLSFNKNLRAVVSVL | 146 | (SEQ ID NO:344) |
| | | \|\|+\|+ \|\| \|\|\|+\|+ \| \|\| \|\| \|\| \|\|\| + ++ + \|+++\| | | |
| Sbjct: | 26 | DPTFVETFLLTYRSFITTQELLQKLLYRYNAIPPEGVE-DIWVKEKVNPRRIQNRVLNIL | 84 | (SEQ ID NO:345) |
| NOV24: | 147 | GSWLQDHPQDFRDPP 161 | | |
| | | \|++++ \|\|\| + \| | | |
| Sbjct: | 85 | RLWVENYWQDFEEDP 99 | | |

RasGEF (See Interpro IPR001895; RasGEF domain) is a member of the Guanine-nucleotide dissociation stimulators CDC25 family. Ras proteins are membrane-associated molecular switches that bind GTP and GDP and slowly hydrolyze GTP to GDP. The balance between the GTP bound (active) and GDP bound (inactive) states is regulated by the opposite action of proteins activating the GTPase activity and that of proteins which promote the loss of bound GDP and the uptake of fresh GTP. The latter proteins are known as guanine-nucleotide dissociation stimulators (GDSs) (or also as guanine-nucleotide releasing (or exchange) factors (GRFs)). Proteins that act as GDS can be classified into at least two families, on the basis of sequence similarities, the CDC24 family (see INTERPRO IPR001331) and the CDC25 family.

The size of the proteins of the CDC25 family ranges from 309 residues (LTEI) to 1596 residues (sos). The sequence similarity shared by all these proteins is limited to a region of about 250 amino acids generally located in their C-terminal section (currently the only exceptions are sos and ralGDS where this domain makes up the central part of the protein). This domain has been shown, in CDC25 an SCD25, to be essential for the activity of these proteins.

Ras association (RalGDS/AF-6) domain, see RasGEFN (Interpro IPR000651; Guanine nucleotide exchange factor for Ras-1). The Guanine nucleotide exchange factor for Ras-like GTPases; N-terminal motif is found in several guanine nucleotide exchange factors for Ras-like small GTPases, and lies N-terminal to the RasGef (Cdc25-like) domain. Proteins belonging to this family include guanine nucleotide dissociation stimulator, which stimulates the dissociation of GDP from the Ras-related RaIA and RalB GTPases and allows GTP binding and activation of the GTPases; GTPase-activating protein (GAP) for Rhol and Rho2, which is involved in the control of cellular morphogenesis; and the yeast cell division control protein, which promotes the exchange of Ras-bound GDP by GTP and controls the level of cAMP when the cell division cycle is triggered. Also included is the son of sevenless protein, which promotes the exchange of Ras-bound GDP by GTP during neuronal development.

This indicates that the sequence of the invention has properties similar to those of other proteins known to contain these domains and similar to the properties of these domains.

The small GThase Rit is a close relative of Ras, and constitutively active Rit can induce oncogenic transformation. Although the effector loops of Rit and Ras are highly related, Rit fails to interact with the majority of the known Ras candidate effector proteins, suggesting that novel cellular targets may be responsible for Rit transforming activity. To gain insight into the cellular function of Rit, Shao and Andres (J Biol Chem 2000;275:26914-24) searched for Rit-binding proteins by yeast two-hybrid screening. They identified the C-terminal Rit/Ras interaction domain of a protein and designated as RGL3 (Ral GEF-like 3) that shares 35% sequence identity with the known Ral guanine nucleotide exchange factors (RaIGEFs). RGL3, through a C-terminal 99-amino acid domain, interacted in a GTP- and effector loop-dependent manner with Rit and Ras. Importantly, RGL3 exhibited guanine nucleotide exchange activity toward the small GTPase Ral that was stimulated in vivo by the expression of either activated Rit or Ras. These data suggest that RGL3 functions as an exchange factor for Ral and may serve as a downstream effector for both Rit and Ras (OMIM number: 601619).

Ras-related GTPases (see OMIM 190020) participate in signaling for a variety of cellular processes and are regulated in part by guanine nucleotide dissociation stimulators (GDSs, or exchange factors). Albright et al. (1993) used sequences derived from the yeast rasGDS proteins as probes and cloned cDNAs encoding a novel murine GDS protein. The protein stimulated the dissociation of guanine nucleotides from the ralA (179550) and ralB (179551) GTPases. The protein, designated RaIGDS by them, was at least 20-fold more active on the ralA and ralB GTPases than any other GTPases tested. The 3.6-kb ralGDS mRNA and the 115-kD ralGDS protein were found in all tissues examined.

Hofer et al. (1994) used a yeast 2-hybrid system to identify proteins in human that interact with Ras and isolated a gene encoding RALGDS, a protein which had previously been identified in mouse by Albright et al. (1993) as a guanine nucleotide exchange factor for the Ras-like molecule Ral. Hofer et al. (1994) reported that the interaction with Ras and Ras-like molecules was mediated by the C-terminal noncatalytic segment of RALGDS. They demonstrated that the interaction of the RALGDS C-terminal region with Ras is specific and dependent on the activation of Ras by GTP.

Independently, Spaargaren and Bischoff (1994) used a yeast 2-hybrid system to screen for proteins that bind to R-ras (165090). From this screen they obtained several clones that encoded the C-terminal region of the guanine nucleotide dissociation stimulator for Ral (RALGDS). Using the 2-hybrid system Spaargaren and Bischoff (1994) showed that the R-ras-binding domain of RALGDS interacts with H-ras, K-ras (190070), and Rap (RAP1A; 179520). Their data further indicated that RALGDS is a putative effector molecule for R-ras, H-ras, K-ras, and Rap.

Urano et al. (1996) demonstrated that ras-H(H-ras), R-ras, and Rap1A have the capacity to bind RalGDS in mammalian cells; however, only H-ras activates RalGDS. From these and other data they concluded that activation of RaIGDS and its target Ral constitutes a distinct downstream signaling pathway from H-ras that potentiates oncogenic transformation.

Schuler et al. (1996) generated a map of the human genome facilitated by the availability of expressed sequence tags (ESTs) mapping to radiation hybrid panels (see NCBI World Wide Web home page for more information). In their on-line map, they reported that ESTs (e.g., dbEST 785621; AA147088) representing a human homolog for the RALGDS gene map to chromosome 9q34 in the interval between D9S159 and D9S164 (see SCIENCE96 stSG2452).

The protein similarity information, expression pattern, cellular localization, and map location for the NOV24 protein and nucleic acid disclosed herein suggest that this Ral Guanine Nucleotide Exchange Factor 3-like protein may have important structural and/or physiological functions characteristic of the guanine nucleotide exchange factors family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV24 nucleic acids and proteins of the invention have applications in the diagnosis and/or treatment of various diseases and disorders. For example, the compositions of the present invention will have efficacy for the treatment of patients suffering from: cancer, trauma, tissue regeneration (in vitro and in vivo), viral/bacterial/parasitic infections, immunological disease, respiratory disease, gastro-intestinal diseases, reproductive health, neurological and neurodegenerative diseases, bone marrow transplantation, metabolic and endocrine diseases, allergy and inflammation, nephrological disorders, cardiovascular diseases, muscle, bone, joint and skeletal disorders, hematopoietic disorders, urinary system disorders as well as other diseases, disorders and conditions.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV24 protein has multiple hydrophilic regions, each of which can be used as an immunogen. In one embodiment, a contemplated NOV24 epitope is from about amino acids 2 to 40. In another embodiment, a contemplated NOV24 epitope is from about amino acids 65 to 90. In other specific embodiments, contemplated NOV24 epitopes are from about amino acids 115 to 120, 170 to 175, 195 to 230, 280 to 290, 310 to 320, 360 to 405, 460 to 475, 495 to 570, 605 to 660 and 690 to 695.

NOV25

A disclosed NOV25 (designated CuraGen Acc. No. CG57276-01), which encodes a novel Endolyn-like protein and includes the 717 nucleotide sequence (SEQ ID NO:77) is shown in Table 25A. An open reading frame for the mature protein was identified beginning with an ATG initiation codon at nucleotides 83-85 and ending with a TAA stop codon at nucleotides 668-670. Putative untranslated regions are underlined in Table 25A, and the start and stop codons are in bold letters.

TABLE 25A
NOV25 Nucleotide Sequence

(SEQ ID NO:77)

GAGGCGGCGCCGCAGGGGATTGAGGGGTTGACTGAGCGTTGCGAGCCTTAGCTTTCTCCCGAACGCCAGCGCTGAGG
ACACGATGTCGCGGCTCTCCCGCTCACTGCTTTGGGCCGCCACCTGCCTGGGCGTGCTCTGCGTGCTGTCCGCGGAC
AAGAACACGACCCAGCACCCGAACGTGACGACTTTAGCGCCCATCTCCAACGTAAAATCATTGATTTCATGCATCTC
TCCCCCCAACTCCCCAGAAACCTGTGAAGGTCGAAACAGCTGCGTTTCCTGTTTTAATGTTAGCGTTGTTAATACTA
CCTGCTTTTGGATAGAATGTCCCCCAACAGATGAGAGCTATTGTTCACATAACTCAACAGTTAGTGATTGTCAAGTG
GGGAACACGACAGACTTCTGTTCCGGTAAGTATTCATATTGGCTGCTTGGAAGCATTCCAGCTAAACCCACAGTTCA
GCCCTCCCCTTCTACAACTTCCAAGACAGTTACTACATCAGGTACAACAAATAACACTGTGACTCCAACCTCACAAC
CTGTGCGAAAGTCTACCTTTGATGCAGCCAGTTTCATTGGAGGAATTGTCCTGGTCTTGGGTGTGCAGGCTGTAATT
TTCTTTCTTTATAAATTCTGCAAATCTAAAGAACGAAATTACCACACTCTGTAAACAGACCCATTGAATTAATAAGG
ACTGGTGATTCATTTGTGTAACTC

The disclosed NOV25 nucleic acid sequence maps to chromosome 6 and has 495 of 649 bases (76%) identical to a gb:GENBANK-ID:RN02385741acc:AJ238574.1 mRNA from Rattus norvegicus (Rattus norvegicus mRNA for endolyn) (E=7.0e⁻⁶⁷).

A disclosed NOV25 polypeptide (SEQ ID NO:78) is 195 amino acid residues in length and is presented using the one-letter amino acid code in Table 25B. The SignalP, Psort and/or Hydropathy results predict that NOV25 has a signal peptide and is likely to be localized to the plasma membrane with a certainty of 0.4600. In alternative embodiments, a NOV25 polypeptide is located to the endoplasmic reticulum (membrane) with a certainty of 0.2800, the lysosome (membrane) with a certainty of 0.2000, or the endoplasmic reticulum (lumen) with a certainty of 0.1000. The SignalP predicts a likely cleavage site for a NOV25 peptide between amino acid positions 23 and 24, i.e. at the sequence LSA-DK.

TABLE 25B
Encoded NOV25 Protein Sequence

(SEQ ID NO:78)

MSRLSRSLLWAATCLGVLCVLSADKNTTQHPNVTTLAPISNVKSLISCISPPNSPETCEGRNSCVSCFNVSVVNTTC
FWIECPPTDESYCSHNSTVSDCQVGNTTDFCSGKYSYWLLGSIPAKPTVQPSPSTTSKTVTTSGTTNNTVTPTSQPV
RKSTFDAASFIGGIVLVLGVQAVIFFLYKFCKSKERNYHTL

The NOV25 amino acid sequence was found to have 110 of 195 amino acid residues (56%) identical to, and 136 of 195 amino acid residues (69%) similar to, the 195 amino acid residue ptnr:SPTREMBL-ACC:Q9QX82 protein from Rattus norvegicus (Rat) (ENDOLYN PRECURSOR) (E=7.2e⁻⁵²).

NOV25 is predicted to be expressed in the following tissues because of the expression pattern of (GENBANK-ID: gb:GENBANK-ID:RN0238574|acc:AJ238574.1), a closely related Rattus norvegicus mRNA for endolyn homolog in species Rattus norvegicus: testis, pancreas, lung, colon, kidney, skin, and breast.

Homologies to any of the above NOV25 proteins will be shared by other NOV25 proteins insofar as they are homologous to each other. Any reference to NOV25 is assumed to refer to NOV25 proteins in general, unless otherwise noted.

NOV25 also has homology to the amino acid sequences shown in the BLASTP data listed in Table 25C.

TABLE 25C
BLAST results for NOV25

| Gene Index/ | | Length | Identity | Positives | |
|---|---|---|---|---|---|
| Identifier | Protein/Organism | (aa) | (%) | (%) | Expect |
| gi\|12483942\|gb\| | CD164 isoform | 184 | 70/199 | 174/199 | 1e−63 |
| AAG53905.1\| | delta 4 [Homo | | (85%) | (87%) | |
| (AF299340) | sapiens] | | | | |
| gi\|9230741\|gb\| | CD164 [Homo | 197 | 170/199 | 174/199 | 9e−62 |
| AAF85965.1\|AF263279_1 | sapiens] | | (85%) | (87%) | |
| (AF263279) | | | | | |
| gi\|3941728\|gb\| | sialomucin CD164 | 178 | 154/198 | 158/198 | 1e−60 |
| AAC82473.1\| | [Homo sapiens] | | (77%) | (79%) | |
| (AF106518) | | | | | |
| gi\|5174407\|ref\| | CD164 antigen, | 189 | 147/179 | 153/179 | 3e−49 |
| NP_006007.1\| | sialomucin; | | (82%) | (85%) | |
| (NM_006016) | Sialomucin CD164 | | | | |
| | [Homo sapiens] | | | | |
| gi\|13929154\|ref\| | endolyn [Rattus | 195 | 110/197 | 136/197 | 2e−34 |
| NP_114000.1\| | norvegicus] | | (55%) | (68%) | |
| (NM_031812) | | | | | |

The homology of these sequences is shown graphically in the ClustalW analysis shown in Table 25D.

[image]

The sialomucins appear to play 2 key but opposing roles in vivo: the first as cytoprotective or antiadhesive agents, and the second as adhesion receptors. Despite their common functions, these mucins encompass a heterogeneous group of secreted or membrane-associated proteins. See OMIM 603356, SIALOMUCIN or CD164.

Using 2 monoclonal antibodies and a retroviral expression cloning strategy, Zannettino et al. (Zannettino, et al., Blood 92: 2613-2628, 1998, PubMed ID: 9763543) isolated a cDNA encoding a novel transmembrane isoform of the mucin-like glycoprotein MGC-24, which they designated CD164. The mature CD164 protein contains 178 amino acids, has a molecular mass of 80 to 90 kD, and is extremely rich in serine and threonine. CD164 is expressed by human CD34+ hematopoietic progenitor cells. Zannettino et al. (1998) found that the CD164 receptor appears to play a role in hematopoiesis by facilitating the adhesion of CD34+ cells to bone marrow stroma and by negatively regulating CD34+ hematopoietic progenitor cell growth. They found that these functional effects are mediated by at least 2 spatially distinct epitopes, defined by specific monoclonal antibodies. Watt et al. (Watt, et al., Blood 92: 849-866, 1998, PubMed ID: 9680353) showed that these and other CD164 monoclonal antibodies show distinct patterns of reactivity when analyzed on hematopoietic cells from normal human bone marrow, umbilical cord blood, and peripheral blood. Expression of the CD164 epitope was found on developing myelomonocytic cells in bone marrow, being downregulated on mature neutrophils but maintained on monocytes in the peripheral blood. Watt et al. (1998) extended these studies further to identify PAC clones containing the CD164 gene and used the clone to localize the CD164 gene specifically to 6q21 by fluorescence in situ hybridization.

Endolyn is a membrane protein found in lysosomal and endosomal compartments of mammalian cells. Unlike ‘classical’ lysosomal membrane proteins, such as lysosome-associated membrane protein (lamp)-1, it is also present in a subapical compartment in polarized WIF-B hepatocytes. The structural features that determine sorting of endolyn are unknown (1). Ihrke et al. have identified a rat endolyn cDNA by expression screening. The cDNA encodes a ubiquitously expressed type I membrane protein with a short cytoplasmic tail of 13 amino acids and many putative sites for N- and O-linked glycosylation in the predicted luminal domain. Endolyn is closely related to two human mucin-like proteins, multi-glycosylated core protein (MGC)-24 and CD164 (MGC-24v), expressed in gastric carcinoma cells and bone marrow stromal and haematopoietic precursor cells respectively. The predicted transmembrane and cytoplasmic tail domains of endolyn, as well as parts of its luminal domain, also show some similarities with lamp-1 and lamp-2. Like these and other known lysosomal membrane proteins, endolyn contains a YXXO motif at the C-terminus of its cytoplasmic tail (where O is a bulky hydrophobic amino acid), but with no preceding glycine. Nonetheless, the last ten amino acids of this tail, when transplanted on to human CD8, caused efficient targeting of the chimaeric protein to endosomes and lysosomes in transfected normal rat kidney cells (1).

Karlsson et al. demonstrated a genetically determined polymorphism of a human urinary mucin by the separation technique of SDS polyacrylamide gel electrophoresis followed by detection with radioiodinated lectins (2). Peanut agglutinin was the most effective lectin; hence, the proposed designation peanut-reactive urinary mucin (PUM). Karlsson et al. identified 4 common alleles with codominant inheritance. The same polymorphic protein is expressed in other normal and malignant tissues of epithelial origin including the mammary gland. Variation in white cell DNA detected with a cDNA probe for mammary mucin exactly matches the variation of the protein as demonstrated after electrophoresis using a series of monoclonal antibodies; studies in 2 large families demonstrated the precise correspondence. Gendler et al. studied the polymorphic epithelial mucin present on the surface of human mammary cells. It is developmentally regulated and aberrantly expressed in breast cancer (3). Lan et al. used a monospecific polyclonal antiserum against deglycosylated human pancreatic tumor mucin to select clones from a cDNA library developed from a human pancreatic tumor cell line (4). The close similarity of the cDNA sequence and the deduced amino acid sequence of pancreatic mucin to those of breast tumor mucin, as reported by Gendler et al. (3) and others, led them to suggest that the core protein, the apomucin, is produced by the same gene. The native forms of these molecules are distinct in size and degree of glycosylation, however, suggesting that factors other than the primary structure of the apomucin determine these characteristics.

The novel human endolyn-like Proteins of the invention shares 76% homology to the rat Endolyn and to human Mucin CD164. Therefore it is anticipated that this novel protein has a role in the regulation of essentially all cellular functions and could be a potentially important target for drugs. Such drugs may have important therapeutic applications, such as treating numerous tumors. Ibrke et al., Biochem J Jan. 15, 2000;345 Pt2:287-96; Karlsson, et al., Ann. Hum. Genet. 47: 263-269, 1983; Gendler, et al., J. Biol. Chem. 265: 15286-15293, 1990; Lan, Met al., J. Biol. Chem. 265: 15294-15299, 1990.

The protein similarity information, expression pattern, cellular localization, and map location for the NOV25 protein and nucleic acid disclosed herein suggest that this Endolyn-like protein may have important structural and/or physiological functions characteristic of the Mucin family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV25 nucleic acids and proteins of the invention have applications in the diagnosis and/or treatment of various diseases and disorders. For example, the compositions of the present invention will have efficacy for the treatment of patients suffering from: diabetes, Von Hippel-Lindau (VHL) syndrome, pancreatitis, obesity, fertility, hypogonadism, systemic lupus erythematosus, autoimmune disease, asthma, emphysema, scleroderma, allergy, ARDS, psoriasis, actinic keratosis, tuberous sclerosis, acne, hair growth/loss, allopecia, pigmentation disorders, endocrine disorders, renal artery stenosis, interstitial nephritis, glomerulonephritis, polycystic kidney disease, renal tubular acidosis, IgA nephropathy, hypercalceimia, Lesch-Nyhan syndrome as well as other diseases, disorders and conditions.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV25 protein has multiple hydrophilic regions, each of which can be used as an immunogen. In one embodiment, a contemplated NOV25 epitope is from about amino acids 25 to 35. In another embodiment, a contemplated NOV25 epitope is from about amino acids 43 to 62. In other specific embodiments, contemplated NOV25 epitopes are from about amino acids 80 to 110, 125 to 150 and 182 to 187.

NOV26

A disclosed NOV26 (designated CuraGen Acc. No. CG57224-01), which encodes a novel Arylacetamide Deacetylase-like protein and includes the 2082 nucleotide sequence (SEQ ID NO:79) is shown in Table 26A. An open reading frame for the mature protein was identified beginning with an ATG initiation codon at nucleotides 499-501 and ending with a TGA stop codon at nucleotides 1729-1731. Putative untranslated regions are underlined in Table 26A, and the start and stop codons are in bold letters.

TABLE 26A
NOV26 Nucleotide Sequence

(SEQ ID NO:79)

CAGCTTCCCCATGGATCACTCTCCAAATAGATTCTTTACACACAGGTAATGTCACTCAGCCCTTTGGGTCCAACC
CCTTGTCCCCCAGCCCCCGAGTGGTGCTCTTCGGGGGCCCTCATCCATTGGCAAGTGACTGTCTATTCACATCTC
TCTTCCTGTTGTTGAGTGAGTGAGGGAGGGAGCCTGCCGGGGATCCACAGCTCCCAGTTTCCACTCACTCATTAC
ACAGTGCTCTTGGCCCTGCATGTGCTGTCACGGCCATTTGGGGTCTATATCCTGTCTCTTAGAGGACAGGGACTA
AATCTCTCAAATTCAGGTTTCTCCTGTGTCCCTACCTGGTGCCCGGCCCGGGCTGTTTTTCTCTGTTTCAAATGC
CAGGGCTACTTATGGACTCCTATTCAACCTGCAAAACCCTACTTGAATGCTCCCTCAGTTCTGAAGCCTCCCTGG
CTGCTCCTTCCAGCCTCCCCACAACAACAACAGCACCACCACTATATAATGGCTAAATCTGTTGAGCAGTTGCCA
TGGGCCAGACACTGTGCTGAGTACATGGATATGTTTTCTTCTTTAATCCTCACAACCCCTCGAGTCAGCCCCAAG
CTAGGCTACCCTTTGGCAAATTCACATCATTATTCAATCAAGAGCCTCTGGGGAGAAAAGTTGGAAAACCCAGCC
CTCTACCTGGACACAGTCCAGAGCCTATGGATTCCTGAAGAGCCCCCTGTACCTACAGGAGGCAGCGTGAGAATT
AAAAAGGACCCTGAACTTGTGGTGACCGACCTGCGTTTTGGGACGATACCCGTGAGGCTGTTCCAGCCGAAGGCA
GCATCCTCCAGACCCCGGCGAGGCATCATCTTCTACCATGGAGGGGCCACAGTATTTGGGAGCCTGGATTGTTAC
CATGGCCTGTGCAATTATCTGGCCCGGGAGACTGAATCTGTACTTCTGATGATTGGGTACCGCAAGCTTCCTGAC
CACCATTCCCCTGCCCTTTTCCAAGACTGCATGAATGCCTCCATTCACTTCCTGAAGGCCCTGGAAACCTATGGG
GTGGACCCCTCCAGGGTTGTGGTCTGTGGAGAAAGCGTCGGAGGTGCAGCGGTGGCCGCCATCACCCAGGCCTTG
GTGGGCAGATCAGATCTTCCCCGGATCCGGGCTCAGGTTCTGATTTATCCAGTTGTCCAGGCATTCTGTTTGCAG
TCGCCATCCTTTCAGCAGAACCAAAATGTCCCATTACTTTCCCGGAAGTTCATGGTGACTTCTCTGTGTAACTAT
CTGGCCATTGACCTCTCCTGGCGTGACGCCATCTTGAACGGCACTTGCGTACCCCCAGACGTCTGGAGGAAGTAC
GAGAAGTGGCTCACCCCTGACAACATCCCCAAGAAATTTAAGAACACAGGCTACCAACCCTGGTCTCCCGGCCCT
TTTAATGAAGCTGCCTATCTAGAAGCCAAACATATGCTGGATGTAGAAAATTCACCCCTGATAGCAGATGATGAG
GTCATCGCTCAGCTTCCTGAGGCCTTCCTGGTGAGCTGTGAGAATGACATACTCCGTGATGACAGCTTGCTCTAT
AAGAAGCGCTTGGAGGACCAGGGGGTCCGCGTGACATGGTACCACCTGTATGATGGTTTTCACGGATCCATTATC
TTTTTTGATAAGAAGGCTCTCTCTTTCCCATGTTCCCTGAAGATTGTGAATGCTGTAGTCAGTTATATAAAGGGC
ATATGATAGTAACCCTGGGGCCCGAGGAGGAAGGGGCAAGTATGGACTCTACCAGAAACCGGGTGCTTTAGTGAG
TTCTATTTTATTGACTAAAGAGGTGCTACATCAATGCTTGGGGCAGCTGGGAAGGGTGAGAAGTAAGCTAACAGT
CTTGCTTAGTATTCAAGAAAATCCAAACTGTGTCTGTTTCCTTCCAGCACTAACAATGTCCATTGCTGGATCTAG
CGACATTCTCTAACATTCCCATTTAGGTGAAATAAATATCAAAAGGAGAAAAAAATGCCTTTAAAAATTTCTCAA
AGCCCCAACATATAAGATCTGTGCAGAATAAATGCCAACAACTGGTCATACCGTCAA

The disclosed NOV26 nucleic acid has 295 of 500 bases (59%) identical to a gb:GENBANK-ID:AB037784|acc:AB037784.1 mRNA from Homo sapiens (Homo sapiens mRNA for KIAA1363 protein, partial cds) (E=2.3e⁻⁰⁸).

A disclosed NOV26 polypeptide (SEQ ID NO:80) is 410 amino acid residues in length and is presented using the one-letter amino acid code in Table 26B. The SignalP, Psort and/or Hydropathy results predict that NOV26 does not have a signal peptide and is likely to be localized to the nucleus with a certainty of 0.8800. In alternative embodiments, a NOV26 polypeptide is located to the microbody (peroxisome) with a certainty of 0.2235, the lysosome (membrane) with a certainty of 0.1734, or the mitochondrial matrix space with a certainty of 0.1000.

TABLE 26B
Encoded NOV26 Protein Sequence

(SEQ ID NO:80)

MAKSVEQLPWARHCAEYMDMFSSLILTTPRVSPKLGYPLANSHHYSIKSLWGEKLENPALYLDTVQSLWIPEEPPV
PTGGSVRIKKDPELVVTDLRFGTIPVRLFQPKAASSRPRRGIIFYHGGATVFGSLDCYHGLCNYLARETESVLLMI
GYRKLPDHHSPALFQDCMNASIHFLKALETYGVDPSRVVVCGESVGGAAVAAITQALVGRSDLPRIRAQVLIYPVV
QAFCLQSPSFQQNQNVPLLSRKFMVTSLCNYLAIDLSWRDAILNGTCVPPDVWRKYEKWLTPDNIPKKFKNTGYQP
WSPGPFNEAAYLEAKHMLDVENSPLIADDEVIAQLPEAFLVSCENDILRDDSLLYKKRLEDQGVRVTWYHLYDGFH
GSIIFFDKKALSFPCSLKIVNAVVSYIKGI

The NOV26 amino acid sequence was found to have 116 of 325 amino acid residues (35%) identical to, and 183 of 325 amino acid residues (56%) similar to, the 398 amino acid residue ptnr:TREMBLNEW-ACC:AAG60035 protein from Mus musculus (Mouse) (ARYLACETAMIDE DEACETYLASE) (E=5.4e⁻⁴⁷).

NOV26 is expressed in at least the following tissues: Pooled human melanocyte, fetal heart, and pregnant uterus. Expression information was derived from the tissue sources of the sequences that were included in the derivation of the sequence of CuraGen Acc. No. CG57224-01. The sequence is predicted to be expressed in the brain because of the expression pattern of (GENBANK-ID: gb:GENBANK-ID:AB037784|acc:AB037784.1), a closely related Homo sapiens mRNA for KIAA1363 protein, partial cds homolog in species Homo sapiens.

Homologies to the above NOV26 proteins will be shared by the other NOV26 proteins insofar as they are homologous to each other. Any reference to NOV26 is assumed to refer to NOV26 proteins in general.

NOV26 has homology to the amino acid sequences shown in the BLASTP data listed in Table 26C.

TABLE 26C
BLAST results for NOV26

| Gene Index/ | | Length | Identity | Positives | |
|---|---|---|---|---|---|
| Identifier | Protein/Organism | (aa) | (%) | (%) | Expect |
| gi\|17438979\|ref\| | similar to | 407 | 327/330 | 328/330 | 0.0 |
| XP_060166.1\|(XM_060166) | ARYLACETAMIDE | | (99%) | (99%) | |
| | DEACETYLASE | | | | |
| | (AADAC) (H. | | | | |
| | sapiens) [Homo | | | | |
| | sapiens] | | | | |
| gi\|17438981\|ref\| | similar to | 409 | 185/388 | 244/388 | 2e−94 |
| XP_060167.1\|(XM_060167) | arylacetamide | | (47%) | (62%) | |
| | deacetylase (H. | | | | |
| | sapiens) [Homo | | | | |
| | sapiens] | | | | |
| gi\|7513557\|pir\|\| | esterase/N- | 398 | 117/333 | 179/333 | 2e−46 |
| A58922 | deacetylase (EC | | (35%) | (53%) | |
| | 3.5.1.-), 50K | | | | |
| | hepatic - rabbit | | | | |
| gi\|4557227\|ref\| | arylacetamide | 399 | 127/379 | 200/379 | 8e−46 |
| NP_001077.1\|(NM_001086) | deacetylase [Homo | | (33%) | (52%) | |
| | sapiens] | | | | |
| gi\|10120490\|ref\| | arylacetamide | 398 | 113/330 | 179/330 | 8e−46 |
| NP_065413.1\|(NM_020538) | deacetylase | | (34%) | (54%) | |
| | [Rattus | | | | |
| | norvegicus] | | | | |

The homology of these sequences is shown graphically in the ClustalW analysis shown in Table 26D.

[image]

[image]

Table 126E lists the domain description from DOMAIN analysis results against NOV26. This indicates that the NOV26 sequence has properties similar to those of other proteins known to contain these domains.

TABLE 26E
Domain Analysis of NOV26

gnl|Pfam|pfam00135, COesterase, Carboxylesterase.
CD-Length = 532 residues, 22.2% aligned
Score = 43.5 bits (101), Expect = 2e−05

| | | | | |
|---|---|---|---|---|
| NOV26: | 104 | LFQPKAASSRPRRGIIFY-HGGATVFGS-LDCYHGLCNYLARETESVLLMIGYR------ | 155 | (SEQ ID NO:356) |
| | | ++ \|\| + ++ + \|\|\| +\|\|\| \| \| \| \|\|\|\| +++ \| \|\| | | |
| Sbjct: | 109 | VYTPKNRKPNSKLPVMVMIHGGGFMFGSGLSLYDGE--SLAREGNVIVVSINYRLGPLGF | 166 | (SEQ ID NO:357) |
| NOV26: | 156 | -KLPDHHSPALFQDCMNASIHF-LKALE-------TYGVDPSRVVVCGESVGGAAVAAIT | 206 | |
| | | \| \| \| + \|\|+ +\| \|\| \| + \|\|\| \|\|\|+\|+ + | | |
| Sbjct: | 167 | LSTGDDVLPG------NYGLLDQRLALKWVQDNIAAFGGDPDSVTIFGESAGGASVSLLL | 220 | |
| NOV26: | 207 | QALVGR 212 | | |
| | | + + | | |
| Sbjct: | 221 | LSPSSK 226 | | |

The deacetylation of monoacetyldapsone (MADDS) has been examined in liver microsomes and cytosol from male Sprague-Dawley rats, Golden Syrian hamsters, and Swiss Abino mice. All three rodent species demonstrated greater MADDS deacetylation activity in liver microsomes than in liver cytosol. Further investigations were conducted in hamsters. The velocity of MADDS deacetylation in major organs in the hamster was greatest in the intestine, followed by the liver and kidney. The effect of pretreatment with common inducers on liver microsomal deacetylation activity was also examined in the hamster. Phenobarbital, 100 mg/kg/day×3 days, did not alter activity, while dexamethasone at the same dose reduced 2-acetylaminofluorene (2-AFF), MADDS, and p-n itrophenyl acetate (NPA) hydrolysis by at least 50%. Due to a previous report that KI activated the deacetylation of an arylacetamide in vitro (Khanna et al., J Pharmacol Exp Ther 262:1225-1231, 1992), the effects of the halides KF, KCl, KBr and KI on MADDS hydrolysis in vitro were tested. Of the halides studied, only KF altered MADDS hydrolysis, resulting in an almost complete inhibition of deacetylase activity at 50 mM (with the initial concentration of MADDS at 0.6 mM) with an IC50=0.16 mM. Cornish-Bowden and Dixon plots indicated that the inhibition exerted by KF was non-competitive. The rank order of inhibitor potencies was constructed using phenylmethylsulfonyl fluoride (PMSF), bis(p-nitrophenyl)phosphate (BNPP), physostigmine, and KF with 2-AFF, MADDS, and NPA as substrates. Different rank order potencies were obtained for each of the substrates tested. The substrates 2-AFF, MADDS, and NPA did not act as competitive inhibitors on the hydrolysis rates of each other. Liver microsomal arylacetamide deacetylase activity was greater in male hamsters than in females with either MADDS or 2-AAF as substrates; however, hydrolysis of NPA was similar in both male and female hamsters. These data support the hypothesis that the enzyme which catalyzes the hydrolysis of MADDS differs from that catalyzing either 2-AAF or NPA hydrolysis.

The relative ability of arylacetamide deacetylase enzyme systems of dog liver to carry out the deacetylation of the carcinogens, 4-acetylaminobiphenyl, 2-acetylaminofluorene, and 2-acetylaminaphthalene, was examined. The arylacetamides were incubated with unfortified dog liver microsomes, and enzyme activity (nmol arylamine/mg protein/hr) was estimated by colorimetric quantitation of the resulting arylamines. The dog liver enzyme system displayed characteristics similar to those described for the rodent liver enzyme system in that enzyme activity was greatest in liver tissue, was localized in the microsomal subcellular fraction, required no cofactors, and was inhibited by heat, sodium fluoride, and thiol reagents. In five replicate assays, the relative rates of deacetylation were about 10, 6, and 1 with 4-acetylaminobiphenyl (84.8+/−12.4), 2-acetylaminofluorene (52.5+/−5.1), and 2-acetylaminonaphthalene (8.8+/−3.3), respectively. As a canine urinary bladder carcinogen, 4-acetylaminobiphenyl is considered more potent than 2-acetylaminofluroene, while 2-acetylaminonaphthalene is devoid of detectable carcinogenic activity, despite the fact that 2-aminoaphthalene is a well-established canine urinary bladder carcinogen. Removal of the acetyl group may be a requirement for urinary bladder carcinogenesis; accordingly, the present studies demonstrate the appearance of a direct relationship between dog liver deacetylase enzyme specificity and urinary bladder susceptibility to these carcinogenic arylacetamides.

The protein similarity information, expression pattern, cellular localization, and map location for the NOV26 protein and nucleic acid disclosed herein suggest that this Arlyacetamide Deacetylase-like protein may have important structural and/or physiological functions characteristic of the Protease family. Therefore, the nucleic acids and proteins of the invention are useflul in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV26 nucleic acids and proteins of the invention have applications in the diagnosis and/or treatment of various diseases and disorders. For example, the compositions of the present invention will have efficacy for the treatment of patients suffering from: Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection as well as other diseases, disorders and conditions.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV26 protein has multiple hydrophilic regions, each of which can be used as an immunogen. In one embodiment, a contemplated NOV26 epitope is from about amino acids 5 to 10. In another embodiment, a contemplated NOV26 epitope is from about amino acids 40 to 55. In other specific embodiments, contemplated NOV26 epitopes are from about amino acids 60 to 85, 105 to 120, 140 to 142, 155 to 162, 240 to 252, 260 to 340 and 350 to 380.

NOV27

A disclosed NOV27 (designated CuraGen Acc. No. CG57288-01), which encodes a novel Olfactory Receptor-like protein and includes the 1008 nucleotide sequence (SEQ ID NO:81) is shown in Table 27A. An open reading frame for the mature protein was identified beginning with an GCA initiation codon at nucleotides 1-3 and ending with a TGA stop codon at nucleotides 922-924. Putative untranslated regions are underlined in Table 27A, and the start and stop codons are in bold letters.

TABLE 27A
NOV27 Nucleotide Sequence

(SEQ ID NO:81)

GCAGAGGAGCTCCTTGGATTTTCTTATCTCCATGAGTTCCAGGTTCTGCTGTTTGCTCTGATCCTGTTGATATATG
TGCTGATGCTGCTGGGCAACCTGGCCATCATCAGCTTCATTTGCCTTGATTCCCGCCTTCACTCACCCATGTACTT
CTTCCTCTGCAACTTCTCCCTCATGGAGATGGTGGTCACCTCCACTGTGGTACATAGGATGCTGGCAGACCTGCTA
TCCACTCACAAGACCATGTCCCTGGCCAAATGCCTAACCCAGTCTTTCTTTTACTTCTCCCTGGGCTCTGCCAACT
TCCTGATACTCATGGTCATGGCCTTTGATCGCTACGTGGCCATCTGCEACCCCCTGCGCTACCCAACCATCACGAA
TGGTCCAGTGTGTGTGAAGCTGGTGGTGGCCTGTTGGGTGGTTGGTTTCCTCTCCATTGTCTCTCCCACACTGCAG
AAAACACGACTCTGGTTCTGTGGCCCTAACATCATCGGCCACTACTTCTGTGACTCTGCCCCGCTGCTCAAGCTTG
CCTGCTCTGACACCCGCCACATTGAGCGCATGGACCTCTTCCTGTCCCTGCTCTTTGTGCTGACCACCATGCTGCT
TATCATCCTCTCCTACATCCTCATTGTGGCTGCAGTGCTGCACATCCCTTCCTCCTCTGGATGCCAGAAGGCCTTC
TCCACCTGTGCCCCTCACCTCACAGTGGTGGTTCTGGGCTATGGCAGTGCCATCTTCATCTACGTGAGGCCAGGCA
AGGGCCACTCCACATACCTCAACAAGGCGGTGGCCATGGTGACTGCAATGGTAACCCCTTTCCTCAACCCCTTCAT
CTTCACCTTCCGGAATGAGAAGGTCAAGGAGGTCATTGAGGATGTGACTAAAAGGATCTTCCTTGGAGACCCAGCA
GCCTGTAGGTGAGAGGGTGAGCCCTTGACAGGGCTAGAGAGCACCTGACAAGTCACGAGGAGTAGACTTGCTGCAG
GTGGGCACCCACATGCCTAA

The disclosed NOV27 nucleic acid has 540 of 892 bases (60%) identical to a gb:GENBANK-ID:AP002533|acc:AP002533.1 mRNA from Homo sapiens (Homo sapiens genomic DNA, chromosome 1q22-q23, CD1 region, section 2/4) (E=1.8e⁻³⁷).

The NOV27 polypeptide (SEQ ID NO:82) is 307 amino acid residues in length and is presented using the one-letter amino acid code in Table 27B. The SignalP, Psort and/or Hydropathy results predict that NOV27 has a signal peptide and is likely to be localized to the endoplasmic reticulum (membrane) with a certainty of 0.6850. In alternative embodiments, a NOV27 polypeptide is located to the plasma membrane with a certainty of 0.6400, the Golgi body with a certainty of 0.4600, or the endoplasmic reticulum (lumen) with a certainty of 0.1000. The SignalP predicts a likely cleavage site for a NOV27 peptide between amino acid positions 34 and 35, i.e. at the sequence NLA-II.

TABLE 27B
Encoded NOV27 Protein Sequence

(SEQ ID NO:82)

AEELLGFSYLHEFQVLLFALILLIYVLMLLGNLAIISFICLDSRLHSPMYFFLCNFSLMEMVVTSTVVHRMLAD
LLSTHKTMSLAKCLTQSFFYFSLGSANFLILMVMAFDRYVAICHPLRYPTITNGPVCVKLVVACWVVGFLSIVS
PTLQKTRLWFCGPNIIGHYFCDSAPLLKLACSDTRHIERMDLFLSLLFVLTTMLLIILSYILIVAAVLHIPSSS
GCQKAFSTCAPHLTVVVLGYGSAIFIYVRPGKGHSTYLNKAVAMVTAMVTPFLNPFIFTFRNEKVKEVIEDVTK
RIFLGDPAACR

The NOV27 amino acid sequence was found to have 143 of 295 amino acid residues (48%) identical to, and 198 of 295 amino acid residues (67%) similar to, the 313 amino acid residue ptnr:SPTREMBL-ACC:Q9Z1V0 protein from Mus musculus (Mouse) (OLFACTORY RECEPTOR C6) (E=1.1e⁻⁶⁹).

NOV27 is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

Possible small nucleotide polymorphisms (SNPs) found for NOV27 are listed in Table 27C.

TABLE 27C

SNPs

| | | | | |
|---|---|---|---|---|
| | Nucleotide | Base | Amino Acid | Base |
| Variant | Position | Change | Position | Change |

| | | | | |
|---|---|---|---|---|
| 13377027 | 620 | C > A | 207 | Pro > His |

Homologies to any of the above NOV27 proteins will be shared by the other NOV27 proteins insofar as they are homologous to each other as shown above. Any reference to NOV27 is assumed to refer to both of the NOV27 proteins in general, unless otherwise noted.

NOV27 also has homology to the amino acid sequences shown in the BLASTP data listed in Table 27D.

TABLE 27D
BLAST results for NOV27

| Gene Index/ | | Length | Identity | Positives | |
|---|---|---|---|---|---|
| Identifier | Protein/Organism | (aa) | (%) | (%) | Expect |
| gi\|15723374\|ref\| | olfactory receptor | 280 | 279/280 | 279/280 | e−134 |
| NP_277054.1\|(NM_033519) | sdolf [Homo | | (99%) | (99%) | |
| | sapiens] | | | | |
| gi\|15293799\|gb\| | olfactory receptor | 216 | 215/216 | 215/216 | 2e−98 |
| AAK95092.1\|(AF399607) | [Homo sapiens] | | (99%) | (99%) | |
| gi\|17476501\|ref\| | similar to | 1056 | 145/295 | 210/295 | 4e−80 |
| XP_063251.1\|(XM_063251) | OLFACTORY | | (49%) | (71%) | |
| | RECEPTOR-LIKE | | | | |
| | PROTEIN F6 (H. | | | | |
| | sapiens) [Homo | | | | |
| | sapiens] | | | | |
| gi\|17464943\|ref\| | similar to | 313 | 155/295 | 210/295 | 3e−74 |
| XP_069610.1\|(XM_069610) | olfactory receptor | | (52%) | (70%) | |
| | sdolf (H. sapiens) | | | | |
| | [Homo sapiens] | | | | |
| gi\|17476599\|ref\| | similar to | 347 | 149/295 | 207/295 | 3e−64 |
| XP_063285.1\|(XM_063285) | olfactory receptor | | (50%) | (69%) | |
| | sdolf (H. sapiens) | | | | |
| | [Homo sapiens] | | | | |

The homology of these sequences is shown graphically in the ClustalW analysis shown in Table 27E.

[image]

[image]

[image]

Table 27F lists the domain description from DOMAIN analysis results against NOV27. This indicates that the NOV27 sequence has properties similar to those of other proteins known to contain the 7 transmembrane receptor domain.

TABLE 27F
Domain Analysis of NOV27

gnl|Pfam|pfam00001, 7tm_1, 7 transmembrane receptor (rhodopsin family).
CD-Length = 254 residues, 98.4% aligned
Score = 73.2 bits (178), Expect = 2e−14

| | | | | |
|---|---|---|---|---|
| NOV27: | 35 | IISFICLDSRLHSPMYFFLCNFSLMEMVVTSTVVHRMLADLLSTHKTMSLAKCLTQSFFY | 94 | (SEQ ID NO:363) |
| | | +\| \| +\| +\| \|\| \| ++ +++ \|+ \| \|+ \| \| + | | |
| Sbjct: | 5 | VILVILRTKKLRTPTNIFLLNLAVADLLFLLTLPPWALYYLVGGDWVFGDALCKLVGALF | 64 | (SEQ ID NO:364) |
| NOV27: | 95 | FSLGSANFLILMVMAFDRYVAICHPLRYPTITNGPVCVKLVVACWVVGFLSIVSPTLQKT | 154 | |
| | | \| \|+ \|+\| ++ \|\|\|+\|\| \|\|\|\|\| \| \|++ \|\|+ \| + \| \| | | |
| Sbjct: | 65 | VVNGYASILLLTAISIDRYLAIVHPLRYRRIRTPRRAKVLILLVWVLALLLSLPPLLFSW | 124 | |
| NOV27: | 155 | RLWFCGPNIIGHYFCDSAPLLKLACSDTRHIERMDLFLSLLFVLTTMLLIILSYILIVAA | 214 | |
| | | \| +\| + + \| \| ++ \| \| +\| \| | | |
| Sbjct: | 125 | LRTVEEGNTTVCLIDFPEESVKRSYVLLSTLVGFVLPLLVILVCYTRILRTLRKRARSQR | 184 | |
| NOV27: | 215 | VLHIPSSSGCQKAFSTCAPHLTVVVLGYGSAIFIYVRP----GKGHSTYLNKAVAMVTAM | 270 | |
| | | \| \|\|\| + \| + \|+ \| + + + + \| | | |
| Sbjct: | 185 | SLKRRSSSERKAAKMLLVVVVVFVLCWLPYHIVLLLDSLCLLSIWRVLPTALLITLWLAY | 244 | |
| NOV27: | 271 | VTPFLNPFIF | 280 | |
| | | \| \|\|\| \|+ | | |
| Sbjct: | 245 | VNSCLNPIIY | 254 | |

G-Protein Coupled Receptor (GPCRs) have been identified as an extremely large family of protein receptors in a number of species. At the phylogenetic level they can be classified into four major subfamilies. These receptors share a seven transmembrane domain structure with many neurotransmitter and hormone receptors. They are likely to be involved in the recognition and transduction of various signals mediated by G-Proteins, hence their name G-Protein Coupled Receptors. The human GPCR genes are generally intron-less and belong to four gene subfamilies, displaying great sequence variability. These genes are dominantly expressed in olfactory epithelium.

Olfactory receptors (ORs) have been identified as extremely large family of GPCRs in a number of species. As members of the GPCR family, these receptors share a seven transmembrane domain structure with many neurotransmitter and hormone receptors, and are likely to underlie the recognition and G-protein-mediated transduction of odorant signals. Like GPCRs, the ORs they can be expressed in a variety of tissues where they are thought to be involved in recognition and transmission of a variety of signals. The human OR genes are typically intron-less and belong to four different gene subfamilies, displaying great sequence variability. These genes are dominantly expressed in olfactory epithelium.

The protein similarity information, expression pattern, cellular localization, and map location for the NOV27 protein and nucleic acid disclosed herein suggest that this Olfactory Receptor-like protein may have important structural and/or physiological functions characteristic of the Olfactory Receptor family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV27 nucleic acids and proteins of the invention are useful in potential diagnostic and therapeutic applications implicated in various diseases and disorders described below and/or other pathologies. For example, the compositions of the present invention will have efficacy for treatment of patients suffering from: developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders; control of feeding; potential obesity due to over-eating; potential disorders due to starvation (lack of appetite), non-insulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome and/or other pathologies and disorders of the like. The polypeptides can be used as immunogens to produce antibodies specific for the invention, and as vaccines. They can also be used to screen for potential agonist and antagonist compounds. For example, a cDNA encoding the OR-like protein may be useful in gene therapy, and the OR-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crobn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding OR-like protein, and the OR-like protein of the invention, or fragments thereof, may further be useful in diagnostic applications, wherein the presence or amount of the nucleic acid or the protein are to be assessed.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV27 protein has multiple hydrophilic regions, each of which can be used as an immunogen. In one embodiment, a contemplated NOV27 epitope is from about amino acids 45 to 55. In another embodiment, a contemplated NOV27 epitope is from about amino acids 75 to 95. In other specific embodiments, contemplated NOV27 epitopes are from about amino acids 110 to 140, 150 to 180, 210 to 240, 250 to 265 and 270 to 295.

NOV28

A disclosed NOV28 (designated CuraGen Acc. No. CG57213-01), which encodes a novel PB39-like protein and includes the 2233 nucleotide sequence (SEQ ID NO:83) is shown in Table 28A. An open reading frame for the mature protein was identified beginning with an ATG initiation codon at nucleotides 77-79 and ending with a TAG stop codon at nucleotides 1661-1663. Putative untranslated regions are underlined in Table 28A, and the start and stop codons are in bold letters.

TABLE 28A

NOV28 Nucleotide Sequence

(SEQ ID NO:83)

CCGGGGCTGGAGGGGGGCAAGCGGGTTCCGAGGTGCAAAGCCTGGTGCCCCGAGCCCTGCGGAGCTCGGGGCCA
GCATGGCCCCCACGCTGCAACAGGCGTACCGGAGGCGCTGGTGGATGGCCTGCACGGCTGTGCTGGAGAACCTC
TTCTTCTCTGCTGTACTCCTGGGCTGGGGCTCCCTGTTGATCATTCTGAAGAACGAGGGCTTCTATTCCAGCAC
GTGCCCAGCTGAGAGCAGCACCAACACCACCCAGGATGAGCAGCGCAGGTGGCCAGGCTGTGACCAGCAGGACG
AGATGCTCAACCTGGGCTTCACCATTGGTTCCTTCGTGCTCAGCGCCACCACCCTGCCACTGGGGATCCTCATG
GACCGCTTTGGCCCCCGACCCGTGCGGCTGGTTGGCAGTGCCTGCTTCACTGCGTCCTGCACCCTCATGGCCCT
GGCCTCCCGGGACGTGGAAGCTCTGTCTCCGTTGATATTCCTGGCGCTGTCCCTGAATGGCTTTGGTGGCATCT
GCCTAACGTTCACTTCACTCAAGCTGATCTACGATGCCGGTGTGGCCTTCGTGGTCATCATGTTCACCTGGTCT
GGCCTGGCCTGCCTTATCTTTCTGAACTGCACCCTCAACTGGCCCATCGAAGCCTTTCCTGCCCCTGAGGAAGT
CAATTACACGAAGAAGATCAAGCTGAGTGGGCTGGCCCTGGACCACAAGGTGACAGGTGACCTCTTCTACACCC
ATGTGACCACCATGGGCCAGAGGCTCAGCCAGAAGGCCCCCAGCCTGGAGGACGGTTCGGATGCCTTCATGTCA
CCCCAGGATGTTCGGGGCACCTCAGAAAACCTTCCTGAGAGGTCTGTCCCCTTACGCAAGAGCCTCTGCTCCCC
CACTTTCCTGTGGAGCCTCCTCACCATGTGCATGACCCAGCTGCGGATCATCTTCTACATGGCTGCTGTGAACA
AGATGCTGGAGTACCTTGTGACTGGTGGCCAGGAGCATGAGACAAATGAACAGCAACAAAAGGTGGCAGAGACA
GTTGGGTTCTACTCCTCCGTCTTCGGGGCCATGCAGCTGTTGTGCCTTCTCACCTGCCCCCTCATTGGCTACAT
CATGGACTGGCGGATCAAGGACTGCGTGGACGCCCCAACTCAGGGCACTGTCCTCGGAGATGCCAGGGACGGGG
TTGCTACCAAATCCATCAGACCACGCTACTGCAAGATCCAAAAGCTCACCAATGCCATCAGTGCCTTCACCCTG
ACCAACCTGCTGCTTGTGGGTTTTGGCATCACCTGTCTCATCAACAACTTACACCTCCAGTTTGTGACCTTTGT
CCTGCACACCATTGTTCGAGGTTTCTTCCACTCAGCCTGTGGGAGTCTCTATGCTGCAGTGTTCCCATCCAACC
ACTTTGGGACGCTGACAGGCCTGCAGTCCCTCATCAGTGCTGTGTTCGCCTTGCTTCAGCAGCCACTTTTCATG
GCGATGGTGGGACCCCTGAAAGGAGAGCCCTTCTGGGTGAATCTGGGCCTCCTGCTATTCTCACTCCTGGGATT
CCTGTTGCCTTCCTACCTCTTCTATTACCGTGCCCGGCTCCAGCAGGAGTACGCCGCCAATGGGATGGGCCCAC
TGAAGGTGCTTAGCGGCTCTGAGGTGACCGCATAGACTTCTCAGACCAAGGGACCTGGATGACAGGCAATCAAG
GCCTGAGCAACCAAAAGGAGTGCCCCATATGGCTTTTCTACCTGTAACATGCACATAGAGCCATGGCCGTAGAT
TTATAAATACCAAGAGAAGTTCTATTTTTGTAAAGACTGCAAAAAGGAGGAAAAAAAACCTTCAAAAACGCCCC
CTAAGTCAACGCTCCATTGACTGAAGACAGTCCCTATCCTAGAGGGGTTGAGCTTTCTTCCTCCTTGGGTTGGA
GGAGACCAGGGTGCCTCTTATCTCCTTCTAGCGGTCTGCCTCCTGGTACCTCTTGGGGGGATCGGCAAACAGGC
TACCCCTGAGGTCCCATGTGCCATGAGTGTGCACAACATGCAATGTGTCTGTGTATGTGTGAATGTGAGAAAAA
CACAGCCCTCCTTTCAGAAGGAAAGGGGCCTGAGGTGCCAGCTGTGTCCTGGGTTAGGGGTTGGGGGTCGGCCC
CTTCCAGGGCCAGGAAGGCAGGTTCCCTCTCTGGTGCTGCTGCTTGCAAGTCTTAGAGGAAATAAAAAGGGAAG
TGAGAAAAAAAAA

The disclosed NOV28 nucleic acid has been mapped to chromosome 11p11.2-p11.1 and has 1866 of 1993 bases (93%) identical to a gb:GENBANK-ID:AF045584|acc:AF045584.1 mRNA from Homo sapiens (Homo sapiens PB39 mRNA, complete cds) (E=0.0).

The NOV28 polypeptide (SEQ ID NO:84) is 528 amino acid residues in length and is presented using the one-letter amino acid code in Table 28B. The SignalP, Psort and/or Hydropathy results predict that NOV28 has a signal peptide and is likely to be localized to the mitochondrial inner membrane with a certainty of 0.6450. In alternative embodiments, a NOV28 polypeptide is located to the plasma membrane with a certainty of 0.6000, the mitochondrial intermembrane space with a certainty of 0.5634, or the mitochondrial matrix space with a certainty of 0.4367. The SignalP predicts a likely cleavage site for a NOV28 peptide between amino acid positions 44 and 45, i.e. at the sequence NEG-FY.

TABLE 28B

Encoded NOV28 Protein Sequence

(SEQ ID NO:84)

MAPTLQQAYRRRWWMACTAVLENLFFSAVLLGWGSLLIILKNEGFYSSTCPAESSTNTTQDEQRRWPGCDQQDEMLN
LGFTIGSFVLSATTLPLGILMDRFGPRPVRLVGSACFTASCTLMALASRDVEALSPLIFLALSLNGFGGICLTFTSL
KLIYDAGVAFVVIMFTWSGLACLIFLNCTLNWPIEAFPAPEEVNYTKKIKLSGLALDHKVTGDLFYTHVTTMGQRLS
QKAPSLEDGSDAFMSPQDVRGTSENLPERSVPLRKSLCSPTFLWSLLTMCMTQLRIIFYMAAVNKMLEYLVTGGQEH
ETNEQQQKVAETVGFYSSVFGAMQLLCLLTCPLIGYIMDWRIKDCVDAPTQGTVLGDARDGVATKSIRPRYCKIQKL
TNAISAFTLTNLLLVGFGITCLINNLHLQFVTFVLHTIVRGFFHSACGSLYAAVFPSNHFGTLTGLQSLISAVFALL
QQPLFMAMVGPLKGEPFWVNLGLLLFSLLGFLLPSYLFYYRARLQQEYAANGMGPLKVLSGSEVTA

The NOV28 amino acid sequence was found to have 384 of 419 amino acid residues (91%) identical to, and 391 of 419 amino acid residues (93%) similar to, the 559 amino acid residue ptnr:SPTREMBL-ACC:075387 protein from Homo sapiens (Human) (PB39) (E=9.3e⁻²⁸⁶).

NOV28 is expressed in at least the following tissues: adrenal gland, bone marrow, brain—amygdala, brain—cerebellum, brain—hippocampus, brain—substantia nigra, brain—thalamus, brain-whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus, Liver, Lymphoid tissue, Tonsils, and Whole Organism. Expression information was derived from the tissue sources of the sequences that were included in the derivation of the sequence of NOV28. The sequence is predicted to be expressed in prostate epithelium because of the expression pattern of (GENBANK-ID: gb:GENBANK-ID:AF045584|acc:AF045584.1), a closely related Homo sapiens PB39 mRNA, complete cds hololog.

Possible small nucleotide polymorphisms (SNPs) found for NOV28 are listed in Tables 28C and 28D.

TABLE 28C
SNPs

| Consensus Position | Depth | Base Change | PAF |
|---|---|---|---|
| 22 | 8 | C > A | 0.250 |
| 408 | 4 | G > T | 0.500 |
| 418 | 4 | G > T | 0.500 |
| 427 | 4 | G > T | 0.500 |
| 454 | 4 | A > T | 0.500 |
| 455 | 4 | G > C | 0.500 |
| 458 | 4 | G > C | 0.500 |
| 495 | 4 | G > C | 0.500 |

TABLE 28D
SNPs

| | Nucleotide | | Amino Acid | |
|---|---|---|---|---|
| Variant | Position | Base Change | Position | Base Change |
| 13377029 | 1488 | T > C | 471 | Val > Ala |

Homologies to any of the above NOV28 proteins will be shared by the other NOV28 proteins insofar as they are homologous to each other as shown above. Any reference to NOV28 is assumed to refer to both of the NOV28 proteins in general, unless otherwise noted.

NOV28 also has homology to the amino acid sequences shown in the BLASTP data listed in Table 28E.

TABLE 28E
BLAST results for NOV28

| Gene Index/ | | Length | Identity | Positives | |
|---|---|---|---|---|---|
| Identifier | Protein/Organism | (aa) | (%) | (%) | Expect |
| gi\|4505971\|ref\| | prostate cancer | 559 | 527/559 | 527/559 | 0.0 |
| NP_003618.1\|(NM_003627) | overexpressed gene | | (94%) | (94%) | |
| | 1 [Homo sapiens] | | | | |
| gi\|12847527\|dbj\| | data source: MGD, | 654 | 426/552 | 466/552 | 0.0 |
| BAB27605.1\|(AK011417) | source | | (77%) | (84%) | |
| | key: MGI: 1931352, | | | | |
| | evidence: ISS˜ | | | | |
| | prostate cancer | | | | |
| | overexpressed gene | | | | |
| | 1˜putative [Mus | | | | |
| | musculus] | | | | |
| gi\|15310953\|ref\| | prostate cancer | 401 | 377/392 | 382/392 | 0.0 |
| XP_046257.2\|(XM_046257) | overexpressed gene | | (96%) | (97%) | |
| | 1 [Homo sapiens] | | | | |
| gi\|18027388\|gb\| | unknown [Homo | 489 | 205/407 | 263/407 | e−102 |
| AAL55776.1\|AF289592_1 (AF289592) | sapiens] | | (50%) | (64%) | |
| gi\|18042965\|gb\| | Unknown (protein | 373 | 198/359 | 257/359 | 6e−99 |
| AAH19562.1\|AAH19562 (BC019562) | for IMAGE: 3451144) | | (55%) | (71%) | |
| | [Homo sapiens] | | | | |

The homology of these sequences is shown graphically in the ClustalW analysis shown in Table 28F.

[image]

[image]

[image]

The gene PB39 (HGMW-approved symbol POV1), whose expression is up-regulated in human prostate cancer, has been identified using tissue microdissection-based differential display analysis. The full-length sequence of PB39 cDNA, the genomic localization of the PB39 gene, and the genomic sequence of the mouse homologue have been reported. The full-length human cDNA is 2317 nucleotides in length and contains an open reading frame of 559 amino acids which does not show homology with any reported human genes. The N-terminus contains charged amino acids and a helical loop pattern suggestive of an srp leader sequence for a secreted protein. Fluorescence in situ hybridization using PB39 cDNA as probe mapped the gene to chromosome 11p11.1-p11.2. Comparison of PB39 cDNA sequence with murine sequence available in the public database identifies a region of previously sequenced mouse genomic DNA showing 67% amino acid sequence homology with human PB39. Based on alignment and comparison to the human cDNA the mouse genomic sequence suggests there are at least 14 exons in the mouse gene spread over approximately 100 kb of genomic sequence. Further analysis of PB39 expression in human tissues shows the presence of a unique splice variant mRNA that appears to be primarily associated with fetal tissues and tumors. Interestingly, the unique splice variant appears in prostatic intraepithelial neoplasia, a microscopic precursor lesion of prostate cancer. Comparison of expression levels in normal epithelium and invasive carcinoma, using beta-actin as an internal control, has shown the transcript to be substantially overexpressed in 5 of 10 carcinomas. The current data support the hypothesis that PB39 plays a role in the development of human prostate cancer and will be useful in the analysis of the gene product in further human and murine studies.

The protein similarity information, expression pattern, cellular localization, and map location for the NOV28 protein and nucleic acid disclosed herein suggest that this PB39-like protein may have important structural and/or physiological functions characteristic of the transporters family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV28 nucleic acids and proteins of the invention have applications in the diagnosis and/or treatment of various diseases and disorders. For example, the compositions of the present invention may have efficacy for the treatment of patients suffering from cancer, especially prostate cancer as well as other diseases, disorders and conditions. The expression of PB39 has been shown to be up-regulated in human prostate cancer and the current data support the hypothesis that PB39 plays a role in the development of prostate cancer and will be useful in the analysis of the gene product in further human and murine studies (Genomics Jul. 15, 1998;51(2):282-7).

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV28 protein has multiple hydrophilic regions, each of which can be used as an immunogen. In one embodiment, a contemplated NOV28 epitope is from about amino acids 5 to 7. In another embodiment, a contemplated NOV28 epitope is from about amino acids 70 to 80. In other specific embodiments, contemplated NOV28 epitopes are from about amino acids 200 to 215, 230 to 275, 312 to 310, 350 to 390 and 495 to 510.

NOV29

A disclosed NOV29 (designated CuraGen Acc. No. CG56990-02), which encodes a novel Oxytocin-like protein and includes the 415 nucleotide sequence (SEQ ID NO:85) is shown in Table 29A. An open reading frame for the mature protein was identified beginning with an ATG initiation codon at nucleotides 18-20 and ending with a TGA stop codon at nucleotides 315-317. Putative untranslated regions are underlined in Table 29A, and the start and stop codons are in bold letters.

TABLE 29A

NOV29 Nucleotide Sequence

(SEQ ID NO:85)

CCCAGCGCACCCGCACCATGGCCGGCCCCAGCCTCGCTTGCTGTCTGCTCGGCCTCCTGGCGCTGACCTCCGC
CTGCTACATCCAGAACTGCCCCCTGGGAGGCAAGAGGGCCGCGCCGGAAGAGCTGGGCTGCTTCGTGGGCACC
GCCGAAGCGCTGCGCTGCCAGGAGGAGAACTACCTGCCGTCGCCCTGCCAGTCCGGCCAGAAGGCGTGCGGGA
GCGGGGGCCGCTGCGCGGTCTTGGGCCTCTGCTGCAGCCCGGACGGCTGCCACGCCGACCCTGCCTGCGACGC
GGAAGCCACCTTCTCCCAGCGCTGAAACTTGATGGCTCCGAACACCCTCGAAGCGCGCCACTCGCTTCCCCCA
TAGCCACCCCAGAAATGGTGAAAATAAAATAAAGCAGGTTTTTCTCCTCT

The disclosed NOV29 nucleic acid has been mapped to chromosome 20p13 and has 355 of 407 bases (87%) identical to a gb:GENBANK-ID:HUMOTCBlacc:M25650.1 mRNA from Homo sapiens (Human oxytocin mRNA, complete cds) (E=1.3e⁻⁶¹).

A disclosed NOV29 polypeptide (SEQ ID NO:86) is 99 amino acid residues in length and is presented using the one-letter amino acid code in Table 29B.: The SignalP, Psort and/or Hydropathy results predict that NOV29 has a signal peptide and is likely to be localized to the outside of the cell with a certainty of 0.8200. In alternative embodiments, a NOV29 polypeptide is located to the endoplasmic reticulum (membrane) with a certainty of 0.1000, the endoplasmic reticulum (lumen) with a certainty of 0.1000, or the lysosome (lumen) with a certainty of 0.1000. The SignalP predicts a likely cleavage site for a NOV29 peptide between amino acid positions 19 and 20, i.e. at the sequence TSA-CY.

TABLE 29B

Encoded NOV29 Protein Sequence

(SEQ ID NO:86)

MAGPSLACCLLGLLALTSACYIQNCPLGGKRAAPEELGCFVGTAEALRCQEENYLPSPCQSGQKACGSGGRCAV
LGLCCSPDGCHADPACDAEATFSQR

The NOV29 amino acid sequence was found to have 65 of 65 amino acid residues (100%) identical to, and 65 of 65 amino acid residues (100%) similar to, the 125 amino acid residue ptnr:SWISSNEW-ACC:P01178 protein from Homo sapiens (Human) (OXYTOCIN-NEUROPHYSIN 1 PRECURSOR (OT-NP1) [CONTAINS: OXYTOCIN (OCYTOCIN); NEUROPHYSIN 1]) (E=1.9e⁻¹⁰).

NOV29 is expressed in at least the following tissues: adrenal gland, bone marrow, brain—amygdala, brain—cerebellum, brain—bippocampus, brain—substantia nigra, brain—thalamus, brain-whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus, Hypothalamus, and Whole Organism. Expression information was derived from the tissue sources of the sequences that were included in the derivation of the sequence of NOV29. The sequence is also predicted to be expressed in hypothalamus because of the expression pattern of (GENBANK-ID: gb:GENBANK-ID:HUMOTCBlacc:M25650.1), a closely related Human oxytocin mRNA, complete cds homolog.

NOV29 has homology to the amino acid sequences shown in the BLASTP data listed in Table 29C.

TABLE 29C
BLAST results for NOV29

| Gene Index/ | | Length | Identity | Positives | |
|---|---|---|---|---|---|
| Identifier | Protein/Organism | (aa) | (%) | (%) | Expect |
| gi\|4505537\| | oxytocin- | 125 | 99/125 | 99/125 | 5e−25 |
| ref\|NP_000906.1\| | neurophysin I | | (79%) | (79%) | |
| (NM_000915) | preproprotein; | | | | |
| | oxytocin, prepro- | | | | |
| | (neurophysin I) | | | | |
| | [Homo sapiens] | | | | |
| gi\|386991\| | oxytocin- | 124 | 98/125 | 98/125 | 4e−23 |
| gb\|AAA98806.1\| | neurophysin I | | (78%) | (78%) | |
| (M11186) | [Homo sapiens] | | | | |
| gi\|585553\| | Oxytocin- | 125 | 87/125 | 90/125 | 5e−21 |
| sp\|P01177\|NEU1_PIG | neurophysin 1 | | (69%) | (71%) | |
| | precursor (OT-NPI) | | | | |
| | [Contains: | | | | |
| | Oxytocin | | | | |
| | (Ocytocin); | | | | |
| | Neurophysin 1] | | | | |
| gi\|1346683\| | OXYTOCIN- | 125 | 87/124 | 90/124 | 2e−20 |
| sp\|P13389\|NEU1_SHEEP | NEUROPHYSIN 1 | | (70%) | (72%) | |
| | PRECURSOR (OT-NPI) | | | | |
| | [CONTAINS: | | | | |
| | OXYTOCIN | | | | |
| | (OCYTOCIN); | | | | |
| | NEUROPHYSIN 1] | | | | |
| gi\|128068\| | OXYTOCIN- | 125 | 87/124 | 89/124 | 2e−20 |
| sp\|P01175\|NEU1_BOVIN | NEUROPHYSIN 1 | | (70%) | (71%) | |
| | PRECURSOR (OT-NPI) | | | | |
| | [CONTAINS: | | | | |
| | OXYTOCIN | | | | |
| | (OCYTOCIN); | | | | |
| | NEUROPHYSIN 1] | | | | |

The homology of these sequences is shown graphically in the ClustalW analysis shown in Table 29D.

[image]

[image]

Table 29E lists the domain description from DOhMoN analysis results against NOV29. This indicates that the NOV29 sequence has properties similar to those of other protins known to contain these domains.

TABLE 29E

Domain Analysis of NOV29

gnl|Pfam|pfam00184, hormone5, Neurohypophysial hormones, C-terminal Domain. N-
terminal Domain is in hormone5
CD-Length = 79 residues, 72.2% aligned
Score = 62.4 bits (150), Expect = 1e−11

| | | | | |
|---|---|---|---|---|
| N0V29: | 35 | EELGCFVGTAEALRCQEENYLPSPCQSGQKACGS-GGRCAVLGLCCSPDGCHADPAC | 90 | (SEQ ID NO:375) |
| | | \|\|\|\|\|+\|\|\| \| \|\|\|\|\|\|\|\|\|\|\|\|++\| \| \|\|\| \|\|\|\| \|+\|\| + \| \|\| \| | | |
| Sbjct: | 23 | EELGCYVGTPETARCQEENYLPSPCEAGGKPCGSDAGRCAAPGVCCDSESCVVDPEC | 79 | (SEQ ID NO:376) |

gnl|Smart|smart00003, NH, Neurohypophysial hormones; Vasopressin/oxytocin gene
family.
CD-Length = 79 residues, 72.2% aligned
Score = 60.1 bits (144), Expect = 6e−11

| | | | | |
|---|---|---|---|---|
| NOV29: | 35 | EELGCFVGTAEALRCQEENYLPSPCQSGQKACGS-GGRCAVLGLCCSPDGCHADPAC | 90 | (SEQ ID NO:377) |
| | | \|\|\|\|\|+\|\|\| \| \|\|\|\|\|\|\|\|\|\|\|\|+\|\| + \|\|\| \|\|\|\|\| \|+\|\| + \| \|\|\|+\| | | |
| Sbjct: | 23 | EELGCYVGTPETARCQEENYLPSPCESGGRPCGSDGGRCAAPGICCDSESCAADPSC | 79 | (SEQ ID NO:378) |

Oxytocin (OT), a nonapeptide, was the first hormone to have its biological activities established and chemical structure determined. Oxytocin and vasopressin are structurally and functionally related neurohypophysial peptide hormones. Oxytocin mediates contraction of the smooth muscle of the uterus and mammary gland, while vasopressin has antidiuretic action on the kidney, and mediates vasoconstriction of the peripheral vessels. In common with most active peptides, both hormones are synthesised as larger protein precursors that are enzymatically converted to their mature forms. Members of this family are found in birds, fish, reptiles and amphibians (mesotocin, isotocin, valitocin, glumitocin, aspargtocin, vasotocin, seritocin, asvatocin, phasvatocin), in worms (annetocin), octopi (cephalotocin), locust (locupressin or neuropeptide F1/F2) and in molluscs (conopressins G and S).

It was believed that OT is released from hypothalamic nerve terminals of the posterior hypophysis into the circulation where it stimulates uterine contractions during parturition, and milk ejection during lactation. However, equivalent concentrations of OT were found in the male hypophysis, and similar stimuli of OT release were determined for both sexes, suggesting other physiological functions. Indeed, recent studies indicate that OT is involved in cognition, tolerance, adaptation and complex sexual and maternal behavior, as well as in the regulation of cardiovascular functions. It has long been known that OT induces natriuresis and causes a fall in mean arterial pressure, both after acute and chronic treatment, but the mechanism was not clear. The discovery of the natriuretic family shed new light on this matter. Atrial natriuretic peptide (ANP), a potent natriuretic and vasorelaxant hormone, originally isolated from rat atria, has been found at other sites, including the brain. Blood volume expansion causes ANP release that is believed to be important in the induction of natriuresis and diuresis, which in turn act to reduce the increase in blood volume. Neurohypophysectomy totally abolishes the ANP response to volume expansion. This indicates that one of the major hypophyseal peptides is responsible for ANP release.

The role of ANP in OT-induced natriuresis has been evaluated, and it has been hypothesized that the cardio-renal effects of OT are mediated by the release of ANP from the heart. The presence and synthesis of OT receptors in all heart compartments and the vasculature has been demonstrated. The functionality of these receptors has been established by the ability of OT to induce ANP release from perfused heart or atrial slices. Furthermore, it has been shown that the heart and large vessels like the aorta and vena cava are sites of OT synthesis. Therefore, locally produced OT may have important regulatory functions within the heart and vascular beds. Such functions may include slowing down of the heart or the regulation of local vascular tone.

The protein similarity information, expression pattern, cellular localization, and map location for the NOV29 protein and nucleic acid disclosed herein suggest that this oxytocin-like protein may have important structural and/or physiological functions characteristic of the neurohypophysial hormone family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV29 nucleic acids and proteins of the invention have applications in the diagnosis and/or treatment of various diseases and disorders. For example, the compositions of the present invention may have efficacy for the treatment of patients suffering from reduced muscular tonus of the uterus, lactation problems, cardiovascular conditions, obesity as well as other diseases, disorders and conditions. It has been shown that there is inhibition by elevated circulating OT levels of glucocorticoid-induced, but not basal, leptin secretion in normal weight subjects, suggesting a possible role for OT in the regulatory control of leptin. Furthermore, the results obtained in obese subjects indicate that this regulation is disrupted in obesity (J Clin Endocrinol Metab October 2000;85(10):3683-6). It has also been suggested that OT is involved in cognition, tolerance, adaptation and complex sexual and maternal behavior, as well as in the regulation of cardiovascular functions. Locally produced OT may have important regulatory functions within the heart and vascular beds. Such functions may include slowing down of the heart or the regulation of local vascular tone (Braz J Med Biol Res June 2000;33(6):625-33).

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV29 protein has multiple hydrophilic regions, each of which can be used as an immunogen. In one embodiment, a contemplated NOV29 epitope is from about amino acids 28 to 32. In another embodiment, a contemplated NOV29 epitope is from about amino acids 36 to 37. In other specific embodiments, contemplated NOV29 epitopes are from about amino acids 38 to 39, 46 to 48, 49 to 62 and 88 to 91.

NOV30

One NOVX protein of the invention, referred to herein as NOV30, includes three Thymosin Beta-4-like proteins. The disclosed proteins have been named NOV30a, NOV30b and NOV30c.

NOV30a

A disclosed NOV30a (designated CuraGen Acc. No. CG57330-01), which encodes a novel Thymosin Beta-4-like protein and includes the 201 nucleotide sequence (SEQ ID NO:87) is shown in Table 30A. An open reading frame for the mature protein was identified beginning with an ATG initiation codon at nucleotides 49-51 and ending with a TAA stop codon at nucleotides 199-201. Putative untranslated regions are underlined in Table 30A, and the start and stop codons are in bold letters.

TABLE 30A

NOV30a Nucleotide Sequence

(SEQ ID NO:87)

AGTGGGCATTGCTCAGCTTCCTCTGTGACTACGTCTGACAAGTCCAATATGGATGAGATCGAGAAATTCAGTAAGT
CGAAACTGAAGAAGACAGAAATGCAAGAGAAAAATCCACAGCCTTCCAAGGAATGGATCGAACAGGAGAAGCAAGC
AGGCTTCGTAATGAGGCGTGCATCACCAATATGCACTAAGGGCGAATAA

The disclosed NOV30a nucleic acid sequence maps to chromosome Xq21.3-22 and has 161 of 192 bases (83%) identical to a gb:GENBANK-ID:HUMTHYB41acc:MI7733.1 mRNA from Homo sapiens (Human thymosin beta-4 mRNA, complete cds) (E=1.9e⁻²³).

A disclosed NOV30a polypeptide (SEQ ID NO:88) is 50 amino acid residues in length and is presented using the one-letter amino acid code in Table 30B. The SignalP, Psort and/or Hydropathy results predict that NOV30a does not have a signal peptide and is likely to be localized to the nucleus with a certainty of 0.5800. In alternative embodiments, a NOV30a polypeptide is located to the microbody (peroxisome) with a certainty of 0.3000, the mitochondrial matrix space with a certainty of 0.1000, or the lysosome (lumen) with a certainty of 0.1000.

TABLE 30B

Encoded NOV30a Protein Sequence

(SEQ ID NO:88)

MDEIEKFSKSKLKKTEMQEKNPQPSKEWIEQEKQAGFVMRRASPICTKGE

The NOV30a amino acid sequence was found to have 31 of 36 amino acid residues (86%) identical to, and 31 of 36 amino acid residues (86%) similar to, the 50 amino acid residue ptnr:SWISSPROT-ACC:P20065 protein from Mus musculus (Mouse) (THYMOSIN BETA-4) (E=1.96e⁻¹⁰).

NOV30a is expressed in at least the following tissues: spleen, thymus, lung, and macrophage. Expression information was derived from the tissue sources of the sequences that were included in the derivation of the sequence of NOV30a.

Possible small nucleotide polymorphisms (SNPs) found for NOV30a are listed in Table 30C.

TABLE 30C
SNPs

| Consensus Position | Depth | Base Change | PAF |
|---|---|---|---|
| 16 | 19 | G > T | 0.105 |
| 32 | 19 | C > T | 0.105 |
| 178 | 19 | G > A | 0.105 |

NOV30b

A disclosed NOV30b (designated CuraGen Acc. No. CG57330-03), which encodes a novel Beta Thymosin-like protein and includes the 246 nucleotide sequence (SEQ ID NO:89) is shown in Table 30D. An open reading frame for the mature protein was identified beginning with an ATG initiation codon at nucleotides 31-33 and ending with a TAG stop codon at nucleotides 229-231. Putative untranslated regions are underlined in Table 30b, and the start and stop codons are in bold letters.

TABLE 30D

NOV30b Nucleotide Sequence

(SEQ ID NO:89)

AGTGGGCATTGCTCAGCTTCCTCTGTGACTATGTCTGACAAGTCCAATATGGATGAGATCGAGAAATTCAGTAAG
TCGAAACTGAAGAAGACAGAAATGCAAGAGAAAAATCCACAGCCTTCCAAGGAATGGATCGAACAGGAGAAGCAA
GCAGGCTTCGTAATGAGGCGTGCATCGCCAATATGCACTGTTCATTCCACAAAGCATTGCTTTCTATTTTACTTC
TTTTAGCTGTTTAACTTTGAA

The disclosed NOV30b nucleic acid sequence maps to chromosome 8 and has 216 of 249 bases (86%) identical to a gb:GENBANK-ID:HUMTHYB41acc:MI7733.1 mRNA from Homo sapiens (Human thymosin beta-4 mRNA, complete cds) (E=1. Ie⁻³⁴).

A disclosed NOV30b polypeptide (SEQ ID NO:90) is 66 amino acid residues in length and is presented using the one-letter amino acid code in Table 30E. The SignalP, Psort and/or Hydropathy results predict that NOV30b does not have a signal peptide and is likely to be localized to the microbody (peroxisome) with a certainty of 0.7095. In alternative embodiments, a NOV30b polypeptide is located to the mitochondrial matrix space with a certainty of 0.1000 or the lysosome (lumen) with a certainty of 0.1000.

TABLE 30E

Encoded NOV30b Protein Sequence

| | |
|---|---|
| MSDKSNMDEIEKFSKSKLKKTEMQEKNPQPSKEWIEQEKQAGFVMRRASPICTVHSTKHCFLFYFF | (SEQ ID NO:90) |

The NOV30b amino acid sequence was found to have 36 of 42 amino acid residues (85%) identical to, and 37 of 42 amino acid residues (88%) similar to, the 44 amino acid residue ptnr:SPTREMBL-ACC:Q9NQQ5 protein from Homo sapiens (Human) (DJ1071L10.1 (THYMOSIN/INTERFERON—INDUCIBLE MULTIGENE FAMILY)) (E=5.0e⁻¹³).

Expression information was derived from the tissue sources of the sequences that were included in the derivation of the sequence of NOV30b The sequence is predicted to be expressed in the following tissues because of the expression pattern of (GENBANK-ID: gb:GENBANK-ID:HUMTHYB41acc:M 17733.1), a closely related Human thymosin beta-4 mRNA, complete cds homolog in species Homo sapiens: Lung, small cell carcinoma.

NOV30c

A disclosed NOV30c (designated CuraGen Acc. No. CG57330-02), which encodes a novel Thymosin Beta-4-like protein and includes the 201 nucleotide sequence (SEQ ID NO:91) is shown in Table 30F. An open reading frame for the mature protein was identified beginning with an ATG initiation codon at nucleotides 31-33 and ending with a TAA stop codon at nucleotides 199-201. Putative untranslated regions are underlined in Table 30A, and the start and stop codons are in bold letters.

TABLE 30F

NOV30c Nucleotide Sequence

(SEQ ID NO:91)

AGTGGGCATTGCTCAGCTTCCTCTGTGACTATGTCTGACAAGTCCAATATGGATGAGATCGAGAAATTCAGTAAG
TCGAAACTGAAGAAGACAGAAATGCAAGAGAAAAATCCACAGCCTTCCAAGGAATGGATCGAACAGGAGAAGCAA
GCAGGCTTCGTAATGAGGCGTGCATCACCAATATGCACTAAGGGCGAATAA

The disclosed NOV30c nucleic acid sequence maps to chromosome X and has 162 of 192 bases (84%) identical to a gb:GENBANK-ID:HUMTHYB41acc:M17733.1 mRNA from Homo sapiens (Human thymosin beta-4 mRNA, complete cds) (E=7.5e⁻²⁴).

The NOV30c polypeptide (SEQ ID NO:92) is 56 amino acid residues in length and is presented using the one-letter amino acid code in Table 30G. The SignalP, Psort and/or Hydropathy results predict that NOV30c does not have a signal peptide and is likely to be localized to the nucleus with a certainty of 0.5600. In alternative embodiments, a NOV30c polypeptide is located to the microbody (peroxisome) with a certainty of 0.3000, the mitochondrial matrix space with a certainty of 0.1000, or the lysosome (lumen) with a certainty of 0.1000.

TABLE 30G
Encoded NOV3Oc Protein Sequence

| | |
|---|---|
| MSDKSNMDEIEKFSKSKLKKTEMQEKNPQPSKEWIEQEKQAGFVNRRASPICTKGE | (SEQ ID NO:92) |

The NOV30c amino acid sequence was found to have 36 of 42 amino acid residues (85%) identical to, and 37 of 42 amino acid residues (88%) similar to, the 44 amino acid residue ptnr:SPTREMBL-ACC:Q9NQQ5 protein from Homo sapiens (Human) (DJ1071L10.1 (THYMOSIN/INTERFERON—INDUCIBLE MULTIGENE FAMILY)) (E=4.5e⁻¹³).

NOV30c is expressed in at least the following tissues: adrenal gland, bone marrow, brain—amygdala, brain—cerebellum, brain—hippocampus, brain—substantia nigra, brain—thalamus, brain-whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the sequence of NOV30c.

Possible small nucleotide polymorphisms (SNPs) found for NOV30c are listed in Tables 30H and 30I.

TABLE 30H
SNPs

| Consensus Position | Depth | Base Change | PAF |
|---|---|---|---|
| 16 | 47 | G > T | 0.043 |
| 32 | 47 | T > C | 0.468 |
| 183 | 23 | G > A | 0.087 |

TABLE 30I
SNPs

| | Nucleotide | | Amino Acid | |
|---|---|---|---|---|
| Variant | Position | Base Change | Position | Base Change |
| 13377029 | 89 | A > G | 14 | Lys > Arg |
| 13377030 | 148 | C > T | 148 | Gln > End |
| 13377031 | 150 | A > G | 150 | NA |

Homologies to any of the above NOV30a, NOV30b and NOV30c proteins will be shared by the other NOV30 proteins insofar as they are homologous to each other as shown above. Any reference to NOV30 is assumed to refer to NOV30a, NOV30b and NOV30c proteins in general, unless otherwise noted.

NOV30a, NOV30b and NOV30c are very closely homologous as is shown in the amino acid alignment in Table 30J

[image]

NOV30 also has homology to the amino acid sequences shown in the BLASTP data listed in Table 30K

TABLE 30K
BLAST results for NOV30a

| Gene Index/ | | Length | Identity | Positives | |
|---|---|---|---|---|---|
| Identifier | Protein/Organism | (aa) | (%) | (%) | Expect |
| gi\|17451239\| | similar to | 158 | 37/37 | 37/37 | 1e−12 |
| ref\|XP_070564.1\| | ribosomal protein | | (100%) | (100%) | |
| (XM_070564) | L10 (H. sapiens) | | | | |
| | [Homo sapiens] | | | | |
| gi\|2143995\| | thymosin beta-4 | 56 | 31/36 | 31/36 | 0.015 |
| pir\|\|I52084 | precursor - rat | | (86%) | (86%) | |
| | (fragment) | | | | |
| gi\|136580\| | Thymosin beta-4 (T | 50 | 31/36 | 31/36 | 0.089 |
| sp\|P20065\|TYB4_MOUSE | beta 4) | | (86%) | (86%) | |
| gi\|464974\| | Thymosin beta-4 (T | 43 | 31/36 | 31/36 | 0.089 |
| sp\|P34032\|TYB4_RABIT | beta 4) | | (86%) | (86%) | |
| gi\|10946578\| | thymosin, beta 4, X | 44 | 31/36 | 31/36 | 0.089 |
| ref\|NP_067253.1\| | chromosome; | | (86%) | (86%) | |
| (NM_021278) | prothymosin beta 4 | | | | |
| | [Mus musculus] | | | | |

The homology of these sequences is shown graphically in the ClustalW analysis shown in Table 30L.

[image]

Tables 30M and 30N list the domain description from DOMAIN analysis results against NOV30. This indicates that the NOV30 sequence has properties similar to those of other proteins known to contain these domains.

TABLE 30M
Domain Analysis of NOV30

gnl |Smart|smart00152, THY, Thymosin beta actin-binding motif.
CD-Length = 37 residues, 97.3% aligned
Score = 32.0 bits (71), Expect = 0.009

| | | | | |
|---|---|---|---|---|
| NOV30: | 1 | MDEIEKFSKSKLKKTEMQEKNPQPSKEWIEQEKQAG | 36 | (SEQ ID NO:384) |
| | | \|\|\|\| \| \|\|\|\|\| \|\|\| \|\|\|\| \|\|\|\|\|\| | | |
| Sbjct: | 1 | TDEIENFDSENLKKTETIEKNVLPSKEDIEQEKQLQ | 36 | (SEQ ID NO:385) |

TABLE 30N
Domain Analysis of NOV30

hmmpfam—search a single seq against HMM database
HSIM file: pfamHMNs
Scores for sequence family classification (score includes all
domains)

| | | | | |
|---|---|---|---|---|
| Model | Description Score | E-value | N | |
| Thymosin | Thymosin beta-4 family | 57.1 | 3.7e−13 | 1 |
| (INTERPRO) | | | | |

Parsed for domains:

| | | | | | | | |
|---|---|---|---|---|---|---|---|
| Model | Domain | seq-f | seq-t | hmm-f | hmm-t | score | E-value |
| Thymosin | 1/1 | 1 36 [. | 1 | 41 [] | 57.1 | 3.7e−13 | |

Alignments of top-scoring domains:
Thymosin: domain 1 of 1, from 1 to 36: score 57.1, E =3.7e−13

| | | |
|---|---|---|
| | *->sDKPdleEiasFDKaKLKKtEtqEKnpLPtKEtiEqEKqae<* | (SEQ ID NO: 386) |
| | ++\|\|++\| \|+\|\|\|\|\|\| \|\|\|\|\|+\|+\|\|+\|\|\|\|\|\|\|+ | |
| NOV30a | 1 -----MDEIEKFSKSKLKKTEMQEKNPQPSKEWIEQEKQAG 36 | (SEQ ID NO:387) |

Thymosin beta-4 is a small polypeptide whose exact physiological role is not yet known. It was first isolated as a thymic hormone that induces terminal deoxynucleotidyl-transferase. It is found in high quantity in thymus and spleen but is widely distributed in many tissues. It has also been shown to bind to actin monomers and thus to inhibit actin polymerization. See Interpro IPR001152:

A number of peptides closely related to thymosin beta-4 belong to this family. They include, thymosin beta-9 (and beta-8) in bovine and pig, thymosin beta-10 in man and rat, thymosin beta-11 and beta-12 in trout and human Nb thymosin beta. Thymosin was originally isolated from a partially purified extract of calf thymus, thymosin fraction 5, which induced differentiation of T cells and was partially effective in some immunocompromised animals. Further studies demonstrated that the molecule is ubiquitous; it had been found in all tissues and cell lines analyzed. It is found in highest concentrations in spleen, thymus, lung, and peritoneal macrophages. Thymosin-beta-4 (T-beta-4) is an actin monomer sequestering protein that may have a critical role in modulating the dynamics of actin polymerization and depolymerization in nonmuscle cells. Its regulatory role is consistent with the many examples of transcriptional regulation of T-beta-4 and of tissue-specific expression. Lymphocytes have a unique T-beta-4 transcript relative to the ubiquitous transcript found in many other tissues and cells. Rat thymosin-beta-4 is synthesized as a 44-amino acid propeptide which is processed into a 43-amino acid peptide by removal of the first methionyl residue. The molecule does not have a signal peptide. Human thymosin-beta-4 has a high degree of homology to rat thymosin-beta-4; the coding regions differ by only 9 nucleotides, and these are all silent base changes.

A cDNA encoding thymosin-beta-4 has been isolated by differential screening of a cDNA library prepared from leukocytes of an acute lymphocytic leukemia patient. Using Northern blot analysis, the expression of the thymosin-beta-4 mRNA in various primary myeloid and lymphoid malignant cell lines and in hemopoietic cell lines was studied. The pattern of thymosin-beta-4 gene expression suggests that it may be involved in an early phase of the host defense mechanism. A cDNA clone for the human interferon-inducible gene 6-26 has been isolated and shown to be identical to that for the human thymosin-beta-4 gene. By use of a panel of human rodent somatic cell hybrids, it has been shown that the cDNA recognized 7 genes, members of a multigene family, present on chromosomes 1, 2, 4, 9, 11, 20, and X. These genes are symbolized TMSL1, TMSL2, etc., respectively.

In the mouse there is a single Tmsb4 gene and the lymphoid-specific transcript is generated by extending the ubiquitous exon 1 with an alternate downstream splice site. By interspecific backcross mapping, the mouse gene (designated Ptmb4) has been located to the distal region of the mouse X chromosome, linked to Btk and Gja6. Thus, the human gene could be predicted to reside on the X chromosome in the general region of Xq21.3-q22, where BTK is located. By analysis of somatic cell hybrids, the thymosin-beta-4, or TB4X, gene was mapped to the X chromosome. A homologous gene, TB4Y, is present on the Y chromosome. The TB4X gene escapes X inactivation, and it has been suggested that it should be investigated as a candidate gene for Turner syndrome. Thymosin-beta-4 induces the expression of terminal deoxynucleotidyl transferase activity in vivo and in vitro, inhibits the migration of macrophages, and stimulates the secretion of hypothalamic luteinizing hormone-releasing hormone. It has also been suggested that thymosin beta-4 is required for the metastasis of melanoma cells.

The protein similarity information, expression pattern, cellular localization, and map location for the NOV30 protein and nucleic acid disclosed herein suggest that this thymosin beta-4-like protein may have important structural and/or physiological functions characteristic of the thymosin beta-4 family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV30 nucleic acids and proteins of the invention have applications in the diagnosis and/or treatment of various diseases and disorders. For example, the compositions of the present invention may have efficacy for the treatment of patients suffering from agammaglobulinemia, type 1, X-linked; agammaglobulinemia, X-linked; XLA and isolated growth hormone deficiency; premature ovarian failure; idiopathic thrombocytopenic purpura, immunodeficiencies, graft versus host disease; systemic lupus erythematosus, autoimmune disease, asthma, emphysema, scleroderma, ARDS; allergies, cancer, compromised immune system as well as other diseases, disorders and conditions.

These antibodies may be generated according to methods known in the art, using prediction from hydrophobicity charts, as described in the “Anti-NOVX Antibodies” section below. The disclosed NOV30 protein has multiple hydrophilic regions, each of which can be used as an immunogen. In one embodiment, a contemplated NOV30 epitope is from about amino acids 11 to 13. In another embodiment, a contemplated NOV30 epitope is from about amino acids 14 to 16. In other specific embodiments, contemplated NOV30 epitopes are from about amino acids 17 to 19, 21 to 25, 26 to 27, 31 to 32, 35 to 36 and 37 to 41.

NOV31

One NOVX protein of the invention, referred to herein as NOV31, includes two Myelin P2-like nucleic acids encoding the same protein. The disclosed nucleic acids have been named NOV31a and NOV31b.

NOV31a

A disclosed NOV31a (designated CuraGen Acc. No. CG57344-01), which encodes a novel Myelin P2-like protein and includes the 457 nucleotide sequence (SEQ ID NO:93) is shown in Table 31A. An open reading frame for the mature protein was identified beginning with an ATG initiation codon at nucleotides 21-23 and ending with a TAA stop codon at nucleotides 441-443. Putative untranslated regions are underlined in Table 31A, and the start and stop codons are in bold letters.

TABLE 31A
NOV31a Nucleotide Sequence

(SEQ ID NO:93)

ATCAACTTATCTCAGACAGAATGATTGACCAGCTCCAAGGAACATGGAAGTCCATTTCTTGTGAAAATTCCGAAGACT
ACATGAAGGAGCTGGGTATAGGAAGAGCCAGCAGGAAACTGGGCCGTTTGGCAAAACCCACTGTGACCATCAGTACAG
ATGGAGATGTCATCACAATAAAAACCAAAAGCATCTTTAAAAATAATGAGATCTCCTTTAAGCTGGGAGAAGAGTTTG
AGGAAATCACGCCAGGTGGCCACAAAACAAAGAGTAAAGTAACCTTAGATAAGGAGTCCCTGATTCAAGTTCAGGACT
GGGATGGCAAAGAAACCACCATAACGAGAAAGCTGGTGGATGGGAAAATGGTGGTGGAAAGTACTGTGAACAGTGTTA
TCTGTACACGAACATACGAGAAAGTATCATCAAACTCAGTCTCAAACTCTTAAGGCTTTCTCAAGCT

The disclosed NOV31a nucleic acid sequence maps to chromosome 8 and has 298 of 418 bases (71%) identical to a gb:GENBANK-ID:RABPLP2|acc:J03744.1 mRNA from Oryctolagus cuniculus (Rabbit myelin P2 mRNA, complete cds) (E=3.9e⁻³⁸).

NOV31b

A disclosed NOV31b (designated CuraGen Acc. No. CG57344-02), also encodes a novel Myelin P2-like protein. This nucleic acid includes a 426 nucleotide sequence which differs from NOV31a by having a 20 nucleotide deletion at the 5′ end (the 5′UTR), an 11 nucleotide deletion at the 3′ end and one mutation (T>C) at position 251 (numbered relative to NOV31a). An open reading frame for the mature protein was identified beginning with an ATG initiation codon at nucleotides 1-3 and ending with a TAA stop codon at nucleotides 421-423. Putative untranslated regions are underlined in Table 31b, and the start and stop codons are in bold letters.

The disclosed NOV31b nucleic acid sequence maps to chromosome 8 and has 291 of 403 bases (72%) identical to a gb:GENBANK-ID:RABPLP2|acc:J03744.1 mRNA from Oryctolagus cuniculus (Rabbit myelin P2 mRNA, complete cds) (E=5.8e⁻³⁸).

The NOV31 polypeptide (SEQ ID NO:94) is 140 amino acid residues in length and is presented using the one-letter amino acid code in Table 31B. The SignalP, Psort and/or Hydropathy results predict that NOV31a does not have a signal peptide and is likely to be localized to the cytoplasm with a certainty of 0.6500. In alternative embodiments, a NOV31a polypeptide is located to the mitochondrial matrix space with a certainty of 0.1000 or the lysosome (lumen) with a certainty of 0.1000.

TABLE 31B
Encoded NOV31 Protein Sequence

(SEQ ID NO:94)

MIDQLQGTWKSISCENSEDYMKELGIGRASRKLGRLAXPTVTISTDGDVITIKTKSIFKNNEISFKLGEEFEEIT
PGGHKTKSKVTLDKESLIQVQDWDGKETTITRKLVDGKMVVESTVNSVICTRTYEKVSSNSVSNS

The NOV31 amino acid sequence was found to have 86 of 132 amino acid residues (65%) identical to, and 102 of 132 amino acid residues (77%) similar to, the 132 amino acid residue ptnr:pir-id:MPRB2 protein from rabbit (myelin P2 protein) (E=1.7e⁻⁴¹).

NOV31 is expressed in at least the following tissues because of the expression pattern of (GENBANK-ID: gb:GENBANK-ID:RABPLP2|acc:J03744.1) a closely related Rabbit myelin P2 mRNA, complete cds homolog in species Oryctolagus cuniculus:sciatic nerve, spinal cord, and brain.

Possible small nucleotide polymorphisms (SNPs) found for NOV31 are listed in Table 31C.

TABLE 31C
SNPs

| Consensus Position | Depth | Base Change | PAF |
|---|---|---|---|
| 196 | 21 | A>G | 0.095 |

Homologies to any of the above NOV31 proteins will be shared by the other NOV31 proteins insofar as they are homologous to each other as shown above. Any reference to NOV31 is assumed to refer to NOV31a and NOV31b proteins in general, unless otherwise noted.

NOV31 also has homology to the amino acid sequences shown in the BLASTP data listed in Table 31D.

TABLE 31D
BLAST results for NOV31a

| Gene Index/ | | Length | Identity | Positives | |
|---|---|---|---|---|---|
| Identifier | Protein/Organism | (aa) | (%) | (%) | Expect |
| gi\|12838509\|dbj\| | data source: SPTR, | 132 | 106/132 | 119/132 | 3e−52 |
| BAB24227.1\| | source key: P24526, | | (80%) | (89%) | |
| (AK005765) | evidence: ISS˜putat | | | | |
| | ive˜similar to | | | | |
| | MYELIN P2 PROTEIN | | | | |
| | [Mus musculus] | | | | |
| gi\|127727\|sp\|P02 | Myelin P2 protein | 132 | 86/132 | 102/132 | 1e−38 |
| 691\|MYP2_RABIT | | | (65%) | (77%) | |
| gi\|4505909\|ref\|N | peripheral myelin | 132 | 87/132 | 101/132 | 3e−38 |
| P_002668.1\| | protein 2; M-FABP | | (65%) | (75%) | |
| (NM_002677) | [Homo sapiens] | | | | |
| gi\|127726\|sp\|P24 | Myelin P2 protein | 132 | 82/132 | 99/132 | 6e−38 |
| 526\|MYP2_MOUSE | | | (62%) | (74%) | |
| gi\|1353194\|sp\|P4 | Fatty acid-binding | 132 | 78/131 | 100/131 | 2e−37 |
| 8035\|FABA_BOVIN | protein, adipocyte | | (59%) | (75%) | |
| | (AFABP) (Adipocyte | | | | |
| | lipid-binding | | | | |
| | protein) (ALBP) | | | | |

The homology of these sequences is shown graphically in the ClustalW analysis shown in Table 31E.

[image]

Table 31F lists the domain description from DOMAIN analysis results against NOV31. This indicates that the NOV31 sequence has properties similar to those of other proteins known to contain these domains.

TABLE 31F
Domain Analysis of NOV31

gnl|Pfam|pfam0061, lipocalin, Lipocalin/cytosolic fatty-acid binding
protein family. Lipocalins are transporters for small hydrophobic
molecules, such as lipids, steroid hormones, bilins, and retinoids.
Alignment subsumes both the lipocalin and fatty acid binding protein
signatures from PROSITE. This is supported on structural and functional
grounds. Structure is an eight-stranded beta barrel.
CD-Length = 145 residues, 100.0% aligned
Score = 56.6 bits (135), Expect = 9e−10

| | | | | |
|---|---|---|---|---|
| NOV31: | 4 | QLQGTWKSISCENSEDYMK-ELGIGRASRELGRLAXPTVTISTDGDVITIKTKSIFKNN | 61 | (SEQ ID NO:393) |
| | | + \| \| 30 + \|++ +\| \|\|\|+ =51 +\|\| \| + \| =51 \|\| \| + \| | | |
| Sbjct: | 1 | KFAGKWYLVASANFDPELKEELGVLEATRKEITPLKEGNLEIVFDGDKNGI-CEETFGKL | 59 | (SEQ ID NO:394) |
| NOV31: | 62 | EISFKLGEEFEEITPGGHKTKSKTLDEESLIQVQDWDGKETTITRKLVDGKMVVESTV- | 120 | |
| | | \| + \|\|\| \|\|+ \| \| =51 + \|\| \|\| \|\|+ \| +\| + | | |
| Sbjct: | 60 | EKTKKLGVEFDYYTGDNRFVVLDTDYDNYLLVCVQKGDGNETSRTAELYGRTPELSPEAL | 119 | |
| NOV31: | 121 | -------------NSVICTRTYEKV | 132 | |
| | | ++\|+\|\|\| \|+ | | |
| Sbjct: | 120 | ELFETATKELGIPEDNVVCTRQTERC | 145 | |

See InterPro IPR000463: Cytosolic fatty-acid binding protein. The Fatty Acid-Binding Proteins (FABPs) are a family of proteins that are principally located in the cytosol and are characterized by the ability to bind to hydrophobic ligands, such as fatty acids, retinol, retinoic acid, bile salts and pigments. Recently, a number of family members have been identified that are secreted, such as gastrotropin and mammary-derived growth inhibitor. The family is implicated in general lipid metabolism, acting as intracellular transporters of hydrophobic metabolic intermediates and as carriers of lipids between membranes. The FABPs exhibit a high degree both of sequence and structural similarity. They are small, 12-18 kDa, soluble proteins composed of 110-160 residues. Their crystal structures show them to be 10-stranded anti-parallel beta-barrels with a +1, +1 topology, which wrap around an internal cavity to form a ligand binding site. The anti-parallel beta-barrel fold is also exploited by the lipocalins, which function similarly by binding small hydrophobic molecules. Similarity at the sequence level, however, is less obvious, being confined to a single short N-terminal motif. Proteins which transport small hydrophobic molecules such as steroids, bilins, retinoids, and lipids share limited regions of sequence homology and a common tertiary structure architecture. This is an eight stranded antiparallel beta-barrel with a repeated +1 topology enclosing a internal ligand binding site. The name ‘lipocalin’ has been proposed for this protein family, but cytosolic fatty-acid binding proteins are also included. The sequences of most members of the family, the core or kernal lipocalins, are characterized by three short conserved stretches of residues, while others, the outlier lipocalin group, share only one or two of these.

Myelin is a multilamellar compacted membrane structure that surrounds and insulates axons, facilitating the conduction of nerve impulses. It is composed predominantly of lipids, with proteins accounting for about 30% of its net weight. Schwann cells are responsible for myelin formation in the peripheral nervous system. Peripheral myelin protein-2 (PMP2), a small basic protein, is one of the major proteins of peripheral myelin and appears to be related to the transport of fatty acids or the metabolism of myelin lipids. Hayasaka et al. (1991) noted that PMP2 (which they also called myelin P2 protein, MP2) was shown to have lipid-binding activity. Thus, MP2 protein may have an important role in the organization of compact myelin.

Hayasaka et al. (1991) isolated a full-length cDNA of MP2 protein of peripheral myelin from a cDNA library of human fetus spinal cord. It was found to contain a 393-bp open reading frame encoding a polypeptide of 131 residues. The deduced amino acid sequence is highly homologous to myelin P2 protein from other species. Hayasaka et al. (1993) cloned the genomic PMP2 sequence, which is about 8 kb long and consists of 4 exons. By spot-blot hybridization (FISH) of flow-sorted human chromosomes and fluorescence in situ hybridization, Hayasaka et al. (1993) mapped the PMP2 gene to chromosome 8q21.3-q22.1. This is the same region as that in which the autosomal recessive form of Charcot-Marie-Tooth peroneal muscular atrophy (CMT4A) has been mapped. Thus, the PMP2 gene was a prime candidate for the site of the mutation in that disorder. Narayanan et al. (1994) reported the partial structure of the PMP2 gene. Using a panel of human/hamster somatic cell hybrids and by FISH, they localized the gene to 8q21. Ben Othmane et al. (1995) created a 7-Mb YAC contig spanning the region of 8q 13-q21 to which the CMT4A gene was mapped. This contig was used to map 9 additional microsatellites and 6 STSs to this region; subsequent haplotype analysis narrowed the CMT4A flanking interval to less than 1 cM. Using SSCP and the physical map, they could demonstrate that the PMP2 gene is not the defect in CMT4A.

Myelin P2 is a 14,800-Da cytosolic protein found in rabbit sciatic nerves. It belongs to a family of fatty acid binding proteins and shows a 72% amino acid sequence similarity to aP2/422, the adipocyte lipid binding protein, a 58% sequence similarity to rat heart fatty acid binding protein, and a 40% sequence similarity to cellular retinoic acid binding protein. In order to isolate cDNA clones representing P2, a cDNA library was constructed from poly(A+) RNA isolated from sciatic nerves of 10-day-old rabbit pups. By use of a mixed synthetic oligonucleotide probe based on the rabbit P2 amino sequence, 12 cDNA clones were selected from about 25,000 recombinants. Four of these were further characterized. They contained an open reading frame, which when translated, agreed at 128 out of 131 residues with the known rabbit P2 amino acid sequence. These cDNAs recognize a 1.9-kilobase mRNA present in sciatic nerve, spinal cord, and brain, but not present in liver or heart. The levels of P2 mRNA parallel myelin formation in sciatic nerve and spinal cord with maximal amounts being detected at about 15 postnatal days. P2 protein is a small basic protein (Mr=14,820) found in peripheral nerve myelin and spinal cord myelin. There is now overwhelming evidence that P2 protein is the crucial antigen involved in the induction of experimental allergic neuritis, an autoimmune disease of the peripheral nervous system. The complete amino acid sequence of rabbit P2 protein was derived by sequence analysis of cyanogen bromide peptides and peptides obtained by proteolysis using Staphylococcus aureus V8 enzyme, trypsin, or clostripain. There are 131 amino acids and an excess of the basic amino acids lysine and arginine; histidine is absent. There are 3 highly hydrophobic regions in the P2 molecule. Probability analysis of the sequence predicts a high degree of beta structure, essentially in agreement with CD data.

The protein similarity information, expression pattern, cellular localization, and map location for the NOV31 protein and nucleic acid disclosed herein suggest that this Myelin P2-like protein may have important structural and/or physiological functions characteristic of the Fatty Acid Binding Protein family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV31 nucleic acids and proteins of the invention have applications in the diagnosis and/or treatment of various diseases and disorders. For example, the compositions of the present invention will have efficacy for the treatment of patients suffering from: Charcot-Marie-Tooth peroneal muscular atrophy, allergic neuritis (an autoimmune disease of the peripheral nervous system), Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection as well as other diseases, disorders and conditions.

These antibodies may be generated according to methods known in the art, using prediction from hydrophobicity charts, as described in the “Anti-NOVX Antibodies” section below. The disclosed NOV31 protein has multiple hydrophilic regions, each of which can be used as an immunogen. In one embodiment, a contemplated NOV31 epitope is from about amino acids 10 to 12. In another embodiment, a contemplated NOV31 epitope is from about amino acids 20 to 21. In other specific embodiments, contemplated NOV31 epitopes are from about amino acids 22 to 25, 30 to 31, 38 to 42, 50 to 51, 58 to 60, 65 to 67, 70 to 73, 75 to 78, 81 to 83, 84 to 85, 86 to 87, 90 to 100, 105 to 110, 110-112, 121 to 123 and 130 to 133.

NOV32

One NOVX protein of the invention, referred to herein as NOV32, includes two Testis Lipid-Binding Protein-like proteins. The disclosed proteins have been named NOV32a and NOV32b.

NOV32a

A disclosed NOV32a (designated CuraGen Acc. No. CG57346-01), which encodes a novel Testis Lipid-Binding Protein-like protein and includes the 408 nucleotide sequence (SEQ ID NO:95) is shown in Table 32A. An open reading frame for the mature protein was identified beginning with an ATG initiation codon at nucleotides 10-12 and ending with a TGA stop codon at nucleotides 400-402. Putative untranslated regions are underlined in Table 32A, and the start and stop codons are in bold letters.

TABLE 32A
NOV32a Nucleotide Sequence

(SEQ ID NO:95)

TGTTCCATGATGGTTGAGCCCTTCTTGGGAACCTGGAAGCTGGTCTCCAGTGAAAACTTTGAGGATTACATGAAAG
AACTGGGTTTCGCAGCCCGGAACATGGCAGGGTTAGTGAAACCGACAGTAACTATTAGTGTTGATGGGAAAATGAT
GACCATAAGAACAGAAAGTTCTTTCCAGGACACTAAGATCTCCTTCAAGCTGGGGGAAGAATTTGATGAAACTACA
GCAGACAACCGGAAAGTAAAGAGCACCATAACATTAGAGAATGGCTCAATGATTCACGTCCAAAAATGGCTTGGCA
AAGAGACAACAATCAAAAGAAAAATTGTGGATGAAAAAATGGTAGTGGAATGTAAAATGAATAATATTGTCAGCAC
CAGAATCTACGAAAAGGTGTGAAGAAAG

The disclosed NOV32a nucleic acid sequence maps to chromosome 8 and has 321 of 413 bases (77%) identical to a gb:GENBANKID:RRU07870|acc:UO7870.1 mRNA from Rattus norvegicus (Rattus norvegicus testis lipid binding protein mRNA, complete cds) (E=9.4e⁻⁴⁷).

A disclosed NOV32a polypeptide (SEQ ID NO:96) is 130 amino acid residues in length and is presented using the one-letter amino acid code in Table 32B. The SignalP, Psort and/or Hydropathy results predict that NOV32a does not have a signal peptide and is likely to be localized to the cytoplasm with a certainty of 0.4500. In alternative embodiments, a NOV32a polypeptide is located to the mitochondrial matrix space with a certainty of 0.1000, the lysosome (lumen) with a certainty of 0.1000 or the microbody (peroxisome) with a certainty of 0.1000.

TABLE 32B
Encoded NOV32a Protein Sequence

(SEQ ID NO: 96)

MVEPFLGTWKLVSSENFEDYMKELGFAARNMAGLVKPTVTISVDGKMMTIRTESSFQDTKISFKLGEEFDETTAD
NRKVKSTITLENGSMIHVQKWLGKETTIKRKIVDEKMVVECKMNNIVSTRIYEKV

The NOV32a amino acid sequence was found to have 90 of 132 amino acid residues (68%) identical to, and 112 of 132 amino acid residues (84%) similar to, the 132 amino acid residue ptnr:SWISSPROT-ACC:008716 protein from Mus musculus (Mouse) (TESTIS LIPID BINDING PROTEIN (TLBP) (15 KDA PERFORATORIAL PROTEIN) (PERF 15)) (E=3.1e⁻⁴⁴).

NOV32a is predicted to be expressed in testis because of the expression pattern of (GENBANK-ID: gb:GENBANK-ID:RRU07870|acc:U07870.1), a closely related Rattus norvegicus testis lipid binding protein mRNA, complete cds homolog in species Rattus norvegicus.

NOV32b

A disclosed NOV32b (designated CuraGen Acc. No. CG57346-02), which encodes a novel Testis Lipid Binding Protein-like protein and includes the 459 nucleotide sequence (SEQ ID NO:97) is shown in Table 32C. An open reading frame for the mature protein was identified beginning with an ATG initiation codon at nucleotides 28-30 and ending with a TGA stop codon at nucleotides 427-429. Putative untranslated regions are underlined in Table 32b, and the start and stop codons are in bold letters.

TABLE 32C
NOV32b Nucleotide Sequence

(SEQ ID NO:97)

CGAGTGGCTCTTCTCAGCAAGTGTTCCATGATGGTTGAGCCCTTCTTGGGAACCTGGAAGCTGGTCTCCAGTGAA
AACTTTGAGGATTACATGAAAGAACTGGGTGTGAATTTCGCAGCCCGGAACATGGCAGGGTTAGTGAAACCGACA
GTAACTATTAGTGTTGATGGGAAAATGATGACCATAAGAACAGAAAGTTCTTTCCAGGACACTAAGATCTCCTTC
AAGCTGGGGGAAGAATTTGATGAAACTACAGCAGACAACCGGAAAGTAAAGAGCACCATAACATTAGAGAATGGC
TCAATGATTCACGTCCAAAAATGGCTTGGCAAAGAGACAACAATCAAAAGAAAAATTGTGGATGAAAAAATGGTA
GTGGAATGTAAAATGAATAATATTGTCAGCACCAGAATCTACGAAAAGGTGTGAAGAAAGGTCCACAGCAATGAA
AACTTGTTC

The disclosed NOV32b nucleic acid sequence maps to chromosome 8 and has 347 of 446 bases (77%) identical to a gb:GENBANK-ID:RRU07870|acc:U07870.1 mRNA from Rattus norvegicus (Rattus norvegicus testis lipid binding protein mRNA, complete cds) (E=3.5e⁻⁵²).

The NOV32b polypeptide (SEQ ID NO:98) is 133 amino acid residues in length and is presented using the one-letter amino acid code in Table 32D. The SignalP, Psort and/or Hydropathy results predict that NOV32b does not have a signal peptide and is likely to be localized to the cytoplasm with a certainty of 0.6500. In alternative embodiments, a NOV32b polypeptide is located to the mitochondrial matrix space with a certainty of 0.1000, the lysosome (lumen) with a certainty of 0.1000 or the microbody (peroxisome) with a certainty of 0.0138.

TABLE 32D
Encoded NOV32b Protein Sequence

(SEQ ID NO:98)

MMVEPFLGTWKLVSSENFEDYMKELGVNFAARNMAGLVKPTVTISVDGKMMTIRTESSFQDTKISFKLGEEFD
ETTADNRKVKSTITLENGSMIHVQKWLGKETTIKRKIVDEKMVVECKMNNIVSTRIYEKV

The NOV32b amino acid sequence was found to have 91 of 132 amino acid residues (68%) identical to, and 113 of 132 amino acid residues (85%) similar to, the 132 amino acid residue ptnr:SWISSPROT-ACC:008716 protein from Mus musculus (Mouse) (TESTIS LIPID BINDING PROTEIN (TLBP) (15 KDA PERFORATORIAL PROTEIN) (PERF 15)) (E=1.5e⁻⁴⁵).

NOV32b is predicted expressed in at least the Testis. Expression information was derived from the tissue sources of the sequences that were included in the derivation of the sequence of NOV32b. The sequence is also predicted to be expressed in the estis because of the expression pattern of (GENBANKD: gb:GENBANK-ID:RRU07870|acc:U07870.1) a closely related Rattus norvegicus testis lipid binding protein mRNA, complete cds homolog in Rattus norvegicus.

Homologies to any of the above NOV32a and NOV32b proteins will be shared by the other NOV32 proteins insofar as they are homologous to each other as shown above. Any reference to NOV32 is assumed to refer to NOV32a and NOV32b proteins in general, unless otherwise noted.

NOV32a and NOV32b are very closely homologous as is shown in the amino acid alignment in Table 32E.

[image]

NOV32a also has homology to the amino acid sequences shown in the BLASTP data listed in Table 32F.

TABLE 32F
BLAST results for NOV32a

| Gene Index/ | | Length | Identity | Positives | |
|---|---|---|---|---|---|
| Identifier | Protein/Organism | (aa) | (%) | (%) | Expect |
| gi\|17449600\|ref\| | similar to RIKEN | 132 | 130/132 | 130/132 | 1e−58 |
| XP_070467.1\| | cDNA 1700007P10 | | (98%) | (98%) | |
| (XM_070467) | gene (H. sapiens) | | | | |
| | [Homo sapiens] | | | | |
| gi\|13386216\|ref\| | RIKEN cDNA | 132 | 93/132 | 113/132 | 2e−44 |
| NP_081557.1\| | 1700007P10 [Mus | | (70%) | (85%) | |
| (NM_027281) | musculus] | | | | |
| gi\|6755801\|ref\|N | testis lipid | 132 | 90/132 | 112/132 | 7e−44 |
| P_035728.1\| | binding protein | | (68%) | (84%) | |
| (NM_011598) | [Mus musculus] | | | | |
| gi\|12408304\|ref\| | testis lipid | 132 | 89/132 | 112/132 | 2e−43 |
| NP_074045.1\| | binding protein | | (67%) | (84%) | |
| (NM_022854) | [Rattus | | | | |
| | norvegicus] | | | | |
| gi\|14423683\|sp\|O | Fatty acid-binding | 132 | 84/131 | 111/131 | 3e−41 |
| 97788\|FABA_PIG | protein, adipocyte | | (64%) | (84%) | |
| | (AFABP) (Adipocyte | | | | |
| | lipid-binding | | | | |
| | protein) (ALBP) | | | | |
| | (A-FABP) (AP2) | | | | |

The homology of these sequences is shown graphically in the ClustalW analysis shown in Table 32G.

[image]

Table 32H lists the domain description from DOMAIN analysis results against NOV32. This indicates that the NOV32 sequence has properties similar to those of other proteins known to contain these domains.

TABLE 32H
Domain Analysis of NOV32

gnl|Pfam|pfam00061, lipocalin, Lipocalin/cytosolic fatty-acid binding
protein family. Lipocalins are transporters for small hydrophobic
molecules, such as lipids, steroid hormones, bums, and retinoids.
Alignment subsumes both the lipocalin and fatty acid binding protein
signatures from PROSITE. This is supported on structural and functional
grounds. Structure is an eight-stranded beta barrel.
CD-Length = 145 residues, 87.6% aligned
Score = 57.8 bits (138), Expect = 4e−10

| | | | | |
|---|---|---|---|---|
| NOV32: | 5 | FLGTWKLVSSENFEDYMKE---LGFAARNMAGLVK-PTVTISVDGKMMTIRTESSFQDTK | 60 | (SEQ ID NO:400) |
| | | \| \| \| \|\|+\| \|\|+ +\|\| + \| \| +\| + \| \|\| \| \|+ + \| | | |
| Sbjct: | 2 | FAGKWYLVASANFDPELKEELGVLEATRKEITPLKEGNLEIVFDGDKNGICEETFGKLEK | 61 | (SEQ ID NO:401) |
| NOV32: | 61 | ISFKLGEEFDETTADNRKVKSTITLENGSMIEVQKWLGKETTIKRKIVDEKMVVECKMNN | 120 | |
| | | \|\|\| \|\|\| \| \|\|\| \| +\| ++ \|\|\| \| \|\|+ ++ + + | | |
| Sbjct: | 62 | TK-KLGVEFDYYTGDNRFVVLDTDYDNYLLVCVQKGDGNETSRTAELYGRTPELSPEALE | 120 | |
| N0V32: | 121 | IVSTRIYE | 128 | |
| | | + \| \| | | |
| Sbjct: | 121 | LFETATKE | 128 | |

The fatty acid-binding protein (FABP) family consists of small, cytosolic proteins believed to be involved in the uptake, transport, and solubilization of their hydrophobic ligands. Recently, a number of family members have been identified that are secreted, such as gastrotropin and mammary-derived growth inhibitor. The family is implicated in general lipid metabolism, acting as intracellular transporters of hydrophobic metabolic intermediates and as carriers of lipids between membranes. The family is implicated in general lipid metabolism, acting as intracellular transporters of hydrophobic metabolic intermediates and as carriers of lipids between membranes. Members of this family have highly conserved sequences and tertiary structures, and have probably diverged from a common ancestor. Using an antibody against testis lipid-binding protein, a member of the FABP family, Kingma et al. (1998) identified a protein from bovine retina and testis that coeluted with exogenously added docosahexaenoic acid during purification. Amino acid sequencing and subsequent isolation of its cDNA revealed it to be nearly identical to a bovine protein expressed in the differentiating lens and to be the likely bovine homologue of the human epidermal fatty acid-binding protein (E-FABP). From quantitative Western blot analysis, it was estimated that bovine E-FABP comprised 0.9%, 0.1%, and 2.4% of retina, testis, and lens cytosolic proteins, respectively. Binding studies using the fluorescent probe ADIFAB indicated that this protein bound fatty acids of differing levels of saturation with relatively high affinities. Kd values ranged from 27 to 97 nM. In addition, the protein was immunolocalized to the Muller cells in the retina as well as to Sertoli cells in the testis. The location of bovine E-FABP in cells known to be supportive to other cell types in their tissues and the ability of E-FABP to bind a variety of fatty acids with similar affinities indicate that it may be involved in the uptake and transport of fatty acids essential for the nourishment of the surrounding cell types. See InterPro IPR000463.

The protein similarity information, expression pattern, cellular localization, and map location for the NOV32 protein and nucleic acid disclosed herein suggest that this Testis Lipid Binding Protein-like protein may have important structural and/or physiological functions characteristic of the fatty-acid binding protein family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV32 nucleic acids and proteins of the invention have applications in the diagnosis and/or treatment of various diseases and disorders. For example, the compositions of the present invention will have efficacy for the treatment of patients suffering from: fertility as well as other diseases, disorders and conditions.

These antibodies may be generated according to methods known in the art, using prediction from hydrophobicity charts, as described in the “Anti-NOVX Antibodies” section below. The disclosed NOV32 protein has multiple hydrophilic regions, each of which can be used as an immunogen. In one embodiment, a contemplated NOV32 epitope is from about amino acids 15 to 25. In another embodiment, a contemplated NOV32 epitope is from about amino acids 26 to 28. In other specific embodiments, contemplated NOV32 epitopes are from about amino acids 48 to 50, 52 to 60, 61 to 64, 68 to 71, 76 to 78, 82 to 83, 97 to 98, 99 to 101, 104 to 107, 114 to 116, 118 to 119 and 122 to 124.

NOV33

A disclosed NOV33 (designated CuraGen Acc. No. CG57356-01), which encodes a novel Intracellular Thrombosopondin Domain Containing Protein-like protein and includes the 1238 nucleotide sequence (SEQ ID NO:99) is shown in Table 33A. An open reading frame for the mature protein was identified beginning with an TAC initiation codon at nucleotides 2-4 and ending with a TAA stop codon at nucleotides 1236-1238. Putative untranslated regions are underlined in Table 33b, and the start and stop codons are in bold letters.

TABLE 33A
NOV33 Nucleotide Sequence

(SEQ ID NO:99)

GTACGTGTAGTCCTGAAACCAGCTTTTCTCTCTCCAAAGAAGCACCAAGGGAGCATCTGGACCACCAGGCTGCACA
CCAACCCTTCCCCAGACCGCGATTCCGACAAGAGACGGGGCACCCTTCATTGCAAAGAGATTTCCCCAGATCCTTT
CTCCTTGATCTACCAAACTTTCCAGATCTTTCCAAAGCTGATATCAATGGGCAGAATCCAAATATCCAGGTCACCA
TAGAGGTGGTCGACGGTCCTGACTCTGAAGCAGATAAAGATCAGCATCCGGAGAATAAGCCCAGCTGGTCAGTCCC
ATCCCCCGACTGGCGGGCCTGGTGGCAGAGGTCCCTGTCCTTGGCCAGGGCAAACAGCGGGGACCAGGACTACAAG
TACGACAGTACCTCAGACGACAGCAACTTCCTCAACCCCCCCAGGGGGTGGGACCATACAGCCCCAGGCCACCGGA
CTTTTGAAACCAAAGATCAGCCAGAATATGATTCCACAGATGGCGAGGGTGACTGGAGTCTCTGGTCTGTCTGCAG
CGTCACCTGCGGGAACGGCAACCAGAAACGGACCCGGTCTTGTGGCTACGCGTGCACTGCAACAGAATCGAGGACC
TGTGACCGTCCAAACTGCCCAGGAATTGAAGACACTTTTAGGACAGCTGCCACCGAAGTGAGTCTGCTTGCGGGAA
GCGAGGAGTTTAATGCCACCAAACTGTTTGAAGTTGACACAGACAGCTGTGAGCGCTGGATGAGCTGCAAAAGCGA
GTTCTTAAAGAAGTACATGCACAAGGTGATGAATGACCTGCCCAGCTGCCCCTGCTCCTACCCCACTGAGGTGGCC
TACAGCACGGCTGACATCTTCGACCGCATCAAGCGCAAGGACTTCCGCTGGAAGGACGCCAGCGGGCCCAAGGAGA
AGCTGGAGATCTACAAGCCCACTGCCCGGTACTGCATCCGCTCCATGCTGTCCCTGGAGAGCACCACGCTGGCGGC
ACAGCACTGCTGCTACGGCGACAACATGCAGCTCATCACCAGGGGCAAGGGGGCGGGCACGCCCAACCTCATCGGC
ACCGAGTTCTCCGCGGAGCTCCACTACAAGGTGGACGTCCTGCCCTGGATTATCTGCAAGGGTGACTGGAGCAGGT
ATAACGAGGCCCGGCCTCCCAACAACGGACAGGAGTGCACAGAGAGCCCCTCGGACGAGGACTACATCAAGCAGTT
CCAAGAGGCCAGGGAATATTAA

The disclosed NOV33 nucleic acid sequence maps to chromosome 7 and has 373 of 512 bases (72%) identical to a gb:GENBANK-ID:AF111168|acc:AF111168.2 mRNA from Homo sapiens (Homo sapiens serine palmitoyl transferase, subunit II gene, complete cds; and unknown genes) (E=2.3e⁻⁴⁸).

A disclosed NOV33 polypeptide (SEQ ID NO:100) is 411 amino acid residues in length and is presented using the one-letter amino acid code in Table 33B. The SignalP, Psort and/or Hydropathy results predict that NOV33 does not have a signal peptide and is likely to be localized to the cytoplasm with a certainty of 0.6500. In alternative embodiments, a NOV33 polypeptide is located to the mitochondrial matrix space with a certainty of 0.1000 or the lysosome (lumen) with a certainty of 0.1000.

TABLE 33B
Encoded NOV33 Protein Sequence

(SEQ ID NO:190)

TCSPETSFSLSKEAPREHLDHQAAHQPFPRPRFRQETGHPSLQRDFPRSFLLDLPNFPDLSKADINGQNPNIQ
VTIEVVDGPDSEADKDQHPENKPSWSVPSPDWRAWWQRSLSLARANSGDQDYKYDSTSDDSNFLNPPRGWDHT
APGHRTFETKDQPEYDSTDGEGDWSLWSVCSVTCGNGNQKRTRSCGYACTATESRTCDRPNCPGIEDTFRTAA
TEVSLLAGSEEFNATKLFEVDTDSCERWMSCKSEFLKKYMHKVMNDLPSCPCSYPTEVAYSTADIFDRIKRKD
FRWKDASGPKEKLEIYKPTARYCIRSMLSLESTTLAAQHCCYGDNMQLITRGKGAGTPNLIGTEFSAELHYKV
DVLPWIICKGDWSRYNEARPPNNGQECTESPSDEDYIKQFQEAREY

The NOV33 amino acid sequence was found to have 162 of 164 amino acid residues (98%) identical to, and 163 of 164 amino acid residues (99%) similar to, the 361 amino acid residue ptnr:TREMBLNEW-ACC:CAC16127 protein from Homo sapiens (Human) (BA149118.1 (NOVEL PROTEIN)) (E=3.6e⁻⁸⁹).

NOV33 is predicted expressed in at least the following tissues: lung, testis, and b-cell. Expression information was derived from the tissue sources of the sequences that were included in the derivation of the sequence of NOV33.

NOV33 also has homology to the amino acid sequences shown in the BLASTP data listed in Table 33C.

TABLE 33C
BLAST results for NOV33

| Gene Index/ | | Length | Identity | Positives | |
|---|---|---|---|---|---|
| Identifier | Protein/Organism | (aa) | (%) | (%) | Expect |
| gi\|13374941\|emb\| | bA149I18.1 (novel | 391 | 389/391 | 390/391 | 0.0 |
| CAC16127.2\| | protein) [Homo | | (99%) | (99%) | |
| (AL133463) | sapiens] | | | | |
| gi\|4186183\|gb\|AA | unknown [Homo | 658 | 178/392 | 238/392 | 5e−82 |
| D09622.1\| | sapiens] | | (45%) | (60%) | |
| (AF111168) | | | | | |
| gi\|17389974\|gb\|A | Unknown (protein | 151 | 149/151 | 150/151 | 6e−82 |
| AH17997.1\|AAH179 | for IMAGE: 4252124) | | (98%) | (98%) | |
| 97 (BC017997) | [Homo sapiens] | | | | |
| gi\|13559287\|emb\| | dJ1077I2.1 (novel | 60 | 49/49 | 49/49 | 3e−20 |
| CAC36074.1\| | protein) [Homo | | (100%) | (100%) | |
| (AL050320) | sapiens] | | | | |
| gi\|4502359\|ref\|N | brain-specific | 1522 | 28/66 | 36/66 | 6e−05 |
| P_001695.1\| | angiogenesis | | (42%) | (54%), | |
| (NM_001704) | inhibitor 3 [Homo | | | Gaps = | |
| | sapiens] | | | 10/65 | |
| | | | | (15%) | |

The homologous regions of these sequences is shown graphically in the ClustalW analysis shown in Table 33D.

[image]

[image]

Table 33E lists the domain description from DOMAIN analysis results against NOV33. This indicates that the NOV33 sequence has properties similar to those of other proteins known to contain these domains.

TABLE 33E
Domain Analysis of NOV33

gnl|Smart|smart00209, TSP1, Thrombospondin type 1 repeats; Type 1 repeats in
thrombospondin-1 bind and activate TGF-beta.
CD-Length = 51 residues, 98.0% aligned
Score = 47.4 bits (iii), Expect = 2e−06

| | | | | |
|---|---|---|---|---|
| N0V33: | 168 | GDWSLWSVCSVTCGNGNQKRTRSC------GYACT--ATESRTCDRPNCP | 209 | (SEQ ID NO:406X) |
| | | \|+\|\| \|\| \|\|\|\|\|\| \| \| \|\|\| \| \| \|\| \|\|+\| \|+ \|\| | | |
| Sbjct: | 2 | GEWSEWSPCSVTCGGGVQTRTRCCNPPPNGGGPCTGPDTETRACNEQPCP | 51 | (SEQ ID NO:407) |

gnl|Pfam|pfam00090, tsp_1, Thrombospondin type 1 domain.
CD-Length = 48 residues, 100.0% aligned
Score = 43.9 bits (102), Expect = 2e−05

| | | | | |
|---|---|---|---|---|
| N0V33: | 168 | GDWSLWSVCSVTCGNGNQKRTRSC-----GYACT--ATESRTCDRPNC | 208 | (SEQ ID NO:408) |
| | | \|\| \|\| \|\|\|\|\|\| \| + \| \|+\| \| \|\| \| \|+ \| \| | | |
| Sbjct: | 1 | SPWSEWSPCSVTCGKGIRTRQRTCNSPAGGKPCTGDAQETEACMMDPC | 48 | (SEQ ID NO:409) |

The thrombospondin type 1 repeat was first described in 1986 by Lawler & Hynes. It was found in the thrombospondin protein where it is repeated 3 times. Now a number of proteins involved in the complement pathway (properdin, C6, C7, C8A, C8B, C9) as well as extracellular matrix protein like mindin, F-spondin, SCO-spondin and even the circumsporozoite surface protein 2 and TRAP proteins of Plasmodium contain one or more instance of this repeat. It has been involved in cell-cell interaction, inhibition of angiogenesis and apoptosis. The intron-exon organization of the properdin gene confirms the hypothesis that the repeat might have evolved by a process involving exon shuffling. A study of properdin structure provides some information about the structure of the thrombospondin type I repeat. See InterPro 1PR000884.

The protein similarity information, expression pattern, cellular localization, and map location for the NOV33 protein and nucleic acid disclosed herein suggest that this novel intracellular thrombospondin domain containing protein-like protein may have important structural and/or physiological functions characteristic of the novel intracellular thrombospondin domain containing protein family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV33 nucleic acids and proteins of the invention have applications in the diagnosis and/or treatment of various diseases and disorders. For example, the compositions of the present invention will have efficacy for the treatment of patients suffering from: systemic lupus erythematosus, autoimmune disease, asthma, emphysema, scieroderma, allergy, ARDS; fertility, hypogonadism; immunological disease and disorders as well as other diseases, disorders and conditions.

These antibodies may be generated according to methods known in the art, using prediction from hydrophobicity charts, as described in the “Anti-NOVX Antibodies” section below. The disclosed NOV33 protein has multiple hydrophilic regions, each of which can be used as an immunogen. In one embodiment, a contemplated NOV33 epitope is from about amino acids 10 to 40. In another embodiment, a contemplated NOV33 epitope is from about amino acids 55 to 60. In other specific embodiments, contemplated NOV33 epitopes are from about amino acids 90 to 102, 110 to 140, 145 to 155, 190 to 195, 202 to 205, 240 to 255, 260 to 305, 330 to 360 and 370 to 405.

NOV34

One NOVX protein of the invention, referred to herein as NOV34, includes three Ornithine Decarboxylase-like proteins. The disclosed proteins have been named NOV34a, NOV34b and NOV34c.

NOV34a

A disclosed NOV34a (designated CuraGen Acc. No. CG57258-01), which encodes a novel Ornithine Decarboxylase-4-like protein and includes the 1463 nucleotide sequence (SEQ ID NO:101) is shown in Table 34A. An open reading frame for the mature protein was identified beginning with an ATG initiation codon at nucleotides 51-53 and ending with a TGA stop codon at nucleotides 1413-1415. Putative untranslated regions are underlined in Table 34A, and the start and stop codons are in bold letters.

TABLE 34A
NOV34a Nucleotide Sequence

(SEQ ID NO:101)

GGCGGCTGCAGCAGCGGCTCCATCCAGCCCGTCAGCTCCTCCTGCAAGGCATGGCTGGCTACCTGAGTGAATCGGA
CTTTGTGATGGTGGAGGAGGGCTTCAGTACCCGAGACCTGCTGAAGGAACTCACTCTGGGGGCCTCACAGGACGAG
GTAGCTGCCTTCTTCGTGGCTGACCTGGGTGCCATAGTGAGGAAGCACTTTTGCTTTCTGAAGTGCCTGCCACGAG
TCCGGCCCTTTTATGCTGTCAAGTGCAACAGCAGCCCAGGTGTGCTGAAGGTTCTGGCCCAGCTGGGGCTGGGCTT
TAGCTGTGCCAACAAGGCAGAGATGGAGTTGGTCCAGCATATTGGAATCCCTGCCAGTAAGATCATCTGCGCCAAC
CCCTGTAAGCAAATTGCACAGATCAAATATGCTGCCAAGCATGGGATCCAGCTGCTGAGCTTTGACAATGAGATGG
AGCTGGCAAAGGTGGTAAAGAGCCACCCCAGTGCCAAGATGGTTCTGTGCATTGCTACCGATGACTCCCACTCCCT
GAGCTGCCTGAGCCTAAAGTTTGGAGTGTCACTGAAATCCTGCAGACACCTGCTTGAAAATGCGAAGAAGCACCAT
GTGGAGGTGGTGGGTGTGAGTTTTCACATTGGCAGTGGCTGTCCTGACCCTCAGGCCTATGCTCAGTCCATCGCAG
ACGCCCGGCTCGTGTTTGAAATGGGCACCGAGCTGGGTCACAAGATGCACGTTCTGGACCTTGGTGGTGGCTTCCC
TGGCACAGAAGGGGCCAAAGTGAGATTTGAAGAGATTGCTTCCGTGATCAACTCAGCCTTGGACCTGTACTTCCCA
GAGGGCTGTGGCGTGGACATCTTTGCTGAGCTGGGGCGCTACTACGTGACCTCGGCCTTCACTGTGGCAGTCAGCA
TCATTGCCAAGAAGGAGGTTCTGCTAGACCAGCCTGGCAGGGAGGAGGAAAATGGTTCCACCTCCAAGACCATCGT
GTACCACCTTGATGAGGGCGTGTATGGGATCTTCAACTCAGTCCTGTTTGACAACATCTGCCCTACCCCCATCCTG
CAGAAGAAACCATCCACGGAGCAGCCCCTGTACAGCAGCAGCCTGTGGGGCCCGGCGGTTGATGGCTGTGATTGCG
TGGCTGAGGGCCTGTGGCTGCCGCAACTACACGTAGGGGACTGGCTGGTCTTTGACAACATGGGCGCCTACACTGT
GGGCATGGGTTCCCCCTTTTGGGGGACCCAGGCCTGCCACATCACCTATGCCATGTCCCGGGTGGCCTGGCGAAGG
CAGCTGATGGCTGCAGAACAGGAGGATGACGTGGAGGGTGTGTGCAAGCCTCTGTCCTGCGGCTGGGAGATCACAG
ACACCCTGTGCGTGGGCCCTGTCTTCACCCCAGCGAGCATCATGTGAGTGGGCCTCGTTCCCCCCGGAGAATCCCA
GCGGGGCCTCAGAGATGCA

The disclosed NOV34 nucleic acid sequence maps to chromosome 1 and has 948 of 1373 bases (69%) identical to a gb:GENBANK-ID:AF217544|acc:AF217544.2 mRNA from Xenopus laevis (Xenopus laevis ornithine decarboxylase-2 mRNA, complete cds) (E=9.8e⁻¹¹⁰).

The NOV34 polypeptide (SEQ ID NO:102) is 454 amino acid residues in length and is presented using the one-letter amino acid code in Table 34B. The SignalP, Psort and/or Hydropathy results predict that NOV34a does not have a signal peptide and is likely to be localized to the cytoplasm with a certainty of 0.4500. In alternative embodiments, a NOV34 polypeptide is located to the microbody (peroxisome) with a certainty of 0.4387, the mitochondrial matrix space with a certainty of 0.1000, or the lysosome (lumen) with a certainty of 0.1000.

TABLE 34B
Encoded NOV34a Protein Sequence

(SEQ ID NO:102)

MAGYLSESDFVMVEEGFSTRDLLKELTLGASQDEVAAFFVADLGAIVRKHFCFLKCLPRVRPFYAVKCNSSPG
VLKVLAQLGLGFSCANKAEMELVQHIGIPASKIICANPCKQIAQIKYAAKHGIQLLSFDNEMELAKVVKSHPS
AKMVLCIATDDSHSLSCLSLKFGVSLKSCRHLLENAKKHHVEVVGVSFHIGSGCPDPQAYAQSIADARLVFEM
GTELGHKMHVLDLGGGFPGTEGAKVRFEEIASVINSALDLYFPEGCGVDIFAELGRYYVTSAFTVAVSIIAKK
EVLLDQPGREEENGSTSKTIVYHLDEGVYGIFNSVLFDNICPTPILQKKPSTEQPLYSSSLWGPAVDGCDCVA
EGLWLPQLHVGDWLVFDNMGAYTVGMGSPFWGTQACHITYAMSRVAWRRQLMAAEQEDDVEGVCKPLSCGWEI
TDTLCVGPVFTPASIM

A disclosed NOV34a amino acid sequence was found to have 277 of 456 amino acid residues (60%) identical to, and 353 of 456 amino acid residues (77%) similar to, the 456 amino acid residue ptnr:SPTREMBL-ACC:Q918S4 protein from Xenopus laevis (African clawed frog) (ORNITHINE DECARBOXYLASE-2) (E=3.4e⁻¹⁴⁸).

NOV34a is expressed in at least the following tissues: Bone Marrow, Lymph node, Prostate, Right Cerebellum, and Substantia Nigra. Expression information was derived from the tissue sources of the sequences that were included in the derivation of the sequence of NOV34.

NOV34b

A disclosed NOV34b (designated CuraGen Acc. No. CG57258-02), which encodes a novel Ornithine Decarboxylase-like protein and includes the 1613 nucleotide sequence (SEQ ID NO:103) is shown in Table 34C. An open reading frame for the mature protein was identified beginning with an ATG initiation codon at nucleotides 4244 and ending with a TGA stop codon at nucleotides 1248-1250. Putative untranslated regions are underlined in Table 34C, and the start and stop codons are in bold letters.

TABLE 34C
NOV34b Nucleotide Sequence

(SEQ ID NO:193)

AGCAGCGGCTCCATCCAGCCCGTCAGCTCCTCCTGCAAGGCATGGCTGGCTACCTGAGTGAATCGGACTTTGTGA
TGGTGGAGGAGGGCTTCAGTACCCGAGACCTGCTGAAGGAACTCACTCTGGGGGCCTCACAGGCCACCACGGCAG
AGATGGAGTTGGTCCAGCATATTGGAATCCCTGCCAGTAAGATCATCTGCGCCAACCCCTGTAAGCAAATTGCAC
AGATCAAATATGCTGCCAAGCATGGGATCCAGCTGCTGAGCTTTGACAATGAGATGGAGCTGGCAAAGGTGGTAA
AGAGCCACCCCAGTGCCAAGATGGTTCTGTGCATTGCTACCGATGACTCCCACTCCCTGAGCTGCCTGAGCCTAA
AGTTTGGAGTGTCACTGAAATCCTGCAGACACCTGCTTGAAAATGCGAAGAAGCACCATGTGGAGGTGGTGGGTG
TGAGTTTTCACATTGGCAGTGGCTGTCCTGACCCTCAGGCCTATGCTCAGTCCATCGCAGACGCCCGGCTCGTGT
TTGAAATGGGCACCGAGCTGGGTCACAAGATGCACGTTCTGGACCTTGGTGGTGGCTTCCCTGGCACAGAAGGGG
CCAAAGTGAGATTTGAAGAGATTGCTTCCGTGATCAACTCAGCCTTGGACCTGTACTTCCCAGAGGGCTGTGGCG
TGGACATCTTTGCTGAGCTGGGGCGCTACTACGTGACCTCGGCCTTCACTGTGGCAGTCAGCATCATTGCCAAGA
AGGAGGTTCTGCTAGACCAGCCTGGCAGGGAGGAGGAAAATGGTTCCACCTCCAAGACCATCGTGTACCACCTTG
ATGAGGGCGTGTATGGGATCTTCAACTCAGTCCTGTTTGACAACATCTGCCCTACCCCCATCCTGCAGAAGAAAC
CATCCACGGAGCAGCCCCTGTACAGCAGCAGCCTGTGGGGCCCGGCGGTTGATGGCTGTGATTGCGTGGCTGAGG
GCCTGTGGCTGCCGCAACTACACGTAGGGGACTGGCTGGTCTTTGACAACATGGGCGCCTACACTGTGGGCATGG
GTTCCCCCTTTTGGGGGACCCAGGCCTGCCACATCACCTATGCCATGTCCCGGGTGGCCTGGGAAGCGCTGCGAA
GGCAGCTGATGGCTGCAGAACAGGAGGATGACGTGGAGGGTGTGTGCAAGCCTCTGTCCTGCGGCTGGGAGATCA
CAGACACCCTGTGCGTGGGCCCTGTCTTCACCCCAGCGAGCATCATGTGAGTGGGCCTCGTTCCCCCCGGAGAAT
CCCAGCGGGGCCTCAGAGATGCATCTGGGAGAGGTGGGGAAGATGGCAGGCAAGGGTACCCTTGGCCAGGACTCT
GGTGCCCACCCTGCCACCCCCGCGCTCCACCTGCAGTGTTTCTGCCCTGTAAATAGGACCAGTCTTACACTCGCT
GTAGTTCAAGTATGCAACATAAATCCTGTTCCTTCCAGCTGTGTCTGCCTCCTCTGCAGTGCAAGGGGCCTGGTC
AGCCAGGTGTGGGGGTGTTCTTGGGGTCTCCTTTGGTCTCCTTCCCACCTTTGTAAATATAATGCAAATAAATAA
ATATTTAGGTTTTTAAAAACTGAAAAAAAAAAAAAAAA

The disclosed NOV34b nucleic acid sequence maps to chromosome 1 and has 1482 of 1489 bases (99%) identical to a gb:GENBANK-ID:BC010449|acc:BC010449.1 mRNA from Homo sapiens (Homo sapiens, Similar to ornithine decarboxylase 1, clone MGC: 18232 IMAGE:4156927, mRNA, complete cds) (E=0.0).

A disclosed NOV34b polypeptide (SEQ ID NO:104) is 402 amino acid residues in length and is presented using the one-letter amino acid code in Table 34D. The SignalP, Psort and/or Hydropathy results predict that NOV34b does not have a signal peptide and is likely to be localized to the cytoplasm with a certainty of 0.4500. In alternative embodiments, a NOV34b polypeptide is located to the microbody (peroxisome) with a certainty of 0.4154, the mitochondrial matrix space with a certainty of 0.1000 or the lysosome (lumen) with a certainty of 0.1000.

TABLE 34D
Encoded NOV34b Protein Sequence

(SEQ ID NO:194)

MAGYLSESDFVMVEEGFSTRDLLKELTLGASQATTAEMELVQHIGIPASKIICANPCKQIAQIKYAAKHGIQLLSF
DNEMELAKVVKSHPSAKMVLCIATDDSHSLSCLSLKFGVSLKSCRHLLENAKKHHVEVVGVSFHIGSGCPDPQAYA
QSIADARLVFEMGTELGHKMHVLDLGGGFPGTEGAKVRFEEIASVINSALDLYFPEGCGVDIFAELGRYYVTSAFT
VAVSIIAKKEVLLDQPGREEENGSTSKTIVYHLDEGVYGIFNSVLFDNICPTPILQKKPSTEQPLYSSSLWGPAVD
GCDCVAEGLWLPQLHVGDWLVFDNMGAYTVGMGSPFWGTQACHITYAMSRVAWEALRRQLMAAEQEDDVEGVCKPL
SCGWEITDTLCVGPVFTPASIM

The NOV34b amino acid sequence was found to have 373 of 381 amino acid residues (97%) identical to, and 375 of 381 amino acid residues (98%) similar to, the 460 amino acid residue ptnr:TREMBLNEW-ACC:AAH10449 protein from Homo sapiens (Human) (SIMILAR TO ORNITHINE DECARBOXYLASE 1) (E=4.1 e⁻²⁰³).

NOV34b is expressed in at least the following tissues: Brain, Lung, Heart, Pineal Gland, Colon, Peripheral Blood, Lymphoid tissue, Bone Marrow, Lymph node, Prostate, Right Cerebellum, and Substantia Nigra. Expression information was derived from the tissue sources of the sequences that were included in the derivation of the sequence of CuraGen Acc. No. CG57258-O₂. The sequence is also predicted to be expressed in the Brain because of the expression pattern of (GENBANK-ID: gb:GENBANK-ID:BC010449|acc:BC010449.1), a closely related Homo sapiens, Similar to ornithine decarboxylase 1, clone MGC:18232 IMAGE:4156927, mRNA, complete cds homolog in species Homo sapiens.

NOV34c

A disclosed NOV34c (designated CuraGen Acc. No. CG57258-03), which encodes a novel Ornithine Decarboxylase-like protein and includes the 679 nucleotide sequence (SEQ ID NO:105) is shown in Table 34E. An open reading frame for the mature protein was identified beginning with an ATG initiation codon at nucleotides 23-25 and ending with a TGA stop codon at nucleotides 677-679. Putative untranslated regions are underlined in Table 34E, and the start and stop codons are in bold letters.

TABLE 34E
NOV34c Nucleotide Sequence

(SEQ ID NO:105)

CCGTCAGCTCCTCCTGCAAGGCATGGCTGGCTACCTGAGCGAATCGGACTTTGTGATGGTGGAGGAGGGCTTCA
GTACCCGAGACCTGCTGAAGGAACTCACTCTGGGGGCCTCACAGGCCACCACGGACGAGGTAGCTGCCTTCTTC
GTGGCTGACCTGGGTGCCATAGTGAGGAAGCACTTTTGCTTTCTGAAGTGCCTGCCACGAGTCCGGCCCTTTTA
TGCTGTCAAGTGCAACAGCAGCCCAGGTGTGCTGAAGGTTCTGGCCCAGCTGGGGCTGGGCTTTAGCTGTGCCA
ACATCTGCCCTACCCCCATCCTGCAGAAGAAACCATCCACGGAGCAGCCCCTGTACAGCAGCAGCCTGTGGGGC
CCGGCGGTTGATGGCTGTGATTGCGTGGCTGAGGGCCTGTGGCTGCCGCAACTACACGTAGGGGACTGGCTGGT
CTTTGACAACATGGGCGCCTACACTGTGGGCATGGGTTCCCCCTTTTGGGGGACCCAGGCCTGCCACATCACCT
ATGCCATGTCCCGGGTGGCCTGGGAAGCGCTGCGAAGGCAGCTGATGGCTGCAGAACAGGAGGATGACGTGGAG
GGTGTGTGCAAGCCTCTGTCCTGCGGCTGGGAGATCACAGACACCCTGTGCGTGGGCCCTGTCTTCACCCCAGC
GAGCATCATGTGA

The disclosed NOV34c nucleic acid sequence maps to chromosome 1 and has 388 of 390 bases (99%) identical to a gb:GENBANK-ID:BC010449|acc:BC010449.1 mRNA from Homo sapiens (Homo sapiens, Similar to ornithine decarboxylase I, clone MGC: 18232 IMAGE:4156927, mRNA, complete cds) (E=2.3e⁻¹⁴⁶).

A disclosed NOV34c polypeptide (SEQ ID NO:106) is 218 amino acid residues in length and is presented using the one-letter amino acid code in Table 34F. The SignalP, Psort and/or Hydropathy results predict that NOV34c does not have a signal peptide and is likely to be localized to the microbody (peroxisome) with a certainty of 0.4748. In alternative embodiments, a NOV34c polypeptide is located to the cytoplasm with a certainty of 0.4500, the mitochondrial matrix space with a certainty of 0.1000, or the lysosome (lumen) with a certainty of 0.1000.

TABLE 34F
Encoded NOV34c Protein Sequence

(SEQ ID NO:106)

MAGYLSESDFVMVEEGFSTRDLLKELTLGASQATTDEVAAFFVADLGAIVRKHFCFLKCLPRVRPFYAVKCNSSP
GVLKVLAQLGLGFSCANICPTPILQKKPSTEQPLYSSSLWGPAVDGCDCVAEGLWLPQLHVGDWLVFDNMGAYTV
GMGSPFWGTQACHITYAMSRVAWEALRRQLMAAEQEDDVEGVCKPLSCGWEITDTLCVGPVFTPASIM

The NOV34c amino acid sequence was found to have 127 of 127 amino acid residues (100%) identical to, and 127 of 127 amino acid residues (100%) similar to, the 460 amino acid residue ptnr:TREMBLNEW-ACC:AAH10449 protein from Homo sapiens (Human) (SIMILAR TO ORNITHINE DECARBOXYLASE 1) (E=9.1e⁻¹¹⁸).

NOV34c is expressed in at least the following tissues: Brain, Lung, Heart, Pineal Gland, Colon, Peripheral Blood, Lymphoid tissue, Bone Marrow, Lymph node, Prostate, Right Cerebellum, and Substantia Nigra. Expression information was derived from the tissue sources of the sequences that were included in the derivation of the sequence of CuraGen Acc. No. CG57258-03. The sequence is predicted to be expressed in the brain because of the expression pattern of (GENBANK-ID: gb:GENBANK-ID:BC010449|acc:BC010449.1) a closely related Homo sapiens, Similar to ornithine decarboxylase 1, clone MGC: 18232 IMAGE:4156927, mRNA, complete cds homolog in species Homo sapiens.

Homologies to any of the above NOV34a, NOV34b and NOV34c proteins will be shared by other NOV34 proteins insofar as they are homologous to each other as shown below. Any reference to NOV34 is assumed to refer to NOV34a, NOV34b and NOV34c proteins in general, unless otherwise noted.

NOV34a, NOV34b and NOV34c are very closely homologous as is shown in the amino acid alignment in Table 34G.

[image]

[image]

NOV34a also has homology to the amino acid sequences shown in the BLASTP data listed in Table 34H.

TABLE 34H
BLAST results for NOV34a

| Gene Index/Identifier | Protein/Organism | Length (aa) | Identity (%) | Positives (%) | Expect |
|---|---|---|---|---|---|
| gi\|16506287\|ref\| | hypothetical | 460 | 454/460 | 454/460 | 0.0 |
| NP_443724.1\| | protein | | (98%) | (98%) | |
| (NM_052998) | XP_054282; | | | | |
| | hypothetical gene | | | | |
| | supported by | | | | |
| | BC010449; ODC- | | | | |
| | paralog [Homo | | | | |
| | sapiens] | | | | |
| gi\|17444708\|ref\| | similar to | 480 | 454/480 | 454/480 | 0.0 |
| XP_054282.2\| | ornithine | | (94%) | (94%) | |
| (XM_054282) | decarboxylase- | | | | |
| | like protein | | | | |
| | variant 2 (H. | | | | |
| | sapiens) [Homo | | | | |
| | sapiens] | | | | |
| gi\|16552627\|dbj\| | unnamed | 365 | 362/365 | 362/365 | 0.0 |
| BAB71356.1\| | protein | | (99%) | (99%) | |
| (AK057051) | product [Homo | | | | |
| | sapiens] | | | | |
| gi\|15858869\|gb\| | ornithine | 362 | 343/354 | 343/354 | 0.0 |
| AAL08052.1\| | decarboxylase- | | (96%) | (96%) | |
| (AY050637) | like protein | | | | |
| | variant 3 [Homo | | | | |
| | sapiens] | | | | |
| gi\|15858867\|gb\| | ornithine | 374 | 343/366 | 343/366 | 0.0 |
| AAL08051.1\| | decarboxylase- | | (93%) | (93%) | |
| (AY050636) | like protein | | | | |
| | variant 4 [Homo | | | | |
| | sapiens] | | | | |

The homology of these sequences is shown graphically in the ClustalW analysis shown in Table 34I.

[image]

[image]

Tables 34J and 34K list the domain description from DOMAIN analysis results against NOV34. This indicates that the NOV34 sequence has properties similar to those of other proteins known to contain these domains.

TABLE 34J
Domain Analysis of NOV34a

gnl|Pfam|pfam02784, Orn_Arg_deC_N, Pyridoxal-dependent decarboxylase, pyridoxal
binding domain. These pyridoxal-dependent decarboxylases acting on ornithine,
lysine, R and related substrates This domain has a TIM barrel fold.
CD-Length = 246 residues, 99.2% aligned
Score = 248 hits (634), Expect = 4e−67

| | | | | |
|---|---|---|---|---|
| NOV34: | 42 | DLGAIV-RKHFCFLKCLPRVRPFYAVKCNSSPGVLKVLAQLGLGFSCANKAEMELVQHIG | 100 | (SEQ ID NO:415) |
| | | \|\|\| \|\| \| \| + \|\|\|++\|\|\|\|\|\| \|\| \| \|\|++\|\|+\|\| \|\| \|\|+\| \|+\| \| G | | |
| Sbjct: | 1 | DLGLIVRRIHALWQAFLPRIQPFYAVKANSDPAVLRLLAELGTGFDCASKGELERVLAAG | 60 | (SEQ ID NO:416) |
| NOV34: | 101 | IPASKIICANPCKQIAQIKYAAKHGIQLLSFDNEMELAKVVKSHPSAKMVLCIATDDSHS | 160 | |
| | | +\| +\|\| \|\|\|\|\| ++++\|\| +\|\|+\|\|++ \|\| \|\| \|+ + \| \|+++\| + \| | | |
| Sbjct: | 61 | VPPERIIFANPCKDRSELRYALEHGVVCVTVDNVEELEKLARLAPEARLLLRVKPDVDAH | 120 | |
| NOV34: | 161 | LSC-----LSLKFGVSLKSCRHLLENAKKHHVEVVGVSFHIGSGCPDPQAYAQSIADARL | 215 | |
| | | \| \|\|\| \|+ \|\|+ \|\|+ + \|\|\|\| \|\|+\|\|\|\| \| +\|+ ++ \|\|\| | | |
| Sbjct: | 121 | AHCYLSTGQDSKFGADLEEAEALLKAAKELGLNVVGVHFHVGSGCTDAEAFVKAARDARN | 180 | |
| NOV34: | 216 | VFEMGT-ELGHKMHVLDLGGGFPGTEGAKVRFEEIASVINSALDLYFPEGCGVDIFAELG | 274 | |
| | | \|\|+ \| \|\|\| ++ +\|\|\|\|\|\|\| \|\|\| \| \|\|\|+\|\|+ \|\| \| \|\| \| | | |
| Sbjct: | 181 | VFDQGADELGFELKILDLGGGFGVDYTGAEDFEEYAEVINAALEEVFPHDPHPTIIAEPG | 240 | |
| NOV34: | 275 | RYYV 278 | | |
| | | \|\| \| | | |
| Sbjct: | 241 | RYIV 244 | | |

gnl|Pfam|pfam00278, Orn_DAP_Arg_deC, Pyridoxal-dependent decarboxylase, C-
terminal sheet domain. These pyridoxal-dependent decarboxylases act on
ornithine, lysine, R and related substrates.
CD-Length = 119 residues, 89.9% aligned
Score = 89.7 bits (221), Expect = 3e−19

| | | | | |
|---|---|---|---|---|
| NOV34: | 283 | VAVSIIAKKEVLLDQPGREEENGSTSKTIVYHLDEGVYGIFNSVLFDNICPTPILQKKP | 342 | (SEQ ID NO:417) |
| | | \|+ ++\|\|\|\| \| ++ \| +\|++++\| \| \| \| + \| +\| ++ | | |
| Sbjct: | 1 | TLVSNVIAKKTV------PSDDEDGKDDTRMYYVNDGGYSSFIRPLLYHAHPHALLLRRS | 54 | (SEQ ID NO:418) |
| NOV34: | 343 | STEQPLYSSSLWGPAVDGCDCVAEGLWLPQLHVGDWLVFDNMGAYTVGMGSPF 395 | | |
| | | \|+\| \|\|+\|\|\| \| \| + + \|\|+\| \|\|\|\|\| \| + \|\|\|\| \| \| \| | | |
| Sbjct: | 55 | LDEEPPRKSSIWGPTCDSLDKIIKDRLLPELDVGDWLAFFDTGAYTEAMASNF 107 | | |

TABLE 34K
Domain Analysis of NOV34a

Orn_DAP_Arg_deC (InterPro) Pyridoxal-dependent decarboxylase 430.6 6.2e−128
1
Parsed for domains:

| | | seq | seq | hmm | hmm | | |
|---|---|---|---|---|---|---|---|
| Model | Domain | from | to | from | to | score | E-value |
| Orn_DAP_Arg_deC: | 1/1 | 38 | 398 . . . | 1 | 467 [] | 430.6 | 6.2e−128 |

Alignments of top-scoring domains:
Orn_DAP_Arg_deC: domain 1 of 1, from 38 to 398: score 430.6, E=6.2e-128

| | | | | |
|---|---|---|---|---|
| | | *->fyvyDlglHivrrihalwkaflprgqynsvvkpfYAVKansdpavlr | | (SEQ ID NO:419) |
| | | \|+\|+\|\|\| ++\|+\| + ++\|\|\| \|+\|\|\|\|\|\|+\|\|+\|+\|\|+ | | |
| NOV34A | 38 | FFVADLG--AIVRKHFCFLKCLPR------VRPFYAVKCNSSPGVLK | 76 | (SEQ ID NO:420) |
| | | lLaelGtHslGfDcaSkgELerVLaaylagvsPerIifanpcKsrselry | | |
| | | +\|\|+\|\|+ \|\| \|\| \|+\| \|+\|+ +\|+++++\|\| \|\|\|\|\|+ ++++\| | | |
| NOV34A | 77 | VLAQLGL---GFSCANKAEMELVQH---IGIPASKIICANPCKQIAQIKY | 120 | |
| | | AlehrkMGgvvcvtvDnveELekiaklapeaGvkprllLRvkpdvdahah | | |
| | | \|+ \| \|++ +\|\|+ \|\|+\|+ \| +\| \| +++\| +++\| \|+\|+ | | |
| NOV34A | 121 | AAKH----GIQLLSFDNEMELAKVVKSHPSA----KMVLCIATD-DSHSL | 161 | |
| | | crlstGqedsKFGadledgedaealLkaAkelgnlnvvGvhFHVGSgisd | | |
| | | + \|\| \|\|\|++\|++ ++ \|\|+ \|\|+++ ++\|\|\|\|+\|\|+\|\|\|++\| | | |
| NOV34A | 162 | SCLSL-----KFGVSLKS---CRHLLENAKKHH-VEVVGVSFHIGSGCPD | 202 | |
| | | leafvkAvrdarnvfdqgadelGfktidlkiLDiGGGfgvdytgtrsqSD | | |
| | | ++\|+++ ++\|\|\| \|\|++\| \|\|\|+\| +++\|\|+\|\|\|\|++++ + | | |
| | | mSVaedfeeiAevinaaleelfphagygdpgptiiaEPGRyivAaagtLv | | |
| | | ++ \|\|\|\|\|+\|\|\| \|\|+++\|\|+ ++\|++\| \|\| \|\|\|+\| +\|+\| + | | |
| NOV34A | 243 | ---KVRFEEIASVINSALDLYFPE----GCGVDIFAELGRYYVTSAFTVA | 285 | |
| | | snViakkevpsddadttsdslreeskDdtrmyyvnDggygsfirpllyha | | |
| | | + +\|\|\|\|\|\| \|+ +++ + \|+ +\| \|++ \|+\|\| \|+ \|+++ | | |
| NOV34A | 286 | VSIIAKKEVLLDQ--PGREEE-NGSTSKTIVYHLDEGVYGIFNSVLFDNI | 332 | |
| | | hpealllrrggevqyqdaeteraadkslsnFsLfqsyPdAwgidqLfPvl | | |
| | | \|++ \|+++ | | |
| NOV34A | 333 | CPTPILQKK----------------------------------------- | 341 | |
| | | PlrsldeepkrkssivGptCDsDGklDkiikddGiaedrllPelkpvGDw | | |
| | | + \| \| ++\|\|++\|\| \|+ \|++++ + \|\| \|+ \|\|\|\| | | |
| NOV34A | 342 | ---PSTEQPLYSSSLWGPAVDG---CDCVAE------GLWLPQLH-VGDW | 378 | |
| | | LafpdtGAYtyamasnyNgF<-* | | |
| | | \|+\| ++\|\|\|\|+ \|+\| + \| | | |
| NOV34A | 379 | LVFDNMGAYTVGMGSPFWGT 398 | | |

These enzymes are collectively known as group IV decarboxylases. Pyridoxal-dependent decarboxylases acting on ornithine, lysine, arginine and related substrates can be classified into two different families on the basis of sequence similarities. Members of this family while most probably evolutionary related, do not share extensive regions of sequence similarities. The proteins contain a conserved lysine residue which is known, in mouse ODC, to be the site of attachment of the pyridoxal-phosphate group. The proteins also contain a stretch of three consecutive glycine residues and has been proposed to be part of a substrate-binding region. See InterPro IPR000183 and IPR002432, (Orn_DAP_Arg decarbxylse).

Ornithine decarboxylase (ODC) is a key enzyme in polyamine biosynthesis. Turnover of ODC is extremely rapid and highly regulated, and is accelerated when polyamine levels increase (Murakami et al., (2000). Biochem Biophys Res Commun 267(1):1-6, PMID: 10623564). Expression and activity of ornithine decarboxylase directly correlates with the proliferation stage of cells. Ornithine decarboxylase is transcriptionally induced by tumor promoter TPA (Nguyen-Ba and Vasseur (1999). Oncol Rep 6(4):925-32. PMID: 10373683). It has also been shown to be transactivated by the c-myc oncogene in certain cell/tissue types and to cooperate with the ras oncogene in malignant transformation of epithelial tissues (Fuhnnann et al., (1999). Mutat Res 437(3):205-17. PMID: 10592328; Reddy (1999). J Nutr 1129(7 Suppl):1478S-82S. PMID: 1039562; Nguyen-Ba and Vasseur (1999). Oncol Rep 6(4):925-32. PMID: 10373683). Furthermore, inhibition of colon carcinogenesis was associated with a decrease in colonic mucosal cell proliferation and activities of colonic mucosal and tumor ornithine decarboxylase and ras-p21 (Reddy (1999). J Nutr 1129(7 Suppl): 1478S-82S. PMID: 1039562;). The rationale for the inhibition of ornithine decarboxylase as a cancer chemopreventive agent has been strengthened in recent years. Recent clinical cancer chemoprevention trials have demonstrated that DFMO, which is an inhibitor of ornithine decarboxylase, can be given over long periods of time at low doses that suppress polyamine contents in gastrointestinal and other epithelial tissues but cause no detectable hearing loss or other side effects (Meyskens and Gerner (1999). Clin Cancer Res 5(5):945-51. PMID: 10353725). Clinical chemoprevention trials are also in progress to investigate the efficacy of DFMO to suppress surrogate end point biomarkers (e.g., colon polyp recurrence) of carcinogenesis in patient populations at elevated risk for the development of specific epithelial cancers, including colon, esophageal, breast, cutaneous, and prostate malignancies (Meyskens and Gerner (1999). Clin Cancer Res 5(5):945-51. PMID: 10353725). Therefore, the novel ornithine decarboxylase described in this invention may serve as a potential small molecule drug target for therapeutic intervention.

The protein similarity information, expression pattern, cellular localization, and map location for the NOV34 protein and nucleic acid disclosed herein suggest that this Ornithine Decarboxylase-like protein may have important structural and/or physiological functions characteristic of the Ornithine Decarboxylase family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV34 nucleic acids and proteins of the invention have applications in the diagnosis and/or treatment of various diseases and disorders. For example, the compositions of the present invention will have efficacy for the treatment of patients suffering from: hemophilia, hypercoagulation, idiopathic thrombocytopenic purpura, autoimmune disease, allergies, immunodeficiencies, transplantation, graft versus host disease, lymphedema, allergies, fertility, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration as well as other diseases, disorders and conditions.

These antibodies may be generated according to methods known in the art, using prediction from hydrophobicity charts, as described in the “Anti-NOVX Antibodies” section below. The disclosed NOV34 protein has multiple hydrophilic regions, each of which can be used as an immunogen. In one embodiment, a contemplated NOV34 epitope is from about amino acids 7 to 10. In another embodiment, a contemplated NOV34 epitope is from about amino acids 15 to 20. In other specific embodiments, contemplated NOV34 epitopes are from about amino acids 25 to 30, 38 to 42, 55 to 70, 95 to 110, 148 to 150, 160 to 163 and 170 to 190.

NOV35 and NOV36

Two proteins of the invention, referred to herein as NOV35, and NOV36 include Short Chain Dehydrogenase/Reductase-like proteins.

NOV35

A disclosed NOV35 (designated CuraGen Acc. No. CG57339-01), which encodes a novel Short-Chain Dehydrogenase/Reductase-like protein and includes the 2972 nucleotide sequence (SEQ ID NO:107) is shown in Table 35A. An open reading frame for the mature protein was identified beginning with an ATG initiation codon at nucleotides 690-692 and ending with a TGA stop codon at nucleotides 2970-2972. Putative untranslated regions are underlined in Table 35A, and the start and stop codons are in bold letters.

TABLE 35A
NOV35 Nucleotide Sequence

(SEQ ID NO:107)
TTTTTCTTTTTTTTCGAGACGCAGTCTTGCTCTGTCGCCAGGCTGGGGTGCAGTGGCGCAGTCTCTGCTCACT
GCAACCTCCATCTCCCGGGTTCAAGTGACTCTCCTTCCTCAGCCTCCCCACTTCAGTTTCTTTATCTGTCAAT
TGTGGTTAGTGGGCTGTTAATGAAAATTATTAGGTCAAACATCTACTAAGTATCTGTCACATAGTAGGCTCTT
CGTCAATTGGCCCTTTTCCTTCCCACTAGACAACTTGAGAAAGCTTCCTCCTAGCCTATAGCTACTCTTCCGT
TCCACTTCTTGGTTTCCTGCTCTGATTGCCATGTTTTGTTCTCACAGAGGCAGGAGAGGCAGGTCCGAGACCG
CGGGGTGACCCGGTCCAAGGCGGAAAAAGTGCGGCCGCCCACTGTGCCAGTGCCGCAGGTGGATATTGTGCCT
GGGCGGCTCAGTGAGGCCGAGTGGATGGCGCTTACAGCCCTCGAGGAGGGCGAGGACGTCGTAGGGGACATCT
TGGCCGACTTGCTGGCTCGAGTCATGGACTCTGCTTTCAAAGTCTACCTGACTCAGCAGGTGGGCCGGGATCC
GGGTCCTTCAGACTCGTCTCCCTCTCCCGCCCCTCCCTGCCGACCTGAGATCCTCTCTCGCCTCCGCAGTGCA
TTCCATTCACCATCAGCCAGGCCCGGGAGGCCATGCTGCAGATCACCGAGTGGCGCTTCCTGGCCCGGGACGA
GGGAGAATCTGCAGTAGCTGAGGACCCCACATGGGGTGAGGACGAGGAGCCTTCGGCATGCACGACGGACTCC
TGGGCTCAGGGTTCAGTGCCCGTGCTGCACGCGTCCACCTCGGAGGGCCTGGAGAACTTCCAAGGCGAAGTAC
ACTCCTCAGGAGCCTCTCCGGACTCCTCTGCCATTGCTCCTGCTCTCCCCTTTCCGACATCTCACTGCCCGAG
TGCATTTCCCCAGGACCCTGGGGGCGTGGACCGGATCCCTTTAGGAAGGTCGTGGATGGGTCGAGGCTCCCAG
GAGCAGATGGAATCTTGGGAGCCTTCTCCGCAGCTGAGAGTCACGTCGGCCCCTCCTCCCACATCAGAGCTGT
TTCAGGAGGCAGGGCCCGGAGGTCCTGTAGAGGAAGCGGACGGCCAGTCTAGAGGCCTCTCCTCGGCCGGGTC
CTTGAGCGCGAGCTTCCAACTGTCGGTGGAGGAGGCGCCTGCCGACGATGCCGACCCTTCTCTGGATCCGTAC
CTGGTAGCCAGCCCCCAGGCCTCAACTGGGAGGGGACACCCCCTCGGCTTCCATTTGTCGTTGGAAGACCTCT
ACTGTTGCATGCCTCAACTGGACGCGGCTGGGGATCGGCTGGAACTCAGGTCAGAGGGGGTGCCCTGCATCGC
CTCGGGCGTGTTGGTGTCCTACCCCTCTGTGGGCGGCGCCACCCGCCCCTCCGCGTCCTGCCAGCAGCAGCGG
GCCGGGCACTCGGATGTGCGGCTGAGCGCCCACCACCACAGGATGCGCCGCAAGGCGGCCGTGAAACGCCTGG
ACCCTGCGAGGCTCCCGTGCCACTGGGTGCGCCCTCTGGCTGAGGTCCTGGTCCCAGACTCTCAAACACGCCC
CTTGGAAGCCTACCGCGGACGCCAGCGGGGCGAGAAGACCAAGGCCCGGGCCGAACCCCAAGCCCTCGGCCCC
GGCACCCGTGTCTCCCCGGCAGCGTTCTTCCCTCTCCGGCCAGGCATTCCTTTCCGTGACTTGGACTCGGGCC
CCGCACTCCTGTTCCCCACTTTAAATTTAGGCCTATCGTCGCCATCCCTCGAGTCAAAGCTGCCACTCCCAAA
CTCCAGGATCCGCTTCCTCACCACACACCCGGTGCTCCCTGATGTGGCCCGCAGCCGCAGCCCCAAGCTGTGG
CCCAGTGTCAGGTGGCCCAGCGGTTGGGAGGGGAAGGCCGAGCTGCTGGGCGAGCTGTGGGCTGGCCGGACCC
GCGTGCCTCCACAGGGTCTGGAGCTGGCAGACAGGGAGGGCCAGGATCCTGGCAGATGGCCTCGAACCACACC
CCCGGTCCTTGAAGCCACTTCCCAGGTGATGTGGAAGCCCGTGTTGCTGCCAGAAGCCCTGAAGCTGGCCCCT
GGTGTGAGCATGTGGAACCGGAGCACCCAGGTGTTGCTCAGCTCTGGTGTGCCTGAACAAGAGGACAAAGAAG
GTAGCACCTTTCCTCCCGTTGAGCAACATCCCATCCAGACAGGTGCCCCAAAGCCCAGCATTTCCCCAGCAGG
CCCAGGAAGTTTCTGCTATGTTGCTGTGGGCTGCACTCAGCATCCTGGTCTGGGGCGCTGGCTCTGTCTTCCT
TATTCTGGTCTTCTTCAACTACATGTGCAGCTCTGGCAGAAGTCTCATCCCTGGGACCTCCAGTGCTGCTCCA
CAGATCTGACTGGGAAAATAGCCATAGTGACTGGGGCCAACAGTGGCATCGGGAAGGTTGTATCCCAGGACCT
AGCTCGGTGTGGGGCCCAAGTGATCCTTACTTGTCAGAGCAGGGAATGTGGACAGCAAGCCCTGGCTGAGATC
CAAGCAGCCTCAAACAGCAACCGCCTCCTGCTTGGCGAGGTGGACCTTAGCTCCATGACCTCTATTCGGAGCT
TTGCCCGGAGGCTTCTACAGGAGAATCCTGAGATACATCTGCTGGTAAACAATGCTGGAGTCAGTGGATTCCG
AAGACACTTACCCCAGGGGGCCTGGATCTCACCTTTGTCACTAACTATGTTGGGCCCTTTCTGCTCACAAATC
TACTCCAAGGATCTCAAACAAGGTGTACTCCCAGTCCTCTACTTGAGCTTGGCAGAGGAGCCGGGTGGTATTT
CTGGAAAATATTTCAGCAGTTCCTGTGTGATAACTCTTCCCGTTAAAGCCTCTCGGGATCCTCATGTTGCCCA
GAGCCTCTGGAATGCCTCAGTCCGACTGACAAGCCTAGTCAAGATGGACTGA

The disclosed NOV35 nucleic acid sequence maps to chromosome 2 and has 108 of 126 bases (85%) identical to a gb:GENBANK-ID:HUMZB55G05|acc:AF086155.1 mRNA from Homo sapiens (Homo sapiens full length insert cDNA clone ZB55G05) (E=7.4e⁻¹³).

A disclosed NOV35 polypeptide (SEQ ID NO:108) is 760 amino acid residues in length and is presented using the one-letter amino acid code in Table 35B. The SignalP, Psort and/or Hydropathy results predict that NOV35 does not have a signal peptide and is likely to be localized to the mitochondrial matrix space with a certainty of 0.3600. In alternative embodiments, a NOV35 polypeptide is located to the microbody (peroxisome) with a certainty of 0.3051 or the lysosome (lumen) with a certainty of 0.1000.

TABLE 35B
Encoded NOV35 Protein Sequence

(SEQ ID NO:108)

MLQITEWRFLARDEGESAVAEDPTWGEDEEPSACTTDSWAQGSVPVLHASTSEGLENFQGEVHSSGASPDSSAI
APALPFPTSHCPSAFPQDPGGVDRIPLGRSWMGRGSQEQMESWEPSPQLRVTSAPPPTSELFQEAGPGGPVEEA
DGQSRGLSSAGSLSASFQLSVEEAPADDADPSLDPYLVASPQASTGRGHPLGFHLSLEDLYCCMPQLDAAGDRL
ELRSEGVPCIASGVLVSYPSVGGATRPSASCQQQRAGHSDVRLSAHHHRMRRKAAVKRLDPARLPCHWVRPLAE
VLVPDSQTRPLEAYRGRQRGEKTKARAEPQALGPGTRVSPAAFFPLRPGIPFRDLDSGPALLFPTLNLGLSSPS
LESKLPLPNSRIRFLTTHPVLPDVARSRSPKLWPSVRWPSGWEGKAELLGELWAGRTRVPPQGLELADREGQDP
GRWPRTTPPVLEATSQVMWKPVLLPEALKLAPGVSMWNRSTQVLLSSGVPEQEDKEGSTFPPVEQHPIQTGAPK
PSISPAGPGSFCYVAVGCTQHPGLGRWLCLPYSGLLQLHVQLWQKSHPWDLQCCSTDLTGKIAIVTGANSGIGK
VVSQDLARCGAQVILTCQSRECGQQALAEIQAASNSNRLLLGEVDLSSMTSIRSFARRLLQENPEIHLLVNNAG
VSGFRRHLPQGAWISPLSLTMLGPFCSQIYSKDLKQGVLPVLYLSLAEEPGGISGKYFSSSCVITLPVKASRDP
HVAQSLWNASVRLTSLVKMD

The NOV35 amino acid sequence was found to have 45 of 98 amino acid residues (45%) identical to, and 67 of 98 amino acid residues (68%) similar to, the 318 amino acid residue ptnr:SPTREMBL-ACC:Q9NRW0 protein from Homo sapiens (Human) (ANDROGEN-REGULATED SHORT-CHAIN DEHYDROGENASE/REDUCTASE 1) (E=1.5e⁻¹⁹).

NOV35 is expressed in at least the following tissues: B cell germinal, lung, testis, prostate, kidney, germ cells, uterus, blood, lymphocyte, thymus, parathyroid, and heart. Expression information was derived from the tissue sources of the sequences that were included in the derivation of the sequence of NOV35. The sequence is also predicted to be expressed in the above tissues because of the expression pattern of (GENBANK-ID: gb:GENBANK-ID:HUMZB55G05|acc:AF086155.1), a closely related Homo sapiens full length insert cDNA clone ZB55G05 homolog in species Homo sapiens.

Possible small nucleotide polymorphisms (SNPs) found for NOV35 are listed in Table 35C.

TABLE 35C
SNPs

| | Nucleotide | | Amino Acid | |
|---|---|---|---|---|
| Variant | Position | Base Change | Position | Base Change |
| 13377032 | 767 | T > C | NA | NA |
| 13377033 | 2084 | C > G | NA | NA |

NOV35 also has homology to the amino acid sequences shown in the BLASTP data listed in Table 35D.

TABLE 35D
BLAST results for NOV35

| Gene Index/ Identifier | Protein/Organism | Length (aa) | Identity (%) | Positives (%) | Expect |
|---|---|---|---|---|---|
| gi\|12838303\|dbj\| | evidence: NAS˜hypo- | 506 | 242/530 | 295/530 | 2e−88 |
| BAB24157.1\| | thetical | | (45%) | (55%) | |
| (AK005628) | protein-putative | | | | |
| | [Mus musculus] | | | | |
| gi\|5668735\|dbj\|B | UBE-1b [Mus | 300 | 48/111 | 72/111 | 2e−18 |
| AA82657.1\| | musculus] | | (43%) | (64%) | |
| (AB030504) | | | | | |
| gi\|5668733\|dbj\|B | UBE-1a [Mus | 293 | 48/111 | 72/111 | 3e−18 |
| AA82656.1\| | musculus] | | (43%) | (64%) | |
| (AB030503) | | | | | |
| gi\|12835589\|dbj\| | cell line | 316 | 48/111 | 72/111 | 3e−18 |
| BAB23296.1\| | MC/9.IL4 derived | | (43%) | (64%) | |
| (AK004413) | transcript 1-data | | | | |
| | source: MGD, | | | | |
| | source | | | | |
| | key: MGI: 102581, | | | | |
| | evidence: ISS˜ | | | | |
| | putative [Mus | | | | |
| | musculus] | | | | |
| gi\|10947000\|ref\| | cell line | 355 | 48/111 | 72/111 | 6e−18 |
| NP_067532.1\| | MC/9.IL4 derived | | (43%) | (64%) | |
| (NM_021557) | transcript 1; | | | | |
| | hypothetical | | | | |
| | protein M42C60 | | | | |
| | [Mus musculus] | | | | |

The homology of these sequences is shown graphically in the ClustalW analysis shown in Table 35E.

[image]

[image]

[image]

Tables 35F and 35G list the domain description from DOMAIN analysis results against NOV35. This indicates that the NOV35 sequence has properties similar to those of other proteins known to contain these domains.

TABLE 35F
Domain Analysis of NOV35

gnl|Pfam|pfam00106, adh_short, short chain dehydrogenase. This family
contains a wide variety of dehydrogenases.
CD-Length = 249 residues, 51.4% aligned
Score = 84.7 bits (208), Expect = 2e−17

| | | | | |
|---|---|---|---|---|
| NOV35: | 577 | TGKIAIVTGANSGIGKVVSQDLARCGAQVILTCQSRECGQQALAEIQAASNSNRLLLGEV | 636 | (SEQ ID NO:426) |
| | | \|\|\|+\|+\|\|\|\|+\|\|\|\| +++ \|\| \|\|+\|++ + \| +\| \|\|++\| +\| \| ++ | | |
| Sbjct: | 1 | TGKVALVTGASSGIGLAIAKRLAEEGAKVVVVDRREE-KAEAAAELKAE-LGDRALFIQL | 58 | (SEQ ID NO:427) |
| NOV35: | 637 | DLSSMTSIRSFARRLLQENPEIHLLVNNAGVSGFRR--HLPQGAWISPLSLTMLGPF-CS | 693 | |
| | | \|++ \|\|++ + ++\| + +\|\|\|\|\|\|+ \| \| + \| + + + \| \| \| | | |
| Sbjct: | 59 | DVTDEESIKAAVAQAVEELGRLDVLVNNAGILGPGEPFELSEDDWERVIDVNLTGVFLLT | | |
| 118 | | | | |
| NOV35: | 694 | QIYSKDLKQG 703 | | |
| | | \| + + | | |
| Sbjct: | 119 | QAVLPHMLKR 128 | | |

TABLE 35G
Domain Analysis of NOV35

Scores for sequence family classification
(score includes all domains):

| Model | Description | Score | E-value | N |
|---|---|---|---|---|
| adh short (InterPro) | short chain dehydrogenase | 130.8 | 2.6e−35 | 1 |
| SpoU methylase (InterPro) | SpoU rRNA Methylase family | 24.6 | 2.8e−06 | 1 |
| ldh (InterPro) | lactate/malate dehydrogenase | 5.3 | 1.3 | 1 |
| P2X receptor (InterPro) | ATP P2X receptor | 4.1 | 3.8 | 1 |

Parsed for domains:

| Model | Domain | seq-f | seq-t | hmm-f | hmm-t | score | E-value |
|---|---|---|---|---|---|---|---|
| ldh | 1/1 | 10 | 31 . . . | 1 | 25 [. | 5.3 | 1.3 |
| P2X_receptor | 1/1 | 159 | 181 . . . | 372 | 395 .] | 4.1 | 3.8 |
| adh_short | 1/1 | 7 | 189 . . . | 1 | 206 [] | 130.8 | 2.6e−35 |
| SpoU_methylase | 1/1 | 490 | 607 . . . | 1 | 152 [] | 24.6 | 2.8e−06 |

NOV36

A disclosed NOV36 (designated CuraGen Acc. No. CG57341-01), which encodes a novel Short-Chain Dehydrogenase/Reductase-like protein and includes the 2077 nucleotide sequence (SEQ ID NO:109) is shown in Table 36A. An open reading frame for the mature protein was identified beginning with an ATG initiation codon at nucleotides 1-3 and ending with a TGA stop codon at nucleotides 1978-1980. Putative untranslated regions are underlined in Table 36A, and the start and stop codons are in bold letters.

TABLE 36A
NOV36 Nucleotide Sequence

(SEQ ID NO:109)

ATGGAGCGGTGGCGCGACCGGCTGGCGCTGGTGACGGGGGCCTCGGGGGGCATCGGCGCGGCCGTGGCCCGGGC
CCTGGTCCAGCAGGGACTGAAGGTGGTGGGCTGCGCCCGCACTGTGGGCAACATCGAGGAGCTGGCTGCTGAAT
CGTAAGAGTGCAGGCTACCCCGGGACTTTGATCCCCTACAGATGTGACCTATCAAATGAAGAGGACATCCTCTCC
CATGTTCTCAGCTATCCGTTCTCAGCACAGCGGTGTAGACATCTGCATCAACAATGCTGGCTTGGCCCGGCCTGA
CCACCCTGCTCTCAGGCAGCACCAGTGGTTGGAAGGACATGTTCAATGTGAACGTGCTGGCCCTCAGCATCTGCA
CCACGGGAAGCCTACCAGTCCATGAAGGAGCGGAATGTGGACGATGGGCACATCATTAACATCAATAGCATGTCT
CGGCCACCGAGTGTTACCCCTGTCTGTGACCCACTTCTATAGTGCCACCAAGTATGCCGTCACTGCGCTGACAGA
CGGGACTGAGGCAAGAGCTTCGGGAGGCCCAGACCCACATCCGAGCCACGTGGCAGCTTCGGAGGGAGGAGGCCG
CCTGCCGGATATCAGGCAGCCATCACTGTGAAGCTGGGGTTCTGTGGCCTCCATCCTCTCCCCTCGACCTCCCCA
CAGACCTGGCAAAGCTCAGCCCCTGAGAAGGCCCTCTCTGTTGGCCCAGTGCATCTCTCCAGGTGTGGTGGAGAC
CACAATTCGCCTTCAAACTCCACGACAAGGACCCTGAGAAGGCAGCTGCCACCTATGAGCAAATGAAGTGTCTCA
CAACCCGAGGATGTGGCCGAGGCTGTTATCTACGTCCTCAGCACCCCCGCACACATCCAGATTGGAGACATCCAG
CATGAGGCCCACGGAGCAGAGAGCTCGGCGGAGACGGCTGTCGAGTACCCTTCACCTCGGTGTTGGGAGCCTGGG
CAGCGAACTGCGGCGCGGGTTACCGCTCCCGGGGACGCAGCAAGGGGCATCGAGTCCCTGGCGGGAGCTGCGCCA
CTGGCATTGCTCTCGACCGTCCGGGGCGCGACCTGGGGTCGCCTCGTCACCCGTCATTTCTCCCATGCAGCGCGG
CCATGGGGAGCGGCCTGGTGGGGAGGAGCTAAGCCGCTTGCTGCTGGATGACCTGGTGCCGACCTCTCGGCTGGA
CGCTTCTGTTTGGCATGACCCCGTGTCTCCTGGCTCTGCAGGCCGCCCGCCGCTCTGTGGCCCGGCTCCTGCTCC
CAGGCGGGTAAAGCTGGGCTGCAGGGGAAGCGGGCCGAGCTGCTCCGGATGGCCGAGGCGCGGGACATTCCAGTT
CCTGCGGCCCAGACGGCAGAAACTGGACACAATGTGCCGCTACCAGGTCCACCAGGGTGTCTGCATGGAGGTGAG
CCCCGCTGCGGCCCCGGCCTTGGAGAGAGGCCGGGGAGGCGAGCCCAGGCGACGACCCCCAGCAGTTGTGGCTCG
CTCCTCGATGGGATCCAGGATCCCCGGAATTTTGGGGCTGTGCTGCGTTCCGCACACTTCCTCGGAGTGGATAAG
CACCAAAGCCCAGCAGGGCTGGCTCGTGGCCGGCACGGTGGGCTGCCCAAGCACAGAGGATCCCCAGTCCTCCGA
CGATCCCCATCATGAGTTGCTTGGAGTTCCTCTGGGAACGGCCTACTCTCCTTGTGCTGGGGAATGAGGGCTCAG
CGTCTATCCCAGGAGGTGCAGGCCTCCTGCCAGCTTCTCCTCACCATCCTGCCCCGGCGCCAGCTGCCTCCTGGA
CCTTGAGTCCTTGAACGTCTCTGTGGCTGCAGGAATTCTTCTTCACTCCATTTGCAGCCAGAGGAAGGGTTTCCC
CCACAGAGGGGGAGAGAAGGCAGCTTCTCCAAGACCCCCAAGAACCCTCAGCCAGGTCTGAAGGGCTCAGCATGG
CCTCAGCACCCAGGGCTGTCTTCAGGCCCAGAGAAAGAGAGGCAAAATGAGGGCTGACGTGGACTGTCCACAGTG
CTTCATGTGCTGGAGTCAGGGACGGCCGCACCTGCCTCCGCCGGCTCCAGTGTGCGGGGAGCCTCTGCCTGAGTG
CTGCAC

The disclosed NOV36 nucleic acid sequence maps to chromosome 17 and has 261 of 437 bases (59%) identical to a gb:GENBANK-ID:AB035548|acc:AB035548.1 mRNA from Streptomyces virginiae (Streptomyces virginiae orf4, orf5 genes for ketoacyl ACP/CoA reductase homolog, dNDP-glucose dehydratase homolog, complete and partial cds) (E=7.0e⁻⁰⁵).

A disclosed NOV36 polypeptide (SEQ ID NO:110) is 659 amino acid residues in length and is presented using the one-letter amino acid code in Table 36B. The SignalP, Psort and/or Hydropathy results predict that NOV36 does not have a signal peptide and is likely to be localized to the mitochondrial intermembrane space with a certainty of 0.7500. In alternative embodiments, a NOV36 polypeptide is located to the nucleus with a certainty of 0.6000, the mitochondrial matrix space with a certainty of 0.3600 or the microbody (peroxisome) with a certainty of 0.3000.

TABLE 36B
Encoded NOV36 Protein Sequence

(SEQ ID NO:110)

MERWRDRLALVTGASGGIGAAVARALVQQGLKVVGCARTVGNIEELAAECKSAGYPGTLIPYRCDLSNEEDILSM
FSAIRSQHSGVDICINNAGLARPDTLLSGSTSGWKDMFNVNVLALSICTREAYQSMKERNVDDGHIININSMSGH
RVLPLSVTHFYSATKYAVTALTEGLRQELREAQTHIRATWQLRREEAAAGYQAAITVKLGFCGLHPLPSTSPRPG
KAQPLRRPSLLAQCISPGVVETQFAFKLHDKDPEKAAATYEQMKCLKPEDVAEAVIYVLSTPAHIQIGDIQMRPT
EQRARRRRLSSTLHLGVGSLGANCGAGYRSRGRSKGHRVPGGSCAMALLSTVRGATWGRLVTRHFSHAARHGERP
GGEELSRLLLDDLVPTSRLELLFGMTPCLLALQAARRSVARLLLQAGKAGLQGKRAELLRMAEARDIPVLRPRRQ
KLDTMCRYQVHQGVCMEVSPLRPRPWREAGEASPGDDPQQLWLVLDGIQDPRNFGAVLRSAHFLGVDKTKAQQGW
LVAGTVGCPSTEDPQSSEIPIMSCLEFLWERPTLLVLGNEGSGLSQEVQASCQLLLTILPRRQLPPGLESLNVSV
AAGILLHSICSQRKGFPTEGERRQLLQDPQEPSARSEGLSMAQHPGLSSGPEKERQNEG

The NOV36 amino acid sequence was found to have 74 of 192 amino acid residues (38%) identical to, and 114 of 192 amino acid residues (59%) similar to, the 251 amino acid residue ptnr:SPTREMBL-ACC:Q9XYN2 protein from Drosophila melanogaster (Fruit fly) (ANTENNAL-SPECIFIC SHORT-CHAIN DEHYDROGENASE/REDUCTASE) (E=1.7e⁻³⁷).

NOV36 is predicted expressed in at least the following tissues: lung, corresponding non cancerous liver tissue, colon, heart, uterus, skin, brain, and placenta. Expression information was derived from the tissue sources of the sequences that were included in the derivation of the sequence of NOV36. The sequence is predicted to be expressed in the above tissues also because of the expression pattern of (GENBANK-ID: gb:GENBANK-ID:AB035548|acc:AB035548.1) a closely related Streptomyces virginiae orf4, orf5 genes for ketoacyl ACP/CoA reductase homolog, dNDP-glucose dehydratase homolog, complete and partial cds homolog in species Streptomyces virginiae.

NOV36 also has homology to the amino acid sequences shown in the BLASTP data listed in Table 36C.

TABLE 36C
BLAST results for NOV36

| Gene Index/ | | | | | |
|---|---|---|---|---|---|
| Identifier | Protein/Organism | Length (aa) | Identity (%) | Positives (%) | Expect |
| gi\|13236542\|ref\| | hypothetical | 181 | 179/228 | 179/228 | 4e−95 |
| NP_077284.1\| | protein MGC4172 | | (78%) | (78%) | |
| (NM_024308) | [Homo sapiens] | | | | |
| gi\|104389S8\|dbj\| | unnamed protein | 181 | 178/228 | 178/228 | 2e−93 |
| BAB15390.1\| | product [Homo | | (78%) | (78%) | |
| (AK026196) | sapiens] | | | | |
| gi\|14495621\|gb\|A | hypothetical | 158 | 142/145 | 143/145 | 8e−73 |
| AH09416.1\|AAH094 | protein FLJ22578 | | (97%) | (97%) | |
| 16 (BC009416) | [Homo sapiens] | | | | |
| gi\|13376296\|ref\| | hypothetical | 155 | 142/145 | 143/145 | 8e−73 |
| NP_079140.1\| | protein FLJ22578 | | (97%) | (97%) | |
| (NM_024864) | [Homo sapiens] | | | | |
| gi\|13542856\|gb\|A | Similar to | 124 | 91/114 | 99/114 | 1e−43 |
| AH05625.1\|AAH056 | hypothetical | | (79%) | (86%) | |
| 25 (BC005625) | protein FLJ22578 | | | | |
| | [Mus musculus] | | | | |

The homology of these sequences is shown graphically in the ClustalW analysis shown in Table 36D.

[image]

[image]

[image]

Table 36E lists the domain description from DOMAIN analysis results against NOV35. This indicates that the NOV35 sequence has properties similar to those of other proteins known to contain these domains.

TABLE 36E
Domain Analysis of NOV36

gnl|Pfam|pfam00106, adH_short, short chain dehydrogenase. This family contains
a wide variety of dehydrogenases.
CD-Length = 249 residues, 69.9% aligned
Score = 144 bits (364), Expect = 1e-35

| | | | | |
|---|---|---|---|---|
| NOV36: | 6 | DRLALVTGASGGIGAAVARALVQQGLKVVGCARTVGNIEELAAECKSAGYPGTLIPYRCD | 65 | (SEQ ID NO:433) |
| | | ++\|\|\|\|\|\|\| \|\|\| \|+\|+ \| ++\| \|\|\| \| \| \| \| + + \| | | |
| Sbjct: | 2 | GKVALVTGASSGIGLAIAKRLAEEGAKVVVVDRREEKAEAAAELKAELG--DRALFIQLD | 59 | (SEQ ID NO:434) |
| NOV36: | 66 | LSNEEDILSMFSAIRSQHSGVDICINNAGLARPDTLLSGSTSGWKDMFNVNVLALSICTR | 125 | |
| | | +++\|\| \| + + + +\|+ +\|\|\|\|+ \| \| \|+ + +\|\|+ + + \|+ | | |
| Sbjct: | 60 | VTDEESIKAAVAQAVEELGRLDVLVNNAGILGPGEPFELSEDDWERVIDVNLTGVFLLTQ | 119 | |
| NOV36: | 126 | EAYQSMKERNVDDGHIININSMSGHRVLPLSVTHFYSATKYAVTALTEGLRQELREAQTH | 185 | |
| | | \| +\|+ \| \|+\|\|+\|++\| \| \|\|\|+\| \|\| \| \| \|\| \| | | |
| Sbjct: | 120 | AVLPHMLKRS--GGRIVNISSVAGLVPSPGLSA--YSASKAAVVGFTRSLALEL--APHG | 173 | |
| NOV36: | 186 | IR 187 | | |
| | | \|\| | | |
| Sbjct: | 174 | IR 175 | | |

gnl|Pfam|pfam00588, SpoU_methylase, SpoU rRNA Methylase family. This family of
proteins probably use S-AdoMet.
CD-Length = 143 residues, 97.9% aligned
Score = 60.8 bits (146), Expect = 2e-10

| | | | | |
|---|---|---|---|---|
| NOV36: | 493 | LVLDGIQDPRNFGAVLRSAHFLGVD----------------------------------- | 517 | (SEQ ID NO:435) |
| | | +\|\|\| ++ \| \| \|\|++\|+ \|\|\|\| | | |
| Sbjct: | 4 | VVLDEVEIPHNIGAIIRTCAALGVDGIVIVDDGFALLDRRLRRASLGYAESVPVIRVDNL | 63 | (SEQ ID NO:436) |
| NOV36: | 518 | -----KTKAQQGWLVAGTVGCPSTEDPQSSEIPIMSCLEFLWERPTLLVLGNEGSGLSQE | 572 | |
| | | \| \|\|+ ++ \| + + + \|\| \|+\| +\|\|\| | | |
| Sbjct: | 64 | EEFLAHLKESGIWLLT------TSGDGNADPLD-----YEDGAKRLALVFGSETTGLSNL | 112 | |
| NOV36: | 573 | VQASCQLLLTILPRRQLPPGLESLNVSVAAGILLH | 607 | |
| | | + \| + + \|\|\|\|\|\|\| \|+\|\|+ | | |
| Sbjct: | 113 | ALEPADQRIRI----PMNGDVRSLNVSVAVGLLLY | 143 | |

gnl|Pfam|pfam01370, Epimerase, NAD dependent epimerase/dehydratase family.
This family of proteins utilise NAD as a cofactor. The proteins in this family
use nucleotide-sugar substrates for a variety of chemical reactions.
CD-Length = 310 residues, 49.0% aligned
Score = 37.7 bits (86), Expect = 0.002

| | | | | |
|---|---|---|---|---|
| NOV36: | 10 | LVTGASGGIGAAVARALVQQG-LKVVGCAR--TVGNIEELAAECKSAGYPGTLIPYRCDL | 66 | (SEQ ID NO:437) |
| | | \|\|\|\| +\| \|\|+ + \| \|+ \| \|\|\| \|\| \| \| + \|+ | | |
| Sbjct: | 2 | LVTGGAGFIGSHLVRELLNNGDDKVVVLDNLTYAGNEARLRVIEGGPRY----TFVKGDI | 57 | (SEQ ID NO:438) |
| NOV36: | 67 | SNEEDILSMFSAIRSQHSGVDICINNAGLARPDTLLSGSTSGWKDMFNVNVLALSICTRE | 126 | |
| | | + + + +\| + \| \|+ \| + \| + + \|\| \| \|++ | | |
| Sbjct: | 58 | CDRDLLDKVF-----AENQPDAVIHFAAESHVDRSIEKPLAYIDT--NV-VGTLTLL--- | 106 | |
| NOV36: | 127 | AYQSMKERNVDDGHIININSMSG-HRVLPLSVTH------FYSATKYAV 168 | | |
| | | ++ ++ \| + + + \| +\|++ \| \|+\| + | | |
| Sbjct: | 107 | --EAARKAGVFKFVFSSTDEVYGDLPSIPITEDTPYGPSSPYGASKASS 153 | | |

The novel human short chain dehydrogenase/reductase-like proteins of the invention contains dehydrogenase/reductase domains. Therefore it is anticipated that these novel proteins have a role in the regulation of essentially all cellular functions and could be potentially important targets for drugs. Such drugs may have important therapeutic applications.

The short-chain dehydrogenases/reductases family (SDR) (See Joermvall et al., Biochemistry 34: 6003-6013 (1995); InterPro IPR002198) is a very large family of enzymes, most of which are known to be NAD- or NADP-dependent oxidoreductases. As the first member of this family to be characterized was Drosophila alcohol dehydrogenase, this family used to be called ‘insect-type’, or ‘short-chain’ alcohol dehydrogenases. Most member of this family are proteins of about 250 to 300 amino acid residues. Most dehydrogenases possess at least 2 domains, the first binding the coenzyme, often NAD, and the second binding the substrate. This latter domain determines the substrate specificity and contains amino acids involved in catalysis. Little sequence similarity has been found in the coenzyme binding domain although there is a large degree of structural similarity, and it has therefore been suggested that the structure of dehydrogenases has arisen through gene fusion of a common ancestral coenzyme nucleotide sequence with various substrate specific domains. This indicates that the sequence of the invention has properties similar to those of other proteins known to contain this/these domain(s) and similar to the properties of these domains.

Wang et al. (J Biol Chem Apr. 9, 1999;274(15):10309-15) show that a short chain dehydrogenase/reductase and a cytochrome P450 are expressed specifically or preferentially in the olfactory organs, the antennae. The evolutionarily conserved expression of biotransformation enzymes in olfactory organs suggests that they play an important role in olfaction.

The protein similarity information, expression pattern, cellular localization, and map location for the NOV35 and NOV36 proteins and nucleic acids disclosed herein suggest that this short-chain dehydrogenase/reductase-like protein may have important structural and/or physiological functions characteristic of the dehydrogenase/reductase family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV35 and NOV36 nucleic acids and proteins of the invention have applications in the diagnosis and/or treatment of various diseases and disorders. For example, the compositions of the present invention will have efficacy for the treatment of patients suffering from: systemic lupus erythematosus, autoimmune disease, asthma, emphysema, scleroderma, allergy, ARDS, Von Hippel-Lindau (VHL) syndrome, cirrhosis, transplantation, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderrna, obesity, transplantation, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, psoriasis, actinic keratosis, tuberous sclerosis, acne, hair growth/loss, allopecia, pigmentation disorders, endocrine disorders, endometriosis, fertility as well as other diseases, disorders and conditions.

These antibodies may be generated according to methods known in the art, using prediction from hydrophobicity charts, as described in the “Anti-NOVX Antibodies” section below. The disclosed NOV35 protein has multiple hydrophilic regions, each of which can be used as an immunogen. In one embodiment, a contemplated NOV35 epitope is from about amino acids 2 to 20. In another embodiment, a contemplated NOV35 epitope is from about amino acids 60 to 65. In other specific embodiments, contemplated NOV35 epitopes are from about amino acids 105 to 130, 160 to 167, 190 to 220, 221 to 225, 270 to 290, 310 to 320, 390 to 410, 425 to 460, 490 to 515, 570 to 580, 610 to 620, 670 to 690, 760 to 770. The disclosed NOV36 protein has multiple hydrophilic regions, each of which can be used as an immunogen. In one embodiment, a contemplated NOV36 epitope is from about amino acids 50 to 70. In another embodiment, a contemplated NOV36 epitope is from about amino acids 100 to 105. In other specific embodiments, contemplated NOV36 epitopes are from about amino acids 110 to 120, 190 to 200, 220 to 225, 270 to 275, 290 to 305, 320 to 325, 380 to 385, 430 to 460, 490 to 505 and 610 to 660.

NOV37

A disclosed NOV37 (designated CuraGen Acc. No. CG57335-01), which encodes a novel Protocadberin beta 3-like protein and includes the 3010 nucleotide sequence (SEQ ID NO:111) is shown in Table 37A. An open reading frame for the mature protein was identified beginning with an ATG initiation codon at nucleotides 429A431 and ending with a TAA stop codon at nucleotides 2817-2819. Putative untranslated regions are underlined in Table 37A, and the start and stop codons are in bold letters.

TABLE 37A
NOV37 Nucleotide Sequence

(SEQ ID NO:111)

AATCTTTTTTTTTTTTTTTTTTTTCGTAGATAAAAGTGCATTTTATTTCCCTAGATTGCATTTATTTAATTCATATAA
CATGAGAAACTCCTCCAGTAGCGTCAACTAGGGTTGATAAGAATAATCGATAAAGCAAAATAAAAACACCTTCTCCAA
GATTTTGTAACTGCAAGCGAACGCATGGTGGCGCTGTTGACTAAGAAGGCGAATTAAACCACAGGCATTGTGCATGCT
CGGTGACGCACGGATCCAGTGTGGTAAACCAGCGGTTGAGAGCCCAGGCAGATTTTTGAGCCAGCAAGTCTGAGCCTC
TGGAAAGGCTTATTCACTAGGCCGTCTACAAAGGTTGTGGGGCAAAAGACTGTTTCCCAGCTCTGTCTGAGGTTCAGC
TTGGCGACATTCCCTGGAAGAGCGTGACGGAAAGTGCAATGGAGGCGGGAGGAGAGCGATTTCTTAGACAAAGGCAAG
TCTTGCTTCTCTTTGTTTTTCTGGGAGGGTCTCTGGCTGGGTCCGAGTCAAGACGCTATTCTGTGGCTGAGGAAAAAG
AGAAGGGCTTTTTAATAGCCAACCTAGCAAAGGATCTGGGACTAAGGGTAGAGGAACTGGCCGCGAGGGGGGCCCAAG
TTGTGTCCAAAGGGAACAAACAGCATTTTCAGCTCAGTCATCAGACAGGTGATTTGCTCCTGAATGAGAAATTGGACC
GGGAGGAGCTATGCGGCCCCACAGAACCATGCATACTACATTTTCAGATATTACTGCAAAACCCTTTGCAATTCGTTA
CAAACGAGCTCCGTATCATAGATGTAAATGACCATTCTCCGGTATTCTTTGAAAATGAAATGCATCTGAAAATCCTAG
AAAGCACTCTGCCAGGAACAGTAATTCCTTTGGGAAATGCTGAGGACTTGGATGTGGGAAGAAACAGCCTCCAAAACT
ACACTATCACTCCGAATTCCCACTTCCACGTACCCACTCGCAGTCGTAGGGACGGAAGGAAGTACCCGGAACTAGTAC
TGAACAGAGCCCTGGATCGCGAGGAGCAGCCTGAGATCAGGTTAACCCTCACAGCGCTAGATGGCGGGAGTCCACCCA
GGTCCGGCACGGCCCTGGTACCGATTGAAGTTGTGGACATCAATGACAACGTCCCAGAGTTTGCAAAGCTGCTCTATG
AGGTGCAGATCCCGGAGGACAGCCCCGTTGGATCCCAGGTTGCCATCGTCTCTGCCAGGGATTTAGACATTGGAACTA
ATGGAGAAATATCTTATGCATTTTCCCAAGCATCTGAAGACATTCGCAAAACGTTTCGATTAAGTGCAAAATCGGGAG
AACTGCTTTTAAGACAGAAACTGGATTTCGAATCCATCCAGACATACACAGTAAATATTCAGGCGACAGATGGTGGGG
GCCTATCCGGAAAGTCTACAGTCATAGTCCAGGTGGTTGATGTCAACGACAACCCACCGGAACTGACCTTGTCTTCAG
TAAACAGCCCTATTCCTGAGAACTCGGGAGAGACTGTACTGGCTGTTTTCAGTGTTTCTGATCTAGACTCTGGAGACA
ACGGAAGAGTGATGTGTTCCATTGAGAACAATCTCCCCTTCTTCCTGAAACCATCTGTAGAGAATTTTTACACCCTAG
TGTCAGAAGGCGCGCTGGACAGAGAGACCAGATCCGAGTACAACATTACCATCACTATCACTGACCTGGGGACACCCA
GGCTGAAAACCAAGTACAACATAACCGTGCTGGTCTCCGACGTCAATGACAACGCCCCCGCCTTCACCCAAATCTCCT
ACACCCTGTTCGTCCGCGAGAACAACAGCCCCGCCCTGCACATCGGCAGTGTCAGCGCCACAGACAGAGACTCAGGCA
CCAACGCCCAGGTAACCTACTCGCTGCTGCCGCCCCAGGACCCGCACCTGCCCCTCTCTTCCCTGGTCTCCATCAACG
CGGACAACGGCCACCTGTTTGCCCTCAGGTCGCTGGACTACGAGGCCCTGCAGGCGTTCGAGTTCCGCGTGGGCGCCA
CAGACCGTGGCTCCCCGGCTTTGAGCAGCGAGGCGCTGGTGCGCGTGCTGGTGCTGGACGCCAACGACAACTCGCCCT
TCGTGCTGTACCCGCTGCAGAACGGCTCCGCGCCCTGCACCGAGCTGGTGCCCCGGGCGGCTGAGCCGGGCTACCTGG
TGACCAAGGTGGTGGCGGTGGACGGCGACTCGGGCCAGAACGCCTGGCTGTCGTACCAGCTGCTCAAGGCCACGGAGC
CCGGGCTGTTCGGCGTGTGGGCGCACAATGGCGAAGTGCGCACCGCCAGGCTGCTGAGGGAGCGCGACGCTGCCAAGC
AGAGGCTGGTGGTGCTGGTCAAGGACAATGGCGAGCCTCCGCGCTCGGCCACCGCCACGCTGCACGTGCTCCTGGTGG
ACGGCTTCTCCCAGCCCTACCTGCTGCTCCCGGAGGCGGCACCGGCCCAGGCCCAGGCCGACTTGCTCACCGTCTACC
TGGTGGTGGCGTTGGCCTCGGTGTCTTCGCTCTTCCTCTTCTCGGTGCTCCTGTTCGTGGCGGTGCGGCTGTGCAGGA
GGAGCAGGGCGGCCTCGGTGGGTCGCTGCTCGGTGCCCGAGGGCCCCTTTCCAGGGCAGATGGTGGACGTGAGCGGCA
CCGGGACCCTGTCCCAGAGCTACCAGTACGAGGTGTGTCTGACTGGAGGCTCCGGGACAAATGAGTTCAAGTTCCTGA
AGCCAATTATCCCCAACTTCGTTGCTCAGGGTGCAGAGAGGGTTAGCGAGGCAAATCCCAGTTTCAGGAAGAGCTTTG
AATTCACTTAAGTGTTAATAAGGATCTACTGAGGCTAGTCTCGTTTAATTTGTGGAAAGTCCTTTTTTACTGCTTTGC
CCATTGGAGGTGTCTCCTTTTATTAGAAAGTAACCATCTTATTCCAATTCTATGCATGTTACTGGTATTTATAAATGT
ATGAGTTTTTTTGCGGTATAATAAATGTAAATTTTCTTTGTATTCT

The disclosed NOV37 nucleic acid sequence maps to chromosome 5 and has 2257 of 2391 bases (94%) identical to a gb:GENBANK-ID:AF152496|acc:AF152496.1 mRNA from Homo sapiens (Homo sapiens protocadherin beta 3 (PCDH-beta3) mRNA, complete cds) (E=0.0).

A disclosed NOV37 polypeptide (SEQ ID NO:112) is 796 amino acid residues in length and is presented using the one-letter amino acid code in Table 37B. The SignalP, Psort and/or Hydropathy results predict that NOV37 has a signal peptide and is likely to be localized to the plasma membrane with a certainty of 0.4600. In alternative embodiments, a NOV37 polypeptide is located to the endoplasmic reticulum (membrane) with a certainty of 0.1000, the endoplasmic reticulum (lumen) with a certainty of 0.1000 or the outside of the cell with a certainty of 0.1000. The Signap predicts a likely cleavage site for a NOV37 peptide between amino acid positions 26 and 27, i.e. at the sequence SLA-GS.

TABLE 37B
Encoded NOV37 Protein Sequence

(SEQ ID NO:112)

MEAGGERFLRQRQVLLLFVFLGGSLAGSESRRYSVAEEKEKGFLIANLAKDLGLRVEELAARGAQVVSKGNKQHFQL
SHQTGDLLLNEKLDREELCGPTEPCILHFQILLQNPLQFVTNELRIIDVNDHSPVFFENEMHLKILESTLPGTVIPL
GNAEDLDVGRNSLQNYTITPNSHFHVPTRSRRDGRKYPELVLNRALDREEQPEIRLTLTALDGGSPPRSGTALVRIE
VVDINDNVPEFAKLLYEVQIPEDSPVGSQVAIVSARDLDIGTNGEISYAFSQASEDIRKTFRLSAKSGELLLRQKLD
FESIQTYTVNIQATDGGGLSGKSTVIVQVVDVNDNPPELTLSSVNSPIPENSGETVLAVFSVSDLDSGDNGRVMCSI
ENNLPFFLKPSVENFYTLVSEGALDRETRSEYNITITITDLGTPRLKTKYNITVLVSDVNDNAPAFTQISYTLFVRE
NNSPALHIGSVSATDRDSGTNAQVTYSLLPPQDPHLPLSSLVSINADNGHLFALRSLDYEALQAFEFRVGATDRGSP
ALSSEALVRVLVLDANDNSPFVLYPLQNGSAPCTELVPRAAEPGYLVTKVVAVDGDSGQNAWLSYQLLKATEPGLFG
VWAHNGEVRTARLLRERDAAKQRLVVLVKDNGEPPRSATATLHVLLVDGFSQPYLLLPEAAPAQAQADLLTVYLVVA
LASVSSLFLFSVLLFVAVRLCRRSRAASVGRCSVPEGPFPGQMVDVSGTGTLSQSYQYEVCLTGGSGTNEFKFLKPI
IPNFVAQGAERVSEANPSFRKSFEFT

The NOV37 amino acid sequence was found to have 742 of 796 amino acid residues (93%) identical to, and 767 of 796 amino acid residues (96%) similar to, the 796 amino acid residue ptnr:SPTPEMBL-ACC:Q9Y5E6 protein from Homo sapiens (Human) (PROTOCADHERIN BETA 3) (E=0.0).

NOV37 is predicted expressed in at least the following tissues: brain, spinal cord, cartilage, heart, stomach, testis, urinary bladder, and oviduct/uterine tube/fallopian tube. Expression information was derived from the tissue sources of the sequences that were included in the derivation of the sequence of NOV37.

Possible small nucleotide polymorphisms (SNPs) found for NOV37 are listed in Table 37C.

TABLE 37C
SNPs

| | Nucleotide | | Amino Acid | |
|---|---|---|---|---|
| Variant | Position | Base Change | Position | Base Change |
| 13377034 | 302 | G > C | NA | NA |
| 13377035 | 457 | G > T | 10 | Arg > Ile |
| 13377036 | 2537 | C > T | NA | NA |

NOV37 also has homology to the amino acid sequences shown in the BLASTP data listed in Table 37D.

TABLE 37D
BLAST results for NOV37

| Gene Index/ | | Length | Identity | Positives | |
|---|---|---|---|---|---|
| Identifier | Protein/Organism | (aa) | (%) | (%) | Expect |
| gi\|9256614\|ref\|N | protocadherin | 796 | 742/796 | 767/796 | 0.0 |
| P_061760.1\| | beta 3 precursor | | (93%) | (96%) | |
| (NM_018937) | [Homo sapiens] | | | | |
| gi\|9256612\|ref\|N | protocadherin beta | 798 | 693/798 | 730/798 | 0.0 |
| P_061759.1\| | 2 precursor [Homo | | (86%) | (90%) | |
| (NM_018936) | sapiens] | | | | |
| gi\|13431369\|sp\|Q | Protocadherin beta | 776 | 605/774 | 682/774 | 0.0 |
| 9NRJ7\|CDBG_HUMAN | 16 precursor | | (78%) | (87%) | |
| | (PCDH-beta16) | | | | |
| | (Protocadherin 3X) | | | | |
| gi\|14195605\|ref\| | protocadherin | 776 | 603/774 | 681/774 | 0.0 |
| NP_066008.1\| | beta 16 | | (77%) | (87%) | |
| (NM_020957) | precursor; | | | | |
| | protocadherin | | | | |
| | beta 8a; | | | | |
| | protocadherin-3x; | | | | |
| | cadherin ME1 | | | | |
| | [Homo sapiens] | | | | |
| gi\|10047319\|dbj\| | KIAA1621 protein | 787 | 599/774 | 677/774 | 0.0 |
| BAB13447.1\| | [Homo sapiens] | | (77%) | (87%) | |
| (AB046841) | | | | | |

The homology of these sequences is shown graphically in the ClustalW analysis shown in Table 37E.

[image]

[image]

[image]

Table 37F lists the domain description from DOMAIN analysis results against NOV37. Many regions of NOV37 show homology to the cadherin repeats and cadherin domain. This indicates that the NOV37 sequence has properties similar to those of other proteins known to contain these domains.

TABLE 37F
Domain Analysis of NOV37

gnl|Smart|smart00112, CA, Cadherin repeats.; Cadherins are glycoproteins
involved in Ca2+-mediated cell-cell adhesion. Cadherin domains occur as
repeats in the extracellular regions which are thought to mediate cell-cell
contact when bound to calcium.
CD-Length = 82 residues, 100.0% aligned
Score = 85.9 bits (211), Expect = 8e-18

| | | | | |
|---|---|---|---|---|
| NOV37: | 473 | VSATDRDSGTNAQVTYSLLPPQDPHLPLSSLVSINADNGHLFALRSLDYEALQAFEFRVG | 532 | (SEQ ID NO:444) |
| | | \|\|\|\|\| \|\|\| \| +\|\|\|\|+\| \| \| \|\|+ + \| + + \|\| \| + \| | | |
| Sbjct: | 1 | VSATDADSGENGKVTYSILSGNDGGL-----FSIDPETGIITTTKPLDREEQSEYTLTVE | 55 | (SEQ ID NO:445) |
| NOV37: | 533 | ATDRGSPALSSEALVRVLVLDANDNSP 559 | | |
| | | \|\|\| \| \| \|\|\| \| \| \| \|\|\| \|\|\|+\| | | |
| Sbjct: | 56 | ATDGGGPPLSSTATVTVTVLDVNDNAP 82 | | |

gnl|Smart|smart00112, CA, Cadherin repeats.
CD-Length = 82 residues, 100.0% aligned
Score 81.6 bits (200), Expect = 2e-16

| | | | | |
|---|---|---|---|---|
| NOV37: | 264 | VSARDLDIGTNGEISYAFSQASEDIRKTFRLSAESGELLLRQKLDFESIQTYTVNIQATD | 323 | (SEQ ID NO:446) |
| | | \|\|\| \| \| \| \|\|+++\|+ + \| + ++\| + + \|\| \| \|\|+ ++ | | |
| Sbjct: | 1 | VSATDADSGENGKVTYSILS--GNDGGLFSIDPETGIITTTKPLDREEQSEYTLTVEATD | 58 | (SEQ ID NO:445) |
| NOV37: | 324 | GGG--LSGKSTVIVQVVDVNDNPP 345 | | |
| | | \|\|\| \|\| +\|\| \| \|+\|\|\|\|\| \| | | |
| Sbjct: | 59 | GGGPPLSSTATVTVTVLDVNDNAP 82 | | |

gnl|Smart|smart00112, CA, Cadherin repeats.
CD-Length = 82 residues, 98.8% aligned
Score = 79.0 bits (193), Expect = 1e-15

| | | | | |
|---|---|---|---|---|
| NOV37: | 156 | NAEDLDVGRNSLQNYTITPNSHFHVPTRSRRDGRKYPELVLNRALDREEQPEIRLTLTAL | 215 | (SEQ ID NO:447) |
| | | +\| \| \| \| \| \|+\| + + + \| + + \|\|\|\|\|\| \| \|\|+ \| | | |
| Sbjct: | 2 | SATDADSGENGKVTYSILSGNDGGLFSIDPETG----IITTTKPLDREEQSEYTLTVEAT | 57 | (SEQ ID NO:448) |
| NOV37: | 216 | DGGSPPRSGTALVRIEVVDINDNVP 240 | | |
| | | \|\|\| \|\| \| \|\| \| + \|+\|+\|\|\| \| | | |
| Sbjct: | 58 | DGGGPPLSSTATVTVTVLDVNDNAP 82 | | |

gnl|Smart|smart00112, CA, Cadherin repeats
CD-Length = 82 residues, 98.8% aligned
Score = 68.9 bits (167), Expect = 1e-12

| | | | | |
|---|---|---|---|---|
| NOV37: | 369 | SVSDLDSGDNGRVNCSIENNLPFFLKPSVENFYTLVSEGALDRETRSEYNITITITDLGT | 428 | (SEQ ID NO:449) |
| | | \| +\| \|\|\|+\|\|+\| \|\| + \| + + \|\|\|\| +\|\|\| +\|+ \|\| \| | | |
| Sbjct: | 2 | SATDADSGENGKVTYSILSGNDGGLFSIDPETGIITTTEPLDREEQSEYTLTVEATDGGG | 61 | (SEQ ID NO:448) |
| NOV37: | 429 | PRLKTKYNITVLVSDVNDNAP 449 | | |
| | | \| \| + +\|\| \| \|\|\|\|\|\|\| | | |
| Sbjct: | 62 | PPLSSTATVTVTVLDVNDNAP 82 | | |

gnl|Smart|smart00112, CA, Cadherin repeats.
CD-Length = 82 residues, 92.7% aligned
Score = 65.1 bits (157), Expect = 1e-11

| | | | | |
|---|---|---|---|---|
| NOV37: | 589 | VVAVDGDSGQNAWLSYQLLKATEPGLFGVWAHNGEVRTARLLRERDAAKQRLVVLVKDNG | 648 | (SEQ ID NO:450) |
| | | \| \| \| \|\|\|+\| ++\| +\| + \|\|\| + \| + \| + \| + ++ \| \| \| \| | | |
| Sbjct: | 1 | VSATDADSGENGKVTYSILSGNDGGLFSIDPETGIITTTKPLDREEQSEYTLTVEATDGG | 60 | (SEQ ID NO:451) |
| NOV37: | 649 | EPPRSATATLEVLLVD 664 | | |
| | | \|\| \|+\|\|\|+ \| ++\| | | |
| Sbjct: | 61 | GPPLSSTATVTVTVLD 76 | | |

gnl|Pfam|pfam00028, cadherin, Cadherin domain.
CD-Length = 92 residues, 98.9% aligned
Score = 79.3 bits (194), Expect = 8e-16

| | | | | |
|---|---|---|---|---|
| NOV37: | 247 | YEVQIPEDSPVGSQVAIVSARDLDIGTNGEISYAFSQASEDIRKTFRLSAKSGELLLRQK | 306 | (SEQ ID NO:452) |
| | | \| +\|\|++\|\|\|++\| \|+\| \| \|+\| \|\| \| \|+ \|\|+ +\|+\| + | | |
| Sbjct: | 1 | YSASVPENAPVGTEVLTVTATDADLGPNGRIFYSILGGGPG--GWFRIDPDTGDLSTTKP | 58 | (SEQ ID NO:453) |
| NOV37: | 307 | LDFESIQTYTVNIQATDGG--GLSGKSTVIVQV 337 | | |
| | | \|\| \|\|\| \| + + \|\|\| \| \|\|\| +\|\| + \| | | |
| Sbjct: | 59 | LDRESIGEYELTVLATDSGGPPLSGTTTVTITV 91 | | |

gnh|Pfam|pfam00028, cadherin, Cadherin domain.
CD-Length = 92 residues, 100.0% aligned
Score = 76.3 bits (186), Expect = 6e-15

| | | | | |
|---|---|---|---|---|
| NOV37: | 456 | YTLFVRENNSPALHIGSVSATDRDSGTNAQVTYSLLPPQDPELPLSSLVSINADNGHLFA | 515 | (SEQ ID NO:454) |
| | | \|+ \| \|\| + +\|+\|\|\| \| \| \| ++ \|\|+\| \|+ \| \| \| | | |
| Sbjct: | 1 | YSASVPENAPVGTEVLTVTATDADLGPNGRIFYSILGGGPGGW-----FRIDPDTGDLST | 55 | (SEQ ID NO:455) |
| NOV37: | 516 | LRSLDYEALQAFEFRVGATDRGSPALSSEALVRVLVL 552 | | |
| | | + \|\| \|++ +\| \| \|\|\| \| \| \|\| \| + \|\| | | |
| Sbjct: | 56 | TKPLDRESIGEYELTVLATDSGGPPLSGTTTVTITVL 92 | | |

gnl|Pfam|pfam00028, cadherin, Cadherin domain.
CD-Length = 92 residues, 92.4% aligned
Score = 60.1 bits (144), Expect = 5e-10

| | | | | |
|---|---|---|---|---|
| NOV37: | 576 | VPRAAEPGYLVTKVVAVDGDSGQNAWLSYQLLKATEPGLFGVWAHNGEVRTARLLRERDA | 635 | (SEQ ID NO:456) |
| | | \|\| \| \| \| \| \| \| \| \| \| + \| +\| \| \| + \|++ \| + \| | | |
| Sbjct: | 5 | VPENAPVGTEVLTVTATDADLGPNGRIFYSILGGGPGGWFRIDPDTGDLSTTKPLDRESI | 64 | (SEQ ID NO:457) |
| NOV37: | 636 | AKQRLVVLVKDNGEPPRSATATLHV 660 | | |
| | | + \| \|\| \|+\| \|\| \| \| \|+ + | | |
| Sbjct: | 65 | GEYELTVLATDSGGPPLSGTTTVTI 89 | | |

gnl|Pfam|pfam00028, cadherin, Cadherin domain.
CD-Length = 92 residues, 94.6% aligned
Score = 58.5 bits (140), Expect = 1e-09

| | | | | |
|---|---|---|---|---|
| NOV37: | 142 | ILESTLPGTVIPLGNAEDLDVGRNSLQNYTITPNSHFHVPTRSRRDGRKYPELVLNRALD | 201 | (SEQ ID NO:458) |
| | | + \|+ \|\| + \| \| \|+\| \| \|+\| \| +\| + \|\| | | |
| Sbjct: | 5 | VPENAPVGTEVLTVTATDADLGPNGRIFYSILGGGPGGWFRIDPDTG----DLSTTKPLD | 60 | (SEQ ID NO:459) |
| NOV37: | 202 | REEQPEIRLTLTALDGGSPPRSGTALVRIEV 232 | | |
| | | \|\| \| \|\|+ \| \| \| \|\| \|\|\| \| \| \| | | |
| Sbjct: | 61 | RESIGEYELTVLATDSGGPPLSGTTTVTITV 91 | | |

gnl|Pfam|pfam00028, cadherin, Cadherin domain.
CD-Length = 92 residues, 94.6% aligned
Score = 48.5 bits (114), Expect = 1e-06

| | | | | |
|---|---|---|---|---|
| NOV37: | 356 | IPENSGE-TVLAVFSVSDLDSGDNGRVMCSIENNLPFFLKPSVENFYTLVSE-------G | 407 | (SEQ ID NO:460) |
| | | +\|\|\|+ \| + + +\| \| \| \|\|\|+ \|\| \| ++ + + | | |
| Sbjct: | 5 | VPENAPVGTEVLTVTATDADLGPNGRIFYSI-------LGGGPGGWFRIDPDTGDLSTTK | 57 | (SEQ ID NO:459) |
| NOV37: | 408 | ALDRETRSEYNITITITDLGTPRLKTKYNITVLV 441 | | |
| | | \|\|\|\|+ \|\| +\|+ \|\| \| \| \| +\|+ \| | | |
| Sbjct: | 58 | PLDRESIGEYELTVLATDSGGPPLSGTTTVTITV 91 | | |

Cadherins play important roles in specific cell-cell adhesion events and also mediate interactions between cells and the extracellular matrix. They are required for morphogenesis, mediation of neural cell-cell interactions, and the regulation of immune cell responses (Nollet et al., (2000) J. Mol. Biol. 299: 551-72; Henricks and Nijkamp (1998) Eur. J. Pharmacol. 344: 1-13). Many diseases have been shown to be associated with dysfunction of or with overexpression of adhesion molecules. For example, improper cadherin levels have been observed in human cancer malignancies and are thought to lead to cancer cell invasion and metastasis (Nollet et al., (2000) J. Mol. Biol. 299: 551-72). It has also been demonstrated that anti-adhesion treatment can lead to diminished infiltration and activation of inflammatory immune cells resulting in decreased tissue injury and malfunction (Henricks and Nijkamp (1998) Fur. J. Pharmacol. 344: 1-13).

The cadherins form a superfamily of calcium-dependent cell-cell adhesion molecules that can be divided into at least six subfamilies, one of which is known as the protocadherin subfamily (Nollet et al., (2000) J. Mol. Biol. 299: 551-72). Wu and Maniatis identified 52 novel cadherin-like genes, including protocadherin beta 3, on human chromosome 5q31 (Wu and Maniatis (1999) Cell 97: 779-790). The gene described in this invention is a homolog of protocadhern beta 3 and is expressed in the brain, suggesting that it may be involved in neural cell interactions and play a role in diseases of the central nervous system. Furthermore, based on observations from the other cadherin family members, the protocadherin beta 3-like gene may also be involved in cancer or immunological disorders, among other diseases. The protocadherin beta 3-like gene maps to human chromosome 5.

The protein similarity information, expression pattern, cellular localization, and map location for the NOV37 protein and nucleic acid disclosed herein suggest that this Protocadherin beta 3-like protein may have important structural and/or physiological functions characteristic of the Cadherin family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The NOV37 nucleic acids and proteins of the invention have applications in the diagnosis and/or treatment of various diseases and disorders. For example, the compositions of the present invention will have efficacy for the treatment of patients suffering from: cancer, trauma, bacterial and viral infections, in vitro and in vivo regeneration, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, arthritis, tendonitis, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, ulcers, fertility, cystitis, incontinence, and endometriosis as well as other diseases, disorders and conditions.

These antibodies may be generated according to methods known in the art, using prediction from hydrophobicity charts, as described in the “Anti-NOVX Antibodies” section below. The disclosed NOV37 protein has multiple hydrophilic regions, each of which can be used as an immunogen. In one embodiment, a contemplated NOV37 epitope is from about amino acids 20 to 30. In another embodiment, a contemplated NOV37 epitope is from about amino acids 80 to 105. In other specific embodiments, contemplated NOV37 epitopes are from about amino acids 110 to 120, 175 to 210, 240 to 245, 280 to 320, 330 to 335, 390 to 395, 400 to 435, 470 to 490, 510 to 530, 575 to 635 and 720 to 790.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (ie., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 11, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 or 111 is one that is sufficiently complementary to the nucleotide sequence shown SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 or 111 that it can hydrogen bond with little or no mismatches to the nucleotide sequence shown SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111; or an anti-sense strand nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111; or of a naturally occurring mutant of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111 due to degeneracy of the genetic code and thus encode the same NOVX proteins as that encoded by the nucleotide sequences shown in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111. In another embodiment, an isolated nucleic acid molecule of the invention has a nucleotide sequence encoding a protein having an amino acid sequence shown in SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112.

In addition to the human NOVX nucleotide sequences shown in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 10.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112. Preferably, the protein encoded by the nucleic acid molecule is at least about 60% homologous to SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112; more preferably at least about 70% homologous SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112; still more preferably at least about 80% homologous to SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112; even more preferably at least about 90% homologous to SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112; and most preferably at least about 95% homologous to SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112.

An isolated nucleic acid molecule encoding an NOVX protein homologous to the protein of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112 can be created by introducing one or more nucleotide substitutions, additions or deletions into the nucleotide sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, such that one or more amino acid substitutions, additions or deletions are introduced into the encoded protein.

Mutations can be introduced into SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112, or antisense nucleic acids complementary to an NOVX nucleic acid sequence of SEQ ID NOS:1, 3, 5, 7, 9, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, Schlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an o-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (ie., SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S1 nucleases (See, Hyrup, et al., 1996.supra); or as probes or primers for DNA sequence and hybridization (See, Hyrup, et al., 1996, supra; Perry-O'Keefe, et al., 1996. sopra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. Biotechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112. The invention also includes a mutant or variant protein any of whose residues may be changed from the corresponding residues shown in SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112. In other embodiments, the NOVX protein is substantially homologous to SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112, and retains the functional activity of the protein of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112, and retains the functional activity of the NOVX proteins of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S1 nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, ie., molecules that contain an antigen binding site that specifically binds (immunoreacts with) an antigen. Such antibodies include, but are not limited to, polyclonal, monoclonal, chimeric, single chain, F_{ab}, F_{ab}, and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al, MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonbergetal. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouselm as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (ie., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites ford ii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g. replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET lid (Studieret al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et a., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (ie., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequencers) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111), but more preferably, is a non-human homologue of a human NOVX gene. For example, a mouse homologue of human NOVX gene of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111 can be used to construct a homologous recombination vector suitable for altering an endogenous NOVX gene in the mouse genome. In one embodiment, the vector is designed such that, upon homologous recombination, the endogenous NOVX gene is functionally disrupted (ie., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected.

See, e.g., Li, et al., 1992. Cell 69: 915. The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by gernmine transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomzyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; id a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natr. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ull.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:1, 3, 5, 7,9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g. the genotype of the individual examined to determine the ability of the individual to respond to a particular agent.) Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (ie., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86:1173-1177); Qo Replicase (see, Lizardi, et al., 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at GfT mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmnacogenomics of the individual permits the selection of effective agents (e.g., drugs) for prophylactic or therapeutic treatments based on a consideration of the individual's genotype. Such pharmnacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully below.

Disease and Disorders

Diseases and disorders that are characterized by increased (relative to a subject not suffering from the disease or disorder) levels or biological activity may be treated with Therapeutics that antagonize (i e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (ie., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g, an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

General Screeening and Diagnostic Methods

Several of the herein disclosed methods relate to comparing the levels of expression of angiopoietin related protein (ARP) nucleic acids or polypetides in a test and reference cell populations. The sequence information disclosed herein, coupled with nucleic acid detection methods known in the art, allow for detection and comparison of the ARP transcripts.

In its various aspects and embodiments, the invention includes providing a test cell population which includes at least one cell that is capable of expressing ARP. By “capable of expressing” is meant that the gene is present in an intact form in the cell and is expressed under particular conditions. Using sequence information provided by the database entries for the ARP sequences, ARP sequences can be detected (if present) and measured using techniques well known to one of ordinary skill in the art. For example, sequences within the sequence database entries corresponding to ARP, or within the sequences disclosed herein, can be used to construct probes for detecting ARP RNA sequences in, e.g., northern blot hybridization analyses or methods which specifically, and, preferably, quantitatively amplify specific nucleic acid sequences. As another example, the sequences can be used to construct primers for specifically amplifying the ARP sequences in, e.g., amplification-based detection methods such as reverse-transcription based polymerase chain reaction. When alterations in gene expression are associated with gene amplification or deletion, sequence comparisons in test and reference populations can be made by comparing relative amounts of the examined DNA sequences in the test and reference cell populations.

For ARP sequences whose polypeptide product is known, expression can be also measured at the protein level, i.e., by measuring the levels of polypeptides encoded by the gene products described herein. Such methods are well known in the art and include, e.g., immunoassays based on antibodies to proteins encoded by the genes.

Expression level of the ARP sequences in the test cell population is then compared to expression levels of the ARP in one or more cells from a reference cell population. Expression of sequences in test and control populations of cells can be compared using any art-recognized method for comparing expression of nucleic acid sequences. For example, expression can be compared using GENECALLING® methods as described in U.S. Pat. No. 5,871,697 and in Shimkets et al., Nat. Biotechnol. 17:798-803.

In various embodiments, the expression of ARP are measured. If desired, expression of these sequences can be measured along with other sequences whose expression is known to be altered according to one of the herein described parameters or conditions.

The reference cell population includes one or more cells capable of expressing the measured ARP sequences and for which the compared parameter is known, e.g., exposed to a test agent, disease status or PPARγ expression status. By “disease status” is meant is known whether the reference cell has the disease state being screened (e.g., renal disorders such as clear cell renal carcinoma, kidney cancer, renal dyplasia, or inflammatory disorders such as allergy, asthma, emphysema. By “PPARγ expression status” is meant that is known whether the reference cell has had contact with a PPARγ ligand, e.g. N-(2-benzoylphenyl)-L-tyrosine. Whether or not comparison of the gene expression profile in the test cell population to the reference cell population reveals the presence, or degree, of the measured parameter depends on the composition of the reference cell population. For example, if the reference cell population is composed of cells that have not been treated with a known PPARγ ligand, a similar gene expression level in the test cell population and a reference cell population indicates the test agent is not a PPARγ ligand. Conversely, if the reference cell population is made up of cells that have been treated with a known PPARγ ligand, a similar gene expression profile between the test cell population and the reference cell population indicates the test agent is a PPARγ ligand.

In various embodiments, a ARP sequence in a test cell population is considered comparable in expression level to the expression level of the ARP sequence if its expression level varies within a factor of 2.0, 1.5, or 1.0 fold to the level of the ARP transcript in the reference cell population. In various embodiments, a ARP sequence in a test cell population can be considered altered in levels of expression if its expression level varies from the reference cell population by more than 1.0, 1.5, 2.0 or more fold from the expression level of the corresponding ARP sequence in the reference cell population.

If desired, comparison of differentially expressed sequences between a test cell population and a reference cell population can be done with respect to a control nucleic acid whose expression is independent of the parameter or condition being measured. Expression levels of the control nucleic acid in the test and reference nucleic acid can be used to normalize signal levels in the compared populations. Suitable control nucleic acids can readily be determined by one of ordinary skill in the art.

In some embodiments, the test cell population is compared to multiple reference cell populations. Each of the multiple reference populations may differ in the known parameter. For example, a test cell population may be compared to a first reference cell population known to have been exposed to a PPARγ ligand, as well as a second reference population known have not been exposed to a PPARγ ligand.

The test cell population that is exposed to, i.e., contacted with, the test ligand can be any number of cells, i.e., one or more cells, and can be provided in vitro, in vivo, or ex vivo.

In other embodiments, the test cell population can be divided into two or more subpopulations. The subpopulations can be created by dividing the first population of cells to create as identical a subpopulation as possible. This will be suitable, in, for example, in vitro or ex vivo screening methods. In some embodiments, various sub populations can be exposed to a control agent, and/or a test agent, multiple test agents, or, e.g., varying dosages of one or multiple test agents administered together, or in various combinations.

Preferably, cells in the reference cell population are derived from a tissue type as similar as possible to test cell, e.g., adipose tissue or liver tissue. In some embodiments, the control cell is derived from the same subject as the test cell, e.g., from a region proximal to the region of origin of the test cell. In other embodiments, the reference cell population is derived from a plurality of cells For example, the reference cell population can be a database of expression patterns from previously tested cells for which one of the herein-described parameters or conditions (e.g., PPARγ status, screening, diagnostic, or therapeutic claims) is known.

The subject is preferably a mammal. The mammal can be, e.g., a human, non-human primate, mouse, rat, dog, cat, horse, or cow.

Screening for PPARγ Ligands

In one aspect, the invention provides a method of identifying PPARγ ligands. The PPARγ ligand can be identified by providing a cell population that includes cells capable of angiopoietin related protein (ARP). The sequences need not be identical to sequences including ARP, as long as the sequence is sufficiently similar that specific hybridization can be detected. Preferably, the cell includes sequences that are identical, or nearly identical to those identifying the ARP nucleic acid or polypeptide

Expression of the nucleic acid sequences in the test cell population is then compared to the expression of the nucleic acid sequences in a reference cell population, which is a cell population that has not been exposed to the test agent, or, in some embodiments, a cell population exposed to the test agent. Comparison can be performed on test and reference samples measured concurrently or at temporally distinct times. An example of the latter is the use of compiled expression information, e.g., a sequence database, which assembles information about expression levels of known sequences following administration of various agents. For example, alteration of expression levels following administration of test agent can be compared to the expression changes observed in the nucleic acid sequences following administration of a control agent, such as N-(2-benzoylphenyl)-L-tyrosine.

Finding an alteration (e.g. increase) in the level of expression of the nucleic acid sequence in the test cell population compared to the expression of the nucleic acid sequence in the reference cell population that has not been exposed to the test agent indicates the test agent is a PPARγ ligand.

The invention also includes a PPARγ ligand identified according to this screening method, and a pharmaceutical composition comprising the PPARγ ligands so identified.

Screening Assays for Identifying a Candidate Therapeutic Agent for Treating or Preventing a Pathophysiologies Associated with the PPARγ Mediated Pathway

The differentially expressed sequences disclosed herein can also be used to identify candidate therapeutic agents pathophysiologies associated with the PPARγ mediated pathway. The method is based on screening a candidate therapeutic agent to determine if it converts an expression profile of ARP protein or nucleic acid characteristic of a PPARγ response.

In the method a cell is exposed to a test agent or a combination of test agents (sequentially or simultaneously) and the expression ARP is measured. The expression of the ARP in the test population is compared to expression level of the ARP in a reference cell population whose PPARγ status is known. If the reference cell population contains cells that have not been exposed to a PPARγ ligand, alteration of the extent of the nucleic acids in the test cell population as compared to the reference cell population indicates that the test agent is a candidate therapeutic agent.

In some embodiments, the reference cell population includes cells that have been exposed to a test agent. When this cell population is used, an alteration in expression of the nucleic acid sequences in the presence of the agent from the expression profile of the cell population in the absence of the agent indicates the agent is a candidate therapeutic agent. In other embodiments the test cell population includes cells that have not been exposed to a PPARγ ligand. For this cell population, a similarity in expression of ARP in the test and control cell populations indicates the test agent is not a candidate therapeutic agent, while a difference suggests it is a candidate.

The test agent can be a compound not previously described or can be a previously known compound but which is not known to be a PPARγ ligand

An agent effective in stimulating expression of underexpressed genes, or in suppressing expression of overexpressed genes can be further tested for its ability to prevent the PPARγ mediated pathophysiology, e.g. NIDDM, and as a potential therapeutic useful for the treatment of such pathophysiology. Further evaluation of the clinical usefulness of such a compound can be performed using standard methods of evaluating toxicity and clinical effectiveness of anti-diabetic agents.

Selecting a Therapeutic Agent for Treating a Pathophysiology Associated with the PPARγ Mediated Pathway that is Appropriate for a Particular Individual

Differences in the genetic makeup of individuals can result in differences in their relative abilities to metabolize various drugs. An agent that is metabolized in a subject to act as an PPARγ ligand can manifest itself by inducing a change in gene expression pattern in the subject's cells from that characteristic of a pathophysiologic state to a gene expression pattern characteristic of a non-pathophysiologic state. Accordingly, the differentially expressed ARP allow for a putative therapeutic or prophylactic agent to be tested in a test cell population from a selected subject in order to determine if the agent is a suitable PPARγ ligand in the subject.

To identify a PPARγ ligand, that is appropriate for a specific subject, a test cell population from the subject is exposed to a therapeutic agent, and the expression ARP is a measured.

In some embodiments, the test cell population contains a adipocyte. In other embodiments, the agent is first mixed with a cell extract, e.g., an liver cell extract or an adipose cell extract, which contains enzymes that metabolize drugs into an active form. The activated form of the therapeutic agent can then be mixed with the test cell population and gene expression measured. Preferably, the cell population is contacted ex vivo with the agent or activated form of the agent.

Expression of the nucleic acid sequences in the test cell population is then compared to the expression of the nucleic acid sequences a reference cell population. The reference cell population includes at least one cell whose PPARγ status is known. If the reference cell had been exposed to a PPARγ ligand a similar gene expression profile between the test cell population and the reference cell population indicates the agent is suitable for treating the pathophysiology in the subject. A difference in expression between sequences in the test cell population and those in the reference cell population indicates that the agent is not suitable for treating the PPARγ pathophysiology in the subject.

If the reference cell has not been exposed to a PPARγ ligand, a similarity in gene expression patterns between the test cell population and the reference cell population indicates the agent is not suitable for treating the PPARγ pathophysiology in the subject, while a dissimilar gene expression patterns indicate the agent will be suitable for treating the subject.

In some embodiments, a decrease in expression ARP or an increase in expression of one or more of ARP in a test cell population relative to a reference cell population is indicative that the agent is therapeutic.

The test agent can be any compound or composition. In some embodiments the test agents are compounds and composition know to be PPARγ ligands, e.g. N-(2-benzoylphenyl)-L-tyrosine.

Screening for Therapeutic Agents

In one aspect, the invention provides a method screening for therapeutic agents. By “therapeutic agent” is meant an agent that promotes a therapeutic effects such as a chemotherapeutic compound. Preferably, the agent promotes insulin sensitivity. More preferably the agent inhibits ARP expression or activity. The therapeutic agent can be identified by providing a cell population that includes cells capable of expressing ARP.

Expression of the nucleic acid sequences in the test cell population is then compared to the expression of the nucleic acid sequences in a reference cell population, which is a cell population that has not been exposed to the test agent, or, in some embodiments, a cell population exposed the test agent. Comparison can be performed on test and reference samples measured concurrently or at temporally distinct times. An example of the latter is the use of compiled expression information, e.g., a sequence database, which assembles information about expression levels of known sequences following administration of various agents. For example, alteration of expression levels following administration of test agent can be compared to the expression changes observed in the nucleic acid sequences following administration of a control agent, parathyroid hormone

An alteration in expression of the nucleic acid sequence in the test cell population compared to the expression of the nucleic acid sequence in the reference cell population that has not been exposed to the test agent indicates the test agent is an therapeutic agent.

The invention also includes the therapeutic agent identified according to this screening method, and a pharmaceutical composition which includes the therapeutic agent.

Methods of Diagnosing or Determining the Susceptibility to Clear Cell Renal Carcinoma in a Subject

The invention further provides a method of diagnosing a clear cell renal carcinoma, in a subject. A disorder is diagnosed by examining the expression of ARP from a test population of cells from a subject suspected of have the disorder.

Expression of ARP measured in the test cell and compared to the expression of the sequences in the reference cell population. The reference cell population contains at least one cell whose disease status (i.e., the reference cell population is from a subject suffering from a clear cell renal carcinoma) is known. If the reference cell population contains cells that have not suffering from a clear cell renal carcinoma, then a similarity in expression between ARP sequences in the test population and the reference cell population indicates the subject does not have a bone disorder. A difference (e.g., increase) in expression between ARP in the test population and the reference cell population indicates the reference cell population has clear cell renal carcinoma

Conversely, when the reference cell population contains cells that have clear cell renal carcinoma, a similarity in expression pattern between the test cell population and the reference cell population indicates the test cell population has clear cell renal carcinoma. A difference in expression between ARP sequences in the test population and the reference cell population indicates the subject does not have a clear cell renal carcinoma.

Methods of Treating Renal Disorders in a Subject

Also included in the invention is a method of treating, i.e., preventing or delaying the onset of a renal disorder in a subject by administering to the subject an agent which modulates the expression or activity of ARP “Modulates” is meant to include increase or decrease expression or activity of the ARP polypeptides or nucleic acids. Preferably, modulation results in alteration alter the expression or activity of the ARP genes or gene products in a subject to a level similar or identical to a subject not suffering from the bone disorder.

The renal disorder can be any of the pathophysiologies described herein, e.g., kidney cancer (ie., renal cell carcinoma or wilms tumor), polycystic kidney disease, renal dysplasis, kidney degenerative disease (i.e., chronic kidney failure).

In its various aspects and embodiments, the invention includes administering to a subject or contacting a cell with a compound that decrease ARP expression or activity. The compound can be, e.g., (i) an antibody or biologically active fragment thereof that specifically binds ARP; (ii) an anti-sense ARP nucleic acid; (iii) a ribozyme that specifically targets ARP (iv) a nucleic acid that decrease the expression of a nucleic acid that encodes an ARP polypeptide, and derivatives, fragments, analogs and homologs thereof and (v) small molecule ARP antagonists.

The antibody can be for example, monoclonal, polyclonal, humanized, radiolabled, or bispecific. The nucleic acid can be either endogenous or exogenous.

As used herein, the term “nucleic acid” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded. The nucleic acid can be either endogenous or exogenous. Preferably, the nucleic acid is a DNA.

The compound can be administered to the subject either directly (i.e., the subject is directly exposed to the nucleic acid or nucleic acid-containing vector) or indirectly (i.e., cells are first transformed with the nucleic acid in vitro, then transplanted into the subject). For example, in one embodiment mammalian cells are isolated from a subject and the ARP anti-sense nucleic acid is introduced into the isolated cells in vitro. The cells are reintroduced into a suitable mammalian subject. Preferably, the cell is introduced into an autologous subject. The routes of administration of the compound can include e.g., parenteral., intravenous, intradermal, subcutaneous, oral (e.g., inhalation), transdermal (topical), transmucosal, and rectal administration. In one embodiment the compound is administered intravenous.

The subject can be, e.g., a human, a rodent such as a mouse or rat, or a dog or cat.

Assessing Efficacy of Treatment of a Kidney Disorder in a Subject

The differentially expressed ARP identified herein also allow for the course of treatment of a kidney disorder to be monitored. In this method, a test cell population is provided from a subject undergoing treatment for the kidney disorder. If desired, test cell populations can be taken from the subject at various time points before, during, or after treatment. Expression of ARP in the cell population is then measured and compared to a reference cell population which includes cells whose pathophysiologic state is known. Preferably, the reference cells not been exposed to the treatment.

If the reference cell population contains no cells having the pathophysiologic state, i.e., kidney disorder, a similarity in expression between ARP in the test cell population and the reference cell population indicates that the treatment is efficacious. However, a difference in expression between ARP in the test population and this reference cell population indicates the treatment is not efficacious.

If the reference cell population contains no cells exposed to the treatment, a similarity in expression between ARP in the test cell population and the reference cell population indicates that the treatment is efficacious. However, a difference in expression between ARP in the test population and this reference cell population indicates the treatment is not efficacious.

By “efficacious” is meant that the treatment leads to a decrease in the pathophysiology in a subject. When treatment is applied prophylactically, “efficacious” means that the treatment retards or prevents a pathophysiology.

Efficaciousness can be determined in association with any known method for treating the particular pathophysiology.

Methods of Diagnosing or Determining the Susceptibility to an Inflammatory Disorder

The invention further provides a method of diagnosing an inflammatory disorder, in a subject. A disorder is diagnosed by examining the expression of ARP from a test population of cells from a subject suspected of have the disorder. An inflammatory disorder includes for example disorders of the pulmonary system, asthma, allergy, emphysema, arthritis (e.g. osteoarthritis), chronic obstructive pulmonary disease, or crohn's disease

Expression of ARP measured in the test cell and compared to the expression of the sequences in the reference cell population. The reference cell population contains at least one cell whose, or disease status (i.e., the reference cell population is from a subject suffering from an inflammatory disorder is known. If the reference cell population contains cells that have not suffering from an inflammatory disorder, then a similarity in expression between ARP sequences in the test population and the reference cell population indicates the subject does not have a bone disorder. A difference (e.g., increase) in expression between ARP in the test population and the reference cell population indicates the reference cell population has an inflammatory disorder.

Conversely, when the reference cell population contains cells that have an inflammatory disorder, a similarity in expression pattern between the test cell population and the reference cell population indicates the test cell population has an inflammatory disorder. A difference in expression between ARP sequences in the test population and the reference cell population indicates the subject does not have an inflammatory disorder.

Methods of Treating an Inflammatory in a Subject

Also included in the invention is a method of treating, i.e., preventing or delaying the onset of an inflammatory disorder in a subject by administering to the subject an agent which modulates the expression or activity of ARP “Modulates” is meant to include increase or decrease expression or activity of the ARP polypeptides or nucleic acids. Preferably, modulation results in alteration alter the expression or activity of the ARP genes or gene products in a subject to a level similar or identical to a subject not suffering from the bone disorder.

The inflammatory disorder can be any of the pathophysiologies described herein, e.g., arthritis, COPD or emphysema.

In its various aspects and embodiments, the invention includes administering to a subject or contacting a cell with a compound that decrease ARP expression or activity. The compound can be, e.g., (i) an antibody or biologically active fragment thereof that specifically binds ARP; (ii) an anti-sense ARP nucleic acid; (iii) a ribozyme that specifically targets ARP (iv) a nucleic acid that decrease the expression of a nucleic acid that encodes an ARP polypeptide, and derivatives, fragments, analogs and homologs thereof and (v) small molecule ARP antagonists.

The antibody can be for example, monoclonal, polyclonal, humanized, radiolabled, or bispecific. The nucleic acid can be either endogenous or exogenous.

As used herein, the term “nucleic acid” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded. The nucleic acid can be either endogenous or exogenous. Preferably, the nucleic acid is a DNA.

The compound can be administered to the subject either directly (ie., the subject is directly exposed to the nucleic acid or nucleic acid-containing vector) or indirectly (ie., cells are first transformed with the nucleic acid in vitro, then transplanted into the subject). For example, in one embodiment mammalian cells are isolated from a subject and the ARP anti-sense nucleic acid is introduced into the isolated cells in vitro. The cells are reintroduced into a suitable mammalian subject. Preferably, the cell is introduced into an autologous subject. The routes of administration of the compound can include e.g., parenteral., intravenous, intradernal, subcutaneous, oral (e.g., inhalation), transdermal (topical), transmucosal, and rectal administration. In one embodiment the compound is administered intravenous.

The subject can be, e.g., a human, a rodent such as a mouse or rat, or a dog or cat.

Assessing Efficacy of Treatment of an Inflammatory Disorder in a Subject

The differentially expressed ARP identified herein also allow for the course of treatment of an inflammatory disorder to be monitored. In this method, a test cell population is provided from a subject undergoing treatment for the an inflammatory disorder. If desired, test cell populations can be taken from the subject at various time points before, during, or after treatment. Expression of ARP in the cell population is then measured and compared to a reference cell population which includes cells whose pathophysiologic state is known. Preferably, the reference cells not been exposed to the treatment.

If the reference cell population contains no cells having the pathophysiologic state, i e., an inflammatory disorder, a similarity in expression between ARP in the test cell population and the reference cell population indicates that the treatment is efficacious. However, a difference in expression between ARP in the test population and this reference cell population indicates the treatment is not efficacious.

If the reference cell population contains no cells exposed to the treatment, a similarity in expression between ARP in the test cell population and the reference cell population indicates that the treatment is efficacious. However, a difference in expression between ARP in the test population and this reference cell population indicates the treatment is not efficacious.

By “efficacious” is meant that the treatment leads to a decrease in the pathophysiology in a subject. When treatment is applied prophylactically, “efficacious” means that the treatment retards or prevents a pathophysiology.

Efficaciousness can be determined in association with any known method for treating the particular pathophysiology.

Methods of Treating or Preventing Disorders

Also included in the invention are methods of treating, i.e., preventing or delaying the onset of various disorders in a subject of disorders amenable to treatment with the methods of the invention include for example, inflammatory disorders, (e.g., psoriasis, asthma, allergy, emphysema, stroke, ischemia reperfusion injury, encephalitis, meningitis, AIDS related dementia or septic shock) cancer (e.g., adenocarcinomas of the colon, squamous cell and adenocarcinomas of the lung, clear cell renal cell carcinomas, hepatocellular carcinomas, transitional cell carcinomas of the bladder, cystadenocarcinoma and adenocarcinomas of the stomach, ovarian tumors, thyroid tumors, gliomas and astrocytomas), CNS trauma (brain and spinal cord), peripheral neuropathies and demyelation diseases (e.g., multiple sclerosis, and cerebral lupus). In various aspects the method includes administering to the subject a compound which modulates the I1-8 expression or activity. “Modulates” is meant to include increase or decrease I1-8 expression or activity. Preferably, modulation results in alteration of the expression or activity of I1-8 in a subject to a level similar or identical to a subject not suffering from the disorder.

In its various aspects and embodiments, the invention includes administering to a subject or contacting a cell with a compound that decrease IL-8 expression or activity. The compound can be, e.g., (i) an antibody or biologically active fragment thereof that specifically binds IL-8; (ii) an anti-sense IL-8 nucleic acid; (iii) a ribozyme that specifically targets IL-8 (iv) a nucleic acid that decreases the expression of a nucleic acid that encodes an IL-8 polypeptide, and derivatives, fragments, analogs and homologs thereof and (v) small molecule IL-8 antagonists.

The antibody can be for example, monoclonal, polyclonal, humanized, radiolabled, or bispecific. The nucleic acid can be either endogenous or exogenous.

As used herein, the term “nucleic acid” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded. The nucleic acid can be either endogenous or exogenous. Preferably, the nucleic acid is DNA.

The compound can be administered to the subject either directly (i.e., the subject is directly exposed to the nucleic acid or nucleic acid-containing vector) or indirectly (ie., cells are first transformed with the nucleic acid in vitro, then transplanted into the subject). For example, in one embodiment mammalian cells are isolated from a subject and an IL-8 anti-sense nucleic acid is introduced into the isolated cells in vitro. The cells are reintroduced into a suitable mammalian subject. Preferably, the cell is introduced into an autologous subject. In some embodiments, the cells may also be cultured ex vivo in the presence of therapeutic agents or proteins of the present invention in order to proliferate or to produce a desired effect on or activity in such cells. Treated cells can then be introduced in vivo for therapeutic purposes.

The routes of administration of the compound can include e.g., parenteral, intravenous, intradermal, subcutaneous, oral (e.g., inhalation), transdermal (topical), transmucosal, and rectal administration. In one embodiment the compound is administered intravenously.

The subject is preferably a mammal. The mammal can be, e.g., a human, non-human primate, mouse, rat, dog, cat, horse, or cow.

The herein-described IL-8 modulating compound when used therapeutically are referred to herein as “Therapeutics”. Methods of administration of Therapeutics include, but are not limited to, intradermal, intramuscular, intraperitoneal, intravenous, subcutaneous, intranasal, epidural, and oral routes. The Therapeutics of the present invention may be administered by any convenient route, for example by infusion or bolus injection, by absorption through epithelial or mucocutaneous linings (e.g., oral mucosa, rectal and intestinal mucosa, etc.) and may be administered together with other biologically-active agents. Administration can be systemic or local. In addition, it may be advantageous to administer the Therapeutic into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by V Ad z means of a suppository, or by means of an implant. Various delivery systems are known and can be used to administer a Therapeutic of the present invention including, e.g.: (i) encapsulation in liposomes, microparticles, microcapsules; (it) recombinant cells capable of expressing the Therapeutic; (iii) receptor-mediated endocytosis (See, e.g., Wu and Wu, 1987. J Biol Chem 262:4429-4432); (iv) construction of a Therapeutic nucleic acid as part of a retroviral, adenoviral or other vector, and the like. In one embodiment of the present invention, the Therapeutic may be delivered in a vesicle, in particular a liposome. In a liposome, the protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,837,028; and U.S. Pat. No. 4,737,323, both of which are incorporated herein by reference. In yet another embodiment, the Therapeutic can be delivered in a controlled release system including, e.g. a delivery pump (See, e.g., Saudek, et al., 1989. New Engl J Med 321:574 and a semi-permeable polymeric material (See, e.g., Howard, et al., 1989. J Neurosurg 71:105). Additionally, the controlled release system can be placed in proximity of the therapeutic target (e.g., the brain), thus requiring only a fraction of the systemic dose. See, e.g., Goodson, In: Medical Applications of Controlled Release 1984. (CRC Press, Bocca Raton, Fla.).

In a specific embodiment of the present invention, where the Therapeutic is a nucleic acid encoding a protein, the Therapeutic nucleic acid may be administered in vivo to promote expression of its encoded protein, by constructing it as part of an appropriate nucleic acid expression vector and administering it so that it becomes intracellular, e.g., by use of a retroviral vector, by direct injection, by use of microparticle bombardment, by coating with lipids or cell-surface receptors or transfecting agents, or by administering it in linkage to a homeobox-like peptide which is known to enter the nucleus. See, e.g. Joliot, et al., 1991. Proc Natl Acad Sci USA 88:1864-1868. Alternatively, a nucleic acid Therapeutic can be introduced intracellularly and incorporated within host cell DNA for expression, by homologous recombination or it can remain episomal.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of the Therapeutic of the invention which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of Therapeutic of the present invention with which to treat each individual patient. Initially, the attending physician will administer low doses of Therapeutic of the present invention and observe the patient's response. Larger doses of Therapeutic of the present invention may be administered until the optimal therapeutic effect is obtained for the patient, and at that point the dosage is not increased further. However, suitable dosage ranges for intravenous administration of the Therapeutics of the present invention are generally about 20-500 micrograms (pg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient

The duration of intravenous therapy using the Therapeutic of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

The invention will be further described in the following examples, which do not limit the scope of the invention described in the claims.

### EXAMPLES

### Example 1

### Identification of NOVX Nucleic Acids

TblastN using CuraGen Corporation's sequence file for polypeptides or homologs was run against the Genomic Daily Files made available by GenBank or from files downloaded from the individual sequencing centers. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The novel NOVX target sequences identified in the present invention were subjected to the exon linking process to confirm the sequence. PCR primers were designed by starting at the most upstream sequence available, for the forward primer, and at the most downstream sequence available for the reverse primer. PCR primer sequences were used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain-whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

### Example 2

### Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8)1249-1265, 2000).

### Example 3

### Quantitative Expression analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on Nmetabolic diseases), Al_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 Ill and incubated for 60 minutes at 42° C. This reaction can be scaled up to 50 μg of total RNA in a final volume of 100 μl. sscDNA samples are then normalized to reference nucleic acids as described previously, using 1×TaqMang Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tₘ=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No.4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan* Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pi. eff=pl effusion=pleural effusion,

glio=glioma,

astro=astrocytoma, and

neuro=neuroblastoma.

General_screeningpanel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 10.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶cells/ml in DMEM 5% FCS (Hyclone), 100gM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 M non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶cellsml in DMEM 5% FCS (Hyclone), 100[M non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 g/ml anti-CD28 (Pharmingen) and 2 ng/ml OKT3 (ATCC), and then washed twice with PBS. Umbilical cord blood CD4 lymphocytes (Poietic Systems, German Town, Md.) were cultured at 10⁵-10⁶cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Trn. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr] after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵cells/ml for 8 days, changing the media every 3 days and adjusting the cell concentration to 5×10⁵cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 1 SmI Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_comprehensive panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patients 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr 2, 1999: 143-147. Clonetics provided Trizol lysates or frozen pellets suitable for mRNA isolation and ds cDNA production. A general description of each donor is as follows:

Donors 2 and 3 U: Mesenchymal Stem cells, Undifferentiated Adipose

Donors 2 and 3 AM: Adipose, AdiposeMidway Differentiated

Donosr 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supemuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA4=Brodman Area 4

Panel CNS_Neurodegeneration_V1.0

The plates for Panel CNS_Neurodegeneration_V10.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. NOV2—CG57107-01: Pepsin a Precursor

Expression of the NOV2 gene was assessed using the primer-probe set Ag809, described in Table AA. Results of the RTQ-PCR runs are shown in Tables AB, AC, AD, AE, AF, and AG.

TABLE AA

Probe Name Ag809

| | | | | |
|---|---|---|---|---|
| | | | | SEQ ID |
| Primers | Sequences | Length | Start Position | NO: |

| | | | | |
|---|---|---|---|---|
| Forward | 5′-atgtgatctttggctgtgaagt-3′ | 22 | 257 | 461 |
| Probe | TET-5′-ctaccccatggcctccatcgagt-3′-TAMRA | 23 | 228 | 462 |
| Reverse | 5′-ggatgtccaagccatcctt-3′ | 19 | 204 | 463 |

TABLE AB
General_screening_panel_v1.4

| | | | Rel. |
|---|---|---|---|
| | | | Exp. (%) |
| | Rel. Exp. (%) | | Ag809, |
| | Ag809, Run | | Run |
| Tissue Name | 220283339 | Tissue Name | 220283339 |
| Adipose | 0.7 | Renal ca. TK-10 | 6.7 |
| Melanoma* Hs688(A).T | 4.5 | Bladder | 2.7 |
| Melanoma* Hs688(B).T | 12.1 | Gastric ca. (liver met.) NCI-N87 | 7.0 |
| Melanoma* M14 | 10.7 | Gastric ca. KATO III | 10.0 |
| Melanoma* LOXIMVI | 0.8 | Colon ca. SW-948 | 2.9 |
| Melanoma* SK-MEL-5 | 0.9 | Colon ca. SW480 | 27.5 |
| Squamous cell carcinoma SCC-4 | 6.3 | Colon ca.* (SW480 met) SW620 | 18.6 |
| Testis Pool | 1.5 | Colon ca. HT29 | 3.5 |
| Prostate ca.* (bone met) PC-3 | 23.8 | Colon ca. HCT-116 | 5.0 |
| Prostate Pool | 2.7 | Colon ca. CaCo-2 | 28.5 |
| Placenta | 0.6 | Colon cancer tissue | 3.1 |
| Uterus Pool | 1.2 | Colon ca. SW1116 | 5.8 |
| Ovarian ca. OVCAR-3 | 7.7 | Colon ca. Colo-205 | 5.8 |
| Ovarian ca. SK-OV-3 | 0.4 | Colon ca. SW-48 | 2.0 |
| Ovarian ca. OVCAR-4 | 0.9 | Colon Pool | 3.0 |
| Ovarian ca. OVCAR-5 | 36.3 | Small Intestine Pool | 1.3 |
| Ovarian ca. IGROV-1 | 5.5 | Stomach Pool | 2.6 |
| Ovarian ca. OVCAR-8 | 5.5 | Bone Marrow Pool | 0.6 |
| Ovary | 0.6 | Fetal Heart | 1.6 |
| Breast ca. MCF-7 | 4.6 | Heart Pool | 1.3 |
| Breast ca. MDA-MB-231 | 7.2 | Lymph Node Pool | 4.2 |
| Breast ca. BT 549 | 17.0 | Fetal Skeletal Muscle | 4.9 |
| Breast ca. T47D | 100.0 | Skeletal Muscle Pool | 2.0 |
| Breast ca. MDA-N | 13.2 | Spleen Pool | 11.7 |
| Breast Pool | 3.2 | Thymus Pool | 4.1 |
| Trachea | 5.3 | CNS cancer (glio/astro) U87-MG | 13.0 |
| Lung | 0.5 | CNS cancer (glio/astro) U-118- | 72.2 |
| | | MG | |
| Fetal Lung | 2.9 | CNS cancer (neuro;met) SK-N- | 33.2 |
| | | AS | |
| Lung ca. NCI-N417 | 1.4 | CNS cancer (astro) SF-539 | 8.8 |
| Lung ca. LX-1 | 28.9 | CNS cancer (astro) SNB-75 | 8.8 |
| Lung ca. NCI-H146 | 1.6 | CNS cancer (glio) SNB-19 | 4.6 |
| Lung ca. SHP-77 | 7.5 | CNS cancer (glio) SF-295 | 6.2 |
| Lung ca. A549 | 4.9 | Brain (Amygdala) Pool | 0.5 |
| Lung ca. NCI-H526 | 0.8 | Brain (cerebellum) | 0.4 |
| Lung ca. NCI-H23 | 9.7 | Brain (fetal) | 0.4 |
| Lung ca. NCI-H460 | 4.5 | Brain (Hippocampus) Pool | 0.5 |
| Lung ca. HOP-62 | 1.7 | Cerebral Cortex Pool | 0.7 |
| Lung ca. NCI-H522 | 23.2 | Brain (Substantia nigra) Pool | 0.5 |
| Liver | 0.0 | Brain (Thalamus) Pool | 0.8 |
| Fetal Liver | 1.0 | Brain (whole) | 0.9 |
| Liver ca. HepG2 | 14.8 | Spinal Cord Pool | 0.4 |
| Kidney Pool | 3.6 | Adrenal Gland | 0.3 |
| Fetal Kidney | 1.6 | Pituitary gland Pool | 0.6 |
| Renal ca. 786-0 | 7.8 | Salivary Gland | 4.7 |
| Renal ca. A498 | 3.9 | Thyroid (female) | 0.6 |
| Renal ca. ACHN | 4.4 | Pancreatic ca. CAPAN2 | 5.5 |
| Renal ca. UO-31 | 0.5 | Pancreas Pool | 3.1 |

TABLE AC
Panel 1.2

| | | | | | Rel. |
|---|---|---|---|---|---|
| | Rel. | | | | Exp. (%) |
| | Exp. (%) | Rel. Exp. (%) | | Rel. Exp. (%) | Ag809, |
| | Ag809, Run | Ag809, Run | | Ag809, Run | Run |
| Tissue Name | 118348423 | 121953937 | Tissue Name | 118348423 | 121953937 |
| Endothelial cells | 0.0 | 0.0 | Renal ca. 786-0 | 5.0 | 6.7 |
| Heart (Fetal) | 15.1 | 34.6 | Renal ca. A498 | 7.7 | 12.0 |
| Pancreas | 7.4 | 0.2 | Renal ca. RXF 393 | 0.7 | 0.7 |
| Pancreatic ca. CAPAN 2 | 1.9 | 4.1 | Renal ca. ACHN | 5.1 | 7.2 |
| Adrenal Gland | 3.1 | 6.7 | Renal ca. UO-31 | 0.0 | 0.0 |
| Thyroid | 6.7 | 1.0 | Renal ca. TK-10 | 0.1 | 0.0 |
| Salivary gland | 40.3 | 63.3 | Liver | 1.1 | 1.3 |
| Pituitary gland | 16.4 | 14.1 | Liver (fetal) | 0.8 | 1.7 |
| Brain (fetal) | 0.7 | 0.0 | Liver ca. | 16.2 | 49.0 |
| | | | (hepatoblast) | | |
| | | | HepG2 | | |
| Brain (whole) | 2.1 | 3.4 | Lung | 0.5 | 1.6 |
| Brain (amygdala) | 1.0 | 1.6 | Lung (fetal) | 3.1 | 1.9 |
| Brain (cerebellum) | 0.3 | 0.7 | Lung ca. (small | 44.4 | 33.4 |
| | | | cell) LX-1 | | |
| Brain (hippocampus) | 2.7 | 6.5 | Lung ca. (small | 1.8 | 0.3 |
| | | | cell) NCI-H69 | | |
| Brain (thalamus) | 1.1 | 0.8 | Lung ca. (s. cell | 4.3 | 4.3 |
| | | | var.) SHP-77 | | |
| Cerebral Cortex | 3.4 | 8.8 | Lung ca. (large | 13.1 | 45.7 |
| | | | cell) NCI-H460 | | |
| Spinal cord | 0.7 | 0.7 | Lung ca. (non-sm. | 9.4 | 15.6 |
| | | | cell) A549 | | |
| glio/astro U87-MG | 11.4 | 8.2 | Lung ca. (non- | 11.0 | 12.9 |
| | | | s. cell) NCI-H23 | | |
| glio/astro U-118-MG | 21.2 | 24.1 | Lung ca. (non- | 6.2 | 2.7 |
| | | | s. cell) HOP-62 | | |
| astrocytoma SW1783 | 1.6 | 1.7 | Lung ca. (non-s. cl) | 81.2 | 62.4 |
| | | | NCI-H522 | | |
| neuro*; met SK-N-AS | 65.1 | 46.7 | Lung ca. (squam.) | 6.6 | 13.8 |
| | | | SW 900 | | |
| astrocytoma SF-539 | 5.6 | 9.2 | Lung ca. (squam.) | 2.0 | 0.1 |
| | | | NCI-H596 | | |
| astrocytoma SNB-75 | 0.5 | 0.0 | Mammary gland | 5.3 | 4.6 |
| glioma SNB-19 | 2.4 | 2.9 | Breast ca.* (pl. ef) | 4.2 | 6.4 |
| | | | MCF-7 | | |
| glioma U251 | 1.1 | 0.9 | Breast ca.* (pl. ef) | 2.0 | 6.0 |
| | | | MDA-MB-231 | | |
| glioma SF-295 | 2.7 | 0.5 | Breast ca.* (pl. ef) | 100.0 | 100.0 |
| | | | T47D | | |
| Heart | 39.2 | 77.4 | Breast ca. BT-549 | 3.0 | 5.7 |
| Skeletal Muscle | 52.1 | 36.3 | Breast ca. MDA-N | 17.4 | 20.4 |
| Bone marrow | 0.0 | 1.2 | Ovary | 2.1 | 3.8 |
| Thymus | 0.3 | 0.0 | Ovarian ca. | 8.4 | 4.3 |
| | | | OVCAR-3 | | |
| Spleen | 19.5 | 21.9 | Ovarian ca. | 2.3 | 3.1 |
| | | | OVCAR-4 | | |
| Lymph node | 0.3 | 6.9 | Ovarian ca. | 8.9 | 7.0 |
| | | | OVCAR-5 | | |
| Colorectal Tissue | 0.1 | 1.1 | Ovarian ca. | 4.9 | 13.1 |
| | | | OVCAR-8 | | |
| Stomach | 18.9 | 27.7 | Ovarian ca. | 7.0 | 7.7 |
| | | | IGROV-1 | | |
| Small intestine | 3.1 | 8.4 | Ovarian ca. (ascites) | 0.0 | 0.0 |
| | | | SK-OV-3 | | |
| Colon ca. SW480 | 5.2 | 8.3 | Uterus | 3.8 | 8.7 |
| Colon ca.* SW620 | 19.2 | 20.4 | Placenta | 1.9 | 3.2 |
| (SW480 met) | | | | | |
| Colon ca. HT29 | 4.1 | 2.5 | Prostate | 13.4 | 33.0 |
| Colon ca. HCT-116 | 2.0 | 1.4 | Prostate ca.* (bone | 42.9 | 76.3 |
| | | | met) PC-3 | | |
| Colon ca. CaCo-2 | 35.1 | 24.5 | Testis | 8.1 | 9.4 |
| Colon ca. Tissue | 0.6 | 3.8 | Melanoma | 5.3 | 6.0 |
| (ODO3866) | | | Hs688(A).T | | |
| Colon ca. HCC-2998 | 36.9 | 54.0 | Melanoma* (met) | 3.5 | 3.4 |
| | | | Hs688(B).T | | |
| Gastric ca.* (liver met) | 11.9 | 14.9 | Melanoma UACC- | 2.9 | 3.7 |
| NCI-N87 | | | 62 | | |
| Bladder | 7.0 | 13.8 | Melanoma M14 | 11.5 | 21.9 |
| Trachea | 6.0 | 9.3 | Melanoma LOX | 2.6 | 1.8 |
| | | | IMVI | | |
| Kidney | 2.7 | 6.0 | Melanoma* (met) | 1.7 | 3.2 |
| | | | SK-MEL-5 | | |
| Kidney (fetal) | 6.5 | 28.5 | | | |

TABLE AD
Panel 1.3D

| | Rel. Exp. (%) | | Rel. Exp. (%) |
|---|---|---|---|
| | Ag809, Run | | Ag809, Run |
| Tissue Name | 152548777 | Tissue Name | 152548777 |
| Liver adenocarcinoma | 54.3 | Kidney (fetal) | 1.3 |
| Pancreas | 1.4 | Renal ca. 786-0 | 4.8 |
| Pancreatic ca. CAPAN 2 | 3.2 | Renal ca. A498 | 4.7 |
| Adrenal gland | 1.6 | Renal ca. RXF 393 | 3.2 |
| Thyroid | 4.4 | Renal ca. ACHN | 6.7 |
| Salivary gland | 8.4 | Renal ca. UO-31 | 0.9 |
| Pituitary gland | 6.0 | Renal ca. TK-10 | 0.4 |
| Brain (fetal) | 0.0 | Liver | 0.6 |
| Brain (whole) | 2.0 | Liver (fetal) | 0.7 |
| Brain (amygdala) | 1.6 | Liver ca. (hepatoblast) HepG2 | 36.9 |
| Brain (cerebellum) | 0.0 | Lung | 2.7 |
| Brain (hippocampus) | 8.9 | Lung (fetal) | 6.1 |
| Brain (substantia nigra) | 0.6 | Lung ca. (small cell) LX-1 | 23.5 |
| Brain (thalamus) | 2.0 | Lung ca. (small cell) NCI-H69 | 2.0 |
| Cerebral Cortex | 1.6 | Lung ca. (s. cell var.) SHP-77 | 8.9 |
| Spinal cord | 1.8 | Lung ca. (large cell) NCI-H460 | 4.7 |
| glio/astro U87-MG | 8.2 | Lung ca. (non-sm. cell) A549 | 2.4 |
| glio/astro U-118-MG | 68.3 | Lung ca. (non-s. cell) NCI-H23 | 23.5 |
| astrocytoma SW1783 | 4.5 | Lung ca. (non-s. cell) HOP-62 | 7.2 |
| neuro*; met SK-N-AS | 35.6 | Lung ca. (non-s. cl) NCI-H522 | 32.5 |
| astrocytoma SF-539 | 8.6 | Lung ca. (squam.) SW 900 | 3.2 |
| astrocytoma SNB-75 | 8.1 | Lung ca. (squam.) NCI-H596 | 0.5 |
| glioma SNB-19 | 0.0 | Mammary gland | 6.8 |
| glioma U251 | 0.6 | Breast ca.* (pl .ef) MCF-7 | 3.8 |
| glioma SF-295 | 8.6 | Breast ca.* (pl. ef) MDA-MB-231 | 14.6 |
| Heart (fetal) | 29.9 | Breast ca.* (pl. ef) T47D | 45.1 |
| Heart | 9.5 | Breast ca. BT-549 | 5.2 |
| Skeletal muscle (fetal) | 100.0 | Breast ca. MDA-N | 12.8 |
| Skeletal muscle | 2.2 | Ovary | 8.2 |
| Bone marrow | 2.5 | Ovarian ca. OVCAR-3 | 2.4 |
| Thymus | 2.9 | Ovarian ca. OVCAR-4 | 2.7 |
| Spleen | 42.0 | Ovarian ca. OVCAR-5 | 6.2 |
| Lymph node | 2.3 | Ovarian ca. OVCAR-8 | 8.7 |
| Colorectal | 8.1 | Ovarian ca. IGROV-1 | 6.7 |
| Stomach | 19.3 | Ovarian ca.* (ascites) SK-OV-3 | 0.3 |
| Small intestine | 3.1 | Uterus | 4.7 |
| Colon ca. SW480 | 31.9 | Placenta | 3.2 |
| Colon ca.* SW620(SW480 met) | 12.9 | Prostate | 10.7 |
| Colon ca. HT29 | 3.6 | Prostate ca.* (bone met)PC-3 | 24.5 |
| Colon ca. HCT-116 | 3.4 | Testis | 9.9 |
| Colon ca. CaCo-2 | 33.7 | Melanoma Hs688(A).T | 9.2 |
| Colon ca. tissue(ODO3866) | 2.0 | Melanoma* (met) Hs688(B).T | 48.6 |
| Colon ca. HCC-2998 | 18.9 | Melanoma UACC-62 | 1.2 |
| Gastric ca.* (liver met) NCI-N87 | 7.2 | Melanoma M14 | 6.5 |
| Bladder | 2.5 | Melanoma LOX IMVI | 1.6 |
| Trachea | 16.0 | Melanoma* (met) SK-MEL-5 | 2.3 |
| Kidney | 1.2 | Adipose | 4.0 |

TABLE AE
Panel 2D

| | | | Rel. |
|---|---|---|---|
| | | | Exp. (%) |
| | Rel. Exp. (%) | | Ag809, |
| | Ag809, Run | | Run |
| Tissue Name | 152550394 | Tissue Name | 152550394 |
| Normal Colon | 6.8 | Kidney Margin 8120608 | 1.5 |
| CC Well to Mod Diff (ODO3866) | 6.1 | Kidney Cancer 8120613 | 2.0 |
| CC Margin (ODO3866) | 2.5 | Kidney Margin 8120614 | 4.1 |
| CC Gr. 2 rectosigmoid (ODO3868) | 0.9 | Kidney Cancer 9010320 | 2.2 |
| CC Margin (ODO3868) | 1.2 | Kidney Margin 9010321 | 3.5 |
| CC Mod Diff (ODO3920) | 3.8 | Normal Uterus | 3.1 |
| CC Margin (ODO3920) | 1.3 | Uterus Cancer 064011 | 17.6 |
| CC Gr. 2 ascend colon (ODO3921) | 6.9 | Normal Thyroid | 3.7 |
| CC Margin (ODO3921) | 4.0 | Thyroid Cancer 064010 | 1.2 |
| CC from Partial Hepatectomy | 1.2 | Thyroid Cancer A302152 | 0.6 |
| (ODO4309) Mets | | | |
| Liver Margin (ODO4309) | 0.6 | Thyroid Margin A302153 | 2.6 |
| Colon mets to lung (OD04451-01) | 4.4 | Normal Breast | 3.3 |
| Lung Margin (OD04451-02) | 1.2 | Breast Cancer (OD04566) | 0.9 |
| Normal Prostate 6546-1 | 10.2 | Breast Cancer (OD04590-01) | 67.8 |
| Prostate Cancer (OD04410) | 41.8 | Breast Cancer Mets | 51.1 |
| | | (OD04590-03) | |
| Prostate Margin (OD04410) | 25.7 | Breast Cancer Metastasis | 12.7 |
| | | (OD04655-05) | |
| Prostate Cancer (OD04720-01) | 11.0 | Breast Cancer 064006 | 8.9 |
| Prostate Margin (OD04720-02) | 10.0 | Breast Cancer 1024 | 7.8 |
| Normal Lung 061010 | 7.9 | Breast Cancer 9100266 | 6.2 |
| Lung Met to Muscle (ODO4286) | 6.5 | Breast Margin 9100265 | 3.3 |
| Muscle Margin (ODO4286) | 2.6 | Breast Cancer A209073 | 3.4 |
| Lung Malignant Cancer (OD03126) | 14.8 | Breast Margin A209073 | 8.7 |
| Lung Margin (OD03126) | 3.1 | Normal Liver | 1.1 |
| Lung Cancer (OD04404) | 2.0 | Liver Cancer 064003 | 0.6 |
| Lung Margin (OD04404) | 1.9 | Liver Cancer 1025 | 0.6 |
| Lung Cancer (OD04565) | 0.3 | Liver Cancer 1026 | 1.4 |
| Lung Margin (OD04565) | 1.9 | Liver Cancer 6004-T | 1.3 |
| Lung Cancer (OD04237-01) | 1.3 | Liver Tissue 6004-N | 1.3 |
| Lung Margin (OD04237-02) | 2.6 | Liver Cancer 6005-T | 1.1 |
| Ocular Mel Met to Liver (ODO4310) | 0.1 | Liver Tissue 6005-N | 0.3 |
| Liver Margin (ODO4310) | 0.6 | Normal Bladder | 5.9 |
| Melanoma Mets to Lung (OD04321) | 2.5 | Bladder Cancer 1023 | 1.7 |
| Lung Margin (OD04321) | 2.6 | Bladder Cancer A302173 | 1.9 |
| Normal Kidney | 5.6 | Bladder Cancer (OD04718-01) | 2.0 |
| Kidney Ca, Nuclear grade 2 (OD04338) | 0.6 | Bladder Normal Adjacent | 3.3 |
| | | (OD04718-03) | |
| Kidney Margin (OD04338) | 3.7 | Normal Ovary | 2.2 |
| Kidney Ca Nuclear grade ½ | 0.8 | Ovarian Cancer 064008 | 29.1 |
| (OD04339) | | | |
| Kidney Margin (OD04339) | 3.1 | Ovarian Cancer (OD04768-07) | 100.0 |
| Kidney Ca, Clear cell type (OD04340) | 1.5 | Ovary Margin (OD04768-08) | 2.2 |
| Kidney Margin (OD04340) | 5.1 | Normal Stomach | 13.1 |
| Kidney Ca, Nuclear grade 3 (OD04348) | 14.5 | Gastric Cancer 9060358 | 1.3 |
| Kidney Margin (OD04348) | 2.5 | Stomach Margin 9060359 | 8.8 |
| Kidney Cancer (OD04622-01) | 1.7 | Gastric Cancer 9060395 | 2.5 |
| Kidney Margin (OD04622-03) | 2.0 | Stomach Margin 9060394 | 9.7 |
| Kidney Cancer (OD04450-01) | 0.3 | Gastric Cancer 9060397 | 15.9 |
| Kidney Margin (OD04450-03) | 2.0 | Stomach Margin 9060396 | 12.9 |
| Kidney Cancer 8120607 | 7.0 | Gastric Cancer 064005 | 12.1 |

TABLE AF
Panel 4.1D

| | Rel. Exp. (%) Ag809, | | Rel. Exp. (%) Ag809, |
|---|---|---|---|
| Tissue Name | Run 170402442 | Tissue Name | Run 170402442 |
| Secondary Th1 act | 2.5 | HUVEC IL-1beta | 0.9 |
| Secondary Th2 act | 2.7 | HUVEC IFN gamma | 3.0 |
| Secondary Tr1 act | 3.8 | HUVEC TNF alpha + IFN | 0.9 |
| | | gamma | |
| Secondary Th1 rest | 1.8 | HUVEC TNF alpha + IL4 | 0.7 |
| Secondary Th2 rest | 0.0 | HUVEC IL-11 | 2.5 |
| Secondary Tr1 rest | 0.0 | Lung Microvascular EC none | 2.5 |
| Primary Th1 act | 0.0 | Lung Microvascular EC | 1.4 |
| | | TNFalpha + IL-1beta | |
| Primary Th2 act | 0.4 | Microvascular Dermal EC | 0.0 |
| | | none | |
| Primary Tr1 act | 2.3 | Microsvasular Dermal EC | 2.3 |
| | | TNFalpha + IL-1beta | |
| Primary Th1 rest | 4.4 | Bronchial epithelium | 2.5 |
| | | TNFalpha + IL1beta | |
| Primary Th2 rest | 1.4 | Small airway epithelium | 3.2 |
| | | none | |
| Primary Tr1 rest | 1.7 | Small airway epithelium | 0.0 |
| | | TNFalpha + IL-1beta | |
| CD45RA CD4 | 14.9 | Coronery artery SMC rest | 1.5 |
| lymphocyte act | | | |
| CD45RO CD4 | 0.7 | Coronery artery SMC | 2.7 |
| lymphocyte act | | TNFalpha + IL-1beta | |
| CD8 lymphocyte act | 0.7 | Astrocytes rest | 1.7 |
| Secondary CD8 | 0.8 | Astrocytes TNFalpha + IL- | 1.4 |
| lymphocyte rest | | 1beta | |
| Secondary CD8 | 2.3 | KU-812 (Basophil) rest | 5.7 |
| lymphocyte act | | | |
| CD4 lymphocyte none | 0.0 | KU-812 (Basophil) | 7.1 |
| | | PMA/ionomycin | |
| 2ry Th1/Th2/Tr1_anti- | 0.0 | CCD1106 (Keratinocytes) | 3.3 |
| CD95 CH11 | | none | |
| LAK cells rest | 3.2 | CCD1106 (Keratinocytes) | 0.5 |
| | | TNFalpha + IL-1beta | |
| LAK cells IL-2 | 2.1 | Liver cirrhosis | 3.3 |
| LAK cells IL-2 + IL-12 | 1.3 | NCI-H292 none | 36.6 |
| LAK cells IL-2 + IFN | 1.2 | NCI-H292 IL-4 | 38.7 |
| gamma | | | |
| LAK cells IL-2 + IL-18 | 0.7 | NCI-H292 IL-9 | 46.7 |
| LAK cells | 0.9 | NCI-H292 1L-13 | 37.4 |
| PMA/ionomycin | | | |
| NK Cells IL-2 rest | 0.9 | NCI-H292 IFN gamma | 53.2 |
| Two Way MLR 3 day | 1.8 | HPAEC none | 4.0 |
| Two Way MLR 5 day | 1.6 | HPAEC TNF alpha + IL-1 | 2.2 |
| | | beta | |
| Two Way MLR 7 day | 2.9 | Lung fibroblast none | 62.4 |
| PBMC rest | 0.6 | Lung fibroblast TNF alpha + | 33.2 |
| | | IL-1 beta | |
| PBMC PWM | 1.1 | Lung fibroblast IL-4 | 100.0 |
| PBMC PHA-L | 3.1 | Lung fibroblast IL-9 | 86.5 |
| Ramos (B cell) none | 0.6 | Lung fibroblast IL-13 | 66.0 |
| Ramos (B cell) | 4.9 | Lung fibroblast IFN gamma | 84.1 |
| ionomycin | | | |
| B lymphocytes PWM | 2.0 | Dermal fibroblast CCD1070 | 90.8 |
| | | rest | |
| B lymphocytes CD40L | 1.1 | Dermal fibroblast CCD1070 | 25.7 |
| and IL-4 | | TNF alpha | |
| EOL-1 dbcAMP | 12.7 | Dermal fibroblast CCD1070 | 40.3 |
| | | IL-1 beta | |
| EOL-1 dbcAMP | 4.0 | Dermal fibroblast IFN | 37.4 |
| PMA/ionomycin | | gamma | |
| Dendritic cells none | 5.8 | Dermal fibroblast IL-4 | 71.2 |
| Dendritic cells LPS | 2.8 | Dermal Fibroblasts rest | 20.3 |
| Dendritic cells anti- | 1.4 | Neutrophils TNFa + LPS | 4.3 |
| CD40 | | | |
| Monocytes rest | 2.0 | Neutrophils rest | 0.0 |
| Monocytes LPS | 0.9 | Colon | 9.2 |
| Macrophages rest | 3.8 | Lung | 0.0 |
| Macrophages LPS | 3.3 | Thymus | 36.1 |
| HUVEC none | 0.4 | Kidney | 99.3 |
| HUVEC starved | 4.7 | | |

TABLE AG
Panel 4D

| | Rel. Exp. (%) Ag809, | | Rel. Exp. (%) Ag809, |
|---|---|---|---|
| Tissue Name | Run 152552244 | Tissue Name | Run 152552244 |
| Secondary Th1 act | 2.0 | HUVEC IL-1beta | 1.2 |
| Secondary Th2 act | 1.5 | HUVEC IFN gamma | 1.4 |
| Secondary Tr1 act | 2.5 | HUVEC TNF alpha + IFN | 0.8 |
| | | gamma | |
| Secondary Th1 rest | 1.0 | HUVEC TNF alpha + IL4 | 1.1 |
| Secondary Th2 rest | 3.0 | HUVEC IL-11 | 3.0 |
| Secondary Tr1 rest | 1.7 | Lung Microvascular EC none | 0.8 |
| Primary Th1 act | 0.4 | Lung Microvascular EC | 0.5 |
| | | TNFalpha + IL-1beta | |
| Primary Th2 act | 1.5 | Microvascular Dermal EC | 1.1 |
| | | none | |
| Primary Tr1 act | 2.0 | Microsvasular Dermal EC | 1.0 |
| | | TNFalpha + IL-1beta | |
| Primary Th1 rest | 5.4 | Bronchial epithelium | 0.0 |
| | | TNFalpha + IL1beta | |
| Primary Th2 rest | 3.1 | Small airway epithelium | 0.4 |
| | | none | |
| Primary Tr1 rest | 0.0 | Small airway epithelium | 0.5 |
| | | TNFalpha + IL-1beta | |
| CD45RA CD4 | 11.2 | Coronery artery SMC rest | 5.8 |
| lymphocyte act | | | |
| CD45RO CD4 | 1.2 | Coronery artery SMC | 2.3 |
| lymphocyte act | | TNFalpha + IL-1beta | |
| CD8 lymphocyte act | 0.9 | Astrocytes rest | 2.7 |
| Secondary CD8 | 0.0 | Astrocytes TNFalpha + IL- | 0.0 |
| lymphocyte rest | | 1beta | |
| Secondary CD8 | 0.6 | KU-812 (Basophil) rest | 6.8 |
| lymphocyte act | | | |
| CD4 lymphocyte none | 1.1 | KU-812 (Basophil) | 8.4 |
| | | PMA/ionomycin | |
| 2ry Th1/Th2/Tr1_anti- | 0.0 | CCD1106 (Keratinocytes) | 1.6 |
| CD95 CH11 | | none | |
| LAK cells rest | 0.5 | CCD1106 (Keratinocytes) | 1.4 |
| | | TNFalpha + IL-1beta | |
| LAK cells IL-2 | 0.0 | Liver cirrhosis | 4.2 |
| LAK cells IL-2 + IL-12 | 0.7 | Lupus kidney | 1.8 |
| LAK cells IL-2 + IFN | 1.1 | NCI-H292 none | 39.5 |
| gamma | | | |
| LAK cells IL-2 + IL-18 | 0.3 | NCI-H292 IL-4 | 39.0 |
| LAK cells | 0.0 | NCI-H292 IL-9 | 65.5 |
| PMA/ionomycin | | | |
| NK Cells IL-2 rest | 1.3 | NCI-H292 IL-13 | 37.1 |
| Two Way MLR 3 day | 0.5 | NCI-H292 IFN gamma | 31.9 |
| Two Way MLR 5 day | 0.5 | HPAEC none | 0.5 |
| Two Way MLR 7 day | 2.6 | HPAEC TNF alpha + IL-1 | 1.2 |
| | | beta | |
| PBMC rest | 0.0 | Lung fibroblast none | 42.3 |
| PBMC PWM | 1.3 | Lung fibroblast TNF alpha + | 17.8 |
| | | IL-1 beta | |
| PBMC PHA-L | 1.0 | Lung fibroblast IL-4 | 100.0 |
| Ramos (B cell) none | 1.2 | Lung fibroblast IL-9 | 72.7 |
| Ramos (B cell) | 2.3 | Lung fibroblast IL-13 | 60.7 |
| ionomycin | | | |
| B lymphocytes PWM | 4.3 | Lung fibroblast IFN gamma | 81.8 |
| B lymphocytes CD40L | 1.4 | Dermal fibroblast CCD1070 | 76.8 |
| and IL-4 | | rest | |
| EOL-1 dbcAMP | 7.2 | Dermal fibroblast CCD1070 | 30.1 |
| | | TNF alpha | |
| EOL-1 dbcAMP | 3.0 | Dermal fibroblast CCD1070 | 38.2 |
| PMA/ionomycin | | IL-1 beta | |
| Dendritic cells none | 1.5 | Dermal fibroblast IFN | 34.2 |
| | | gamma | |
| Dendritic cells LPS | 0.7 | Dermal fibroblast IL-4 | 80.7 |
| Dendritic cells anti- | 0.5 | IBD Colitis 2 | 0.3 |
| CD40 | | | |
| Monocytes rest | 0.5 | IBD Crohn's | 1.4 |
| Monocytes LPS | 0.0 | Colon | 35.6 |
| Macrophages rest | 1.3 | Lung | 11.0 |
| Macrophages LPS | 1.7 | Thymus | 5.8 |
| HUVEC none | 2.3 | Kidney | 9.7 |
| HUVEC starved | 9.0 | | |

General_screening_panelv1.4 Summary: Ag809 Highest expression of the NOV2 gene is seen in a breast cancer cell line (CT=27.2). Significant expression is also seen in a cluster of cell lines derived from breast cancer, colon cancer and brain cancer. Thus, expression of this gene could be used to differentiate between these samples and other samples on this panel and as a marker to detect the presence of breast, colon, and brain cancer. Furthermore, therapeutic modulation of the expression or function of this gene may be effective in the treatment of breast, colon, and brain cancers.

Panel 1.2 Summary: Ag809 Two experiment with the same probe and primer set produce results that are in excellent agreement, with highest expression of the NOV2 gene in a breast cancer cell line (CTs=26-27). In addition, significant expression is also seen in most cancer cell lines in this panel, including prostate, brain, colon, ovarian, liver and lung cancers. Thus, expression of this gene could be used to differentiate between these sample and other samples on this panel and as a marker to detect the presence of cancer. Furthermore, therapeutic modulation of the expression or function of this gene may be effective in the treatment of prostate, brain, colon, ovarian, liver and lung cancers.

Among tissues with metabolic function, this gene is expressed at moderate to low levels in pituitary, adrenal gland, pancreas, thyroid, skeletal muscle and adult and fetal heart and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This gene also exhibits moderate expression in the brain, especially in the hippocampus. The hippocampus is a region of specific neurodegeneration in Alzheimer's disease, that is thought to be mediated by the amyloid precursor protein processing enzyme, beta secretase. Beta secretase is a drug target of utility in the treatment of Alzheimer's disease. Since both this gene product and beta secretase are aspartyl proteases, the protein encoded by this gene may have potential utility as a drug target to treat Alzheimeres disease.

### REFERENCES

Mallender W D, Yager D, Onstead L, Nichols M R, Eckman C, Sambamurti K, Kopcho L M, Marcinkeviciene J, Copeland R A, Rosenberry T L. Characterization of recombinant, soluble beta-secretase from an insect cell expression system. Mol Pharmacol March 2001;59(3):619-26

The beta-site amyloid precursor protein-cleaving enzyme (BACE) cleaves the amyloid precursor protein to produce the N terminus of the amyloid beta peptide, a major component of the plaques found in the brains of Alzheimer's disease patients. Sequence analysis of BACE indicates that the protein contains the consensus sequences found in most known aspartyl proteases, but otherwise has only modest homology with aspartyl proteases of known three-dimensional structure (i.e., pepsin, renin, or cathepsin D). Because BACE has been shown to be one of the two proteolytic activities responsible for the production of the Abeta peptide, this enzyme is a prime target for the design of therapeutic agents aimed at reducing Abeta for the treatment of Alzheimeres disease. Toward this ultimate goal, we have expressed a recombinant, truncated human BACE in a Drosophila melanogaster S2 cell expression system to generate high levels of secreted BACE protein. The protein was convenient to purify and was enzymatically active and specific for cleaving the beta-secretase site of human APP, as demonstrated with soluble APP as the substrate in novel sandwich enzyme-linked immunosorbent assay and Western blot assays. Further kinetic analysis revealed no catalytic differences between this recombinant, secreted BACE, and brain BACE. Both showed a strong preference for substrates that contained the Swedish mutation, where NL is substituted for KM immediately upstream of the cleavage site, relative to the wild-type sequence, and both showed the same extent of inhibition by a peptide-based inhibitor. The capability to produce large quantities of BACE enzyme will facilitate protein structure determination and inhibitor development efforts that may lead to the evolution of useful Alzheimer's disease treatments.

Panel 1.3D Summary: Ag809 Highest expression of the CG57107-01 gene is seen in fetal skeletal muscle (CT=29.6). This gene also has low levels of expression in thyroid, pituitary, adult and fetal heart, and adipose. This widespread expression in tissues of metabolic origin suggests that this gene product may be a small molecule target for the treatment of endocrine or metabolic disease, including thyroidopathies and obesity.

Significant expression of this gene is also seen in brain, colon, lung and breast cancer cell lines as well as a melanoma cell line. This prominent expression in cancer cell lines is consistent with expression in Panels 1.2 and 2D. Therefore, expression of this gene could be used as a diagnostic marker for cancers of these tissues. Furthermore, therapeutic modulation of the gene product using antibodies and small molecule drugs may be used for the treatment of these cancers.

This gene also shows low levels of expression in the CNS. Please see Panel 1.2 for discussion of utility of this gene in the central nervous system.

Panel 2D Summary: Ag809 The CG57107-01 gene is expressed at a higher level in prostate, ovarian and breast cancer compared to the adjacent normal tissues. Therefore, expression of this gene could be used as a diagnostic marker for the presence of these cancers. Furthermore, therapeutic inhibition of the gene product using antibodies and small molecule drugs may be useful for the treatment of these cancers.

Panel 3D Summary: Ag809 Results from one experiment with the CG57107-01 gene are not included. The amp plot indicates that there were experimental difficulties with this run.

Panels 4D/4.1D Summary: Ag809 Significant expression of the CG57107-01 gene is limited to fibroblast and NCI-H292 cells (CTs=31-33). Expression is also seen in normal thymus and kidney. Therefore, expression of this transcript or the protein it encodes could be used as a marker for these tissues. In addition, therapeutics designed with the protein encoded by this transcript could be used to regulate the expression of this putative enzyme.

B. NOV3—CG56936-01: Ribonuclease Pancreatic-Like Protein

Expression of the NOV3 gene was assessed using the primer-probe set Ag2477, described in Table BA. Results of the RTQ-PCR runs are shown in Tables BB and BC.

TABLE BA

Probe Name Ag2477

| | | | | |
|---|---|---|---|---|
| | | | | SEQ ID |
| Primers | Sequences | Length | Start Position | NO: |

| | | | | |
|---|---|---|---|---|
| Forward | 5′-ctgcaaccacatgatcatacaa-3′ | 22 | 273 | 464 |
| Probe | TET-5′-atcagggaacctgaccacacttgtaa-3′-TAMRA | 26 | 241 | 465 |
| Reverse | 5′-atggatgaagacatgctccttt-3′ | 22 | 219 | 466 |

TABLE BB
Panel 1.3D

| | | | Rel. Exp. (%) |
|---|---|---|---|
| | Rel. Exp. (%) Ag2477, Run | | Ag2477, Run |
| Tissue Name | 165639391 | Tissue Name | 165639391 |
| Liver adenocarcinoma | 0.0 | Kidney (fetal) | 0.0 |
| Pancreas | 0.0 | Renal ca. 786-0 | 0.0 |
| Pancreatic ca. CAPAN2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) LX-1 | 0.0 |
| Brain (thalamus) | 0.0 | Lung ca. (small cell) | 0.0 |
| | | NCI-H69 | |
| Cerebral Cortex | 0.0 | Lung ca. (s. cell var.) | 18.8 |
| | | SHP-77 | |
| Spinal cord | 0.0 | Lung ca. (large cell)NCI- | 0.0 |
| | | H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) NCI- | 0.0 |
| | | H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U251 | 7.5 | Breast ca.* (pl. ef) MCF-7 | 0.0 |
| glioma SF-295 | 13.7 | Breast ca.* (pl. ef) MDA- | 0.0 |
| | | MB-231 | |
| Heart (fetal) | 0.0 | Breast ca.* (pl. ef) T47D | 0.0 |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca.* (ascites) | 0.0 |
| | | SK-OV-3 | |
| Small intestine | 0.0 | Uterus | 0.0 |
| Colon ca. SW480 | 0.0 | Placenta | 0.0 |
| Colon ca.* SW620(SW480 | 0.0 | Prostate | 0.0 |
| met) | | | |
| Colon ca. HT29 | 0.0 | Prostate ca.* (bone | 0.0 |
| | | met)PC-3 | |
| Colon ca. HCT-116 | 0.0 | Testis | 100.0 |
| Colon ca. CaCo-2 | 0.0 | Melanoma Hs688(A).T | 0.0 |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX IMVI | 0.0 |
| Trachea | 9.1 | Melanoma* (met) SK- | 0.0 |
| | | MEL-5 | |
| Kidney | 0.0 | Adipose | 0.0 |

TABLE BC
Panel 4D

| | Rel. Exp. (%) Ag2477, | | Rel. Exp. (%) Ag2477, |
|---|---|---|---|
| Tissue Name | Run 164391869 | Tissue Name | Run 164391869 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 35.8 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 17.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN | 10.3 |
| | | gamma | |
| Dendritic cells LPS | 0.0 | Dermal fibroblast IL-4 | 0.0 |
| Dendritic cells anti- | 0.0 | IBD Colitis 2 | 22.8 |
| CD40 | | | |
| Monocytes rest | 0.0 | IBD Crohn's | 0.0 |
| Monocytes LPS | 0.0 | Colon | 0.0 |
| Macrophages rest | 0.0 | Lung | 0.0 |
| Macrophages LPS | 0.0 | Thymus | 8.0 |
| HUVEC none | 0.0 | Kidney | 0.0 |
| HUVEC starved | 0.0 | | |

Panel 1.3D Summary: Ag2477 Significant expression of the NOV3 gene is restricted to the testis (CT=33.1). Thus, expression of this gene could be used to differentiate testis tissue from other tissues. Furthermore, the highly specific expression of the NOV3 gene suggests that its protein product may be involved in the normal function of the testis. Thus, therapeutic modulation of the expression or function of this gene may be useful in the treatment of infertility and other disorders that involve the testis.

Panel 4D Summary: Ag 2477 The NOV3 transcript is expressed almost exclusively in liver cirrhosis (CT=33.5) but not in normal liver. The protein encoded for by this transcript may be involved or associated with the pathology of this tissue and may serve as a diagnostic marker for liver cirrhosis or other inflammatory liver diseases.

C. NOV4—CG51707-02: SER/THR Protein Kinase

Expression of the NOV4 gene was assessed using the primer-probe sets Ag2827 and Ag3274, described in Tables CA and CB. Results of the RTQ-PCR runs are shown in Tables CC, CD, and CE.

TABLE CA

Probe Name Ag2827

| | | | | |
|---|---|---|---|---|
| | | | | SEQ ID |
| Primers | Sequences | Length | Start Position | NO: |

| | | | | |
|---|---|---|---|---|
| Forward | 5′-actggtgctgaagatcatgagt-3′ | 22 | 670 | 467 |
| Probe | TET-5′-cacctttgcacctatctctgaccggt-3′-TAMRA | 26 | 694 | 468 |
| Reverse | 5′-aggctccaggctgagtagact-3′ | 21 | 749 | 469 |

TABLE CB

Probe Name Ag3274

| | | | | |
|---|---|---|---|---|
| | | | | SEQ ID |
| Primers | Sequences | Length | Start Position | NO: |

| | | | | |
|---|---|---|---|---|
| Forward | 5′-tacgagaacttcctggaagaca-3′ | 22 | 239 | 470 |
| Probe | TET-5′-aagcccttatgaccgccatggaatat-3′-TAMRA | 26 | 261 | 471 |
| Reverse | 5′-attacagcgcttttggatgaa-3′ | 21 | 311 | 472 |

TABLE CC
Panel 1.3D

| | Rel. Exp. (%) | | Rel. Exp. (%) |
|---|---|---|---|
| | Ag2827, Run | | Ag2827, Run |
| Tissue Name | 165528176 | Tissue Name | 165528176 |
| Liver adenocarcinoma | 31.9 | Kidney (fetal) | 51.4 |
| Pancreas | 17.9 | Renal ca. 786-0 | 18.4 |
| Pancreatic ca. CAPAN2 | 10.8 | Renal ca. A498 | 39.2 |
| Adrenal gland | 19.1 | Renal ca. RXF 393 | 5.9 |
| Thyroid | 40.1 | Renal ca. ACHN | 24.7 |
| Salivary gland | 37.9 | Renal ca. UO-31 | 14.7 |
| Pituitary gland | 52.1 | Renal ca. TK-10 | 3.9 |
| Brain (fetal) | 3.7 | Liver | 12.1 |
| Brain (whole) | 7.2 | Liver (fetal) | 58.2 |
| Brain (amygdala) | 23.7 | Liver ca. (hepatoblast) | 95.9 |
| | | HepG2 | |
| Brain (cerebellum) | 6.9 | Lung | 14.0 |
| Brain (hippocampus) | 16.7 | Lung (fetal) | 71.2 |
| Brain (substantia nigra) | 6.7 | Lung ca. (small cell) | 25.7 |
| | | LX-1 | |
| Brain (thalamus) | 32.8 | Lung ca. (small cell) | 14.3 |
| | | NCI-H69 | |
| Cerebral Cortex | 14.6 | Lung ca. (s. cell var.) | 8.0 |
| | | SHP-77 | |
| Spinal cord | 22.1 | Lung ca. (large | 45.1 |
| | | cell)NCI-H460 | |
| glio/astro U87-MG | 12.0 | Lung ca. (non-sm. cell) | 14.5 |
| | | A549 | |
| glio/astro U-118-MG | 28.3 | Lung ca. (non-s. cell) | 21.2 |
| | | NCI-H23 | |
| astrocytoma SW1783 | 11.3 | Lung ca. (non-s. cell) | 27.4 |
| | | HOP-62 | |
| neuro*; met SK-N-AS | 6.7 | Lung ca. (non-s. cl) NCI- | 6.0 |
| | | H522 | |
| astrocytoma SF-539 | 28.1 | Lung ca. (squam.) SW | 17.6 |
| | | 900 | |
| astrocytoma SNB-75 | 17.8 | Lung ca. (squam.) NCI- | 22.4 |
| | | H596 | |
| glioma SNB-19 | 12.9 | Mammary gland | 24.0 |
| glioma U251 | 17.1 | Breast ca.* (pl. ef) MCF-7 | 36.6 |
| glioma SF-295 | 24.7 | Breast ca.* (pl. ef) | 36.9 |
| | | MDA-MB-231 | |
| Heart (fetal) | 0.0 | Breast ca.* (pl. ef) T47D | 52.1 |
| Heart | 2.1 | Breast ca. BT-549 | 21.2 |
| Skeletal muscle (fetal) | 9.2 | Breast ca. MDA-N | 0.0 |
| Skeletal muscle | 6.6 | Ovary | 2.3 |
| Bone marrow | 24.1 | Ovarian ca. OVCAR-3 | 7.6 |
| Thymus | 22.4 | Ovarian ca. OVCAR-4 | 0.0 |
| Spleen | 13.6 | Ovarian ca. OVCAR-5 | 27.0 |
| Lymph node | 100.0 | Ovarian ca. OVCAR-8 | 4.5 |
| Colorectal | 7.1 | Ovarian ca. IGROV-1 | 6.0 |
| Stomach | 19.8 | Ovarian ca.* (ascites) | 27.0 |
| | | SK-OV-3 | |
| Small intestine | 39.2 | Uterus | 24.7 |
| Colon ca. SW480 | 9.3 | Placenta | 42.9 |
| Colon ca.* | 14.5 | Prostate | 29.7 |
| SW620(SW480 met) | | | |
| Colon ca. HT29 | 10.7 | Prostate ca.* (bone | 13.2 |
| | | met)PC-3 | |
| Colon ca. HCT-116 | 6.6 | Testis | 20.7 |
| Colon ca. CaCo-2 | 20.4 | Melanoma Hs688(A).T | 7.2 |
| Colon ca. | 6.9 | Melanoma* (met) | 0.0 |
| tissue(ODO3866) | | Hs688(B).T | |
| Colon ca. HCC-2998 | 33.7 | Melanoma UACC-62 | 16.2 |
| Gastric ca.* (liver met) | 42.9 | Melanoma M14 | 9.0 |
| NCI-N87 | | | |
| Bladder | 35.4 | Melanoma LOX IMVI | 12.1 |
| Trachea | 48.3 | Melanoma* (met) SK- | 10.0 |
| | | MEL-5 | |
| Kidney | 39.5 | Adipose | 4.2 |

TABLE CD
Panel 2D

| | Rel. Exp. (%) Ag2827, | | Rel. Exp. (%) Ag2827, |
|---|---|---|---|
| Tissue Name | Run 162599361 | Tissue Name | Run 162599361 |
| Normal Colon | 38.4 | Kidney Margin | 18.6 |
| | | 8120608 | |
| CC Well to Mod Diff | 7.8 | Kidney Cancer 8120613 | 6.0 |
| (ODO3866) | | | |
| CC Margin (ODO3866) | 9.6 | Kidney Margin | 25.0 |
| | | 8120614 | |
| CC Gr. 2 rectosigmoid | 4.9 | Kidney Cancer 9010320 | 11.2 |
| (ODO3868) | | | |
| CC Margin (ODO3868) | 0.4 | Kidney Margin | 24.7 |
| | | 9010321 | |
| CC Mod Diff (ODO3920) | 49.0 | Normal Uterus | 3.2 |
| CC Margin (ODO3920) | 14.2 | Uterus Cancer 064011 | 21.6 |
| CC Gr. 2 ascend colon | 33.0 | Normal Thyroid | 22.5 |
| (ODO3921) | | | |
| CC Margin (ODO3921) | 6.0 | Thyroid Cancer 064010 | 22.5 |
| CC from Partial | 48.6 | Thyroid Cancer | 30.1 |
| Hepatectomy (ODO4309) | | A302152 | |
| Mets | | | |
| Liver Margin (ODO4309) | 19.2 | Thyroid Margin | 39.0 |
| | | A302153 | |
| Colon mets to lung | 17.1 | Normal Breast | 21.9 |
| (OD04451-01) | | | |
| Lung Margin (OD04451-02) | 11.2 | Breast Cancer | 29.5 |
| | | (OD04566) | |
| Normal Prostate 6546-1 | 100.0 | Breast Cancer | 82.9 |
| | | (OD04590-01) | |
| Prostate Cancer (OD04410) | 36.1 | Breast Cancer Mets | 76.3 |
| | | (OD04590-03) | |
| Prostate Margin (OD04410) | 47.0 | Breast Cancer | 49.7 |
| | | Metastasis (OD04655- | |
| | | 05) | |
| Prostate Cancer (OD04720- | 39.5 | Breast Cancer 064006 | 21.8 |
| 01) | | | |
| Prostate Margin (OD04720- | 49.7 | Breast Cancer 1024 | 32.1 |
| 02) | | | |
| Normal Lung 061010 | 37.1 | Breast Cancer 9100266 | 52.9 |
| Lung Met to Muscle | 11.6 | Breast Margin 9100265 | 21.5 |
| (ODO4286) | | | |
| Muscle Margin (ODO4286) | 3.6 | Breast Cancer A209073 | 15.4 |
| Lung Malignant Cancer | 30.6 | Breast Margin A209073 | 20.3 |
| (OD03126) | | | |
| Lung Margin (OD03126) | 27.0 | Normal Liver | 12.8 |
| Lung Cancer (OD04404) | 9.5 | Liver Cancer 064003 | 5.5 |
| Lung Margin (OD04404) | 20.0 | Liver Cancer 1025 | 8.7 |
| Lung Cancer (OD04565) | 9.2 | Liver Cancer 1026 | 11.4 |
| Lung Margin (OD04565) | 25.7 | Liver Cancer 6004-T | 7.9 |
| Lung Cancer (OD04237-01) | 40.6 | Liver Tissue 6004-N | 15.6 |
| Lung Margin (OD04237-02) | 17.1 | Liver Cancer 6005-T | 17.0 |
| Ocular Mel Met to Liver | 22.8 | Liver Tissue 6005-N | 7.0 |
| (ODO4310) | | | |
| Liver Margin (ODO4310) | 12.6 | Normal Bladder | 33.0 |
| Melanoma Mets to Lung | 10.3 | Bladder Cancer 1023 | 6.6 |
| (OD04321) | | | |
| Lung Margin (OD04321) | 27.4 | Bladder Cancer | 5.8 |
| | | A302173 | |
| Normal Kidney | 66.9 | Bladder Cancer | 39.2 |
| | | (OD04718-01) | |
| Kidney Ca, Nuclear grade 2 | 82.9 | Bladder Normal | 13.6 |
| (OD04338) | | Adjacent (OD04718-03) | |
| Kidney Margin (OD04338) | 54.0 | Normal Ovary | 5.7 |
| Kidney Ca Nuclear grade | 93.3 | Ovarian Cancer 064008 | 18.4 |
| 1/2 (OD04339) | | | |
| Kidney Margin (OD04339) | 61.6 | Ovarian Cancer | 39.5 |
| | | (OD04768-07) | |
| Kidney Ca, Clear cell type | 20.7 | Ovary Margin | 6.3 |
| (OD04340) | | (OD04768-08) | |
| Kidney Margin (OD04340) | 48.6 | Normal Stomach | 12.3 |
| Kidney Ca, Nuclear grade 3 | 28.9 | Gastric Cancer 9060358 | 3.6 |
| (OD04348) | | | |
| Kidney Margin (OD04348) | 80.1 | Stomach Margin | 12.6 |
| | | 9060359 | |
| Kidney Cancer (OD04622- | 9.5 | Gastric Cancer 9060395 | 11.4 |
| 01) | | | |
| Kidney Margin (OD04622- | 13.0 | Stomach Margin | 14.1 |
| 03 | | 9060394 | |
| Kidney Cancer (OD04450- | 57.8 | Gastric Cancer 9060397 | 22.5 |
| 01) | | | |
| Kidney Margin (OD04450- | 38.2 | Stomach Margin | 10.5 |
| 03) | | 9060396 | |
| Kidney Cancer 8120607 | 33.4 | Gastric Cancer 064005 | 20.0 |

TABLE CE
Panel 4D

| | | | Rel. Exp. (%) |
|---|---|---|---|
| | Rel. Exp. (%) Ag2827, | | Ag2827, Run |
| Tissue Name | Run 162294650 | Tissue Name | 162294650 |
| Secondary Th1 act | 3.1 | HUVEC IL-1beta | 0.7 |
| Secondary Th2 act | 7.8 | HUVEC IFN gamma | 3.1 |
| Secondary Tr1 act | 7.0 | HUVEC TNF alpha + IFN | 8.8 |
| | | gamma | |
| Secondary Th1 rest | 2.7 | HUVEC TNF alpha + IL4 | 5.0 |
| Secondary Th2 rest | 3.6 | HUVEC IL-11 | 2.7 |
| Secondary Tr1 rest | 2.5 | Lung Microvascular EC none | 8.1 |
| Primary Th1 act | 9.1 | Lung Microvascular EC | 3.8 |
| | | TNFalpha + IL-1beta | |
| Primary Th2 act | 12.2 | Microvascular Dermal EC none | 7.5 |
| Primary Tr1 act | 6.4 | Microsvasular Dermal EC | 8.5 |
| | | TNFalpha + IL-1beta | |
| Primary Th1 rest | 16.5 | Bronchial epithelium TNFalpha + | 10.7 |
| | | IL1beta | |
| Primary Th2 rest | 6.7 | Small airway epithelium none | 4.7 |
| Primary Tr1 rest | 7.1 | Small airway epithelium | 11.8 |
| | | TNFalpha + IL-1beta | |
| CD45RA CD4 | 8.0 | Coronery artery SMC rest | 3.8 |
| lymphocyte act | | | |
| CD45RO CD4 | 6.9 | Coronery artery SMC | 3.6 |
| lymphocyte act | | TNFalpha + IL-1beta | |
| CD8 lymphocyte act | 7.6 | Astrocytes rest | 3.3 |
| Secondary CD8 | 11.7 | Astrocytes TNFalpha + 1L- | 5.1 |
| lymphocyte rest | | 1beta | |
| Secondary CD8 | 9.8 | KU-812 (Basophil) rest | 9.0 |
| lymphocyte act | | | |
| CD4 lymphocyte none | 4.6 | KU-812 (Basophil) | 16.7 |
| | | PMA/ionomycin | |
| 2ry Th1/Th2/Tr1_anti- | 8.0 | CCD1106 (Keratinocytes) none | 9.7 |
| CD95 CH11 | | | |
| LAK cells rest | 7.3 | CCD1106 (Keratinocytes) | 7.6 |
| | | TNFalpha + IL-1beta | |
| LAK cells IL-2 | 9.3 | Liver cirrhosis | 5.0 |
| LAK cells IL-2 + IL-12 | 10.7 | Lupus kidney | 2.6 |
| LAK cells IL-2 + IFN | 17.8 | NCI-H292 none | 20.3 |
| gamma | | | |
| LAK cells IL-2 + IL-18 | 13.4 | NCI-H292 IL-4 | 20.3 |
| LAK cells | 9.2 | NCI-H292 IL-9 | 31.2 |
| PMA/ionomycin | | | |
| NK Cells IL-2 rest | 9.6 | NCI-H292 IL-13 | 22.8 |
| Two Way MLR 3 day | 15.0 | NCI-H292 IFN gamma | 27.7 |
| Two Way MLR 5 day | 7.0 | HPAEC none | 2.5 |
| Two Way MLR 7 day | 3.7 | HPAEC TNF alpha + IL-1 beta | 4.6 |
| PBMC rest | 7.4 | Lung fibroblast none | 6.6 |
| PBMC PWM | 27.5 | Lung fibroblast TNF alpha + | 2.6 |
| | | IL-1 beta | |
| PBMC PHA-L | 11.6 | Lung fibroblast IL-4 | 13.2 |
| Ramos (B cell) none | 39.2 | Lung fibroblast IL-9 | 6.6 |
| Ramos (B cell) ionomycin | 100.0 | Lung fibroblast IL-13 | 3.1 |
| B lymphocytes PWM | 33.9 | Lung fibroblast IFN gamma | 7.1 |
| B lymphocytes CD40L | 49.0 | Dermal fibroblast CCD1070 | 4.2 |
| and IL-4 | | rest | |
| EOL-1 dbcAMP | 31.4 | Dermal fibroblast CCD1070 | 8.0 |
| | | TNF alpha | |
| EOL-1 dbcAMP | 6.2 | Dermal fibroblast CCD1070 IL- | 2.3 |
| PMA/ionomycin | | 1 beta | |
| Dendritic cells none | 3.4 | Dermal fibroblast IFN gamma | 4.2 |
| Dendritic cells LPS | 3.1 | Dermal fibroblast IL-4 | 9.5 |
| Dendritic cells anti-CD40 | 2.8 | IBD Colitis 2 | 0.8 |
| Monocytes rest | 11.2 | IBD Crohn's | 3.2 |
| Monocytes LPS | 11.7 | Colon | 15.2 |
| Macrophages rest | 8.3 | Lung | 7.9 |
| Macrophages LPS | 8.4 | Thymus | 25.3 |
| HUVEC none | 2.3 | Kidney | 7.3 |
| HUVEC starved | 7.5 | | |

CNS_neurodegeneration_v1.0 Summary: Ag2827/Ag3274 Expression of the NOV4 gene is low/undetectable in all samples on this panel (CTs>35). (Data not shown.) The amp plot indicates that the experiment with the probe and primer set Ag3274 shows high probability of a probe failure.

Panel 1.3D Summary: Ag2827 Expression of the NOV4 gene is restricted to lymph node and a liver cancer cell line (CTs=34). Thus, expression of the NOV4 gene could be used to differentiate between these samples and other samples on this panel and as a marker for lymph tissue and liver cancer. A second experiment with the probe/primer set Ag3110 shows low/undetectable levels of expression in all samples on this panel (CTs>35). (Data not shown.)

Panel 2D Summary: Ag2827 Highest expression of the NOV4 gene is seen in normal prostate (CT=31). Significant expression is also seen in normal colon and a cluster of breast cancer cell lines. Thus, expression of the NOV4 gene could be used to differentiate between these samples and other samples on this panel.

Panel 4D Summary: Ag2827 Widespread expression of the NOV4 gene is seen in this panel, with highest expression in the B cell line Ramos treated with ionomycin (CT=30.8). This transcript encodes a kinase-like molecule with potential signaling activity and thus may be important in maintaining normal cellular functions in a number of tissues. Therefore, therapies designed with the protein encoded by this transcript may be important in regulating cellular viability or function.

D. NOV6—CG56684-02: Glycodelin

Expression of the NOV6 gene was assessed using the primer-probe sets Ag2994 and Ag2974, described in Tables DA and DB. Results of the RTQ-PCR runs are shown in Table DC.

TABLE DA

Probe Name Ag2994

| | | | | |
|---|---|---|---|---|
| Primers | Sequences | Length | Start Position | SEQ ID NO: |

| | | | | |
|---|---|---|---|---|
| Forward | 5′-acaaggtcatggaggaattcat-3′ | 22 | 454 | 473 |
| Probe | TET-5′-agctttctcaggaccctgcccgt-3′-TAMRA | 23 | 477 | 474 |
| Reverse | 5′-tgggtaacgtccaggaagat-3′ | 20 | 510 | 475 |

TABLE DB

Probe Name Ag2974

| | | | | |
|---|---|---|---|---|
| Primers | Sequences | Length | Start Position | SEQ ID NO: |

| | | | | |
|---|---|---|---|---|
| Forward | 5′-acaaggtcatggaggaattcat-3′ | 22 | 454 | 476 |
| Probe | TET-5′-agctttctcaggaccctgcccgt-3′-TAMRA | 23 | 477 | 477 |
| Reverse | 5′-tgggtaacgtccaggaagat-3′ | 20 | 510 | 478 |

TABLE DC
Panel 4D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag2974, | Ag2974, | Ag2994, | | Ag2974, | Ag2974, | Ag2994, |
| | Run | Run | Run | | Run | Run | Run |
| Tissue Name | 164521104 | 164535592 | 164404075 | Tissue Name | 164521104 | 164535592 | 164404075 |
| Secondary Th1 act | 0.0 | 0.0 | 0.0 | HUVEC IL-1beta | 0.0 | 0.0 | 0.0 |
| Secondary Th2 act | 0.0 | 0.0 | 0.0 | HUVEC IFN | 0.0 | 0.0 | 0.0 |
| | | | | gamma | | | |
| Secondary Tr1 act | 0.0 | 0.0 | 0.0 | HUVEC TNF alpha + | 0.0 | 0.0 | 0.0 |
| | | | | IFN gamma | | | |
| Secondary Th1 | 0.0 | 0.0 | 0.0 | HUVEC TNF alpha + | 0.0 | 0.0 | 0.0 |
| rest | | | | IL4 | | | |
| Secondary Th2 | 0.9 | 0.0 | 0.0 | HUVEC IL-11 | 0.0 | 18.8 | 0.0 |
| rest | | | | | | | |
| Secondary Tr1 | 0.0 | 0.0 | 0.0 | Lung Microvascular | 27.5 | 48.0 | 11.6 |
| rest | | | | EC none | | | |
| Primary Th1 act | 0.0 | 0.0 | 0.0 | Lung Microvascular | 0.7 | 0.0 | 0.0 |
| | | | | EC TNFalpha + IL- | | | |
| | | | | 1beta | | | |
| Primary Th2 act | 0.0 | 0.0 | 0.0 | Microvascular | 0.0 | 13.6 | 23.3 |
| | | | | Dermal EC none | | | |
| Primary Tr1 act | 0.0 | 0.0 | 0.0 | Microsvasular | 12.9 | 32.3 | 10.7 |
| | | | | Dermal EC | | | |
| | | | | TNFalpha + IL- | | | |
| | | | | 1beta | | | |
| Primary Th1 rest | 0.0 | 0.0 | 0.0 | Bronchial | 0.0 | 0.0 | 0.0 |
| | | | | epithelium | | | |
| | | | | TNFalpha + IL1beta | | | |
| Primary Th2 rest | 0.0 | 0.0 | 0.0 | Small airway | 0.0 | 0.0 | 0.0 |
| | | | | epithelium none | | | |
| Primary Tr1 rest | 0.0 | 0.0 | 0.0 | Small airway | 0.0 | 0.0 | 0.0 |
| | | | | epithelium | | | |
| | | | | TNFalpha + IL- | | | |
| | | | | 1beta | | | |
| CD45RA CD4 | 0.0 | 0.0 | 0.0 | Coronery artery | 0.0 | 0.0 | 0.0 |
| lymphocyte act | | | | SMC rest | | | |
| CD45RO CD4 | 0.0 | 0.0 | 0.0 | Coronery artery | 0.0. | 0.0 | 0.0 |
| lymphocyte act | | | | SMC TNFalpha + | | | |
| | | | | IL-1beta | | | |
| CD8 lymphocyte | 0.0 | 0.0 | 0.0 | Astrocytes rest | 0.0 | 40.1 | 0.0 |
| act | | | | | | | |
| Secondary CD8 | 0.0 | 0.0 | 0.0 | Astrocytes | 0.0 | 0.0 | 0.0 |
| lymphocyte rest | | | | TNFalpha + IL- | | | |
| | | | | 1beta | | | |
| Secondary CD8 | 0.0 | 0.0 | 0.0 | KU-812 (Basophil) | 0.0 | 0.0 | 0.0 |
| lymphocyte act | | | | rest | | | |
| CD4 lymphocyte | 0.0 | 0.0 | 0.0 | KU-812 (Basophil) | 0.0 | 0.0 | 0.0 |
| none | | | | PMA/ionomycin | | | |
| 2ry | 0.0 | 0.0 | 0.0 | CCD1106 | 0.0 | 0.0 | 0.0 |
| Th1/Th2/Tr1_anti- | | | | (Keratinocytes) | | | |
| CD95 CH11 | | | | none | | | |
| LAK cells rest | 0.0 | 0.0 | 0.0 | CCD1106 | 0.0 | 0.0 | 0.0 |
| | | | | (Keratinocytes) | | | |
| | | | | TNFalpha + IL- | | | |
| | | | | 1beta | | | |
| LAK cells IL-2 | 0.0 | 0.0 | 0.0 | Liver cirrhosis | 100.0 | 100.0 | 99.3 |
| LAK cells IL- | 0.0 | 0.0 | 0.0 | Lupus kidney | 0.0 | 0.0 | 0.0 |
| 2 + IL-12 | | | | | | | |
| LAK cells IL- | 0.0 | 0.0 | 0.0 | NCI-H292 none | 0.0 | 0.0 | 0.0 |
| 2 + IFN gamma | | | | | | | |
| LAK cells IL-2 + | 0.0 | 0.0 | 0.0 | NCI-H292 IL-4 | 0.0 | 0.0 | 0.0 |
| IL-18 | | | | | | | |
| LAK cells | 0.0 | 0.0 | 0.0 | NCI-H292 IL-9 | 0.0 | 0.0 | 0.0 |
| PMA/ionomycin | | | | | | | |
| NK Cells IL-2 rest | 0.0 | 0.0 | 0.0 | NCI-H292 IL-13 | 0.0 | 0.0 | 0.0 |
| Two Way MLR 3 | 0.0 | 0.0 | 0.0 | NCI-H292 IFN | 0.0 | 0.0 | 0.0 |
| day | | | | gamma | | | |
| Two Way MLR 5 | 0.0 | 0.0 | 0.0 | HPAEC none | 0.0 | 0.0 | 0.0 |
| day | | | | | | | |
| Two Way MLR 7 | 0.0 | 0.0 | 0.0 | HPAEC TNF alpha + | 0.0 | 26.2 | 49.3 |
| day | | | | IL-1 beta | | | |
| PBMC rest | 0.0 | 0.0 | 0.0 | Lung fibroblast | 0.0 | 0.0 | 0.0 |
| | | | | none | | | |
| PBMC PWM | 0.0 | 0.0 | 0.0 | Lung fibroblast | 0.0 | 0.0 | 0.0 |
| | | | | TNF alpha + IL-1 | | | |
| | | | | beta | | | |
| PBMC PHA-L | 0.0 | 0.0 | 0.0 | Lung fibroblast IL-4 | 0.0 | 0.0 | 0.0 |
| Ramos (B cell) | 0.0 | 19.1 | 0.0 | Lung fibroblast IL-9 | 0.0 | 0.0 | 0.0 |
| none | | | | | | | |
| Ramos (B cell) | 6.4 | 11.7 | 27.2 | Lung fibroblast IL- | 5.3 | 0.0 | 0.0 |
| ionomycin | | | | 13 | | | |
| B lymphocytes | 0.0 | 0.0 | 0.0 | Lung fibroblast IFN | 0.0 | 0.0 | 0.0 |
| PWM | | | | gamma | | | |
| B lymphocytes | 0.0 | 0.0 | 0.0 | Dermal fibroblast | 0.0 | 0.0 | 0.0 |
| CD40L and IL-4 | | | | CCD1070 rest | | | |
| EOL-1 dbcAMP | 0.0 | 0.0 | 0.0 | Dermal fibroblast | 0.0 | 0.0 | 0.0 |
| | | | | CCD1070 TNF | | | |
| | | | | alpha | | | |
| EOL-1 dbcAMP | 0.0 | 0.0 | 0.0 | Dermal fibroblast | 0.0 | 0.0 | 0.0 |
| PMA/ionomycin | | | | CCD1070 IL-1 beta | | | |
| Dendritic cells | 0.0 | 0.0 | 0.0 | Dermal fibroblast | 0.0 | 0.0 | 0.0 |
| none | | | | IFN gamma | | | |
| Dendritic cells | 0.0 | 0.0 | 0.0 | Dermal fibroblast | 0.0 | 0.0 | 0.0 |
| LPS | | | | IL-4 | | | |
| Dendritic cells | 0.0 | 0.0 | 0.0 | IBD Colitis 2 | 0.0 | 17.3 | 0.0 |
| anti-CD40 | | | | | | | |
| Monocytes rest | 0.0 | 0.0 | 0.0 | IBD Crohn's | 0.0 | 0.0 | 0.0 |
| Monocytes LPS | 0.0 | 0.0 | 0.0 | Colon | 60.7 | 96.6 | 89.5 |
| Macrophages rest | 0.0 | 0.0 | 0.0 | Lung | 14.9 | 41.8 | 100.0 |
| Macrophages LPS | 0.0 | 0.0 | 0.0 | Thymus | 0.0 | 15.3 | 12.1 |
| HUVEC none | 0.0 | 0.0 | 0.0 | Kidney | 0.0 | 0.0 | 0.0 |
| HUVEC starved | 0.0 | 0.0 | 0.0 | | | | |

CNS neurodegeneration_v1.0 Summary: Ag2994 Expression of the NOV6 gene is low/undetectable in all samples on this panel (CTs>35). (Data not shown.)

Panel 1.3D Summary: Ag2994 Expression of the NOV6 gene is low/undetectable in all samples on this panel (CTs>35). (Data not shown.) Results from a second experiment with the probe/primer set Ag2974 are not included. The amp plot indicates that there were experimental difficulties with this run.

Panel 4D Summary: Ag2974/Ag2994 Three experiments with the same probe and primer set all show significant expression of the NOV6 gene restricted to colon, lung, and liver cirrhosis. Thus, expression of the NOV6 gene could be used as a marker for colon and lung tissue and liver cirrhosis. Furthermore, expression of this gene is decreased in colon samples from patients with IBD colitis and Crohn's disease relative to normal colon. Therefore, therapeutic modulation of the activity of the protein encoded by this gene may be useful in the treatment of inflammatory bowel disease. In addition, antibodies or small molecule therapeutics may reduce or inhibit fibrosis that occurs in liver cirrhosis.

E. NOV7—CG56977-01: Neuropathy Target Esterase/Swiss Cheese

Expression of NOV7 gene was assessed using the primer-probe sets Ag3055 and Ag3061, described in Tables EA and EB. Results of the RTQ-PCR runs are shown in Tables EC, ED, EE and EF.

TABLE EA

Probe Name Ag3055

| | | | | |
|---|---|---|---|---|
| Primers | Sequences | Length | Start Position | SEQ ID NO: |

| | | | | |
|---|---|---|---|---|
| Forward | 5′-cctcatccttttcatgttcaga-3′ | 22 | 81 | 479 |
| Probe | TET-5′-actcctcagtaccggttccggaagag-3′-TAMRA | 26 | 133 | 480 |
| Reverse | 5′-gccgtaaaacatcactttgtct-3′ | 22 | 159 | 481 |

TABLE EB

Probe Name Ag3O61

| | | | | |
|---|---|---|---|---|
| Primers | Sequences | Length | Start Position | SEQ ID NO: |

| | | | | |
|---|---|---|---|---|
| Forward | 5′-cctcatccttttcatgttcaga3′ | 22 | 81 | 482 |
| Probe | TET-5′-actcctcagtaccggttccggaagag-3′-TAMRA | 26 | 133 | 483 |
| Reverse | 5′-gccgtaaaacatcactttgtct-3′ | 22 | 159 | 484 |

TABLE EC
Panel 1.3D

| | Rel. | | | Rel. | Rel. |
|---|---|---|---|---|---|
| | Exp. (%) | | | Exp. (%) | Exp. (%) |
| | Ag3055, | Rel. Exp. (%) | | Ag3055, | Ag3061, |
| | Run | Ag3061, Run | | Run | Run |
| Tissue Name | 167985388 | 167960032 | Tissue Name | 167985388 | 167960032 |
| Liver | 3.8 | 11.8 | Kidney (fetal) | 25.3 | 39.0 |
| adenocarcinoma | | | | | |
| Pancreas | 22.1 | 20.6 | Renal ca. 786-0 | 2.2 | 3.6 |
| Pancreatic ca. | 2.1 | 2.8 | Renal ca. A498 | 3.1 | 5.5 |
| CAPAN 2 | | | | | |
| Adrenal gland | 12.9 | 9.7 | Renal ca. RXF 393 | 2.6 | 4.6 |
| Thyroid | 11.7 | 11.7 | Renal ca. ACHN | 1.8 | 1.4 |
| Salivary gland | 6.8 | 11.2 | Renal ca. UO-31 | 0.6 | 0.6 |
| Pituitary gland | 18.0 | 20.0 | Renal ca. TK-10 | 1.9 | 5.0 |
| Brain (fetal) | 30.1 | 23.7 | Liver | 1.9 | 4.1 |
| Brain (whole) | 25.0 | 41.5 | Liver (fetal) | 2.7 | 7.6 |
| Brain (amygdala) | 19.3 | 37.9 | Liver ca. (hepatoblast) | 1.9 | 4.6 |
| | | | HepG2 | | |
| Brain (cerebellum) | 15.7 | 24.3 | Lung | 3.6 | 6.3 |
| Brain (hippocampus) | 26.6 | 34.4 | Lung (fetal) | 18.3 | 22.8 |
| Brain (substantia | 13.5 | 18.6 | Lung ca. (small cell) LX-1 | 2.6 | 8.9 |
| nigra) | | | | | |
| Brain (thalamus) | 17.0 | 9.4 | Lung ca. (small cell) NCI- | 3.5 | 4.1 |
| | | | H69 | | |
| Cerebral Cortex | 36.6 | 55.1 | Lung ca. (s. cell var.) SHP- | 2.3 | 6.3 |
| | | | 77 | | |
| Spinal cord | 9.7 | 19.6 | Lung ca. (large cell) NCI- | 0.4 | 0.0 |
| | | | H460 | | |
| glio/astro U87-MG | 8.9 | 2.2 | Lung ca. (non-sm. cell) | 5.1 | 9.0 |
| | | | A549 | | |
| glio/astro U-118-MG | 1.2 | 0.4 | Lung ca. (non-s. cell) NCI- | 2.6 | 4.9 |
| | | | H23 | | |
| astrocytoma | 3.7 | 5.6 | Lung ca. (non-s. cell) HOP- | 1.2 | 1.4 |
| SW1783 | | | 62 | | |
| neuro*; met SK-N- | 4.0 | 7.3 | Lung ca. (non-s. cl) NCI- | 8.6 | 9.1 |
| AS | | | H522 | | |
| astrocytoma SF-539 | 1.1 | 0.8 | Lung ca. (squam.) SW 900 | 1.6 | 2.2 |
| astrocytoma SNB-75 | 2.8 | 5.0 | Lung ca. (squam.) NCI- | 2.1 | 5.2 |
| | | | H596 | | |
| glioma SNB-19 | 1.4 | 2.8 | Mammary gland | 11.4 | 10.7 |
| glioma U251 | 4.8 | 6.3 | Breast ca.* (pl. ef) MCF-7 | 19.6 | 23.0 |
| glioma SF-295 | 5.1 | 9.4 | Breast ca.* (pl. ef) MDA- | 0.4 | 2.0 |
| | | | MB-231 | | |
| Heart (fetal) | 37.6 | 43.8 | Breast ca.* (pl. ef) T47D | 100.0 | 100.0 |
| Heart | 7.6 | 15.5 | Breast ca. BT-549 | 0.3 | 0.7 |
| Skeletal muscle | 37.4 | 44.4 | Breast ca. MDA-N | 5.0 | 8.6 |
| (fetal) | | | | | |
| Skeletal muscle | 32.8 | 48.3 | Ovary | 12.3 | 11.5 |
| Bone marrow | 1.8 | 1.8 | Ovarian ca. OVCAR-3 | 1.6 | 2.8 |
| Thymus | 17.6 | 12.4 | Ovarian ca. OVCAR-4 | 0.0 | 0.0 |
| Spleen | 5.8 | 11.0 | Ovarian ca. OVCAR-5 | 12.2 | 8.1 |
| Lymph node | 20.2 | 42.3 | Ovarian ca. OVCAR-8 | 1.9 | 2.7 |
| Colorectal | 11.1 | 16.7 | Ovarian ca. IGROV-1 | 0.5 | 0.0 |
| Stomach | 20.6 | 22.8 | Ovarian ca.* (ascites) SK- | 6.6 | 4.9 |
| | | | OV-3 | | |
| Small intestine | 6.5 | 6.2 | Uterus | 24.7 | 36.1 |
| Colon ca. SW480 | 2.1 | 3.8 | Placenta | 2.5 | 1.1 |
| Colon ca.* | 6.1 | 11.7 | Prostate | 18.8 | 28.3 |
| SW620 (SW480 met) | | | | | |
| Colon ca. HT29 | 1.7 | 3.2 | Prostate ca.* (bone | 4.2 | 2.8 |
| | | | met) PC-3 | | |
| Colon ca. HCT-116 | 1.8 | 1.2 | Testis | 5.4 | 8.6 |
| Colon ca. CaCo-2 | 2.8 | 4.7 | Melanoma Hs688(A).T | 0.6 | 0.4 |
| Colon ca. | 1.1 | 2.2 | Melanoma* (met) | 1.8 | 2.3 |
| tissue (ODO3866) | | | Hs688(B).T | | |
| Colon ca. HCC-2998 | 3.9 | 5.7 | Melanoma UACC-62 | 4.2 | 3.0 |
| Gastric ca.* (liver | 4.8 | 11.1 | Melanoma M14 | 0.5 | 3.5 |
| met) NCI-N87 | | | | | |
| Bladder | 24.0 | 28.9 | Melanoma LOX IMVI | 0.9 | 0.9 |
| Trachea | 8.4 | 8.4 | Melanoma* (met) SK- | 4.8 | 1.9 |
| | | | MEL-5 | | |
| Kidney | 12.0 | 11.7 | Adipose | 22.1 | 27.2 |

TABLE ED
Panel 2.2

| | Rel. Exp. (%) Ag3055, | | Rel. Exp. (%) Ag3055, |
|---|---|---|---|
| Tissue Name | Run 173763014 | Tissue Name | Run 173763014 |
| Normal Colon | 18.9 | Kidney Margin | 56.6 |
| | | (OD04348) | |
| Colon cancer (OD06064) | 0.0 | Kidney malignant cancer | 0.0 |
| | | (OD06204B) | |
| Colon Margin (OD06064) | 1.7 | Kidney normal adjacent | 8.7 |
| | | tissue (OD06204E) | |
| Colon cancer (OD06159) | 0.0 | Kidney Cancer | 3.7 |
| | | (OD04450-01) | |
| Colon Margin (OD06159) | 17.0 | Kidney Margin | 15.1 |
| | | (OD04450-03) | |
| Colon cancer (OD06297- | 1.1 | Kidney Cancer 8120613 | 2.5 |
| 04) | | | |
| Colon Margin (OD06297- | 13.8 | Kidney Margin 8120614 | 13.7 |
| 05) | | | |
| CC Gr. 2 ascend colon | 5.0 | Kidney Cancer 9010320 | 1.2 |
| (ODO3921) | | | |
| CC Margin (ODO3921) | 1.9 | Kidney Margin 9010321 | 5.1 |
| Colon cancer metastasis | 0.0 | Kidney Cancer 8120607 | 5.5 |
| (OD06104) | | | |
| Lung Margin (OD06104) | 2.0 | Kidney Margin 8120608 | 2.9 |
| Colon mets to lung | 2.1 | Normal Uterus | 24.1 |
| (OD04451-01) | | | |
| Lung Margin (OD04451- | 11.7 | Uterine Cancer 064011 | 19.3 |
| 02) | | | |
| Normal Prostate | 32.3 | Normal Thyroid | 5.3 |
| Prostate Cancer | 12.2 | Thyroid Cancer 064010 | 12.2 |
| (OD04410) | | | |
| Prostate Margin | 29.5 | Thyroid Cancer A302152 | 37.1 |
| (OD04410) | | | |
| Normal Ovary | 10.1 | Thyroid Margin A302153 | 5.0 |
| Ovarian cancer | 0.0 | Normal Breast | 35.6 |
| (OD06283-03) | | | |
| Ovarian Margin | 2.4 | Breast Cancer (OD04566) | 8.4 |
| (OD06283-07) | | | |
| Ovarian Cancer 064008 | 8.8 | Breast Cancer 1024 | 33.7 |
| Ovarian cancer | 14.5 | Breast Cancer (OD04590- | 18.4 |
| (OD06145) | | 01) | |
| Ovarian Margin | 16.0 | Breast Cancer Mets | 19.3 |
| (OD06145) | | (OD04590-03) | |
| Ovarian cancer | 4.4 | Breast Cancer Metastasis | 33.0 |
| (OD06455-03) | | (OD04655-05) | |
| Ovarian Margin | 5.4 | Breast Cancer 064006 | 22.2 |
| (OD06455-07) | | | |
| Normal Lung | 15.7 | Breast Cancer 9100266 | 5.6 |
| Invasive poor diff. lung | 10.5 | Breast Margin 9100265 | 7.6 |
| adeno (ODO4945-01 | | | |
| Lung Margin (ODO4945- | 24.5 | Breast Cancer A209073 | 2.2 |
| 03) | | | |
| Lung Malignant Cancer | 1.5 | Breast Margin A2090734 | 41.2 |
| (OD03126) | | | |
| Lung Margin (OD03126) | 10.6 | Breast cancer (OD06083) | 17.8 |
| Lung Cancer (OD05014A) | 3.1 | Breast cancer node | 17.4 |
| | | metastasis (OD06083) | |
| Lung Margin (OD05014B) | 17.9 | Normal Liver | 36.1 |
| Lung cancer (OD06081) | 1.8 | Liver Cancer 1026 | 2.1 |
| Lung Margin (OD06081) | 5.7 | Liver Cancer 1025 | 37.4 |
| Lung Cancer (OD04237- | 0.0 | Liver Cancer 6004-T | 22.4 |
| 01) | | | |
| Lung Margin (OD04237- | 10.0 | Liver Tissue 6004-N | 12.0 |
| 02) | | | |
| Ocular Melanoma | 12.8 | Liver Cancer 6005-T | 6.5 |
| Metastasis | | | |
| Ocular Melanoma Margin | 12.2 | Liver Tissue 6005-N | 42.9 |
| (Liver) | | | |
| Melanoma Metastasis | 4.5 | Liver Cancer 064003 | 9.3 |
| Melanoma Margin (Lung) | 4.8 | Normal Bladder | 22.5 |
| Normal Kidney | 18.3 | Bladder Cancer 1023 | 5.3 |
| Kidney Ca, Nuclear grade | 44.1 | Bladder Cancer A302173 | 1.0 |
| 2 (OD04338) | | | |
| Kidney Margin | 15.2 | Normal Stomach | 60.3 |
| (OD04338) | | | |
| Kidney Ca Nuclear grade | 100.0 | Gastric Cancer 9060397 | 0.0 |
| 1/2 (OD04339) | | | |
| Kidney Margin | 21.6 | Stomach Margin 9060396 | 11.0 |
| (OD04339) | | | |
| Kidney Ca, Clear cell type | 16.3 | Gastric Cancer 9060395 | 8.3 |
| (OD04340) | | | |
| Kidney Margin | 19.6 | Stomach Margin 9060394 | 14.2 |
| (OD04340) | | | |
| Kidney Ca, Nuclear grade | 1.3 | Gastric Cancer 064005 | 8.1 |
| 3 (OD04348) | | | |

TABLE EE
Panel 4D

| | Rel. Exp. (%) | Rel. Exp. (%) | | Rel. Exp. (%) | Rel. Exp. (%) |
|---|---|---|---|---|---|
| | Ag3055, Run | Ag3061, Run | | Ag3055, Run | Ag3061, Run |
| Tissue Name | 164317260 | 164528813 | Tissue Name | 164317260 | 164528813 |
| Secondary Th1 act | 4.7 | 4.7 | HUVEC IL-1beta | 1.3 | 0.4 |
| Secondary Th2 act | 9.2 | 7.7 | HUVEC IFN | 7.0 | 7.3 |
| | | | gamma | | |
| Secondary Tr1 act | 6.3 | 8.3 | HUVEC TNF alpha + | 2.9 | 5.4 |
| | | | IFN gamma | | |
| Secondary Th1 rest | 37.1 | 26.1 | HUVEC TNF alpha + | 3.2 | 3.1 |
| | | | IL4 | | |
| Secondary Th2 rest | 27.9 | 29.1 | HUVEC IL-11 | 4.7 | 6.0 |
| Secondary Tr1 rest | 42.3 | 36.6 | Lung Microvascular | 3.2 | 8.1 |
| | | | EC none | | |
| Primary Th1 act | 12.1 | 5.9 | Lung Microvascular | 5.0 | 7.2 |
| | | | EC TNFalpha + IL- | | |
| | | | 1beta | | |
| Primary Th2 act | 11.8 | 6.1 | Microvascular | 6.0 | 4.9 |
| | | | Dermal EC none | | |
| Primary Tr1 act | 7.9 | 4.9 | Microsvasular | 1.6 | 3.8 |
| | | | Dermal EC | | |
| | | | TNFalpha + IL- | | |
| | | | 1beta | | |
| Primary Th1 rest | 52.1 | 46.7 | Bronchial | 1.4 | 6.0 |
| | | | epithelium | | |
| | | | TNFalpha + | | |
| | | | IL1beta | | |
| Primary Th2 rest | 64.6 | 42.6 | Small airway | 0.5 | 0.5 |
| | | | epithelium none | | |
| Primary Tr1 rest | 32.5 | 48.0 | Small airway | 4.5 | 3.5 |
| | | | epithelium | | |
| | | | TNFalpha + IL- | | |
| | | | 1beta | | |
| CD45RA CD4 | 3.6 | 3.8 | Coronery artery | 4.1 | 4.6 |
| lymphocyte act | | | SMC rest | | |
| CD45RO CD4 | 8.1 | 11.8 | Coronery artery | 2.2 | 2.2 |
| lymphocyte act | | | SMC TNFalpha + | | |
| | | | IL-1beta | | |
| CD8 lymphocyte act | 7.1 | 11.3 | Astrocytes rest | 5.6 | 9.0 |
| Secondary CD8 | 5.0 | 10.0 | Astrocytes | 4.2 | 3.8 |
| lymphocyte rest | | | TNFalpha + IL- | | |
| | | | 1beta | | |
| Secondary CD8 | 9.9 | 9.3 | KU-812 (Basophil) | 5.0 | 5.6 |
| lymphocyte act | | | rest | | |
| CD4 lymphocyte | 20.0 | 12.8 | KU-812 (Basophil) | 5.5 | 4.1 |
| none | | | PMA/ionomycin | | |
| 2ry | 81.2 | 100.0 | CCD1106 | 3.8 | 4.8 |
| Th1/Th2/Tr1_anti- | | | (Keratinocytes) | | |
| CD95 CH11 | | | none | | |
| LAK cells rest | 11.3 | 7.9 | CCD1106 | 0.0 | 0.0 |
| | | | (Keratinocytes) | | |
| | | | TNFalpha + IL- | | |
| | | | 1beta | | |
| LAK cells IL-2 | 13.5 | 12.7 | Liver cirrhosis | 7.0 | 8.5 |
| LAK cells IL-2 + IL- | 5.1 | 3.2 | Lupus kidney | 5.1 | 5.9 |
| 12 | | | | | |
| LAK cells IL- | 9.0 | 7.7 | NCI-H292 none | 21.6 | 21.5 |
| 2 + IFN gamma | | | | | |
| LAK cells IL-2 + IL- | 8.7 | 6.3 | NCI-H292 IL-4 | 15.9 | 10.4 |
| 18 | | | | | |
| LAK cells | 2.8 | 3.3 | NCI-H292 IL-9 | 24.0 | 19.2 |
| PMA/ionomycin | | | | | |
| NK Cells IL-2 rest | 8.8 | 17.0 | NCI-H292 IL-13 | 11.1 | 16.5 |
| Two Way MLR 3 | 12.1 | 10.4 | NCI-H292 IFN | 8.5 | 5.3 |
| day | | | gamma | | |
| Two Way MLR 5 | 1.5 | 3.8 | HPAEC none | 2.1 | 2.4 |
| day | | | | | |
| Two Way MLR 7 | 1.6 | 6.0 | HPAEC TNF alpha + | 1.2 | 1.6 |
| day | | | IL-1 beta | | |
| PBMC rest | 7.3 | 9.5 | Lung fibroblast | 6.1 | 11.4 |
| | | | none | | |
| PBMC PWM | 10.6 | 18.6 | Lung fibroblast | 4.9 | 2.5 |
| | | | TNF alpha + IL-1 | | |
| | | | beta | | |
| PBMC PHA-L | 11.3 | 7.7 | Lung fibroblast IL- | 7.7 | 8.4 |
| | | | 4 | | |
| Ramos (B cell) none | 2.4 | 1.6 | Lung fibroblast IL- | 5.1 | 7.2 |
| | | | 9 | | |
| Ramos (B cell) | 9.2 | 6.6 | Lung fibroblast IL- | 1.6 | 4.8 |
| ionomycin | | | 13 | | |
| B lymphocytes | 17.9 | 13.9 | Lung fibroblast IFN | 10.1 | 9.0 |
| PWM | | | gamma | | |
| B lymphocytes | 75.8 | 66.0 | Dermal fibroblast | 3.2 | 6.0 |
| CD40L and IL-4 | | | CCD1070 rest | | |
| EOL-1 dbcAMP | 7.2 | 7.1 | Dermal fibroblast | 27.7 | 34.2 |
| | | | CCD1070 TNF | | |
| | | | alpha | | |
| EOL-1 dbcAMP | 70.7 | 60.3 | Dermal fibroblast | 3.5 | 0.6 |
| PMA/ionomycin | | | CCD1070 IL-1 beta | | |
| Dendritic cells none | 11.7 | 14.1 | Dermal fibroblast | 4.6 | 2.9 |
| | | | IFN gamma | | |
| Dendritic cells LPS | 2.6 | 3.8 | Dermal fibroblast | 7.7 | 6.2 |
| | | | IL-4 | | |
| Dendritic cells anti- | 18.6 | 17.6 | IBD Colitis 2 | 5.1 | 3.0 |
| CD40 | | | | | |
| Monocytes rest | 3.4 | 11.3 | IBD Crohn's | 4.9 | 6.3 |
| Monocytes LPS | 1.4 | 0.2 | Colon | 33.0 | 43.5 |
| Macrophages rest | 20.0 | 19.6 | Lung | 6.9 | 10.1 |
| Macrophages LPS | 1.2 | 1.6 | Thymus | 85.9 | 73.7 |
| HUVEC none | 2.9 | 2.6 | Kidney | 100.0 | 61.1 |
| HUVEC starved | 5.6 | 5.8 | | | |

TABLE EF
Panel CNS_1

| | Rel. Exp. (%) Ag3055, Run | | Rel. Exp. (%) Ag3055, Run |
|---|---|---|---|
| Tissue Name | 171694541 | Tissue Name | 171694541 |
| BA4 Control | 10.2 | BA17 PSP | 1.2 |
| BA4 Control2 | 23.8 | BA17 PSP2 | 5.4 |
| BA4 | 4.9 | Sub Nigra Control | 11.7 |
| Alzheimer's2 | | | |
| BA4 Parkinson's | 35.8 | Sub Nigra Control2 | 6.2 |
| BA4 Parkinson's2 | 60.3 | Sub Nigra | 8.0 |
| | | Alzheimer's2 | |
| BA4 Huntington's | 18.7 | Sub Nigra | 42.3 |
| | | Parkinson's2 | |
| BA4 | 16.2 | Sub Nigra | 23.0 |
| Huntington's2 | | Huntington's | |
| BA4 PSP | 3.8 | Sub Nigra | 33.4 |
| | | Huntington's2 | |
| BA4 PSP2 | 13.2 | Sub Nigra PSP2 | 2.0 |
| BA4 Depression | 26.6 | Sub Nigra Depression | 4.6 |
| BA4 Depression2 | 22.1 | Sub Nigra | 9.3 |
| | | Depression2 | |
| BA7 Control | 25.9 | Glob Palladus Control | 0.8 |
| BA7 Control2 | 11.2 | Glob Palladus | 0.0 |
| | | Control2 | |
| BA7 | 24.7 | Glob Palladus | 2.4 |
| Alzheimer's2 | | Alzheimer's | |
| BA7 Parkinson's | 33.2 | Glob Palladus | 9.7 |
| | | Alzheimer's2 | |
| BA7 Parkinson's2 | 41.8 | Glob Palladus | 1.0 |
| | | Parkinson's | |
| BA7 Huntington's | 32.3 | Glob Palladus | 14.3 |
| | | Parkinson's2 | |
| BA7 | 100.0 | Glob Palladus PSP | 3.7 |
| Huntington's2 | | | |
| BA7 PSP | 40.1 | Glob Palladus PSP2 | 6.2 |
| BA7 PSP2 | 18.7 | Glob Palladus | 10.1 |
| | | Depression | |
| BA7 Depression | 4.6 | Temp Pole Control | 7.1 |
| BA9 Control | 12.7 | Temp Pole Control2 | 35.4 |
| BA9 Control2 | 42.6 | Temp Pole | 6.2 |
| | | Alzheimer's | |
| BA9 Alzheimer's | 4.8 | Temp Pole | 7.6 |
| | | Alzheimer's2 | |
| BA9 | 27.2 | Temp Pole Parkinson's | 40.3 |
| Alzheimer's2 | | | |
| BA9 Parkinson's | 39.2 | Temp Pole | 33.2 |
| | | Parkinson's2 | |
| BA9 Parkinson's2 | 40.3 | Temp Pole | 18.2 |
| | | Huntington's | |
| BA9 Huntington's | 16.6 | Temp Pole PSP | 2.2 |
| BA9 | 31.0 | Temp Pole PSP2 | 0.0 |
| Huntington's2 | | | |
| BA9 PSP | 8.8 | Temp Pole | 17.1 |
| | | Depression2 | |
| BA9 PSP2 | 2.5 | Cing Gyr Control | 34.4 |
| BA9 Depression | 12.9 | Cing Gyr Control2 | 18.8 |
| BA9 Depression2 | 14.9 | Cing Gyr Alzheimer's | 11.5 |
| BA17 Control | 30.4 | Cing Gyr | 18.7 |
| | | Alzheimer's2 | |
| BA17 Control2 | 14.4 | Cing Gyr Parkinson's | 40.6 |
| BA17 | 18.3 | Cing Gyr Parkinson's2 | 48.6 |
| Alzheimer's2 | | | |
| BA17 Parkinson's | 52.9 | Cing Gyr Huntington's | 20.4 |
| BA17 | 42.3 | Cing Gyr | 32.8 |
| Parkinson's2 | | Huntington's2 | |
| BA17 | 17.0 | Cing Gyr PSP | 2.2 |
| Huntington's | | | |
| BA17 | 35.8 | Cing Gyr PSP2 | 2.9 |
| Huntington's2 | | | |
| BA17 Depression | 34.2 | Cing Gyr Depression | 17.8 |
| BA17 | 55.9 | Cing Gyr Depression2 | 10.2 |
| Depression2 | | | |

CNS_neurodegeneration_v1.0 Summary: Ag3055 Results from two experiments with the NOV7 gene are not included because the amp plot indicates that there were experimental difficulties with this run.

Panel 1.3D Summary: Ag3055/3061 The NOV7 gene was run on 2 independent panels with excellent concordance between the panels. There is a low level of expression in most of the tissues in this panel, with the highest expression in a breast cancer cell line T47D (CTs=29). Therefore, expression of this gene may be used as a diagnostic marker for breast cancer. Furthermore, inhibition of this gene product using antibodies or amall molecule inhibitors may be useful for the treatment of breast cancer.

Among metabolic tissues, this gene has low levels of expression in pancreas, thyroid, pituitary, adrenal, adult and fetal heart, adult and fetal skeletal muscle, adult and fetal liver, and adipose. Therefore, this putative esterase may be a small molecule target for the treatment of metabolic and endocrine disease, including the thyroidopathies, Types 1 and 2 diabetes, and obesity.

In addition, this gene exhibits moderate expression throughout the brain, indicating a functional role in the CNS. Neuropathy target esterase is a known mediator of neuronal degeneration, a common feature of diseases such as Alzheimer's disease, Parkinson's disease, Huntington's disease, and other diseases involving neurodegeneration. Therefore, agents that enhance the function of this gene product may have utility as therapeutics in the treatment of these diseases.

### REFERENCES

Lush M J, Li Y, Read D J, Willis A C, Glynn P. Neuropathy target esterase and a homologous Drosophila neurodegeneration-associated mutant protein contain a novel domain conserved from bacteria to man. Biochem J May 15, 1998;332 (Pt 1):1-4

The N-terminal amino acid sequences of proteolytic fragments of neuropathy target esterase (NTE), covalently labelled on its active-site serine by a biotinylated organophosphorus ester, were determined and used to deduce the location of this serine residue and to initiate cloning of its cDNA. A putative NTE clone, isolated from a human foetal brain cDNA library, encoded a 1327 residue polypeptide with no homology to any known serine esterases or proteases. The active-site serine of NTE (Ser-966) lay in the centre of a predicted hydrophobic helix within a 200-amino-acid C-terminal domain with marked similarity to conceptual proteins in bacteria, yeast and nematodes; these proteins may comprise a novel family of potential serine hydrolases. The Swiss Cheese protein which, when mutated, leads to widespread cell death in Drosophila brain [Kretzschmar, Hasan, Sharma, Heisenberg and Benzer (1997) J. Neurosci. 17, 7425-7432], was strikingly homologous to NTE, suggesting that genetically altered NTE may be involved in human neurodegenerative disease.(NTE), covalently labelled on its active-site serine by a biotinylated organophosphorus ester, were determined and used to deduce the location of this serine residue and to initiate cloning of its cDNA. A putative NTE clone, isolated from a human foetal brain cDNA library, encoded a 1327 residue polypeptide with no homology to any known serine esterases or proteases. The active-site serine of NTE (Ser-966) lay in the centre of a predicted hydrophobic helix within a 200-amino-acid C-terminal domain with marked similarity to conceptual proteins in bacteria, yeast and nematodes; these proteins may comprise a novel family of potential serine hydrolases. The Swiss Cheese protein which, when mutated, leads to widespread cell death in Drosophila brain [Kretzschmar, Hasan, Sharma, Heisenberg and Benzer (1997) J. Neurosci. 17, 7425-7432], was strikingly homologous to NTE, suggesting that genetically altered NTE may be involved in human neurodegenerative disease.

Panel 2.2 Summary: Ag3055 Significant expression of the NOV7 gene is restricted to kidney cancer samples. The highest level of expression is seen in a kidney cancer sample (CT=32.72). In addition, there is slightly higher expression in two kidney cancers compared to the normal adjacent tissue. Thus, this gene could be used as a diagnostic marker for the presence of kidney cancer. Furthermore, antibodies or small molecule inhibitors could potentially be used for the treatment of kidney cancer.

Panel 4D Summary: Ag3055/Ag3061 Two experiments produce results that are in excellent agreement. This gene, a neuropathy target esterase homolog is expressed at a moderate level in several preparations of activated and resting T lymphocytes, activated B lymphocytes, the eosinophil cell line Eol-1, cytokine-activated lung and skin fibroblasts and lung mucoepidermoid NCI-H292 cells (CT range 29-33). This widespread expression in both cell lines and tissues involved in the autoimmune response suggests that small molecules that antagonize the NOV7 gene product may reduce or eliminate the symptoms in patients with autoimmune and inflammatory diseases, including Crohn's disease, ulcerative colitis, multiple sclerosis, chronic obstructive pulmonary disease, asthma, emphysema, rheumatoid arthritis, lupus erythematosus, or psoriasis.

Panel CNS_{—}1 Summary: Ag3055 The results of this experiment confirm expression of the NOV7 gene in the brain. Please see Panel 1.3D for discussion of utility of this gene in the central nervous system.

F. NOV8—CG57119-01: Acid-Sensitive Potassium Channel Protein Task

Expression of the NOV8 gene was assessed using the primer-probe sets Ag241 and Ag3074, described in Tables FA and FB. Results of the RTQ-PCR runs are shown in Tables FC, FD, FE, FF and FG.

TABLE FA

Probe Name Ag241

| | | | | |
|---|---|---|---|---|
| Primers | Sequences | Length | Start Position | SEQ ID NO: |

| | | | | |
|---|---|---|---|---|
| Forward | 5′-cagggtcgaatctggaatgg-3′ | 20 | 141 | 485 |
| Probe | TET-5′-tctggcttcagctatcagggcaccc-3′-TAMRA | 25 | 111 | 486 |
| Reverse | 5′-cccgtcatccgtttccaat-3′ | 19 | 83 | 487 |

TABLE FR

Probe Name Ag3074

| | | | | |
|---|---|---|---|---|
| Primers | Sequences | Length | Start Position | SEQ ID NO: |

| | | | | |
|---|---|---|---|---|
| Forward | 5′-gtccttctacttcgccatc-3′ | 20 | 611 | 488 |
| Probe | TET-5′-tcatcactaccatcgagtacggccac-3-TAMRA | 26 | 581 | 489 |
| Reverse | 5′-acatgcagaagaccttgcc-3′ | 19 | 541 | 490 |

TABLE FC
Panel 1.3D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag241, | Ag241, | Ag3074, | | Ag241, | Ag241, | Ag3074, |
| | Run | Run | Run | Tissue | Run | Run | Run |
| Tissue Name | 155695586 | 163728044 | 163724451 | Name | 155695586 | 163728044 | 163724451 |
| Liver | 0.3 | 1.7 | 6.9 | Kidney | 1.0 | 0.0 | 0.4 |
| adenocarcinoma | | | | (fetal) | | | |
| Pancreas | 0.5 | 1.2 | 0.6 | Renal ca. | 0.0 | 0.0 | 0.0 |
| | | | | 786-0 | | | |
| Pancreatic ca. | 0.4 | 0.0 | 0.0 | Renal ca. | 0.4 | 0.0 | 0.0 |
| CAPAN2 | | | | A498 | | | |
| Adrenal gland | 1.4 | 1.3 | 0.6 | Renal ca. | 0.1 | 0.4 | 2.1 |
| | | | | RXF 393 | | | |
| Thyroid | 4.1 | 4.4 | 19.8 | Renal ca. | 14.0 | 23.5 | 28.7 |
| | | | | ACHN | | | |
| Salivary gland | 1.0 | 0.1 | 1.3 | Renal ca. | 0.0 | 0.0 | 0.0 |
| | | | | UO-31 | | | |
| Pituitary gland | 2.9 | 2.0 | 0.6 | Renal ca. | 0.0 | 0.0 | 0.0 |
| | | | | TK-10 | | | |
| Brain (fetal) | 0.3 | 0.0 | 0.0 | Liver | 0.0 | 0.0 | 0.0 |
| Brain (whole) | 0.1 | 0.3 | 0.9 | Liver (fetal) | 0.0 | 0.0 | 0.0 |
| Brain (amygdala) | 0.4 | 0.1 | 0.0 | Liver ca. | 0.0 | 0.0 | 0.0 |
| | | | | (hepatoblast) | | | |
| | | | | HepG2 | | | |
| Brain | 0.0 | 0.0 | 0.0 | Lung | 3.8 | 0.7 | 1.4 |
| (cerebellum) | | | | | | | |
| Brain | 1.3 | 0.0 | 0.0 | Lung (fetal) | 0.0 | 0.4 | 0.3 |
| (hippocampus) | | | | | | | |
| Brain (substantia | 0.2 | 0.0 | 0.4 | Lung ca. | 0.2 | 0.0 | 0.9 |
| nigra) | | | | (small cell) | | | |
| | | | | LX-1 | | | |
| Brain (thalamus) | 0.2 | 0.0 | 1.2 | Lung ca. | 2.7 | 1.4 | 0.3 |
| | | | | (small cell) | | | |
| | | | | NCI-H69 | | | |
| Cerebral Cortex | 0.1 | 2.3 | 1.5 | Lung ca. | 0.0 | 0.0 | 0.0 |
| | | | | (s. cell var.) | | | |
| | | | | SHP-77 | | | |
| Spinal cord | 0.6 | 0.9 | 0.7 | Lung ca. | 0.2 | 0.6 | 0.0 |
| | | | | (large | | | |
| | | | | cell) NCI- | | | |
| | | | | H460 | | | |
| glio/astro U87- | 0.2 | 0.4 | 0.6 | Lung ca. | 4.1 | 1.2 | 2.5 |
| MG | | | | (non-sm. | | | |
| | | | | cell) A549 | | | |
| glio/astro U-118- | 7.8 | 2.9 | 4.3 | Lung ca. | 39.0 | 50.0 | 45.1 |
| MG | | | | (non-s. cell) | | | |
| | | | | NCI-H23 | | | |
| astrocytoma | 0.0 | 0.0 | 0.0 | Lung ca. | 0.2 | 0.0 | 0.0 |
| SW1783 | | | | (non-s. cell) | | | |
| | | | | HOP-62 | | | |
| neuro*; met SK- | 0.6 | 0.0 | 0.0 | Lung ca. | 0.2 | 0.4 | 0.3 |
| N-AS | | | | (non-s. cl) | | | |
| | | | | NCI-H522 | | | |
| astrocytoma SF- | 0.1 | 0.6 | 0.0 | Lung ca | 0.8 | 1.1 | 0.0 |
| 539 | | | | (squam.) | | | |
| | | | | SW 900 | | | |
| astrocytoma | 2.3 | 0.6 | 0.0 | Lung ca. | 0.1 | 0.9 | 1.0 |
| SNB-75 | | | | (squam.) | | | |
| | | | | NCI-H596 | | | |
| glioma SNB-19 | 0.0 | 0.5 | 0.7 | Mammary | 3.0 | 1.6 | 0.4 |
| | | | | gland | | | |
| glioma U251 | 0.0 | 0.3 | 0.0 | Breast ca.* | 43.5 | 73.2 | 43.8 |
| | | | | (pl. ef) MCF- | | | |
| | | | | 7 | | | |
| glioma SF-295 | 0.2 | 0.4 | 0.6 | Breast ca.* | 0.0 | 0.0 | 0.0 |
| | | | | (pl. ef) | | | |
| | | | | MDA-MB- | | | |
| | | | | 231 | | | |
| Heart (fetal) | 0.6 | 0.0 | 0.3 | Breast ca.* | 9.2 | 9.2 | 24.0 |
| | | | | (pl. ef) T47D | | | |
| Heart | 0.2 | 1.5 | 6.5 | Breast ca. | 0.8 | 0.0 | 0.4 |
| | | | | BT-549 | | | |
| Skeletal muscle | 1.4 | 2.6 | 1.7 | Breast ca. | 0.1 | 0.0 | 0.0 |
| (fetal) | | | | MDA-N | | | |
| Skeletal muscle | 0.5 | 0.4 | 0.0 | Ovary | 3.3 | 6.5 | 11.0 |
| Bone marrow | 0.0 | 0.0 | 0.0 | Ovarian ca. | 11.6 | 18.7 | 19.1 |
| | | | | OVCAR-3 | | | |
| Thymus | 0.0 | 0.8 | 2.2 | Ovarian ca. | 10.4 | 5.7 | 9.0 |
| | | | | OVCAR-4 | | | |
| Spleen | 0.0 | 0.0 | 0.7 | Ovarian ca. | 1.3 | 2.3 | 3.4 |
| | | | | OVCAR-5 | | | |
| Lymph node | 0.4 | 0.0 | 0.0 | Ovarian ca. | 7.9 | 9.3 | 12.8 |
| | | | | OVCAR-8 | | | |
| Colorectal | 0.9 | 0.4 | 1.0 | Ovarian ca. | 0.1 | 0.0 | 0.0 |
| | | | | IGROV-1 | | | |
| Stomach | 1.5 | 0.7 | 0.7 | Ovarian ca.* | 3.8 | 2.8 | 5.1 |
| | | | | (ascites) SK- | | | |
| | | | | OV-3 | | | |
| Small intestine | 0.4 | 0.3 | 0.3 | Uterus | 2.1 | 1.3 | 3.9 |
| Colon ca. SW480 | 1.3 | 0.6 | 0.6 | Placenta | 0.0 | 0.7 | 0.5 |
| Colon ca.* | 0.2 | 1.3 | 0.0 | Prostate | 0.6 | 0.4 | 7.2 |
| SW620 (SW480 | | | | | | | |
| met) | | | | | | | |
| Colon ca. HT29 | 0.0 | 0.0 | 0.0 | Prostate ca.* | 17.3 | 21.3 | 33.9 |
| | | | | (bone | | | |
| | | | | met) PC-3 | | | |
| Colon ca. HCT- | 0.0 | 0.0 | 0.0 | Testis | 7.1 | 3.2 | 9.2 |
| 116 | | | | | | | |
| Colon ca. CaCo- | 3.0 | 4.7 | 2.4 | Melanoma | 0.0 | 0.0 | 0.0 |
| 2 | | | | Hs688(A).T | | | |
| Colon ca. | 1.5 | 3.0 | 5.4 | Melanoma* | 0.0 | 0.0 | 1.4 |
| tissue (ODO3866) | | | | (met) | | | |
| | | | | Hs688(B).T | | | |
| Colon ca. HCC- | 2.4 | 1.6 | 1.0 | Melanoma | 0.0 | 0.5 | 0.0 |
| 2998 | | | | UACC-62 | | | |
| Gastric ca.* | 100.0 | 100.0 | 100.0 | Melanoma | 0.0 | 0.0 | 0.4 |
| (liver met) NCI- | | | | M14 | | | |
| N87 | | | | | | | |
| Bladder | 3.0 | 5.8 | 12.9 | Melanoma | 0.0 | 0.0 | 0.0 |
| | | | | LOX IMVI | | | |
| Trachea | 4.4 | 3.1 | 9.0 | Melanoma* | 0.0 | 0.0 | 0.0 |
| | | | | (met) SK- | | | |
| | | | | MEL-5 | | | |
| Kidney | 0.1 | 0.4 | 1.6 | Adipose | 2.3 | 2.2 | 2.6 |

TABLE FD
Panel 2D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag241, | Ag241, | Ag3074, | | Ag241, | Ag241, | Ag3074, |
| | Run | Run | Run | Tissue | Run | Run | Run |
| Tissue Name | 155695603 | 163578011 | 163578433 | Name | 155695603 | 163578011 | 163578433 |
| Normal | 3.7 | 6.9 | 2.1 | Kidney | 1.3 | 1.2 | 2.3 |
| Colon | | | | Margin | | | |
| | | | | 8120608 | | | |
| CC Well to | 6.0 | 6.3 | 1.9 | Kidney | 0.0 | 0.0 | 0.0 |
| Mod Diff | | | | Cancer | | | |
| (ODO3866) | | | | 8120613 | | | |
| CC Margin | 0.7 | 0.0 | 0.9 | Kidney | 3.2 | 0.0 | 1.7 |
| (ODO3866) | | | | Margin | | | |
| | | | | 8120614 | | | |
| CC Gr.2 | 0.0 | 0.0 | 0.0 | Kidney | 5.1 | 3.3 | 2.9 |
| rectosigmoid | | | | Cancer | | | |
| (ODO3868) | | | | 9010320 | | | |
| CC Margin | 0.0 | 0.0 | 0.0 | Kidney | 6.2 | 2.6 | 3.6 |
| (ODO3868) | | | | Margin | | | |
| | | | | 9010321 | | | |
| CC Mod Diff | 0.0 | 0.0 | 0.0 | Normal | 6.3 | 10.8 | 8.8 |
| (ODO3920) | | | | Uterus | | | |
| CC Margin | 0.0 | 0.8 | 0.0 | Uterus | 3.5 | 1.1 | 3.5 |
| (ODO3920) | | | | Cancer | | | |
| | | | | 064011 | | | |
| CC Gr.2 | 18.7 | 14.1 | 7.1 | Normal | 18.3 | 10.5 | 5.8 |
| ascend colon | | | | Thyroid | | | |
| (ODO3921) | | | | | | | |
| CC Margin | 1.1 | 0.6 | 1.3 | Thyroid | 21.8 | 23.0 | 15.6 |
| (ODO3921) | | | | Cancer | | | |
| | | | | 064010 | | | |
| CC from | 0.4 | 3.8 | 0.6 | Thyroid | 15.8 | 15.4 | 12.1 |
| Partial | | | | Cancer | | | |
| Hepatectomy | | | | A302152 | | | |
| (ODO4309) | | | | | | | |
| Mets | | | | | | | |
| Liver Margin | 0.0 | 0.0 | 0.0 | Thyroid | 6.0 | 8.0 | 5.5 |
| (ODO4309) | | | | Margin | | | |
| | | | | A302153 | | | |
| Colon mets to | 1.0 | 3.5 | 0.0 | Normal | 8.5 | 12.7 | 4.8 |
| lung | | | | Breast | | | |
| (OD04451- | | | | | | | |
| 01) | | | | | | | |
| Lung Margin | 2.4 | 1.6 | 0.3 | Breast | 71.7 | 79.6 | 43.2 |
| (OD04451- | | | | Cancer | | | |
| 02) | | | | (OD04566) | | | |
| Normal | 3.1 | 15.8 | 10.8 | Breast | 88.3 | 55.9 | 100.0 |
| Prostate | | | | Cancer | | | |
| 6546-1 | | | | (OD04590- | | | |
| | | | | 01) | | | |
| Prostate | 0.7 | 2.0 | 2.1 | Breast | 66.9 | 59.5 | 80.1 |
| Cancer | | | | Cancer | | | |
| (OD04410) | | | | Mets | | | |
| | | | | (OD04590- | | | |
| | | | | 03) | | | |
| Prostate | 3.5 | 2.2 | 1.2 | Breast | 100.0 | 100.0 | 82.9 |
| Margin | | | | Cancer | | | |
| (OD04410) | | | | Metastasis | | | |
| | | | | (OD04655- | | | |
| | | | | 05) | | | |
| Prostate | 1.6 | 1.4 | 1.4 | Breast | 6.0 | 7.9 | 2.8 |
| Cancer | | | | Cancer | | | |
| (OD04720- | | | | 064006 | | | |
| 01) | | | | | | | |
| Prostate | 5.3 | 5.8 | 6.5 | Breast | 13.1 | 4.1 | 3.6 |
| Margin | | | | Cancer | | | |
| (OD04720- | | | | 1024 | | | |
| 02) | | | | | | | |
| Normal Lung | 2.3 | 3.1 | 2.0 | Breast | 90.8 | 80.7 | 69.7 |
| 061010 | | | | Cancer | | | |
| | | | | 9100266 | | | |
| Lung Met to | 1.7 | 1.2 | 0.2 | Breast | 16.8 | 18.6 | 7.1 |
| Muscle | | | | Margin | | | |
| (ODO4286) | | | | 9100265 | | | |
| Muscle | 5.6 | 3.5 | 5.2 | Breast | 4.2 | 0.3 | 2.0 |
| Margin | | | | Cancer | | | |
| (ODO4286) | | | | A209073 | | | |
| Lung | 12.8 | 8.8 | 13.9 | Breast | 4.5 | 2.0 | 3.1 |
| Malignant | | | | Margin | | | |
| Cancer | | | | A209073 | | | |
| (OD03126) | | | | | | | |
| Lung Margin | 2.0 | 2.9 | 2.9 | Normal | 0.0 | 0.0 | 0.0 |
| (OD03126) | | | | Liver | | | |
| Lung Cancer | 3.1 | 2.0 | 2.7 | Liver | 0.0 | 0.0 | 0.0 |
| (OD04404) | | | | Cancer | | | |
| | | | | 064003 | | | |
| Lung Margin | 11.0 | 9.6 | 4.3 | Liver | 0.0 | 0.0 | 0.8 |
| (OD04404) | | | | Cancer | | | |
| | | | | 1025 | | | |
| Lung Cancer | 0.0 | 0.0 | 0.0 | Liver | 4.3 | 7.6 | 3.4 |
| (OD04565) | | | | Cancer | | | |
| | | | | 1026 | | | |
| Lung Margin | 2.4 | 1.7 | 0.5 | Liver | 0.0 | 0.0 | 0.4 |
| (OD04565) | | | | Cancer | | | |
| | | | | 6004-T | | | |
| Lung Cancer | 0.0 | 0.0 | 1.4 | Liver | 0.7 | 0.0 | 0.0 |
| (OD04237- | | | | Tissue | | | |
| 01) | | | | 6004-N | | | |
| Lung Margin | 5.4 | 5.6 | 2.7 | Liver | 2.6 | 7.4 | 3.5 |
| (OD04237- | | | | Cancer | | | |
| 02) | | | | 6005-T | | | |
| Ocular Mel | 3.1 | 0.9 | 0.7 | Liver | 0.5 | 0.6 | 0.6 |
| Met to Liver | | | | Tissue | | | |
| (ODO4310) | | | | 6005-N | | | |
| Liver Margin | 0.0 | 0.0 | 0.8 | Normal | 12.9 | 15.0 | 8.3 |
| (ODO4310) | | | | Bladder | | | |
| Melanoma | 12.9 | 12.9 | 13.9 | Bladder | 0.4 | 1.6 | 0.2 |
| Mets to Lung | | | | Cancer | | | |
| (OD04321) | | | | 1023 | | | |
| Lung Margin | 7.5 | 8.4 | 8.8 | Bladder | 0.7 | 2.9 | 0.0 |
| (OD04321) | | | | Cancer | | | |
| | | | | A302173 | | | |
| Normal | 2.3 | 0.0 | 1.1 | Bladder | 9.6 | 19.5 | 5.6 |
| Kidney | | | | Cancer | | | |
| | | | | (OD04718- | | | |
| | | | | 01) | | | |
| Kidney Ca, | 2.4 | 6.0 | 1.5 | Bladder | 5.3 | 10.2 | 6.5 |
| Nuclear grade | | | | Normal | | | |
| 2 (OD04338) | | | | Adjacent | | | |
| | | | | (OD04718- | | | |
| | | | | 03) | | | |
| Kidney | 2.3 | 2.9 | 1.5 | Normal | 5.3 | 5.7 | 9.4 |
| Margin | | | | Ovary | | | |
| (OD04338) | | | | | | | |
| Kidney Ca | 2.5 | 7.3 | 0.8 | Ovarian | 70.7 | 62.9 | 67.8 |
| Nuclear grade | | | | Cancer | | | |
| 1/2 | | | | 064008 | | | |
| (OD04339) | | | | | | | |
| Kidney | 1.6 | 3.9 | 1.4 | Ovarian | 9.9 | 4.8 | 5.3 |
| Margin | | | | Cancer | | | |
| (OD04339) | | | | (OD04768- | | | |
| | | | | 07) | | | |
| Kidney Ca, | 0.9 | 0.0 | 0.2 | Ovary | 1.2 | 6.2 | 4.0 |
| Clear cell | | | | Margin | | | |
| type | | | | (OD04768- | | | |
| (OD04340) | | | | 08) | | | |
| Kidney | 4.6 | 3.7 | 1.7 | Normal | 2.1 | 1.9 | 0.9 |
| Margin | | | | Stomach | | | |
| (OD04340) | | | | | | | |
| Kidney Ca, | 0.0 | 0.2 | 1.1 | Gastric | 2.5 | 2.9 | 0.4 |
| Nuclear grade | | | | Cancer | | | |
| 3 (OD04348) | | | | 9060358 | | | |
| Kidney | 2.2 | 0.7 | 2.4 | Stomach | 0.6 | 2.4 | 1.2 |
| Margin | | | | Margin | | | |
| (OD04348) | | | | 9060359 | | | |
| Kidney | 0.0 | 0.7 | 0.1 | Gastric | 8.4 | 6.3 | 2.5 |
| Cancer | | | | Cancer | | | |
| (OD04622- | | | | 9060395 | | | |
| 01) | | | | | | | |
| Kidney | 5.6 | 5.8 | 4.7 | Stomach | 4.0 | 2.5 | 0.9 |
| Margin | | | | Margin | | | |
| (OD04622- | | | | 9060394 | | | |
| 03) | | | | | | | |
| Kidney | 27.0 | 16.3 | 9.0 | Gastric | 0.4 | 1.9 | 0.7 |
| Cancer | | | | Cancer | | | |
| (OD04450- | | | | 9060397 | | | |
| 01) | | | | | | | |
| Kidney | 0.0 | 1.0 | 1.4 | Stomach | 1.5 | 1.2 | 1.0 |
| Margin | | | | Margin | | | |
| (OD04450- | | | | 9060396 | | | |
| 03) | | | | | | | |
| Kidney | 0.6 | 1.7 | 2.2 | Gastric | 4.5 | 3.0 | 2.6 |
| Cancer | | | | Cancer | | | |
| 8120607 | | | | 064005 | | | |

TABLE FE
Panel 3D

| | Rel. Exp. (%) | | Rel. Exp. (%) |
|---|---|---|---|
| | Ag241, Run | | Ag241, Run |
| Tissue Name | 165022800 | Tissue Name | 165022800 |
| Daoy- Medulloblastoma | 0.8 | Ca Ski- Cervical epidermoid | 0.0 |
| | | carcinoma (metastasis) | |
| TE671- Medulloblastoma | 2.1 | ES-2- Ovarian clear cell | 0.0 |
| | | carcinoma | |
| D283 Med- | 0.0 | Ramos- Stimulated with | 0.0 |
| Medulloblastoma | | PMA/ionomycin 6h | |
| PFSK-1- Primitive | 0.0 | Ramos- Stimulated with | 0.0 |
| Neuroectodermal | | PMA/ionomycin 14h | |
| XF-498- CNS | 0.0 | MEG-01- Chronic myelogenous | 0.0 |
| | | leukemia (megokaryoblast) | |
| SNB-78- Glioma | 0.0 | Raji- Burkitt's lymphoma | 0.0 |
| SF-268- Glioblastoma | 0.0 | Daudi- Burkitt's lymphoma | 0.0 |
| T98G- Glioblastoma | 0.0 | U266- B-cell plasmacytoma | 6.8 |
| SK-N-SH- Neuroblastoma | 1.8 | CA46- Burkitt's lymphoma | 0.0 |
| (metastasis) | | | |
| SF-295- Glioblastoma | 0.0 | RL- non-Hodgkin's B-cell | 0.0 |
| | | lymphoma | |
| Cerebellum | 1.1 | JM1- pre-B-cell lymphoma | 0.0 |
| Cerebellum | 0.0 | Jurkat- T cell leukemia | 0.0 |
| NCI-H292- | 6.8 | TF-1- Erythroleukemia | 0.0 |
| Mucoepidermoid lung | | | |
| carcinoma | | | |
| DMS-114- Small cell lung | 0.8 | HUT 78- T-cell lymphoma | 0.0 |
| cancer | | | |
| DMS-79- Small cell lung | 9.5 | U937- Histiocytic lymphoma | 0.0 |
| cancer | | | |
| NCI-H146- Small cell lung | 0.7 | KU-812- Myelogenous leukemia | 0.0 |
| cancer | | | |
| NCI-H526- Small cell lung | 4.0 | 769-P- Clear cell renal | 0.0 |
| cancer | | carcinoma | |
| NCI-N417- Small cell lung | 0.8 | Caki-2- Clear cell renal | 0.0 |
| cancer | | carcinoma | |
| NCI-H82- Small cell lung | 0.0 | SW 839- Clear cell renal | 0.0 |
| cancer | | carcinoma | |
| NCI-H157- Squamous cell | 0.0 | G401- Wilms' tumor | 28.1 |
| lung cancer (metastasis) | | | |
| NCI-H1155- Large cell | 0.0 | Hs766T- Pancreatic carcinoma | 0.4 |
| lung cancer | | (LN metastasis) | |
| NCI-H1299- Large cell | 0.0 | CAPAN-1- Pancreatic | 2.0 |
| lung cancer | | adenocarcinoma (liver | |
| | | metastasis) | |
| NCI-H727- Lung carcinoid | 1.0 | SU86.86- Pancreatic carcinoma | 0.7 |
| | | (liver metastasis) | |
| NCI-UMC-11- Lung | 0.0 | BxPC-3- Pancreatic | 1.7 |
| carcinoid | | adenocarcinoma | |
| LX-1- Small cell lung | 0.0 | HPAC- Pancreatic | 1.4 |
| cancer | | adenocarcinoma | |
| Colo-205- Colon cancer | 0.0 | MIA PaCa-2- Pancreatic | 4.6 |
| | | carcinoma | |
| KM12- Colon cancer | 0.0 | CFPAC-1- Pancreatic ductal | 0.0 |
| | | adenocarcinoma | |
| KM20L2- Colon cancer | 1.8 | PANC-1- Pancreatic epithelioid | 15.7 |
| | | ductal carcinoma | |
| NCI-H716- Colon cancer | 0.0 | T24- Bladder carcinma | 0.0 |
| | | (transitional cell) | |
| SW-48- Colon | 0.0 | 5637- Bladder carcinoma | 2.3 |
| adenocarcinoma | | | |
| SW1116- Colon | 0.0 | HT-1197- Bladder carcinoma | 0.0 |
| adenocarcinoma | | | |
| LS 174T- Colon | 14.4 | UM-UC-3- Bladder carcinma | 0.0 |
| adenocarcinoma | | (transitional cell) | |
| SW-948- Colon | 0.0 | A204- Rhabdomyosarcoma | 0.0 |
| adenocarcinoma | | | |
| SW-480- Colon | 0.6 | HT-1080- Fibrosarcoma | 0.0 |
| adenocarcinoma | | | |
| NCI-SNU-5- Gastric | 1.4 | MG-63- Osteosarcoma | 0.0 |
| carcinoma | | | |
| KATO III- Gastric | 0.0 | SK-LMS-1- Leiomyosarcoma | 7.6 |
| carcinoma | | (vulva) | |
| NCI-SNU-16- Gastric | 0.0 | SJRH30- Rhabdomyosarcoma | 0.0 |
| carcinoma | | (met to bone marrow) | |
| NCI-SNU-1- Gastric | 0.0 | A431- Epidermoid carcinoma | 0.0 |
| carcinoma | | | |
| RF-1- Gastric | 0.0 | WM266-4- Melanoma | 19.1 |
| adenocarcinoma | | | |
| RF-48- Gastric | 0.0 | DU 145- Prostate carcinoma | 0.0 |
| adenocarcinoma | | (brain metastasis) | |
| MKN-45- Gastric | 18.2 | MDA-MB-468- Breast | 0.5 |
| carcinoma | | adenocarcinoma | |
| NCI-N87- Gastric | 12.2 | SCC-4- Squamous cell | 0.0 |
| carcinoma | | carcinoma of tongue | |
| OVCAR-5- Ovarian | 0.0 | SCC-9- Squamous cell | 0.0 |
| carcinoma | | carcinoma of tongue | |
| RL95-2- Uterine carcinoma | 0.0 | SCC-15- Squamous cell | 0.0 |
| | | carcinoma of tongue | |
| HelaS3- Cervical | 100.0 | CAL 27- Squamous cell | 2.0 |
| adenocarcinoma | | carcinoma of tongue | |

TABLE FF
Panel 4.1D

| | Rel. Exp. (%) Ag3074, | | Rel. Exp. (%) Ag3074, |
|---|---|---|---|
| Tissue Name | Run 248389309 | Tissue Name | Run 248389309 |
| Secondary Th1 act | 0.0 | HUVEC IL-1beta | 0.0 |
| Secondary Th2 act | 1.6 | HUVEC IFN gamma | 0.0 |
| Secondary Tr1 act | 3.3 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 5.2 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 5.6 | Coronery artery SMC rest | 0.0 |
| lymphocyte act | | | |
| CD45RO CD4 | 4.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + IL- | 5.8 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 0.0 |
| LAK cells IL-2 + IL-12 | 0.0 | NCI-H292 none | 13.7 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 17.4 |
| LAK cells | 0.0 | NCI-H292 IL-13 | 20.7 |
| PMA/ionomycin | | | |
| NK Cells IL-2 rest | 0.0 | NCI-H292 IFN gamma | 13.0 |
| Two Way MLR 3 day | 0.0 | HPAEC none | 0.0 |
| Two Way MLR 5 day | 0.0 | HPAEC TNF alpha + IL-1 | 0.0 |
| | | beta | |
| Two Way MLR 7 day | 0.0 | Lung fibroblast none | 0.0 |
| PBMC rest | 0.0 | Lung fibroblast TNF alpha + | 0.0 |
| | | IL-1 beta | |
| PBMC PWM | 0.0 | Lung fibroblast IL-4 | 0.0 |
| PBMC PHA-L | 3.4 | Lung fibroblast IL-9 | 0.0 |
| Ramos (B cell) none | 0.0 | Lung fibroblast IL-13 | 0.0 |
| Ramos (B cell) | 0.0 | Lung fibroblast IFN gamma | 0.0 |
| ionomycin | | | |
| B lymphocytes PWM | 0.0 | Dermal fibroblast CCD1070 | 4.2 |
| | | rest | |
| B lymphocytes CD40L | 1.0 | Dermal fibroblast CCD1070 | 0.0 |
| and IL-4 | | TNF alpha | |
| EOL-1 dbcAMP | 0.0 | Dermal fibroblast CCD1070 | 1.0 |
| | | IL-1 beta | |
| EOL-1 dbcAMP | 0.0 | Dermal fibroblast IFN | 100.0 |
| PMA/ionomycin | | gamma | |
| Dendritic cells none | 0.0 | Dermal fibroblast IL-4 | 78.5 |
| Dendritic cells LPS | 0.0 | Dermal Fibroblasts rest | 59.9 |
| Dendritic cells anti- | 0.0 | Neutrophils TNFa + LPS | 0.0 |
| CD40 | | | |
| Monocytes rest | 0.0 | Neutrophils rest | 0.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

TABLE FG
Panel 4D

| | Rel. Exp. (%) | Rel. Exp. (%) | | Rel. Exp. (%) | Rel. Exp. (%) |
|---|---|---|---|---|---|
| | Ag241, Run | Ag3074, Run | | Ag241, Run | Ag3074, Run |
| Tissue Name | 165010380 | 162598884 | Tissue Name | 165010380 | 162598884 |
| Secondary Th1 act | 5.6 | 0.0 | HUVEC IL-1beta | 0.0 | 0.0 |
| Secondary Th2 act | 1.7 | 0.0 | HUVEC IFN | 0.0 | 0.0 |
| | | | gamma | | |
| Secondary Tr1 act | 0.0 | 0.0 | HUVEC TNF alpha + | 0.0 | 0.0 |
| | | | IFN gamma | | |
| Secondary Th1 rest | 0.0 | 0.0 | HUVEC TNF alpha + | 0.0 | 0.0 |
| | | | IL4 | | |
| Secondary Th2 rest | 0.0 | 0.0 | HUVEC IL-11 | 0.0 | 0.0 |
| Secondary Tr1 rest | 0.0 | 0.0 | Lung Microvascular | 0.0 | 0.0 |
| | | | EC none | | |
| Primary Th1 act | 40.9 | 33.7 | Lung Microvascular | 0.0 | 0.0 |
| | | | EC TNFalpha + IL- | | |
| | | | 1beta | | |
| Primary Th2 act | 2.0 | 0.0 | Microvascular | 0.0 | 0.0 |
| | | | Dermal EC none | | |
| Primary Tr1 act | 6.7 | 0.0 | Microsvasular | 0.0 | 0.0 |
| | | | Dermal EC | | |
| | | | TNFalpha + IL- | | |
| | | | 1beta | | |
| Primary Th1 rest | 1.5 | 0.0 | Bronchial | 1.4 | 0.0 |
| | | | epithelium | | |
| | | | TNFalpha + | | |
| | | | IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium | | |
| | | | TNFalpha + IL- | | |
| | | | 1beta | | |
| CD45RA CD4 | 2.7 | 0.0 | Coronery artery | 6.9 | 0.0 |
| lymphocyte act | | | SMC rest | | |
| CD45RO CD4 | 5.7 | 0.0 | Coronery artery | 0.0 | 0.0 |
| lymphocyte act | | | SMC TNFalpha + | | |
| | | | IL-1beta | | |
| CD8 lymphocyte act | 0.0 | 2.6 | Astrocytes rest | 0.0 | 0.0 |
| Secondary CD8 | 1.6 | 11.2 | Astrocytes | 1.3 | 12.9 |
| lymphocyte rest | | | TNFalpha + IL- | | |
| | | | 1beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 0.0 | 0.0 | CCD1106 | 0.0 | 0.0 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNFalpha + IL- | | |
| | | | 1beta | | |
| LAK cells IL-2 | 1.7 | 0.0 | Liver cirrhosis | 10.2 | 2.7 |
| LAK cells IL-2 + IL- | 1.7 | 0.0 | Lupus kidney | 0.2 | 3.1 |
| 12 | | | | | |
| LAK cells IL- | 0.0 | 0.0 | NCI-H292 none | 19.6 | 9.9 |
| 2 + IFN gamma | | | | | |
| LAK cells IL-2 + IL- | 0.0 | 1.6 | NCI-H292 IL-4 | 25.3 | 3.5 |
| 18 | | | | | |
| LAK cells | 0.2 | 0.0 | NCI-H292 IL-9 | 74.7 | 31.6 |
| PMA/ionomycin | | | | | |
| NK Cells IL-2 rest | 0.0 | 0.0 | NCI-H292 IL-13 | 21.0 | 12.8 |
| Two Way MLR 3 | 0.0 | 0.0 | NCI-H292 IFN | 23.2 | 29.1 |
| day | | | gamma | | |
| Two Way MLR 5 | 0.0 | 0.0 | HPAEC none | 0.0 | 0.0 |
| day | | | | | |
| Two Way MLR 7 | 0.0 | 0.0 | HPAEC TNF alpha + | 0.0 | 0.0 |
| day | | | IL-1 beta | | |
| PBMC rest | 0.0 | 0.0 | Lung fibroblast | 0.0 | 0.0 |
| | | | none | | |
| PBMC PWM | 14.5 | 2.2 | Lung fibroblast | 0.0 | 0.0 |
| | | | TNF alpha + IL-1 | | |
| | | | beta | | |
| PBMC PHA-L | 4.1 | 2.0 | Lung fibroblast IL- | 0.0 | 0.0 |
| | | | 4 | | |
| Ramos (B cell) none | 0.0 | 0.0 | Lung fibroblast IL- | 0.0 | 0.0 |
| | | | 9 | | |
| Ramos (B cell) | 0.0 | 0.0 | Lung fibroblast IL- | 0.0 | 2.6 |
| ionomycin | | | 13 | | |
| B lymphocytes | 27.2 | 4.2 | Lung fibroblast IFN | 0.0 | 0.0 |
| PWM | | | gamma | | |
| B lymphocytes | 3.1 | 0.0 | Dermal fibroblast | 5.5 | 3.2 |
| CD40L and IL-4 | | | CCD1070 rest | | |
| EOL-1 dbcAMP | 0.0 | 0.0 | Dermal fibroblast | 1.6 | 0.0 |
| | | | CCD1070 TNF | | |
| | | | alpha | | |
| EOL-1 dbcAMP | 0.0 | 0.0 | Dermal fibroblast | 2.6 | 0.0 |
| PMA/ionomycin | | | CCD1070 IL-1 beta | | |
| Dendritic cells none | 0.0 | 0.0 | Dermal fibroblast | 100.0 | 100.0 |
| | | | IFN gamma | | |
| Dendritic cells LPS | 0.0 | 2.8 | Dermal fibroblast | 87.7 | 61.1 |
| | | | IL-4 | | |
| Dendritic cells anti- | 0.0 | 0.0 | IBD Colitis 2 | 0.0 | 0.0 |
| CD40 | | | | | |
| Monocytes rest | 0.0 | 0.0 | IBD Crohn's | 0.0 | 0.0 |
| Monocytes LPS | 1.6 | 0.0 | Colon | 52.5 | 3.0 |
| Macrophages rest | 0.0 | 0.0 | Lung | 43.5 | 25.3 |
| Macrophages LPS | 0.0 | 0.0 | Thymus | 9.7 | 3.7 |
| HUVEC none | 0.0 | 0.0 | Kidney | 4.7 | 4.9 |
| HUVEC starved | 0.0 | 0.0 | | | |

Panel 1 Summary: Ag241 Expression of the NOV8 gene is low/undetectable in all samples on this panel (CTs>35). (Data not shown.) The amp plot indicates that there is a high probability of a probe failure.

Panel 1.3D Summary: Ag241/Ag3074 Three experiments with two different probe and primer sets produce results that are in very good agreement. Expression of the NOV8 gene in this panel is most prominent in cancer cell lines, with highest expression in a gastric cancer cell line (CTs=28). Significant levels of expression are also seen in cell lines derived from prostate cancer, ovarian cancer, breast cancer, lung cancer, and renal cancer. Thus, the therapeutic inhibition of this gene activity, through the use of small molecule drugs or antibodies, might be of utility in the treatment of the above listed cancer types. In addition, expression of this gene could be used as a diagnostic marker for cancer.

Among metabolic tissues, the NOV8 gene has a low level of expression in adrenal, pituitary, heart and adipose. Thus, this gene product may be a small molecule target for the treatment of metabolic and endocrine disease, including the adrenalopathies, obesity and Type 2 diabetes.

Results from one experiment with the Ag241 show low/undetectable levels of expression in all the samples on this panel (CTs>35). (Data not shown.)

### REFERENCES

Maingret F, Patel A J, Lesage F, Lazdunski M, Honore E. Lysophospholipids open the two-pore domain mechano-gated K(+) channels TREK-1 and TRAAK. J. Biol. Chem. Apr. 7, 2000;275(14):10128-33.

The two-pore (2P) domain K(+) channels TREK-1 and TRAAK are opened by membrane stretch as well as arachidonic acid (AA) (Patel, A. J., Honore, E., Maingret, F., Lesage, F., Fink, M., Duprat, F., and Lazdunski, M. (1998) EMBO J. 17, 4283-4290; Maingret, F., Patel, A. J., Lesage, F., Lazdunski, M., and Honore, E. (1999) J. Biol. Chem. 274, 26691-26696; Maingret, F., Fosset, M., Lesage, F., Lazdunski, M., and Honore, E. (1999) J. Biol. Chem. 274, 1381-1387. We demonstrate that lysophospholipids (LPs) and platelet-activating factor also produce large specific and reversible activations of TREK-I and TRAAK. LPs activation is a function of the size of the polar head and length of the acyl chain but is independent of the charge of the molecule. Bath application of lysophosphatidylcholine (LPC) immediately opens TREK-1 and TRAAK in the cell-attached patch configuration. In excised patches, LPC activation is lost, whereas AA still produces maximal opening. The carboxyl-terminal region of TREK-1, but not the amino terminus and the extracellular loop M1P1, is critically required for LPC activation. LPC activation is indirect and may possibly involve a cytosolic factor, whereas AA directly interacts with either the channel proteins or the bilayer and mimics stretch. Opening of TREK-1 and TRAAK by fatty acids and LPs may be an important switch in the regulation of synaptic function and may also play a protective role during ischemia and inflammation.

PMID: 10744694

Panel 2D Summary: Ag241/Ag3041 The expression of the NOV8 gene was assessed in three independent runs with good concordance between the runs. This gene is expressed at a higher level in colon, thyroid, breast and bladder cancer samples compared to normal adjacent tissues. Hence this gene can be used as a diagnostic marker for these cancers and inhibition of the gene product using antibodies or small molecule drugs can be used for the treatment of these cancers.

Panel 3D Summary: Ag241 The expression of the NOV8 gene was assessed in one run. This gene is expressed in in several cell lines including melanoma, gastric cancer, kidney cancer, cervical cancer and lung cancer cell lines. Thus, the therapeutic inhibition of this gene activity, through the use of small molecule drugs or antibodies, might be of utility in the treatment of the above listed cancer types.

Panels 4D/4.1D Summary: Ag241/Ag3074 Two experiments with two different probe and primer sets show highest expression of the NOV8 gene in dermal fibroblasts treated with IFN-gamma (CTs=30-33). Significant expression is also seen in dermal fibroblasts treated with IL-4. This expression suggests that the protein encoded by this gene may be involved in skin disorders, such as psoriasis. Significant levels of expression are also seen in both treated and untreated samples derived from the mucoepidermoid pulmonary cell line NCI-H292. This expression profile suggests that the gene product may also be involved in inflammatory processes that affect the lung. Therefore, therapeutic modulation of the expression or function of the protein encoded by this gene may be effective in the treatment of asthma, allergies, emphysema and COPD.

G. NOV10—CG56860-01: Prostaglandin Omega-Hydroxylase Like Gene

Expression of the NOV10 gene was assessed using the primer-probe set Ag3038, described in Table GA. Results of the RTQ-PCR runs are shown in Table GB.

TABLE GA

Probe Name Ag3038

| | | | | |
|---|---|---|---|---|
| Primers | Sequences | Length | Start Position | SEQ ID NO: |

| | | | | |
|---|---|---|---|---|
| Forward | 5′-acagactcccagatggtgtct-3′ | 21 | 36 | 491 |
| Probe | TET-5′ctcctccaaggagcctcactgctgag-3′TAMRA | 26 | 62 | 492 |
| Reverse | 5′-ggctgccttcaatagtaacaga-3′ | 22 | 94 | 493 |

TABLE GB
Panel 4D

| | Rel. Exp. (%) Ag3038, | | Rel. Exp. (%) Ag3038, |
|---|---|---|---|
| Tissue Name | Run 164528701 | Tissue Name | Run 164528701 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1 beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN | 0.0 |
| | | gamma | |
| Dendritic cells LPS | 0.0 | Dermal fibroblast IL-4 | 4.2 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 1.7 |
| Macrophages rest | 0.0 | Lung | 9.5 |
| Macrophages LPS | 0.0 | Thymus | 16.2 |
| HUVEC none | 0.0 | Kidney | 6.8 |
| HUVEC starved | 0.0 | | |

CNS_neurodegeneration_v1.0 Summary: Ag3038 Expression of the NOV10 gene is low/undetectable in all samples on this panel (CTs>35). (Data not shown.)

Panel 13D Summary: Ag3038 Expression of the NOV10 gene is low/undetectable in all samples on this panel (CTs>35). (Data not shown.)

Panel 4D Summary: Ag3038 Significant expression of the NOV10 gene is restricted to a liver cirrhosis sample (CT=34). Therefore, antibodies or small molecule therapeutics designed with this gene product may reduce or inhibit fibrosis that occurs in liver cirrhosis. In addition, expression of this gene could also be used for the diagnosis of liver cirrhosis.

H. NOV11—CG57024-01: Myeloid Upregulated Protein

Expression of the NOV11 gene was assessed using the primer-probe set Ag3064, described in Table HA. Results of the RTQ-PCR runs are shown in Table HB.

TABLE HA

Probe Name Ag3064

| | | | | |
|---|---|---|---|---|
| Primers | Sequences | Length | Start Position | SEQ ID NO: |

| | | | | |
|---|---|---|---|---|
| Forward | 5′-caagtacggtgagcccaaa-3′ | 19 | 920 | 494 |
| Probe | TET-5′-ctgtccctgggacaccagctggt-3′-TAMRA | 23 | 965 | 495 |
| Reverse | 5′-caggttgacgtaggtgaagatg-3′ | 22 | 994 | 496 |

TABLE HB
Panel 4D

| | Rel. Exp. (%) | | Rel. Exp. (%) |
|---|---|---|---|
| | Ag3064, | | Ag3064, |
| Tissue Name | Run 164317426 | Tissue Name | Run 164317426 |
| Secondary Th1 act | 0.0 | HUVEC IL-1beta | 0.0 |
| Secondary Th2 act | 0.0 | HUVEC IFN gamma | 3.3 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 4.5 | Lung Microvascular EC | 6.6 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal | 0.0 |
| | | TNFalpha + IL-1beta | |
| Primary Th1 rest | 0.0 | Bronchial epithelium | 0.0 |
| | | TNFalpha + IL-1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 5.7 |
| Secondary CD8 | 5.3 | Astrocytes TNFalpha + IL- | 4.6 |
| lymphocyte rest | | 1beta | |
| Secondary CD8 | 0.0 | KU-812 (Basophil) rest | 6.3 |
| lymphocyte act | | | |
| CD4 lymphocyte none | 0.0 | KU-812 (Basophil) | 7.2 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 29.9 |
| LAK cells IL-2 + IL-12 | 0.0 | Lupus kidney | 4.1 |
| LAK cells IL-2 + IFN | 0.0 | NCI-H292 none | 5.6 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 10.0 |
| Two Way MLR 5 day | 0.0 | HPAEC none | 0.0 |
| Two Way MLR 7 day | 0.0 | HPAEC TNF alpha + IL-1 | 13.7 |
| | | beta | |
| PBMC rest | 0.0 | Lung fibroblast none | 0.0 |
| PBMC PWM | 6.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 7.2 |
| Ramos (B cell) | 4.0 | Lung fibroblast IL-13 | 0.0 |
| ionomycin | | | |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 6.3 | Dermal fibroblast CCD1070 | 0.0 |
| | | TNF alpha | |
| EOL-1 dbcAMP | 5.1 | Dermal fibroblast CCD1070 | 6.0 |
| PMA/ionomycin | | IL-1 beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 5.6 | Colon | 100.0 |
| Macrophages rest | 0.0 | Lung | 81.8 |
| Macrophages LPS | 0.0 | Thymus | 0.0 |
| HUVEC none | 0.0 | | 0.0 |
| HUVEC starved | 0.0 | | |

Panel 1.3D Summary: Ag3064 Results from one experiment with the NOV11 gene are not included. The amp plot indicates that there were experimental difficulties with this run.

Panel 4D Summary: Ag3064 Expression of the NOV11 gene is expressed at low levels in normal colon and lung (CTs=34.5), and may be useful as a marker for colon and lung tissue.

I. NOV12—CG57083-01: Testicular Serine Protease Like

Expression of the NOV112 gene was assessed using the primer-probe set Ag563, described in Table IA. Results of the RTQ-PCR runs are shown in Tables IB, IC, ID, and IE.

TABLE IA

Probe Name Ag563

| | | | | |
|---|---|---|---|---|
| Primers | Sequences | Length | Start Position | SEQ ID NO: |

| | | | | |
|---|---|---|---|---|
| Forward | 5′-gaagatgtctgtgcaccggat-3′ | 21 | 546 | 497 |
| Probe | TET-5′-cacccatccagactttgagaagctccac-3′-TAMRA | 28 | 570 | 498 |
| Reverse | 5′-catggcaatgtcactcccaa-3′ | 20 | 602 | 499 |

TABLE IB
General_screening_panel_v1.4

| | Rel. Exp. (%) | | Rel. Exp. (%) |
|---|---|---|---|
| | Ag563, | | Ag563, |
| Tissue Name | Run 219923406 | Tissue Name | Run 219923406 |
| Adipose | 0.0 | Renal ca. TK-10 | 7.7 |
| Melanoma* | 0.0 | Bladder | 0.0 |
| Hs688(A).T | | | |
| Melanoma* | 1.8 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 3.4 |
| LOXIMVI | | | |
| Melanoma* SK-MEL- | 0.0 | Colon ca. SW480 | 0.0 |
| 5 | | | |
| Squamous cell | 0.0 | Colon ca.* (SW480 met) | 0.0 |
| carcinoma SCC-4 | | SW620 | |
| Testis Pool | 0.0 | Colon ca. HT29 | 0.0 |
| Prostate ca.* (bone | 0.0 | Colon ca. HCT-116 | 3.3 |
| met) PC-3 | | | |
| Prostate Pool | 0.0 | Colon ca. CaCo-2 | 0.0 |
| Placenta | 1.8 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 3.1 | Colon ca. SW-48 | 0.0 |
| Ovarian ca. OVCAR-4 | 0.0 | Colon Pool | 1.6 |
| Ovarian ca. OVCAR-5 | 0.0 | Small Intestine Pool | 6.3 |
| Ovarian ca. IGROV-1 | 0.0 | Stomach Pool | 1.3 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 1.9 |
| Breast ca. MDA-MB- | 0.0 | Lymph Node Pool | 0.0 |
| 231 | | | |
| Breast ca. BT 549 | 1.8 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 3.1 | Thymus Pool | 1.7 |
| Trachea | 0.0 | CNS cancer (glio/astro) | 0.0 |
| | | U87-MG | |
| Lung | 2.2 | CNS cancer (glio/astro) | 2.0 |
| | | U-118-MG | |
| Fetal Lung | 3.1 | CNS cancer (neuro; met) | 0.0 |
| | | SK-N-AS | |
| Lung ca. NCI-N417 | 0.0 | CNS cancer (astro) SF- | 0.0 |
| | | 539 | |
| Lung ca. LX-1 | 0.0 | CNS cancer (astro) SNB- | 1.6 |
| | | 75 | |
| Lung ca. NCI-H146 | 0.0 | CNS cancer (glio) SNB- | 0.0 |
| | | 19 | |
| Lung ca. SHP-77 | 100.0 | CNS cancer (glio) SF-295 | 1.7 |
| Lung ca. A549 | 0.0 | Brain (Amygdala) Pool | 0.0 |
| Lung ca. NCI-H526 | 0.0 | Brain (cerebellum) | 2.3 |
| Lung ca. NCI-H23 | 3.7 | Brain (fetal) | 0.0 |
| Lung ca. NCI-H460 | 0.0 | Brain (Hippocampus) | 0.0 |
| | | Pool | |
| Lung ca. HOP-62 | 0.0 | Cerebral Cortex Pool | 0.0 |
| Lung ca. NCI-H522 | 0.0 | Brain (Substantia nigra) | 0.0 |
| | | Pool | |
| Liver | 0.0 | Brain (Thalamus) Pool | 0.0 |
| Fetal Liver | 4.0 | Brain (whole) | 0.7 |
| Liver ca. HepG2 | 18.3 | Spinal Cord Pool | 0.0 |
| Kidney Pool | 1.8 | Adrenal Gland | 0.0 |
| Fetal Kidney | 0.0 | Pituitary gland Pool | 1.5 |
| Renal ca. 786-0 | 0.0 | Salivary Gland | 0.0 |
| Renal ca. A498 | 7.0 | Thyroid (female) | 0.0 |
| Renal ca. ACHN | 5.4 | Pancreatic ca. CAPAN2 | 0.0 |
| Renal ca. UO-31 | 1.5 | Pancreas Pool | 0.0 |

TABLE IC
Panel 1.1

| | Rel. Exp. (%) | | Rel. Exp. (%) |
|---|---|---|---|
| | Ag563, | | Ag563, |
| Tissue Name | Run 109491882 | Tissue Name | Run 109491882 |
| Adrenal gland | 0.0 | Renal ca. UO-31 | 5.7 |
| Bladder | 1.6 | Renal ca. RXF 393 | 0.0 |
| Brain (amygdala) | 0.5 | Liver | 0.0 |
| Brain (cerebellum) | 15.8 | Liver (fetal) | 0.1 |
| Brain (hippocampus) | 6.2 | Liver ca. (hepatoblast) | 79.0 |
| | | HepG2 | |
| Brain (substantia nigra) | 4.2 | Lung | 0.0 |
| Brain (thalamus) | 2.3 | Lung (fetal) | 0.0 |
| Cerebral Cortex | 0.9 | Lung ca. (non-s. cell) | 2.1 |
| | | HOP-62 | |
| Brain (fetal) | 6.1 | Lung ca. (large | 1.7 |
| | | cell) NCI-H460 | |
| Brain (whole) | 10.5 | Lung ca. (non-s. cell) | 3.6 |
| | | NCI-H23 | |
| glio/astro U-118-MG | 2.5 | Lung ca. (non-s. cl) | 0.2 |
| | | NCI-H522 | |
| astrocytoma SF-539 | 0.1 | Lung ca. (non-sm. cell) | 7.3 |
| | | A549 | |
| astrocytoma SNB-75 | 2.2 | Lung ca. (s. cell var.) | 100.0 |
| | | SHP-77 | |
| astrocytoma SW1783 | 0.5 | Lung ca. (small cell) | 2.1 |
| | | LX-1 | |
| glioma U251 | 1.3 | Lung ca. (small cell) | 59.5 |
| | | NCI-H69 | |
| glioma SF-295 | 0.1 | Lung ca. (squam.) SW | 2.8 |
| | | 900 | |
| glioma SNB-19 | 3.8 | Lung ca. (squam.) | 18.6 |
| | | NCI-H596 | |
| glio/astro U87-MG | 2.0 | Lymph node | 0.0 |
| neuro*; met SK-N-AS | 1.5 | Spleen | 0.0 |
| Mammary gland | 0.0 | Thymus | 13.9 |
| Breast ca. BT-549 | 4.5 | Ovary | 0.0 |
| Breast ca. MDA-N | 4.7 | Ovarian ca. IGROV-1 | 9.8 |
| Breast ca.* (pl. ef) T47D | 6.8 | Ovarian ca. OVCAR-3 | 0.3 |
| Breast ca.* (pl. ef) MCF- | 1.6 | Ovarian ca. OVCAR-4 | 0.0 |
| 7 | | | |
| Breast ca.* (pl. ef) MDA- | 0.0 | Ovarian ca. OVCAR-5 | 27.5 |
| MB-231 | | | |
| Small intestine | 0.1 | Ovarian ca. OVCAR-8 | 3.1 |
| Colorectal | 0.2 | Ovarian ca.* (ascites) | 4.5 |
| | | SK-OV-3 | |
| Colon ca. HT29 | 4.9 | Pancreas | 0.2 |
| Colon ca. CaCo-2 | 0.0 | Pancreatic ca. CAPAN2 | 0.0 |
| Colon ca. HCT-15 | 13.8 | Pituitary gland | 0.4 |
| Colon ca. HCT-116 | 0.1 | Placenta | 0.0 |
| Colon ca. HCC-2998 | 4.7 | Prostate | 0.0 |
| Colon ca. SW480 | 0.0 | Prostate ca.* (bone | 0.0 |
| | | met) PC-3 | |
| Colon ca.* SW620 | 0.0 | Salivary gland | 0.0 |
| (SW480 met) | | | |
| Stomach | 0.2 | Trachea | 0.0 |
| Gastric ca. (liver met) | 5.2 | Spinal cord | 0.2 |
| NCI-N87 | | | |
| Heart | 0.1 | Testis | 35.6 |
| Skeletal muscle (Fetal) | 0.0 | Thyroid | 0.2 |
| Skeletal muscle | 1.1 | Uterus | 0.2 |
| Endothelial cells | 0.0 | Melanoma M14 | 11.4 |
| Heart (Fetal) | 0.0 | Melanoma LOX IMVI | 0.4 |
| Kidney | 0.0 | Melanoma UACC-62 | 0.0 |
| Kidney (fetal) | 1.9 | Melanoma SK-MEL- | 0.1 |
| | | 28 | |
| Renal ca. 786-0 | 1.2 | Melanoma* (met) SK- | 0.0 |
| | | MEL-5 | |
| Renal ca. A498 | 4.1 | Melanoma Hs688(A).T | 0.0 |
| Renal ca. ACHN | 2.9 | Melanoma* (met) | 1.8 |
| | | Hs688(B).T | |
| Renal ca. TK-10 | 6.3 | | |

TABLE ID
Panel 1.2

| | Rel. Exp. (%) | | Rel. Exp. (%) |
|---|---|---|---|
| | Ag563, | | Ag563, |
| Tissue Name | Run 117053190 | Tissue Name | Run 117053190 |
| Endothelial cells | 0.0 | Renal ca. 786-0 | 0.0 |
| Heart (Fetal) | 0.0 | Renal ca. A498 | 0.9 |
| Pancreas | 0.0 | Renal ca. RXF 393 | 0.0 |
| Pancreatic ca. CAPAN2 | 0.0 | Renal ca. ACHN | 0.0 |
| Adrenal Gland | 0.0 | Renal ca. UO-31 | 0.0 |
| Thyroid | 0.0 | Renal ca. TK-10 | 0.0 |
| Salivary gland | 0.0 | Liver | 0.0 |
| Pituitary gland | 4.0 | Liver (fetal) | 0.0 |
| Brain (fetal) | 95.9 | Liver ca. (hepatoblast) | 0.9 |
| | | HepG2 | |
| Brain (whole) | 64.6 | Lung | 0.0 |
| Brain (amygdala) | 3.9 | Lung (fetal) | 0.0 |
| Brain (cerebellum) | 21.3 | Lung ca. (small cell) | 0.0 |
| | | LX-1 | |
| Brain (hippocampus) | 33.4 | Lung ca. (small cell) | 1.1 |
| | | NCI-H69 | |
| Brain (thalamus) | 49.0 | Lung ca. (s. cell var.) | 4.0 |
| | | SHP-77 | |
| Cerebral Cortex | 47.6 | Lung ca. (large | 0.0 |
| | | cell) NCI-H460 | |
| Spinal cord | 4.7 | Lung ca. (non-sm. cell) | 0.0 |
| | | A549 | |
| glio/astro U87-MG | 0.0 | Lung ca. (non-s. cell) | 2.6 |
| | | NCI-H23 | |
| glio/astro U-118-MG | 0.0 | Lung ca. (non-s. cell) | 0.0 |
| | | HOP-62 | |
| astrocytoma SW1783 | 0.0 | Lung ca. (non-s. cl) | 40.6 |
| | | NCI-H522 | |
| neuro*; met SK-N-AS | 0.7 | Lung ca. (squam.) SW | 0.0 |
| | | 900 | |
| astrocytoma SF-539 | 0.0 | Lung ca. (squam.) NCI- | 0.0 |
| | | H596 | |
| astrocytoma SNB-75 | 0.0 | Mammary gland | 0.0 |
| glioma SNB-19 | 0.0 | Breast ca.* (pl. ef) | 0.0 |
| | | MCF-7 | |
| glioma U251 | 0.0 | Breast ca.* (pl. ef) | 0.0 |
| | | MDA-MB-231 | |
| glioma SF-295 | 0.0 | Breast ca.* (pl. ef) | 0.0 |
| | | T47D | |
| Heart | 0.0 | Breast ca. BT-549 | 0.0 |
| Skeletal Muscle | 0.0 | Breast ca. MDA-N | 0.0 |
| Bone marrow | 0.0 | Ovary | 0.2 |
| Thymus | 0.0 | Ovarian ca. OVCAR-3 | 0.0 |
| Spleen | 0.0 | Ovarian ca. OVCAR-4 | 0.0 |
| Lymph node | 0.0 | Ovarian ca. OVCAR-5 | 2.7 |
| Colorectal Tissue | 0.0 | Ovarian ca. OVCAR-8 | 0.0 |
| Stomach | 0.0 | Ovarian ca. IGROV-1 | 0.0 |
| Small intestine | 0.0 | Ovarian ca. (ascites) | 0.0 |
| | | SK-OV-3 | |
| Colon ca. SW480 | 0.0 | Uterus | 0.0 |
| Colon ca.* SW620 | 0.0 | Placenta | 0.0 |
| (SW480 met) | | | |
| Colon ca. HT29 | 0.0 | Prostate | 0.0 |
| Colon ca. HCT-116 | 0.0 | Prostate ca.* (bone | 0.0 |
| | | met) PC-3 | |
| Colon ca. CaCo-2 | 0.0 | Testis | 100.0 |
| Colon ca. Tissue | 0.0 | Melanoma Hs688(A).T | 0.0 |
| (ODO3866) | | | |
| Colon ca. HCC-2998 | 0.8 | Melanoma* (met) | 0.0 |
| | | Hs688(B).T | |
| Gastric ca.* (liver met) | 0.0 | Melanoma UACC-62 | 0.0 |
| NCI-N87 | | | |
| Bladder | 0.0 | Melanoma M14 | 0.0 |
| Trachea | 0.0 | Melanoma LOX IMVI | 0.0 |
| Kidney | 0.0 | Melanoma* (met) SK- | 0.0 |
| | | MEL-5 | |
| Kidney (fetal) | 0.0 | | |

TABLE IE
Panel 4D

| | Rel. Exp. (%) | | Rel. Exp. (%) |
|---|---|---|---|
| | Ag563, | | Ag563, |
| Tissue Name | Run 138134496 | Tissue Name | Run 138134496 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 21.6 |
| Secondary Th2 rest | 11.0 | HUVEC IL-11 | 14.0 |
| Secondary Tr1 rest | 0.0 | Lung Microvascular EC none | 0.0 |
| Primary Th1 act | 10.6 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 10.4 |
| | | TNFalpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 11.1 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 11.1 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 35.6 | CCD1106 (Keratinocytes) | 0.0 |
| CD95CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 100.0 |
| LAK cells IL-2 + IL-12 | 0.0 | Lupus kidney | 13.7 |
| LAK cells IL-2 + IFN | 0.0 | NCI-H292 none | 11.8 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 36.9 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 12.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 9.7 |
| ionomycin | | | |
| B lymphocytes PWM | 10.2 | Lung fibroblast IFN gamma | 0.0 |
| B lymphocytes CD40L | 0.0 | Dermal fibroblast CCD1070 | 11.1 |
| and IL-4 | | rest | |
| EOL-1 dbcAMP | 0.0 | Dermal fibroblast CCD1070 | 0.0 |
| | | TNF alpha | |
| EOL-1 dbcAMP | 11.9 | Dermal fibroblast CCD1070 | 0.0 |
| PMA/ionomycin | | IL-1beta | |
| Dendritic cells none | 0.0 | Dermal fibroblast IFN | 0.0 |
| | | gamma | |
| Dendritic cells LPS | 0.0 | Dermal fibroblast IL-4 | 16.4 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 61.1 |
| Macrophages rest | 0.0 | Lung | 43.5 |
| Macrophages LPS | 0.0 | Thymus | 0.0 |
| HUVEC none | 0.0 | Kidney | 0.0 |
| HUVEC starved | 0.0 | | |

General_screening panel_v1.4 Summary: Ag563 Expression of the NOV12 gene is restricted to a sample derived from a lung cancer cell line (CT=32.6). Thus, expression of this gene could be used to differentiate between this sample and other samples on this panel and as a marker to detect the presence of lung cancer. Furthermore, therapeutic modulation of the expression or function of this gene may be effective in the treatment of lung cancer.

Panel 1.1 Summary: Ag563 Highest expression of the NOV112 gene is seen in a lung cancer cell line (CT=26.7). Significant expression is also seen in clusters of cell line samples derived from melanoma, liver cancer, ovarian cancer, renal cancer and colon cancer. Thus, expression of the NOV12 gene could be used to differentiate these samples from other samples on this panel and as a marker to detect the presence of these cancers. Furthermore, therapeutic modulation of the expression or function of this gene may be effective in the treatment of these cancers.

While expression of this gene is predominant among cancer cell line samples, significant expression is also seen in the testis and the brain. Expression in the testis indicates that this gene product may be involved in male fertility. Furthermore, expression in the brain indicates that this gene product may be involved in the normal homeostasis of this organ.

Panel 1.2 Summary: Ag563 Expression of the NOV12 gene in this panel is in agreement with the expression seen in the previous panels. Significant expression is seen in testis, a lung cancer cell line and the brain. Please see Panel 1.1 for discussion of utility of this gene in these tissues.

Panel 4D Summary: Ag563 Significant expression of the NOV12 gene is detected in a liver cirrhosis sample (CT=32.7). Furthermore, expression of this gene is not detected in normal liver in Panels 1.1 and 1.2, suggesting that its expression is unique to liver cirrhosis. Therefore, antibodies or small molecule therapeutics designed with the protein encoded by this gene could reduce or inhibit fibrosis that occurs in liver cirrhosis. In addition, expression of this gene could also be used for the diagnosis of liver cirrhosis.

Panel 5 Islet Summary: Ag563 Expression of the NOV12 gene is low/undetectable in all samples on this panel (CTs>35). (Data not shown.)

Panel CNS_{—}1 Summary: Ag563 Expression of the NOV12 gene is low/undetectable in all samples on this panel (CTs>35). (Data not shown.)

J. NOV17—CG57177-01: Carboxypeptidase B

Expression of NOV17 gene was assessed using the primer-probe set Ag4136, described in Table JA. Results of the RTQ-PCR runs are shown in Tables JB, JC and JD.

TABLE JA

Probe Name Ag4136

| | | | | |
|---|---|---|---|---|
| Primers | Sequences | Length | Start Position | SEQ ID NO: |

| | | | | |
|---|---|---|---|---|
| Forward | 5′-attgacttctggaagccagatt-3′ | 22 | 148 | 500 |
| Probe | TET-5′tgtcacacaaatcaaacctcacagtaca-3′-TAMRA | 128 | 171 | 501 |
| Reverse | 5′-cttctgctttaacacggaagtc-3′ | 22 | 202 | 502 |

TABLE JB
CNS_neurodegeneration_v1.0

| | Rel. Exp. (%) | | Rel. Exp. (%) |
|---|---|---|---|
| | Ag4136, | | Ag4136, |
| Tissue Name | Run 214961497 | Tissue Name | Run 214961497 |
| AD 1 Hippo | 24.7 | Control (Path) 3 | 0.0 |
| | | Temporal Ctx | |
| AD 2 Hippo | 61.1 | Control (Path) 4 | 16.0 |
| | | Temporal Ctx | |
| AD 3 Hippo | 0.0 | AD 1 Occipital Ctx | 0.0 |
| AD 4 Hippo | 9.1 | AD 2 Occipital Ctx | 0.0 |
| | | (Missing) | |
| AD 5 hippo | 6.0 | AD 3 Occipital Ctx | 0.0 |
| AD 6 Hippo | 34.9 | AD 4 Occipital Ctx | 45.7 |
| Control 2 Hippo | 17.0 | AD 5 Occipital Ctx | 25.7 |
| Control 4 Hippo | 43.8 | AD 6 Occipital Ctx | 16.6 |
| Control (Path) 3 Hippo | 0.0 | Control 1 Occipital Ctx | 0.0 |
| AD 1 Temporal Ctx | 0.0 | Control 2 Occipital Ctx | 43.5 |
| AD 2 Temporal Ctx | 76.8 | Control 3 Occipital Ctx | 12.5 |
| AD 3 Temporal Ctx | 0.0 | Control 4 Occipital Ctx | 32.8 |
| AD 4 Temporal Ctx | 22.2 | Control (Path) 1 | 100.0 |
| | | Occipital Ctx | |
| AD 5 Inf Temporal Ctx | 11.9 | Control (Path) 2 | 27.2 |
| | | Occipital Ctx | |
| AD 5 Sup Temporal Ctx | 41.2 | Control (Path) 3 | 0.0 |
| | | Occipital Ctx | |
| AD 6 Inf Temporal Ctx | 48.0 | Control (Path) 4 | 21.3 |
| | | Occipital Ctx | |
| AD 6 Sup Temporal Ctx | 0.0 | Control 1 Parietal Ctx | 0.0 |
| Control 1 Temporal Ctx | 0.0 | Control 2 Parietal Ctx | 5.5 |
| Control 2 Temporal Ctx | 44.8 | Control 3 Parietal Ctx | 35.6 |
| Control 3 Temporal Ctx | 14.8 | Control (Path) 1 Parietal Ctx | 37.1 |
| Control 4 Temporal Ctx | 32.8 | Control (Path) 2 Parietal Ctx | 11.0 |
| Control (Path) 1 | 36.3 | Control (Path) 3 Parietal Ctx | 0.0 |
| Temporal Ctx | | | |
| Control (Path) 2 | 22.4 | Control (Path) 4 Parietal Ctx | 0.0 |
| Temporal Ctx | | | |

TABLE JC
General_screening_panel_v1.4

| | Rel. Exp. (%) | | Rel. Exp. (%) |
|---|---|---|---|
| | Ag4136, | | Ag4136, |
| Tissue Name | Run 220967145 | Tissue Name | Run 220967145 |
| Adipose | 0.0 | Renal ca. TK-10 | 0.0 |
| Melanoma* | 0.0 | Bladder | 100.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK-MEL- | 0.0 | Colon ca. SW480 | 0.0 |
| 5 | | | |
| Squamous cell | 0.0 | Colon ca.* (SW480 met) | 0.0 |
| carcinoma SCC-4 | | SW620 | |
| Testis Pool | 0.0 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 0.0 |
| Ovarian ca. OVCAR-5 | 0.0 | Small Intestine Pool | 0.0 |
| Ovarian ca. IGROV-1 | 0.0 | Stomach Pool | 0.0 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB- | 0.0 | Lymph Node Pool | 0.0 |
| 231 | | | |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.2 |
| Breast Pool | 0.0 | Thymus Pool | 0.0 |
| Trachea | 0.0 | CNS cancer (glio/astro) | 0.0 |
| | | U87-MG | |
| Lung | 0.0 | CNS cancer (glio/astro) | 0.0 |
| | | U-118-MG | |
| Fetal Lung | 0.0 | CNS cancer (neuro; met) | 0.0 |
| | | SK-N-AS | |
| Lung ca. NCI-N417 | 0.0 | CNS cancer (astro) SF- | 0.0 |
| | | 539 | |
| Lung ca. LX-1 | 0.0 | CNS cancer (astro) SNB- | 0.0 |
| | | 75 | |
| Lung ca. NCI-H146 | 0.0 | CNS cancer (glio) SNB- | 0.0 |
| | | 19 | |
| Lung ca. SHP-77 | 0.0 | CNS cancer (glio) SF- | 0.0 |
| | | 295 | |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 0.0 | Brain (Substantia nigra) | 0.0 |
| | | Pool | |
| Liver | 0.0 | Brain (Thalamus) Pool | 0.0 |
| Fetal Liver | 0.3 | Brain (whole) | 0.0 |
| Liver ca. HepG2 | 0.0 | Spinal Cord Pool | 0.0 |
| Kidney Pool | 0.0 | Adrenal Gland | 0.5 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 45.7 |

TABLE JD
Panel 4.1D

| | Rel. Exp. (%) | | Rel. Exp. (%) |
|---|---|---|---|
| | Ag4136, | | Ag4136, |
| Tissue Name | Run 173118872 | Tissue Name | Run 173118872 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 1.8 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes | 0.5 |
| lymphocyte rest | | TNFalpha + IL-1beta | |
| Secondary CD8 | 0.0 | KU-812 (Basophil) rest | 27.9 |
| lymphocyte act | | | |
| CD4 lymphocyte none | 0.0 | KU-812 (Basophil) | 62.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.3 | CCD1106 (Keratinocytes) | 0.0 |
| | | TNFalpha + IL-1beta | |
| LAK cells IL-2 | 0.5 | Liver cirrhosis | 0.0 |
| LAK cells IL-2 + IL-12 | 0.0 | NCI-H292 none | 0.0 |
| LAK cells IL-2 + IFN gamma | 0.0 | NCI-H292 IL-4 | 0.1 |
| LAK cells IL-2 + IL-18 | 0.0 | NCI-H292 IL-9 | 0.5 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.4 | HPAEC none | 0.0 |
| Two Way MLR 5 day | 0.0 | HPAEC TNF alpha + IL-1 | 0.5 |
| | | beta | |
| Two Way MLR 7 day | 0.0 | Lung fibroblast none | 2.0 |
| PBMC rest | 0.0 | Lung fibroblast TNF alpha + | 0.5 |
| | | IL-1 beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 1.1 |
| Ramos (B cell) | 0.0 | Lung fibroblast IFN gamma | 0.0 |
| ionomycin | | | |
| B lymphocytes PWM | 0.0 | Dermal fibroblast CCD1070 | 0.0 |
| | | rest | |
| B lymphocytes CD40L | 0.0 | Dermal fibroblast CCD1070 | 0.0 |
| and IL-4 | | TNF alpha | |
| EOL-1 dbcAMP | 0.0 | Dermal fibroblast CCD1070 | 0.0 |
| | | IL-1 beta | |
| EOL-1 dbcAMP | 0.0 | Dermal fibroblast IFN | 0.5 |
| PMA/ionomycin | | gamma | |
| Dendritic cells none | 0.0 | Dermal fibroblast IL-4 | 0.0 |
| Dendritic cells LPS | 2.0 | Dermal Fibroblasts rest | 1.3 |
| Dendritic cells anti- | 0.0 | Neutrophils TNFa + LPS | 0.4 |
| CD40 | | | |
| Monocytes rest | 0.0 | Neutrophils rest | 0.7 |
| Monocytes LPS | 0.0 | Colon | 0.9 |
| Macrophages rest | 0.0 | Lung | 4.5 |
| Macrophages LPS | 0.0 | Thymus | 19.1 |
| HUVEC none | 0.0 | Kidney | 100.0 |
| HUVEC starved | 0.0 | | |

CNS_neurodegeneration_v1.0 Summary: Ag4136 Expression levels of the NOV117 gene in the brain are very low. No disease association is evident by this panel. Carboxypeptidase B is, however, a known mediator of beta-amyloid clearance in the brain, and consequently plays an important role in Alzheimer's disease. Therefore, even low expression of the NOV17 gene may be sufficient to impart significant beta amyloid clearance, especially over time. Therefore, agents that augment the function of this gene product may have utility as therapeutics in the treatment of Alzheimer's disease.

### REFERENCES

Matsumoto A, Itoh K, Seki T, Motozaki K, Matsuyama S. Human brain carboxypeptidase B, which cleaves beta-amyloid peptides in vitro, is expressed in the endoplasmic reticulum of neurons. Eur J Neurosci May 2001;13(9):1653-7

Intracellular localization of novel human brain carboxypeptidase B (HBCPB) was investigated in human hippocampus, using immunohistochemistry by confocal laser microscopy and biochemical purification of the homogenate by density gradient ultracentrifugation. The former revealed that the majority of HBCPB was expressed in the endoplasmic reticulum, in which the HBCPB-specific C14-module immunoreactivity was colocalized with GRP78 immunoreactivity, a stress 70 heat shock protein specifically expressed in the endoplasmic reticulum. The latter showed that anti-C14-module immunoreactivity and prepro-HBCPB immunoreactivity were both enriched in the microsome fraction, especially in that of the endoplasmic reticulum-density fraction of normal human hippocampal homogenates from various sources. However, HBCPB prepared from human hippocampus showed exopeptidase activity for synthetic beta-amyloid 1-42 peptide, in which Abeta X-42 C-terminus immunoreactivity was decreased in a fashion dose-dependent of the amount of the protease added. These findings indicate that HBCPB, which is expressed in the endoplasmic reticulum of a group of neuronal perikarya, may play an important physiological role in degradation of beta-amyloid 1-42, which is specifically generated in the endoplasmic reticulum of human and rodent neurons and is also regarded as the most pathogenic and aggregatable species among all beta-amyloid peptides.

General_screening_panel_v1.4 Summary: Ag4136 Significant expression of the NOV17 gene, a carboxypeptidase B homolog, is restricted to pancreas and bladder (CTs=20-22). Thus, expression of this gene could be used to differentiate between these samples and other samples on this panel and as a marker of these tissues.

Panel 4.1D Summary: Ag4136 Expression of the NOV17 gene, a carboxypeptidase B homolog is limited to a few samples, with highest expression in the kidney (CT=29.6). Therefore, antibody or small molecule therapies designed with the protein encoded for by this gene could modulate kidney function and be important in the treatment of inflammatory or autoimmune diseases that affect the kidney, including lupus and glomerulonephritis. The NOV17 gene is also expressed at moderate levels in KU-812 basophil cells treated with PMA/ionomycin and at lower levels in untreated basophils. These cells are a reasonable model for the inflammatory cells that take part in various inflammatory lung and bowel diseases, such as asthma, Crohn's disease, and ulcerative colitis. Therefore, therapeutic modulation of the expression or function of this gene may also be effective in the treatment of these diseases.

K. NOV5—CG57081-01: Serine/Threonine Kinase

Expression of the NOV5 gene was assessed using the primer-probe set Ag3072, described in Table KA.

TABLE KA

Probe Name Ag3072

| | | | | |
|---|---|---|---|---|
| Primers | Sequences | Length | Start Position | SEQ ID NO: |

| | | | | |
|---|---|---|---|---|
| Forward | 5′-tggtggtagacctgcttctg-3′ | 20 | 536 | 503 |
| Probe | TET-5′-gacctacgttaccacctgcagcagaa-3′-TAMRA | 26 | 562 | 504 |
| Reverse | 5′-ctcactgtgtcctcggagaa-3′ | 20 | 595 | 505 |

CNS_neurodegeneration_v1.0 Summary: Ag3072 Expression of the NOV5 gene is low/undetectable in all samples on this panel (CTs>35). (Data not shown.)

Panel 1.3D Summary: Ag3072 Expression of the NOV5 gene is low/undetectable in all samples on this panel (CTs>35). (Data not shown.)

Panel 2.2 Summary: Ag3072 Expression of the NOV5 gene is low/undetectable in all samples on this panel (CTs>35). (Data not shown.)

Panel 4D Summary: Ag3072 Expression of the NOV5 gene is low/undetectable in all samples on this panel (CTs>35). (Data not shown.)

L. NOV37—CG57335-01: Protocadherin Beta 3

Expression of the NOV37 gene was assessed using the primer-probe set Ag3192, described in Table LA. Results of the RTQ-PCR runs are shown in Tables LB and LC.

TABLE LA

Probe Name Ag3192

| | | | | |
|---|---|---|---|---|
| Primers | Sequences | Length | Start Position | SEQ ID NO: |

| | | | | |
|---|---|---|---|---|
| Forward | 5′-ctggtacggattgaagttgtg-3′ | 21 | 1107 | 506 |
| Probe | TET-5′-catcaatgacaacgtcccagagtt-3′-TAMRA | 24 | 1130 | 507 |
| Reverse | 5′-gttccaatgtctaaatccctg-3′ | 21 | 1226 | 508 |

TABLE LB
Panel 1.3D

| | Rel. Exp. (%) | Rel. Exp. (%) | | Rel. Exp. (%) | Rel. Exp. (%) |
|---|---|---|---|---|---|
| | Ag3192, | Ag3192, | | Ag3192, | Ag3192, |
| Tissue Name | Run 165673603 | Run 167994640 | Tissue Name | Run 165673603 | Run 167994640 |
| Liver | 59.0 | 19.6 | Kidney (fetal) | 8.7 | 6.2 |
| adenocarcinoma | | | | | |
| Pancreas | 2.9 | 0.0 | Renal ca. 786-0 | 11.7 | 1.5 |
| Pancreatic ca. | 0.0 | 0.0 | Renal ca. A498 | 24.8 | 4.7 |
| CAPAN2 | | | | | |
| Adrenal gland | 2.2 | 0.0 | Renal ca. RXF | 0.0 | 0.0 |
| | | | 393 | | |
| Thyroid | 3.2 | 0.3 | Renal ca. | 0.0 | 0.0 |
| | | | ACHN | | |
| Salivary gland | 2.0 | 0.5 | Renal ca. UO- | 14.0 | 1.9 |
| | | | 31 | | |
| Pituitary gland | 12.6 | 2.0 | Renal ca. TK- | 10.1 | 9.2 |
| | | | 10 | | |
| Brain (fetal) | 33.9 | 8.1 | Liver | 3.9 | 0.0 |
| Brain (whole) | 17.3 | 3.7 | Liver (fetal) | 0.0 | 0.0 |
| Brain (amygdala) | 23.8 | 2.6 | Liver ca. | 24.3 | 4.4 |
| | | | (hepatoblast) | | |
| | | | HepG2 | | |
| Brain (cerebellum) | 40.6 | 3.2 | Lung | 10.3 | 0.8 |
| Brain | 15.5 | 2.9 | Lung (fetal) | 9.2 | 3.1 |
| (hippocampus) | | | | | |
| Brain (substantia | 3.1 | 1.7 | Lung ca. (small | 0.0 | 0.0 |
| nigra) | | | cell) LX-1 | | |
| Brain (thalamus) | 12.7 | 1.8 | Lung ca. (small | 8.8 | 2.0 |
| | | | cell) NCI-H69 | | |
| Cerebral Cortex | 26.4 | 5.1 | Lung ca. (s. cell | 100.0 | 100.0 |
| | | | var.) SHP-77 | | |
| Spinal cord | 12.9 | 1.7 | Lung ca. (large | 52.9 | 0.0 |
| | | | cell) NCI-H460 | | |
| glio/astro U87-MG | 4.1 | 1.1 | Lung ca. (non- | 4.2 | 6.6 |
| | | | sm. cell) A549 | | |
| glio/astro U-118- | 40.9 | 4.0 | Lung ca. (non- | 57.4 | 7.7 |
| MG | | | s. cell) NCI-H23 | | |
| astrocytoma | 31.6 | 6.0 | Lung ca. (non- | 0.0 | 0.0 |
| SW1783 | | | s. cell) HOP-62 | | |
| neuro*; met SK-N- | 0.0 | 0.0 | Lung ca. (non- | 31.2 | 15.5 |
| AS | | | s. cl) NCI-H522 | | |
| astrocytoma SF-539 | 54.3 | 14.8 | Lung ca. | 3.3 | 0.3 |
| | | | (squam.) SW | | |
| | | | 900 | | |
| astrocytoma SNB- | 21.2 | 2.4 | Lung ca. | 3.4 | 2.4 |
| 75 | | | (squam.) NCI- | | |
| | | | H596 | | |
| glioma SNB-19 | 98.6 | 17.8 | Mammary | 12.8 | 2.2 |
| | | | gland | | |
| glioma U251 | 55.9 | 6.0 | Breast ca.* | 27.0 | 7.6 |
| | | | (pl. ef) MCF-7 | | |
| glioma SF-295 | 6.3 | 4.4 | Breast ca.* | 0.0 | 0.0 |
| | | | (pl. ef) MDA- | | |
| | | | MB-231 | | |
| Heart (fetal) | 0.0 | 1.0 | Breast ca.* | 24.8 | 32.8 |
| | | | (pl. ef.) T47D | | |
| Heart | 6.0 | 1.1 | Breast ca. BT- | 75.3 | 3.4 |
| | | | 549 | | |
| Skeletal muscle | 10.6 | 1.2 | Breast ca. | 27.0 | 8.1 |
| (fetal) | | | MDA-N | | |
| Skeletal muscle | 2.8 | 0.3 | Ovary | 10.5 | 2.2 |
| Bone marrow | 0.0 | 0.0 | Ovarian ca. | 15.2 | 1.4 |
| | | | OVCAR-3 | | |
| Thymus | 1.2 | 0.4 | Ovarian ca. | 18.8 | 3.8 |
| | | | OVCAR-4 | | |
| Spleen | 1.5 | 0.3 | Ovarian ca. | 0.0 | 0.0 |
| | | | OVCAR-5 | | |
| Lymph node | 9.3 | 0.0 | Ovarian ca. | 0.0 | 0.7 |
| | | | OVCAR-8 | | |
| Colorectal | 1.8 | 0.4 | Ovarian ca. | 8.5 | 3.5 |
| | | | IGROV-1 | | |
| Stomach | 5.4 | 0.0 | Ovarian ca.* | 56.6 | 71.7 |
| | | | (ascites) SK- | | |
| | | | OV-3 | | |
| Small intestine | 13.2 | 0.9 | Uterus | 31.9 | 1.0 |
| Colon ca. SW480 | 0.0 | 0.0 | Placenta | 0.0 | 0.0 |
| Colon ca.* | 0.0 | 1.5 | Prostate | 5.4 | 0.4 |
| SW620 (SW480 | | | | | |
| met) | | | | | |
| Colon ca. HT29 | 0.0 | 0.0 | Prostate ca.* | 22.4 | 12.6 |
| | | | (bone met) | | |
| | | | PC-3 | | |
| Colon ca. HCT-116 | 2.0 | 0.0 | Testis | 40.9 | 3.8 |
| Colon ca. CaCo-2 | 0.0 | 0.0 | Melanoma | 2.4 | 1.3 |
| | | | Hs688(A).T | | |
| Colon ca. | 10.6 | 0.5 | Melanoma* | 1.9 | 0.8 |
| tissue (ODO3866) | | | (met) | | |
| | | | Hs688(B).T | | |
| Colon ca. HCC- | 1.5 | 0.5 | Melanoma | 30.8 | 11.8 |
| 2998 | | | UACC-62 | | |
| Gastric ca.* (liver | 81.8 | 8.2 | Melanoma M14 | 0.0 | 0.0 |
| met) NCI-N87 | | | | | |
| Bladder | 4.8 | 1.0 | Melanoma | 10.0 | 14.3 |
| | | | LOX IMVI | | |
| Trachea | 4.5 | 0.6 | Melanoma* | 0.0 | 0.9 |
| | | | (met) SK-MEL- | | |
| | | | 5 | | |
| Kidney | 2.2 | 0.8 | Adipose | 4.6 | 1.6 |

TABLE LC
Panel 4D

| | Rel. Exp. (%) | | Rel. Exp. (%) |
|---|---|---|---|
| | Ag3192, | | Ag3192, |
| Tissue Name | Run 164389283 | Tissue Name | Run 164389283 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.8 |
| Secondary Tr1 rest | 0.0 | Lung Microvascular EC none | 0.8 |
| Primary Th1 act | 0.0 | Lung Microvascular EC | 1.4 |
| | | TNFalpha + IL-1beta | |
| Primary Th2 act | 0.0 | Microvascular Dermal EC | 1.6 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 1.6 |
| | | TNFalpha + IL1beta | |
| Primary Th2 rest | 0.0 | Small airway epithelium | 0.0 |
| | | none | |
| Primary Tr1 rest | 0.0 | Small airway epithelium | 12.8 |
| | | TNFalpha + IL-1beta | |
| CD45RA CD4 | 0.7 | Coronery artery SMC rest | 2.5 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 47.0 |
| Secondary CD8 | 0.0 | Astrocytes TNFalpha + IL- | 39.0 |
| lymphocyte rest | | 1beta | |
| Secondary CD8 | 0.0 | KU-812 (Basophil) rest | 12.9 |
| lymphocyte act | | | |
| CD4 lymphocyte none | 0.0 | KU-812 (Basophil) | 25.3 |
| | | PMA/ionomycin | |
| 2ry Th1/Th2/Tr1_anti- | 0.0 | CCD1106 (Keratinocytes) | 0.5 |
| CD95 CH11 | | none | |
| LAK cells rest | 0.0 | CCD1106 (Keratinocytes) | 1.3 |
| | | TNFalpha + IL-1beta | |
| LAK cells IL-2 | 0.5 | Liver cirrhosis | 1.0 |
| LAK cells IL-2 + IL-12 | 0.0 | Lupus kidney | 1.5 |
| LAK cells IL-2 + IFN | 0.0 | NCI-H292 none | 80.1 |
| gamma | | | |
| LAK cells IL-2 + IL-18 | 0.0 | NCI-H292 IL-4 | 70.2 |
| LAK cells | 0.0 | NCI-H292 IL-9 | 100.0 |
| PMA/ionomycin | | | |
| NK Cells IL-2 rest | 0.6 | NCI-H292 IL-13 | 47.0 |
| Two Way MLR 3 day | 0.0 | NCI-H292 IFN gamma | 40.1 |
| Two Way MLR 5 day | 0.0 | HPAEC none | 2.1 |
| Two Way MLR 7 day | 0.0 | HPAEC TNF alpha + IL-1 | 2.7 |
| | | beta | |
| PBMC rest | 0.5 | Lung fibroblast none | 10.2 |
| PBMC PWM | 0.0 | Lung fibroblast TNF alpha + | 2.8 |
| | | IL-1 beta | |
| PBMC PHA-L | 0.0 | Lung fibroblast IL-4 | 13.8 |
| Ramos (B cell) none | 0.0 | Lung fibroblast IL-9 | 7.0 |
| Ramos (B cell) | 0.0 | Lung fibroblast IL-13 | 8.4 |
| ionomycin | | | |
| B lymphocytes PWM | 0.0 | Lung fibroblast IFN gamma | 15.2 |
| B lymphocytes CD40L | 0.0 | Dermal fibroblast CCD1070 | 3.5 |
| and IL-4 | | rest | |
| EOL-1 dbcAMP | 0.0 | Dermal fibroblast CCD1070 | 4.3 |
| | | TNF alpha | |
| EOL-1 dbcAMP | 0.0 | Dermal fibroblast CCD1070 | 3.0 |
| PMA/ionomycin | | IL-1 beta | |
| Dendritic cells none | 0.0 | Dermal fibroblast IFN gamma | 1.5 |
| Dendritic cells LPS | 0.0 | Dermal fibroblast IL-4 | 2.3 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 4.5 |
| Macrophages rest | 0.0 | Lung | 19.6 |
| Macrophages LPS | 0.0 | Thymus | 5.3 |
| HUVEC none | 0.7 | Kidney | 2.2 |
| HUVEC starved | 1.4 | | |

Panel 1.3D Summary: Ag3192 Two experiments with the same probe and primer set produce results that are in reasonable agreement, with highest expression in a lung cancer cell line (CTs=31-33).

Significant levels of expression are also seen in cell lines derived from liver, ovarian, breast, gastric, and brain cancers. Thus, expression of the NOV37 gene could be used to differentiate between these samples and other samples on this panel and as a marker to detect the presence of these cancers. Furthermore, therapeutic modulation of the expression or function of the NOV37 gene may be effective in the treatment of liver, ovarian, breast, gastric, and brain cancers.

In addition, the NOV37 gene, a protocadherin homolog, is detected at low levels in the CNS; levels are highest in the cerebellum. The cadherins have been shown to be critical for CNS development, specifically for the guidance of axons, dendrites and/or growth cones in general. Therapeutic modulation of the levels of this protein, or possible signaling via this protein may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease). Since protocadherins play an important role in synaptogenesis this gene product may also be involved in depression, schizophrenia, which also involve synaptogeneisis. Because this cadherin shows highest expression in the cerebellum, making it an excellent candidate for the spinocerebellar ataxias as well.

### REFERENCES

Hilschmann N, Barnikol H U, Barnikol-Watanabe S, Gotz H, Kratzin H, Thinnes F P. The immunoglobulin-like genetic predetermination of the brain: the protocadherins, blueprint of the neuronal network. Naturwissenschaften January 2001;88(1):2-12

The morphogenesis of the brain is governed by synaptogenesis. Synaptogenesis in turn is determined by cell adhesion molecules, which bridge the synaptic cleft and, by homophilic contact, decide which neurons are connected and which are not. Because of their enormous diversification in specificities, protocadherins (pcdh alpha, pcdh beta, pcdh gamma), a new class of cadherins, play a decisive role. Surprisingly, the genetic control of the protocadherins is very similar to that of the immunoglobulins. There are three sets of variable (V) genes followed by a corresponding constant (C) gene. Applying the rules of the immunoglobulin genes to the protocadherin genes leads, despite of this similarity, to quite different results in the central nervous system. The lymphocyte expresses one single receptor molecule specifically directed against an outside stimulus. In contrast, there are three specific recognition sites in each neuron, each expressing a different protocadherin. In this way, 4,950 different neurons arising from one stem cell form a neuronal network, in which homophilic contacts can be formed in 52 layers, permitting an enormous number of different connections and restraints between neurons. This network is one module of the central computer of the brain. Since the V-genes are generated during evolution and V-gene translocation during embryogenesis, outside stimuli have no influence on this network. The network is an inborn property of the protocadherin genes. Every circuit produced, as well as learning and memory, has to be based on this genetically predetermined network. This network is so universal that it can cope with everything, even the unexpected. In this respect the neuronal network resembles the recognition sites of the immunoglobulins.

Panel 2.2 Summary: Ag3192 Expression of the NOV37 gene is low/undetectable in all samples on this panel (CTs>35). (Data not shown.)

Panel 4D Summary: Ag3192 The NOV37 transcript is expressed in NCI-H292 cells. Treatment of these cells does not seem to significantly alter expression of this transcript in this muco-epidermoid cell line. Thus, the protein could be used to identify certain lung tumors similar to NCI-H292, consistent with panel 1.3. The encoded protein may also contribute to the normal function of the goblet cells within the lung. Therefore, designing therapeutics to this protein may be important for the treament of emphysema and asthma as well as other lung diseases in which goblet cells or the mucus they produce have pathological consequences.

Panel CNS_{—}1 Summary: Ag3192 Expression of the NOV37 gene is low/undetectable in all samples on this panel (CTs>35). (Data not shown.)

M. M28130: Sequence from Methods of Use for Interleukin-8 (IL-8) and Anti-IL-8 Antibodies Patent

Expression of gene IL-8 (GenBank Accesion No.M28130) was assessed using the primer-probe set Ag1016, described in Table MA. Results of the RTQ-PCR runs are shown in Tables MB, MC and MD.

TABLE MA

Probe Name Ag1016

| | | | | |
|---|---|---|---|---|
| Primers | Sequences | Length | Start Position | SEQ ID NO: |

| | | | | |
|---|---|---|---|---|
| Forward | 5′-attgcacgggagaatatacaaa-3′ | 22 | 673 | 509 |
| Probe | TET-5′-ccaagggccaagagaatatccgaact-3′-TAMRA | 26 | 708 | 510 |
| Reverse | 5′-tcacattctagcaaacccattc-3′ | 22 | 749 | 511 |

TABLE MB
AI_comprehensive panel_v1.0

| | Rel. Exp. (%) | Rel. Exp. (%) | | Rel. Exp. (%) | Rel. Exp. (%) |
|---|---|---|---|---|---|
| | Ag1016, Run | Ag1016, Run | | Ag1016, Run | Ag1016, Run |
| Tissue Name | 211059879 | 212309939 | Tissue Name | 211059879 | 212309939 |
| 110967 COPD-F | 0.4 | 0.2 | 112427 Match | 0.1 | 0.1 |
| | | | Control | | |
| | | | Psoriasis-F | | |
| 110980 COPD-F | 0.1 | 0.1 | 112418 | 0.5 | 0.6 |
| | | | Psoriasis-M | | |
| 110968 COPD-M | 0.1 | 0.3 | 112723 Match | 0.0 | 0.0 |
| | | | Control | | |
| | | | Psoriasis-M | | |
| 110977 COPD- | 0.4 | 0.4 | 112419 | 1.6 | 1.9 |
| M | | | Psoriasis-M | | |
| 110989 | 0.0 | 0.1 | 112424 Match | 0.1 | 0.1 |
| Emphysema-F | | | Control | | |
| | | | Psoriasis-M | | |
| 110992 | 0.0 | 0.1 | 112420 | 0.5 | 0.6 |
| Emphysema-F | | | Psoriasis-M | | |
| 110993 | 0.0 | 0.0 | 112425 Match | 0.1 | 0.1 |
| Emphysema-F | | | Control | | |
| | | | Psoriasis-M | | |
| 110994 | 0.0 | 0.0 | 104689 (MF) | 0.2 | 0.1 |
| Emphysema-F | | | OA Bone- | | |
| | | | Backus | | |
| 110995 | 0.0 | 0.1 | 104690 (MF) | 0.4 | 0.2 |
| Emphysema-F | | | Adj “Normal” | | |
| | | | Bone-Backus | | |
| 110996 | 0.0 | 0.1 | 104691 (MF) | 0.1 | 0.0 |
| Emphysema-F | | | OA Synovium- | | |
| | | | Backus | | |
| 110997 Asthma- | 1.0 | 1.1 | 104692 (BA) | 0.1 | 0.1 |
| M | | | OA Cartilage- | | |
| | | | Backus | | |
| 111001 Asthma- | 0.1 | 0.0 | 104694 (BA) | 0.3 | 0.2 |
| F | | | OA Bone- | | |
| | | | Backus | | |
| 111002 Asthma- | 0.1 | 0.0 | 104695 (BA) | 0.0 | 0.1 |
| F | | | Adj “Normal” | | |
| | | | Bone-Backus | | |
| 111003 Atopic | 0.1 | 0.0 | 104696 (BA) | 0.8 | 1.0 |
| Asthma-F | | | OA Synovium- | | |
| | | | Backus | | |
| 111004 Atopic | 0.2 | 0.2 | 104700 (SS) OA | 1.3 | 2.6 |
| Asthma-F | | | Bone-Backus | | |
| 111005 Atopic | 0.1 | 0.1 | 104701 (SS) Adj | 0.3 | 0.3 |
| Asthma-F | | | “Normal” Bone- | | |
| | | | Backus | | |
| 111006 Atopic | 0.0 | 0.0 | 104702 (SS) OA | 0.7 | 0.6 |
| Asthma-F | | | Synovium- | | |
| | | | Backus | | |
| 111417 Allergy- | 0.0 | 0.2 | 117093 OA | 0.5 | 0.6 |
| M | | | Cartilage Rep7 | | |
| 112347 Allergy- | 0.0 | 0.0 | 112672 OA | 0.2 | 0.2 |
| M | | | Bone5 | | |
| 112349 Normal | 0.0 | 0.0 | 112673 OA | 0.1 | 0.1 |
| Lung-F | | | Synovium5 | | |
| 112357 Normal | 0.8 | 0.4 | 112674 OA | 0.2 | 0.1 |
| Lung-F | | | Synovial Fluid | | |
| | | | cells5 | | |
| 112354 Normal | 0.4 | 0.7 | 117100 OA | 0.0 | 0.0 |
| Lung-M | | | Cartilage Rep14 | | |
| 112374 Crohns- | 0.1 | 0.1 | 112756 OA | 2.5 | 2.2 |
| F | | | Bone9 | | |
| 112389 Match | 0.1 | 0.0 | 112757 OA | 0.0 | 0.1 |
| Control Crohns- | | | Synovium9 | | |
| F | | | | | |
| 112375 Crohns- | 0.0 | 0.0 | 112758 OA | 0.1 | 0.1 |
| F | | | Synovial Fluid | | |
| | | | Cells9 | | |
| 112732 Match | 1.1 | 0.9 | 117125 RA | 0.1 | 0.0 |
| Control Crohns- | | | Cartilage Rep2 | | |
| F | | | | | |
| 112725 Crohns- | 0.0 | 0.1 | 113492 Bone2 | 6.0 | 7.2 |
| M | | | RA | | |
| 112387 Match | 0.0 | 0.0 | 113493 | 1.7 | 1.5 |
| Control Crohns- | | | Synovium2 RA | | |
| M | | | | | |
| 112378 Crohns- | 0.0 | 0.0 | 113494 Syn | 3.4 | 3.6 |
| M | | | Fluid Cells RA | | |
| 112390 Match | 0.0 | 0.0 | 113499 | 5.6 | 4.9 |
| Control Crohns- | | | Cartilage4 RA | | |
| M | | | | | |
| 112726 Crohns- | 0.2 | 0.2 | 113500 Bone4 | 6.1 | 4.4 |
| M | | | RA | | |
| 112731 Match | 0.4 | 0.4 | 113501 | 3.3 | 4.3 |
| Control Crohns- | | | Synovium4 RA | | |
| M | | | | | |
| 112380 Ulcer | 0.0 | 0.2 | 113502 Syn | 3.1 | 2.5 |
| Col-F | | | Fluid Cells4 RA | | |
| 112734 Match | 100.0 | 100.0 | 113495 | 3.0 | 3.0 |
| Control Ulcer | | | Cartilage3 RA | | |
| Col-F | | | | | |
| 112384 Ulcer | 0.3 | 0.2 | 113496 Bone3 | 3.2 | 3.7 |
| Col-F | | | RA | | |
| 112737 Match | 0.1 | 0.5 | 113497 | 1.7 | 2.1 |
| Control Ulcer | | | Synovium3 RAN | | |
| Col-F | | | | | |
| 112386 Ulcer | 0.0 | 0.1 | 113498 Syn | 4.8 | 4.1 |
| Col-F | | | Fluid Cells3 RA | | |
| 112738 Match | 2.3 | 2.6 | 117106 Normal | 0.1 | 0.0 |
| Control Ulcer | | | Cartilage Rep20 | | |
| Col-F | | | | | |
| 112381 Ulcer | 0.0 | 0.1 | 113663 Bone3 | 0.0 | 0.0 |
| Col-M | | | Normal | | |
| 112735 Match | 0.1 | 0.5 | 113664 | 0.0 | 0.0 |
| Control Ulcer | | | Synovium3 | | |
| Col-M | | | Normal | | |
| 112382 Ulcer | 0.4 | 0.3 | 113665 Syn | 0.0 | 0.0 |
| Col-M | | | Fluid Cells3 | | |
| | | | Normal | | |
| 112394 Match | 0.0 | 0.0 | 117107 Normal | 0.0 | 0.0 |
| Control Ulcer | | | Cartilage Rep22 | | |
| Col-M | | | | | |
| 112383 Ulcer | 0.8 | 0.7 | 113667 Bone4 | 0.1 | 0.1 |
| Col-M | | | Normal | | |
| 112736 Match | 0.1 | 0.0 | 113668 | 0.1 | 0.1 |
| Control Ulcer | | | Synovium4 | | |
| Col-M | | | Normal | | |
| 112423 | 7.1 | 7.6 | 113669 Syn | 0.2 | 0.2 |
| Psoriasis-F | | | Fluid Cells4 | | |
| | | | Normal | | |

TABLE MC
General_screening_panel_v1.4

| | Rel. Exp. (%) | Rel. Exp. (%) | | Rel. Exp. (%) | Rel. Exp. (%) |
|---|---|---|---|---|---|
| | Ag1016, Run | Ag1016, Run | | Ag1016, Run | Ag1016, Run |
| Tissue Name | 208030041 | 212141063 | Tissue Name | 208030041 | 212141063 |
| Adipose | 2.5 | 3.4 | Renal ca. TK-10 | 8.9 | 11.7 |
| Melanoma* | 0.1 | 0.0 | Bladder | 1.6 | 4.7 |
| Hs688(A).T | | | | | |
| Melanoma* | 0.1 | 0.1 | Gastric ca. (liver | 0.2 | 0.5 |
| Hs688(B).T | | | met.) NCI-N87 | | |
| Melanoma* | 0.0 | 0.0 | Gastric ca. KATO | 3.1 | 10.7 |
| M14 | | | III | | |
| Melanoma* | 46.0 | 54.0 | Colon ca. SW-948 | 2.3 | 8.8 |
| LOXIMVI | | | | | |
| Melanoma* | 0.3 | 0.7 | Colon ca. SW480 | 0.0 | 0.0 |
| SK-MEL-5 | | | | | |
| Squamous cell | 6.1 | 12.6 | Colon ca.* | 0.8 | 0.3 |
| carcinoma | | | (SW480 met) | | |
| SCC-4 | | | SW620 | | |
| Testis Pool | 0.0 | 0.1 | Colon ca. HT29 | 0.1 | 0.1 |
| Prostate ca.* | 2.1 | 2.5 | Colon ca. HCT- | 0.0 | 0.1 |
| (bone met) PC- | | | 116 | | |
| 3 | | | | | |
| Prostate Pool | 0.2 | 0.2 | Colon ca. CaCo-2 | 0.0 | 0.0 |
| Placenta | 0.0 | 0.0 | Colon cancer | 100.0 | 100.0 |
| | | | tissue | | |
| Uterus Pool | 0.0 | 0.0 | Colon ca. SW1116 | 0.0 | 0.0 |
| Ovarian ca. | 0.1 | 0.4 | Colon ca. Colo- | 0.0 | 0.0 |
| OVCAR-3 | | | 205 | | |
| Ovarian ca. | 1.3 | 5.7 | Colon ca. SW-48 | 0.0 | 0.0 |
| SK-OV-3 | | | | | |
| Ovarian ca. | 1.0 | 2.8 | Colon Pool | 0.0 | 0.0 |
| OVCAR-4 | | | | | |
| Ovarian ca. | 0.1 | 0.5 | Small Intestine | 0.0 | 0.1 |
| OVCAR-5 | | | Pool | | |
| Ovarian ca. | 0.0 | 0.1 | Stomach Pool | 0.2 | 1.3 |
| IGROV-1 | | | | | |
| Ovarian ca. | 0.1 | 0.2 | Bone Marrow | 0.2 | 0.3 |
| OVCAR-8 | | | Pool | | |
| Ovary | 0.0 | 0.0 | Fetal Heart | 0.0 | 0.0 |
| Breast ca. | 0.0 | 0.0 | Heart Pool | 0.0 | 0.0 |
| MCF-7 | | | | | |
| Breast ca. | 0.6 | 1.0 | Lymph Node Pool | 0.0 | 0.0 |
| MDA-MB-231 | | | | | |
| Breast ca. BT | 3.2 | 0.0 | Fetal Skeletal | 0.0 | 0.1 |
| 549 | | | Muscle | | |
| Breast ca. | 0.2 | 0.8 | Skeletal Muscle | 0.1 | 0.1 |
| T47D | | | Pool | | |
| Breast ca. | 0.1 | 0.2 | Spleen Pool | 0.1 | 0.5 |
| MDA-N | | | | | |
| Breast Pool | 0.1 | 0.1 | Thymus Pool | 0.3 | 1.1 |
| Trachea | 4.5 | 7.8 | CNS cancer | 96.6 | 94.0 |
| | | | (glio/astro) U87- | | |
| | | | MG | | |
| Lung | 0.1 | 0.2 | CNS cancer | 26.6 | 33.4 |
| | | | (glio/astro) U-118- | | |
| | | | MG | | |
| Fetal Lung | 4.6 | 5.7 | CNS cancer | 0.0 | 0.1 |
| | | | (neuro; met) SK- | | |
| | | | N-AS | | |
| Lung ca. NCI- | 0.0 | 0.0 | CNS cancer | 0.0 | 0.0 |
| N417 | | | (astro) SF-539 | | |
| Lung ca. LX-1 | 2.2 | 1.3 | CNS cancer | 0.5 | 1.3 |
| | | | (astro) SNB-75 | | |
| Lung ca. NCI- | 0.0 | 0.0 | CNS cancer (glio) | 0.0 | 0.2 |
| H146 | | | SNB-19 | | |
| Lung ca. SUP- | 1.7 | 2.1 | CNS cancer (glio) | 6.1 | 10.8 |
| 77 | | | SF-295 | | |
| Lung ca. A549 | 0.4 | 0.3 | Brain (Amygdala) | 0.0 | 0.1 |
| | | | Pool | | |
| Lung ca. NCI- | 0.0 | 0.0 | Brain (cerebellum) | 0.0 | 0.0 |
| H526 | | | | | |
| Lung ca. NCI- | 1.2 | 2.6 | Brain (fetal) | 0.1 | 0.4 |
| H23 | | | | | |
| Lung ca. NCI- | 22.4 | 85.9 | Brain | 1.4 | 1.5 |
| H460 | | | (Hippocampus) | | |
| | | | Pool | | |
| Lung ca. HOP- | 2.1 | 7.3 | Cerebral Cortex | 0.1 | 0.2 |
| 62 | | | Pool | | |
| Lung ca. NCI- | 0.0 | 0.0 | Brain (Substantia | 0.1 | 0.2 |
| H522 | | | nigra) Pool | | |
| Liver | 0.0 | 0.0 | Brain (Thalamus) | 0.0 | 0.2 |
| | | | Pool | | |
| Fetal Liver | 0.0 | 0.0 | Brain (whole) | 0.0 | 0.1 |
| Liver ca. | 0.0 | 0.0 | Spinal Cord Pool | 0.3 | 1.1 |
| HepG2 | | | | | |
| Kidney Pool | 0.1 | 0.3 | Adrenal Gland | 0.1 | 0.4 |
| Fetal Kidney | 0.0 | 0.1 | Pituitary gland | 0.0 | 0.0 |
| | | | Pool | | |
| Renal ca. 786-0 | 0.3 | 1.1 | Salivary Gland | 0.2 | 0.1 |
| Renal ca. A498 | 0.0 | 0.0 | Thyroid (female) | 0.1 | 0.3 |
| Renal ca. | 1.9 | 6.3 | Pancreatic ca. | 0.3 | 0.7 |
| ACHN | | | CAPAN2 | | |
| Renal ca. UO- | 24.5 | 63.7 | Pancreas Pool | 0.4 | 0.7 |
| 31 | | | | | |

TABLE MD
Panel 4.1D

| | Rel. Exp. (%) | Rel. Exp. (%) | | Rel. Exp. (%) | Rel. Exp. (%) |
|---|---|---|---|---|---|
| | Ag1016, Run | Ag1016, Run | | Ag1016, Run | Ag1016, Run |
| Tissue Name | 170806468 | 246331464 | Tissue Name | 170806468 | 246331464 |
| Secondary Th1 act | 1.2 | 1.5 | HUVEC IL-1beta | 6.8 | 8.2 |
| Secondary Th2 act | 0.2 | 0.3 | HUVEC IFN | 0.2 | 0.4 |
| | | | gamma | | |
| Secondary Tr1 act | 0.4 | 0.5 | HUVEC TNF alpha + | 4.8 | 4.6 |
| | | | IFN gamma | | |
| Secondary Th1 rest | 0.0 | 0.0 | HUVEC TNF alpha + | 2.5 | 2.6 |
| | | | IL4 | | |
| Secondary Th2 rest | 0.0 | 0.0 | HUVEC IL-11 | 0.0 | 0.0 |
| Secondary Tr1 rest | 0.0 | 0.0 | Lung Microvascular | 0.1 | 0.1 |
| | | | EC none | | |
| Primary Th1 act | 0.0 | 0.0 | Lung Microvascular | 13.3 | 11.2 |
| | | | EC TNFalpha + IL- | | |
| | | | 1beta | | |
| Primary Th2 act | 0.0 | 0.0 | Microvascular | 0.2 | 0.2 |
| | | | Dermal EC none | | |
| Primary Tr1 act | 0.1 | 0.1 | Microsvasular | 6.8 | 7.7 |
| | | | Dermal EC | | |
| | | | TNFalpha + IL- | | |
| | | | 1beta | | |
| Primary Th1 rest | 0.0 | 0.0 | Bronchial | 2.1 | 1.0 |
| | | | epithelium | | |
| | | | TNFalpha + | | |
| | | | IL1beta | | |
| Primary Th2 rest | 0.0 | 0.0 | Small airway | 0.2 | 0.2 |
| | | | epithelium none | | |
| Primary Tr1 rest | 0.0 | 0.0 | Small airway | 3.5 | 4.3 |
| | | | epithelium | | |
| | | | TNFalpha + IL- | | |
| | | | 1beta | | |
| CD45RA CD4 | 4.5 | 5.1 | Coronery artery | 11.5 | 12.7 |
| lymphocyte act | | | SMC rest | | |
| CD45RO CD4 | 0.1 | 0.1 | Coronery artery | 19.1 | 14.0 |
| lymphocyte act | | | SMC TNFalpha + | | |
| | | | IL-1beta | | |
| CD8 lymphocyte act | 0.0 | 0.0 | Astrocytes rest | 0.1 | 0.1 |
| Secondary CD8 | 0.0 | 0.0 | Astrocytes | 2.9 | 3.8 |
| lymphocyte rest | | | TNFalpha + IL- | | |
| | | | 1beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 0.5 | 0.7 |
| none | | | PMA/ionomycin | | |
| 2ry | 0.0 | 0.0 | CCD1106 | 0.0 | 0.2 |
| Th1/Th2/Tr1_anti- | | | (Keratinocytes) | | |
| CD95 CH11 | | | none | | |
| LAK cells rest | 0.2 | 0.2 | CCD1106 | 0.0 | 0.0 |
| | | | (Keratinocytes) | | |
| | | | TNFalpha + IL- | | |
| | | | 1beta | | |
| LAK cells IL-2 | 0.0 | 0.0 | Liver cirrhosis | 0.1 | 0.1 |
| LAK cells IL-2 + IL- | 0.2 | 0.3 | NCI-H292 none | 0.1 | 0.0 |
| 12 | | | | | |
| LAK cells IL- | 0.1 | 0.1 | NCI-H292 IL-4 | 0.0 | 0.0 |
| 2 + IFN gamma | | | | | |
| LAK cells IL-2 + IL- | 0.1 | 0.1 | NCI-H292 IL-9 | 0.2 | 0.1 |
| 18 | | | | | |
| LAK cells | 33.2 | 41.8 | NCI-H292 IL-13 | 0.2 | 0.6 |
| PMA/ionomycin | | | | | |
| NK Cells IL-2 rest | 0.0 | 0.0 | NCI-H292 IFN | 0.1 | 0.0 |
| | | | gamma | | |
| Two Way MLR 3 | 1.6 | 1.8 | HPAEC none | 2.0 | 0.2 |
| day | | | | | |
| Two Way MLR 5 | 1.3 | 1.3 | HPAEC TNF alpha + | 18.2 | 28.5 |
| day | | | IL-1 beta | | |
| Two Way MLR 7 | 0.2 | 0.2 | Lung fibroblast | 0.5 | 0.0 |
| day | | | none | | |
| PBMC rest | 0.3 | 0.2 | Lung fibroblast | 25.0 | 23.8 |
| | | | TNFalpha + IL-1 | | |
| | | | beta | | |
| PBMC PWM | 5.2 | 8.7 | Lung fibroblast IL- | 0.0 | 0.0 |
| | | | 4 | | |
| PBMC PHA-L | 5.8 | 5.1 | Lung fibroblast IL- | 0.1 | 0.1 |
| | | | 9 | | |
| Ramos (B cell) none | 0.0 | 0.0 | Lung fibroblast IL- | 0.1 | 0.1 |
| | | | 13 | | |
| Ramos (B cell) | 0.0 | 0.0 | Lung fibroblast IFN | 0.1 | 0.4 |
| ionomycin | | | gamma | | |
| B lymphocytes | 0.1 | 0.1 | Dermal fibroblast | 0.5 | 1.5 |
| PWM | | | CCD1070 rest | | |
| B lymphocytes | 0.2 | 0.1 | Dermal fibroblast | 17.3 | 19.8 |
| CD40L and IL-4 | | | CCD1070 TNF | | |
| | | | alpha | | |
| EOL-1 dbcAMP | 0.6 | 0.7 | Dermal fibroblast | 32.1 | 37.4 |
| | | | CCD1070 IL-1 beta | | |
| EOL-1 dbcAMP | 7.7 | 8.01 | Dermal fibroblast | 2.6 | 0.4 |
| PMA/ionomycin | | | IFN gamma | | |
| Dendritic cells none | 0.0 | 0.0 | Dermal fibroblast | 0.4 | 1.0 |
| | | | IL-4 | | |
| Dendritic cells LPS | 2.8 | 2.1 | Dermal Fibroblasts | 2.2 | 2.9 |
| | | | rest | | |
| Dendritic cells anti- | 0.0 | 0.0 | Neutrophils | 53.6 | 63.3 |
| CD40 | | | TNFa + LPS | | |
| Monocytes rest | 0.3 | 0.3 | Neutrophils rest | 2.1 | 6.2 |
| Monocytes LPS | 100.0 | 100.0 | Colon | 0.0 | 0.2 |
| Macrophages rest | 0.3 | 0.2 | Lung | 0.3 | 0.5 |
| Macrophages LPS | 33.4 | 22.2 | Thymus | 0.2 | 0.1 |
| HUVEC none | 0.0 | 0.0 | Kidney | 0.1 | 0.1 |
| HUVEC starved | 0.0 | 0.0 | | | |

AI_comprehensive panel_v1.0 Summary: Expression of IL-8 is widespread in this panel confirming the presence of IL-8 in samples related to the autoimmune response.

General_screening_panel_v1.4 Summary: Prominent expression of this gene, an IL-8 homoolg, on this panel is seen in cancer cell lines, including samples derived from brain, lung, colon, renal and melanoma cancers. Because of the published role of IL-8 in mediating angiogenesis, combination therapies with experimental or established anti-angiogenic drugs, monoclonal antibodies and/or protein therapeutics is anticipated to display synergistic efficacy in a clinical setting and be effective in the treatment of these cancers.

Panel 4.1D Summary: The samples in this panel differ slightly from those in Panel 4D, with samples derived from neutrophils present only on Panel 4.1 D. Furthermore IL-8 is upregulated significantly in TNF-alpha/LPS treated neutrophils when compared to expression in resting neutrophils. The expression of IL-8 is also upregulated significantly in the following immune-stimulated cell types relative to their resting counterparts: dermal fibroblasts treated with IL-1 beta or TNF alpha; microvascular endothelial cells treated with IL-1 beta and TNF alpha; lung fibroblasts treated with IL-1 beta and TNF alpha; pulmonary artery endothelial cells treated with IL-1 beta and TNF alpha; astrocytes treated with IL-1 beta and TNF alpha; small airway epithelium treated with IL-1 beta and TNF alpha; HUVEC's treated with TNF alpha or IL-1 beta; LPS treated macrophages, monocytes and dendritic cells, activated eosinophils and peripheral blood mononuclear cells; and finally LAK cells stimulated with PMA/ionomycin. The secretion of IL-8 by endothelial cells upon stimulation with inflammatory cytokines TNF alpha and IL-1-Beta indicates that IL-8 may be involved in the arrest of neutrophils and perhaps monocytes or endothelial cells, as well as the subsequent transendothelial migration of these cells. Therefore small molecule antagonists or blocking mAbs to IL-8 may be potential therapeutics in acute inflammatory diseases where neutrophils play an important role, such as ischemia reperfusion in the heart, intestine and brain, as well as in endotoxic shock and ARDS. Neutrophils are also thought to play an important role in one chronic inflammatory disease, emphysema (COPD), which could also be treated with IL-8 antagonists. In chronic inflammatory diseases with an immune component these antagonists may prevent the trafficking of monocytes to the area of inflammation. This would eventually lead to a loss of Antigen Presenting Cells at the site of inflammation as monocytes can differentiate into dendritic cells. As a result, the immune response would be down-regulated and the inflammation subside. It is also known that monocytes differentiate into macrophages at the site of inflammation. These cells are a major source of inflammatory cytokines such as TNF alpha and IL1 beta, which contribute to the inflammation. Therefore, blockade of monocyte migration to the site over time will deplete macrophages and result in a decrease in the production of pro-inflammatory cytokines at the site and as a result, the inflammation will decrease. Rheumatoid arthritis, inflammatory bowel disease, asthma, atopic dermatitis, psoriasis, and multiple sclerosis all could be treated with IL8 antagonists to antagonize monocyte trafficking. In summary the data shows that IL-8 is target for antibody-mediated therapy for multiple inflammatory diseases including psoriasis, asthma, allergy, emphysema, stroke, ischemia reperfusion injury, encephalitis, AIDS-related dementia and septic shock.

Based on data provided in panel 1.3 and panel 2D therapy directed against soluble IL-8 is anticipated to have a pronounced impact on the malignant progression of the following human tumors; adenocarcinomas of the colon, squamous cell and adenocarcinomas of the lung, clear cell renal cell carcinomas, hepatocellular carcinomas, transitional cell carcinomas of the bladder, and Cystadenocarcinoma and adenocarcinomas of the stomach, ovarian tumors and thyroid tumors. Panel 1.3 also suggests applicability to the treatment of gliomas and astrocytomas. Therapy could be applied clinically using a monoclonal antibody immuno-specifically recognizing (i.e. binding to, interacting with) IL-8. Such antibody could be conjugated to a prodrug-activating enzyme, a radioisotope, or any number of toxins that have been applied in pre-clinical animal tumor xenograft models. Therapy might also be applied by a tumor homing adenovirus or other viral vector system expressing a “ribozyme” designed to specifically target the IL-8 messenger RNA molecule (transcript) for hydrolytic degradation. Likewise, modified or unmodified antisense oligonucleotides designed to disrupt IL-8 mRNA stability and/or translation, that have been targeted to these tumors by various technologies (liposomes, tumor vascular homing peptides, direct intratumoral injection and/or electroporation) would be anticipated to retard or block disease progression. Because of the published role of IL-8 in mediating angiogenesis, combination therapies with experimental or established anti-angiogenic drugs, monoclonal antibodies and/or protein therapeutics is anticipated to display synergistic efficacy in a clinical setting.

Following physical trauma to the brain and spinal cord, leukocytes are quickly recruited to the damaged area and surrounding tissue. Such cells are thought to be involved in the instigation and perpetuation of local inflammatory responses (macrophage recruitment, infiltration and activation; free radical production) which further exacerbate tissue injury. There is evidence that the same mechanisms also operate in stroke, AIDS dementia, inflammatory peripheral neuropathies and other conditions of CNS encephalities.

IL-8 is also a therapeutic target in meningitis where it is involved in leukocyte recruitment.

With respect to demyelination diseases, antibodies to IL-8 may also have therapeutic use in multiple sclerosis, cerebral lupus and other demyelinating disorders of the CNS. eentry of leukocytes is critical for extracellular proteolysis the development of antibody-producing cells that synthesize antibodies against myelin proteins, as well as the recruitment of macrophages to plaque sites in the cerebral white matter. (Cuzner and Opdenakker: J. Neuroimmunol., 94:1-14, 1999).

N. NOV22—CG57256-01 and CG57256-02: Protein Tyrosine Phosphatase

Expression of the NOV22 genes was assessed using the primer-probe set Ag3272, described in Table NA. Results of the RTQ-PCR runs are shown in Tables NB, NC and ND.

TABLE NA

Probe Name Ag3272

| | | | | |
|---|---|---|---|---|
| | | | | SEQ ID |
| Primers | Sequences | Length | Start Position | NO: |

| | | | | |
|---|---|---|---|---|
| Forward | 5′-tgccctagcatcagttgaag | 20 | 365 | 512 |
| Probe | TET-5′-tggaatgaaacatgaagatgcagtaca-3′-TAMRA | 27 | 386 | 513 |
| Reverse | 5′-tttaaaagctccactccgct-3′ | 20 | 427 | 514 |

TABLE NB
CNS_neurodegeneration_v1.0

| | Rel. Exp. (%) | | Rel. Exp. (%) |
|---|---|---|---|
| | Ag3272, Run | | Ag3272, Run |
| Tissue Name | 210038590 | Tissue Name | 210038590 |
| AD 1 Hippo | 0.0 | Control (Path) 3 | 0.0 |
| | | Temporal Ctx | |
| AD 2 Hippo | 0.0 | Control (Path) 4 | 0.0 |
| | | Temporal Ctx | |
| AD 3 Hippo | 0.0 | AD 1 Occipital Ctx | 0.0 |
| AD 4 Hippo | 0.0 | AD 2 Occipital Ctx | 0.0 |
| | | (Missing) | |
| AD 5 Hippo | 0.0 | AD 3 Occipital Ctx | 0.0 |
| AD 6 Hippo | 0.0 | AD 4 Occipital Ctx | 0.0 |
| Control 2 Hippo | 0.1 | AD 5 Occipital Ctx | 0.0 |
| Control 4 Hippo | 0.1 | AD 6 Occipital Ctx | 0.0 |
| Control (Path) 3 | 0.0 | Control 1 Occipital | 0.0 |
| Hippo | | Ctx | |
| AD 1 Temporal Ctx | 0.1 | Control 2 Occipital | 0.0 |
| | | Ctx | |
| AD 2 Temporal Ctx | 0.1 | Control 3 Occipital | 0.0 |
| | | Ctx | |
| AD 3 Temporal Ctx | 0.0 | Control 4 Occipital | 0.0 |
| | | Ctx | |
| AD 4 Temporal Ctx | 100.0 | Control (Path) 1 | 0.3 |
| | | Occipital Ctx | |
| AD 5 Inf Temporal | 0.1 | Control (Path) 2 | 0.1 |
| Ctx | | Occipital Ctx | |
| AD 5 Sup Temporal | 0.0 | Control (Path) 3 | 0.0 |
| Ctx | | Occipital Ctx | |
| AD 6 Inf Temporal | 0.0 | Control (Path) 4 | 0.0 |
| Ctx | | Occipital Ctx | |
| AD 6 Sup Temporal | 0.0 | Control 1 Parietal Ctx | 0.0 |
| Ctx | | | |
| Control 1 Temporal | 0.0 | Control 2 Parietal Ctx | 0.1 |
| Ctx | | | |
| Control 2 Temporal | 0.0 | Control 3 Parietal Ctx | 0.0 |
| Ctx | | | |
| Control 3 Temporal | 0.0 | Control (Path) 1 | 0.0 |
| Ctx | | Parietal Ctx | |
| Control 3 Temporal | 0.0 | Control (Path) 2 | 0.0 |
| Ctx | | Parietal Ctx | |
| Control (Path) 1 | 0.1 | Control (Path) 3 | 0.0 |
| Temporal Ctx | | Parietal Ctx | |
| Control (Path) 2 | 0.1 | Control (Path) 4 | 0.2 |
| Temporal Ctx | | Parietal Ctx | |

TABLE NC
General_screening_panel_v1.4

| | Rel. Exp. (%) | | Rel. Exp. (%) |
|---|---|---|---|
| | Ag3272, Run | | Ag3272, Run |
| Tissue Name | 215775344 | Tissue Name | 215775344 |
| Adipose | 0.0 | Renal ca. TK-10 | 0.0 |
| Melanoma* | 0.0 | Bladder | 0.0 |
| Hs688(A).T | | | |
| Melanoma* | 0.3 | Gastric ca. (liver met.) | 0.1 |
| Hs688(B).T | | NCI-N87 | |
| Melanoma* M14 | 0.0 | Gastric ca. KATO III | 0.0 |
| Melanoma* | 0.1 | Colon ca. SW-948 | 100.0 |
| LOXIMVI | | | |
| Melanoma* SK-MEL- | 0.0 | Colon ca. SW480 | 0.0 |
| 5 | | | |
| Squamous cell | 0.0 | Colon ca.* (SW480 met) | 0.0 |
| carcinoma SCC-4 | | SW620 | |
| Testis Pool | 0.0 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 0.0 |
| Placenta | 2.6 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 0.0 |
| Ovarian ca. OVCAR-5 | 0.0 | Small Intestine Pool | 0.0 |
| Ovarian ca. IGROV-1 | 0.0 | Stomach Pool | 0.1 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB- | 0.0 | Lymph Node Pool | 0.1 |
| 231 | | | |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.1 |
| Trachea | 0.0 | CNS cancer (glio/astro) | 1.9 |
| | | U87-MG | |
| Lung | 0.0 | CNS cancer (glio/astro) | 0.1 |
| | | U-118-MG | |
| Fetal Lung | 0.0 | CNS cancer (neuro; met) | 0.0 |
| | | SK-N-AS | |
| Lung ca. NCI-N417 | 0.0 | CNS cancer (astro) SF- | 0.1 |
| | | 539 | |
| Lung ca. LX-1 | 0.0 | CNS cancer (astro) SNB- | 0.5 |
| | | 75 | |
| Lung ca. NCI-H146 | 0.0 | CNS cancer (glio) SNB- | 0.0 |
| | | 19 | |
| Lung ca. SHP-77 | 0.1 | CNS cancer (glio) SF- | 3.4 |
| | | 295 | |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.3 | Brain (fetal) | 0.2 |
| Lung ca. NCI-H460 | 2.2 | Brain (Hippocampus) | 0.0 |
| | | Pool | |
| Lung ca. HOP-62 | 0.0 | Cerebral Cortex Pool | 0.0 |
| Lung ca. NCI-H522 | 0.0 | Brain (Substantia nigra) | 0.0 |
| | | Pool | |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.1 |
| Kidney Pool | 0.0 | Adrenal Gland | 0.0 |
| Fetal Kidney | 0.9 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.1 | Pancreatic ca. CAPAN2 | 0.0 |
| Renal ca. UO-31 | 0.0 | Pancreas Pool | 0.0 |

TABLE ND
Panel 4D

| | Rel. Exp. (%) | | Rel. Exp. (%) |
|---|---|---|---|
| | Ag3272, Run | | Ag3272, Run |
| Tissue Name | 165128063 | Tissue Name | 165128063 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 6.8 | Bronchial epithelium | 6.6 |
| | | TNFalpha + IL-1beta | |
| Primary Th2 rest | 0.0 | Small airway epithelium | 0.0 |
| | | none | |
| Primary Tr1 rest | 0.0 | Small airway epithelium | 5.3 |
| | | TNFalpha + IL-1beta | |
| CD45RA CD4 | 0.0 | Coronery artery SMC rest | 0.0 |
| lymphocyte act | | | |
| CD45RO CD4 | 7.8 | Coronery artery SMC | 0.0 |
| lymphocyte act | | TNFalpha + IL-1beta | |
| CD8 lymphocyte act | 0.0 | Astrocytes rest | 13.8 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 40.9 |
| LAK cells IL-2 + IL-12 | 0.0 | Lupus kidney | 21.3 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 9.5 |
| PBMC PWM | 7.7 | Lung fibroblast TNF alpha + | 0.0 |
| | | IL-1 beta | |
| PBMC PHA-L | 5.8 | Lung fibroblast IL-4 | 0.0 |
| Ramos (B cell) none | 0.0 | Lung fibroblast IL-9 | 0.0 |
| Ramos (B cell) | 11.6 | Lung fibroblast IL-13 | 0.0 |
| ionomycin | | | |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 0.0 |
| | | TNF alpha | |
| EOL-1 dbcAMP | 5.9 | Dermal fibroblast CCD1070 | 0.0 |
| PMA/ionomycin | | IL-1 beta | |
| Dendritic cells none | 0.0 | Dermal fibroblast IFN | 0.0 |
| | | gamma | |
| Dendritic cells LPS | 4.0 | Dermal fibroblast IL-4 | 7.7 |
| Dendritic cells anti- | 0.0 | IBD Colitis 2 | 2.9 |
| CD40 | | | |
| Monocytes rest | 0.0 | IBD Crohn's | 0.0 |
| Monocytes LPS | 0.0 | Colon | 6.4 |
| Macrophages rest | 0.0 | Lung | 5.8 |
| Macrophages LPS | 0.0 | Thymus | 100.0 |
| HUVEC none | 0.0 | Kidney | 0.0 |
| HUVEC starved | 0.0 | | |

CNS_neurodegeneration_v1.0 Summary: Ag3272 Expression of the NOV22 gene is low/undetectable in all samples on this panel (CTs>35). (Data not shown.)

General_screening_panel_v1.4 Summary: Ag3272 Expression of the NOV22 gene is highest in a colon cancer cell line, SW-948 (CT=25.8). Moderate expression is also seen in two brain cancer cell lines and a lung cancer cell line. Thus, expression of this gene could be used to differentiate between these samples and other samples on this panel and as a marker to detect the presence of colon cancer. Furthermore, therapeutic modulation of the expression or function of this gene may be effective in the treatment of colon cancer.

In addition, this gene is expressed at much higher levels in fetal kidney (CT=32.6) than in adult kidney (CT=38). Thus, expression of this gene could be used to differentiate between adult and fetal sources of this tissue. Furthermore, expression of this gene in the fetal kidney suggests that this gene product may be involved in the development of this organ. Therefore, therapeutic modulation of the expression or function of this gene may be effective in the treatment of diseases of the kidney.

Panel 4D Summary: Ag3272 Significant expression of the NOV22 gene is restricted to the thymus (CT=34.1). Thus, the protein encoded by this gene may play an important role in T cell development and be a marker for this lymphoid tissue. Small molecule therapeutics, or antibody therapeutics designed against the protein encoded by this gene could be utilized to modulate immune function (T cell development) and be important for organ transplant, AIDS treatment or post chemotherapy immune reconstitution.

O. NOV27—CG5722801: Aldo-Keto Reductase Family 7, Member A3 Like Protein

Expression of the NOV27 gene was assessed using the primer-probe set Ag3143, described in Table OA. Results of the RTQ-PCR runs are shown in Tables OB, OC, OD and OE.

TABLE OA

Probe Name Ag3143

| | | | | |
|---|---|---|---|---|
| | | | | SEQ ID |
| Primers | Sequences | Length | Start Position | NO: |

| | | | | |
|---|---|---|---|---|
| Forward | 5′-ccctgaagcctgacagtgt-3′ | 19 | 308 | 515 |
| Probe | TET-5′-ctgcagtgtcccagagtggacctctt-3′-TAMRA | 26 | 358 | 516 |
| Reverse | 5′-tgtggtcaggtgcatgtagata-3′ | 22 | 385 | 517 |

TABLE OB
CNS_neurodegeneration_v1.0

| | Rel. Exp. (%) | | Rel. Exp. (%) |
|---|---|---|---|
| | Ag3143, Run | | Ag3143, Run |
| Tissue Name | 209057242 | Tissue Name | 209057242 |
| AD 1 Hippo | 27.4 | Control (Path) 3 | 13.5 |
| | | Temporal Ctx | |
| AD 2 Hippo | 57.0 | Control (Path) 4 | 42.9 |
| | | Temporal Ctx | |
| AD 3 Hippo | 20.2 | AD 1 Occipital Ctx | 30.8 |
| AD 4 Hippo | 13.3 | AD 2 Occipital Ctx | 0.0 |
| | | (Missing) | |
| AD 5 Hippo | 83.5 | AD 3 Occipital Ctx | 14.7 |
| AD 6 Hippo | 85.3 | AD 4 Occipital Ctx | 29.3 |
| Control 2 Hippo | 42.9 | AD 5 Occipital Ctx | 41.2 |
| Control 4 Hippo | 53.2 | AD 6 Occipital Ctx | 21.8 |
| Control (Path) 3 | 17.3 | Control 1 Occipital | 6.8 |
| Hippo | | Ctx | |
| AD 1 Temporal Ctx | 34.6 | Control 2 Occipital | 64.6 |
| | | Ctx | |
| AD 2 Temporal Ctx | 42.6 | Control 3 Occipital | 30.8 |
| | | Ctx | |
| AD 3 Temporal Ctx | 22.4 | Control 4 Occipital | 28.9 |
| | | Ctx | |
| AD 4 Temporal Ctx | 33.7 | Control (Path) 1 | 100.0 |
| | | Occipital Ctx | |
| AD 5 Inf Temporal | 99.3 | Control (Path) 2 | 18.9 |
| Ctx | | Occipital Ctx | |
| AD 5 Sup Temporal | 78.5 | Control (Path) 3 | 5.9 |
| Ctx | | Occipital Ctx | |
| AD 6 Inf Temporal | 56.3 | Control (Path) 4 | 23.5 |
| Ctx | | Occipital Ctx | |
| AD 6 Sup Temporal | 58.6 | Control 1 Parietal Ctx | 15.4 |
| Ctx | | | |
| Control 1 Temporal | 17.6 | Control 2 Parietal Ctx | 66.4 |
| Ctx | | | |
| Control 2 Temporal | 49.3 | Control 3 Parietal Ctx | 33.0 |
| Ctx | | | |
| Control 3 Temporal | 23.8 | Control (Path) 1 | 60.7 |
| Ctx | | Parietal Ctx | |
| Control 3 Temporal | 26.1 | Control (Path) 2 | 27.5 |
| Ctx | | Parietal Ctx | |
| Control (Path) 1 | 64.6 | Control (Path) 3 | 8.3 |
| Temporal Ctx | | Parietal Ctx | |
| Control (Path) 2 | 44.1 | Control (Path) 4 | 33.4 |
| Temporal Ctx | | Parietal Ctx | |

TABLE OC
Panel 1.3D

| | Rel. Exp. (%) | | Rel. Exp. (%) |
|---|---|---|---|
| | Ag3143, Run | | Ag3143, Run |
| Tissue Name | 167994819 | Tissue Name | 167994819 |
| Liver adenocarcinoma | 18.9 | Kidney (fetal) | 26.2 |
| Pancreas | 30.6 | Renal ca. 786-0 | 2.4 |
| Pancreatic ca. CAPAN2 | 6.4 | Renal ca. A498 | 8.5 |
| Adrenal gland | 4.7 | Renal ca. RXF 393 | 27.2 |
| Thyroid | 3.8 | Renal ca. ACHN | 8.5 |
| Salivary gland | 1.6 | Renal ca. UO-31 | 7.0 |
| Pituitary gland | 8.5 | Renal ca. TK-10 | 5.5 |
| Brain (fetal) | 15.3 | Liver | 66.0 |
| Brain (whole) | 13.3 | Liver (fetal) | 4.5 |
| Brain (amygdala) | 6.3 | Liver ca. (hepatoblast) | 19.1 |
| | | HepG2 | |
| Brain (cerebellum) | 19.6 | Lung | 3.0 |
| Brain (hippocampus) | 14.5 | Lung (fetal) | 8.8 |
| Brain (substantia nigra) | 12.6 | Lung ca. (small cell) | 18.0 |
| | | LX-1 | |
| Brain (thalamus) | 5.4 | Lung ca. (small cell) | 3.3 |
| | | NCI-H69 | |
| Cerebral Cortex | 19.5 | Lung ca. (s. cell var.) | 100.0 |
| | | SHP-77 | |
| Spinal cord | 9.5 | Lung ca. (large | 3.1 |
| | | cell) NCI-H460 | |
| glio/astro U87-MG | 15.0 | Lung ca. (non-sm. cell) | 16.3 |
| | | A549 | |
| glio/astro U-118-MG | 1.9 | Lung ca. (non-s. cell) | 10.6 |
| | | NCI-H23 | |
| astrocytoma SW1783 | 20.4 | Lung ca. (non-s. cell) | 0.0 |
| | | HOP-62 | |
| neuro*; met SK-N-AS | 14.5 | Lung ca. (non-s. cl) | 14.8 |
| | | NCI-H522 | |
| astrocytoma SF-539 | 5.8 | Lung ca. (squam.) SW | 4.9 |
| | | 900 | |
| astrocytoma SNB-75 | 17.0 | Lung ca. (squam.) | 4.6 |
| | | NCI-H596 | |
| glioma SNB-19 | 3.4 | Mammary gland | 14.7 |
| glioma U251 | 11.6 | Breast ca.* (pl. ef) | 10.9 |
| | | MCF-7 | |
| glioma SF-295 | 9.0 | Breast ca.* (pl. ef) | 7.9 |
| | | MDA-MB-231 | |
| Heart (fetal) | 52.1 | Breast ca.* (pl. ef) | 40.9 |
| | | T47D | |
| Heart | 2.4 | Breast ca. BT-549 | 5.7 |
| Skeletal muscle (fetal) | 16.5 | Breast ca. MDA-N | 7.5 |
| Skeletal muscle | 8.8 | Ovary | 14.9 |
| Bone marrow | 1.3 | Ovarian ca. OVCAR-3 | 7.8 |
| Thymus | 5.1 | Ovarian ca. OVCAR-4 | 9.5 |
| Spleen | 3.0 | Ovarian ca. OVCAR-5 | 29.7 |
| Lymph node | 2.8 | Ovarian ca. OVCAR-8 | 2.4 |
| Colorectal | 15.8 | Ovarian ca. IGROV-1 | 3.8 |
| Stomach | 32.3 | Ovarian ca.* (ascites) | 37.9 |
| | | SK-OV-3 | |
| Small intestine | 29.9 | Uterus | 4.2 |
| Colon ca. SW480 | 8.8 | Placenta | 1.0 |
| Colon ca.* | 41.2 | Prostate | 4.2 |
| SW620 (SW480 met) | | | |
| Colon ca. HT29 | 29.3 | Prostate ca.* (bone | 7.7 |
| | | met) PC-3 | |
| Colon ca. HCT-116 | 8.5 | Testis | 3.4 |
| Colon ca. CaCo-2 | 14.9 | Melanoma Hs688(A).T | 10.4 |
| Colon ca. | 9.2 | Melanoma* (met) | 2.2 |
| tissue (ODO3866) | | Hs688(B).T | |
| Colon ca. HCC-2998 | 26.6 | Melanoma UACC-62 | 5.8 |
| Gastric ca.* (liver met) | 9.5 | Melanoma M14 | 5.2 |
| NCI-N87 | | | |
| Bladder | 20.2 | Melanoma LOX IMVI | 6.5 |
| Trachea | 1.8 | Melanoma* (met) SK- | 6.3 |
| | | MEL-5 | |
| Kidney | 59.9 | Adipose | 4.5 |

TABLE OD
Panel 4D

| | Rel. Exp. (%) | | Rel. Exp. (%) |
|---|---|---|---|
| | Ag3143, Run | | Ag3143, Run |
| Tissue Name | 164527996 | Tissue Name | 164527996 |
| Secondary Th1 act | 3.2 | HUVEC IL-1beta | 1.5 |
| Secondary Th2 act | 2.7 | HUVEC IFN gamma | 3.6 |
| Secondary Tr1 act | 1.7 | HUVEC TNF alpha + IFN | 3.0 |
| | | gamma | |
| Secondary Th1 rest | 1.2 | HUVEC TNF alpha + IL4 | 5.8 |
| Secondary Th2 rest | 1.6 | HUVEC IL-11 | 4.3 |
| Secondary Tr1 rest | 2.3 | Lung Microvascular EC none | 4.5 |
| Primary Th1 act | 5.3 | Lung Microvascular EC | 5.7 |
| | | TNFalpha + IL-1beta | |
| Primary Th2 act | 4.9 | Microvascular Dermal EC | 6.2 |
| | | none | |
| Primary Tr1 act | 6.3 | Microsvasular Dermal EC | 5.4 |
| | | TNFalpha + IL-1beta | |
| Primary Th1 rest | 8.2 | Bronchial epithelium | 2.9 |
| | | TNFalpha + IL-1beta | |
| Primary Th2 rest | 5.9 | Small airway epithelium | 4.2 |
| | | none | |
| Primary Tr1 rest | 6.5 | Small airway epithelium | 11.0 |
| | | TNFalpha + IL-1beta | |
| CD45RA CD4 | 5.8 | Coronery artery SMC rest | 8.2 |
| lymphocyte act | | | |
| CD45RO CD4 | 5.7 | Coronery artery SMC | 4.8 |
| lymphocyte act | | TNFalpha + IL-1beta | |
| CD8 lymphocyte act | 7.3 | Astrocytes rest | 4.3 |
| Secondary CD8 | 5.4 | Astrocytes TNFalpha + IL- | 3.1 |
| lymphocyte rest | | 1beta | |
| Secondary CD8 | 6.3 | KU-812 (Basophil) rest | 7.4 |
| lymphocyte act | | | |
| CD4 lymphocyte none | 2.6 | KU-812 (Basophil) | 6.3 |
| | | PMA/ionomycin | |
| 2ry Th1/Th2/Tr1_anti- | 5.5 | CCD1106 (Keratinocytes) | 4.4 |
| CD95 CH11 | | none | |
| LAK cells rest | 12.4 | CCD1106 (Keratinocytes) | 1.5 |
| | | TNFalpha + IL-1beta | |
| LAK cells IL-2 | 10.3 | Liver cirrhosis | 18.0 |
| LAK cells IL-2 + IL-12 | 6.3 | Lupus kidney | 2.8 |
| LAK cells IL-2 + IFN | 7.1 | NCI-H292 none | 10.9 |
| gamma | | | |
| LAK cells IL-2 + IL-18 | 5.9 | NCI-H292 IL-4 | 11.0 |
| LAK cells | 2.9 | NCI-H292 IL-9 | 11.6 |
| PMA/ionomycin | | | |
| NK Cells IL-2 rest | 2.9 | NCI-H292 IL-13 | 6.3 |
| Two Way MLR 3 day | 4.3 | NCI-H292 IFN gamma | 7.1 |
| Two Way MLR 5 day | 4.7 | HPAEC none | 3.8 |
| Two Way MLR 7 day | 3.8 | HPAEC TNFalpha + IL-1 | 3.7 |
| | | beta | |
| PBMC rest | 3.2 | Lung fibroblast none | 2.9 |
| PBMC PWM | 11.8 | Lung fibroblast TNF alpha + | 1.6 |
| | | IL-1 beta | |
| PBMC PHA-L | 7.7 | Lung fibroblast IL-4 | 7.1 |
| Ramos (B cell) none | 6.5 | Lung fibroblast IL-9 | 4.6 |
| Ramos (B cell) | 31.2 | Lung fibroblast IL-13 | 5.8 |
| ionomycin | | | |
| B lymphocytes PWM | 18.3 | Lung fibroblast IFN gamma | 5.8 |
| B lymphocytes CD40L | 17.1 | Dermal fibroblast CCD1070 | 12.9 |
| and IL-4 | | rest | |
| EOL-1 dbcAMP | 12.8 | Dermal fibroblast CCD1070 | 12.9 |
| | | TNF alpha | |
| EOL-1 dbcAMP | 4.6 | Dermal fibroblast CCD1070 | 3.9 |
| PMA/ionomycin | | IL-1 beta | |
| Dendritic cells none | 4.7 | Dermal fibroblast IFN | 7.8 |
| | | gamma | |
| Dendritic cells LPS | 2.1 | Dermal fibroblast IL-4 | 15.2 |
| Dendritic cells anti- | 4.8 | IBD Colitis 2 | 0.8 |
| CD40 | | | |
| Monocytes rest | 3.5 | IBD Crohn's | 28.9 |
| Monocytes LPS | 0.4 | Colon | 98.6 |
| Macrophages rest | 10.8 | Lung | 6.3 |
| Macrophages LPS | 3.1 | Thymus | 100.0 |
| HUVEC none | 5.8 | Kidney | 13.1 |
| HUVEC starved | 12.8 | | |

TABLE OE
Panel 5 Islet

| | Rel. Exp. (%) | | Rel. Exp. (%) |
|---|---|---|---|
| | Ag3143, Run | | Ag3143, Run |
| Tissue Name | 233698023 | Tissue Name | 233698023 |
| 97457_Patient- | 23.7 | 94709_Donor 2 AM - A_adipose | 63.3 |
| 02go_adipose | | | |
| 97476_Patient- | 11.2 | 94710_Donor 2 AM - B_adipose | 26.1 |
| 07sk_skeletal muscle | | | |
| 97477_Patient- | 18.4 | 94711_Donor 2 AM - C_adipose | 19.2 |
| 07ut_uterus | | | |
| 97478_Patient- | 13.1 | 94712_Donor 2 AD - A_adipose | 54.0 |
| 07pl_placenta | | | |
| 99167_Bayer Patient 1 | 72.7 | 94713_Donor 2 AD - B_adipose | 71.7 |
| 97482_Patient- | 10.0 | 94714_Donor 2 AD - C_adipose | 77.9 |
| 08ut_uteras | | | |
| 97483_Patient- | 11.0 | 94742_Donor 3 U- | 7.3 |
| 08pl_placenta | | A_Mesenchymal Stem Cells | |
| 97486_Patient- | 8.1 | 94743_Donor 3 U- | 6.3 |
| 09sk_skeletal muscle | | B_Mesenchymal Stem Cells | |
| 97487_Patient- | 11.3 | 94730_Donor 3 AM - A_adipose | 10.6 |
| 09ut_uterus | | | |
| 97488_Patient- | 12.8 | 94731_Donor 3 AM - B_adipose | 6.5 |
| 09pl_placenta | | | |
| 97492_Patient- | 26.2 | 94732_Donor 3 AM - C_adipose | 6.3 |
| 10ut_uterus | | | |
| 97493_Patient- | 48.6 | 94733_Donor 3 AD - A_adipose | 11.0 |
| 10pl_placenta | | | |
| 97495_Patient- | 8.5 | 94734_Donor 3 AD - B_adipose | 7.0 |
| 11go_adipose | | | |
| 97496_Patient- | 14.7 | 94735_Donor 3 AD - C_adipose | 3.4 |
| 11sk_skeletal muscle | | | |
| 97497_Patient- | 22.4 | 77138_Liver_HepG2untreated | 46.3 |
| 11ut_uterus | | | |
| 97498_Patient- | 33.9 | 73556_Heart_Cardiac stromal cells | 6.2 |
| 11pl_placenta | | (primary) | |
| 97500_Patient- | 30.6 | 81735_Small Intestine | 76.8 |
| 12go_adipose | | | |
| 97501_Patient- | 50.3 | 72409_Kidney_Proximal | 9.2 |
| 12sk_skeletal muscle | | Convoluted Tubule | |
| 97502_Patient- | 9.0 | 82685_Small intestine_Duodenum | 100.0 |
| 12ut_uterus | | | |
| 97503_Patient- | 3.9 | 90650_Adrenal_Adrenocortical | 6.2 |
| 12pl_placenta | | adenoma | |
| 94721_Donor2U- | 45.4 | 72410_Kidney_HRCE | 27.0 |
| A_Mesenchymal Stem | | | |
| Cells | | | |
| 94722_Donor 2 U- | 39.8 | 72411_Kidney_HRE | 8.7 |
| B_Mesenchymal Stem | | | |
| Cells | | | |
| 94723_Donor 2 U- | 64.2 | 73139_Uterus_Uterine smooth | 15.9 |
| C_Mesenchymal Stem | | muscle cells | |
| Cells | | | |

CNS_neurodegeneration_v1.0 Summary: Ag3143 This panel does not show differential expression of the NOV27 gene in Alzheimer's disease. However, this expression profile confirms the presence of this gene in the brain. Please see Panel 1.3D for discussion of utility of this gene in the central nervous system.

Panel 1.3D Summary: Ag3413 The NOV27 gene is expressed at a low level in most of the cancer cell lines and normal tissues on this panel. There appears to be significantly higher expression in lung, breast and ovarian cancer cell lines. Thus, therapeutic inhibition of this gene product, through the use of small molecule drugs, might be of utility in the treatment of the above listed cancer types.

Among metabolic tissues, this gene has low levels of expression (CT values=31-34) in pancreas, pituitary, skeletal muscle and liver. This aldoketoreductase may be a small molecule target for the treatment of endocrine and metabolic disease, including Types 1 and 2 diabetes and obesity. In addition, this gene appears to be differentially expressed in fetal (CT value=32) vs adult heart (CT value=36) and may be useful for the identification of the fetal phenotype in this tissue. It also appears to be differentially expressed in adult (CT value=31) vs fetal liver (CT value=35) and may also be useful for the identification of the adult phenotype in this tissue.

In addition, low expression throughout the brain suggests a role for this gene in CNS processes. Members of the aldo-keto reductase superfamily are known to function in the processing of hormones in the brain. Brain hormone regulation mediates numerous clinically significant conditions, including psychiatric disorders such as anxiety, overeating and memory disorders. Therefore, agents that modulate the activity of this gene product have potential utility in the treatment of these disorders.

### REFERENCES

Penning T M, Burczynski M E, Jez J M, Hung C F, Lin H K, Ma H, Moore M, Palackal N, Ratnam K. Human 3alpha-hydroxysteroid dehydrogenase isoforms (AKR1C1-AKR1C4) of the aldo-keto reductase superfamily: functional plasticity and tissue distribution reveals roles in the inactivation and formation of male and female sex hormones. Biochem J Oct. 1, 2000;351(Pt 1):67-77

The kinetic parameters, steroid substrate specificity and identities of reaction products were determined for four homogeneous recombinant human 3alpha-hydroxysteroid dehydrogenase (3alpha-HSD) isoforms of the aldo-keto reductase (AKR) superfamily. The enzymes correspond to type 13alpha-HSD (AKRIC4), type 23alpha(17beta)-HSD (AKR1C3), type 33alpha-HSD (AKR1C2) and 20alpha(3alpha)-HSD (AKR1C1), and share at least 84% amino acid sequence identity. All enzymes acted as NAD(P)(H)-dependent 3-, 17- and 20-ketosteroid reductases and as 3alpha-, 17beta- and 20alpha-hydroxysteroid oxidases. The functional plasticity of these isoforms highlights their ability to modulate the levels of active androgens, oestrogens and progestins. Salient features were that AKR1C4 was the most catalytically efficient, with k(cat)/K(m) values for substrates that exceeded those obtained with other isoforms by 10-30-fold. In the reduction direction, all isoforms inactivated 5alpha-dihydrotestosterone (17beta-hydroxy-5alpha-androstan-3-one; 5alpha-DHT) to yield 5alpha-androstane-3alpha,17beta-diol (3alpha-androstanediol). However, only AKR1C3 reduced Delta(4)-androstene-3,17-dione to produce significant amounts of testosterone. All isoforms reduced oestrone to 17beta-oestradiol, and progesterone to 20alpha-hydroxy-pregn-4-ene-3,20-dione (20alpha-hydroxyprogesterone). In the oxidation direction, only AKR1C2 converted 3alpha-androstanediol to the active hormone 5alpha-DHT. AKR1C3 and AKR1C4 oxidized testosterone to Delta(4)-androstene-3,17-dione. All isoforms oxidized 17beta-oestradiol to oestrone, and 20alpha-hydroxyprogesterone to progesterone. Discrete tissue distribution of these AKRIC enzymes was observed using isoform-specific reverse transcriptase-PCR. AKR1C4 was virtually liver-specific and its high k(cat)/K(m) allows this enzyme to form 5alpha/5beta-tetrahydrosteroids robustly. AKR1C3 was most prominent in the prostate and mammary glands. The ability of AKR1C3 to interconvert testosterone with Delta(4)-androstene-3,17-dione, but to inactivate 5alpha-DHT, is consistent with this enzyme eliminating active androgens from the prostate. In the mammary gland, AKR1C3 will convert Delta(4)-androstene-3,17-dione to testosterone (a substrate aromatizable to 17beta-oestradiol), oestrone to 17beta-oestradiol, and progesterone to 20alpha-hydroxyprogesterone, and this concerted reductive activity may yield a pro-oesterogenic state. AKR1C3 is also the dominant form in the uterus and is responsible for the synthesis of 3alpha-androstanediol which has been implicated as a parturition hormone. The major isoforms in the brain, capable of synthesizing anxiolytic steroids, are AKR1C1 and AKR1C2. These studies are in stark contrast with those in rat where only a single AKR with positional- and stereo-specificity for 3alpha-hydroxysteroids exists. [egunther, 29-Jan-02]

Panel 4D Summary: Ag3143 The NOV27 gene is expressed at high to moderate levels in a wide range of cell types of significance in the immune response and tissue response in health and disease, with the highest expression being detected colon and thymus (CT=28.1). Therefore, targeting of this gene product with a small molecule drug or antibody therapeutic may modulate the functions of cells of the immune system as well as resident tissue cells and lead to improvement of the symptoms of patients suffering from autoimmune and inflammatory diseases such as COPD, emphysema, asthma, allergies, inflammatory bowel disease, lupus erythematosus, and arthritis, including osteoarthritis and rheumatoid arthritis

Panel 5 Islet Summary: Ag3143 The NOV27 gene has low levels of expression in adipose, skeletal muscle and Islets of Langerhans. It is also expressed at low levels in mesenchymal stem cells that can be differentiated in vitro into adipocytes, chondrocytes and osteocytes. Therefore, this gene product may a small molecule target for the treatment of diseases of bone and cartilage and adipose.

P. NOV25—CG57276-01: Endolyn Precursor-Like Protein

Expression of the NOV25 gene was assessed using the primer-probe set Ag3149, described in Table PA.

TABLE PA

Probe Name Ag3149

| | | | | |
|---|---|---|---|---|
| | | | | SEQ ID |
| Primers | Sequences | Length | Start Position | NO: |

| | | | | |
|---|---|---|---|---|
| Forward | 5′-ccccttctacaacttccaagac-3′ | 22 | 468 | 518 |
| Probe | TET-5′-caacaaataacactgtgactccaacctca-3′-TAMRA | 29 | 507 | 519 |
| Reverse | 5′-aaggtagactttcgcacaggtt-3′ | 22 | 537 | 520 |

CNS_neurodegeneration_v1.0 Summary: Ag3149 Expression of the NOV25 gene is low/undetectable in all samples on this panel (CTs>35). (Data not shown.)

Panel 1.3D Summary: Ag3149 Expression of the NOV25 gene is low/undetectable in all samples on this panel (CTs>35). (Data not shown.)

Panel 4D Summary: Ag3149 Expression of the NOV25 gene is low/undetectable in all samples on this panel (CTs>35). (Data not shown.)

Q. NOV26—CG57224-01: Arylacetamide Deacetylase

Expression of the NOV26 gene was assessed using the primer-probe set Ag3136, described in Table QA. Results of the RTQ-PCR runs are shown in Tables QB and QC.

TABLE QA

Probe Name Ag3136

| | | | | |
|---|---|---|---|---|
| | | | | SEQ ID |
| Primers | Sequences | Length | Start Position | NO: |

| | | | | |
|---|---|---|---|---|
| Foward | 5′-cccagtttccactcactcatta-3′ | 22 | 203 | 521 |
| Probe | TET-5′-acagtgctcttggccctgcatgt-3′-TAMRA | 23 | 226 | 522 |
| Reverse | 5′-acaggatatagaccccaaatgg-3′ | 122 | 259 | 523 |

TABLE QB
Panel 1.3D

| | Rel. Exp. (%) | | Rel. Exp. (%) |
|---|---|---|---|
| | Ag3136, Run | | Ag3136, Run |
| Tissue Name | 167994424 | Tissue Name | 167994424 |
| Liver adenocarcinoma | 11.6 | Kidney (fetal) | 24.7 |
| Pancreas | 2.8 | Renal ca. 786-0 | 3.7 |
| Pancreatic ca. CAPAN2 | 15.6 | Renal ca. A498 | 5.9 |
| Adrenal gland | 1.4 | Renal ca. RXF 393 | 0.7 |
| Thyroid | 5.0 | Renal ca. ACHN | 7.3 |
| Salivary gland | 4.3 | Renal ca. UO-31 | 1.3 |
| Pituitary gland | 1.4 | Renal ca. TK-10 | 3.8 |
| Brain (fetal) | 0.7 | Liver | 4.5 |
| Brain (whole) | 1.7 | Liver (fetal) | 2.8 |
| Brain (amygdala) | 0.5 | Liver ca. (hepatoblast) | 16.0 |
| | | HepG2 | |
| Brain (cerebellum) | 0.6 | Lung | 6.2 |
| Brain (hippocampus) | 0.7 | Lung (fetal) | 7.2 |
| Brain (substantia nigra) | 2.7 | Lung ca. (small cell) | 4.8 |
| | | LX-1 | |
| Brain (thalamus) | 1.4 | Lung ca. (small cell) | 3.7 |
| | | NCI-H69 | |
| Cerebral Cortex | 0.8 | Lung ca. (s. cell var.) | 1.6 |
| | | SHP-77 | |
| Spinal cord | 6.0 | Lung ca. (large | 3.1 |
| | | cell) NCI-H460 | |
| glio/astro U87-MG | 7.5 | Lung ca. (non-sm. cell) | 18.0 |
| | | A549 | |
| glio/astro U-118-MG | 0.0 | Lung ca (non-s. cell) | 1.4 |
| | | NCI-H23 | |
| astrocytoma SW1783 | 1.6 | Lung ca. (non-s. cell) | 3.9 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) SW | 63.3 |
| | | 900 | |
| astrocytoma SNB-75 | 32.8 | Lung ca. (squam.) | 2.5 |
| | | NCI-H596 | |
| glioma SNB-19 | 1.6 | Mammary gland | 10.8 |
| glioma U251 | 19.2 | Breast ca.* (pl. ef) | 0.3 |
| | | MCF-7 | |
| glioma SF-295 | 13.9 | Breast ca.* (pl. ef) | 0.0 |
| | | MDA-MB-231 | |
| Heart (fetal) | 2.1 | Breast ca.* (pl. ef) | 3.5 |
| | | T47D | |
| Heart | 3.1 | Breast ca. BT-549 | 2.5 |
| Skeletal muscle (fetal) | 7.0 | Breast ca. MDA-N | 1.0 |
| Skeletal muscle | 1.0 | Ovary | 0.6 |
| Bone marrow | 4.0 | Ovarian ca. OVCAR-3 | 0.9 |
| Thymus | 3.0 | Ovarian ca. OVCAR-4 | 0.0 |
| Spleen | 1.6 | Ovarian ca. OVCAR-5 | 100.0 |
| Lymph node | 6.4 | Ovarian ca. OVCAR-8 | 0.6 |
| Colorectal | 0.8 | Ovarian ca. IGROV-1 | 0.4 |
| Stomach | 5.1 | Ovarian ca.* (ascites) | 1.3 |
| | | SK-OV-3 | |
| Small intestine | 4.1 | Uterus | 5.7 |
| Colon ca. SW480 | 0.7 | Placenta | 1.7 |
| Colon ca.* | 2.0 | Prostate | 12.4 |
| SW620 (SW480 met) | | | |
| Colon ca. HT29 | 3.5 | Prostate ca.* (bone | 4.4 |
| | | met) PC-3 | |
| Colon ca. HCT-116 | 3.1 | Testis | 0.3 |
| Colon ca. CaCo-2 | 6.5 | Melanoma Hs688(A).T | 3.2 |
| Colon ca. | 0.0 | Melanoma* (met) | 1.0 |
| tissue (ODO3866) | | Hs688(B).T | |
| Colon ca. HCC-2998 | 2.5 | Melanoma UACC-62 | 10.0 |
| Gastric ca.* (liver met) | 21.6 | Melanoma M14 | 2.4 |
| NCI-N87 | | | |
| Bladder | 11.5 | (Melanoma LOX IMVI | 5.8 |
| Trachea | 4.6 | Melanoma* (met) SK- | 0.0 |
| | | MEL-5 | |
| Kidney | 8.4 | Adipose | 4.2 |

TABLE QC
Panel 4D

| | Rel. Exp. (%) | | Rel. Exp. (%) |
|---|---|---|---|
| | Ag3136, Run | | Ag3136, Run |
| Tissue Name | 164527948 | Tissue Name | 164527948 |
| Secondary Th1 act | 4.1 | HUVEC IL-1beta | 1.8 |
| Secondary Th2 act | 11.0 | HUVEC IFN gamma | 8.8 |
| Secondary Tr1 act | 15.3 | HUVEC TNF alpha + IFN | 3.5 |
| | | gamma | |
| Secondary Th1 rest | 4.8 | HUVEC TNF alpha + IL4 | 1.3 |
| Secondary Th2 rest | 10.1 | HUVEC IL-11 | 9.2 |
| Secondary Tr1 rest | 7.9 | Lung Microvascular EC none | 3.6 |
| Primary Th1 act | 32.1 | Lung Microvascular EC | 7.4 |
| | | TNFalpha + IL-1beta | |
| Primary Th2 act | 22.5 | Microvascular Dermal EC | 15.2 |
| | | none | |
| Primary Tr1 act | 40.1 | Microsvasular Dermal EC | 6.9 |
| | | TNFalpha + IL-1beta | |
| Primary Th1 rest | 97.3 | Bronchial epithelium | 3.4 |
| | | TNFalpha + IL-1beta | |
| Primary Th2 rest | 41.5 | Small airway epithelium | 1.8 |
| | | none | |
| Primary Tr1 rest | 54.7 | Small airway epithelium | 6.3 |
| | | TNFalpha + IL-1beta | |
| CD45RA CD4 | 12.1 | Coronery artery SMC rest | 30.1 |
| lymphocyte act | | | |
| CD45RO CD4 | 13.6 | Coronery artery SMC | 4.6 |
| lymphocyte act | | TNFalpha + IL-1beta | |
| CD8 lymphocyte act | 20.4 | Astrocytes rest | 6.0 |
| Secondary CD8 | 4.9 | Astrocytes TNFalpha + IL- | 0.0 |
| lymphocyte rest | | 1beta | |
| Secondary CD8 | 7.9 | KU-812 (Basophil) rest | 24.8 |
| lymphocyte act | | | |
| CD4 lymphocyte none | 37.4 | KU-812 (Basophil) | 80.7 |
| | | PMA/ionomycin | |
| 2ry Th1/Th2/Tr1_anti- | 13.6 | CCD1106 (Keratinocytes) | 1.7 |
| CD95 CH11 | | none | |
| LAK cells rest | 14.1 | CCD1106 (Keratinocytes) | 0.0 |
| | | TNFalpha + IL-1beta | |
| LAK cells IL-2 | 13.0 | Liver cirrhosis | 11.4 |
| LAK cells IL-2 + IL-12 | 16.2 | Lupus kidney | 2.6 |
| LAK cells IL-2 + IFN | 45.4 | NCI-H292 none | 100.0 |
| gamma | | | |
| LAK cells IL-2 + IL-18 | 39.8 | NCI-H292 IL-4 | 57.0 |
| LAK cells | 7.0 | NCI-H292 IL-9 | 54.0 |
| PMA/ionomycin | | | |
| NK Cells IL-2 rest | 49.3 | NCI-H292 IL-13 | 28.3 |
| Two Way MLR 3 day | 13.8 | NCI-H292 IFN gamma | 35.1 |
| Two Way MLR 5 day | 13.1 | HPAEC none | 5.8 |
| Two Way MLR 7 day | 9.8 | HPAEC TNF alpha + IL-1 | 2.0 |
| | | beta | |
| PBMC rest | 13.7 | Lung fibroblast none | 0.7 |
| PBMC PWM | 46.3 | Lung fibroblast TNF alpha + | 0.0 |
| | | IL-1 beta | |
| PBMC PHA-L | 27.7 | Lung fibroblast IL-4 | 0.9 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 22.1 | Lung fibroblast IFN gamma | 0.6 |
| B lymphocytes CD40L | 8.7 | Dermal fibroblast CCD1070 | 6.0 |
| and IL-4 | | rest | |
| EOL-1 dbcAMP | 0.0 | Dermal fibroblast CCD1070 | 14.0 |
| | | TNF alpha | |
| EOL-1 dbcAMP | 3.7 | Dermal fibroblast CCD1070 | 7.3 |
| PMA/ionomycin | | IL-1 beta | |
| Dendritic cells none | 7.5 | Dermal fibroblast IFN | 1.3 |
| | | gamma | |
| Dendritic cells LPS | 9.5 | Dermal fibroblast IL-4 | 6.0 |
| Dendritic cells anti- | 18.7 | IBD Colitis 2 | 0.6 |
| CD40 | | | |
| Monocytes rest | 0.0 | IBD Crohn's | 4.0 |
| Monocytes LPS | 0.5 | Colon | 26.6 |
| Macrophages rest | 27.0 | Lung | 9.5 |
| Macrophages LPS | 6.3 | Thymus | 82.4 |
| HUVEC none | 10.4 | Kidney | 27.2 |
| HUVEC starved | 10.9 | | |

Panel 1.3D Summary: Ag3136 Expression of the NOV26 gene is widespread throughout this panel, with highest expression in an ovarian cancer cell line (CT=31). Significant levels of expression are also seen in cell lines derived from lung, gastric, and brain cancers. Thus, expression of the NOV26 gene could be used to differentiate between these samples and other samples on this panel and as a marker to detect the presence of these cancers. Furthermore, therapeutic modulation of the expression or function of this gene may be effective in the treatment of lung, gastric, and brain cancers.

Panel 4D Summary: Ag3136 Highest expression of the NOV26 gene is seen in untreated NCI-H292 cells (CT=29). Significant levels of expression are also seen in a cluster of treated cells derived from the NCI-H292 cells, a human airway epithelial cell line that produces mucins. Mucus overproduction is an important feature of bronchial asthma and chronic obstructive pulmonary disease samples. The NOV26 transcript is also expressed at lower but still significant levels in small airway epithelium treated with IL-1 beta and TNF-alpha. The expression of the transcript in this mucoepidermoid cell line that is often used as a model for airway epithelium (NCI-H292 cells) suggests that this transcript may be important in the proliferation or activation of airway epithelium. Therefore, therapeutics designed with the protein encoded by the transcript may reduce or eliminate symptoms caused by inflammation in lung epithelia in chronic obstructive pulmonary disease, asthma, allergy, and emphysema.

In addition, this transcript is induced in the PMA and ionomycin treated basophil cell line KU-812. Basophils release histamines and other biological modifiers in response to allergens and play an important role in the pathology of asthma and hypersensitivity reactions. Therefore, therapeutics designed against the putative protein encoded by this gene may reduce or inhibit inflammation by blocking basophil function in these diseases. In addition, these cells are a reasonable model for the inflammatory cells that take part in various inflammatory lung and bowel diseases, such as asthma, Crohn's disease, and ulcerative colitis. Therefore, therapeutics that modulate the function of this gene product may reduce or eliminate the symptoms of patients suffering from asthma, Crohn's disease, and ulcerative colitis.

R. NOV28—CG57213-01: PB39

Expression of the NOV28 gene was assessed using the primer-probe set Ag4870, described in Table RA. Results of the RTQ-PCR runs are shown in Table RB.

TABLE RA

Probe Name Ag4870

| | | | | |
|---|---|---|---|---|
| Primers | Sequences | Length | Start Position | SEQ ID NO: |

| | | | | |
|---|---|---|---|---|
| Forward | 5′-gcctgccttatctttctgaact-3′ | 22 | 599 | 524 |
| Probe | TET-5′-ctttcctgcccctgaggaagtcaatt-3′-TAMRA | 26 | 646 | 525 |
| Reverse | 5′-cactcagcttgatcttcttcgt-3′ | 22 | 674 | 526 |

TABLE RB
General_screening_panel_v1.5

| | Rel. Exp. (%) | | Rel. Exp. (%) |
|---|---|---|---|
| | Ag4870, Run | | Ag4870, Run |
| Tissue Name | 228903631 | Tissue Name | 228903631 |
| Adipose | 2.0 | Renal ca. TK-10 | 31.4 |
| Melanoma* | 6.0 | Bladder | 26.6 |
| Hs688(A).T | | | |
| Melanoma* | 4.2 | Gastric ca. (liver met.) | 7.4 |
| Hs688(B).T | | NCI-N87 | |
| Melanoma* M14 | 20.4 | Gastric ca. KATO III | 66.0 |
| Melanoma* LOX IMVI | 0.9 | Colon ca. SW-948 | 10.2 |
| Melanoma* SK-MEL- | 7.8 | Colon ca. SW480 | 16.8 |
| 5 | | | |
| Squamous cell | 0.5 | Colon ca.* (SW480 met) | 63.7 |
| carcinoma SCC-4 | | SW620 | |
| Testis Pool | 1.3 | Colon ca. HT29 | 17.1 |
| Prostate ca.* (bone | 24.7 | Colon ca. HCT-116 | 4.4 |
| met) PC-3 | | | |
| Prostate Pool | 4.0 | Colon ca. CaCo-2 | 36.6 |
| Placenta | 3.5 | Colon cancer tissue | 4.0 |
| Uterus Pool | 5.0 | Colon ca. SW1116 | 5.7 |
| Ovarian ca. OVCAR-3 | 0.8 | Colon ca. Colo-205 | 6.7 |
| Ovarian ca. SK-OV-3 | 1.2 | Colon ca. SW-48 | 17.1 |
| Ovarian ca. OVCAR-4 | 0.5 | Colon Pool | 6.3 |
| Ovarian ca. OVCAR-5 | 17.1 | Small Intestine Pool | 5.0 |
| Ovarian ca. IGROV-1 | 2.8 | Stomach Pool | 5.4 |
| Ovarian ca. OVCAR-8 | 5.8 | Bone Marrow Pool | 2.3 |
| Ovary | 7.5 | Fetal Heart | 1.0 |
| Breast ca. MCF-7 | 1.9 | Heart Pool | 3.3 |
| Breast ca. MDA-MB- | 8.5 | Lymph Node Pool | 6.1 |
| 231 | | | |
| Breast ca. BT 549 | 3.2 | Fetal Skeletal Muscle | 2.5 |
| Breast ca. T47D | 0.4 | Skeletal Muscle Pool | 9.0 |
| Breast ca. MDA-N | 15.6 | Spleen Pool | 1.8 |
| Breast Pool | 8.0 | Thymus Pool | 4.2 |
| Trachea | 7.3 | CNS cancer (glio/astro) | 1.6 |
| | | U87-MG | |
| Lung | 1.5 | CNS cancer (glio/astro) | 0.9 |
| | | U-118-MG | |
| Fetal Lung | 11.6 | CNS cancer (neuro; met) | 7.0 |
| | | SK-N-AS | |
| Lung ca. NCI-N417 | 1.6 | CNS cancer (astro) SF- | 3.1 |
| | | 539 | |
| Lung ca. LX-1 | 49.3 | CNS cancer (astro) SNB- | 14.9 |
| | | 75 | |
| Lung ca. NCI-H146 | 2.4 | CNS cancer (glio) SNB- | 4.2 |
| | | 19 | |
| Lung ca. SHP-77 | 3.5 | CNS cancer (glio) SF- | 10.0 |
| | | 295 | |
| Lung ca. A549 | 1.6 | Brain (Amygdala) Pool | 0.5 |
| Lung ca. NCI-H526 | 0.2 | Brain (cerebellum) | 1.1 |
| Lung ca. NCI-H23 | 0.8 | Brain (fetal) | 1.0 |
| Lung ca. NCI-H460 | 1.2 | Brain (Hippocampus) | 0.4 |
| | | Pool | |
| Lung ca. HOP-62 | 5.3 | Cerebral Cortex Pool | 0.4 |
| Lung ca. NCI-H522 | 6.5 | Brain (Substantia nigra) | 0.3 |
| | | Pool | |
| Liver | 21.6 | Brain (Thalamus) Pool | 0.6 |
| Fetal Liver | 100.0 | Brain (whole) | 3.0 |
| Liver ca. HepG2 | 68.3 | Spinal Cord Pool | 0.4 |
| Kidney Pool | 9.3 | Adrenal Gland | 8.1 |
| Fetal Kidney | 1.8 | Pituitary gland Pool | 0.3 |
| Renal ca. 786-0 | 0.5 | Salivary Gland | 9.3 |
| Renal ca. A498 | 0.1 | Thyroid (female) | 1.7 |
| Renal ca. ACHN | 0.1 | Pancreatic ca. CAPAN2 | 1.4 |
| Renal ca. UO-31 | 2.3 | Pancreas Pool | 16.6 |

General_screening_panel_v1.5 Summary: Ag4870 Highest expression of the NOV28 gene, a PB39 homolog, is seen in the fetal liver. Significant levels of expression are also seen in cell lines derived from lung, gastric, colon, renal, liver, ovarian, breast, prostate, melanoma and brain cancers. This expression in proliferative samples suggests a role for the NOV28 gene in cell proliferation and growth. This is consistent with data that shows to be upregulated in prostate cancer and tissues undergoing growth and differentiation. Thus, expression of this gene could be used to differentiate between these samples and other samples on this panel and as a marker to detect the presence of these cancers. Furthermore, therapeutic modulation of the expression or function of this gene may be effective in the treatment of these cancers.

### REFERENCES

Cole K A, Chuaqui R F, Katz K, Pack S, Zhuang Z, Cole C E, Lyne J C, Linehan W M, Liotta L A, Emmert-Buck M R. cDNA sequencing and analysis of POV1 (PB39): a novel gene up-regulated in prostate cancer. Genomics Jul. 15, 1998;51(2):282-7

We recently identified a novel gene (PB39) (HGMW-approved symbol POVI) whose expression is up-regulated in human prostate cancer using tissue microdissection-based differential display analysis. In the present study we report the full-length sequencing of PB39 cDNA, genomic localization of the PB39 gene, and genomic sequence of the mouse homologue. The full-length human cDNA is 2317 nucleotides in length and contains an open reading frame of 559 amino acids which does not show homology with any reported human genes. The N-terminus contains charged amino acids and a helical loop pattern suggestive of an srp leader sequence for a secreted protein. Fluorescence in situ hybridization using PB39 cDNA as probe mapped the gene to chromosome 11p11.1-p11.2. Comparison of PB39 cDNA sequence with murine sequence available in the public database identified a region of previously sequenced mouse genomic DNA showing 67% amino acid sequence homology with human PB39. Based on alignment and comparison to the human cDNA the mouse genomic sequence suggests there are at least 14 exons in the mouse gene spread over approximately 100 kb of genomic sequence. Further analysis of PB39 expression in human tissues shows the presence of a unique splice variant mRNA that appears to be primarily associated with fetal tissues and tumors. Interestingly, the unique splice variant appears in prostatic intraepithelial neoplasia, a microscopic precursor lesion of prostate cancer. The current data support the hypothesis that PB39 plays a role in the development of human prostate cancer and will be useful in the analysis of the gene product in further human and murine studies.

PMID: 9722952

Stuart R O, Pavlova A, Beier D, Li Z, Krijanovski Y, Nigam SK. EEG1, a putative transporter expressed during epithelial organogenesis: comparison with embryonic transporter expression during nephrogenesis. Am J Physiol Renal Physiol December 2001;281(6):F1148-56

A screen for genes differentially regulated in a model of kidney development identified the novel gene embryonic epithelia gene 1 (EEG1). EEG1 exists as two transcripts of 2.4 and 3.5 kb that are most highly expressed at embryonic day 7 and later in the fetal liver, lung, placenta, and kidney. The EEG1 gene is composed of 14 exons spanning a 20-kb region at human chromosome 11p12 and the syntenic region of mouse chromosome 2. Six EEG1 exons have previously been assigned to a longer isoform of eosinophil major basic protein termed proteoglycan 2. Another gene distantly related to EEG1, POV1/PB39, is located 88 kb upstream from the EEG1 gene on chromosome 11. Temporal expression of 65 members of the solute carrier (SLC)-class of transport proteins was followed during kidney development using DNA arrays. POV-1 and EEG1, like glucose transporters, displayed very early maximal gene expression. In contrast, other SLC genes, such as organic anion and cation transporters, amino acid permneases, and nucleoside transporters, had maximal expression later in development. Thus, although the bulk of transporters are expressed late in kidney development, a fraction are expressed near the onset of nephrogenesis. The data raise the possibility that EEG1 and POV1 may define a new family of transport proteins involved in the transport of nutrients or metabolites in rapidly growing and/or developing tissues.

PMID: 11704567

S. NOV31—CG57344-01 and CG57344-02: Myelin P2-Like

Expression of NOV31 gene was assessed using the primer-probe set Ag3205, described in Table SA. Results of the RTQ-PCR runs are shown in Tables SB and SC.

TABLE SA

Probe Name Ag3205

| | | | | |
|---|---|---|---|---|
| | | | | SEQ ID |
| Primers | Sequences | Length | Start Position | NO: |

| | | | | |
|---|---|---|---|---|
| Forward | 5′-accagctccaaggaacatg-3′ | 19 | 28 | 527 |
| Probe | TET-5′-tccatttcttgtgaaaattccgaaga-3′-TAMRA | 26 | 51 | 528 |
| Reverse | 5′-ttcctatacccagctccttca-3′ | 21 | 82 | 529 |

TABLE SB
Panel 1.3D

| | Rel. Exp. (%) Ag3205, | | Rel. Exp. (%) Ag3205, |
|---|---|---|---|
| Tissue Name | Run 165527062 | Tissue Name | Run 165527062 |
| Liver adenocarcinoma | 0.0 | Kidney (fetal) | 0.0 |
| Pancreas | 0.0 | Renal ca. 786-0 | 0.0 |
| Pancreatic ca. CAPAN2 | 6.0 | Renal ca. A498 | 0.0 |
| Adrenal gland | 4.4 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 5.4 |
| Brain (fetal) | 10.4 | Liver | 0.0 |
| Brain (whole) | 9.2 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 5.4 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 5.5 | Lung ca. (small cell) | 0.0 |
| | | NCI-H69 | |
| Cerebral Cortex | 3.5 | Lung ca. (s. cell var.) | 5.1 |
| | | SHP-77 | |
| Spinal cord | 0.0 | Lung ca. (large | 0.0 |
| | | cell) NCI-H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) SW | 5.3 |
| | | 900 | |
| astrocytoma SNB-75 | 0.0 | Lung ca. (squam.) | 4.7 |
| | | NCI-H596 | |
| glioma SNB-19 | 0.0 | Mammary gland | 7.3 |
| glioma U251 | 3.6 | Breast ca.* (pl. ef) | 0.0 |
| | | MCF-7 | |
| glioma SF-295 | 0.0 | Breast ca.* (pl. ef) | 0.0 |
| | | MDA-MB-231 | |
| Heart (fetal) | 0.0 | Breast ca.* (pl. ef) | 6.8 |
| | | T47D | |
| Heart | 2.9 | Breast ca. BT-549 | 4.9 |
| Skeletal muscle (fetal) | 0.0 | Breast ca. MDA-N | 0.0 |
| Skeletal muscle | 6.6 | Ovary | 0.0 |
| Bone marrow | 0.0 | Ovarian ca. OVCAR-3 | 0.0 |
| Thymus | 4.1 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca.* (ascites) | 0.0 |
| | | SK-OV-3 | |
| Small intestine | 0.0 | Uterus | 0.0 |
| Colon ca. SW480 | 0.0 | Placenta | 0.0 |
| Colon ca.* | 0.0 | Prostate | 0.0 |
| SW620 (SW480 met) | | | |
| Colon ca. HT29 | 0.0 | Prostate ca.* (bone | 0.0 |
| | | met) PC-3 | |
| Colon ca. HCT-116 | 0.0 | Testis | 100.0 |
| Colon ca. CaCo-2 | 0.0 | Melanoma Hs688(A).T | 3.3 |
| Colon ca. | 0.0 | Melanoma* (met) | 0.0 |
| tissue (ODO3866) | | Hs688(B).T | |
| Colon ca. HCC-2998 | 0.0 | Melanoma UACC-62 | 0.0 |
| Gastric ca.* (liver met) | 14.2 | Melanoma M14 | 0.0 |
| NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE SC
Panel 4D

| | Rel. Exp. (%) Ag3205, | | Rel. Exp. (%) Ag3205, |
|---|---|---|---|
| Tissue Name | Run 164531686 | Tissue Name | Run 164531686 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 1.1 |
| Secondary Th2 rest | 0.0 | HUVEC IL-11 | 0.5 |
| Secondary Tr1 rest | 0.0 | Lung Microvascular EC none | 1.5 |
| Primary Th1 act | 0.0 | Lung Microvascular EC | 0.0 |
| | | TNFalpha + IL-1beta | |
| Primary Th2 act | 0.0 | Microvascular Dermal EC | 2.5 |
| | | none | |
| Primary Tr1 act | 0.0 | Microsvasular Dermal EC | 1.9 |
| | | TNFalpha + IL-1beta | |
| Primary Th1 rest | 0.0 | Bronchial epithelium | 1.2 |
| | | TNFalpha + IL-1beta | |
| Primary Th2 rest | 0.0 | Small airway epithelium | 0.6 |
| | | none | |
| Primary Tr1 rest | 0.7 | Small airway epithelium | 1.1 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 0.0 |
| LAK cells IL-2 + IL-12 | 0.0 | Lupus kidney | 0.0 |
| LAK cells IL-2 + IFN | 0.0 | NCI-H292 none | 100.0 |
| gamma | | | |
| LAK cells IL-2 + IL-18 | 0.0 | NCI-H292 IL-4 | 1.4 |
| LAK cells | 0.0 | NCI-H292 IL-9 | 3.1 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNFalpha + IL-1beta | 0.0 |
| PBMC rest | 0.0 | Lung fibroblast none | 0.0 |
| PBMC PWM | 0.0 | Lung fibroblast | 0.0 |
| | | TNF alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 1.2 |
| Monocytes LPS | 0.0 | Colon | 0.0 |
| Macrophages rest | 0.0 | Lung | 0.9 |
| Macrophages LPS | 0.0 | Thymus | 0.0 |
| HUVEC none | 1.4 | Kidney | 1.1 |
| HUVEC starved | 0.0 | | |

Panel 1.3D Summary: Ag3205 Expression of the NOV31 gene, which is homologous to myelin P2, is restricted to a sample derived from the testis (CT=34.7). Thus, expression of this gene could be used to differentiate between this sample and other samples on this panel and as a marker of this tissue. Furthermore, the specific pattern of suggestion suggests that therapeutic modulation of this protein product may be useful in the treatment of male infertility or hypogonadism.

### REFERENCES

Schmitt M C, Jamison R S, Orgebin-Crist M C, Ong D E. A novel, testis-specific member of the cellular lipophilic transport protein superfamily, deduced from a complimentary deoxyribonucleic acid clone. Biol Reprod August 1994;51(2):239-45

A novel member of the cellular lipophilic transport protein superfamily was identified after an antiserum raised against cellular retinoic acid-binding protein (CRABP) was found also to contain antibodies against another 15-kDa protein present in the cytosol of pubertal and adult rat testis. These antibodies were used to screen a rat testis cDNA expression library and isolate a 561-bp clone containing a full open reading frame from which the sequence of a novel 132 amino acid protein was deduced. The protein has 58% amino acid sequence identity to bovine myelin P2, 58% identity to murine adipocyte lipid-binding protein, and 40% identity to rat CRABP. Although the endogenous ligand has not yet been identified, conservation of residues involved in the binding of carboxylate groups suggests that the ligand is a fatty acid or an acidic retinoid. Tissue-specific expression was examined by Northern analysis and immunolocalization and appears to be restricted to late germ cells within the testis and epididymis. Immunostaining was first detectable in mid-pachytene spermatocytes and increased in intensity as these cells progressed to elongated spermatids, suggesting that this testis lipid-binding protein has a specific role in sperm development.

PMID: 7948479

Panel 4D Summary: Ag3205 Expression of the CG57344-01 gene is restricted to a sample derived from untreated NCI-H292 cells (CT=31.9). Thus, expression of this gene could be used as a marker of this cell type.

T. NOV32—CG57346-01 and CG57346-02: Testis Lipid Binding Protein

Expression of the NOV32 gene was assessed using the primer-probe set Ag3206, described in Table TA. Results of the RTQ-PCR runs are shown in Tables TB and TC.

TABLE TA

Probe Name Ag3206

| | | | | |
|---|---|---|---|---|
| | | | | SEQ ID |
| Primers | Sequences | Length | Start Position | NO. |

| | | | | |
|---|---|---|---|---|
| Forward | 5′-agtgttgatgggaaaatgatga-3′ | 22 | 160 | 530 |
| Probe | TET-5′-ccataagaacagaaagttctttccaggaca-3′-TAMRA | 30 | 182 | 531 |
| Reverse | 5′-ccccagcttgaaggagatc-3′ | 19 | 216 | 532 |

TABLE TB
Panel 1.3D

| | Rel. Exp. (%) Ag3206, | | Rel. Exp. (%) Ag3206, |
|---|---|---|---|
| Tissue Name | Run 165527079 | Tissue Name | Run 165527079 |
| Liver adenocarcinoma | 0.0 | Kidney (fetal) | 10.5 |
| Pancreas | 0.0 | Renal ca. 786-0 | 0.0 |
| Pancreatic ca. CAPAN2 | 17.8 | Renal ca. A498 | 15.6 |
| Adrenal gland | 0.0 | Renal ca. RXF 393 | 0.0 |
| Thyroid | 0.0 | Renal ca. ACHN | 14.8 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 9.9 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) | 4.5 |
| | | LX-1 | |
| Brain (thalamus) | 0.0 | Lung ca (small cell) | 0.0 |
| | | NCI-H69 | |
| Cerebral Cortex | 0.0 | Lung ca. (s. cell var.) | 18.0 |
| | | SHP-77 | |
| Spinal cord | 33.4 | Lung ca. (large | 41.2 |
| | | cell) NCI-H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) SW | 0.0 |
| | | 900 | |
| astrocytoma SNB-75 | 11.7 | Lung ca. (squam.) | 0.0 |
| | | NCI-H596 | |
| glioma SNB-19 | 0.0 | Mammary gland | 14.4 |
| glioma U251 | 0.0 | Breast ca.* (pl. ef) | 0.0 |
| | | MCF-7 | |
| glioma SF-295 | 0.0 | Breast ca.* (pl. ef) | 0.0 |
| | | MDA-MB-231 | |
| Heart (fetal) | 0.0 | Breast ca.* (pl. ef) | 0.0 |
| | | T47D | |
| Heart | 15.5 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 14.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 11.6 |
| Stomach | 0.0 | Ovarian ca.* (ascites) | 0.0 |
| | | SK-OV-3 | |
| Small intestine | 0.0 | Uterus | 0.0 |
| Colon ca. SW480 | 0.0 | Placenta | 0.0 |
| Colon ca.* | 0.0 | Prostate | 0.0 |
| SW620 (SW480 met) | | | |
| Colon ca. HT29 | 0.0 | Prostate ca.* (bone | 100.0 |
| | | met) PC-3 | |
| Colon ca. HCT-116 | 0.0 | Testis | 27.5 |
| Colon ca. CaCo-2 | 42.0 | Melanoma Hs688(A).T | 0.0 |
| Colon ca. | 0.0 | Melanoma* (met) | 0.0 |
| tissue (ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX IMV1 | 0.0 |
| Trachea | 0.0 | Melanoma* (met) SK- | 0.0 |
| | | MEL-5 | |
| Kidney | 0.0 | Adipose | 0.0 |

TABLE TC
Panel 4D

| | Rel. Exp. (%) Ag3206, | | Rel. Exp. (%) Ag3206, |
|---|---|---|---|
| Tissue Name | Run 164531735 | Tissue Name | Run 164531735 |
| Secondary Th1 act | 0.0 | HUVEC IL-1beta | 0.0 |
| Secondary Th2 act | 11.9 | HUVEC IFN gamma | 0.0 |
| Secondary Tr1 act | 0.0 | HUVEC TNF alpha + IFN | 12.6 |
| | | gamma | |
| Secondary Th1 rest | 11.9 | HUVEC TNF alpha + IL-4 | 15.9 |
| Secondary Th2 rest | 0.0 | HUVEC IL-11 | 0.0 |
| Secondary Tr1 rest | 0.0 | Lung Microvascular EC none | 75.8 |
| Primary Th1 act | 0.0 | Lung Microvascular EC | 100.0 |
| | | TNFalpha + IL-1beta | |
| Primary Th2 act | 0.0 | Microvascular Dermal EC | 72.2 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL-1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 29.7 |
| LAK cells IL-2 + IL-12 | 0.0 | Lupus kidney | 0.0 |
| LAK cells IL-2 + IFN | 0.0 | NCI-H292 none | 97.3 |
| gamma | | | |
| LAK cells IL-2 + IL-18 | 0.0 | NCI-H292 IL-4 | 0.0 |
| LAK cells | 0.0 | NCI-H292 IL-9 | 43.8 |
| PMA/ionomycin | | | |
| NK Cells IL-2 rest | 7.8 | NCI-H292 IL-13 | 24.0 |
| Two Way MLR 3 day | 0.0 | NCI-H292 IFN gamma | 12.7 |
| Two Way MLR 5 day | 0.0 | HPAEC none | 14.3 |
| Two Way MLR 7 day | 0.0 | HPAEC TNF alpha + IL-1 beta | 33.2 |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 16.2 |
| Ramos (B cell) | 0.0 | Lung fibroblast IL-13 | 15.9 |
| ionomycin | | | |
| B lymphocytes PWM | 0.0 | Lung fibroblast IFN gamma | 0.0 |
| B lymphocytes CD40L | 0.0 | Dermal fibroblast CCD1070 | 0.0 |
| and IL-4 | | rest | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 | 0.0 |
| PMA/ionomycin | | IL-1 beta | |
| Dendritic cells none | 0.0 | Dermal fibroblast IFN | 0.0 |
| | | gamma | |
| Dendritic cells LPS | 0.0 | Dermal fibroblast IL-4 | 15.0 |
| Dendritic cells anti- | 0.0 | IBD Colitis 2 | 27.4 |
| CD40 | | | |
| Monocytes rest | 0.0 | IBD Crohn's | 0.0 |
| Monocytes LPS | 0.0 | Colon | 6.7 |
| Macrophages rest | 0.0 | Lung | 0.0 |
| Macrophages LPS | 0.0 | Thymus | 0.0 |
| HUVEC none | 27.7 | Kidney | 0.0 |
| HUVEC starved | 20.0 | | |

Panel 1.3D Summary: Ag3206 Expression of the NOV32 gene is restricted to a sample derived from a prostate cancer cell line (CT=34.9). Thus, expression of this gene could be used to differentiate between this sample and other samples on this panel and as a marker to detect the presence of prostate cancer. Furthermore, therapeutic modulation of the expression or function of this gene may be effective in the treatment of prostate cancer.

Panel 4D Summary: Ag3206 Expression of the NOV32 gene is primarily restricted to a cluster of samples derived from microvasculature of the lung and the dermis suggesting a role for this gene in the maintenance of the integrity of the microvasculature. Therefore, therapeutics designed for this putative protein could be beneficial for the treatment of diseases associated with damaged microvasculature including heart diseases or inflammatory diseases, such as psoriasis, asthma, and chronic obstructive pulmonary diseases.

U. NOV33—CG57356-01: Novel Intracellular Thrombospondin Domain Containing Protein

Expression of the NOV33 gene was assessed using the primer-probe set Ag672, described in Table UA. Results of the RTQ-PCR runs are shown in Table UB.

TABLE UA

Probe Name Ag672

| | | | | |
|---|---|---|---|---|
| | | | | SEQ ID |
| Primers | Sequences | Length | Start Position | NO: |

| | | | | |
|---|---|---|---|---|
| Forward | 5′-ccagatcctttctccttgatct-3′ | 22 | 1076 | 533 |
| Probe | TET-5′-ccaaactttccagatctttccaaagctg-3′-TAMRA | 28 | 1047 | 534 |
| Reverse | 5′-tgacctggatatttggattctg-3′ | 22 | 1014 | 535 |

TABLE UB
Panel 1.1

| | Rel. Exp. (%) Ag672, | | Rel. Exp. (%) Ag672, |
|---|---|---|---|
| Tissue Name | Run 109210121 | Tissue Name | Run 109210121 |
| Adrenal gland | 3.8 | Renal ca. UO-31 | 0.1 |
| Bladder | 24.3 | Renal ca. RXF 393 | 11.6 |
| Brain (amygdala) | 0.2 | Liver | 5.0 |
| Brain (cerebellum) | 3.7 | Liver (fetal) | 0.0 |
| Brain (hippocampus) | 3.1 | Liver ca. (hepatoblast) | 0.6 |
| | | HepG2 | |
| Brain (substantia nigra) | 14.4 | Lung | 6.5 |
| Brain (thalamus) | 10.4 | Lung (fetal) | 57.4 |
| Cerebral Cortex | 3.2 | Lung ca. (non-s. cell) | 0.6 |
| | | HOP-62 | |
| Brain (fetal) | 1.3 | Lung ca. (large | 4.9 |
| | | cell) NCI-H460 | |
| Brain (whole) | 1.5 | Lung ca. (non-s. cell) | 0.1 |
| | | NCI-H23 | |
| glio/astro U-118-MG | 15.3 | Lung ca. (non-s. cl) | 0.0 |
| | | NCI-H522 | |
| astrocytoma SF-539 | 0.0 | Lung ca. (non-sm. cell) | 2.5 |
| | | A549 | |
| astrocytoma SNB-75 | 0.6 | Lung ca. (s. cell var.) | 0.1 |
| | | SHP-77 | |
| astrocytoma SW1783 | 0.0 | Lung ca. (small cell) | 11.6 |
| | | LX-1 | |
| glioma U251 | 1.0 | Lung ca. (small cell) | 17.7 |
| | | NCI-H69 | |
| glioma SF-295 | 0.0 | Lung ca. (squam.) SW | 29.3 |
| | | 900 | |
| glioma SNB-19 | 0.8 | Lung ca. (squam.) | 63.3 |
| | | NCI-H596 | |
| glio/astro U87-MG | 3.7 | Lymph node | 3.4 |
| neuro*; met SK-N-AS | 0.0 | Spleen | 3.8 |
| Mammary gland | 43.5 | Thymus | 2.5 |
| Breast ca. BT-549 | 0.8 | Ovary | 0.8 |
| Breast ca. MDA-N | 0.1 | Ovarian ca. IGROV-1 | 5.0 |
| Breast ca.* (pl. ef) T47D | 9.6 | Ovarian ca. OVCAR-3 | 6.3 |
| Breast ca.* (pl. ef) MCF- | 2.7 | Ovarian ca. OVCAR-4 | 0.0 |
| 7 | | | |
| Breast ca.* (pl. ef) MDA- | 1.2 | Ovarian ca. OVCAR-5 | 6.3 |
| MB-231 | | | |
| Small intestine | 6.2 | Ovarian ca. OVCAR-8 | 1.8 |
| Colorectal | 0.1 | Ovarian ca.* (ascites) | 2.7 |
| | | SK-OV-3 | |
| Colon ca. HT29 | 0.0 | Pancreas | 54.0 |
| Colon ca. CaCo-2 | 2.1 | Pancreatic ca. CAPAN2 | 0.1 |
| Colon ca. HCT-15 | 1.7 | Pituitary gland | 14.0 |
| Colon ca. HCT-116 | 1.7 | Placenta | 100.0 |
| Colon ca. HCC-2998 | 2.2 | Prostate | 1.0 |
| Colon ca. SW480 | 19.1 | Prostate ca.* (bone | 0.0 |
| | | met) PC-3 | |
| Colon ca.* SW620 | 0.8 | Salivary gland | 42.3 |
| (SW480 met) | | | |
| Stomach | 6.7 | Trachea | 23.8 |
| Gastric ca. (liver met) | 9.5 | Spinal cord | 6.7 |
| NCI-N87 | | | |
| Heart | 23.0 | Testis | 0.0 |
| Skeletal muscle (Fetal) | 1.6 | Thyroid | 78.5 |
| Skeletal muscle | 13.1 | Uterus | 1.6 |
| Endothelial cells | 0.2 | Melanoma M14 | 0.0 |
| Heart (Fetal) | 4.2 | Melanoma LOX IMVI | 0.0 |
| Kidney | 6.4 | Melanoma UACC-62 | 0.0 |
| Kidney (fetal) | 5.0 | Melanoma SK-MEL- | 65.5 |
| | | 28 | |
| Renal ca. 786-0 | 2.0 | Melanoma* (met) SK- | 37.1 |
| | | MEL-5 | |
| Renal ca. A498 | 13.2 | Melanoma Hs688(A).T | 0.0 |
| Renal ca. ACHN | 0.1 | Melanoma* (met) | 0.0 |
| | | Hs688(B).T | |
| Renal ca. TK-10 | 36.1 | | |

Panel 1.1 Summary: Ag672 The results obtained in this experiment are comparable to what is observed in Panel 1. Expression of the NOV33 gene is primarily associated with normal tissues on this panel. Highest expression is seen in placenta (CT=25), thyroid (CT=25.2), pancreas (CT=25.7), and mammary gland (CT=26). Therefore, the NOV33 gene might be useful as a marker to distinguish these tissues. In addition, the observed expression in mammary gland and placenta suggests a potential role for the NOV33 gene product in pregnancy. Interestingly, expression of this gene is much lower in 5/5 breast cancer cell lines when compared to normal breast. This suggests that replacement of the NOV33 gene product using protein therapeutics, peptides or gene therapy would be valuable in the treatment of breast cancer.

In addition, the NOV33 gene is expressed throughout the CNS with low to moderate expression detected in amygdala, cerebellum, hippocampus, substantia nigra, thalamus and cerebral cortex. Expression of this gene is decreased in CNS cancer cell lines relative to normal brain tissues. The secreted protein encoded for by the NOV33 gene contains homology to thrombospondin, suggesting it may play a role in inhibiting angiogenesis. Therefore, treatment with the NOV33 protein, or in vivo modulation of the gene or the protein product may therefore be of use in slowing the growth/inhibiting CNS tumors. Selective removal of this protein via synthetic antibodies may help to increase vascularization in CNS tissue undergoing repair/regeneration.

Among the metabolically relevant tissues, the NOV33 gene is expressed at high levels in thyroid and pancreas and at more moderate levels in adrenal gland, pituitary gland, heart, and skeletal muscle. Therefore, this gene product may have utility as a drug treatment for any or all diseases of the thyroid gland and pancreas as well as other metabolic and neuroendocrine diseases. Interestingly, this gene is more highly expressed in adult liver (CT=29) than in fetal liver (CT=40), suggesting that the NOV33 gene would be a useful marker for differentiating between the adult and fetal liver.

V. NOV34a —CG57258-01: Ornithine Decarboxylase

Expression of the NOV34a gene was assessed using the primer-probe set Ag3148, described in Table VA. Results of the RTQ-PCR runs are shown in Tables VB, VC, VD and VE.

TABLE VA

Probe Name Ag3148

| | | | | |
|---|---|---|---|---|
| Primers | Sequences | Length | Start Position | SEQ ID NO: |

| | | | | |
|---|---|---|---|---|
| Forward | 5′-acctgctgaaggaactcactct-3′ | 22 | 112 | 536 |
| Probe | TET-5′-ctcacaggacgaggtagctgccttct-3′-TAMRA | 26 | 140 | 537 |
| Reverse | 5′-gcaaaagtgcttcctcactatg-3′ | 22 | 185 | 538 |

TABLE VB
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3148, Run | Ag3148, Run | | Ag3148, Run | Ag3148, Run |
| Tissue Name | 209057912 | 249265913 | Tissue Name | 209057912 | 249265913 |
| AD 1 Hippo | 14.6 | 13.9 | Control (Path) | 4.9 | 3.7 |
| | | | 3 Temporal | | |
| | | | Ctx | | |
| AD 2 Hippo | 37.6 | 27.7 | Control (Path) | 22.2 | 26.1 |
| | | | 4 Temporal | | |
| | | | Ctx | | |
| AD 3 Hippo | 8.2 | 7.3 | AD 1 | 8.8 | 8.6 |
| | | | Occipital Ctx | | |
| AD 4 Hippo | 12.8 | 12.9 | AD 2 | 0.0 | 0.0 |
| | | | Occipital Ctx | | |
| | | | (Missing) | | |
| AD 5 Hippo | 67.4 | 49.7 | AD 3 | 5.6 | 1.7 |
| | | | Occipital Ctx | | |
| AD 6 Hippo | 29.1 | 29.7 | AD 4 | 21.8 | 21.2 |
| | | | Occipital Ctx | | |
| Control 2 | 51.8 | 50.0 | AD 5 | 71.2 | 9.2 |
| Hippo | | | Occipital Ctx | | |
| Control 4 | 14.9 | 17.0 | AD 6 | 9.5 | 59.0 |
| Hippo | | | Occipital Ctx | | |
| Control (Path) | 4.1 | 3.7 | Control 1 | 6.9 | 3.2 |
| 3 Hippo | | | Occipital Ctx | | |
| AD 1 | 1.8 | 13.9 | Control 2 | 77.9 | 77.4 |
| Temporal Ctx | | | Occipital Ctx | | |
| AD 2 | 35.6 | 38.2 | Control 3 | 17.1 | 14.2 |
| Temporal Ctx | | | Occipital Ctx | | |
| AD 3 | 2.0 | 5.5 | Control 4 | 9.2 | 8.8 |
| Temporal Ctx | | | Occipital Ctx | | |
| AD 4 | 20.3 | 14.9 | Control (Path) | 100.0 | 78.5 |
| Temporal Ctx | | | 1 Occipital | | |
| | | | Ctx | | |
| AD 5 Inf | 79.6 | 70.2 | Control (Path) | 9.3 | 8.1 |
| Temporal Ctx | | | 2 Occipital | | |
| | | | Ctx | | |
| AD 5 Sup | 35.8 | 30.8 | Control (Path) | 2.3 | 1.1 |
| Temporal Ctx | | | 3 Occipital | | |
| | | | Ctx | | |
| AD 6 Inf | 19.2 | 16.5 | Control (Path) | 10.1 | 12.8 |
| Temporal Ctx | | | 4 Occipital | | |
| | | | Ctx | | |
| AD 6 Sup | 15.7 | 28.5 | Control 1 | 11.3 | 12.0 |
| Temporal Ctx | | | Parietal Ctx | | |
| Control 1 | 6.7 | 7.7 | Control 2 | 32.8 | 37.1 |
| Temporal Ctx | | | Parietal Ctx | | |
| Control 2 | 58.6 | 67.8 | Control 3 | 19.1 | 16.6 |
| Temporal Ctx | | | Parietal Ctx | | |
| Control 3 | 16.3 | 17.9 | Control (Path) | 87.1 | 100.0 |
| Temporal Ctx | | | 1 Parietal Ctx | | |
| Control 3 | 10.4 | 9.5 | Control (Path) | 18.3 | 18.7 |
| Temporal Ctx | | | 2 Parietal Ctx | | |
| Control (Path) | 72.7 | 69.7 | Control (Path) | 4.4 | 2.0 |
| 1 Temporal | | | 3 Parietal Ctx | | |
| Ctx | | | | | |
| Control (Path) | 31.0 | 31.0 | Control (Path) | 26.6 | 3.4 |
| 2 Temporal | | | 4 Parietal Ctx | | |
| Ctx | | | | | |

TABLE VC
Panel 1.3D

| | Rel. Exp. (%) Ag3148, | | Rel. Exp. (%) Ag3148, |
|---|---|---|---|
| Tissue Name | Run 167994492 | Tissue Name | Run 167994492 |
| Liver adenocarcinoma | 27.4 | Kidney (fetal) | 15.4 |
| Pancreas | 6.5 | Renal ca. 786-0 | 5.9 |
| Pancreatic ca. CAPAN2 | 2.9 | Renal ca. A498 | 3.5 |
| Adrenal gland | 2.9 | Renal ca. RXF 393 | 13.6 |
| Thyroid | 8.2 | Renal ca. ACHN | 10.9 |
| Salivary gland | 2.1 | Renal ca. UO-31 | 0.0 |
| Pituitary gland | 7.6 | Renal ca. TK-10 | 3.7 |
| Brain (fetal) | 49.3 | Liver | 0.0 |
| Brain (whole) | 51.8 | Liver (fetal) | 0.0 |
| Brain (amygdala) | 32.8 | Liver ca. (hepatoblast) | 3.2 |
| | | HepG2 | |
| Brain (cerebellum) | 39.0 | Lung | 2.7 |
| Brain (hippocampus) | 40.9 | Lung (fetal) | 13.8 |
| Brain (substantia nigra) | 68.3 | Lung ca. (small cell) | 2.3 |
| | | LX-1 | |
| Brain (thalamus) | 49.3 | Lung ca. (small cell) | 1.1 |
| | | NCI-H69 | |
| Cerebral Cortex | 100.0 | Lung ca. (s. cell var.) | 5.6 |
| | | SHP-77 | |
| Spinal cord | 41.8 | Lung ca. (large | 1.3 |
| | | cell) NCI-H460 | |
| glio/astro U87-MG | 9.3 | Lung ca (non-sm. cell) | 7.4 |
| | | A549 | |
| glio/astro U-118-MG | 0.0 | Lung ca. (non-s. cell) | 1.2 |
| | | NCI-H23 | |
| astrocytoma SW1783 | 2.6 | Lung ca. (non-s. cell) | 21.9 |
| | | HOP-62 | |
| neuro*; met SK-N-AS | 1.4 | Lung ca (non-s. cl) | 7.2 |
| | | NCI-H522 | |
| astrocytoma SF-539 | 2.8 | Lung ca (squam.) SW | 3.8 |
| | | 900 | |
| astrocytoma SNB-75 | 6.6 | Lung ca. (squam.) | 0.0 |
| | | NCI-H596 | |
| glioma SNB-19 | 15.8 | Mammary gland | 13.8 |
| glioma U251 | 57.8 | Breast ca.* (pl. ef) | 0.8 |
| | | MCF-7 | |
| glioma SF-295 | 22.4 | Breast ca.* (pl. ef) | 12.7 |
| | | MDA-MB-231 | |
| Heart (fetal) | 36.9 | Breast ca.* (pl. ef) | 13.8 |
| | | T47D | |
| Heart | 4.1 | Breast ca. BT-549 | 1.4 |
| Skeletal muscle (fetal) | 13.4 | Breast ca. MDA-N | 1.8 |
| Skeletal muscle | 6.0 | Ovary | 22.2 |
| Bone marrow | 0.0 | Ovarian ca. OVCAR-3 | 0.6 |
| Thymus | 3.8 | Ovarian ca. OVCAR-4 | 10.0 |
| Spleen | 0.5 | Ovarian ca. OVCAR-5 | 19.5 |
| Lymph node | 3.7 | Ovarian ca. OVCAR-8 | 3.6 |
| Colorectal | 3.5 | Ovarian ca. IGROV-1 | 3.2 |
| Stomach | 2.0 | Ovarian ca.* (ascites) | 14.3 |
| | | SK-OV-3 | |
| Small intestine | 7.5 | Uterus | 7.5 |
| Colon ca. SW480 | 3.3 | Placenta | 0.0 |
| Colon ca.* | 6.8 | Prostate | 4.4 |
| SW620 (SW480 met) | | | |
| Colon ca. HT29 | 0.0 | Prostate ca.* (bone | 3.3 |
| | | met) PC-3 | |
| Colon ca. HCT-116 | 3.3 | Testis | 61.6 |
| Colon ca. CaCo-2 | 1.7 | Melanoma Hs688(A).T | 1.1 |
| Colon ca. | 3.2 | Melanoma* (met) | 2.0 |
| tissue (ODO3866) | | Hs688(B).T | |
| Colon ca. HCC-2998 | 1.2 | Melanoma UACC-62 | 14.9 |
| Gastric ca.* (liver met) | 9.6 | Melanoma M14 | 2.6 |
| NCI-N87 | | | |
| Bladder | 1.6 | Melanoma LOX IMVI | 4.7 |
| Trachea | 2.0 | Melanoma* (met) SK- | 7.3 |
| | | MEL-5 | |
| Kidney | 13.2 | Adipose | 2.9 |

TABLE VD
Panel 4D

| | Rel. Exp. (%) Ag3148, | | Rel. Exp. (%) Ag3148, |
|---|---|---|---|
| Tissue Name | Run 164528041 | Tissue Name | Run 164528041 |
| Secondary Th1 act | 25.9 | HUVEC IL-1beta | 0.0 |
| Secondary Th2 act | 18.6 | HUVEC IFN gamma | 2.6 |
| Secondary Tr1 act | 8.4 | HUVEC TNF alpha + IFN | 8.3 |
| | | gamma | |
| Secondary Th1 rest | 14.1 | HUVEC TNF alpha + IL4 | 6.8 |
| Secondary Th2 rest | 12.1 | HUVEC IL-11 | 3.9 |
| Secondary Tr1 rest | 24.7 | Lung Microvascular EC none | 10.4 |
| Primary Th1 act | 16.5 | Lung Microvascular EC | 12.8 |
| | | TNFalpha + IL-1beta | |
| Primary Th2 act | 16.0 | Microvascular Dermal EC | 7.5 |
| | | none | |
| Primary Tr1 act | 4.1 | Microsvasular Dermal EC | 13.9 |
| | | TNFalpha + IL-1beta | |
| Primary Th1 rest | 41.2 | Bronchial epithelium | 57.8 |
| | | TNFalpha + IL-1beta | |
| Primary Th2 rest | 53.2 | Small airway epithelium | 7.9 |
| | | none | |
| Primary Tr1 rest | 88.3 | Small airway epithelium | 45.1 |
| | | TNFalpha + IL-1beta | |
| CD45RA CD4 | 21.6 | Coronery artery SMC rest | 25.2 |
| lymphocyte act | | | |
| CD45RO CD4 | 10.6 | Coronery artery SMC | 18.2 |
| lymphocyte act | | TNFalpha + IL-1beta | |
| CD8 lymphocyte act | 2.8 | Astrocytes rest | 8.0 |
| Secondary CD8 | 7.4 | Astrocytes TNFalpha + IL- | 12.2 |
| lymphocyte rest | | 1beta | |
| Secondary CD8 | 12.4 | KU-812 (Basophil) rest | 4.4 |
| lymphocyte act | | | |
| CD4 lymphocyte none | 11.0 | KU-812 (Basophil) | 0.0 |
| | | PMA/ionomycin | |
| 2ry Th1/Th2/Tr1_anti- | 10.1 | CCD1106 (Keratinocytes) | 5.7 |
| CD95 CH11 | | none | |
| LAK cells rest | 33.9 | CCD1106 (Keratinocytes) | 31.9 |
| | | TNFalpha + IL-1beta | |
| LAK cells IL-2 | 11.7 | Liver cirrhosis | 5.9 |
| LAK cells IL-2 + IL-12 | 8.2 | Lupus kidney | 11.7 |
| LAK cells IL-2 + IFN | 30.8 | NCI-H292 none | 87.1 |
| gamma | | | |
| LAK cells IL-2 + IL-18 | 30.8 | NCI-H292 IL-4 | 100.0 |
| LAK cells | 5.8 | NCI-H292 IL-9 | 48.3 |
| PMA/ionomycin | | | |
| NK Cells IL-2 rest | 26.4 | NCI-H292 IL-13 | 47.3 |
| Two Way MLR 3 day | 16.6 | NCI-H292 IFN gamma | 49.3 |
| Two Way MLR 5 day | 0.0 | HPAEC none | 2.8 |
| Two Way MLR 7 day | 8.6 | HPAEC TNF alpha + IL-1 | 5.7 |
| | | beta | |
| PBMC rest | 15.5 | Lung fibroblast none | 21.6 |
| PBMC PWM | 25.0 | Lung fibroblast TNF alpha + | 41.5 |
| | | IL-1 beta | |
| PBMC PHA-L | 9.5 | Lung fibroblast IL-4 | 7.2 |
| Ramos (B cell) none | 0.0 | Lung fibroblast IL-9 | 2.9 |
| Ramos (B cell) | 0.0 | Lung fibroblast IL-13 | 4.3 |
| ionomycin | | | |
| B lymphocytes PWM | 18.7 | Lung fibroblast IFN gamma | 15.2 |
| B lymphocytes CD40L | 19.9 | Dermal fibroblast CCD1070 | 14.9 |
| and IL-4 | | rest | |
| EOL-1 dbcAMP | 32.5 | Dermal fibroblast CCD1070 | 82.9 |
| | | TNF alpha | |
| EOL-1 dbcAMP | 16.5 | Dermal fibroblast CCD1070 | 10.1 |
| PMA/ionomycin | | IL-1 beta | |
| Dendritic cells none | 2.5 | Dermal fibroblast IFN | 21.8 |
| | | gamma | |
| Dendritic cells LPS | 44.4 | Dermal fibroblast IL-4 | 0.7 |
| Dendritic cells anti- | 0.0 | IBD Colitis 2 | 2.3 |
| CD40 | | | |
| Monocytes rest | 0.0 | IBD Crohn's | 7.3 |
| Monocytes LPS | 5.9 | Colon | 4.2 |
| Macrophages rest | 3.3 | Lung | 13.9 |
| Macrophages LPS | 42.3 | Thymus | 55.5 |
| HUVEC none | 0.0 | Kidney | 53.6 |
| HUVEC starved | 4.2 | | |

TABLE VE
Panel CNS 1

| | Rel. Exp. (%) Ag3148, | | Rel. Exp. (%) Ag3148, |
|---|---|---|---|
| Tissue Name | Run 171694168 | Tissue Name | Run 171694168 |
| BA4 Control | 39.5 | BA17 PSP | 7.7 |
| BA4 Control2 | 55.1 | BA17 PSP2 | 5.3 |
| BA4 | 7.9 | Sub Nigra Control | 29.5 |
| Alzheimer's2 | | | |
| BA4 Parkinson's | 36.9 | Sub Nigra Control2 | 35.4 |
| BA4 Parkinson's2 | 55.5 | Sub Nigra | 24.1 |
| | | Alzheimer's2 | |
| BA4 Huntington's | 43.2 | Sub Nigra | 70.7 |
| | | Parkinson's2 | |
| BA4 | 4.8 | Sub Nigra | 75.8 |
| Huntington's2 | | Huntington's | |
| BA4 PSP | 9.3 | Sub Nigra | 33.9 |
| | | Huntington's2 | |
| BA4 PSP2 | 19.9 | Sub Nigra PSP2 | 9.2 |
| BA4 Depression | 19.2 | Sub Nigra Depression | 7.7 |
| BA4 Depression2 | 7.5 | Sub Nigra | 1.2 |
| | | Depression2 | |
| BA7 Control | 31.0 | Glob Palladus Control | 19.2 |
| BA7 Control2 | 47.0 | Glob Palladus | 13.7 |
| | | Control2 | |
| BA7 | 3.2 | Glob Palladus | 37.6 |
| Alzheimer's2 | | Alzheimer's | |
| BA7 Parkinson's | 15.8 | Glob Palladus | 6.6 |
| | | Alzheimer's2 | |
| BA7 Parkinson's2 | 41.2 | Glob Palladus | 45.4 |
| | | Parkinson's | |
| BA7 Huntington's | 36.3 | Glob Palladus | 17.9 |
| | | Parkinson's2 | |
| BA7 | 13.5 | Glob Palladus PSP | 5.7 |
| Huntington's2 | | | |
| BA7 PSP | 18.6 | Glob Palladus PSP2 | 15.3 |
| BA7 PSP2 | 29.7 | Glob Palladus | 6.5 |
| | | Depression | |
| BA7 Depression | 5.3 | Temp Pole Control | 4.6 |
| BA9 Control | 27.4 | Temp Pole Control2 | 42.6 |
| BA9 Control2 | 87.1 | Temp Pole | 2.5 |
| | | Alzheimer's | |
| BA9 Alzheimer's | 3.6 | Temp Pole | 0.0 |
| | | Alzheimer's2 | |
| BA9 | 18.9 | Temp Pole Parkinson's | 26.1 |
| Alzheimer's2 | | | |
| BA9 Parkinson's | 22.1 | Temp Pole | 21.8 |
| | | Parkinson's2 | |
| BA9 Parkinson's2 | 54.0 | Temp Pole | 40.1 |
| | | Huntington's | |
| BA9 Huntington's | 54.3 | Temp Pole PSP | 2.5 |
| BA9 | 25.7 | Temp Pole PSP2 | 1.4 |
| Huntington's2 | | | |
| BA9 PSP | 10.4 | Temp Pole | 4.3 |
| | | Depression2 | |
| BAP PSP2 | 4.8 | Cing Gyr Control | 82.9 |
| BA9 Depression | 14.7 | Cing Gyr Control | 34.4 |
| BA9 Depression2 | 5.7 | Cing Gyr Alzheimer's | 33.7 |
| BA17 Control | 40.1 | Cing Gyr | 8.9 |
| | | Alzheimer's2 | |
| BA17 Control2 | 57.8 | Cing Gyr Parkinson's | 41.2 |
| BA17 | 4.1 | Cing Gyr Parkinson's2 | 44.8 |
| Alzheimer's2 | | | |
| BA17 Parkinson's | 38.2 | Cing Gyr Huntington's | 100.0 |
| BA17 | 31.0 | Cing Gyr | 22.4 |
| Parkinson's2 | | Huntington's2 | |
| BA17 | 30.8 | Cing Gyr PSP | 6.8 |
| Huntington's | | | |
| BA17 | 5.2 | Cing Gyr PSP2 | 6.1 |
| Huntington's2 | | | |
| BA17 Depression | 7.8 | Cing Gyr Depression | 2.1 |
| BA17 | 20.2 | Cing Gyr Depression2 | 4.4 |
| Depression2 | | | |

CNS_neurodegeneration_v1.0 Summary: Ag3148 The NOV34a gene is found to be down-regulated approximately 2-fold in the temporal cortex of Alzheimer's disease patients when compared to normal controls (p 0.015 analysis by ANCOVA). Multiple research groups have shown ornithine decarboxylase to be upregulated in the AD brain; the downregulation of this form suggests a shift between polyamine biosynthesis pathways during neurodegeneration. The polyamine system has also been implicated in seizure, stroke, depression and schizophrenia; therefore this gene is an excellent drug target for any of the above disorders.

### REFERENCES

Bernstein H G, Muller M. The cellular localization of the L-ornithine decarboxylase/polyamine system in normal and diseased central nervous systems. Prog Neurobiol April 1999;57(5):485-505

Natural polyamines, spermidine and spermine, and their precursor putrescine, are of considerable importance for the developing and mature nervous system. They exhibit a number of neurophysiological and metabolic effects in the nervous system, including control of nucleic acid and protein synthesis, modulation of ionic channels and calcium-dependent transmitter release. The polyamine system is also known to be involved in various brain pathologic events (seizures, stroke, Alzheimer's disease and others). While cerebral polyamine concentrations and the activities of polyamine-metabolizing enzymes have been studied in great detail, much less is known about the cells that are responsible for cerebral polyamine synthesis and interconversion. With the present review the attempt is made to show how exact knowledge about the regional distribution and cellular localization of polyamines and the polyamine-synthesizing enzymatic machinery (and especially of L-ornithine decarboxylase) may help to better understand the functional interplay between polyamines and other endogenous agents (transmitters, receptors, growth factors neuroactive drugs etc.). Polyamines have been localized both in neurones and glial cells. However, the main cellular locus of the ODC is the neuron-both in the immature and adult central nervous system. Each period of normal brain development and ageing seems to have its own, characteristic temporo-spatial pattern of neuronal ODC expression During strong functional activation (kindling, epileptic seizures, neural transplantation) astrocytes and other non-neuronal cells do also express ODC and other polyamine-metabolizing enzymes. Astroglial expression of ODC is accompanied by an increase in glial fibrillary acidic protein in these cells. This shift in the cellular mechanisms of polyamine metabolism is currently far from being understood. In human brain diseases (Alzheimer's disease, schizophrenia) certain neurones show an increased expression of ODC, the first and rate-limiting enzyme of polyamine metabolism. Since polyamines are structurally related to psychoactive drugs (neuroleptics, antidepressants) the polyamine system might be of importance as a putative target for drug intervention in psychiatry.

Morrison L D, Cao X C, Kish S J. Ornithine decarboxylase in human brain: influence of aging, regional distribution, and Alzheimer's disease. J Neurochem July 1998;71(1):288-94

Although experimental animal data have implicated omithine decarboxylase, a key regulatory enzyme of polyamine biosynthesis, in brain development and function, little information is available on this enzyme in normal or abnormal human brain. We examined the influence, in autopsied human brain, of postnatal development and aging, regional distribution, and Alzheimer's disease on the activity of ornithine decarboxylase. Consistent with animal data, human brain ornithine decarboxylase activity was highest in the perinatal period, declining sharply (by approximately 60%) during the first year of life to values that remained generally unchanged up to senescence. In adult brain, a moderately heterogeneous regional distribution of enzyme activity was observed, with high levels in the thalamus and occipital cortex and low levels in cerebellar cortex and putamen. In the Alzheimer's disease group, mean ornithine decarboxylase activity was significantly increased in the temporal cortex (+76%), reduced in occipital cortex (−70%), and unchanged in hippocampus and putamen. In contrast, brain enzyme activity was normal in patients with the neurodegenerative disorder spinocerebellar ataxia type I. Our demonstration of ornithine decarboxylase activity in neonatal and adult human brain suggests roles for ornithine decarboxylase in both developing and mature brain function, and we provide further evidence for the involvement of abnormal polyamine system activity in Alzheimer's disease.

Panel 1.3D Summary: Ag3148 Highly brain-preferential expression of the NOV34a gene indicates a specific role for this gene in the CNS. Polyamine synthesis by ornithine decarboxylase is thought to play a neuroprotective role or recovery role, or both, after transient focal ischemia in the CNS. Therefore, agents that enhance the activity of this gene product are likely to have medical utility as therapeutics for the treatment of stroke and trauma. Other diseases that involve oxidative damage, such as neurodegenerative diseases like Alzheimer's disease, also involve defensive mechanisms in which ornithine decarboxylase plays a role. Therefore, agents that enhance the activity of this gene are likely to have medical utility as therapeutics for the treatment of neurodegenerative diseases such as Alzheimer's disease.

In addition, significant levels of expression are seen in brain and liver cancer cell lines. Thus, expression of this gene could be used to differentiate between these samples and other samples on this panel and as a marker to detect the presence of these cancer. Furthermore, therapeutic modulation of the expression or function of this gene may be effective in the treatment of brain and liver cancer.

### REFERENCES

Yatin S M, Yatin M, Aulick T, Ain K B, Butterfield D A. Alzheimeres amyloid beta-peptide associated free radicals increase rat embryonic neuronal polyamine uptake and omithine decarboxylase activity: protective effect of vitamin E. Neurosci Lett Mar. 19, 1999;263(1):17-20

Recent evidence indicates that alterations in brain polyamine metabolism may be critical for nerve cell survival after a free radical initiated neurodegenerative process. It has been shown previously that A beta(1-42) and A beta(25-35) are toxic to neurons through a free radical dependent oxidative mechanism. Treatment of rat embryonic hippocampal neuronal cultures with A beta-peptides increased ornithine decarboxylase (ODC) activity and spermidine uptake, suggesting that oxidative stress upregulates the polyamine mechanism for the repair of free radical damage. Pretreatment of the cells with vitamin E prior to A beta exposure decreased ODC activity and spernidine uptake to control level. This study is the first to demonstrate that A beta treated cells show an increased polyamine metabolism in response to free radical mediated oxidative stress and that the free radical scavenger vitamin E prevents these attenuations. These results are discussed with reference to Alzbeimer's disease.

Kaasinen K, Koistinaho J, Alhonen L, Janne J. Overexpression of spermidineispermine N-acetyltransferase in transgenic mice protects the animals from kainate-induced toxicity. Eur J Neurosci February 2000; 12(2):540-8

We recently generated a transgenic mouse line with activated polyamine catabolism through overexpression of spermidine/spermine N1-acetyltransferase (SSAT). A detailed analysis of brain polyamine concentrations indicated that all brain regions of these animals showed distinct signs of activated polyamine catabolism, e.g. overaccumulation of putrescine (three- to 17-fold), appearance of N1-acetylspermidine and decreases in spermidine concentrations. In situ hybridization analyses revealed a marked overexpression of SSAT-specific mRNA all over the brain tissue of the transgenic animals. The transgenic animals appeared to tolerate subcutaneous injections of high-dose kainate substantially better as their overall mortality was less than 50% of that of their syngenic littermates. We used the expression of glial fibrillary acidic protein (GFAP) as a marker of brain injury in response to kainate. In situ hybridization analysis with GFAP oligonucleotide up to 7 days after the administration of sublethal kainate doses showed reduced GFAP expression in transgenic animals in comparison with their non-transgenic littermates. This difference was especially striking in the cerebral cortex of the transgenic mice where the exposure to kainate hardly induced GFAP expression. The treatment with kainate likewise resulted in loss of the hippocampal (CA3) neurons in non-transgenic but not transgenic animals. These results support our earlier findings indicating that elevated concentrations of brain putrescine, irrespective whether derived from an overexpression of ornithine decarboxylase, or as shown here, from an overexpression of SSAT, play in all likelihood a neuroprotective role in brain injury.

Kilpelainen P, Rybnikova E, Hietala O, Pelto-Huikko M. Expression of ODC and its regulatory protein antizyme in the adult rat brain. J Neurosci Res Dec. 1, 2000;62(5):675-85

Ornithine decarboxylase and its inhibitor protein, antizyme are key regulators of polyamine biosynthesis. We examined their expression in the adult rat brain using in situ hybridization and immunocytochemistry. Both genes were widely expressed and their expression patterns were mostly overlapping and relatively similar. The levels of antizyme mRNA were always higher than those of ornithine decarboxylase mRNA. The highest expression for both genes was detected in the cerebellar cortex, hippocampus, hypothalamic paraventricular and supraoptic nuclei, locus coeruleus, olfactory bulb, piriform cortex and pontine nuclei. Ornithine decarboxylase and antizyme mRNAs appeared to be localized in the nerve cells. ODC antibody displayed mainly cytoplasmic staining in all brain areas. Antizyme antibody staining was mainly cytoplasmic in the most brain areas, although predominantly nuclear staining was detected in some areas, most notably in the cerebellar cortex, anterior olfactory nucleus and frontal cortex. Our study is the first detailed and comparative analysis of ornithine decarboxylase and antizyme expression in the adult mammalian brain.

Raghavendra Rao V L, Dogan A, Bowen K K, Dempsey R J. Ornithine decarboxylase knockdown exacerbates transient focal cerebral ischemia-induced neuronal damage in rat brain. J Cereb Blood Flow Metab August 2001;21(8):945-54

Transient cerebral ischemia leads to increased expression of ornithine decarboxylase (ODC). Contradicting studies attributed neuroprotective and neurotoxic roles to ODC after ischemia. Using antisense oligonucleotides (ODNs), the current study evaluated the functional role of ODC in the process of neuronal damage after transient focal cerebral ischemia induced by middle cerebral artery occlusion (MCAO) in spontaneously hypertensive rats. Transient MCAO significantly increased the ODC immunoreactive protein levels and catalytic activity in the ipsilateral cortex, which were completely prevented by the infusion of antisense ODN specific for ODC. Transient MCAO in rats infused with ODC antisense ODN increased the infarct volume, motor deficits, and mortality compared with the sense or random ODN-infused controls. Results of the current study support a neuroprotective or recovery role, or both, for ODC after transient focal ischemia.

Farooqui A A, Yi Ong W, Lu X R, Halliwell B, Horrocks L A. Neurochemical consequences of kainate-induced toxicity in brain: involvement of arachidonic acid release and prevention of toxicity by phospholipase A(2) inhibitors. Brain Res Brain Res Rev December 2001;38(1-2):61-78

In kainate-induced neurotoxicity, the stimulation of kainate receptors results in the activation of phospholipase A(2) and a rapid release of arachidonic acid from neural membrane glycerophospholipids. This process raises arachidonic acid levels and produces alterations in membrane fluidity and permeability. These result in calcium influx and stimulation of lipolysis and proteolysis, production of lipid peroxides, depletion of ATP, and loss of reduced glutathione. As well as the above neurochemical changes, stimulation of ornithine decarboxylase, altered activities of protein kinase C isozymes, and expression of immediate early genes, cytokines, growth factors, and heat shock proteins have also been reported. Kainate-induced stimulation of arachidonic acid release, calcium influx, accumulation of lipid peroxides and products of their decomposition, especially 4-hydroxynonenal (4-HNE), along with alterations in cellular redox state and ATP depletion may play important roles in kainate-induced cell death. Thus the consequences of altered glycerophospholipid metabolism in kainate-induced neurotoxicity can lead to cell death. Kainate-induced neurotoxicity initiates apoptotic as well as necrotic cell death depending upon the intensity of oxidative stress and abnormality in mitochondrial function. Other neurochemical changes may be related to synaptic reorganization following kainate-induced seizures and may be involved in recapitulation of hippocampal development and synaptogenesis.

Panel 4D Summary: Ag3148: The NOV34a transcript is expressed in activated and differentiated T Cells, LPS activated macrophages and dendritic cells. In addition, TNF alpha appears to induce expression in epithelial cells, keratinocytes, and fibroblasts. Blocking of ornithine decarboxylase by drugs has shown to block respiratory burst in response to specific stimuli (see reference). Therefore, therapeutics designed with the protein encoded by this transcript may alter activation of PMNs and macrophages and be important in the treatment of inflammatory diseases such as inflammatory bowel disease, asthma, arthritis and psoriasis.

### REFERENCES

Walters J D, Cario A C, Danne M M, Marucha P T. An inhibitor of ornithine decarboxylase antagonizes superoxide generation by primed human polymorphonuclear leukocytes. J Inflamm 1998;48(1):40-6

Tumor necrosis factor-alpha (TNF-alpha) induces a rapid increase in polymorphonuclear leukocyte (PMN) polyamine content which appears to be required for optimal priming of the respiratory burst. The objective of the present study was to determine whether inhibition of polyamine biosynthesis modifies PMN responses to lipopolysaccharide (LPS), granulocyte-macrophage colony-stimulating factor (GM-CSF), or granulocyte colony-stimulating factor (G-CSF). Treatment with alpha-difluoromethylornithine (DFMO), a selective inhibitor of the rate-limiting biosynthetic enzyme ornithine decarboxylase, produced dose-dependent inhibition of the respiratory burst in PMNs that were primed by these agents and subsequently activated by formyl-Met-Leu-Phe (fMLP). However, DFMO did not significantly inhibit fMLP-stimulated superoxide generation or alter the induction of PMN adhesion and interleukin-1 beta (IL-1 beta) mRNA expression by LPS or GM-CSF. Antagonism of priming by DFMO correlated with a dose-dependent attenuation of fMLP-induced intracellular Ca2+ mobilization (r>or =0.96). Since Ca2+ plays an important role in modulating the respiratory burst in primed PMNs, this could, in part, account for the selective effects of DFMO.

PMID: 9368191

Kaczmarek L, Kaminska B, Messina L, Spampinato G, Arcidiacono A, Malaguarnera L, Messina A.Inhibitors of polyamine biosynthesis block tumor necrosis factor-induced activation of macrophages. Cancer Res Apr. 1, 1992;52(7):18914

The activation of polyamine biosynthesis, dependent on increased gene expression of ornithine decarboxylase, has been found to play an important role in the control of cell proliferation and differentiation. In this report it has been found that accumulation of ornithine decarboxylase mRNA also follows stimulation of human monocytes/macrophages by tumor necrosis factor. Human recombinant tumor necrosis factor (100 units/ml) also evoked an enhanced respiratory burst of macrophages. The respiratory burst response was inhibited in a dose-dependent manner with difluoromethylornithine, an inhibitor of ornithine decarboxylase, and methylglyoxal-bis(guanylhydrazone), an inhibitor of the formation of spermidine and spermine. The data presented in this paper suggest that polyamines may play a functional role in tumor necrosis factor-driven macrophage activation, and they are discussed in the context of their possible use as inhibitors of polyamine metabolism in tumor chemotherapy.

PMID: 1312903

Panel CNS_{—}1 Summary: Ag3148 This panel confirms the expression of the NOV34a gene in the CNS. See panel CNS_Neurodegeneration for a discussion of utility of this gene in the central nervous system.

W. NOV35—CG57339-01: Short Chain Dehydrogenaselreductase-Like Protein

Expression of the NOV35 gene was assessed using the primer-probe set Ag3203, described in Table WA. Results of the RTQ-PCR runs are shown in Tables WB, WC and WD.

TABLE WA

Probe Name Ag3203

| | | | | |
|---|---|---|---|---|
| | | | | SEQ ID |
| Primers | Sequences | Length | Start Position | NO: |

| | | | | |
|---|---|---|---|---|
| Forward | 5′-tccttcccactagacaacttga-3′ | 22 | 236 | 539 |
| Probe | TET-5′-tcctagcctatagctactcttccgttcca-3′-TAMRA | 29 | 268 | 540 |
| Reverse | 5′-atcagagcaggaaaccaagaag-3′ | 22 | 297 | 541 |

TABLE WB
CNS_neurodegeneration_v1.0

| | Rel. Exp. (%) Ag3203, | | Rel. Exp. (%) Ag3203, |
|---|---|---|---|
| Tissue Name | Run 209861768 | Tissue Name | Run 209861768 |
| AD 1 Hippo | 35.1 | Control (Path) 3 | 3.4 |
| | | Temporal Ctx | |
| AD 2 Hippo | 48.6 | Control (Path) 4 | 72.7 |
| | | Temporal Ctx | |
| AD 3 Hippo | 18.2 | AD 1 Occipital Ctx | 35.4 |
| AD 4 Hippo | 15.6 | AD 2 Occipital Ctx | 0.0 |
| | | (Missing) | |
| AD 5 Hippo | 96.6 | AD 3 Occipital Ctx | 32.3 |
| AD 6 Hippo | 65.1 | AD 4 Occipital Ctx | 22.7 |
| Control 2 Hippo | 33.9 | AD 5 Occipital Ctx | 63.7 |
| Control 4 Hippo | 57.4 | AD 6 Occipital Ctx | 21.8 |
| Control (Path) 3 | 9.6 | Control 1 Occipital | 0.0 |
| Hippo | | Ctx | |
| AD 1 Temporal Ctx | 34.9 | Control 2 Occipital | 66.9 |
| | | Ctx | |
| AD 2 Temporal Ctx | 42.0 | Control 3 Occipital | 71.2 |
| | | Ctx | |
| AD 3 Temporal Ctx | 16.0 | Control 4 Occipital | 9.5 |
| | | Ctx | |
| AD 4 Temporal Ctx | 34.2 | Control (Path) 1 | 100.0 |
| | | Occipital Ctx | |
| AD 5 Inf Temporal | 77.4 | Control (Path) 2 | 23.0 |
| Ctx | | Occipital Ctx | |
| AD 5 Sup Temporal | 58.6 | Control (Path) 3 | 3.4 |
| Ctx | | Occipital Ctx | |
| AD 6 Inf Temporal | 71.7 | Control (Path) 4 | 52.9 |
| Ctx | | Occipital Ctx | |
| AD 6 Sup Temporal | 70.2 | Control 1 Parietal Ctx | 16.6 |
| Ctx | | | |
| Control 1 Temporal | 15.0 | Control 2 Parietal Ctx | 84.7 |
| Ctx | | | |
| Control 2 Temporal | 46.0 | Control 3 Parietal Ctx | 50.0 |
| Ctx | | | |
| Control 3 Temporal | 49.3 | Control (Path) 1 | 56.6 |
| Ctx | | Parietal Ctx | |
| Control 3 Temporal | 13.1 | Control (Path) 2 | 57.0 |
| Ctx | | Parietal Ctx | |
| Control (Path) 1 | 45.1 | Control (Path) 3 | 5.6 |
| Temporal Ctx | | Parietal Ctx | |
| Control (Path) 2 | 45.1 | Control (Path) 4 | 46.7 |
| Temporal Ctx | | Parietal Ctx | |

TABLE WC
Panel 1.3D

| | Rel. Exp. (%) Ag3203, | | Rel. Exp. (%) Ag3203, |
|---|---|---|---|
| Tissue Name | Run 167994666 | Tissue Name | Run 167994666 |
| Liver adenocarcinoma | 0.0 | Kidney (fetal) | 100.0 |
| Pancreas | 0.0 | Renal ca. 786-0 | 3.4 |
| Pancreatic ca. CAPAN2 | 0.0 | Renal ca. A498 | 3.2 |
| Adrenal gland | 1.3 | Renal ca. RXF 393 | 3.2 |
| Thyroid | 9.4 | Renal ca. ACHN | 3.0 |
| Salivary gland | 4.6 | Renal ca. UO-31 | 14.6 |
| Pituitary gland | 29.7 | Renal ca. TK-10 | 6.9 |
| Brain (fetal) | 47.6 | Liver | 5.8 |
| Brain (whole) | 55.5 | Liver (fetal) | 6.7 |
| Brain (amygdala) | 30.1 | Liver ca. (hepatoblast) | 3.7 |
| | | HepG2 | |
| Brain (cerebellum) | 31.6 | Lung | 12.1 |
| Brain (hippocampus) | 26.6 | Lung (fetal) | 53.6 |
| Brain (substantia nigra) | 44.8 | Lung ca. (small cell) | 5.3 |
| | | LX-1 | |
| Brain (thalamus) | 44.8 | Lung ca. (small cell) | 2.9 |
| | | NCI-H69 | |
| Cerebral Cortex | 45.7 | Lung ca. (s. cell var.) | 49.3 |
| | | SHP-77 | |
| Spinal cord | 31.0 | Lung ca. (large | 0.0 |
| | | cell) NCI-H460 | |
| glio/astro U87-MG | 12.9 | Lung ca. (non-sm. cell) | 41.5 |
| | | A549 | |
| glio/astro U-118-MG | 5.8 | Lung ca (non-s. cell) | 32.5 |
| | | NCI-H23 | |
| astrocytoma SW1783 | 21.9 | Lung ca. (non-s. cell) | 7.5 |
| | | HOP-62 | |
| neuro*; met SK-N-AS | 0.0 | Lung ca. (non-s. cl) | 24.0 |
| | | NCI-H522 | |
| astrocytoma SF-539 | 1.5 | Lung ca. (squam.) SW | 15.6 |
| | | 900 | |
| astrocytoma SNB-75 | 12.0 | Lung ca. (squam.) | 11.9 |
| | | NCI-H596 | |
| glioma SNB-19 | 1.3 | Mammary gland | 37.1 |
| glioma U251 | 15.8 | Breast ca.* (pl. ef) | 16.7 |
| | | MCF-7 | |
| glioma SF-295 | 17.8 | Breast ca.* (pl. ef) | 1.9 |
| | | MDA-MB-231 | |
| Heart (fetal) | 14.2 | Breast ca.* (pl. ef) | 31.4 |
| | | T47D | |
| Heart | 0.8 | Breast ca. BT-549 | 6.6 |
| Skeletal muscle (fetal) | 15.9 | Breast ca. MDA-N | 0.0 |
| Skeletal muscle | 10.2 | Ovary | 39.8 |
| Bone marrow | 9.8 | Ovarian ca. OVCAR-3 | 10.2 |
| Thymus | 21.3 | Ovarian ca. OVCAR-4 | 3.0 |
| Spleen | 37.4 | Ovarian ca. OVCAR-5 | 55.1 |
| Lymph node | 18.9 | Ovarian ca. OVCAR-8 | 30.1 |
| Colorectal | 2.5 | Ovarian ca. IGROV-1 | 3.1 |
| Stomach | 17.6 | Ovarian ca* (ascites) | 13.4 |
| | | SK-OV-3 | |
| Small intestine | 4.2 | Uterus | 26.8 |
| colon ca. SW480 | 6.9 | Placenta | 0.0 |
| Colon ca.* | 39.0 | Prostate | 15.2 |
| SW620 (SW480 met) | | | |
| Colon ca. HT29 | 1.4 | Prostate ca* (bone | 9.3 |
| | | met) PC-3 | |
| Colon ca. HCT-116 | 3.1 | Testis | 95.3 |
| Colon ca. CaCo-2 | 0.0 | Melanoma Hs688(A).T | 6.5 |
| Colon ca. | 2.5 | Melanoma* (met) | 12.7 |
| tissue (ODO3866) | | Hs688(B).T | |
| Colon ca. HCC-2998 | 5.3 | Melanoma UACC-62 | 2.7 |
| Gastric ca.* (liver met) | 15.0 | Melanoma M14 | 0.0 |
| NCI-N87 | | | |
| Bladder | 9.0 | Melanoma LOX IMVI | 0.0 |
| Trachea | 12.3 | Melanoma* (met) SK- | 8.5 |
| | | MEL-5 | |
| Kidney | 17.8 | Adipose | 0.0 |

TABLE WD
Panel 4D

| | Rel. Exp. (%) Ag3203, | | Rel. Exp. (%) Ag3203, |
|---|---|---|---|
| Tissue Name | Run 164389445 | Tissue Name | Run 164389445 |
| Secondary Th1 act | 0.0 | HUVEC IL-1beta | 9.2 |
| Secondary Th2 act | 10.2 | HUVEC IFN gamma | 6.5 |
| Secondary Tr1 act | 6.2 | HUVEC TNF alpha + IFN | 0.0 |
| | | gamma | |
| Secondary Th1 rest | 4.4 | HUVEC TNF alpha + IL4 | 0.0 |
| Secondary Th2 rest | 0.0 | HUVEC IL-11 | 6.8 |
| Secondary Tr1 rest | 16.4 | Lung Microvascular EC none | 0.0 |
| Primary Th1 act | 6.4 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 71.7 | Bronchial epithelium | 13.3 |
| | | TNFalpha + IL1beta | |
| Primary Th2 rest | 8.6 | Small airway epithelium | 0.0 |
| | | none | |
| Primary Tr1 rest | 26.1 | Small airway epithelium | 12.7 |
| | | TNFalpha + IL-1beta | |
| CD45RA CD4 | 13.6 | Coronery artery SMC rest | 23.5 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 12.2 | Astrocytes rest | 0.0 |
| Secondary CD8 | 12.2 | Astrocytes TNFalpha + IL- | 0.0 |
| lymphocyte rest | | 1beta | |
| Secondary CD8 | 0.0 | KU-812 (Basophil) rest | 7.7 |
| lymphocyte act | | | |
| CD4 lymphocyte none | 8.2 | KU-812 (Basophil) | 15.9 |
| | | PMA/ionomycin | |
| 2ry Th1/Th2/Tr1_anti- | 19.2 | CCD1106 (Keratinocytes) | 33.7 |
| CD95 CH11 | | none | |
| LAK cells rest | 22.2 | CCD1106 (Keratinocytes) | 7.3 |
| | | TNFalpha + IL-1beta | |
| LAK cells IL-2 | 27.7 | Liver cirrhosis | 36.3 |
| LAK cells IL-2 + IL-12 | 0.0 | Lupus kidney | 5.0 |
| LAK cells 1L-2 + IFN | 19.1 | NCI-H292 none | 31.0 |
| gamma | | | |
| LAK cells IL-2 + IL-18 | 0.0 | NCI-H292 IL-4 | 14.1 |
| LAK cells | 0.0 | NCI-H292 IL-9 | 13.2 |
| PMA/ionomycin | | | |
| NK Cells IL-2 rest | 18.9 | NCI-H292 IL-13 | 3.3 |
| Two Way MLR 3 day | 10.1 | NCI-H292 IFN gamma | 6.3 |
| Two Way MLR 5 day | 9.1 | HPAEC none | 13.3 |
| Two Way MLR 7 day | 9.3 | HPAEC TNF alpha + IL-1 | 0.0 |
| | | beta | |
| PBMC rest | 0.0 | Lung fibroblast none | 55.9 |
| PBMC PWM | 36.9 | Lung fibroblast TNF alpha + | 49.0 |
| | | IL-1 beta | |
| PBMC PHA-L | 9.2 | Lung fibroblast IL-4 | 44.1 |
| Ramos (B cell) none | 18.3 | Lung fibroblast IL-9 | 43.5 |
| Ramos (B cell) | 48.3 | Lung fibroblast IL-13 | 49.0 |
| ionomycin | | | |
| B lymphocytes PWM | 75.8 | Lung fibroblast IFN gamma | 57.0 |
| B lymphocytes CD40L | 71.2 | Dermal fibroblast CCD1070 | 23.7 |
| and IL-4 | | rest | |
| EOL-1 dbcAMP | 0.0 | Dermal fibroblast CCD1070 | 60.7 |
| | | TNF alpha | |
| EOL-1 dbcAMP | 0.0 | Dermal fibroblast CCD1070 | 54.0 |
| PMA/ionomycin | | IL-1 beta | |
| Dendritic cells none | 6.1 | Dermal fibroblast IFN | 65.1 |
| | | gamma | |
| Dendritic cells LPS | 0.0 | Dermal fibroblast IL-4 | 52.1 |
| Dendritic cells anti- | 4.9 | IBD Colitis 2 | 0.0 |
| CD40 | | | |
| Monocytes rest | 13.1 | IBD Crohn's | 0.0 |
| Monocytes LPS | 6.6 | Colon | 100.0 |
| Macrophages rest | 0.0 | Lung | 45.7 |
| Macrophages LPS | 12.8 | Thymus | 98.6 |
| HUVEC none | 14.0 | Kidney | 47.6 |
| HUVEC starved | 0.0 | | |

CNS_neurodegeneration_v1.0 Summary: Ag3203 The NOV35 gene is not found to be differentially regulated in Alzheimer's disease; however a close homolog of this gene has been shown to mediate neurotoxicity via amyloid beta binding. Therefore, the NOV35 gene may be an excellent drug target for the treatment of Alzheimer's disease, specifically for blocking amyloid beta induced neuronal death.

### REFERENCES

He X Y, Schulz H, Yang SY.A human brain L-3-hydroxyacyl-coenzyme A dehydrogenase is identical to an amyloid beta-peptide-binding protein involved in Alzheimer's disease. J Biol Chem Apr. 24, 1998;273(17):10741-6

A novel L-3-hydroxyacyl-CoA dehydrogenase from human brain has been cloned, expressed, purified, and characterized. This enzyme is a homotetramer with a molecular mass of 108 kDa. Its subunit consists of 261 amino acid residues and has structural features characteristic of short chain dehydrogenases. It was found that the amino acid sequence of this human brain enzyme is identical to that of an endoplasmic reticulum amyloid beta-peptide-binding protein (ERAB), which mediates neurotoxicity in Alzheimer's disease (Yan, S. D., Fu, J., Soto, C., Chen, X., Zhu, H., Al-Mohanna, F., Collison, K., Zhu, A., Stem, E., Saido, T., Tohyama, M., Ogawa, S., Roher, A., and Stem, D. (1997) Nature 389, 689-695). The purification of human brain short chain L-3-hydroxyacyl-CoA dehydrogenase made it possible to characterize the structural and catalytic properties of ERAB. This NAD+-dependent dehydrogenase catalyzes the reversible oxidation of L-3-hydroxyacyl-CoAs to form 3-ketoacyl-CoAs, but it does not act on the D-isomers. The catalytic rate constant of the purified enzyme was estimated to be 37 s-1 with apparent Km values of 89 and 20 &mgr;M for acetoacetyl-CoA and NADH, respectively. The activity ratio of this enzyme for substrates with chain lengths of C4, C8, and C 16 was approximately 1:2:2. The human short chain L-3-hydroxyacyl-CoA dehydrogenase gene is organized into six exons and five introns and maps to chromosome Xp11.2. The amino-terminal NAD-binding region of the dehydrogenase is 10 encoded by the first three exons, whereas the other exons code for the carboxyl-terminal substrate-binding region harboring putative catalytic residues. The results of this study lead to the conclusion that ERAB involved in neuronal dysfunction is encoded by the human short chain L-3-hydroxyacyl-CoA dehydrogenase gene

Panel 1.3D Summary: Ag3203 Highest expression of the NOV35 gene is seen in the fetal kidney (CT=32). In addition, significant levels of expression are also seen in cell lines derived from ovarian, lung and colon cancers. Thus, expression of this gene could be used to differentiate between these samples and other samples on this panel and as a marker to detect the presence of these cancers. Furthermore, therapeutic modulation of the expression or function of this gene may be effective in the treatment of ovarian, lung and colon cancers.

Among metabolic tissues, the NOV35 gene has a low level of expression in the pituitary. Therefore, this gene product may be a small molecule target for the treatment of diseases of the pituitary, including pituitary adenomas and multiple endocrine neoplasia.

In addition, expression in the brain confirms the expression of this gene in the CNS. See panel CNS_Neurodegeneration for a discussion of utility of this gene in the central nervous system.

Panel 4D Summary: Ag3203 The expression of the NOV35 transcript is highest in colon and thymus. This gene is also expressed in fibroblasts, B cells and Th1 cells. Thus, the transcript or the protein it encodes could be used as a marker for these tissues. Additionally, therapeutics designed with the transcript encoded by this protein could be used for maintaining normal homeostasis in the colon and thymus.

X. NOV36—CG57341-01: Short Chain Dehydrogenase/Reductase

Expression of the NOV36 gene was assessed using the primer-probe set Ag3204, described in Table XA. Results of the RTQ-PCR runs are shown in Tables XB, XC and XD.

TABLE XA

Probe Name Ag3204

SEQ ID

| | | | | |
|---|---|---|---|---|
| Primers | Sequences | Length | Start Position | NO: |

| | | | | |
|---|---|---|---|---|
| Forward | 5′-ggactttgatcccctacagatg-3′ | 22 | 170 | 542 |
| Probe | TET-5′-tcaaatgaagaggacatcctctccat-3′-TAMRA | 26 | 199 | 543 |
| Reverse | 5′-ctgagaacggatagctgagaac-3′ | 22 | 225 | 544 |

TABLE XB
CNS_neurodegeneration_v1.0

| | Rel. Exp. (%) Ag3204, | | Rel. Exp. (%) Ag3204, |
|---|---|---|---|
| Tissue Name | Run 209861769 | Tissue Name | Run 209861769 |
| AD 1 Hippo | 3.9 | Control (Path) 3 | 3.7 |
| | | Temporal Ctx | |
| AD 2 Hippo | 15.3 | Control (Path) 4 | 25.5 |
| | | Temporal Ctx | |
| AD 3 Hippo | 3.3 | AD 1 Occipital Ctx | 7.2 |
| AD 4 Hippo | 3.9 | AD 2 Occipital Ctx | 0.0 |
| | | (Missing) | |
| AD 5 hippo | 67.4 | AD 3 Occipital Ctx | 3.9 |
| AD 6 Hippo | 20.9 | AD 4 Occipital Ctx | 14.7 |
| Control 2 Hippo | 24.7 | AD 5 Occipital Ctx | 16.8 |
| Control 4 Hippo | 5.4 | AD 6 Occipital Ctx | 64.2 |
| Control (Path) 3 Hippo | 3.9 | Control 1 Occipital | 5.1 |
| | | Ctx | |
| AD 1 Temporal Ctx | 7.5 | Control 2 Occipital | 100.0 |
| | | Ctx | |
| AD 2 Temporal Ctx | 10.5 | Control 3 Occipital | 18.6 |
| | | Ctx | |
| AD 3 Temporal Ctx | 2.7 | Control 4 Occipital | 5.3 |
| | | Ctx | |
| AD 4 Temporal Ctx | 12.8 | Control (Path) 1 | 78.5 |
| | | Occipital Ctx | |
| AD 5 Inf Temporal | 57.0 | Control (Path) 2 | 12.1 |
| Ctx | | Occipital Ctx | |
| AD 5 SupTemporal | 22.8 | Control (Path) 3 | 3.3 |
| Ctx | | Occipital Ctx | |
| AD 6 Inf Temporal Ctx | 26.2 | Control (Path) 4 | 22.5 |
| | | Occipital Ctx | |
| AD 6 Sup Temporal | 30.6 | Control 1 Parietal | 7.1 |
| Ctx | | Ctx | |
| Control 1 Temporal | 7.7 | Control 2 Parietal | 29.1 |
| Ctx | | Ctx | |
| Control 2 Temporal | 48.0 | Control 3 Parietal | 23.7 |
| Ctx | | Ctx | |
| Control 3 Temporal | 17.1 | Control (Path) 1 | 92.7 |
| Ctx | | Parietal Ctx | |
| Control 4 Temporal | 4.6 | Control (Path) 2 | 24.5 |
| Ctx | | Parietal Ctx | |
| Control (Path) 1 | 55.1 | Control (Path) 3 | 5.3 |
| Temporal Ctx | | Parietal Ctx | |
| Control (Path) 2 | 39.5 | Control (Path) 4 | 44.1 |
| Temporal Ctx | | Parietal Ctx | |

TABLE XC
Panel 1.3D

| | Rel. Exp. (%) Ag3204, | | Rel. Exp. (%) Ag3204, |
|---|---|---|---|
| Tissue Name | Run 167994669 | Tissue Name | Run 167994669 |
| Liver adenocarcinoma | 23.8 | Kidney (fetal) | 23.2 |
| Pancreas | 9.9 | Renal ca. 786-0 | 7.0 |
| Pancreatic ca. CAPAN2 | 9.6 | Renal ca. A498 | 11.7 |
| Adrenal gland | 3.8 | Renal ca. RXF 393 | 8.8 |
| Thyroid | 10.0 | Renal ca. ACHN | 9.7 |
| Salivary gland | 5.9 | Renal ca. UO-31 | 2.6 |
| Pituitary gland | 10.5 | Renal ca. TK-10 | 22.8 |
| Brain (fetal) | 14.0 | Liver | 7.8 |
| Brain (whole) | 46.3 | Liver (fetal) | 8.2 |
| Brain (amygdala) | 16.8 | Liver ca. (hepatoblast) | 36.9 |
| | | HepG2 | |
| Brain (cerebellum) | 22.7 | Lung | 2.2 |
| Brain (hippocampus) | 18.9 | Lung (fetal) | 5.6 |
| Brain (substantia nigra) | 20.3 | Lung ca. (small cell) | 19.1 |
| | | LX-1 | |
| Brain (thalamus) | 26.6 | Lung ca. (small cell) | 6.1 |
| | | NCI-H69 | |
| Cerebral Cortex | 66.4 | Lung ca. (s. cell var.) | 75.3 |
| | | SHP-77 | |
| Spinal cord | 6.9 | Lung ca. (large | 1.5 |
| | | cell) NCI-H460 | |
| glio/astro U87-MG | 11.8 | Lung ca. (non-sm. cell) | 13.4 |
| | | A549 | |
| glio/astro U-118-MG | 5.0 | Lung ca. (non-s. cell) | 5.0 |
| | | NCI-H23 | |
| astrocytoma SW1783 | 23.7 | Lung ca. (non-s. cell) | 10.7 |
| | | HOP-62 | |
| neuro*; met SK-N-AS | 12.9 | Lung ca. (non-s. cl) | 46.3 |
| | | NCI-H522 | |
| astrocytoma SF-539 | 5.4 | Lung ca. (squam.) SW | 4.8 |
| | | 900 | |
| astrocytoma SNB-75 | 9.4 | Lung ca. (squam.) | 23.5 |
| | | NCI-H596 | |
| glioma SNB-19 | 13.9 | Mammary gland | 16.5 |
| glioma U251 | 28.5 | Breast ca.* (pl. ef) | 1.5 |
| | | MCF-7 | |
| glioma SF-295 | 21.9 | Breast ca.* (pl. ef) | 13.0 |
| | | MDA-MB-231 | |
| Heart (fetal) | 21.9 | Breast ca.* (pl. ef) | 35.6 |
| | | T47D | |
| Heart | 9.7 | Breast ca. BT-549 | 5.1 |
| Skeletal muscle (fetal) | 13.3 | Breast ca. MDA-N | 12.0 |
| Skeletal muscle | 18.8 | Ovary | 8.1 |
| Bone marrow | 5.2 | Ovarian ca. OVCAR-3 | 10.2 |
| Thymus | 4.7 | Ovarian ca. OVCAR-4 | 15.3 |
| Spleen | 3.1 | Ovarian ca. OVCAR-5 | 46.0 |
| Lymph node | 6.5 | Ovarian ca. OVCAR-8 | 3.5 |
| Colorectal | 69.7 | Ovarian ca. IGROV-1 | 4.4 |
| Stomach | 8.7 | Ovarian ca* (ascites) | 45.4 |
| | | SK-OV-3 | |
| Small intestine | 77.4 | Uterus | 2.6 |
| Colon ca. SW480 | 26.4 | Placenta | 0.6 |
| Colon ca.* | 47.3 | Prostate | 5.8 |
| SW620 (SW480 met) | | | |
| Colon ca. HT29 | 47.0 | Prostate ca.* (bone | 4.5 |
| | | met) PC-3 | |
| Colon ca. HCT-116 | 14.0 | Testis | 7.9 |
| Colon ca. CaCo-2 | 100.0 | Melanoma Hs688(A).T | 0.9 |
| Colon ca. | 4.9 | Melanoma* (met) | 0.8 |
| tissue (ODO3866) | | Hs688(B).T | |
| Colon ca. HCC-2998 | 28.1 | Melanoma UACC-62 | 13.0 |
| Gastric ca.* (liver met) | 18.6 | Melanoma M14 | 3.2 |
| NCI-N87 | | | |
| Bladder | 5.3 | Melanoma LOX IMVI | 18.2 |
| Trachea | 1.9 | Melanoma* (met) SK- | 15.6 |
| | | MEL-5 | |
| Kidney | 27.2 | Adipose | 6.6 |

TABLE XD
Panel 4D

| | Rel. Exp. (%) Ag3204, | | Rel. Exp. (%) Ag3204, |
|---|---|---|---|
| Tissue Name | Run 164389446 | Tissue Name | Run 164389446 |
| Secondary Th1 act | 5.0 | HUVEC IL-1beta | 1.2 |
| Secondary Th2 act | 3.3 | HUVEC IFN gamma | 2.0 |
| Secondary Tr1 act | 4.0 | HUVEC TNF alpha + IFN | 1.0 |
| | | gamma | |
| Secondary Th1 rest | 0.7 | HUVEC TNF alpha + IL4 | 2.9 |
| Secondary Th2 rest | 0.6 | HUVEC IL-11 | 2.5 |
| Secondary Tr1 rest | 0.2 | Lung Microvascular EC none | 2.9 |
| Primary Th1 act | 7.4 | Lung Microvascular EC | 2.4 |
| | | TNFalpha + IL-1beta | |
| Primary Th2 act | 4.6 | Microvascular Dermal EC | 3.6 |
| | | none | |
| Primary Tr1 act | 5.4 | Microsvasular Dermal EC | 2.8 |
| | | TNFalpha + IL-1beta | |
| Primary Th1 rest | 1.9 | Bronchial epithelium | 2.5 |
| | | TNFalpha + IL1beta | |
| Primary Th2 rest | 1.1 | Small airway epithelium | 2.9 |
| | | none | |
| Primary Tr1 rest | 1.8 | Small airway epithelium | 8.8 |
| | | TNF alpha + IL-1beta | |
| CD45RA CD4 | 1.4 | Coronery artery SMC rest | 0.6 |
| lymphocyte act | | | |
| CD45RO CD4 | 3.6 | Coronery artery SMC | 0.1 |
| lymphocyte act | | TNFalpha + IL-1beta | |
| CD8 lymphocyte act | 3.8 | Astrocytes rest | 4.8 |
| Secondary CD8 | 1.8 | Astrocytes TNFalpha + IL- | 3.6 |
| lymphocyte rest | | 1beta | |
| Secondary CD8 | 2.5 | KU-812 (Basophil) rest | 6.6 |
| lymphocyte act | | | |
| CD4 lymphocyte none | 0.6 | KU-812 (Basophil) | 10.2 |
| | | PMA/ionomycin | |
| 2ry Th1/Th2/Tr1_anti- | 0.8 | CCD1106 (Keratinocytes) | 4.0 |
| CD95 CH11 | | none | |
| LAK cells rest | 2.2 | CCD1106 (Keratinocytes) | 1.8 |
| | | TNFalpha + IL-1beta | |
| LAK cells IL-2 | 2.1 | Liver cirrhosis | 1.1 |
| LAK cells IL-2 + IL-12 | 2.4 | Lupus kidney | 0.4 |
| LAK cells IL-2 + IFN | 2.9 | NCI-H292 none | 7.0 |
| gamma | | | |
| LAK cells IL-2 + IL-18 | 2.4 | NCI-H292 IL-4 | 8.8 |
| LAK cells | 1.2 | NCI-H292 IL-9 | 7.4 |
| PMA/ionomycin | | | |
| NK Cells IL-2 rest | 1.4 | NCI-H292 IL-13 | 3.9 |
| Two Way MLR 3 day | 1.5 | NCI-H292 IFN gamma | 4.1 |
| Two Way MLR 5 day | 1.6 | HPAEC none | 1.8 |
| Two Way MLR 7 day | 1.4 | HPAEC TNF alpha + IL-1 | 2.0 |
| | | beta | |
| PBMC rest | 0.0 | Lung fibroblast none | 1.4 |
| PBMC PWM | 6.7 | Lung fibroblast TNF alpha + | 0.6 |
| | | IL-1 beta | |
| PBMC PHA-L | 3.4 | Lung fibroblast IL-4 | 2.4 |
| Ramos (B cell) none | 3.6 | Lung fibroblast IL-9 | 2.8 |
| Ramos (B cell) | 10.7 | Lung fibroblast IL-13 | 1.7 |
| ionomycin | | | |
| B lymphocytes PWM | 9.4 | Lung fibroblast IFN gamma | 1.6 |
| B lymphocytes CD40L | 2.1 | Dermal fibroblast CCD1070 | 3.8 |
| and IL-4 | | rest | |
| EOL-1 dbcAMP | 2.2 | Dermal fibroblast CCD1070 | 4.1 |
| | | TNF alpha | |
| EOL-1 dbcAMP | 0.8 | Dermal fibroblast CCD1070 | 0.4 |
| PMA/ionomycin | | IL-1 beta | |
| Dendritic cells none | 27.0 | Dermal fibroblast IFN | 1.0 |
| | | gamma | |
| Dendritic cells LPS | 28.9 | Dermal fibroblast IL-4 | 2.8 |
| Dendritic cells anti- | 41.8 | IBD Colitis 2 | 0.1 |
| CD40 | | | |
| Monocytes rest | 0.9 | IBD Crohn's | 6.0 |
| Monocytes LPS | 0.2 | Colon | 100.0 |
| Macrophages rest | 22.5 | Lung | 2.0 |
| Macrophages LPS | 0.8 | Thymus | 11.6 |
| HUVEC none | 2.8 | Kidney | 1.6 |
| HUVEC starved | 7.4 | | |

CNS_neurodegeneration_v1.0 Summary: Ag3204 The NOV36 gene is found to be significantly (p=0.0008) downregulated in the temporal cortex of Alzheimer's disease patients when compared to controls. A close homolog of this gene has been shown to mediate neurotoxicity via amyloid beta binding. The NOV36 gene may therefore be an excellent drug target for the treatment of Alzheimeres disease, specifically for blocking amyloid beta induced neuronal death. Results from a second experiment with the same probe and primer are not included. The amp plot indicates there were experimental difficulties with this run.

### REFERENCES

He X Y, Schulz H, Yang SY.A human brain L-3-hydroxyacyl-coenzyme A dehydrogenase is identical to an amyloid beta-peptide-binding protein involved in Alzheimer's disease. J Biol Chem Apr. 24, 1998;273(17):10741-6

A novel L-3-hydroxyacyl-CoA dehydrogenase from human brain has been cloned, expressed, purified, and characterized. This enzyme is a homotetramer with a molecular mass of 108 kDa. Its subunit consists of 261 amino acid residues and has structural features characteristic of short chain dehydrogenases. It was found that the amino acid sequence of this human brain enzyme is identical to that of an endoplasmic reticulum amyloid beta-peptide-binding protein (ERAB), which mediates neurotoxicity in Alzheimer's disease (Yan, S. D., Fu, J., Soto, C., Chen, X., Zhu, B., Al-Mohanna, F., Collison, K., Zhu, A., Stern, E., Saido, T., Tohyama, M., Ogawa, S., Roher, A., and Stern, D. (11997) Nature 389, 689-695). The purification of human brain short chain L-3-hydroxyacyl-CoA dehydrogenase made it possible to characterize the structural and catalytic properties of ERAB. This NAD)+-dependent dehydrogenase catalyzes the reversible oxidation of L-3-hydroxyacyl-CoAs to form 3-ketoacyl-CoAs, but it does not act on the D-isomers. The catalytic rate constant of the purified enzyme was estimated to be 37 s-1 with apparent Km values of 89 and 20 &mgr;M for acetoacetyl-CoA and NADH, respectively. The activity ratio of this enzyme for substrates with chain lengths of C4, C8, and C16 was approximately 1:2:2. The human short chain L-3-hydroxyacyl-CoA dehydrogenase gene is organized into six exons and five introns and maps to chromosome Xp11.2. The amino-terminal NAD-binding region of the dehydrogenase is encoded by the first three exons, whereas the other exons code for the carboxyl-terminal substrate-binding region harboring putative catalytic residues. The results of this study lead to the conclusion that ERAB involved in neuronal dysfunction is encoded by the human short chain L-3-hydroxyacyl-CoA dehydrogenase gene.

Panel 1.3D Summary: Ag3204 The NOV36 gene is expressed at a low level in most of the cancer cell lines and normal tissues. There appears to be significantly higher expression in colon, lung, breast and ovarian cancer cell lines with the highest expression shown by a colon cancer cell line (CT=30.94). Thus, therapeutic inhibition of the NOV36 gene product, through the use of small molecule drugs, might be of utility in the treatment of the above listed cancer types.

Among tissues with metabolic function, this gene has low levels of expression in pancreas, thyroid, pituitary, adult and fetal heart, adult and fetal liver, adult and fetal skeletal muscle, and adipose. This gene product may be a small molecule target for the treatment of metabolic and endocrine disease, including the thyroidopathies, Types 1 and 2 diabetes and obesity.

In addition, this panel confirms the expression of this gene in the CNS. See panel CNS_Neurodegeneration for a discussion of utility of this gene in the central nervous system.

Panel 4D Summary: Ag3204 The NOV36 transcript is expressed at significant levels in the colon and in some types of antigen presenting cells (APC'S) including activated dendritic cells, resting macrophages, and activated B cells. This pattern of expression suggests that the protein encoded by this transcript may be involved in gut immunity, particularly in the function or maintenance of APC's. The NOV36 transcript encodes a putative reductase. Therefore, regulation of reductase expression could function by modulating gut immunity and be important in the treatment of inflammatory bowel diseases.

### Example 4

### Differential Gene Expression in Clear Cell Renal Cell Carcinomas VS Normal Adjacent Tissues

To obtain a comprehensive profile of those genes whose expression is modulated in clear cell Renal cell carcinomas, GeneCalling™ technology, described in detail in Shimkets et al. (1999) and in U.S. Pat. No. 5,871,697, was used to distinguish the gene expression profile of clear cell Renal cell carcinoma tissues with the normal adjacent tissues, obtained from the same patient, during surgical nephrectomy. The tissues were provided to CuraGen from the NDRI under an IRB approved protocol. GeneCalling™ technology relies on Quantitative Expression Analysis to generate the gene expression profile of a given sample and then generates differential expression analysis of pair-wise comparison of these profiles to controls. The comparison in this example is a pool of all tumor tissues vs. a pool of all normal tissues. Polynucleotides exhibiting differential expression were confirmed by conducting a PCR reaction according to the GeneCalling™ protocol, with the addition of a competing unlabelled primer that prevents the amplification from being detected.

[image]

[image]

Surprisingly, several cDNA fragments from ARP were over-expressed in 9 out of 11 clear cell Renal cell carcinomas.

### Example 5

### TAQMAN™ Analysis of ARP

ARP was then subjected to Taqman™ analysis (TaqMan™ polymerase chain reaction detection; Perkin Elmer, Applied Biosystems Division, Foster City, Calif.). The specific details of the PCR reactions are as follows:

Tissues were ground to a fine powder under liquid N₂ using a motorized grinding mill (Certiprep, #6800-115) and made into lysate by addition of Trizol (Life Tech, cat.#15596-018)@1.0 ml Trizol/100 mg tissue. Total RNA was extracted from this lysate by extraction with BCP (bromochloropropane; MRC, BP-151), added in an amount equal to one tenth the volume of the Trizol lysate, followed by precipitation of total RNA from the aqueous layer by addition of an equal volume of isopropanol. The precipitate was recovered by spinning the solution at 13,200 rpm for 10 minutes in micro-centrifuges (or at 9000 rpm for 15 mins in Beckman G15 centrifuge for lysate volume>1.0 ml). The precipitates were washed once with 70% ethanol, air dried briefly and resuspended in 100 μl DEPC treated water (Ambion, cat.#9920).

To remove any genomic DNA contamination from the resulting total RNA preparations, they were treated with DNase (2 μl; 10 u/μl; Qiagen, cat.#79254) in the presence of 1×DNase buffer from Promega for 30 min at 37° C. RNA was extracted by addition of equal volumes of acid phenol: chloroform (Ambion, cat.#9720), followed by precipitation from the aqueous phase with 0.3 M sodium acetate (Fluka, cat.#71196) and two volumes of ethanol. The precipitate was recovered by spinning as above, washed once with 70% ethanol and resuspended in 50 μl DEPC treated water.

RNA was quantitated fluorometrically (Tecan SpectraFluor Plus) using a RNA specific dye, Ribogreen (Molecular Probes, Eugene Oreg.; Catalog number R-11491) according to the manufacturer's directions. The quality of the RNA was determined by running the RNA either on agarose-formaldehyde gels or RNA chips (Agilent 5064-8229) from Agilent Technology (2100 Bioanalyser).

The RNA samples for each cell or tissue were normalized according to RNA input by RNA quantification using Ribogreen (as described above) using a standard curve covering the concentration range of 1 ng/ml through 50 ng/ml RNA.

Absence of genomic DNA contamination in every RNA sample was confirmed by monitoring the expression of human polypeptide chain elongation factor-I alpha (GenBank Accession Number: E02629) and human ADP-ribosylation factor 1 (ARF1) mRNA (GenBank Accession Number: M36340) by TAQMAN®, without performing a reverse transcription step prior to the PCR cycles (minus RT-TAQMAN® assay). Ten ng of RNA (total or polyA+) were used in a 25 ul TAQMAN® reaction using probe and primer sets specific for intronless segments of human polypeptide chain elongation factor-1 alpha and human ADP-ribosylation factor 1 (ARF1) mRNA. Probe and primers sets were designed for each assay according to a proprietary software package. Reactions were carried out using the TAQMAN® universal PCR Master Mix (Applied Biosystems, Foster City, Calif., USA; cat #4304447) according to the manufacturer's protocol. Reactions were performed using 96 well optical plates and caps (Applied Biosystems, cat #403012) on an ABI Prism 7700® Sequence Detection System (Applied B iosystems) using the following parameters: 10 min at 95° C.; 15 sec at 95° C./1 min at 60° C. (40 cycles). Results were recorded as CT values (cycle at which a given sample crosses a threshold level of fluorescence) using a log scale. Any sample showing a CT value lower than 35 for any of the two tested genes were treated again with DNAse 1 following the protocol described previously.

RNA (2-10 μg total or polyA+) was converted to cDNA using Superscript II(Life Tech; cat#18064-147) and random hexamers. Reactions were performed in a volume of 20 μl and incubated for 60 mins at 42° C. to generate the single stranded cDNA (sscDNA).

sscDNA was then diluted in DEPC-water to a final concentration of 0.2 ng/μl (assuming a 1:1 RNA to cDNA conversion ratio). Five μl of sscDNA was transferred to a separate plate for the TAQMAN® reaction using probe and primer sets specific for human polypeptide chain elongation factor-1 alpha and human ADP-ribosylation factor 1 (ARF1) mRNA. TAQMAN® reactions were performed following the minus RT-TAQMAN® assay protocol described previously. Results were recorded as CT values, with the difference in RNA concentration between a given sample and the sample with the lowest CT value being represented as 2 to the power of delta CT (2^{ΔCT}). The percent relative expression is then obtained by taking the reciprocal of this RNA difference and multiplying by 100. The median CT values obtained for two housekeeping genes: human polypeptide chain elongation factor-1 alpha (hEF-1α) and human ADP-ribosylation factor 1 (hARF1) were used to normalize sscDNA samples within each panel. The concentrations of the sscDNA samples were adjusted so as to be within the median CT value, +/−one CT unit for these two housekeeping genes. After every round of sscDNA concentration adjustment, the relative gene expression for hEF-1α and hARF1 sscDNA was measured by TAQMAN® as described previously.

Normalized sscDNA (5 μl) for each sample was analyzed via TAQMAN® following the minus RT-TAQMAN® assay protocol described previously. Probes and primers were designed for each assay according to a proprietary software package using the sequence of GenBank Accession number AF153606, AF169312, or AF202636 as input. The primers and probe were designed to also specifically identify the gene of the invention irrespective of the presence of related human genes, such as splice forms, homologs and paralogs. The primers and probe are shown in Table 2.

TABLE 3
Primer-probe set 2012.

| | | | | | SEQ |
|---|---|---|---|---|---|
| | | TM | | Start | ID |
| Primers | quences | ° C. | Length | Position | NO: |

| | | | | | |
|---|---|---|---|---|---|
| Forward | 5′-AAGGCTCAGAACAGCAGGAT-3′ | 59 | 20 | 478 | 545 |
| Probe | TET-5′-CAACTCTTCCACAAGGTGGCCCAG-3′-TAMRA | 70 | 24 | 502 | 546 |
| Reverse | 5′-GCTTTGCAGATGCTGAATTC-3′ | 58.6 | 20 | 557 | 547 |

Default settings were used for reaction conditions and the following parameters were set before selecting primers: primer concentration=250 nM; primer melting temperature (Tₘ) range=58°-60° C.; primer optimal Tₘ=59° C.; maximum primer difference=2° C.; probe does not have 5′ G; probe Tₘ must be 10° C. greater than primer Tₘ; amplicon size is 75 bp to 100 bp, optimal amplicon size=80 bp. The probes selected (see below) were synthesized by Synthegen (Houston Tex., USA), Applied Biosystems (Foster City Calif., USA), or Biosearch Technologies, Inc. (Novato Calif., USA). Primers were synthesized by Life Technologies (Rockville Md., USA). Probes were purified first by anion exchange HPLC, followed by reverse phase HPLC to remove uncoupled dyes and non full length products. Primers were fully de-protected, and desalted using a C-18 spin-column. All TAQMAN® reactions were performed using 250 nM of probe and 1.125 μM of reverse and forward primers.

RTQ-PCR Panel 1 Description

This 96 well plate (2 control wells, 94 test samples) panel and its variants (Panel 1.X, etc.) are composed of RNA/cDNA isolated from various human cell lines that have been established from human malignant tissues (Tumors). These cell lines have been extensively characterized by investigators in both academia and the commercial sector regarding their tumorgenicity, metastatic potential, drug resistance, invasive potential and other cancer-related properties. They serve as suitable tools for pre-clinical evaluation of anti-cancer agents and promising therapeutic strategies. RNA from these various human cancer cell lines was isolated and procured for CuraGen Corporation by the Developmental Therapeutic Branch (DTB) of the National Cancer Institute (USA). Basic information regarding their biological behavior, gene expression, and resistance to various cytotoxic agents are provided by the DTB (http:/ldtp.nci.nih.gov/).

In addition, RNA/cDNA was obtained from various human tissues derived from human autopsies performed on deceased elderly people or sudden death victims (accidents, etc.). These tissues were ascertained to be free of disease and were purchased from various high quality commercial sources such as Clontech, Research Genetics, and Invitrogen.

RNA integrity from all samples is controlled for quality by visual assessment of agarose gel electrophoresis using 28s and 18s ribosomal RNA staining intensity ratio as a guide (2:1 to 2.5:1 28s: 18s) and the presence of low molecular weight RNAs indicative of degradation products. Samples are quality controlled for genomic DNA contamination by reactions run in the absence of reverse transcriptase using probe and primer sets designed to amplify across the span of a single exon.

RTQ-PCR Panel 2 Description

This 96 well (2 control wells, 94 test samples) panel and its variants (Panel 2×, etc.) are composed of RNA/cDNA isolated from human tissue procured by surgeons working in close cooperation with the National Cancer Institute's Cooperative Human Tissue Network (CHTN) or the National Disease Research Initiative (NDRI). The tissues procured are derived from human malignancies and in cases were indicated many malignant tissues have “matched margins”. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery. In addition, RNA/cDNA was obtained from various human tissues derived from human autopsies performed on deceased elderly people or sudden death victims (accidents, etc.). These tissue were ascertained to be free of disease and were purchased from various high quality commercial sources such as Clontech, Research Genetics, and Invitrogen.

RNA integrity from all samples is controlled for quality by visual assessment of agarose gel electrophoresis using 28s and 18s ribosomal RNA staining intensity ratio as a guide (2:1 to 2.5:1 28s: 18s) and the presence of low molecular weight RNAs indicative of degradation products. Samples are quality controlled for genomic DNA contamination by reactions run in the absence of reverse transcriptase using probe and primer sets designed to amplify across the span of a single exon.

RTQ-PCR Panel 4 Description

This 96 well plate (2 control wells, 94 test samples) is composed of RNA (Panel 4r) or cDNA (Panel 4d) isolated from various human cell lines or tissues. Total RNA from control normal tissues: colon, and lung were purchased from Stratagene; thymus and kidney total RNA was obtained from Clontech. Total RNA from liver tissue from Cirrhosis patients and kidney from Lupus patients were obtained from Biochain. Intestinal tissue for RNA preparation from Crohns and Ulcerative colitis patients was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics and grown in the media supplied for these cell types by Clonetics. These primary cell types were activated with various cytokines or combinations of cytokines for 6 and/or 12-14 hours. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. For endothelial cells we sometimes starved the cells for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA or PWM at approximately 5 μg/ml. Samples were taken at 24, 48 and 72 hours for RNA preparation. MLR samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

To prepare monocytes, macrophages and dendritic cells, monocytes were isolated from mononuclear cells using CD14 Miltenyi Beads, +ve VS selection columns and a Vario Magnet as per the manufacturer's instructions. Monocytes were differentiated into dendritic cells by culture in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with LPS at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet as per the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and +ve selection. Then CD4RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercatoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 106 cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 AM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. We also obtained a keratinocyte line CCD106 and an airway epithelial tumor line NCI-H292 from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, we prepared RNA by lysing approximately 10⁷ cells/ml using Trizol (Gibco BRL). Briefly, 1/10 volume of Bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees

RTO-PCR-AI Comprehensive Panel v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-lanti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the Al_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (N4F)=Individual patients

Adj=Adjacent tissue

Match control=adjacent tissues

-M=Male

-F=Female

COPD=Chronic obstructive pulmonary disease

The results are shown in Tables 4-8

TABLE 4
TaqMan data for Panel 1.3

| Tissue Name | Rel. Expr. % | Tissue Name | Rel. Expr. % |
|---|---|---|---|
| Liver adenocarcinoma | 26.2 | Renal 786-0 | 0.1 |
| Heart (fetal) | 10.0 | Renal A498 | 100.0 |
| Pancreas | 4.1 | Renal RXF 393 | 4.8 |
| Pancreatic ca. CAPAN2 | 3.4 | Renal ACHN | 3.5 |
| Adrenal gland | 11.2 | Renal UO-31 | 2.0 |
| Thyroid | 13.9 | Renal TK-10 | 3.3 |
| Salivary gland | 2.9 | Liver | 8.7 |
| Pituitary gland | 2.7 | Liver (fetal) | 12.0 |
| Brain (fetal) | 4.4 | Liver (hepatoblast) HepG2 | 5.7 |
| Brain (whole) | 11.1 | Lung | 18.7 |
| Brain (amygdala) | 7.3 | Lung (fetal) | 4.4 |
| Brain (cerebellum) | 0.9 | Lung (small cell) LX-1 | 0.6 |
| Brain (hippocampus) | 21.0 | Lung (small cell) NCI-H69 | 0.6 |
| Brain (thalamus) | 8.0 | Lung (s. cell var.) SHP-77 | 1.0 |
| Cerebral Cortex | 22.9 | Lung (large cell) NCI-H460 | 1.8 |
| Spinal cord | 17.1 | Lung (non-sm. cell) A549 | 3.1 |
| Glio/astro U87-MG | 2.7 | Lung (non-s. cell) NCI-H23 | 6.3 |
| Glio/astro U-118-MG | 38.2 | Lung (non-s. cell) HOP-62 | 23.7 |
| astro SW1783 | 20.2 | Lung (non-s. cl) NCI-H522 | 10.8 |
| neuro; met SK-N-AS | 10.7 | Lung (squam.) SW 900 | 1.2 |
| astro SF-539 | 0.3 | Lung (squam.) NCI-H596 | 0.0 |
| astro SNB-75 | 15.7 | Mammary gland | 35.1 |
| Glio SNB-19 | 0.0 | Breast (pl. ef) MCF-7 | 2.6 |
| Glio U251 | 0.1 | Breast (pl. ef) MDA-MB-231 | 6.3 |
| Glio SF-295 | 4.3 | Breast (pl. ef) T47D | 8.0 |
| Heart | 2.9 | Breast BT-549 | 40.6 |
| Skeletal muscle | 2.2 | Breast MDA-N | 0.8 |
| Bone marrow | 6.7 | Ovary | 14.1 |
| Thymus | 3.7 | Ovarian OVCAR-3 | 0.4 |
| Spleen | 4.9 | Ovarian OVCAR-4 | 1.3 |
| Lymph node | 6.4 | Ovarian OVCAR-5 | 6.2 |
| Colorectal | 3.9 | Ovarian OVCAR-8 | 0.4 |
| Stomach | 5.7 | Ovarian IGROV-1 | 0.0 |
| Small intestine | 5.3 | Ovarian (ascites) SK-OV-3 | 3.5 |
| Colon SW480 | 1.3 | Uterus | 4.5 |
| Colon SW620 (SW480 met) | 0.2 | Placenta | 95.9 |
| Colon HT29 | 0.6 | Prostate | 9.3 |
| Colon HCT-116 | 2.6 | Prostate (bone met)PC-3 | 2.7 |
| Colon CaCo-2 | 0.8 | Testis | 2.9 |
| Colon Ca tissue (ODO3866) | 23.7 | Melanoma Hs688(A).T | 4.4 |
| Colon HCC-2998 | 3.9 | Melanoma (met) Hs688(B).T | 27.7 |
| Gastric (liver met) NCI-N87 | 6.6 | Melanoma UACC-62 | 0.2 |
| Bladder | 6.0 | Melanoma M14 | 0.0 |
| Trachea | 6.1 | Melanoma LOX IMVI | 1.2 |
| Kidney | 0.4 | Melanoma (met) SK-MEL-5 | 0.0 |
| Kidney (fetal) | 22.1 | Adipose | 59.9 |

TABLE 5
TaqMan data for Panel 2.

| | Rel. | | Rel. |
|---|---|---|---|
| | Expr. | | Expr. |
| Tissue Name | % | Tissue Name | % |
| Normal Colon | 1.0 | RCC 7 Margin | 0.1 |
| CCa 1 | 0.9 | RCC 8 | 0.1 |
| CCa 1 Margin | 0.6 | RCC 8 Margin | 0.9 |
| CCa 2 | 0.5 | RCC 9 | 24.7 |
| CCa 2 Margin | 0.5 | RCC 9 Margin | 1.1 |
| CCa 3 | 0.1 | Normal Uterus | 0.1 |
| CCa 3 Margin | 0.3 | UtCa 1 | 0.5 |
| CCa 4 | 0.2 | Normal Thyroid | 1.1 |
| CCa 4 Margin | 0.2 | ThyCa 1 | 1.5 |
| CCa 5 Metastasis | 1.6 | ThyCa 2 | 0.2 |
| CCa 5 Margin (Liver) | 1.9 | ThyCa 2 Margin | 0.5 |
| CCa 6 | 0.5 | Normal Breast | 0.9 |
| CCa 6 Margin (Lung) | 0.2 | BrCa 1 | 0.2 |
| Normal Prostate | 1.1 | BrCa 2 | 0.4 |
| PCa 1 | 0.2 | BrCa 3 Metastasis | 2.0 |
| PCa 1 Margin | 0.5 | BrCa 4 Metastasis | 0.5 |
| PCa 2 | 0.3 | BrCa 5 | 0.2 |
| PCa 2 Margin | 0.4 | BrCa 6 | 0.5 |
| Normal Lung | 0.3 | BrCa 6 Margin | 0.5 |
| LCa 1 Metastasis | 1.6 | BrCa 7 | 0.4 |
| LCa 1 Margin (Muscle) | 4.2 | BrCa 7 Margin | 0.3 |
| LCa 2 | 0.4 | Normal Liver | 1.8 |
| LCa 2 Margin | 0.3 | HCC 1 | 4.2 |
| LCa 3 | 2.5 | HCC 2 | 3.6 |
| LCa 3 Margin | 1.7 | HCC 3 | 2.0 |
| LCa 4 | 0.4 | HCC 4 | 7.1 |
| LCa 5 | 0.6 | HCC 4 Margin | 0.9 |
| LCa 5 Margin | 4.6 | HCC 5 | 2.8 |
| Ocular Melanoma | 0.0 | HCC 5 Margin | 1.4 |
| Metastasis | | | |
| Ocular Melanoma Margin (Liver) | 2.5 | Normal Bladder | 2.3 |
| Melanoma Metastasis | 0.6 | TCC 1 | 0.7 |
| Melanoma Margin (Lung) | 1.6 | TCC 2 | 0.4 |
| Normal Kidney | 0.1 | TCC 3 | 2.9 |
| RCC 1 | 0.5 | TCC 3 Margin | 2.7 |
| RCC 1 Margin | 0.9 | Normal Ovary | 0.4 |
| RCC 2 | 1.2 | OvCa 1 | 0.3 |
| RCC 2 Margin | 1.3 | OvCa 2 | 4.1 |
| RCC 3 | 100.0 | OvCa 2 Margin | 3.1 |
| RCC 3 Margin | 1.0 | Normal Stomach | 0.3 |
| RCC 4 | 8.1 | Normal Stomach | 0.1 |
| RCC 4 Margin | 0.6 | GaCa 1 | 0.2 |
| RCC 5 | 32.3 | GaCa 1 Margin | 0.3 |
| RCC 5 Margin | 0.2 | GaCa 2 | 0.2 |
| RCC 6 | 0.2 | GaCa 2 Margin | 0.2 |
| RCC 6 Margin | 0.2 | GaCa 3 | 0.4 |
| RCC 7 | 0.2 | RCC 7 Margin | 0.1 |

TABLE 6
TaqMan data for Panel 3.

| | Rel. | | Rel. |
|---|---|---|---|
| Tissue Name | Expr. % | Tissue Name | Expr. % |
| 94905_Daoy_Medulloblastoma/Cerebellum_s | 0.1 | 94954_Ca Ski_Cervical | 5.5 |
| scDNA | | epidermoid carcinoma | |
| | | (metastasis)_sscDNA | |
| 94906_TE671_Medulloblastom/Cerebellum_s | 3.2 | 94955_ES-2_Ovarian clear | 1.5 |
| scDNA | | cell carcinoma_sscDNA | |
| 94907_D283 | 0.4 | 94957_Ramos/6h_stim_″; | 0.0 |
| Med_Medulloblastoma/Cerebellum_sscDNA | | Stimulated with | |
| | | PMA/ionomycin 6h_sscDNA | |
| 94908_PFSK-1_Primitive | 3.2 | 94958_Ramos/14h stim_″; | 0.0 |
| Neuroectodermal/Cerebellum_sscDNA | | Stimulated with | |
| | | PMA/ionomycin | |
| | | 14h_sscDNA | |
| 94909_XF-498_CNS_sscDNA | 3.1 | 94962_MEG-01_Chronic | 0.9 |
| | | myelogenous leukemia | |
| | | (megokaryoblast)_sscDNA | |
| 94910_SNB-78_CNS/glioma_sscDNA | 7.6 | 94963_Raji_Burkitt's | 0.2 |
| | | lymphoma_sscDNA | |
| 94911_SF-268_CNS/glioblastoma_sscDNA | 2.9 | 94964_Daudi_Burkitt's | 0.3 |
| | | lymphoma_sscDNA | |
| 94912_T98G_Glioblastoma_sscDNA | 0.2 | 94965_U266_B-cell | 1.1 |
| | | plasmacytoma/myeloma_ssc | |
| | | DNA | |
| 96776_SK-N-SH_Neuroblastoma | 11.7 | 94968_CA46_Burkitt's | 0.0 |
| (metastasis)_sscDNA | | lymphoma_sscDNA | |
| 94913_SF-295_CNS/glioblastoma_sscDNA | 1.4 | 94970_RL_non-Hodgkin's | 0.0 |
| | | B-cell lymphoma_sscDNA | |
| 94914_CerebeIlum_sscDNA | 5.5 | 94972_JM1_pre-B-cell | 0.2 |
| | | lymphoma/leukemia_sscDNA | |
| 96777_Cerebellum_sscDNA | 3.4 | 94973_Jurkat_T cell | 1.2 |
| | | leukemia_sscDNA | |
| 94916_NCI-H292_Mucoepidermoid lung | 8.2 | 94974_TF- | 0.3 |
| carcinoma_sscDNA | | 1_Erythroleukemia_sscDNA | |
| 94917_DMS-114_Small cell lung | 1.1 | 94975 HUT_78_T-cell | 0.6 |
| cancer_sscDNA | | lymphoma_sscDNA | |
| 94918_DMS-79_Small cell lung | 8.5 | 94977_U937_Histiocytic | 0.4 |
| cancer/neuroendocrine_sscDNA | | lymphoma_sscDNA | |
| 94919_NCI-H146_Small cell lung | 0.6 | 94980_KU- | 0.5 |
| cancer/neuroendocrine_sscDNA | | 812_Myelogenous | |
| | | leukemia_sscDNA | |
| 94920_NCI-H526_Small cell lung | 0.8 | 94981_769-P_Clear cell | 3.1 |
| cancer/neuroendocrine_sscDNA | | renal carcinoma_sscDNA | |
| 94921_NCI-N417_Small cell lung | 0.0 | 94983_Caki-2_Clear cell | 35.9 |
| cancer/neuroendocrine_sscDNA | | renal carcinoma_sscDNA | |
| 94923_NCI-H82_Small cell lung | 0.3 | 94984_SW 839_Clear cell | 24.5 |
| cancer/neuroendocrine_sscDNA | | renal carcinoma_sscDNA | |
| 94924_NCI-H157_Squamous cell lung cancer | 0.2 | 94986_G401_Wilms' | 0.9 |
| (metastasis)_sscDNA | | tumor_sscDNA | |
| 94925_NCI-H1155_Large cell lung | 0.5 | 94987_Hs766T_Pancreatic | 18.3 |
| cancer/neuroendocrine_sscDNA | | carcinoma (LN | |
| | | metastasis)_sscDNA | |
| 94926_NCI-H1299_Large cell lung | 37.9 | 94988_CAPAN- | 2.6 |
| cancer/neuroendocrine_sscDNA | | 1_Pancreatic | |
| | | adenocarcinoma (liver | |
| | | metastasis)_sscDNA | |
| 94927_NCI-H727_Lung carcinoid_sscDNA | 0.7 | 94989_SU86.86_Pancreatic | 0.2 |
| | | carcinoma (liver | |
| | | metastasis)_sscDNA | |
| 94928_NCI-UMC-11_Lung | 0.7 | 94990_BxPC-3_Pancreatic | 5.8 |
| carcinoid_sscDNA | | adenocarcinoma_sscDNA | |
| 94929_LX-1_Small cell lung cancer_sscDNA | 0.0 | 94991_HPAC_Pancreatic | 1.4 |
| | | adenocarcinoma_sscDNA | |
| 94930_CoIo-205_Colon cancer_sscDNA | 0.8 | 94992_MIA PaCa- | 2.5 |
| | | 2_Pancreatic | |
| | | carcinoma_sscDNA | |
| 94931_KM12_Colon cancer_sscDNA | 1.0 | 94993_CFPAC-l_Pancreatic | 3.0 |
| | | ductal | |
| | | adenocarcinoma_sscDNA | |
| 94932_KM20L2_Colon cancer_sscDNA | 0.0 | 94994_PANC-1_Pancreatic | 100.0 |
| | | epithelioid ductal | |
| | | carcinoma_sscDNA | |
| 94933_NCI-H716_Colon cancer_sscDNA | 6.1 | 94996_T24_Bladder | 49.0 |
| | | carcinma (transitional | |
| | | cell)_sscDNA | |
| 94935_SW-48_Colon | 0.3 | 94997_5637_Bladder | 0.5 |
| adenocarcinoma_sscDNA | | carcinoma_sscDNA | |
| 94936_SW1116_Colon | 0.4 | 94998_HT-1197_Bladder | 5.2 |
| adenocarcinoma_sscDNA | | carcinoma_sscDNA | |
| 94937_LS 174T_Colon | 0.2 | 94999_UM-UC-3_Bladder | 62.0 |
| adenocarcinoma_sscDNA | | carcinma (transitional | |
| | | cell)_sscDNA | |
| 94938_SW-948_Colon | 0.0 | 95000_A204_Rhabdomyosar | 0.6 |
| adenocarcinoma_sscDNA | | coma_sscDNA | |
| 94939_SW-480_Colon | 0.5 | 95001_HT- | 0.1 |
| adenocarcinoma_sscDNA | | 1080_Fibrosarcoma_sscDNA | |
| 94940_NCI-SNU-5_Gastric | 1.5 | 95002_MG- | 18.7 |
| carcinoma_sscDNA | | 63_Osteosarcoma | |
| | | (bone)_sscDNA | |
| 94941_KATO III_Gastric | 1.2 | 95003_SK-LMS- | 9.3 |
| carcinoma_sscDNA | | 1_Leiomyosarcoma | |
| | | (vulva)_sscDNA | |
| 94943_NCI-SNU-16_Gastric | 97.3 | 95004_SJRH30_Rhabdomyo | 0.4 |
| carcinoma_sscDNA | | sarcoma (met to bone | |
| | | marrow)_sscDNA | |
| 94944_NCI-SNU-1_Gastric | 1.4 | 95005_A431_Epidermoid | 0.4 |
| carcinoma_sscDNA | | carcinoma_sscDNA | |
| 94946_RF-1_Gastric | 0.0 | 95007_WM266- | 18.2 |
| adenocarcinoma_sscDNA | | 4_Melanoma_sscDNA | |
| 94947_RF-48_Gastric | 0.0 | 95010_DU 145_Prostate | 0.3 |
| adenocarcinoma_sscDNA | | carcinoma (brain | |
| | | metastasis)_sscDNA | |
| 96778_MKN-45_Gastric carcinoma_sscDNA | 3.4 | 95012_MDA-MB- | 0.0 |
| | | 468_Breast | |
| | | adenocarcinoma_sscDNA | |
| 94949_NCI-N87_Gastric carcinoma_sscDNA | 0.3 | 95013_SCC-4_Squamous | 0.0 |
| | | cell carcinoma of | |
| | | tongue_sscDNA | |
| 94951_OVCAR-5_Ovarian | 2.0 | 95014_SCC-9_Squamous | 0.7 |
| carcinoma_sscDNA | | cell carcinoma of | |
| | | tongue_sscDNA | |
| 94952_RL95-2_Uterine carcinoma_sscDNA | 1.7 | 95015_SCC-15_Squamous | 0.0 |
| | | cell carcinoma of | |
| | | tongue_sscDNA | |
| 94953_HelaS3_Cervical | 1.2 | 95017_CAL 27_Squamous | 0.0 |
| adenocarcinoma_sscDNA | | cell carcinoma of | |
| | | tongue_sscDNA | |

TABLE 7
TaqMan data for Panel 4

| | Rel. | | Rel. |
|---|---|---|---|
| | Expr. | | Expr. |
| Tissue Name | % | Tissue Name | % |
| Secondary Th1 act | 0.2 | HUVEC IL-1beta | 0.4 |
| Secondary Th2 act | 0.3 | HUVEC IFN gamma | 0.8 |
| Secondary Tr1 act | 0.6 | HUVEC TNF alpha + IFN gamma | 0.5 |
| Secondary Th1 rest | 0.1 | HUVEC TNF alpha + IL4 | 3.0 |
| Secondary Th2 rest | 0.0 | HUVEC IL-11 | 1.2 |
| Secondary Tr1 rest | 0.1 | Lung Microvascular EC none | 9.6 |
| Primary Th1 act | 0.0 | Lung Microvascular EC TNFalpha + | 11.0 |
| | | IL-1beta | |
| Primary Th2 act | 0.0 | Microvascular Dermal EC none | 16.5 |
| Primary Tr1 act | 0.1 | Microsvasular Dermal EC TNFalpha + | 9.9 |
| | | IL-1beta | |
| Primary Th1 rest | 0.1 | Bronchial epithelium TNFalpha + | 3.7 |
| | | IL1beta | |
| Primary Th2 rest | 0.1 | Small airway epithelium none | 18.6 |
| Primary Tr1 rest | 0.1 | Small airway epithelium TNFalpha + | 100.0 |
| | | IL-1beta | |
| CD45RA CD4 lymphocyte act | 0.1 | Coronery artery SMC rest | 6.8 |
| CD45RO CD4 lymphocyte act | 0.1 | Coronery artery SMC TNFalpha + | 2.1 |
| | | IL-1beta | |
| CD8 lymphocyte act | 0.1 | Astrocytes rest | 5.3 |
| Secondary CD8 lymphocyte rest | 0.3 | Astrocytes TNFalpha + IL-1beta | 8.1 |
| Secondary CD8 lymphocyte act | 0.2 | KU-812 (Basophil) rest | 0.1 |
| CD4 lymphocyte none | 0.0 | KU-812 (Basophil) PMA/ionomycin | 0.8 |
| 2ry Th1/Th2/Tr1_anti-CD95 CH11 | 0.1 | CCD1106 (Keratinocytes) none | 1.9 |
| LAK cells rest | 0.1 | CCD1106 (Keratinocytes) TNFalpha + | 2.1 |
| | | IL-1beta | |
| LAK cells IL-2 | 0.5 | Liver cirrhosis | 7.3 |
| LAK cells IL-2 + IL-12 | 0.1 | Lupus kidney | 0.1 |
| LAK cells IL-2 + IFN gamma | 0.1 | NCI-H292 none | 0.2 |
| LAK cells IL-2 + IL-18 | 0.2 | NCI-H292 IL-4 | 1.0 |
| LAK cells PMA/ionomycin | 35.9 | NCI-H292 IL-9 | 0.5 |
| NK Cells IL-2 rest | 0.1 | NCI-H292 IL-13 | 0.6 |
| Two Way MLR 3 day | 0.2 | NCI-H292 IFN gamma | 0.4 |
| Two Way MLR 5 day | 0.1 | HPAEC none | 4.8 |
| Two Way MLR 7 day | 0.1 | HPAEC TNF alpha + IL-1 beta | 6.6 |
| PBMC rest | 0.1 | Lung fibroblast none | 2.0 |
| PBMC PWM | 0.4 | Lung fibroblast TNF alpha + IL-1 | 0.7 |
| | | beta | |
| PBMC PHA-L | 1.9 | Lung fibroblast IL-4 | 14.8 |
| Ramos (B cell) none | 0.0 | Lung fibroblast IL-9 | 4.1 |
| Ramos (B cell) ionomycin | 0.1 | Lung fibroblast IL-13 | 7.0 |
| B lymphocytes PWM | 0.7 | Lung fibroblast IFN gamma | 13.0 |
| B lymphocytes CD40L and IL-4 | 0.3 | Dermal fibroblast CCD1070 rest | 2.2 |
| EOL-1 dbcAMP | 0.3 | Dermal fibroblast CCD1070 TNF | 1.1 |
| | | alpha | |
| EOL-1 dbcAMP PMA/ionomycin | 0.3 | Dermal fibroblast CCD1070 IL-1 | 0.8 |
| | | beta | |
| Dendritic cells none | 0.4 | Dermal fibroblast IFN gamma | 1.1 |
| Dendritic cells LPS | 0.8 | Dermal fibroblast IL-4 | 7.9 |
| Dendritic cells anti-CD40 | 0.3 | IBD Colitis 1 | 0.2 |
| Monocytes rest | 0.2 | IBD Colitis 2 | 0.8 |
| Monocytes LPS | 0.1 | IBD Crohn's | 2.4 |
| Macrophages rest | 0.2 | Colon | 3.8 |
| Macrophages LPS | 0.5 | Lung | 4.6 |
| HUVEC none | 1.6 | Thymus | 0.8 |
| HUVEC starved | 1.0 | Kidney | 3.6 |

TABLE 8
RTQ-PCR for panel A/I

| | Rel. Expr., % |
|---|---|
| Tissue Name | tm8144t_ag2012_b1 |
| 110967 COPD-F | 3.2 |
| 110980 COPD-F | 1.7 |
| 110968 COPD-M | 3.6 |
| 110977 COPD-M | 3.9 |
| 110989 Emphysema-F | 2.7 |
| 110992 Emphysema-F | 1.2 |
| 110993 Emphysema-F | 2.1 |
| 110994 Emphysema-F | 1.7 |
| 110995 Emphysema-F | 1.9 |
| 110996 Emphysema-F | 0.3 |
| 110997 Asthma-M | 0.8 |
| 111001 Asthma-F | 0.7 |
| 111002 Asthma-F | 1.3 |
| 111003 Atopic Asthma-F | 2.5 |
| 111004 Atopic Asthma-F | 2.5 |
| 111005 Atopic Asthma-F | 1.4 |
| 111006 Atopic Asthma-F | 0.5 |
| 111417 Allergy-M | 0.9 |
| 112347 Allergy-M | 0.1 |
| 112349 Normal Lung-F | 0.0 |
| 112357 Normal Lung-F | 5.5 |
| 112354 Normal Lung-M | 1.1 |
| 112374 Crohns-F | 0.9 |
| 112389 Match Control Crohns-F | 1.9 |
| 112375 Crohns-F | 1.0 |
| 112732 Match Control Crohns-F | 2.4 |
| 112725 Crohns-M | 0.1 |
| 112387 Match Control Crohns-M | 0.7 |
| 112378 Crohns-M | 0.1 |
| 112390 Match Control Crohns-M | 1.9 |
| 112726 Crohns-M | 3.2 |
| 112731 Match Control Crohns-M | 1.1 |
| 112380 Ulcer Col-F | 3.4 |
| 112734 Match Control Ulcer Col-F | 4.6 |
| 112384 Ulcer Col-F | 4.3 |
| 112737 Match Control Ulcer Col-F | 1.4 |
| 112386 Ulcer Col-F | 1.2 |
| 112738 Match Control Ulcer Col-F | 2.2 |
| 112381 Ulcer Col-M | 0.2 |
| 112735 Match Control Ulcer Col-M | 0.4 |
| 112382 Ulcer Col-M | 2.2 |
| 112394 Match Control Ulcer Col-M | 0.3 |
| 112383 Ulcer Col-M | 1.5 |
| 112736 Match Control Ulcer Col-M | 1.7 |
| 112423 Psoriasis-F | 1.8 |
| 112427 Match Control Psoriasis-F | 3.9 |
| 112418 Psoriasis-M | 3.2 |
| 112723 Match Control Psoriasis-M | 5.1 |
| 112419 Psoriasis-M | 3.1 |
| 112424 Match Control Psoriasis-M | 0.9 |
| 112420 Psoriasis-M | 7.2 |
| 112425 Match Control Psoriasis-M | 2.2 |
| 104689 (MF) OA Bone-Backus | 58.2 |
| 104690 (MF) Adj “Normal” Bone-Backus | 71.9 |
| 104691 (MF) OA Synovium-Backus | 29.3 |
| 104692 (BA) OA Cartilage-Backus | 81.9 |
| 104694 (BA) OA Bone-Backus | 13.5 |
| 104695 (BA) Adj “Normal” Bone-Backus | 48.6 |
| 104696 (BA) OA Synovium-Backus | 37.4 |
| 104700 (SS) OA Bone-Backus | 36.8 |
| 104701 (SS) Adj “Normal” Bone-Backus | 42.1 |
| 104702 (SS) OA Synovium-Backus | 100.0 |
| 117093 OA Cartilage Rep7 | 4.2 |
| 112672 OA Bone5 | 4.5 |
| 112673 OA Synovium5 | 1.6 |
| 112674 OA Synovial Fluid cells5 | 2.0 |
| 117100 OA Cartilage Repl4 | 1.9 |
| 112756 OA Bone9 | 1.8 |
| 112757 OA Synovium9 | 4.5 |
| 112758 OA Synovial Fluid Cells9 | 1.7 |
| 117125 RA Cartilage Rep2 | 13.6 |
| 113492 Bone2 RA | 3.0 |
| 113493 Synovium2 RA | 1.0 |
| 113494 Syn Fluid Cells RA | 2.8 |
| 113499 Cartilage4 RA | 2.4 |
| 113500 Bone4 RA | 2.6 |
| 113501 Synovium4 RA | 1.8 |
| 113502 Syn Fluid Cells4 RA | 1.5 |
| 113495 Cartilage3 RA | 1.8 |
| 113496 Bone3 RA | 1.7 |
| 113497 Synovium3 RA | 0.8 |
| 113498 Syn Fluid Cells3 RA | 3.0 |
| 117106 Normal Cartilage Rep20 | 7.3 |
| 113663 Bone3 Normal | 0.2 |
| 113664 Synovium3 Normal | 0.0 |
| 113665 Syn Fluid Cells3 Normal | 0.0 |
| 117107 Normal Cartilage Rep22 | 1.5 |
| 113667 Bone4 Normal | 0.8 |
| 113668 Synovium4 Normal | 0.8 |
| 113669 Syn Fluid Cells4 Normal | 1.7 |

ARP is overexpressed in 3/5 clear cell renal cell carcinomas, 0/2 papillary renal cell carcinomas and 0/2 uncharacterized renal cell carcinomas (panel 2D). Furthermore ARP is expressed in fetal kidney and renal cell carcinoma-derived cell lines but not in adult kidney (panel 1.3D), an indication of an oncofetal expression pattern often associated with genes involved in kidney development and organogenesis and kidney tumorgenesis.

Data from Panel 4D indicates that upon immune-stimulation of the airway epithelial cells and lung fibroblasts, ARP is expressed at increased levels. Specifically, it is show that expression of ARP in small airway epithelial cells treated with TNF alpha and IL-1 beta is up-regulated ca. 5.4 fold relative to untreated cells. In addition, expression in normal human lung fibroblast cells treated with IL-4, IL-9, IL-9, IL-13 and Interferon gamma is upregulated 7.4, 2, 3.5 and 6.5 fold, respectively, compared to that in resting cells. Finally, expression of ARP in LAK cells treated with PMA/ionomycin is upregulated over 350 fold relative to the expression in resting cells. These data indicate that ARP plays a role in inflammation related to the above cells of the pulmonary system and is thereby implicated as a target for therapeutic intervention by protein and antibody therapeutics as well as small molecule pharmaceuticals. A wholly human antibody directed at ARP, for example, may diminish the symptoms of patients with allergy, asthma or emphysema.

Studies have indicated that PMA induces down-regulation of LAK cell-mediated cytotoxicity (by inactivation of protein kinase C activity in LAK cells). The exact role of ARP is not known in LAK cells, however, based on the TaqMan data presented in present invention, ARP plays a role in inflammation and may be implicated in the ability of LAK cells to effectively destroy tumor cells as well. Therefore a therapeutic antibody directed against ARP (and thereby preventing ARP from being upregulated), may be therapeutic in treating cancer because of the resulting increased activity of LAK cells.

Data from Panel A/I illustrates that ARP transcript is highly expressed in joint tissue from Osteoarthritic patients, but not in tissue from joint tissue from normal patients. ARP is a target of peroxisome proliferator-activated receptor-gamma (PPARG) and may have a role in regulation of systemic lipid metabolism or glucose homeostasis. The data presented on the A/I panel, and from studies done with PPARG are consistent with ARP also functioning in the development and pathogenesis of osteoarthritis.

TABLE 9
SAGE Data
Hs 2613: PPAR(gamma) angiopoletin related protein
SAGE library data and reliable tag summary.
Reliable tags found in SAGE libraries:

| | Tags per | | Tag | Total |
|---|---|---|---|---|
| Library name | million | | counts | tags |
| SAGE Chem LNCaP no-DHT | 15 | 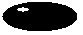 | 1 | 64631 |
| SAGE SciencePark MCF7pbk Control 0h | 16 | 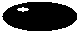 | 1 | 61079 |
| SAGE Duke post crisis fibroblasts | 13 | 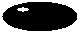 | 1 | 71792 |
| SAGE Duke 1273 | 77 | 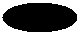 | 3 | 38836 |
| SAGE Duke thalamus | 123 | 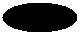 | 3 | 24371 |
| SAGE CAPAN2 | 43 | 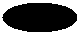 | 1 | 23222 |
| SAGE HS766T | 286 | 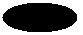 | 3 | 10467 |
| SAGE Panc1 | 80 | 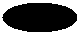 | 2 | 24879 |
| SAGE HX | 279 | 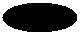 | 9 | 32157 |
| SAGE H126 | 215 | 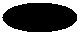 | 7 | 32420 |
| SAGE Duke H54 lacZ | 14 | 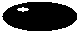 | 1 | 67101 |
| SAGE Duke H54 EGFRvIII | 87 | 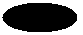 | 5 | 57164 |
| SAGE Duke H392 | 17 | 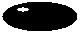 | 1 | 57529 |
| SAGE Duke GBM H1110 | 42 | 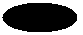 | 3 | 70061 |
| SAGE SW837 | 16 | 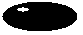 | 1 | 60986 |
| SAGE RKO | 57 | 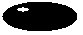 | 3 | 52064 |
| SAGE PR317 prostate tumor | 15 | 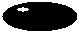 | 1 | 65109 |
| SAGE pooled GBM | 80 | 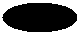 | 5 | 61841 |
| SAGE BB542 whitematter | 84 | 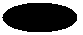 | 8 | 94806 |
| SAGE NHA(5th) | 95 | 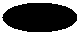 | 5 | 52196 |
| SAGE normal pool(6th) | 31 | 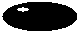 | 2 | 63064 |
| SAGE Panc 91-16113 | 88 | 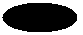 | 3 | 33941 |
| SAGE Panc 96-6252 | 27 | 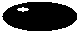 | 1 | 35745 |
| SAGE CV10363-3 | 25 | 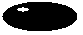 | 1 | 38938 |
| SAGE T098 | 20 | 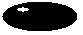 | 1 | 49005 |
| SAGE SciencePark MCF7 Control 0h | 16 | 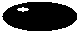 | 1 | 61079 |
| SAGE Ped GBM1062 | 33 | 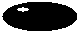 | 2 | 59935 |
| SAGE HOSE 4 | 82 | 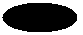 | 4 | 48413 |
| SAGE Duke HMVEC | 152 | 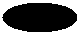 | 8 | 52532 |
| SAGE Duke HMVEC + VEGF | 155 | 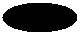 | 9 | 57926 |
| SAGE mammary epithelium | 81 | 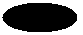 | 4 | 49167 |
| SAGE OVT-8 | 29 | 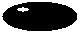 | 1 | 33575 |
| SAGE Duke 40N | 140 | 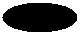 | 1 | 7142 |
| SAGE Duke 48N | 248 | 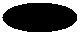 | 3 | 12091 |
| SAGE Duke H247 Hypoxia | 125 | 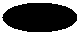 | 9 | 71937 |
| SAGE DCIS 2 | 173 | 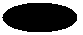 | 5 | 28888 |
| SAGE Br N | 106 | 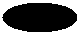 | 4 | 37558 |
| SAGE IOSE29-11 | 61 | 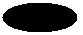 | 3 | 48498 |
| SAGE Duke H1043 | 26 | 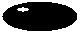 | 2 | 76673 |

### Example 6

### Comparing expression of ARP with Vascular Endothelial Growth Factor (VEGF) Expression

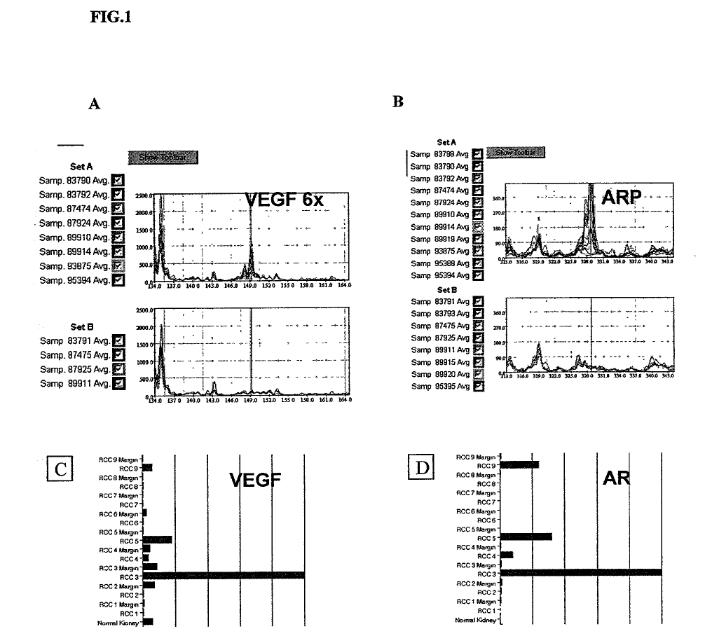

Paradis and coworkers assessed VEGF expression in a large series of renal tumors with a long follow-up, correlated with the usual histo-prognostic factors and survival. Their study revealed that in the group of clear cell RCCs, VEGF expression was positively correlated with both nuclear grade (P=0.05) and size of the tumor (P=0.05). Furthermore, a significant correlation was observed between VEGF expression and microvascular count (P=0.04). Finally, cumulative survival rate was significantly lower in the group of patients with clear cell RCCs expressing VEGF (log rank test, P=0.01). In the Cox model, VEGF expression was a significant independent predictor of outcome, as well as stage and nuclear grade. (Paradis V, Lagha N B, Zeimoura L, Blanchet P, Eschwege P, Ba N, Benoit G, Jardin A, Bedossa P. Expression of vascular endothelial growth factor in renal cell carcinomas. Virchows Arch April 2000;436(4):351-6). The expression profile of VEGF was compared with the expression profile of ARP. As shown in FIG. 3, ARP overexpression is higher and more specific than VEGF, indicating that it could be used as a better clinical marker and that more efficacious and specific therapeutics can be directed at regulating ARP expression. These results also indicate that a treatment that modulates the expression of VEGF and ARP at the same time may achieve synergistic effects. An example of a treatment that can mitigate the effects of the expression of both VEGF and ARP is a bispecific antibody directed both these targets. The bi-specific antibody contemplated to be within the scope of claims for this invention may be an antibody generated by quadroma technology, or by chemical cross-linking of mono-specific antibodies (one directed against VEGF, the other against ARP) or a bi-specific single chain antibody dimer. Formulations of single chain antibodies may include, but not limited to: VL(a)-Linker-VH(a)-Linker-VL(b)-Linker-VH(b). For examples of bispecific antibodies see: U.S. Pat. No. 6,030,792 by Ottemess et al., the references therein included here, Multivalent single chain antibodies, U.S. Pat. Nos. 5,892,020, 5,877,291 by Mezes et al., U.S. Pat. No. 6,071,515: Dimer and multimer forms of single chain polypeptides by Mezes et al., and U.S. Pat. No. 6,121,424: Multivalent antigen-binding proteins by Whitlow et al. See FIG. 1.

TABLE 10
Genecalling results from Job 36320 - all kidney cancer vs all Kidney NAT

| | ARP - Human | | | | | | | | | | | | | |
|---|---|---|---|---|---|---|---|---|---|---|---|---|---|---|
| | angiopoietin-related | | | | | | | | | | | | | |
| | protein. (growth factor) | | | | 67.1 | 1 | 1 of 2 | | | | | | | |
| gbh af153606 | | Band | Fold | Set | Set | Set | Visual | | | Trap Info | | | | |
| Band ID | Offset | Confirm | Diff. | Sig | A | B | Inspection | 3 of 9 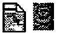 | | Score | J1 | J2 | R1 | R2 |
| d0p0-69 5 | 493 | unconf | 2.3 | .91 | 108.4 (33.6) | 47.3 (6.1) | 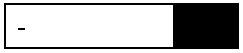 | 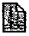 | | | | | | |
| p0c0-131 2(131.2) | 393 | Pass- Complete | 67.3 | .96 | 853.2 (444) | 12.7 (2.6) | 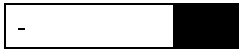 | 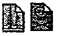 | 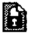 | 1 comment | | | | |
| p0c0-131 1 | 896 | unconf | 5.2 | 1 | 398.9 (143.6) | 76.2 (9.7) | 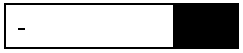 | 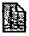 | | | | | | |

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to, be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112;
(b) a variant of a mature form of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112; and
(d) a variant of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112;
(b) a variant of a mature form of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112;
(d) a variant of an amino acid sequence selected from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111.

9. The nucleic acid molecule of claim 5, wherein said nucleic acid molecule comprises a nucleotide sequence selected from the group consisting of
(a) a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111;
(b) a nucleotide sequence differing by one or more nucleotides from a nucleotide sequence selected from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 and 111, or a complement of said nucleotide sequence.

11. The nucleic acid molecule of claim 5, wherein the nucleic acid molecule comprises a nucleotide sequence selected from the group consisting of
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that immunospecifically-binds to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule, thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(b) contacting the cell with said agent; and
(c) determining whether the agent modulates expression or activity of said polypeptide, whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

24. The method of claim 23, wherein said subject is a human.

25. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

26. The method of claim 25, wherein said subject is a human.

27. A method of treating or preventing a NOVX-associated disorder, said method comprising administering to a subject in which such treatment or prevention is desired the antibody of claim 15 in an amount sufficient to treat or prevent said NOVX-associated disorder in said subject.

28. The method of claim 27, wherein the subject is a human.

29. A pharmaceutical composition comprising the polypeptide of claim 1 and a pharmaceutically-acceptable carrier.

30. A pharmaceutical composition comprising the nucleic acid molecule of claim 5 and a pharmaceutically-acceptable carrier.

31. A pharmaceutical composition comprising the antibody of claim 15 and a pharmaceutically-acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a NOVX-associated disorder, wherein said therapeutic is selected from the group consisting of a NOVX polypeptide, a NOVX nucleic acid, and a NOVX antibody.

36. A method for screening for a modulator of activity or of latency or predisposition to a NOVX-associated disorder, said method comprising:
(a) administering a test compound to a test animal at increased risk for a NOVX-associated disorder, wherein said test animal recombinantly expresses the polypeptide of claim 1;
(b) measuring the activity of said polypeptide in said test animal after administering the compound of step (a);
(c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a NOVX-associated disorder.

37. The method of claim 36, wherein said test animal is a recombinant test animal that expresses a test protein transgene or expresses said transgene under the control of a promoter at an increased level relative to a wild-type test animal, and wherein said promoter is not the native gene promoter of said transgene.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

39. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

40. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, and 112, or a biologically active fragment thereof.

41. A method of treating a pathological state in a mammal, the method comprising administering to the mammal the antibody of claim 15 in an amount sufficient to alleviate the pathological state.

42. A method of a treating a disorder in a subject, said method comprising administering to a subject in need thereof a therapeutically effective amount of a compound which decreases IL-8 expression or activity in said subject, thereby treating said disorder in said subject.

43. The method of claim 42, wherein said disorder is an inflammatory disorder.

44. The method of claim 42, wherein said disorder is cancer.

45. The method of claim 42, wherein said disorder is a demyelination disease.

46. The method of claim 42, wherein the compound is a IL-8 antibody, a IL-8 antisense nucleic, or a nucleic acid that decreases expression of a nucleic acid that encodes a IL-8 polypeptide.

47. The method of claim 42, wherein the subject is a rodent or human.

48. The method of claim 42, wherein the compound is administered to the subject in association with a transfection agent.

49. The method of claim 42, wherein the administering is by a route selected from the group consisting of intraperitoneal, subcutaneous, nasal, intravenous, oral and transdermal delivery.

50. The method of claim 42, wherein the administering is intravenous.

51. A method of identifying a ligand for the peroxisome proliferator-activated receptor gamma (PPAR γ) receptor, the method comprising;
(a) providing a test cell population comprising a cell capable of expressing angiopoietin related protein (ARP)
(b) contacting the test cell population with a test agent;
(c) measuring expression of ARP in the test cell population;
(d) comparing the expression of ARP test cell population to the expression of ARP in a reference cell population which has not been exposed to the test agent; and
(e) identifying a difference in expression levels of the ARP, if present, in the test cell population and reference cell population,
wherein a increase in ARP expression in the test cell population as compared to the reference cell population indicates that the test agent is a ligand for the PPAR γ receptor.

52. The method of claim 51, wherein the test cell population is provided in vitro.

53. The method of claim 51, wherein the test cell population is provided ex vivo from a mammalian subject.

54. The method of claim 51, wherein the test cell is provided in vivo in a mammalian subject.

55. The method of claim 51, wherein the test cell population is derived from a human or rodent subject.

56. The method of claim 51, wherein the test cell includes a adipocyte.

57. A PPAR γreceptor ligand identified according to the method of claim 51.

58. A pharmaceutical composition comprising the PPAR γ receptor ligand of claim 57.

59. A method of identifying a therapeutic agent, the method comprising;
(a) providing a test cell population comprising a cell capable of expressing ARP
(b) contacting the test cell population with a test agent;
(c) measuring expression of ARP in the test cell population;
(d) comparing the expression of the ARP in the test cell population to the expression of ARP in a reference cell population comprising at least one cell whose disease status to is known; and
(e) identifying a difference in expression levels of ARP, if present, in the test cell population and reference cell population,
thereby identifying a therapeutic agent.

60. The method of claim 59, wherein the test cell population is provided in vitro.

61. The method of claim 59, wherein the test cell population is provided ex vivo from a mammalian subject.

62. The method of claim 59, wherein the test cell population is provided in vivo in a mammalian subject.

63. The method of claim 59, wherein the test cell population is derived from a human or rodent subject.

64. The method of claim 59, wherein the test cell population includes a kidney cell.

65. The method of claim 59, wherein the expression of the nucleic acid sequences in the test cell population is decreased as compared to the reference cell population.

66. The method of claim 59, wherein the expression of the nucleic acid sequences in the test cell population is increased as compared to the reference cell population.

67. A method of diagnosing or determining the susceptibility to clear cell renal carcinoma in a subject, the method comprising:
(a) providing from the subject a test cell population comprising cells capable of expressing of ARP;
(b) measuring expression of ARP in the test cell population; and
(c) comparing the expression of ARP in the test cell population to the expression of ARP in a reference cell population comprising at least one cell from a subject not suffering from clear cell renal carcinoma; and
(d) identifying a difference in expression levels of ARP, if present, in the test cell population and reference cell population,
wherein an increase of expression of ARP in the test cell population compared to the reference cell population indicated that the subject is suffering from or susceptible to clear cell renal carcinoma.

68. A method of treating a renal disorder in a subject, the method comprising administering to the subject in need thereof an agent that decreases the expression or the activity ARP

69. The method of claim 68, wherein the renal disorder is kidney cancer, polycystic kidney disease, renal dysplasia, or kidney degenerative disease.

70. The method of claim 69, wherein the kidney cancer is renal cell carcinoma or wilms tumor.

71. The method of claim 69, wherein the kidney degenerative disease is chronic kidney failure.

72. A method of assessing the efficacy of a treatment of a kidney disorder in a subject, the method comprising:
(a) providing from the subject a test cell population comprising cells capable of expressing ARP;
(b) detecting expression ARP in the test cell population;
(c) comparing the expression ARP in the test cell population to the expression of ARP in a reference cell population comprising at least one cell from a subject not suffering from the kidney disorder; and
(e) identifying a difference in expression levels of ARP, if present, in the test cell population and reference cell population,
wherein a similarity in ARP expression in the test cell population and the reference population indicate the treatment is efficacious.

73. A method of diagnosing or determining the susceptibility a inflammatory disorder in a subject, the method comprising:
(a) providing from the subject a test cell population comprising cells capable of expressing of ARP;
(b) measuring expression of ARP in the test cell population; and
(c) comparing the expression of ARP in the test cell population to the expression of ARP in a reference cell population comprising at least one cell from a subject not suffering from the inflammatory disorder; and
(d) identifying a difference in expression levels of ARP, if present, in the test cell population and reference cell population,
wherein an increase of expression of ARP in the test cell population compared to the reference cell population indicated that the subject is suffering from or susceptible to the inflammatory disorder.

74. A method of treating a inflammatory disorder in a subject, the method comprising administering to the subject in need thereof an agent that decreases the expression or the activity ARP

75. The method of claim 74, wherein the inflammatory disorder is a disorder of the pulmonary system

76. The method of claim 74, wherein the inflammatory disorder is asthma, allergy, emphysema, arthritis or Chronic Obstructive Pulmonary Disease.

77. A method of assessing the efficacy of a treatment of a inflammatory disorder in a subject, the method comprising:
(a) providing from the subject a test cell population comprising cells capable of expressing ARP;
(b) detecting expression ARP in the test cell population;
(c) comparing the expression ARP in the test cell population to the expression of ARP in a reference cell population comprising at least one cell from a subject not suffering from the inflammatory disorder; and
(e) identifying a difference in expression levels of ARP, if present, in the test cell population and reference cell population,
wherein a similarity in ARP expression in the test cell population and the reference population indicate the treatment is efficacious.
